SEQUENCE LISTING (4 sequences)

SEQ ID NO: 1 - DNA, 2994 bp, Streptococcus pneumoniae
atgattgtaggagcagtggtatttggaacgtctcctgttttagctcaagaaggggcaagtgagcaacctctggcaaatgaaactcaactttcgggggagagctcaaccctaactgatacagaaaagagccagccttcttcagagactgaactttctggcaataagcaagaacaagaaaggaaagataagcaagaagaaaaaattccaagagattactatgcacgagatttggaaaatgtcgaaacagtgatagaaaaagaagatgttgaaaccaatgcttcaaatggtcagagagttgatttatcaagtgaactagataaactaaagaaacttgaaaacgcaacagttcacatggagtttaagccagatgccaaggccccagcattctataatctcttttctgtgtcaagtgctactaaaaaagatgagtacttcactatggcagtttacaataatactgctactctagaggggcgtggttcggatgggaaacagttttacaataattacaacgatgcacccttaaaagttaaaccaggtcagtggaattctgtgactttcacagttgaaaaaccgacagcagaactacctaaaggccgagtgcgcctctacgtaaacggggtattatctcgaacaagtctgagatctggcaatttcattaaagatatgccagatgtaacgcatgtgcaaatcggagcaaccaagcgtgccaacaatacggtttgggggtcaaatctacagattcggaatctcactgtgtataatcgtgctttaacaccagaagaggtacaaaaacgtagtcaactttttaaacgctcagatttagaaaaaaaactacctgaaggagcggctttaacagagaaaacggacatattcgaaagcgggcgtaacggtaaaccaaataaagatggaatcaagagttatcgtattccagcacttctcaagacagataaaggaactttgatcgcaggtgcagatgaacgccgtctccattcgagtgactggggtgatatcggtatggtcatcagacgtagtgaagataatggtaaaacttggggtgaccgagtaaccattaccaacttacgtgacaatccaaaagcttctgacccatcgatcggttcaccagtgaatatcgatatggtgttggttcaagatcctgaaaccaaacgaatcttttctatctatgacatgttcccagaagggaagggaatctttggaatgtcttcacaaaaagaagaagcctacaaaaaaatcgatggaaaaacctatcaaatcctctatcgtgaaggagaaaagggagcttataccattcgagaaaatggtactgtctatacaccagatggtaaggcgacagactatcgcgttgttgtagatcctgttaaaccagcctatagcgacaagggggatctatacaagggtaaccaattactaggcaatatctacttcacaacaaacaaaacttctccatttagaattgccaaggatagctatctatggatgtcctacagtgatgacgacgggaagacatggtcagcgcctcaagatattactccgatggtcaaagccgattggatgaaattcttgggtgtaggtcctggaacaggaattgtacttcggaatgggcctcacaagggacggattttgataccggtttatacgactaataatgtatctcacttaaatggctcgcaatcttctcgtatcatctattcagatgatcatggaaaaacttggcatgctggagaagcggtcaacgataaccgtcaggtagacggtcaaaagatccactcttctacgatgaacaatagacgtgcgcaaaatacagaatcaacggtggtacaactaaacaatggagatgttaaactctttatgcgtggtttgactggagatcttcaggttgctacaagtaaagacggaggagtgacttgggagaaggatatcaaacgttatccacaggttaaagatgtctatgttcaaatgtctgctatccatacgatgcacgaaggaaaagaatacatcatcctcagtaatgcaggtggaccgaaacgtgaaaatgggatggtccacttggcacgtgtcgaagaaaatggtgagttgacttggctcaaacacaatccaattcaaaaaggagagtttgcctataattcgctccaagaattaggaaatggggagtatggcatcttgtatgaacatactgaaaaaggacaaaatgcctataccctatcatttagaaaatttaattgggactttttgagcaaagatctgatttctcctaccgaagcgaaagtgaagcgaactagagagatgggcaaaggagttattggcttggagttcgactcagaagtattggtcaacaaggctccaacccttcaattggcaaatggtaaaacagcacgcttcatgacccagtatgatacaaaaaccctcctatttacagtggattcagaggatatgggtcaaaaagttacaggtttggcagaaggtgcaattgaaagtatgcataatttaccagtctctgtggcgggcactaagctttcgaatggaatgaacggaagtgaagctgctgttcatgaagtgccagaatacacaggcccattagggacatccggcgaagagccagctccaacagtcgagaagccagaatacacaggcccactagggacatccggcgaagagccagccccgacagtcgagaagccagaatacacaggcccactagggacagctggtgaagaagcagctccaacagtcgagaagccagaatttacagggggagttaatggtacagagccagctgttcatgaaatcgcagagtataagggatctgattcgcttgtaactcttactacaaaagaagattatacttacaaagctcctcttgctcagcaggcacttcctgaaacaggaaacaaggagagtgacctcctagcttcactaggactaacagctttcttccttggtctgtttacgctagggaaaaagagagaacaataa

SEQ ID NO: 2 - protein, 997 aa, Streptococcus pneumoniae
MIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 3 - protein, 470 aa, Streptococcus pneumoniae
ALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSK

SEQ ID NO: 4 - protein, 470 aa, Streptococcus pneumoniae; identical to SEQ ID NO: 3

(19) United States
(12) Patent Application Publication
(10) Pub. No.: US 2011/0280813 A1
(43) Pub. Date: Nov. 17, 2011

(54) NEURAMINIDASE INHIBITORS AND USES THEREOF

(76) Inventors: Alice Prince, Larchmont, NY (US); Liang Tong, Scarsdale, NY (US)

(21) Appl. No.: 13/128,688

(22) PCT Filed: Nov. 13, 2009
(86) PCT No.: PCT/US09/64393

§ 371 Date: Aug. 1, 2011

Related U.S. Application Data
(60) Provisional application No. 61/114,104, filed on Nov. 13, 2008.

(51) Int. Cl. A61K 8/64; A61K 31/7105; C40B 30/02; C07K 2/00; C07K 16/40; A61K 31/4015; A61K 38/08; A01N 43/16; A61K 31/351; A01N 47/44; A61K 31/196; A01N 37/30; A61K 31/215; A61K 31/713; A61K 31/711; A01N 57/16; A61K 39/395; A01N 37/18; A61P 31/04; A01P 1/00; A61K 31/545; A61K 31/43; A61K 31/65; A61K 8/49; A61K 8/60; A61K 8/43; A61K 8/42; A61P 11/00; A61Q 11/00; A01N 43/36

(52) U.S. Cl. 424/49; 514/425; 514/44 A; 506/8; 530/300; 530/387.9; 514/2.4; 514/459; 514/573; 514/529; 424/158.1; 514/2.8; 514/2.6; 514/200; 514/192; 514/152; 514/1.8

(57) ABSTRACT

The invention is related to various methods for inhibiting or reducing biofilm formation, treating a biofilm production-related disorder, preventing biofilm formation, and screening for neuraminidase inhibitors.

### GOVERNMENT INTERESTS

The work described herein was supported in whole, or in part, by National Institute of Health Grant No. RO1 DK29693. Thus, the United States Government has certain rights to the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Many respiratory pathogens including Hemophilus influenzae (H. influenzae), Streptococcus pneumoniae (S. pneumoniae), and Pseudomonas aeruginosa (P. aeruginosa) express neuraminidases that can cleave α-2,3 linked sialic acids from glycoconjugates. As mucosal surfaces are heavily sialylated, neuraminidases have been thought to modify epithelial cells by exposing potential bacterial receptors. However, in contrast to neuraminidase produced by the influenza virus, a role for bacterial neuraminidase in pathogenesis has not been clearly established, especially as it pertains to regulating the formation of biofilms.

### SUMMARY OF THE INVENTION

One aspect of the present invention provides a method for reducing or inhibiting bacterial biofilm formation where a surface is contacted with a bacterial neuraminidase inhibitor for a sufficient time so as to bacterial modulate neuraminidase activity. The neuraminidase inhibitor modulates the activity or the expression of a neuraminidase, thereby resulting in inhibiting or reducing the formation of the biofilm. In one embodiment, the surface comprises a biofilm. A biofilm can be produced by a bacterium, a virus, a protozoan, a fungus, or by any combination of the organisms mentioned. In one embodiment, the biofilm is a bacterial biofilm. In some embodiments, the neuraminidase is a bacterial neuraminidase. In other embodiments the neuraminidase inhibitor targets bacterial neuraminidases. In some embodiments of the invention, the expression or the activity of the neuraminidase in the biofilm is reduced after the neuraminidase inhibitor is applied to a surface. In one embodiment, the neuraminidase inhibitor is an antibody that specifically binds to the NanA protein of S. pneumoniae or a fragment thereof; an antisense RNA or antisense DNA that inhibits expression of a NanA polypeptide; a siRNA that specifically targets a NanA gene, a peptide comprising at least 10 amino acids of SEQ ID NO: 2 wherein the peptide competes with endogenous NanA for ligand binding; or a combination thereof. In specific embodiments, the neuraminidase inhibitor comprises oseltamivir, peramivir, zanamivir, or a variant thereof.

In further embodiments, he neuraminidase inhibitor is a compound comprising Formula (I):

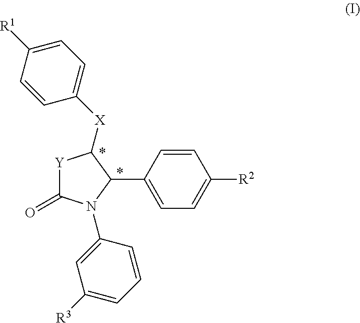

wherein,

R¹ is H, halogen, cyano, azido, nitro, C₁-C₆ alkyl, or C₁-C₆ alkoxy;

R² is H, halogen, cyano, azido, nitro, C₁-C₆ alkyl, or C₁-C₆ alkoxy;

R³ is H, —CO₂R⁴ or —CON(R⁴)₂;

each R⁴ is, independently, H or C₁-C₆ alkyl;

X is —CH₂—, —(C═O)—, —(C═NH)—, —(C═N—O—C₁-C₆-alkyl)-, or —(C═S)—; and

Y is —(C═O)—, —(C═NH)—, —(C═N—O—C₁-C₆-alkyl)-, or —(C═S)—,

or a pharmaceutically acceptable salt or hydrate thereof.

In some embodiments, the neuraminidase inhibitor is a compound comprising Formula (A):

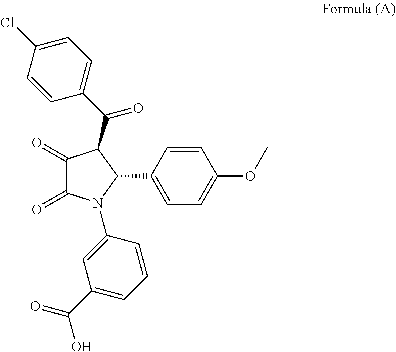

In some embodiments, the neuraminidase inhibitor is a compound comprising Formula (X):

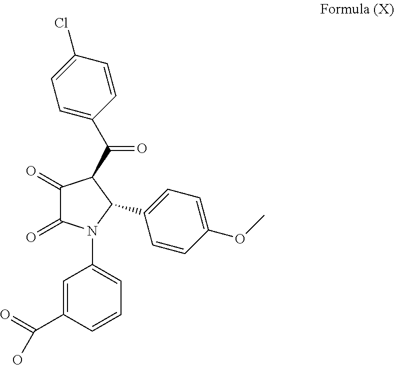

Any biofilm-forming organism can comprise the biofilm mass. In certain embodiments, those organisms are viruses, bacteria, protozoa, and fungi. In various embodiments, the biofilm comprises a Gram-negative bacterium. In other embodiments, the biofilm comprises a Gram-positive bacterium. In some embodiments, the bacterium is Streptococcus and in other embodiments the Gram-positive bacteria are Streptococcus (e.g., S. pneumoniae) while in other embodiments, the Gram-negative bacteria are Haemophilus (e.g., Haemophilus influenzae); Pseudomonas (P. aeruginosa), or Vibrio (e.g., Vibrio cholerae). A biofilm can be found on various surfaces and such a surface can be contacted with a neuraminidase inhibitor. In one embodiment, the surface comprises a cellular surface of a subject, an in vitro surface, or an oral surface of a subject. In some useful embodiments, the surface comprises a prosthetic graft, a catheter, a wound dressing, a wound site, a medical device, a contact lens, an implanted device, an oral device, a pipe, or industrial equipment. In further embodiments of the invention, the contacting comprises administering the neuraminidase inhibitor to a subject via subcutaneous, intra-muscular, intra-peritoneal, or intravenous injection; infusion; oral, nasal, or topical delivery; or a combination thereof. In some embodiments, the subject is a human, mouse, rat, bird, dog, cat, cow, horse, or pig. In another embodiment, the neuraminidase inhibitor is applied to the surface of a prosthetic graft to be introduced into a subject. In other embodiments, the neuraminidase inhibitor is applied to the surface of a catheter to be implanted into a subject. In yet further embodiments, the neuraminidase inhibitor is applied to the surface of a wound dressing to be applied on or in a subject. In other embodiments, the neuraminidase inhibitor is applied to the surface of a wound site on a subject. In additional embodiments, the neuraminidase inhibitor is applied to the surface of a medical device to be implanted or inserted into a subject. The subject in many of these instances can harbor the biofilm or has the propensity to form a biofilm. The neuraminidase inhibitor also can be administered to the subject prior to, or during, or after the implantation or insertion of a prosthetic graft, medical device, or a catheter, the application of the wound dressing or to the wound site.

The neuraminidase inhibitor according to the method of the invention can be applied to a surface where a biofilm has formed. In one embodiment, the surface comprises a contact lens, an implanted device, an oral device, a pipe, or industrial equipment. In other embodiments, industrial equipment is found in a GMP facility. In some embodiments, the industrial equipment comprises a plumbing system. In other embodiments, the surface where a biofilm has formed comprises an oral surface of a subject. In some embodiments, the biofilm is associated with dental caries while in other embodiments it is associated with periodontal disease. In some embodiments, the neuraminidase inhibitor is in a formulation of a paste, a liquid, a powder, a gel, or a tablet. According to an embodiment of the invention, the neuraminidase inhibitor can be in a paste formulation that can further comprise an abrasive, such as toothpaste. In other embodiments, the neuraminidase inhibitor can be a liquid formulation, such as a mouthwash.

A second therapeutic composition, different than the neuraminidase inhibitor, can also be administered to a subject. In some embodiments of the invention, administration occurs sequentially while in others administration occurs simultaneously. In various embodiments, the therapeutic composition comprises an antibiotic. In yet additional embodiments, the antibiotic comprises a cephalosporin, a macrolide, a penicillin, a quinolone, a sulfonamide, and a tetracycline, or any combination of the listed antibiotics.

Another aspect of the current invention provides for methods of treating a biofilm production-related disorder in a subject in need thereof. The method comprises administering to the subject an effective amount of a bacterial neuraminidase inhibitor that reduces biofilm formation in the subject. In one embodiment, the neuraminidase inhibitor is a compound comprising Formula (X), Formula (I), or Formula (A), as described herein. In another embodiment, the neuraminidase inhibitor is an antibody that specifically binds to the NanA protein of S. pneumoniae or a fragment thereof; an antisense RNA or antisense DNA that inhibits expression of a NanA polypeptide; a siRNA that specifically targets a NanA gene, a peptide comprising at least 10 amino acids of SEQ ID NO: 2 wherein the peptide competes with endogenous NanA for ligand binding; or a combination thereof. A reduction or inhibition in the growth of biofilm production-related bacteria in the subject can then be determined. A reduction in bacterial growth is indicative of the reduction in or inhibition of biofilm production in the subject. Thus, the method is useful for treating the biofilm production-related disorder. In one embodiment, the subject being treated is a mammal, whereas in other embodiments the subject is a human. In some embodiments, the subject can also be a mouse, rat, bird, dog, cat, cow, horse, or pig. A biofilm production-related disorder of the invention can be a disorder or disease that is characterized by a disease-related growth of bacteria, which can result in the establishment of a biofilm. In other embodiments, the disorder affects an epithelial surface, a mucosal surface, or a combination of those surfaces. In further embodiments of the invention, the surface is a lung surface. In some embodiments, the biofilm production-related disorder is caused by a bacterium, such as a Gram-negative or Gram-positive bacterium. In other embodiments, the bacterium comprises Streptococcus (such as S. pneumoniae); Haemophilus (such as Haemophilus influenzae); or Vibrio (such as Vibrio cholerae). In some embodiments, the bacterium is S. pneumoniae. In yet further embodiments, the disorder is pneumonia, cystic fibrosis (CF), otitis media, or chronic obstructive pulmonary disease (COPD). According to the invention, in additional embodiments, the disorder is a medical device-related bacterial infection. The infection arises from the device being implanted or inserted into the subject.

The reduction in bacterial growth can be indicative of the reduction in or inhibition of biofilm production in a subject. In some embodiments, the growth of biofilm production-related bacteria can be determined by measuring the biofilm production-related bacteria in a biological sample. In other embodiments, the presence or growth of biofilm production-related bacteria is measured by detecting the presence of antigens of biofilm production-related bacteria in a biological sample. The biological sample can be blood, serum, sputum, lacrimal secretions, semen, urine, vaginal secretions, or a tissue sample. For example, an antibody to S. pneumoniae components can be used as a test for colonization/infection in a subject afflicted with a biofilm production-related condition or disorder, wherein the presence of Streptococcus antigens is detected in a biological sample, such as blood. These antibodies can be generated according to methods well established in the art or can be obtained commercially (for example, from Abcam, Cambridge, Mass.; Cell Sciences Canton, M A; Novus Biologicals, Littleton, Colo.; or GeneTex, San Antonio, Tex.). The reduction in the growth of biofilm production-related bacteria can also be measured by chest x-rays, or by a pulmonary function test (PFT), such as spirometry or forced expiratory volume (FEV₁) as described below.

In various embodiments of the invention, the biofilm comprises viruses, protozoa, fungi, or bacteria, such as a Gram-positive bacterium and a Gram-negative bacterium. In some embodiments, the bacterium is Streptococcus (such as S. pneumoniae); Haemophilus (such as Haemophilus influenzae); or Vibrio (such as Vibrio cholerae). In other embodiments, the bacterium is S. pneumoniae. According to the invention, a neuraminidase inhibitor that is applied to a surface likely to develop a biofilm modulates the activity or expression of a targeted neuraminidase, such as a bacterial neuraminidase. In some embodiments, the expression of the neuraminidase is reduced, while in other embodiments, the activity of the neuraminidase is reduced. In various embodiments, the neuraminidase inhibitor is applied as a formulation comprising a paste, liquid, powder, gel, or tablet. In certain embodiments, the industrial surface to which the neuraminidase inhibitor is applied is part of a plumbing system.

A useful neuraminidase inhibitor according to the invention can be any compound, small molecule, peptide, protein, aptamer, ribozyme, RNAi, or antisense oligonucleotide, and the like. In one embodiment, the neuraminidase inhibitor is a viral neuraminidase inhibitor. In other embodiments, the viral neuraminidase inhibitor comprises oseltamivir, peramivir, zanamivir, or a variant thereof.

Other aspects of the invention provide screening methods for identifying a compound that modulates neuraminidase activity. The method comprises providing an electronic library of test compounds stored on a computer, then providing atomic coordinates for at least twenty amino acid residues of Streptococcus neuraminidase listed in Table 2, wherein the coordinates have a root mean square deviation therefrom, with respect to at least 50% of the Cα atoms, of not greater than about 2 Å, in a computer readable format. The atomic coordinates are then converted into electrical signals readable by a computer processor to generate a three-dimensional model of the neuraminidase. A data processing method is then performed, wherein electronic test compounds from the library are superimposed upon the three-dimensional model of the neuraminidase. Whether a test compound fits into the binding pocket of the three-dimensional model of the neuraminidase is subsequently determined, enabling the identification of which compound would modulate the activity of the neuraminidase.

In another aspect of the invention, the method for identifying a compound that modulates neuraminidase activity comprises providing an electronic library of test compounds stored on a computer, then providing atomic coordinates listed in Table 2 in a computer readable format for at least 5, 6, 7, 8, 9, 10, 11, or 12 amino acid residues located within about 10 Å of the Streptococcus neuraminidase active site, wherein the residues comprise 5 or more of the following residues: Arg347, Arg366, Asp372, Asp417, Ile442, Phe443, Phe565, Tyr590, Gln602, Glu647, Arg663, Tyr695, Tyr752, or Arg 721. The atomic coordinates are then converted into electrical signals readable by a computer processor to generate a three-dimensional model of the neuraminidase active site. A data processing method is then performed, wherein electronic test compounds from the library are superimposed upon the three-dimensional model of the neuraminidase active site. Whether a test compound fits into the binding pocket of the three-dimensional model of the neuraminidase is subsequently determined, enabling the identification of which compound would modulate the activity of the neuraminidase.

The methods described above can further comprise obtaining or synthesizing the compound determined to to bind to NanA or modulate the neuraminidase activity; contacting a bacterium with the compound in vitro; and determining whether the compound modulates neuraminidase activity using a biological assay. In one embodiment, the bacterium is a Gram-negative bacterium. In a further embodiment, the bacterium is a Gram-positive bacterium. In another embodiment, the bacterium is Streptococcus (i.e., S. pneumoniae), Pseudomonas (such as P. aeruginosa), Haemophilus, (i.e Haemophilus influenzae), or Vibrio (such as Vibrio cholerae). In further embodiments, the biological assay comprises a biofilm assay, an adherence assay, or a combination of the two mentioned assays. In one embodiment, the biological assay entails contacting a surface harboring a biofilm (for example, produced by a pathogenic organism, such as a bacterium) in vitro with a test neuraminidase inhibitor, and then determining whether the test neuraminidase inhibitor inhibits biofilm formation at the surface. Inhibition of biofilm formation is indicative of the ability of the test neuraminidase inhibitor to inhibit the pathogenic infection, such as a bacterial infection. In one embodiment, the pathogen is a Gram-positive bacterium, such as S. pneumoniae. Thus, the method can be used for identifying neuraminidase inhibitors that can inhibit a pathogenic infection.

In a further aspect, the invention provides a compound identified by the screening methods above, wherein the compound binds to the neuraminidase active site, and comes within 10 Å of amino acid residues listed in Table 3. In one embodiment, the compound inhibits or reduces biofilm formation. In another embodiment, the compound is a peptide that binds to a neuraminidase, such as an anti-neuraminidase antibody or a binding fragment thereof. In a further embodiment, the peptide interacts with a protein having the amino acid sequence of SEQ ID NO: 2. In some embodiments, the compound interacts with a protein having the amino acid sequence of SEQ ID NO: 2.

According to the methods of the present invention, a candidate or test neuraminidase inhibitor can be any compound, small molecule, peptide, protein, aptamer, ribozyme, RNAi, or antisense oligonucleotide, and the like. In one embodiment, the test inhibitor is a peptide that binds to a neuraminidase. In further embodiments, the neuraminidase can be a bacterial neuraminidase. In other embodiments, the test inhibitor is an anti-neuraminidase antibody or a binding fragment thereof. In specific embodiments of the invention, the test inhibitor is a peptide that interacts with a protein comprising the amino acid sequence of SEQ ID NO: 2. In various embodiments, the test inhibitor is a viral neuraminidase inhibitor while in other embodiments the viral neuraminidase inhibitor comprises oseltamivir, peramivir, zanamivir, or a variant thereof. In some embodiments, the neuraminidase inhibitor is compound of Formula (X), Formula (A), or Formula (I), as described herein. In further embodiments of the invention, the test inhibitor is a peptide that interacts with a protein having the amino acid sequence of SEQ ID NO: 2.

### BRIEF DESCRIPTION OF THE FIGURES

FIG. 1 shows schematic representations of the structure of S. pneumoniae NanA. FIG. 1A depicts the β-strands shown in cyan, α-helices in yellow, and connecting loops in magenta. The inhibitor NANA is shown as a stick model, in black for carbon atoms. FIG. 1B depicts the final 2Fₒ-F_{c} electron density at 1.7 Å resolution for the inhibitor NANA, contoured at 1σ, in a boat conformation. FIG. 1C is a stereo drawing showing detailed interactions between NANA (black) or DANA (orange) with the active site of NanA. FIG. 1D show the molecular surface of NanA in the active region, colored by electrostatic potential with NANA. The figures were created with Pymol [A76] and Grasp [A77].

FIG. 2 shows schematic representations of the structure of P. aeruginosa NanPs. In FIG. 2A, the β-strands are shown in green, α-helices in yellow, and connecting loops in magenta. FIG. 2B depicts the molecular surface of NanPs in the active site region, colored by electrostatic potential. The view is the same as that of FIG. 1D, and the position of NANA bound to NanA is shown for reference. FIG. 2C shows the structural differences between the active site regions NanPs (in green) and NanA (in cyan). Residue numbers in green are for NanPs, and those in blue for NanA.

FIG. 3 is a bar graph showing the activity of P. aeruginosa neuraminidase mutations. Site-directed mutations were made within the active site of P. aeruginosa neuraminidase or truncation in the C-terminus (deleting residues 334-438) and purified protein used to determine neuraminidase activity compared to wild-type enzyme (WT, control) using the fluorogenic substrate 2′-(4-methylumbelliferyl)-α-D-N-acetylneuraminic acid. *p-value<0.05.

FIG. 4 is a schematic representation showing the phylogenetic analysis of neuraminidases. Shown is the unrooted phylogenetic network for our broad survey of the neuraminidase phylogeny. It is the strict consensus of 3 most parsimonious trees (step s=45885, consistency index=0.326, retention index=0.385, rescaled consistency=0.125). Solid circles indicate branches with bootstrap values greater than 80%. Grey circles indicate bootstrap values between 50-80%. Open circles indicate bootstrap values for nodes with less than 50% that agreed with the consensus bootstrap tree. Branches without bootstrap designation are found in the maximum parsimony tree but not the bootstrap tree.

FIG. 5 shows bar graphs of the activity of S. pneumoniae neuraminidase. FIG. 5A respresents the titration of activity using concentrations of purified NanA as labeled. FIG. 5B demonstrates the effect of divalent cations on activity purified NanA relative to wild-type enzyme (control). Assay was performed using 2′-(4-methylumbelliferyl)-α-D-N-acetylneuraminic acid (MNN). *p-value<0.05.

FIG. 6 shows bar graphs of the inhibition of S. pneumoniae NanA neuraminidase activity by sialic acid compounds NANA and DANA. Activity is shown as a percentage of activity of NanA without inhibitor (control). Assay was performed using 2′-(4-methylumbelliferyl)-α-D-N-acetylneuraminic acid. *p-value<0.05.

FIG. 7 shows bar graphs of the release of sialic acid from the surface of airway epithelial cells by S. pneumoniae. FIG. 7A represents exposure of aGM1 by concentrated supernatant from wild-type and nanA strains. FIG. 7B shows exposure of aGM1 with purified NanA. Cells were stained with antibody to aGM1 and quantified by flow cytometry and are shown as the fold change compared to media only control. *p-value<0.05.

FIG. 8 shows graphs of biological activities of S. pneumoniae WT and nanA mutant. FIG. 8A demonstrates adherence to 16HBE airway epithelial cells. FIG. 8B depicts mouse colonization data. Mice were infected with 10⁵ cfu bacteria and cfu bacteria in the lung determined and Polymorphonuclear monocytes (PMNs) (FIG. 8C) were quantified by flow cytometry. For FIGS. 8B-C, n=5 for both groups, data not statistically significant (Mann Whitney non-parametric test).

FIG. 9 is a bar graph depicting S. pneumoniae biofilm formation. Encapsulated (D39 background) strains were grown in microtitre trays without (solid bars) or with (striped bars) previous epithelial cell exposure. Unencapsulated R6 strains were grown in microtitre trays without epithelial cell exposure. Biofilms were measured by crystal violet (CV) staining Representative experiments are shown * p-value<0.05.

FIG. 10 is a bar graph showing inhibition of neuraminidases by oseltamivir. Activity is expressed as a percentage of activity without inhibitor. Assay was performed using 2′-(4-methylumbelliferyl)-α-D-N-acetylneuraminic acid. *p-value<0.05.

FIG. 11 depicts graphs demonstrating inhibitory activity of candidate neuraminidase inhibitors. FIG. 11A is a graphing showing the screening of candidate inhibitors performed with NanA (black bars) and NanPs (gray bars) and inhibitors at 100 μM concentration in the neuraminidase assay. FIGS. 11B-C are dose response curves for NanPs and NanA neuraminidases with lead compound XX1. Data was fitted with a power-based trend line. Shown is percentage activity compared to the vehicle (DMSO) only control.

FIG. 12 are graphs that depict S. pneumoniae biofilm formation. FIG. 12A is a graph showing biofilm formation using encapsulated (D39 background) strains that were grown in microtitre trays without (solid bars) or with (striped bars) previous epithelial cell exposure. Unencapsulated R6 strains were grown in microtitre trays without epithelial cell exposure. FIG. 12B is a graph showing incubation with NANA results in reduced biofilm formation of the wild-type (D39) strain. Biofilms were measured by crystal violet (CV) staining Biofilm formation was normalized to growth and expressed as a percentage when compared to the R6 wild-type strain. * p-value<0.05.

FIG. 13 are photgraphs showing imaging of S. pneumoniae biofilms. FIG. 13A are images of CV stained biofilms in microtitre wells of WT and nanA strains in D39 (after epithelial cell exposure) and R6 backgrounds. FIG. 13B are photographic images of fluorescence microscopy of D39 WT and nanA biofilms grown in microtitre trays after epithelial cell exposure and stained with live/dead BacLight stain. Magnification was 200×. FIG. 13C is an image of a 3D reconstruction of biofilm structure seen with the WT strain in FIG. 13B. FIG. 13D is an image of a 3D reconstruction of cells seen with the nanA strain in FIG. 13B.

FIG. 14 depicts the inhibitory activity of NanA inhibitors. FIG. 14A is a graphs showing the screening of candidate inhibitors that was performed with NanA and inhibitors at 100 μM concentration in the neuraminidase assay. FIG. 14B is a dose response curve for NanA with lead compound XX1. Data was fitted with a logarithmic-based trend line. Shown is percentage activity compared to the vehicle (DMSO) only control. FIG. 14C is a bar graph that shows biofilm formation of wild-type D39 grown in the presence of XX1 during epithelial cell exposure and growth in microtitre trays. The nanA strain is shown as a reference. Biofilm formation was normalized to growth and expressed as a percentage when compared to the wild-type control. FIG. 14D is a schematic of the chemical structure of XX1. *p-value<0.05.

FIG. 15 is a schematic showing the synthesis of compounds of Formula I (Scheme 1)

FIG. 16 is a diagram of a synthetic scheme showing that compounds of the invention can be tautomerized (Scheme 2).

### DETAILED DESCRIPTION OF THE INVENTION

The invention is related to various methods for inhibiting biofilm formation, treating a biofilm production-related disorder, preventing biofilm formation, and screening for neuraminidase inhibitors. The invention also encompasses a mutant bacterial strain with a deletion in a neuraminidase gene.

### DEFINITIONS

All scientific and technical terms used in this application have meanings commonly used in the art unless otherwise specified. As used in this application, the following words or phrases have the meanings specified.

As used herein, the term “inhibitor of biofilm formation,” or “biofilm synthesis inhibitor” (such as a neuraminidase inhibitor) encompasses an agent that inhibits (e.g., disrupts) the attachment of microorganisms onto a surface, to the biofilm matrix itself, to other cells comprising the biofilm, or a combination thereof, and/or inhibits the ability of such microorganisms to produce, synthesize and/or accumulate biofilm on a surface.

The terms “disorder” and “disease” are used herein interchangeably for a condition in a subject. A disorder is a disturbance or derangement that affects the normal function of the body of a subject. A disease is a pathological condition of an organ, a body part, or a system resulting from various causes, such as infection, genetic defect, or environmental stress that is characterized by an identifiable group of symptoms. A disorder or disease can refer to a biofilm production-related disorder of the invention that is characterized by a disease-related growth of bacteria in that a biofilm is established.

The terms “prevent,” “preventing,” and “prevention” refer herein to the inhibition of the development or onset of a disorder or the prevention of the recurrence, onset, or development of one or more symptoms of a disorder in a subject resulting from the administration of a therapy (e.g., a prophylactic or therapeutic agent), or the administration of a combination of therapies (e.g., a combination of prophylactic or therapeutic agents).

As used herein, to “block” or “inhibit” a molecule, signal, or a receptor means to interfere with the binding of, or activation of the molecule, signal, or a receptor as detected by a test recognized in the art (such as binding assays). Blockage or inhibition can be partial or total, resulting in a reduction, increase, or modulation in the activation of the molecule, signal, or a receptor as detected by a test recognized in the art.

“Binding” refers to the interaction or association of a molecule with another entity, such as its target. This interaction can be covalent or noncovalent. The interaction of a molecule and its target site can be regulated by compositions of the invention. For example, administration of a neuraminidase inhibitor or a derivative thereof can block the action of its target, a neuraminidase.

As used herein, a “fragment” or “portion” is any part or segment of a molecule. For example, a fragment of a molecule includes that part that recognizes and binds its natural target. In the case of an antibody, the fragment is a binding portion of the whole antibody; in the case of a neuraminidase inhibitor, the fragment is that smaller portion of the entire inhibitor.

A “plumbing system” encompasses the faucets, valves, plumbing fixtures, piping (metal, plastic, and the like), water storage tanks, water recylcers, coils, bilges, hoses, tubing, and backflow preventers as well as their respective interior and exterior surfaces.

Aspects of the invention are related to methods of inhibiting biofilm formation. The method entails applying a neuraminidase inhibitor to the biofilm and measuring a reduction in the formation of a biofilm. The neuraminidase inhibitor modulates the activity or the expression of the neuraminidase (for example, a bacterial neuraminidase), thereby inhibiting biofilm formation.

Neuraminidases (sialidases) are produced by a wide variety of mucosal pathogens, ranging from S. pneumoniae in the airway to Vibrio cholerae in the gut (Vimr et al., (2004) Microbiol. Mol Biol. Rev. 68:132-153). While the central role of viral neuraminidase in pathogenesis of influenza is established (Colman (1994) Protein. Sci. 3:1687-1696) and provides a target for both vaccines and chemotherapy, the contribution of bacterial neuraminidase to the pathogenesis of infection is not as clearly defined. Neuraminidase producing species such as Hemophilus (Vimr et al., (2002) Trends. Microbiol. 10:254-257), Streptococcus pneumoniae (Camara et al., (1994) Infect. Immun. 62:3688-3695; King et al., (2004) Mol. Microbiol. 54:159-171), and P. aeruginosa (Cacalano et al., (1992) J. Clin. Invest. 89:1866-1874) share a common ecological niche, colonizing the heavily sialylated secretions and surfaces of the upper respiratory tract. Although each can bind to asialylated glycolipids exposed by neuraminidase activity (Krivan et al., (1988) Proc. Natl. Acad. Sci. U.S.A. 85:6157-6161), they differ substantially in their ability to either metabolize (Godoy et al., (1993) Infect. Immun. 61:4415-4426) or incorporate sialic acid into surface structures (Bouchet et al., (2003) Proc. Natl. Acad. Sci. U.S.A. 100:8898-8903). Thus, bacterial neuraminidases can interact with both microbial as well as eukaryotic glycoconjugates (Vimr et al., (2004) Microbiol. Mol. Biol. Rev. 68:132-153).

Viral neuraminidase inhibitors have been very useful in the prevention and treatment of influenza, targeting similar high-risk patient populations, such as those patients afflicted with pneumonia, CF, or chronic obstructive pulmonary disease (COPD). The NanPs neuraminidase, for example, of P. aeruginosa shares many conserved elements and folds in the manner predicted for other microbial neuraminidases (Roggentin et al., (1989) Glycoconj. J. 6:349-353; Rothe et al., (1991) Mol. Gen. Genet. 226:190-197).

P. aeruginosa (a Gram-negative bacterium) is a major opportunistic pathogen, an important cause of nosocomial pneumonia as well as the chief cause of lung infection in cystic fibrosis (CF), and is the most common lethal genetic disease of Caucasians. Over two decades ago, neuraminidase production in isolates of P. aeruginosa from CF patients was described and indicated to contribute to pulmonary infection (Leprat et al., (1980) Ann. Microbiol. (Paris) 131B:209-222).

Streptococcus pneumoniae, or pneumococcus, is a Gram-positive, diplococcus, alpha-hemolytic anaerobe that is a common inhabitant of the nasopharyngeal region. S. pneumoniae causes many types of infection other than pneumonia, including, but not limited to, meningitis, bacteremia (or septicaemia), acute sinusitis, otitis media, endocarditis, peritonitis, osteomyelitis, septic arthritis, pericarditis, cellulitis, and brain abscess. S. pneumoniae is the most common cause of bacterial meningitis in adults and children, and is one of the top two isolates found in ear infection, otitis media. Pneumococcal pneumonia is more common in the elderly and very young.

The capusle of S. pneumoniae is the most important element in its pathogenicity. Furthermore, nearly all S. pneumoniae clinical isolates have prominent sialidase activities. For example, the organism has been shown to encode 3 different neuraminidases: NanA, NanB, and NanC. For a further discussion, see Henriques-Normark et al., (2008) Nat Rev Microbiol. 6(11):827-37; and see Yao K H and Yang Y H., (2008) Vaccine, 26(35):4425-33.

The nucleotide sequence of S. pneumoniae neuraminidase (sialidase A; NanA) is shown in SEQ ID NO: 1. The polypeptide sequence of S. pneumoniae neuraminidase is depicted in SEQ ID NO:2. Sequence information related to NanA is accessible in public databases by GenBank Accession numbers NC_{—}008533 (for mRNA) and YP_{—}816960 (for protein).

SEQ ID NO: 1 is the S. pneumoniae wild type nucleotide sequence corresponding to the NanA (nucleotides 1-2994):

| | |
|---|---|
| 1 | atgattgtag gagcagtggt atttggaacg tctcctgttt tagctcaaga aggggcaagt |
| 61 | gagcaacctc tggcaaatga aactcaactt tcgggggaga gctcaaccct aactgataca |
| 121 | gaaaagagcc agccttcttc agagactgaa ctttctggca ataagcaaga acaagaaagg |
| 181 | aaagataagc aagaagaaaa aattccaaga gattactatg cacgagattt ggaaaatgtc |
| 241 | gaaacagtga tagaaaaaga agatgttgaa accaatgctt caaatggtca gagagttgat |
| 301 | ttatcaagtg aactagataa actaaagaaa cttgaaaacg caacagttca catggagttt |
| 361 | aagccagatg ccaaggcccc agcattctat aatctctttt ctgtgtcaag tgctactaaa |
| 421 | aaagatgagt acttcactat ggcagtttac aataatactg ctactctaga ggggcgtggt |
| 481 | tcggatggga aacagtttta caataattac aacgatgcac ccttaaaagt taaaccaggt |
| 541 | cagtggaatt ctgtgacttt cacagttgaa aaaccgacag cagaactacc taaaggccga |
| 601 | gtgcgcctct acgtaaacgg ggtattatct cgaacaagtc tgagatctgg caatttcatt |
| 661 | aaagatatgc cagatgtaac gcatgtgcaa atcggagcaa ccaagcgtgc caacaatacg |
| 721 | gtttgggggt caaatctaca gattcggaat ctcactgtgt ataatcgtgc tttaacacca |
| 781 | gaagaggtac aaaaacgtag tcaacttttt aaacgctcag atttagaaaa aaaactacct |
| 841 | gaaggagcgg ctttaacaga gaaaacggac atattcgaaa gcgggcgtaa cggtaaacca |
| 901 | aataaagatg gaatcaagag ttatcgtatt ccagcacttc tcaagacaga taaaggaact |
| 961 | ttgatcgcag gtgcagatga acgccgtctc cattcgagtg actggggtga tatcggtatg |
| 1021 | gtcatcagac gtagtgaaga taatggtaaa acttggggtg accgagtaac cattaccaac |
| 1081 | ttacgtgaca atccaaaagc ttctgaccca tcgatcggtt caccagtgaa tatcgatatg |
| 1141 | gtgttggttc aagatcctga aaccaaacga atcttttcta tctatgacat gttcccagaa |
| 1201 | gggaagggaa tctttggaat gtcttcacaa aaagaagaag cctacaaaaa aatcgatgga |
| 1261 | aaaacctatc aaatcctcta tcgtgaagga gaaaagggag cttataccat tcgagaaaat |
| 1321 | ggtactgtct atacaccaga tggtaaggcg acagactatc gcgttgttgt agatcctgtt |
| 1381 | aaaccagcct atagcgacaa gggggatcta tacaagggta accaattact aggcaatatc |
| 1441 | tacttcacaa caaacaaaac ttctccattt agaattgcca aggatagcta tctatggatg |
| 1501 | tcctacagtg atgacgacgg gaagacatgg tcagcgcctc aagatattac tccgatggtc |
| 1561 | aaagccgatt ggatgaaatt cttgggtgta ggtcctggaa caggaattgt acttcggaat |
| 1621 | gggcctcaca agggacggat tttgataccg gtttatacga ctaataatgt atctcactta |
| 1681 | aatggctcgc aatcttctcg tatcatctat tcagatgatc atggaaaaac ttggcatgct |
| 1741 | ggagaagcgg tcaacgataa ccgtcaggta gacggtcaaa agatccactc ttctacgatg |
| 1801 | aacaatagac gtgcgcaaaa tacagaatca acggtggtac aactaaacaa tggagatgtt |
| 1861 | aaactcttta tgcgtggttt gactggagat cttcaggttg ctacaagtaa agacggagga |
| 1921 | gtgacttggg agaaggatat caaacgttat ccacaggtta aagatgtcta tgttcaaatg |
| 1981 | tctgctatcc atacgatgca cgaaggaaaa gaatacatca tcctcagtaa tgcaggtgga |
| 2041 | ccgaaacgtg aaaatgggat ggtccacttg gcacgtgtcg aagaaaatgg tgagttgact |
| 2101 | tggctcaaac acaatccaat tcaaaaagga gagtttgcct ataattcgct ccaagaatta |
| 2161 | ggaaatgggg agtatggcat cttgtatgaa catactgaaa aaggacaaaa tgcctatacc |
| 2221 | ctatcattta gaaaatttaa ttgggacttt ttgagcaaag atctgatttc tcctaccgaa |
| 2281 | gcgaaagtga agcgaactag agagatgggc aaaggagtta ttggcttgga gttcgactca |
| 2341 | gaagtattgg tcaacaaggc tccaaccctt caattggcaa atggtaaaac agcacgcttc |
| 2401 | atgacccagt atgatacaaa aaccctccta tttacagtgg attcagagga tatgggtcaa |
| 2461 | aaagttacag gtttggcaga aggtgcaatt gaaagtatgc ataatttacc agtctctgtg |
| 2521 | gcgggcacta agctttcgaa tggaatgaac ggaagtgaag ctgctgttca tgaagtgcca |
| 2581 | gaatacacag gcccattagg gacatccggc gaagagccag ctccaacagt cgagaagcca |
| 2641 | gaatacacag gcccactagg gacatccggc gaagagccag ccccgacagt cgagaagcca |
| 2701 | gaatacacag gcccactagg gacagctggt gaagaagcag ctccaacagt cgagaagcca |
| 2761 | gaatttacag ggggagttaa tggtacagag ccagctgttc atgaaatcgc agagtataag |
| 2821 | ggatctgatt cgcttgtaac tcttactaca aaagaagatt atacttacaa agctcctctt |
| 2881 | gctcagcagg cacttcctga aacaggaaac aaggagagtg acctcctagc ttcactagga |
| 2941 | ctaacagctt tcttccttgg tctgtttacg ctagggaaaa agagagaaca ataa |

SEQ ID NO: 2 is the S. pneumoniae wild type amino acid sequence corresponding to NanA (residues 1-997):

MIVGAVVFGTSPVLAQEGASEQPLANETQLSGESSTLTDTEKSQPSSETELSGNKQEQ
ERKDKQEEKIPRDYYARDLENVETVIEKEDVETNASNGQRVDLSSELDKLKKLENAT
VHMEFKPDAKAPAFYNLFSVSSATKKDEYFTMAVYNNTATLEGRGSDGKQFYNNY
NDAPLKVKPGQWNSVTFTVEKPTAELPKGRVRLYVNGVLSRTSLRSGNFIKDMPDV
THVQIGATKRANNTVWGSNLQIRNLTVYNRALTPEEVQKRSQLFKRSDLEKKLPEG
AALTEKTDIFESGRNGKPNKDGIKSYRIPALLKTDKGTLIAGADERRLHSSDWGDIGM
VIRRSEDNGKTWGDRVTITNLRDNPKASDPSIGSPVNIDMVLVQDPETKRIFSIYDMF
PEGKGIFGMSSQKEEAYKKIDGKTYQILYREGEKGAYTIRENGTVYTPDGKATDYRV
VVDPVKPAYSDKGDLYKGNQLLGNIYFTTNKTSPFRIAKDSYLWMSYSDDDGKTWS
APQDITPMVKADWMKFLGVGPGTGIVLRNGPHKGRILIPVYTTNNVSHLNGSQSSRII
YSDDHGKTWHAGEAVNDNRQVDGQKIHSSTMNNRRAQNTESTVVQLNNGDVKLF
MRGLTGDLQVATSKDGGVTWEKDIKRYPQVKDVYVQMSAIHTMHEGKEYIILSNA
GGPKRENGMVHLARVEENGELTWLKHNPIQKGEFAYNSLQELGNGEYGILYEHTEK
GQNAYTLSFRKFNWDFLSKDLISPTEAKVKRTREMGKGVIGLEFDSEVLVNKAPTLQ
LANGKTARFMTQYDTKTLLFTVDSEDMGQKVTGLAEGAIESMHNLPVSVAGTKLSN
GMNGSEAAVHEVPEYTGPLGTSGEEPAPTVEKPEYTGPLGTSGEEPAPTVEKPEYTG
PLGTAGEEAAPTVEKPEFTGGVNGTEPAVHEIAEYKGSDSLVTLTTKEDYTYKAPLA
QQALPETGNKESDLLASLGLTAFFLGLFTLGKKREQ

Many pulmonary pathogens, including P. aeruginosa, bind to the GalNAcβ1-4Gal moiety exposed on asialylated glycolipids (Krivan et al., (1988) Proc. Natl. Acad. Sci. U.S.A. 85:6157-6161). Therefore, the ability to de-sialylate mucosal surfaces can contribute to bacterial colonization of the airways. The P. aeruginosa neuraminidase was shown to be osmo-regulated, and accordingly, to be consistent with expression in the milieu of the CF lung (Cacalano et al., (1992) J. Clin. Invest. 89:1866-1874). This neuraminidase is capable of exposing the receptor asialoganglioside gangliotetraosylceramide (asialoGM1) (Galβ1,2GalNAcβ1,4Galβ1,4Glcβ1,1Cer) on the surface of CF airway cells in vitro (Saiman et al., (1993) J. Clin. Invest. 92:1875-1880). However, data to document P. aeruginosa adherence to the airway surface in CF patients has been lacking (Baltimore et al., (1989) Am. Rev. Respir. Dis. 140:1650-1661). The current consensus is that organisms are predominantly entrapped in dehydrated secretions of the lung and by shedding proinflammatory products activate airway inflammation, a model that does not require direct attachment of organisms to the epithelial surface (Boucher (2004) Eur. Respir. J. 23:146-158). Nonetheless, analyses of P. aeruginosa gene expression in CF patients document that the NanPs (also referred to as PA2794) neuraminidase locus is one of the most highly expressed genes in this patient population in vivo (Lanotte et al., (2004) J. Med. Microbiol. 53:73-81). Unlike other respiratory pathogens, P. aeruginosa cannot use sialic acid as a carbon source nor does it contain sialic acid as a component of its LPS (Knirel et al., (1988) Acta. Microbiol. Hung. 35:3-24).

Gram-negative bacteria and Gram-positive bacteria, in addition to other unicellular organisms, can produce biofilms. Bacterial biofilms are surface-attached communities of cells that are encased within an extracellular polysaccharide matrix produced by the colonizing cells. Biofilm development occurs via a series of programmed steps, which include an initial attachment to a surface, formation of three-dimensional microcolonies, and the subsequent development of a mature biofilm. Biofilms can be composed of various microorganisms (such as viruses, bacteria, protozoa, and fungi) co-existing within the community and a particular cellular type can predominate. The more deeply a cell is located within a biofilm (such as, the closer the cell is to the solid surface to which the biofilm is attached to, thus being more shielded and protected by the bulk of the biofilm matrix), the more metabolically inactive the cells are. The consequences of this physiologic variation and gradient create a collection of bacterial communities where there is an efficient system established whereby microorganisms have diverse functional traits. A biofilm also is made up of various and diverse non-cellular components and can include, but are not limited to carbohydrates (simple and complex), lipids, proteins (including polypeptides), and lipid complexes of sugars and proteins (lipopolysaccharides and lipoproteins).

Bacterial biofilms exist in nature as well as in medical and industrial environments, such as a GMP facility. The biofilm can allow bacteria to exist in a dormant state for a certain amount of time until suitable growth conditions arise thus offering the microorganism a selective advantage to ensure its survival. However, this selection can pose serious threats to human health in that biofilms have been observed to be involved in about 65% of human bacterial infections (Smith (2005) Adv. Drug Deliv. Rev. 57:1539-1550; Hall-Stoodley et al., (2004) Nat. Rev. Microbiol. 2: 95-108). In fact, the majority of infections that occur in animals are biofilm-based. Biofilms are problematic with respect to respiratory conditions and diseases. Cystic Fibrosis (CF), one of the most common fatal genetic disorders in the United States, is most prevalent in Caucasians. It occurs on an average of one in every 3,300 live births, and causes the death of patients inflicted with CF by the age of 30. A mutation in a gene that encodes a chloride transport channel produces partially functional or completely dysfunctional transport channels. CF patients develop thick mucus secretions, which result from disruption of physiological salt/water balance due to the defective transport channel. The secretions clog bronchial tubes in the lungs and can additionally block exit passages of the pancreas and intestines, which lead to loss of function of these organs.

The mucus secretions are depleted of oxygen due to the metabolic activity of neutrophils, aerobic bacteria, and even epithelial cells. Within this mucus, P. aeruginosa, for example, is found to thrive. P. aeruginosa also is an important cause of nosocomial pneumonia. It infects the elderly, cancer chemotherapy patients, and immuno-compromised individuals.

Other medical conditions and treatments resulting in the development of undesirable biofilms include, but are not limited to, medical device-related infections, catheter-related infection (kidney, vascular, peritoneal), chronic otitis media, prostatitis, dental caries, wounds, acne, chronic obstructive pulmonary disease, infectious kidney stones, orthopedic implant infection, cystitis, bronchiectasis, bacterial endocarditis, Legionnaire's disease, osteomyelitis, and biliary stents (see US Appln. Pub. No. 20050158253). Thus, there is a need in the art for improved therapeutic approaches for the inhibition of biofilm formation and/or the reduction or elimination of biofilms.

Harsh treatments (such as chemicals and abrasives) have been used to reduce, prevent, or control biofilm formation. However, biological environments (for example, airways, the urinary tract, wound sites, etc) are sensitive to such harsh treatments. Thus, better methods are needed to control biofilm formation.

In industrial settings, biofilms (comprised of viruses, bacteria, protozoa, fungi, and the like) can adhere to surfaces, such as pipes and filters. Biofilms are problematic in industrial settings because they cause biocorrosion and biofouling in industrial systems, such as heat exchangers, oil pipelines, water systems, filters, and the like (Coetser et al., (2005) Crit. Rev. Micro. 31: 212-32). Thus, biofilms can inhibit fluid flow-through in pipes, clog water and other fluid systems, as well as serve as reservoirs for pathogenic bacteria, protozoa, and fungi. As such, industrial biofilms are an important cause of economic inefficiency in industrial processing systems.

Biofilms (also referred to as “slime residues”) can affect a wide variety of commercial, industrial, and processing operations (such as Good Manufacturing Practices (GMP) facilities). Since biofilms are ubiquitous in water handling systems, S. pneumoniae a gram-positive, ovoid bacterium (and/or other bacteria, protozoa, fungi and some viruses) can be associated with these biofilms. In many instances, S. pneumoniae is a substantial microbial component. Thus, there is a need for compositions and methods for controlling biofilms in commercial settings as well as biological environments.

The biofilm to be inhibited can be harbored by a subject, can be in vitro, or can be on the surface of an implantable/insertable device to be inserted into a subject.

For example, the terms can refer to a mammal including, but not limited to, and a primate (e.g., a monkey, such as a cynomolgous monkey, a chimpanzee, and a human). For example, the subject can be a non-human animal such as a bird (e.g., a quail, chicken, or turkey), a farm animal (e.g., a cow, goat, horse, pig, or sheep), a pet (e.g., a cat, dog, or guinea pig, rat, or mouse), or laboratory animal (e.g., an animal model for a disorder). The subject according to the invention is a human (e.g., an infant, child, adult, or senior citizen).

For example, the subject according to the invention can be an animal, such as a mammal. The mammal can be a non-primate (for example, a cow, pig, bird, sheep, goat, horse, cat, dog, rat, rabbit, mouse, and the like) or a primate (for example, a monkey, such as a cynomolgous monkey, a chimpanzee, a human). Non-limiting representative subjects according to the invention can be a human infant, a pre-adolescent child, an adolescent, an adult, or a senior/elderly adult.

A neuraminidase is an enzyme protein (for example, bacterial, viral, and the like) that cleaves terminal sialic acid residues from carbohydrate moieties on the surfaces of cells infected with such pathogens (for example, bacteria or viruses). This cleavage can result in the release of progeny pathogens from infected cells. Thus, administration of neuraminidase inhibitors can serve as a treatment that limits the severity and spread of pathogenic infections. The neuraminidase inhibitor can also modulate the expression of a neuraminidase via reducing the expression of the neuraminidase. The modulation of neuraminidase activity and/or expression (for example, its reduction) can be due to decreased transcription and/or translation of the neuraminidase molecule, which results in reduced amounts of neuraminidase synthesized by the cell.

Initial studies of the P. aeruginosa neuraminidase performed with purified enzyme, and in vitro analyses were consistent with a role for the enzyme in modifying airway epithelial cell surfaces to facilitate bacterial attachment (Cacalano et al., (1992) J. Clin. Invest. 89:1866-1874). Moreover, as CF airways were more readily modified than were normal airway cells, the Pseudomonas enzyme seemed to be important in that disease (Saiman et al., (1993) J. Clin. Invest. 92:1875-1880). However, it has been determined with tests performed under more physiological conditions in vivo using isogenic mutants that the P. aeruginosa neuraminidase has a different function. The neuraminidase, in addition to other bacterial neuraminidases, is important for biofilm production, as well as the cell-cell interactions that were critical in the initial colonization process. Recent studies indicate that there are significant homologies between the genes involved in sialic acid O-acetylation in many bacterial species (Lewis et al., (2006) J. Biol. Chem. 281:11186-11192). Just as autolysins are necessary for cell wall biosynthesis, enzymes that cleave carbohydrate linkages are necessary for the growth and modification of extracellular polysaccharides during biofilm biosynthesis (Vuong et al., (2004) J. Biol. Chem. 279:54881-54886). The invention provides for methods for inhibiting or reducing biofilm formation using neuraminidase inhibitors.

A neuraminidase inhibitor according to the invention can be used to inhibit the formation of a biofilm by any biofilm-forming organism, such as viruses, bacteria, protozoa, and fungi. Biofilms are comprised of various microorganisms, such as viruses, bacteria, protozoa, and fungi, (e.g., Borrelia sp., Streptococcus sp., Neisseria sp., Pseudomonas sp., Haemophilus sp., Vibrio sp., Bacillus sp., Klebsiella sp., Burkholderia sp., Salmonella sp., Legionella sp., P. aeruginosa, H. influenzae, V. cholerae, Yersinia pestis, Escherichia coli, Streptococcus pneumoniae, Proteus mirablis, and Francisella tularensis) and can be found in a live subject, in vitro, or on a surface, such as on or in the pipes of a plumbing system or industrial equipment.

The neuraminidase inhibitor to be used to inhibit biofilm formation in the method of the invention can be any compound, small molecule, peptide, protein, aptamer, ribozyme, RNAi, or antisense oligonucleotide and the like.

For example, a neuraminidase inhibitor according to the invention can be a protein, such as an antibody (monoclonal, polyclonal, humanized, and the like), or a binding fragment thereof, directed against a neuraminidase protein, such as a viral, protozoan, fungal, or bacterial neuraminidase (such as S. pneumoniae, H. influenzae, or V. cholerae). An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing).

Additionally, a neuraminidase inhibitor can be a non-antibody peptide or polypeptide that binds to a bacterial neuraminidase. A peptide or polypeptide can be a portion of a protein molecule of interest other than the full-length form, and includes peptides that are smaller constituents that exist within the full-length amino acid sequence of a protein molecule of interest. These peptides can be obtained commercially or synthesized via liquid phase or solid phase synthesis methods (Atherton et al., (1989) Solid Phase Peptide Synthesis: a Practical Approach. IRL Press, Oxford, England). For example, the neuraminidase inhibitor can be a peptide that interacts with a Streptococcus neuraminidase, such as the protein encoded by the NanA gene (e.g., a protein comprising the amino acid sequence of SEQ ID NO:2). The peptide or protein-related neuraminidase inhibitors can be isolated from a natural source, genetically engineered or chemically prepared. These methods are well known in the art.

A neuraminidase inhibitor can also be a small molecule that binds to a neuraminidase and disrupts its function. Small molecules are a diverse group of synthetic and natural substances generally having low molecular weights. They are isolated from natural sources (for example, plants, fungi, microbes and the like), are obtained commercially and/or available as libraries or collections, or synthesized. Candidate neuramindase inhibitor small molecules can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries as described below (Werner et al., (2006) Brief Funct. Genomic Proteomic 5(1):32-6). In one embodiment, the neuraminidase inhibitor is a compound of the Formula I:

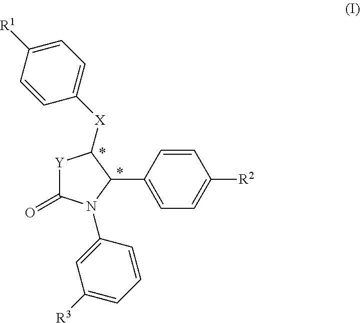

wherein,

R¹ is H, halogen, cyano, azido, nitro, C₁-C₆ alkyl, or C₁-C₆ alkoxy;

R² is H, halogen, cyano, azido, nitro, C₁-C₆ alkyl, or C₁-C₆ alkoxy;

R³ is H, —CO₂R⁴ or —CON(R⁴)₂;

each R⁴ is, independently, H or C₁-C₆ alkyl;

X is —CH₂—, —(C═O)—, —(C═NH)—, —(C═N—O—C₁-C₆-alkyl)-, or —(C═S)—; and

Y is —(C═O)—, —(C═NH)—, —(C═N—O—C₁-C₆-alkyl)-, or —(C═S)—,

or a pharmaceutically acceptable salt or hydrate thereof.

In one embodiment, R¹ is a halogen. In a specific embodiment, R¹ is chlorine.

In one embodiment, R² is a C₁-C₆ alkoxy group. In specific embodiments, R² is methoxy or ethoxy. In a specific embodiment, R² is methoxy.

In one embodiment, R³ is —CONH₂. In another embodiment, R³ is —CO₂H.

In one embodiment, X is —(C═NH)—, —(C═NOH)—, —(C═NOMe)-, —(C═O)—, or —C(═S)—. In a specific embodiment, X is —(C═O)—.

In one embodiment, Y is —(C═NH)—, —(C═NOH)—, —(C═NOMe)-, —(C═O)—, or —C(═S)—. In a specific embodiment, Y is —(C═O)—.

In one embodiment, R¹, R², and R³ are not H.

Pharmaceutically acceptable salts are known in the art, and may be selected from those listed in Berge, et al. [“Pharmaceutical Salts,” J. Pharm. Sci., 66(1):1-19 (January 1977)]. In one embodiment, a pharmaceutically acceptable salt of a compound of Formula (I) is an acid addition salt, for example a hydrochloride, sulfate, or phosphate salt. In another embodiment, a pharmaceutically acceptable salt of a compound of Formula (I) is a base addition salt, for example a sodium, potassium, calcium, or ammonium salt. In another embodiment, the base addition salt is a tetrafluoroboro salt.

In one embodiment, a compound of Formula (I) is a zwitterion.

In one embodiment, the chiral centers marked by asterix in Formula (I) are both R.

In one embodiment, a Compound of Formula (I) is Compound A:

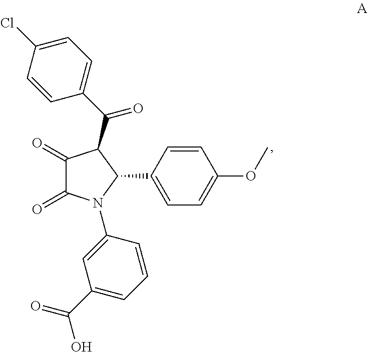

or a pharmaceutically acceptable salt or hydrate thereof.

Compound A is alternatively known by the chemical name, 3-((2R,3R)-3-(4-chlorobenzoyl)-2-(4-methoxyphenyl)-4,5-dioxopyrrolidin-1-yl)benzoic acid.

In some embodiments, the neuraminidase inhibitor is a compound comprising Formula (X):

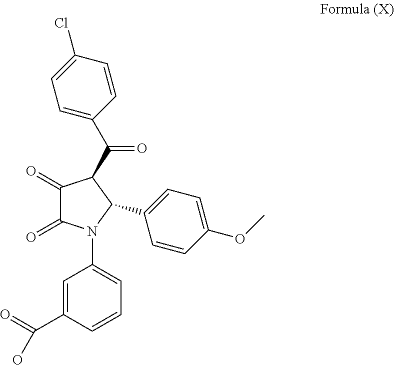

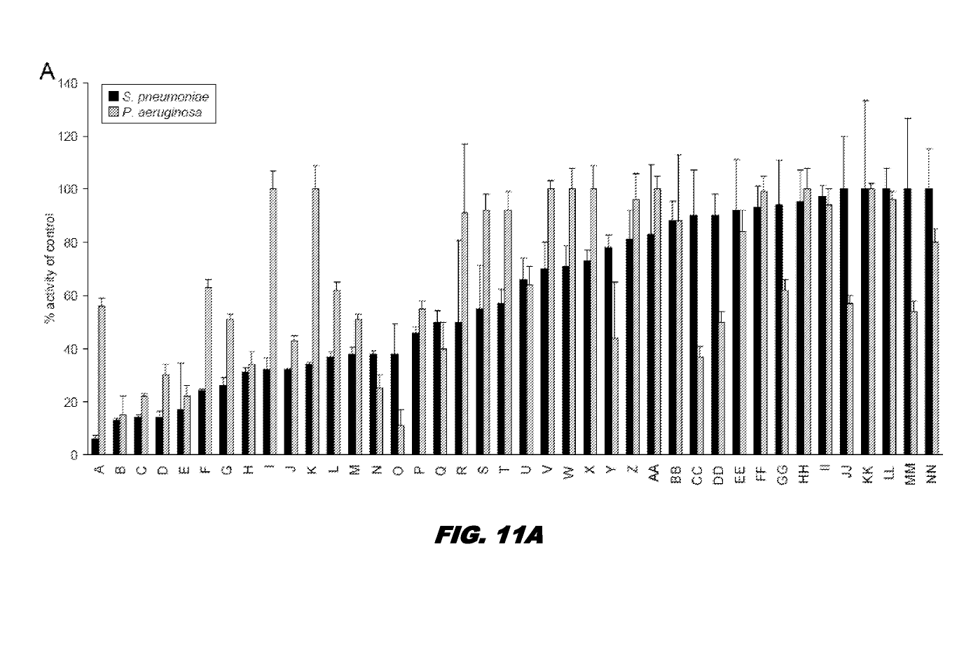

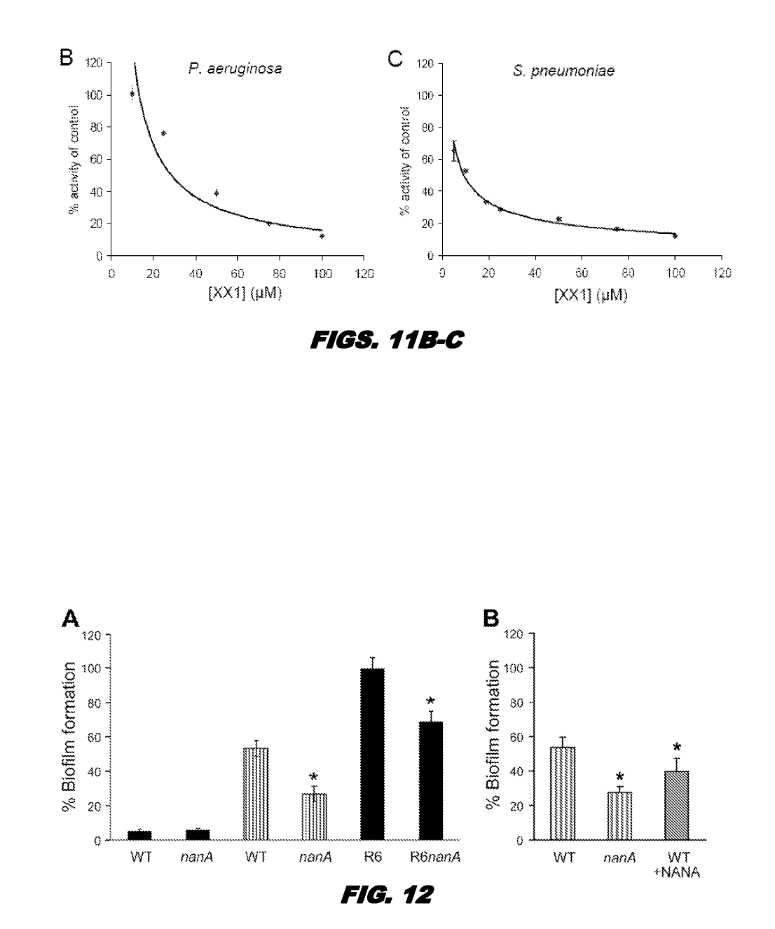

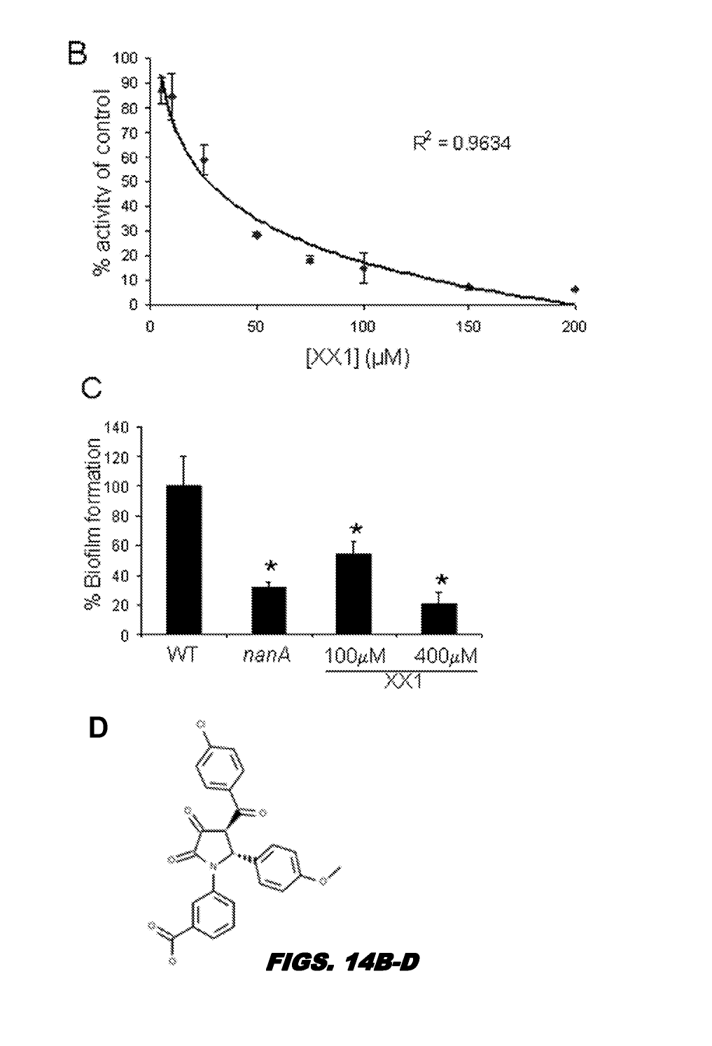

In specific embodiments, the compound comprising Formula (X) is compound XX1 (see Examples 1 and 3, and FIGS. 11 and 14). The compound of XX1 is available from Schrödinger LLC, (Portland, Oreg., USA).

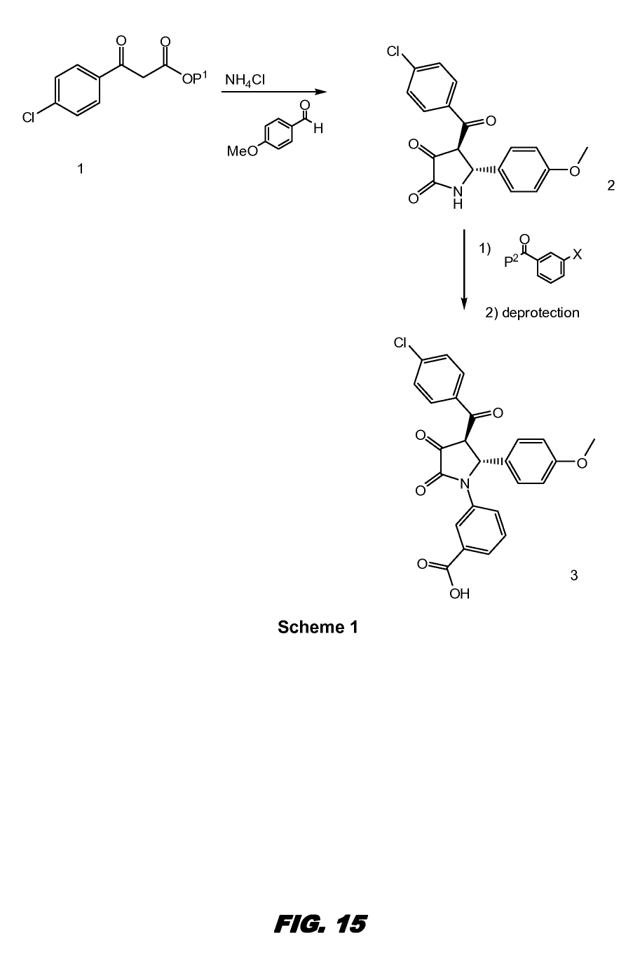

Compounds of Formula I can be made by methods known in the art, such as that in Scheme 1 (see FIG. 15).

Compounds of Formula I can be made by protecting a commercially-available benzyl maleate derivative, for example using the chlorobenzyl derivative to yield compound 3. Protecting group P¹ can be groups capable of forming an amide with amines, for examples esters such as methyl, or ethyl, or others suitable to accomplish the ring closure yielding compound 2. The ketones of resultant pyrrolidone derivative, e.g. 2, can be protected from the nucleophilic aromatic substitution reaction, for example as oxolanes, using standard techniques. Next, the benzoic acid group can be installed using nucleophilic aromatic substitution techniques on a suitably protected iodo-benzoic acid derivative. The nucleophilic aromatic substitution conditions can use an activating agent such as a metal cation complex as known in the art. Alternatively, the aromatic substitution can be accomplished with activated benzoic acid derivatives using a Stille coupling, a Suzuki cross-coupling, or a Buchwald-Hartwig cross-coupling. The substituent “X” is selected according to the coupling reaction conditions chosen. In one example of Stille conditions, X is SnR₃, such as SnBu₃. X can also be OTf, I, or B(OR)₃, where R is lower alkyl. The carboxylate and ketone groups can be protected before the nucleophilic aromatic substitution step as taught in Greene's Protective Groups in Organic Synthesis, 4^{th} Ed. Wuts and Greene, Eds. Wiley, 2007, which is incorporated herein by reference in its entirety. The product compound, e.g. 3, can be achiral or racemic. If such is the case, the chiral version can be obtained by chiral HPLC purification using techniques known to one skilled in the art.

Standard protection and deprotection techniques are known to one skilled in the art, and taught in Greene's Protective Groups in Organic Synthesis, 4^{th} Ed. Wuts and Greene, Eds. Wiley, 2007. Solvent and reaction conditions can be chosen as apparent to one skilled in the art in order to accomplish the depicted synthetic transformations. Such conditions can also be found in March, Advanced Organic Chemistry, 4^{th} Ed. Wiley (1992) and references cited therein, which is incorporated herein by reference in its entirety.

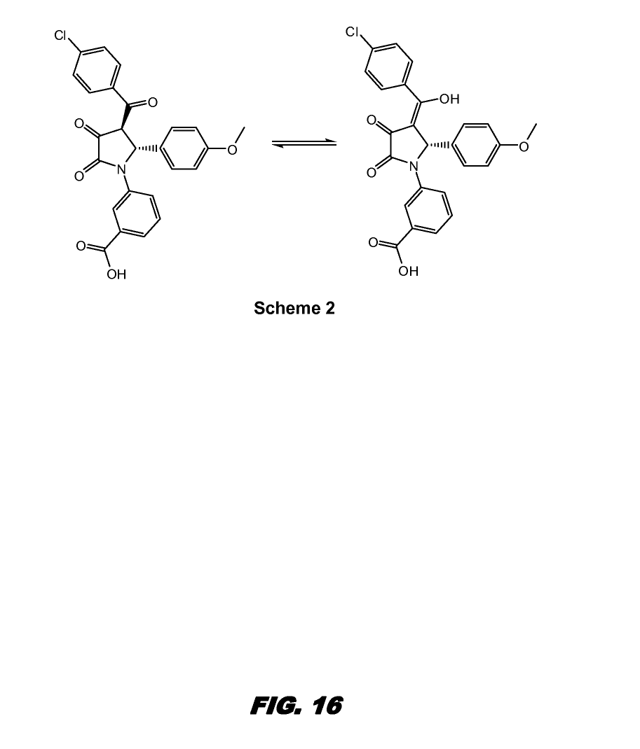

Compounds of the invention may also tautomerize, for example, as shown in Scheme 2 (see FIG. 16).

According to this invention, the neuraminidase inhibitor can also be an FDA approved viral neuraminidase inhibitor, such as the viral neuraminidase inhibitor oseltamivir (Tamiflu), zanamivir (Relenza; Glaxo Smith Kline, Research Triangle Park, NC), peramivir (BioCryst, Birmingham, Ala.), or a variant thereof. For example, the viral neuraminidase inhibitor, oseltamivir is an ethyl ester prodrug that can be purchased from Roche Laboratories (Nutley, N.J.). Amino acid sequences of FDA approved viral neuraminidase inhibitors can also be derivatized, for example, bearing modifications other than insertion, deletion, or substitution of amino acid residues, thus resulting in a variation of the original product (a variant). These modifications can be covalent in nature, and include for example, chemical bonding with lipids, other organic moieties, inorganic moieties, and polymers. For reviews on viral neuraminidase inhibitors, please see Klumpp et al., (2006) Curr. Top. Med. Chem. 6(5):423-34; Zhang et al., (2006) Mini Rev. Med. Chem. 6(4):429-48; Jefferson et al., (2006) Lancet 367(9507):303-13; Alymova et al., (2005) Curr Drug Targets Infect. Disord. 5(4):401-9; Moscona (2005) N. Engl. J. Med. 353(13):1363-73; De Clercq (2004) J. Clin. Virol. 30(2):115-33; Stiver (2003) CMAJ 168(1):49-56; Oxford et al., (2003) Expert Rev. Anti. Infect. Ther. 1(2):337-42; Cheer et al., (2002) Am. J. Respir. Med. 1(2):147-52; Sidewell et al., (2002) Expert Opin. Investig. Drugs. 11(6):859-69; Doucette et al., (2001) Expert Opin. Pharmacother. 2(10):1671-83; Young et al., (2001) Philos. Trans. R. Soc. Lond. B. Biol. Sci. 356(1416):1905-13; Lew et al., (2000) Curr. Med. Chem. 7(6):663-72); Taylor et al., (1996) Curr. Opin. Struct. Biol. 1996 6(6):830-7; and U.S. Patent Appln. Nos. 20060057658 and 20040062801.

Inhibition of RNA can effectively inhibit expression of a gene from which the RNA is transcribed. Inhibitors are selected from the group comprising: siRNA, interfering RNA or RNAi; dsRNA; RNA Polymerase III transcribed DNAs; ribozymes; and antisense nucleic acid, which can be RNA, DNA, or artificial nucleic acid. Also within the scope of the present invention are oligonucleotide sequences that include antisense oligonucleotides and ribozymes that function to bind to, degrade and/or inhibit the translation of an mRNA encoding a neuraminidase, such as a bacterial neuraminidase.

Antisense oligonucleotides, including antisense DNA, RNA, and DNA/RNA molecules, act to directly block the translation of mRNA by binding to targeted mRNA and preventing protein translation. For example, antisense oligonucleotides of at least about 15 bases and complementary to unique regions of the DNA sequence encoding a neuraminidase polypeptide can be synthesized, e.g., by conventional phosphodiester techniques (Dallas et al., (2006) Med. Sci. Monit. 12(4):RA67-74; Kalota et al., (2006) Handb. Exp. Pharmacol. 173:173-96; Lutzelburger et al., (2006) Handb. Exp. Pharmacol. 173:243-59).

siRNA comprises a double stranded structure containing 15 to 50 base pairs, and having a nucleotide sequence identical or nearly identical to an expressed target gene or RNA within the cell. In one embodiment, an siRNA comprises a double stranded structure containing 21 to 25 base pairs. Antisense polynucleotides include, but are not limited to: morpholinos, 2′-O-methyl polynucleotides, DNA, RNA and the like. RNA polymerase III transcribed DNAs contain promoters, such as the U6 promoter. These DNAs can be transcribed to produce small hairpin RNAs in the cell that can function as siRNA or linear RNAs that can function as antisense RNA. The inhibitor can be polymerized in vitro, recombinant RNA, contain chimeric sequences, or derivatives of these groups. The inhibitor can contain ribonucleotides, deoxyribonucleotides, synthetic nucleotides, or any suitable combination such that the target RNA and/or gene is inhibited. In addition, these forms of nucleic acid can be single, double, triple, or quadruple stranded. (see for example Bass (2001) Nature, 411, 428 429; Elbashir et al., (2001) Nature, 411, 494 498; and PCT Publication Nos. WO 00/44895, WO 01/36646, WO 99/32619, WO 00/01846, WO 01/29058, WO 99/07409, WO 00/44914).

Ribozymes are enzymatic RNA molecules that catalyze the specific cleavage of RNA. The mechanism of ribozyme action involves sequence specific hybridization of the ribozyme molecule to complementary target RNA encoding the neuraminidase, followed by endonucleolytic cleavage. Engineered hammerhead motif ribozyme molecules that specifically and efficiently catalyze endonucleolytic cleavage of mRNA sequences encoding a neuraminidase inhibitor, such as a bacterial neuraminidase inhibitor, are also within the scope of the present invention. Scanning the target molecule for ribozyme cleavage sites that include the following sequences, GUA, GUU, and GUC initially identifies specific ribozyme cleavage sites within any potential RNA target. Once identified, short RNA sequences of between about 15 and 20 ribonucleotides corresponding to the region of the target gene containing the cleavage site can be evaluated for predicted structural features such as secondary structure that can render the oligonucleotide sequence unsuitable. The suitability of candidate targets can also be evaluated by testing their accessibility to hybridization with complementary oligonucleotides using, e.g., ribonuclease protection assays.

Both the antisense oligonucleotides and ribozymes of the present invention can be prepared by known methods. These include techniques for chemical synthesis such as, e.g., by solid phase phosphoamite chemical synthesis. Alternatively, antisense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters.

Various modifications to the oligonucleotides of the present invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

An aptamer can be nucleic acid ligand that, through its ability to adopt a specific three-dimensional conformation, binds to and has an antagonizing (i.e., inhibitory) effect on a target. The target of the present invention is neuraminidase, and hence the term neuraminidase aptamer or nucleic acid ligand or neuraminidase aptamer or nucleic acid ligand is used. Inhibition of the target by the aptamer can occur by binding of the target, by catalytically altering the target, by reacting with the target in a way which modifies/alters the target or the functional activity of the target, by covalently attaching to the target as in a suicide inhibitor, by facilitating the reaction between the target and another molecule. Aptamers can be comprised of multiple ribonucleotide units, deoxyribonucleotide units, or a mixture of both types of nucleotide residues. Aptamers can further comprise one or more modified bases, sugars or phosphate backbone units as described in further detail herein.

Aptamers nucleic acid sequences are readily made that bind to a wide variety of target molecules. The aptamer nucleic acid sequences of the invention can be comprised entirely of RNA or partially of RNA, or entirely or partially of DNA and/or other nucleotide analogs. Aptamers are developed to bind particular ligands by employing known in vivo or in vitro (more often, in vitro) selection techniques known as SELEX (Systematic Evolution of Ligands by Exponential Enrichment). Methods of making aptamers are described in, for example, Ellington and Szostak (1990) Nature 346:818, Tuerk and Gold (1990) Science 249:505, U.S. Pat. No. 5,582,981; PCT Publication No. WO 00/20040; U.S. Pat. No. 5,270,163; Lorsch and Szostak (1994) Biochem. 33:973; Mannironi et al., (1997) Biochem. 36:9726; Blind (1999) Proc. Nat'l. Acad. Sci. USA 96:3606-3610; Huizenga and Szostak (1995) Biochem. 34:656-665; PCT Publication Nos. WO 99/54506, WO 99/27133, and WO 97/42317; and U.S. Pat. No. 5,756,291.

Generally, in their most basic form, in vitro selection techniques for identifying RNA aptamers involve first preparing a large pool of DNA molecules of the desired length that contain at least some region that is randomized or mutagenized. For instance, a common oligonucleotide pool for aptamer selection can contain a region of 20-100 randomized nucleotides flanked on both ends by an about 15-25 nucleotide long region of defined sequence useful for the binding of PCR primers. The oligonucleotide pool is amplified using standard PCR techniques. The DNA pool is then transcribed in vitro. The RNA transcripts are then subjected to affinity chromatography. The transcripts are passed through a column or contacted with magnetic beads or the like on which the target ligand has been immobilized. RNA molecules in the pool, which bind to the ligand, are retained on the column or bead, while nonbinding sequences are washed away. The RNA molecules, which bind the ligand, are then reverse transcribed and amplified again by PCR (usually after elution). The selected pool sequences are then put through another round of the same type of selection. The pool sequences are put through a total of about three to ten iterative rounds of the selection procedure. The cDNA is then amplified, cloned, and sequenced using standard procedures to identify the sequence of the RNA molecules that act as aptamers for the target ligand.

One can generally choose a suitable ligand without reference to whether an aptamer is yet available. In most cases, an aptamer can be obtained which binds the small, organic molecule of choice by someone of ordinary skill in the art. The unique nature of the in vitro selection process allows for the isolation of a suitable aptamer that binds a desired ligand despite a complete dearth of prior knowledge as to what type of structure can bind the desired ligand.

The association constant for the aptamer and associated ligand is, for example, such that the ligand functions to bind to the aptamer and have the desired effect at the concentration of ligand obtained upon administration of the ligand. For in vivo use, for example, the association constant should be such that binding occurs below the concentration of ligand that can be achieved in the serum or other tissue (such as ocular vitreous fluid). For example, the required ligand concentration for in vivo use can have undesired effects on the organism.

The aptamer nucleic acid sequences, in addition to including RNA, DNA and mixed compositions, can be modified. For example, certain modified nucleotides can confer improved characteristic on high-affinity nucleic acid ligands containing them, such as improved in vivo stability or improved delivery characteristics. Examples of such modifications include chemical substitutions at the ribose and/or phosphate and/or base positions. SELEX-identified nucleic acid ligands containing modified nucleotides are described in U.S. Pat. No. 5,660,985, entitled “High Affinity Nucleic Acid Ligands Containing Modified Nucleotides,” that describes oligonucleotides containing nucleotide derivatives chemically modified at the 5- and 2′-positions of pyrimidines. U.S. Pat. No. 5,637,459, supra, describes highly specific nucleic acid ligands containing one or more nucleotides modified with 2′-amino (2′—NH.sub.2), 2′-fluoro (2′-F), and/or 2′-O-methyl (2′-OMe). U.S. application Ser. No. 08/264,029, filed Jun. 22, 1994, entitled “Novel Method of Preparation of Known and Novel 2′ Modified Nucleosides by Intramolecular Nucleophilic Displacement,” describes oligonucleotides containing various 2′-modified pyrimidines.

The aptamer nucleic acid sequences of the invention further can be combined with other selected oligonucleotides and/or non-oligonucleotide functional units as described in U.S. Pat. No. 5,637,459, entitled “Systematic Evolution of Ligands by Exponential Enrichment: Chimeric SELEX,” and U.S. Pat. No. 5,683,867, entitled “Systematic Evolution of Ligands by Exponential Enrichment: Blended SELEX,” respectively.

Diversity libraries, such as random or combinatorial peptide or non-peptide libraries can be screened for small molecules and compounds that specifically bind to a bacterial, viral, yeast, or protozoan neuraminidase. Many libraries are known in the art that can be used such as, e.g., chemically synthesized libraries, recombinant (e.g., phage display) libraries, and in vitro translation-based libraries.

Any screening technique known in the art can be used to screen for agonist or antagonist molecules (such as neuraminidase inhibitors) directed at a target of interest (e.g. a neuraminidase, such as a bacterial neuraminidase). The present invention encompasses screens for small molecule ligands or ligand analogs and mimics, as well as screens for natural ligands that bind to and antagonize neuraminidase inhibitors, such as via examining the degree of biofilm inhibition utilizing previously described biofilm assays. For example, natural products libraries can be screened using assays of the invention for molecules that agonize or antagonize the activity of a molecule of interest, such as a neuraminidase.

Knowledge of the primary sequence of a molecule of interest, such as a neuraminidase inhibitor, and the similarity of that sequence with proteins of known function (e.g., a viral neuraminidase inhibitor such as Tamiflu), can provide an initial clue as the inhibitors or antagonists of the protein. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Test compounds, such as test neuraminidase inhibitors, are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups. Screening compound libraries listed above [also see EXAMPLES 1 and 2, and U.S. Patent Application Publication No. 2005/0009163, which is hereby incorporated by reference], in combination with biofilm assays described below (such as the one depicted in EXAMPLE 1) can be used to identify neuraminidase inhibitors that disrupt the formation of a biofilm (Lew et al., (2000) Curr. Med. Chem. 7(6):663-72; Werner et al., (2006) Brief Funct. Genomic Proteomic 5(1):32-6).

Small molecule combinatorial libraries can also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318.

In vitro translation-based libraries include but are not limited to those described in PCT Publication No. WO 91/05058; and Mattheakis et al., (1994) Proc. Natl. Acad. Sci. USA 91:9022-9026.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been permethylated to generate a chemically transformed combinatorial library, is described by Ostresh et al. (1994), Proc. Natl. Acad. Sci. USA 91:11138-11142.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1994) Science 263:671-673; and PCT Pub. WO 94/18318. Screening methods of the invention utilizing, for example, the libraries described above, can allow for the identification of candidate neuraminidase inhibitors.

One of skill in the art will be familiar with methods for predicting the effect on protein conformation of a change in protein sequence, and can thus “design” a variant which functions as an antagonist according to known methods. One example of such a method is described by Dahiyat and Mayo in Science (1997) 278:82 87, which describes the design of proteins de novo. The method can be applied to a known protein to vary only a portion of the polypeptide sequence. By applying the computational methods of Dahiyat and Mayo, specific variants of neuraminidase inhibitors confined to regions which bind the active site of a neuraminidase (such as bacterial neuraminidase) can be proposed and tested to determine whether the variant retains a desired conformation. Similarly, Blake (U.S. Pat. No. 5,565,325) teaches the use of known ligand structures to predict and synthesize variants with similar or modified function.

Other methods for preparing or identifying peptides that bind to a particular target are known in the art. Molecular imprinting, for instance, can be used for the de novo construction of macromolecular structures such as peptides that bind to a particular molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Mosbach, (1994) Trends in Biochem. Sci., 19(9); and Wulff, G., in Polymeric Reagents and Catalysts (Ford, W. T., Ed.) ACS Symposium Series No. 308, pp 186-230, American Chemical Society (1986). One method for preparing mimics of neuraminidase inhibitors involves the steps of: (i) polymerization of functional monomers around a known substrate (the template or in this case, the neuraminidase active domain) that exhibits a desired activity; (ii) removal of the template molecule; and then (iii) polymerization of a second class of monomers in, the void left by the template, to provide a new molecule which exhibits one or more desired properties which are similar to that of the template. In addition to preparing peptides in this manner other binding molecules such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are prepared by the free radical polymerization of functional monomers, resulting in a compound with a nonbiodegradable backbone. Other methods for designing such molecules include for example drug design based on structure activity relationships, which require the synthesis and evaluation of a number of compounds and molecular modeling.

A neuraminidase inhibitor according to the method of the invention modulates the activity of a neuraminidase via either reducing the activity of the neuraminidase in the biofilm after the neuraminidase inhibitor is applied, thus inhibiting formation of the biofilm. For example, a reduction in the formation of the biofilm can be measured by looking at a decrease in the surface area covered by the biofilm, thickness, or consistency (such as the integrity of the biofilm).

An inhibition or reduction in a biofilm via treatment with a neuraminidase inhibitor composition (such as a bacterial neuraminidase inhibitor) can be measured via techniques established in the art. These techniques enable one to assess bacterial attachment via measuring the staining of the adherent biomass, to view microbes in vivo via microscopy methods; or to monitor cell death in the biomass in response to toxic agents. The biofilm can be reduced with respect to the surface area covered by the biofilm, thickness, and consistency (for example, the integrity of the biofilm). Non-limiting examples of biofilm assays include microtiter plate biofilm assays, fluorescence-based biofilm assays, static biofilm assays according to Walker et al., ((2005) Infect. Immun. 73(6): 3693-3701), Air-liquid interface assays, colony biofilm assays, and Kadouri Drip-Fed Biofilm assays (Merritt et al., (2005) Current Protocols in Microbiology 1.B.1.1-1.B.1.17). Biofilms (such as their morphology, thickness, and the like) also can be analyzed via confocal microscopy methods (Walker et al., (2005) Infect. Immun. 73(6): 3693-3701). Thus, these biofilm assays (such as the one depicted in EXAMPLE 1) in combination with screening compound libraries as described above can be used to identify neuraminidase inhibitors that disrupt the formation of a biofilm (Lew et al., (2000) Curr. Med. Chem. 7(6):663-72; Werner et al., (2006) Brief Funct. Genomic Proteomic 5(1):32-6).

A reduction in a biofilm indicates that the neuraminidase inhibitor, inhibited formation of the biofilm as determined by observing that the inhibitor modulated the activity or the expression of the neuraminidase protein, because biofilms are comprised of various microorganisms, thus a neuraminidase inhibitor according to the method of the present invention can inhibit such microorganisms from producing a biofilm. Thus, the formation of biofilm by, e.g., of Gram-negative bacteria, Gram-positive bacteria, or a combination thereof, can be inhibited.

Application of a neuraminidase inhibitor to a biofilm can be accomplished by any means such as spraying it onto the biofilm, infusing it into the biofilming, or pipetting into the depth of the biofilm, and the like (e.g., as shown in EXAMPLE 1).

If the neuraminidase inhibitor is to be administered to a subject, it will be in the form of a pharmaceutically acceptable composition or formulation as described below, wherein the composition or formulation is free of toxicity, which satisfies FDA requirements (see Remington: The Science and Practice of Pharmacy, 20^{th} ed., Lippincott Williams & Wilkins, 2000; U.S. Pat. No. 6,030,604). Such a neuraminidase inhibitor composition, comprising compounds or pharmaceutically acceptable salts, can be administered to a subject harboring a biofilm or is at risk of developing a biofilm (for example patient has undergone surgery, implantation, and the like) or is afflicted with a biofilm production-related disorder (discussed below). Administration can occur alone or with other therapeutically effective composition(s) (e.g., antibiotics) either simultaneously or at different times.

Formulations can include those suitable for oral, nasal, topical (including buccal and sublingual), rectal, vaginal and/or parenteral administration. The formulations can conveniently be presented in unit dosage form and can be prepared by any methods well known in the art of pharmacy. The amount of active ingredient which can be combined with a carrier material to produce a single dosage form will vary depending upon the host being treated, the mode of administration. The amount of active ingredient, which can be combined with a carrier material to produce a single dosage form, will generally be that amount of the compound that produces a therapeutic effect. Generally, out of one hundred percent, this amount will range from about 1 percent to about ninety-nine percent of active ingredient, for example, from about 5% to about 70%, or from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention can also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions can also comprise buffering agents. Solid compositions of a similar type can also be employed as fillers in soft and hard-filled gelatin capsules using such excipients as lactose or milk sugars, as well as high molecular weight polyethylene glycols and the like.

A tablet can be made by compression or molding, optionally with one or more accessory ingredients. Compressed tablets can be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets can be made by molding in a suitable machine a mixture of the powdered compound moistened with an inert liquid diluent.

The tablets, and other solid dosage forms of the pharmaceutical compositions of the present invention, such as dragees, capsules, pills and granules, can optionally be scored or prepared with coatings and shells, such as enteric coatings and other coatings well known in the pharmaceutical-formulating art. They can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, or by incorporating sterilizing agents in the form of sterile solid compositions which can be dissolved in sterile water, or some other sterile injectable medium immediately before use. These compositions can also optionally contain opacifying agents and can be of a composition that they release the active ingredient(s) only, or, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms can contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (for example, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The neuraminidase inhibitor composition can optionally comprise a suitable amount of a physiologically acceptable excipient. Non-limiting examples of physiologically acceptable excipients can be liquids, such as water and oils, including those of petroleum, animal, vegetable, or synthetic origin, such as peanut oil, soybean oil, mineral oil, sesame oil and the like; saline; gum acacia; gelatin; starch paste; talc; keratin; colloidal silica; urea and the like. In addition, auxiliary, stabilizing, thickening, lubricating, and coloring agents can be used. For example, the neuraminidase inhibitor composition and physiologically acceptable excipient are sterile when administered to a subject (such as an animal; for example a human). The physiologically acceptable excipient should be stable under the conditions of manufacture and storage and should be preserved against the contaminating action of microorganisms.

Water is a useful excipient when the compound or a pharmaceutically acceptable salt of the compound is administered intravenously. Saline solutions and aqueous dextrose and glycerol solutions can also be employed as liquid excipients, e.g., for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The neuraminidase inhibitor composition can be administered to the subject by any effective route, for example, orally, by infusion or bolus injection, by absorption through epithelial or mucocutaneous linings (e.g., oral, rectal, vaginal, and intestinal mucosa, etc.), intradermal, intramuscular, intraperitoneal, intravenous, subcutaneous, infusion, intranasal, epidural, oral, sublingual, intracerebral, intravaginal, transdermal, rectal, by inhalation, or topical, e.g., to the ears, nose, eyes, or skin.

Pulmonary administration can also be employed, e.g., by use of an inhaler or nebulizer, and formulation with an aerosolizing agent, or via perfusion in a fluorocarbon or synthetic pulmonary surfactant. For example, the neuraminidase inhibitor composition can be formulated as a suppository, with traditional binders and excipients such as triglycerides. Various known delivery systems, including encapsulation in liposomes, microparticles, microcapsules, and capsules, can be used. Thus, the neuraminidase inhibitor composition can be delivered in a vesicle, such as a liposome (see, e.g., Langer (1990) Science 249:1527-1533; Treat et al., Liposomes in the Therapy of Infectious Disease and Cancer 317-327 and 353-365 (1989)).

The neuraminidase inhibitor composition also can be delivered in a controlled-release system or sustained-release system (see, e.g., Goodson, in Medical Applications of Controlled Release, vol. 2, pp. 115-138 (1984)). Other controlled or sustained-release systems previously discussed can be used as well (Langer (1990) Science 249:1527-1533). For example, a pump can be used (Langer (1990) Science 249:1527-1533; Sefton (1987) CRC Crit. Ref Biomed. Eng. 14:201; Buchwald et al., (1980) Surgery 88:507; and Saudek et al., (1989) N. Engl. J Med. 321:574); or polymeric materials can be used (see Langer and Wise (1985) Medical Applications of Controlled Release; CRC Press Inc.,U.S.; Smolen and Ball (1984) Controlled Drug Bioavailability, Drug Product Design and Performance; Ranger and Peppas, (1983) J. Macromol. Sci. Rev. Macromol. Chem. 2:61; Levy et al., (1985) Science 228:190; During et al., (1989) Ann. Neural. 25:351; and Howard et al., (1989) J. Neurosurg. 71:105). The controlled- or sustained-release systems can be placed in proximity of a target of the compound or a pharmaceutically acceptable salt of the compound, e.g., the respiratory tract, thus requiring only a fraction of the systemic dose.

Modulation of neuraminidase activity can also result in the reduction or prevention of the formation of a biofilm on semi-solid and solid surfaces. For example, these surfaces can be the surface of implanted and/or inserted devices (a medical device, a catheter, an infusion set of an insulin pump, a stent, a prosthetic graft); a wound dressing; the oral cavity; the alimentary or vaginal tracts; the ears or eyes; a contact lens, in addition to the cases or containers that hold the lenses when not in use; industrial equipment, or plumbing systems.

Additionally, a neuraminidase inhibitor according to the method of the invention can be applied to a surface of a contact lens or an implantable/insertable device and other surgical or medical devices (such as a medical device, a catheter, the infusion set of an insulin pump, a stent, a prosthetic graft, a wound dressing) or a wound site via covering, coating, contacting, associating with, filling, or loading the device with a therapeutic amount of a neuraminidase inhibitor in any known manner including, but not limited to the following: (1) directly affixing to the implant, device, or wound site a therapeutic agent or composition of the neuraminidase inhibitor (for example, by either spraying the implant or device with a polymer/neuraminidase inhibitor film, or by dipping the implant or device into a polymer/neuraminidase inhibitor solution, or by other covalent or noncovalent means); (2) coating the implant, wound site, or device with a substance, (such as a hydrogel) that will in turn absorb the therapeutic neuraminidase inhibitor composition; (3) interweaving a therapeutic neuraminidase inhibitor composition coated thread (or the polymer itself formed into a thread) into the implant or device or wound site; (4) inserting the implant or device into a sleeve or mesh which is comprised of or coated with a therapeutic neuraminidase inhibitor composition; (5) constructing the implant or device itself with a therapeutic neuraminidase inhibitor composition (or with respect to a wound site, constructing the wound dressing with a therapeutic neuraminidase inhibitor composition; or (6) adapting the implant or device or wound dressing to release the therapeutic neuraminidase inhibitor composition. Specific disease conditions (for example, cystic fibrosis, pneumonia, and the like as described below) that are bacteria-based can also benefit from a treatment that modulates the activity of an enzyme involved in biofilm formation (for example, treatment with a neuraminidase inhibitor).

For example, application of a neuraminidase inhibitor onto the surface of implanted and/or inserted devices (as described above) in order to reduce or prevent bacterial biofilm formation thus allows for long-term implantation and can diminish the resultant likelihood of premature failure of the device due to encrustation and occlusion by such biofilm. The amount of the neuraminidase inhibitor present in a coating, spray, film, and the like (as described above) applied to the surfaces in order to prevent the formation of a bacterial biofilm is an amount effective to inhibit the attachment of microbes onto the surface and/or the synthesis and/or accumulation of biofilm by attached microbes on such a surface.

Methods of the invention can further protect a subject from premature failure of an insertable or implantable device due to encrustation and occlusion by a bacterial biofilm. According to this method, the subject is administered a therapeutically effective amount of the neuraminidase inhibitor of the invention prior to, at the same time, or after an insertable or implantable device is introduced. An effective amount of a neuraminidase inhibitor can refer to the amount of a therapy which is sufficient to reduce or ameliorate the severity and/or duration of a disorder or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent).

The subject is administered the neuraminidase inhibitor that prevents formation of a bacterial biofilm prior to, at the same time, or after the introduction of the implantable/insertable device. Treatment before or after implantation can take place immediately before or after the implantation or several hours before or after implantation, or at a time or times that the skilled physician deems appropriate. According to the present invention, a subject containing a wound site in addition to those subjects receiving implants can harbor a biofilm. For example, a neuraminidase inhibitor can be administered to the subject prior to, during, or after implantation/insertion of a medical device, catheter, stent, prosthesis, and the like or application of a wound dressing. The neuraminidase inhibitor can be administered to the subject according to routes previously described and can further aid in inhibiting biofilm formation on a surface an/or within a subject.

In the case of the oral cavity, the alimentary or vaginal tracts, the ears or eyes, or a contact lens, a therapeutic amount of a neuraminidase inhibitor can be applied via coating, contacting, associating with, filling, or loading the region with a formulation comprising a paste, gel, liquid, powder, tablet, and the like. With respect to the cases or containers that hold the lenses when not in use, industrial equipment, or plumbing systems, an effective amount of a neuraminidase inhibitor can be applied in the same manners as described above. These applications would thus aid in the inhibition of biofilm formation on such surfaces.

In a subject, a biofilm can form on an oral surface (such as teeth, tongue, back of throat, and the like). These biofilms can be associated with day-to-day bacterial activity of natural flora located in such environments, but can also be associated with oral-related disease(s), such as periodontal disease (for example, gingivitis or periodontitis) or dental carries. Application of the neuraminidase inhibitor (according to methods previously described) onto such oral surfaces can inhibit or prevent bacterial biofilm formation. The amount of the neuraminidase inhibitor that can be applied to the surfaces in order to prevent the formation of a bacterial biofilm is an amount effective to inhibit the attachment of microbes onto the surface and/or the synthesis and/or accumulation of biofilm by attached microbes on such a surface.

The neuraminidase inhibitor for use on oral surfaces can comprise a paste formulation (such as toothpaste), which can then be directly applied to the biofilm of such a surface in a subject. The paste formulation can further comprise an abrasive. The neuraminidase inhibitor can also exist as a gel formulation or in liquid formulation. For example, the neuraminidase inhibitor in a liquid formulation (such as a mouthwash) can directly come in contact with the biofilm on the oral surface of a subject.

Other aspects of the invention are directed at methods of treating biofilm production-related disorders in subjects in need thereof. The method entails administering to the subject an effective amount of a neuraminidase inhibitor that reduces biofilm formation in the subject, and then measuring a reduction or inhibition in the growth of biofilm production-related bacteria in the subject. The reduction in bacterial growth is indicative of the reduction in, or inhibition of, biofilm production in the subject, thereby treating the biofilm production-related disorder. For example, the administered neuraminidase inhibitor can reduce the activity of the neuraminidase or alter the expression of the neuraminidase, thereby inhibiting or preventing the formation of a bacterial biofilm.

According to the present invention, modulation of the neuraminidase enzyme (for example, via reducing enzymatic activity or protein expression as described above) can inhibit or reduce biofilm formation due to diminished adherence of microorganisms to a surface or to increased microorganism death. This therapeutic approach thus can be useful for the treatment of biofilm-production-related disorders/conditions and medical-device related infections associated with the formation of microbial biofilms.

Non-limiting examples of biofilm production-related disorders include chronic otitis media, prostatitis, cystitis, bronchiectasis, bacterial endocarditis, osteomyelitis, dental caries, periodontal disease, infectious kidney stones, acne, Legionnaire's disease, chronic obstructive pulmonary disease (COPD), and infections from implanted/inserted devices. In one specific example, subjects with CF display an accumulation of biofilm in the lungs and digestive tract. In subjects afflicted with COPD, such as emphysema and chronic bronchitis, patients display a characteristic inflammation of the airways wherein airflow through such airways, and subsequently out of the lungs, is chronically obstructed. The methods of treatment according to the invention can also benefit a subject having chronic otitis media. Otitis media refers to an infection or inflammation in the middle ear area. The inflammation begins when infections (for example, those caused by bacterial or viral infections) that cause sore throats, colds, or other respiratory/breathing problems spread to the middle ear. Acute otitis media is the presence of fluid, typically pus, in the middle ear with symptoms of pain, redness of the eardrum, and possible fever. However the biofilm production-related disorder can be further classified as chronic if fluid is present in the middle ear for six or more weeks.

Biofilm production-related disorders can also encompass infections derived from implanted/inserted devices (such as those described previously), medical device-related infections, such as infections from biliary stents, orthopedic implant infections, and catheter-related infections (kidney, vascular, peritoneal). An infection can also originate from sites where the integrity of the skin and/or soft tissue has been compromised. Non-limiting examples include dermatitis, ulcers from peripheral vascular disease, a burn injury, and trauma. For example, a Gram-positive bacterium, such as S. pneumoniae, can cause opportunistic infections in such tissues. The ability of S. pneumoniae to infect burn wound sites, e.g., is enhanced due to the breakdown of the skin, burn-related immune defects, and antibiotic selection.

A subject in need of treatment (for example those previously described, such as an animal or human) can be one afflicted with the infections or disorders described above. As such, the subject is at risk of developing a biofilm on or in a biologically relevant surface, or already has developed such a biofilm. Such a subject at risk can be a candidate for treatment with a neuraminidase inhibitor in order to inhibit the development or onset of a biofilm-production-related disorder/condition or prevent the recurrence, onset, or development of one or more symptoms of a biofilm-production-related disorder/condition.

The subject in need can be administered a neuraminidase inhibitor as described above. It can be administered alone or in combination with a second therapeutic, e.g., such as an antibiotic, in order to prevent or inhibit the formation of bacterial biofilms. An antibiotic can be co-administered with the bacterial neuraminidase inhibitor, either sequentially or simultaneously. Upon contacting the cell, the bacterial neuraminidase inhibitor modulates the activity or the expression of the bacterial neuraminidase wherein the inhibitor reduces the activity or the expression of the bacterial neuraminidase, as described above.

An antibiotic refers to any compound known to one of ordinary skill in the art that will inhibit the growth of, or kill, bacteria. Useful, non-limiting examples of an antibiotic include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Perfloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, NC), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Administration of neuraminidase inhibitors to a subject can serve as a treatment that limits the severity and spread of pathogenic infections, such as bacterial infections. Neuraminidase inhibitors intended for human use must be efficacious and function in inhibiting the formation of biofilms, but must also not be toxic. The skilled physician via clinical trials can determine efficacy and toxicity.

An effective amount of a neuraminidase inhibitor refers to the amount of a therapy sufficient to reduce or ameliorate the severity and/or duration of a disorder, such as a biofilm production-related disorder (for example, pneumonia, meningitis, CF, COPD, otitis media, and others described above). An effective amount of a neuraminidase inhibitor can also be sufficient to reduce the degree and time-span of one or more symptoms associated with a biofilm production-related disorder. Additionally, this amount can prevent the advancement of a biofilm production-related disorder, cause regression of such a disorder, prevent the recurrence, development, onset or progression of one or more symptoms associated with a biofilm production-related disorder. The skilled physician can determine a therapeutic dose of a neuraminidase inhibitor that inhibits biofilm formation and/or reduces the duration of a disorder or symptoms thereof. Methods of administration of a neuraminidase inhibitor composition have been described above.

A neuraminidase inhibitor according to the methods of the invention can reduce biofilms associated with a biofilm production-related disorder with respect to the surface area the biofilm covers, thickness, and/or consistency (for example, the integrity of the biofilm). This reduction can be assessed via measuring the growth of bacteria associated with biofilm-production-related disorders, conditions, or diseases. For example, the growth of bacteria of a biofilm-production-related disease can be quantified via measuring the density of bacteria of a biofilm-production-related-disease in a biological sample. Non-limiting examples of biological samples include blood, serum, sputum, lacrimal secretions, semen, urine, vaginal secretions, and tissue samples. The reduction in the growth of bacteria of a biofilm-production-related disease can also be measured by chest x-rays or by a pulmonary function test (PFT) (for example, spirometry or forced expiratory volume (FEV₁)).

In another non-limiting example, the presence or growth of biofilm production-related bacteria can be measured by detecting the presence of antigens of biofilm production-related bacteria in a biological sample, such as those described above. For example, an antibody to S. pneumoniae components can be used as a test for colonization/infection in a subject afflicted with a biofilm production-related condition or disorder, wherein the presence of Streptococcus antigens is detected in a biological sample, such as blood. These antibodies can be generated according to methods well established in the art or can be obtained commercially (for example, from Abcam, Cambridge, Mass.; Cell Sciences Canton, M A; Novus Biologicals, Littleton, Colo.; or GeneTex, San Antonio, Tex.).

Spirometry measures lung function, for example, the volume and/or flow of air that can be inhaled and exhaled. The FEV₁ is a measurement of the volume exhaled during the first second of a forced expiratory maneuver started from the level of total lung capacity. FEV₁ is the most frequently used index for evaluating bronchoconstriction, airway obstruction, or bronchodilatation. These methods are important for assessing biofilm production-related conditions, such as pneumonia, cystic fibrosis, and COPD. A reduction in the growth of bacteria associated with biofilm production-related disorders and/or conditions is indicative of a reduction in or inhibiton of biofilm production.

Methods of the invention are provided that can prevent or reduce biofilm formation (such as a bacterial biofilm) on a biologically relevant surface, wherein a neuraminidase inhibitor is administered to a subject (such as a mammal, for example a human) in order to prevent or reduce the formation of bacterial biofilms. These surfaces include, but are not limited to, an epithelial or mucosal surface of the respiratory tract, lungs, the oral cavity, the alimentary and vaginal tracts, in the ear or the surface of the eye, and the urinary tract. For example, a biofilm can affect the surface of a lung (such as the lung of a subject with pneumonia, CF, or COPD), which is comprised of epithelial cells.

Epithelial cells are named on the basis of their cell type: simple squamous, simple cuboidal, simple columnar, stratified squamous, stratified cuboidal, or stratified columnar epithelia. Such epithelial cells can be obtained from any tissue organ having such cells, for example from the lining of cavities such as the mouth, blood vessels, heart and lungs; from the outer layers of the skin; from the lining of the air passages, stomach, and intestines; in the nose, ears and the taste buds of the tongue; from the lining of the vaginal and urinary tracts, rectum, uterus, and oviducts, and from the larger ducts of certain glands and the papillary ducts of the kidneys. Epithelial cells can also be obtained from in vitro epithelial cell culture systems well known in the art (see, e.g., Harris, A. (ed.), (1996) Epithelial Cell Culture, Cambridge University Press). Such cell lines can be available commercially or can be generated via standard cell culturing techniques (see e.g. Harris, supra).

Other aspects of the current invention are directed to methods that are useful for treating a subject (such as an animal or human) that has, is developing, or is at risk of developing a biofilm-production-related disorder/condition. A subject who is developing a biofilm-production-related disorder/condition is an individual harboring an immature biofilm clinically evident or detectable to the skilled artisan, but that has not yet fully formed. A subject at risk of developing a biofilm can be one in which the introduction of a medical device, a graft implantation, and the like is scheduled. The risk of developing a biofilm can also be due to a biofilm production-related disease (such as the channel transporter mutation associated with CF) that is in its earlier stages, e.g., no bacterial infection and/or biofilm formation is yet detected.

In a specific example, methods are provided for preventing biofilm formation in the airways of cystic fibrosis patients who are free of bacterial infection of the airways. Such patients are at risk of developing a biofilm, and as such, are “in need thereof” The method entails administering to the subject an effective amount of a neuraminidase inhibitor, which prevents growth of bacteria associated with a biofilm production-related disorder in the airways of a subject, and detecting the absence of such bacterial growth in the airways of the subject. The absence of bacterial growth is indicative of the lack of biofilm formation in the airways of the subject. For example, the subject can be one afflicted with CF and is a human (such as an individual of 5 years of age or less) that has not yet developed a bacterial infection of the airways indicating that P. aeruginosa and/or S. pneumoniae has not yet colonized the epithelial cells of the lung airways. Airways of the lung include bronchii, bronchioles, aleveolar ducts, alveolar sacs, and alveoili.

The growth of bacteria associated with a biofilm-production-related disorder can be quantified by detecting the presence of S. pneumoniae (e.g. by measuring the density of the bacteria) in a biological sample according to methods practiced in the art. Non-limiting examples of biological samples include blood, serum, sputum, lacrimal secretions, sweat, semen, urine, vaginal secretions, and tissue samples. For example, the presence or absence of bacteria can be measured via detecting the presence of bacterial in a biological sample, such as those described above. An antibody to S. pneumoniae components can be used as a test for colonization/infection in a subject afflicted with a biofilm production-related condition or disorder (such as pneumonia or CF), wherein the presence of Streptococcus antigens is detected in a biological sample, such as blood. These antibodies can be generated according to methods well established in the art or can be obtained commercially (for example, from Abcam, Cambridge, Mass.; Cell Sciences Canton, M A; Novus Biologicals, Littleton, Colo.; or GeneTex, San Antonio, Tex.). The absence of bacterial growth and its associated biofilm can also be measured, e.g., by chest x-rays or by a pulmonary function test (PFT) (for example, spirometry or FEV₁, methods described above).

According to the invention, administration of neuraminidase inhibitors to a subject (for example, one afflicted with CF who is free of bacterial infection in the airways) can serve as a preventive means by which to deter the development of pathogenic infections, such as bacterial infections (eg. P. aeruginosa and/or S. pneumoniae).

An effective amount of a neuraminidase inhibitor to be administered can be the amount sufficient to prevent the onset or development of a pathogenic infection associated with a biofilm production-related disease or disorder (for example, pneumonia, COPD, or CF). The skilled physician can determine a therapeutic dose of a neuraminidase inhibitor that prevents pathogenic infection in addition to biofilm formation. An effective amount of a neuraminidase inhibitor, for example, one directed at the Streptococcus enzyme, can be administered according to methods of this invention. Methods of administration of a neuraminidase inhibitor composition have been described above.

Aspects of the present invention also provide methods of preventing or reducing biofilm formation associated with a wide variety of commercial, industrial, and processing operations, such as those found in water handling/processing industries. The method for inhibiting biofilm formation on an industrial/commercial surface entails applying a neuraminidase inhibitor to the biofilm found on such surfaces. The neuraminidase inhibitor modulated activity or expression of the neuraminidase protein can then be measured. A reduction in the neuraminidase inhibitor modulated activity or expression of the neuraminidase protein is indicative of the inhibition of biofilm formation. The neuraminidase inhibitor can be directed at any neuraminidase produced by organisms in the biofilm. These have been described above.

The neuraminidase inhibitors useful in the invention that prevent or reduce the formation of bacterial biofilms can be utilized in order to prevent microorganisms from adhering to surfaces. These surfaces can be hard, semi-hard, porous, soft, semi-soft, regenerating, or non-regenerating; and can include, but are not limited to, metal, alloy, polyurethane, water, polymeric surfaces of implantable/insertable devices (such as medical devices or catheters), the enamel of teeth, and surfaces of mammalian cellular membranes.

For example, some surfaces can be the surfaces of industrial equipment (such as, equipment located in Good Manufacturing Practice (GMP) facilities, food processing plants, photo processing venues, and the like), the surfaces of plumbing systems, or the surfaces bodies of water (such as lakes, swimming pools, oceans, and the like). Embodiments of the invention further provide methods for inhibiting and/or reducing biofilm formation within a plumbing system.

The surfaces can be coated, sprayed, or impregnated with a neuraminidase inhibitor prior to use to prevent the formation of bacterial biofilms. Surfaces also can be treated with a neuraminidase inhibitor to reduce, control, or eradicate microorganisms (such as those described above) adhering to such surfaces. In a specific example, the method can be used in an open re-circulating water system used for cooling to control the temperature of fermentation tanks. In such a system, the water circulates through coils and jackets in the tank, over an induced draft-cooling tower, and then is pumped back from the sump. Biofilm-producing microorganisms can flourish in the cooling water system due to contamination and highly nutritive substances from the surrounding environment (Coetser et al., (2005) Crit. Rev. Micro. 31: 212-32). This biofilm can form on the cooling tower water distribution elements, its support components, and on the heat transfer surfaces of the system resulting in poor cooling efficiency. Thus, to prevent formation of the biofilm, a neuraminidase inhibitor is applied to treat the water-cooling system. Not only is the treatment suitable for the water-cooling system of a fermentation tank, but can also be applicable to air conditioning condensers, (such as those found in hospitals or industrial plants), that are served by a rooftop open-deck cooling tower (described in U.S. Pat. No. 6,395,189 and U.S. Appln. Pub. No. 2005/0158253).

The neuraminidase inhibitor can be added directly to a water handling or collection system (such as the systems described above). Alternatively, the bacterial neuraminidase inhibitor can be applied to the biofilm, itself, or to the bacteria within, or the producers of the biofilm or which can produce the biofilm. It can be applied as a formulation comprising a paste, liquid, powder, gel, or tablet. The neuraminidase inhibitor functions via modulating the activity or the expression of a bacterial neuraminidase protein. Upon the neuraminidase inhibitor contacting the bacterial cell, the activity or expression of the bacterial neuraminidase is reduced, thereby preventing or reducing the formation of a bacterial biofilm. For example, the biofilm formed on the surfaces of systems (which include but are not limited to plumbing, tubing, and support components) involved with water condensate collections, sewerage discharges, paper pulping operations, re-circulating water systems (such as air conditioning systems, a cooling tower, and the like), and, in water bearing, handling, processing, collection systems of an industrial setting can be formed by a Gram-negative or Gram-positive bacterium (as described above), or a combination thereof.

Adding the neuraminidase inhibitor prevents or reduces formation of biofilms on the surface of the water or on the surfaces of the pipes or plumbing of water-handling systems, or other surfaces of the collection and/or operation systems that the water contacts.

Also provided are methods for identifying or screening for inhibitors of a neuraminidase protein useful in preventing or inhibiting the formation of biofilms. The method entails contacting a cell infected with a biofilm-producing microbe, such as a protozoa, yeast, virus, or bacterium, (e.g., Sreptococcus) with a test (or candidate) neuraminidase inhibitor, and then determining whether the test neuraminidase inhibitor inhibits biofilm formation Inhibition of biofilm formation thus is indicative of the ability of the test neuraminidase inhibitor to prevent or inhibit microbial infection.

Inhibition of biofilm formation can be determined by any known method, such as a visual method performed with the aid of a microscope, colorimterically via densitometry, and the like. Neuraminidase inhibitors that reduce or prevent the formation of a biofilm on surfaces are described or can be identified via biofilm assays as described above (see, e.g., EXAMPLE 1). Thus, one skilled in the art can carry out any known biofilm assay, such as those previously described.

Neuraminidase gene products, including polynucleotides, oligonucleotides and polypeptides, can be used in screening assays to identify compounds that specifically bind to bacterial, viral, yeast, or protozoan neuraminidase gene products and thus have potential use as agonists, or antagonists of such neuraminidases. In one embodiment, the bacterial, viral, yeast, or protozoan neuraminidase polynucleotides and polypeptides of the invention are useful to screen for compounds that affect the sialidase or biofilm formation activities of bacterial, viral, yeast, or protozoan neuraminidase gene products.

The invention thus provides assays to detect molecules that specifically bind to bacterial, viral, yeast, or protozoan neuraminidases. For example, recombinant cells expressing a gene encoding bacterial, viral, yeast, or protozoan neuraminidase can be used to recombinantly produce a bacterial, viral, yeast, or protozoan neuraminidases polypeptide, respectively, and to screen for molecules that bind to a bacterial, viral, yeast, or protozoan neuraminidases polypeptide, respectively. Methods that can be used to carry out the foregoing are commonly known in the art.

A neuraminidase inhibitor that can be used according to the invention has been described above.

Non-limiting examples of cells to be contacted with the neuraminidase inhibitor include bacterial cells, yeast cells, protozoan cells, and cells infected with a viral or other pathogen. Representative bacteria include but are not limited to Legionella sp., P. aeruginosa, H. influenzae, V. cholerae, Yersinia pestis, Escherichia coli, and Streptococcus pneumoniae. Alternatively, the cell to be contacted is an animal cell, such as a mammalian cell, or more specifically, a human cell. The cell can be from a particular tissue or cell line, such as an epithelial cell.

Another aspect of the invention is directed to a mutant S. pneumoniae strain having a deletion in the gene encoding a neuraminidase protein. Deleting a portion of the gene so that the gene cannot function can be accomplished by mutation or insertion of another DNA in the base sequence of the gene (also referred to as a gene disruption). As a result, the gene cannot be transcribed into mRNA, the structural gene is not translated, and the transcription product mRNA becomes incomplete. A mutation or deletion occurs in the amino acid sequence of the translation product or structural protein, rendering the protein unable to perform its original function.

Any method known in the art can be used for constructing a gene-disrupted strain, such as a strain wherein the gene encodes a neuraminidase protein. For example, the gene disruption can occur via homologous recombination or other methods described in Nickoloff (ed.), (1995) Methods in Molecular Biology 47: 291-302, Humana Press Inc., Totowa, N.J.; or in Sambrook et al. (eds.), Molecular Cloning: A Laboratory Manual, Second Edition (1989) Cold Spring Harbor Laboratory Press.

### EXAMPLES

Examples are provided below to facilitate a more complete understanding of the present invention. The following examples illustrate the exemplary modes of making and practicing the present invention. However, the scope of the invention is not limited to specific embodiments disclosed in these Examples, which are for purposes of illustration only, since alternative methods can be utilized to obtain similar results.

### Example 1

### Structural Analysis and Inhibitor Identification of Respiratory Pathogen Neuraminidases

There is a need to develop therapeutic agents for the treatment of infections by the human respiratory pathogens Pseudomonas aeruginosa and Streptococcus pneumoniae. P. aeruginosa causes significant morbidity and mortality for cystic fibrosis patients and is a leading nosocomial pathogen. S. pneumoniae is the most common cause of bacterial pneumonia and otitis media leading to substantial economic burden. The neuraminidases of P. aeruginosa and S. pneumoniae, which catalyze the release of terminal sialic acid residues from glycoconjugates, are involved in host colonization in animal models of infection. We demonstrate that the S. pneumoniae neuraminidase (NanA), like that of P. aeruginosa (NanPs), contributes to biofilm formation. To help identify compounds that inhibit these neuraminidases, the crystal structures of the P. aeruginosa and S. pneumoniae enzymes were soved at 1.6 and 1.7 Å resolution, respectively. The active site regions of the two enzymes are strikingly different: NanA contains a deep pocket that is similar to that in canonical neuraminidases, while the NanPs active site is much more open. The structural information was used to undertake a ligand-receptor docking screen and a lead compound that shows inhibition against both enzymes was identified. This work can be the basis for developing drugs to prevent colonization of the respiratory tract by these two important pathogens.

P. aeruginosa and S. pneumoniae are important human respiratory pathogens. The neuraminidase enzyme, which cleaves sialic acid residues, plays an important role in the pathogenesis of respiratory infection. We demonstrate that the NanA neuraminidase of S. pneumoniae like the NanPs enzyme of P. aeruginosa is involved in biofilm formation, which enhances airway colonization. We determined the three-dimensional structures of the neuraminidase from these two organisms and show that although they possess similar overall structure, their active sites are vastly different. Using this structural information we identified candidate inhibitory compounds. The information derived from the structures of the two enzymes and the subsequent identification of inhibitory compounds represents a first step in the development of therapeutics against these two significant human pathogens.

The production of neuraminidase/sialidase by mucosal pathogens has long been associated with the pathogens of respiratory tract infection [A1]. Neuraminidases are widespread among animals and microorganisms, catalyzing the release of terminal sialic acid residues from glycoconjugates [A2]. The influenza neuraminidase is required to facilitate spread of the virus, cleaving it from its sialic acid receptor. The influenza neuraminidase is not only a key antigen for the highly successful influenza vaccine, it is also the target for the drugs zanamivir and oseltamivir that are widely used to prevent and ameliorate influenza infection [A3]. The analysis of the structure/function relationships of the influenza neuraminidase and its substrate provides a paradigm for the development of antimicrobial therapy directed at critical virulence factors. These neuraminidases are recognized in most sequence analysis programs by their common elements, often conserved “ASP” boxes, sites in the predicted amino acid sequence that are predicted to interact with sialic acid residues [A2,A4]. Neuraminidases are produced by respiratory pathogens with very different metabolic requirements and highly variable potential for virulence. As neuraminidases are highly conserved virulence factors that appear to be critical for colonization and infection of the respiratory tract by such diverse pathogens, the development of anti-bacterial neuraminidase inhibitors would appear to be a realistic therapeutic target.

S. pneumoniae is the most common cause of bacterial pneumonia and a significant cause of otitis media, a major clinical problem in pediatrics [A5,A6]. There are at least 90 serotypes of S. pneumoniae, with the current pediatric vaccine covering seven common and the adult vaccine covering 23 serotypes [A5,A7]. There is a need for therapeutic strategies active against all serotypes as those serotypes not covered by the vaccines are rising in prevalence by genetic recombination and are increasingly associated with invasive disease [A8, A9]. In addition, antibiotic resistance amongst S. pneumoniae is a growing problem. S. pneumoniae produces at least three distinct neuraminidases [A10]; NanA being the most expressed and active [A11, A12] that is conserved in all strains [A10, A13, A14]. Production of NanA can be detected in vivo, and its expression is upregulated upon interaction with host cells [A15-A18]. The pneumococcal neuraminidase modifies host glycoconjugates [A19, A20] and exposes potential binding receptors [A21-A25]. Without being bound by theory, a role in survival has also been indicated as desialylation of other organisms has been demonstrated [A26]. Pneumococcal neuraminidase activity also provides a source of carbohydrates for bacterial metabolism, cleaving sugars from the mucosal surface [A20, A27, A28], but whether this significantly contributes to bacterial growth in vivo is not clearly established. Several studies have stated that nanA mutants colonize the rodent respiratory tract less efficiently than wild type strains [A11, A29, A30] and vaccination with purified NanA affords some protection against nasopharyngeal colonization and otitis media [A31-A33]. However, these differences can be mouse strain and animal model dependent [A19, A20, A34, A35].

P. aeruginosa, also a neuraminidase producer, is an opportunistic pathogen which is the most common cause of respiratory tract infection in patients with cystic fibrosis and rarely a cause of infection in otherwise healthy subjects [A36]. The neuraminidase of P. aeruginosa (NanPs) has been characterized and found to be important in the pathogenesis of pneumonia in a mouse model of infection [A37]. Its expression is correlative with initial airway colonization, particularly in isolates from young patients recently infected with the organism [A38]. In contrast to the pneumococcal enzyme, the P. aeruginosa neuraminidase does not appear to target host glycoconjugates but instead is involved in the biosynthesis of the extracellular polysaccharides on the bacterial surface that are involved in cell-cell interaction, agglutination and biofilm formation [A37]. P. aeruginosa does not ferment the sugars that are released through neuraminidase activity and expression of the enzyme does not enhance bacterial growth rates [A37]. Nonetheless, the P. aeruginosa neuraminidase mutant is significantly less proficient in colonizing and infecting the lungs of mice and can provide a target for prevention of P. aeruginosa infection in susceptible patients.

Neuraminidase expression by both S. pneumoniae and P. aeruginosa facilitates their respective abilities to colonize and infect respiratory tract in animal models. We show that S. pneumoniae, like P. aeruginosa, uses the neuraminidase in biofilm formation. Without being bound by theory, differences in structures of these enzymes can explain their functional differences and their distinctive roles in pathogenesis. We report here the crystal structures of P. aeruginosa and S. pneumoniae neuraminidases at the 1.6 and 1.7 Å level and using this information have identified a number of compounds that were shown to inhibit the enzymes.

Results

Structure of the NanA neuraminidase from S. pneumoniae. The crystal structure of the free enzyme of S. pneumoniae NanA has been determined at 1.7 Å resolution (Table 1). The bacterial expression construct contained residues 116-1035 and in situ proteolysis with trypsin was essential for the crystallization. The current atomic model contains residues 320-793 and 317-793 for the two NanA molecules in the crystallographic asymmetric unit, respectively. Roughly 200 residues from both the N and C termini of the recombinant protein were removed by trypsin during crystallization. The two NanA molecules have essentially the same conformation, with an rms distance of 0.25 Å for their equivalent Cα atoms. Purified NanA is monomeric in solution, based on gel-filtration chromatography.

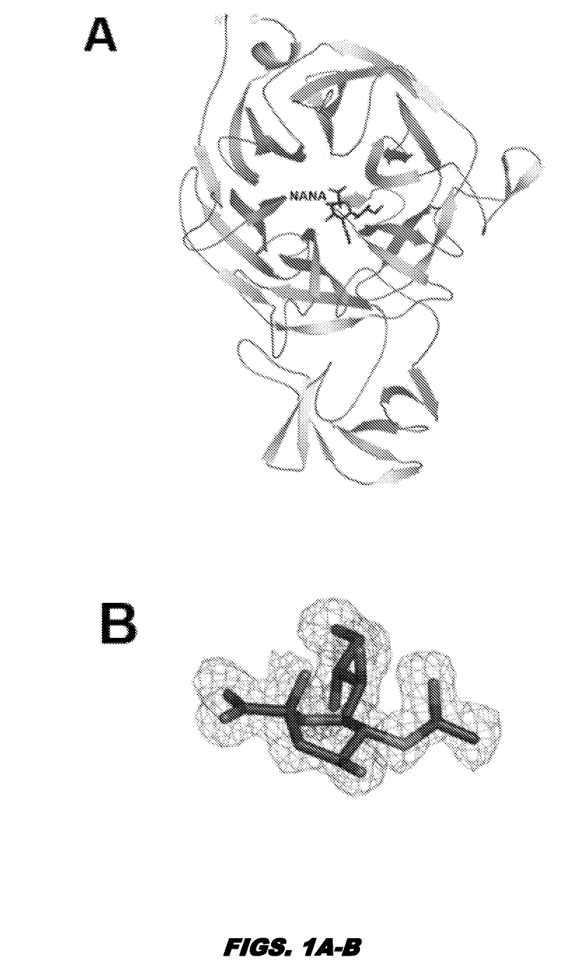

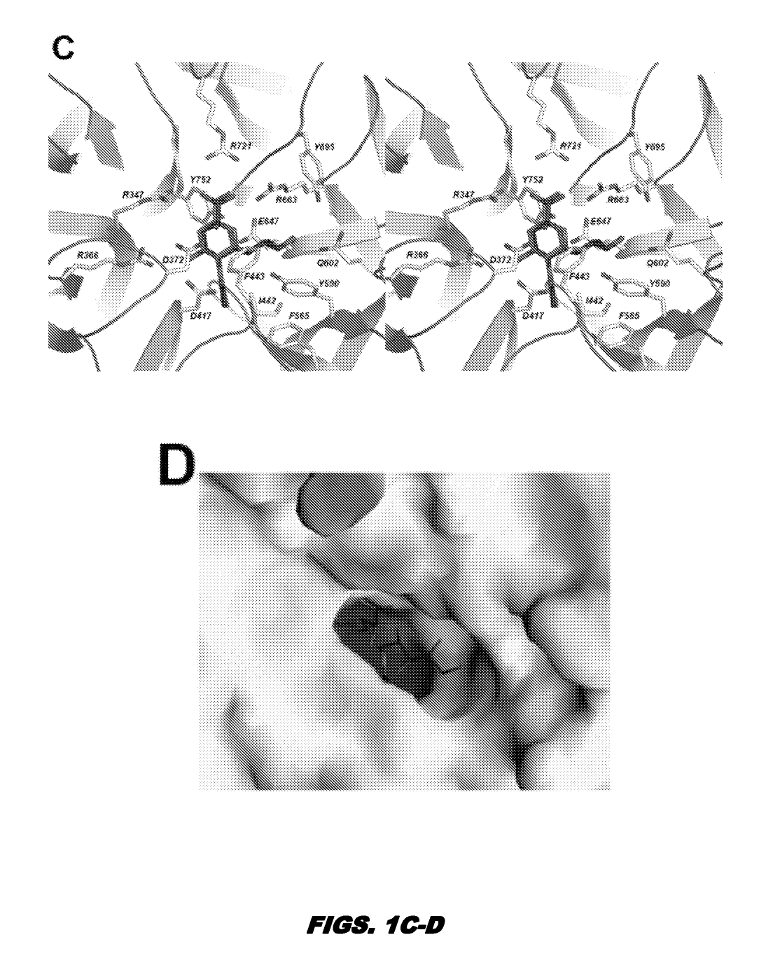

The NanA structure contains a six-bladed β-propeller domain, with an insertion (residues 437-535) between the second and the third β-strands of the second blade (FIG. 1A). This insertion forms a distinct domain located next to the catalytic β-propeller domain. The overall structure of NanA shares high structural similarity with other bacterial neur-aminidases, including the Salmonella typhimurium LT2 neuraminidase [A39], Vibrio cholerae neuraminidase [A40], Clostridium perfringens sialidase NanI [A41], and Micro-monospora viridifaciens sialidase [A42]. The structures of S. pneumoniae NanA in complex with the transition state analog 2,3-dehydro-2-deoxy-N-acetylneuraminic acid (DANA) at 2.5 Å resolution [A43] and NanB [A44] were recently reported. The published NanA structure [A43] is based on an expression construct that contains residues 319-822, although only residues 322-791 are observed in the structure and the crystals are in a different space group. Nonetheless, the overall structure and the interactions with DANA are similar to observations based on our structures. The overall rms distance in equivalent Cα positions is 0.4 Å.

To reveal the molecular mechanism for substrate/inhibitor recognition, we have determined the structures of NanA in complex with the sialic acid compound N-acetylneuraminic acid (NANA) and the transition state analog DANA at 1.7 Å resolution (Table 1). There is essentially no conformational change in the protein upon the binding of these inhibitors. The rms distance in equivalent Cα atoms among the structures of the free enzyme, the NANA complex, and the DANA complex is about 0.15 Å. In addition, there is essentially no conformational change in the active site of the enzyme, where the inhibitors bind.

TABLE 1
Summary of crystallographic information

| | | | NanA in | NanA in |
|---|---|---|---|---|
| | | | complex | complex |
| | NanPs free | NanA free | with | with |
| | enzyme | enzyme | DANA | NANA |
| Resolution | 30-1.6 | 30-1.7 | 30-1.7 | 30-1.7 |
| range (Å) | | | | |
| Number of | 377,554 | 636,912 | 279,534 | 251,113 |
| observations | | | | |
| R_{merge} (%)^{a} | 8.8 (36.5) | 4.1 (9.4) | 3.0 (7.3) | 6.0 (14.2) |
| I/σI | 26.2 (4.9) | 38.8 (10.7) | 31.1 (11.5) | 25.3 (6.6) |
| Redundancy | 4.3 (3.9) | 3.4 (3.2) | 3.0 (2.5) | 2.8 (2.5) |
| Number of | 85,363 | 183,283 | 93,563 | 86,772 |
| reflections | | | | |
| Completeness | 96 (91) | 92 (90) | 93 (86) | 87 (70) |
| (%) | | | | |
| R factor (%) | 17.2 (21.7) | 17.2 (20.4) | 17.8 (20.5) | 17.5 (20.8) |
| Free R factor | 19.1 (23.7) | 19.8 (23.4) | 20.9 (24.7) | 20.9 (24.8) |
| (%) | | | | |
| Residues in | 86 | 86 | 86 | 86 |
| most favored | | | | |
| region of the | | | | |
| Ramachandran | | | | |
| plot (%) | | | | |
| rmsd in bond | 0.005 | 0.005 | 0.007 | 0.005 |
| lengths (Å) | | | | |
| rmsd in bond | 1.4 | 1.4 | 1.3 | 1.4 |
| angles (°) | | | | |
| Protein Data | | | | |
| Bank entry | | | | |
| code | | | | |

^{a}The numbers in parenthesis are for the highest resolution shell.

Clear electron density was observed for the NANA inhibitor, which is bound to NanA in a boat conformation (FIG. 1B). As observed in structures of NANA in complex with other neuraminidases, the compound is involved in a large network of ionic and hydrogen-bonding interactions with the active site of NanA (FIG. 1C; see also Table 3). The carboxyl group of NANA interacts with three Arg residues (Arg347, Arg663 and Arg721), and the amide nitrogen of NANA is hydrogen-bonded to the side chain of Asp417. The two hydroxyl groups on the ring of NANA interact with the side chains of Arg366, Asp417 and Asp372, the last of which is also hydrogen-bonded to one of the hydroxyls on the glycerol group. In addition to the polar interactions, several hydrophobic side chains are located near the inhibitor. Most importantly, residues Ile442 and Phe443 in the long loop at the beginning of the insertion in blade 2 (FIG. 1A) cover up part of the inhibitor and greatly reduce the size of the active site pocket, producing a tight fit between the inhibitor and the enzyme (FIG. 1D). DANA was bound in the same position as NANA, showing the same interactions with the enzyme except for the loss of the hydrogen-bond with Asp372 due to the absence of the hydroxyl group on the ring (FIG. 1C).

TABLE 3
Amino acid residues corresponding to the putative active site/
neuraminidase inhibitor-binding site (residues within 10 Å).

| | | | | | | | |
|---|---|---|---|---|---|---|---|
| Arg347 | Arg366 | Asp372 | Asp417 | Ile442 | Phe443 | Phe565 | Tyr590 |
| Gln602 | Glu647 | Arg663 | Tyr695 | Tyr752 | Arg 721 | | |

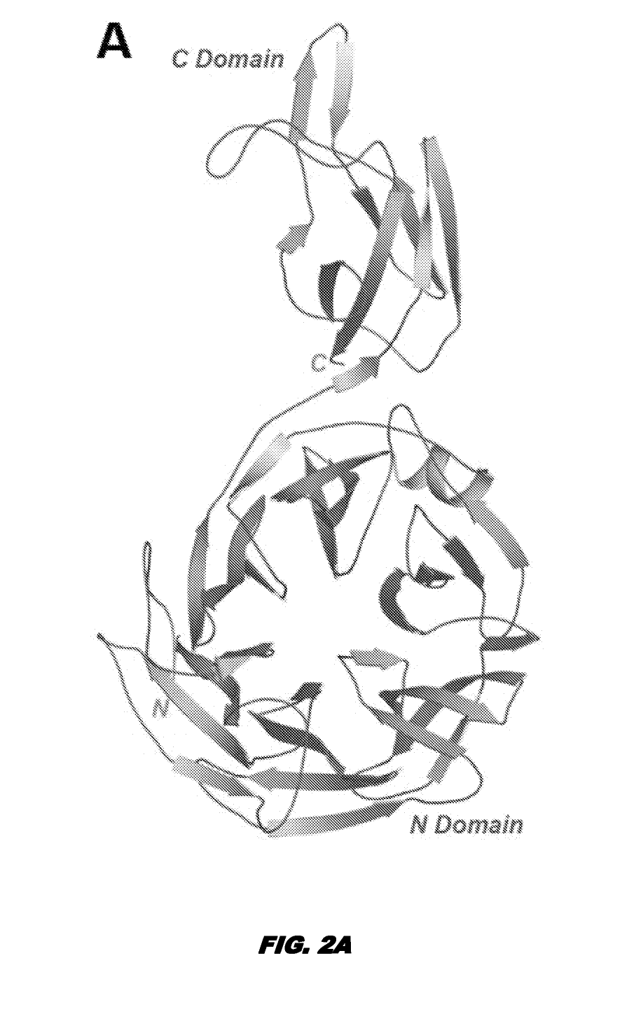

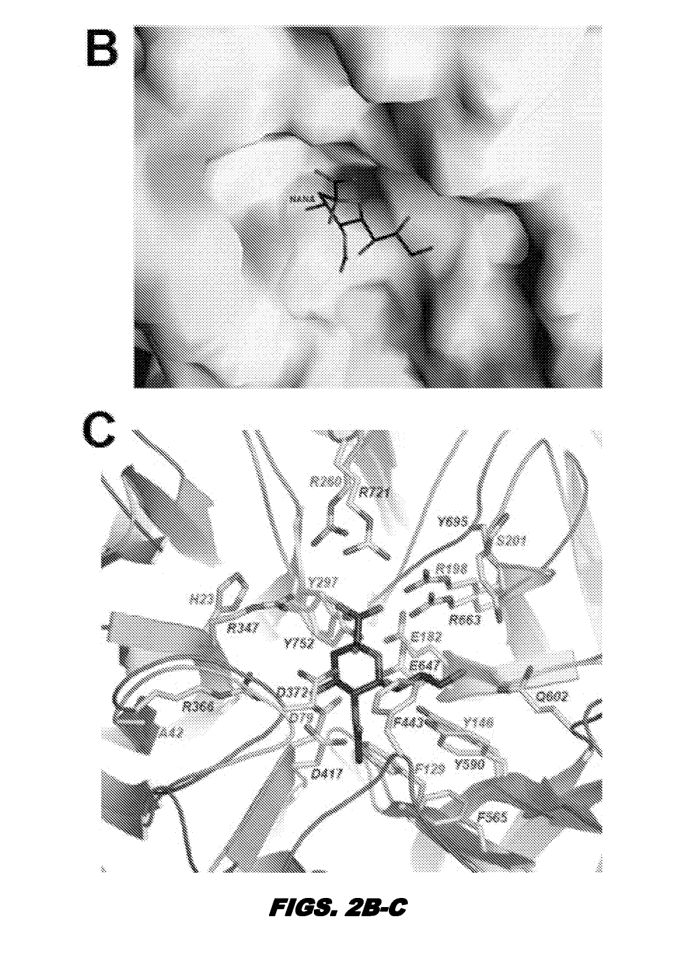

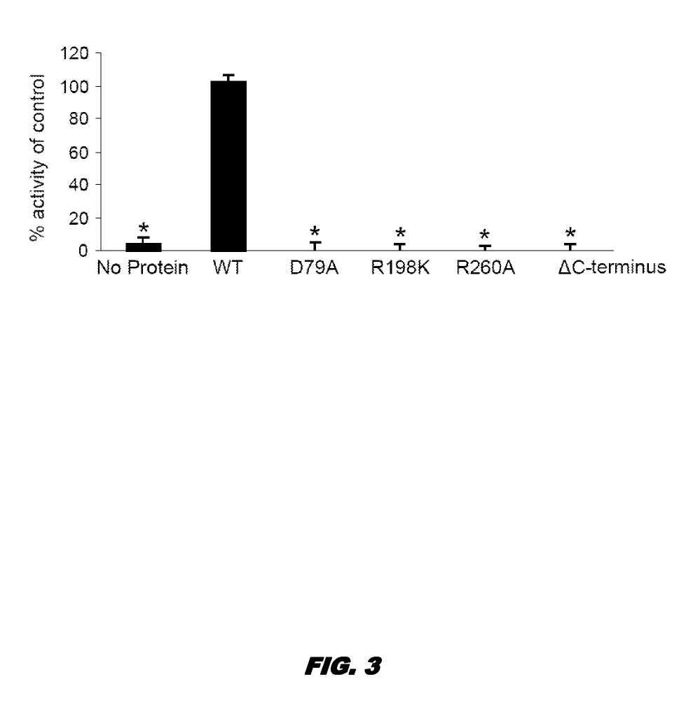

Structure of the P. aeruginosa neuraminidase reveals exposed active site. The structure of the P. aeruginosa neuraminidase (NanPs) at 1.6 Å resolution was also determined (Table 1). The enzyme, previously referred to as NanA [A37], product of PA2794, has been renamed to reflect its differences to typical bacterial neuraminidases. The structure contains a six-bladed β-propeller in the N-terminal region (residues 1-334, FIG. 2A) and a C-terminal domain (C domain, residues 335-438) that belongs to the immuno-globulin superfamily, based on comparisons with other structures in the Protein Data Bank using the SSM server [A45]. A close structural homolog of the C domain is the immuno-globulin superfamily domain of the muscle protein twitchin [A46]. The rms distance among equivalent Cα atoms of the two structures is 3 Å, although the amino acid sequence identity is only 6%. This domain is located about 50 Å away from the active site of the enzyme (FIG. 2A); although our mutagenesis studies showed that it is important for the catalytic activity (FIG. 3). The domain mediates the formation of a trimer in the crystal, although the protein is monomeric in solution, based on gel-filtration and light scattering experiments.

The β-propeller domain of P. aeruginosa NanPs has the same overall structure as that of canonical neuraminidases. The rms distance among equivalent Cα atoms in P. aeruginosa NanPs and these other structures is 2.5-3 Å, but the amino acid sequence identity is 7-19%. Some of the important residues in the active site are also conserved in NanPs, consistent with our observation that it confers some neuraminidase activity [A47]. We constructed Asp79Ala (Asp417 in NanA) and Arg260Ala (Arg721 in NanA) mutations in the active site of NanPs and found these residues to be important for activity (FIG. 3). The mutation of Arg292 in the influenza neuraminidase, equivalent to Arg198 in NanPs and Arg663 in NanA, to a lysine confers resistance to the inhibitor oseltamivir [A48]. However, the Arg198Lys mutant of NanPs showed loss of activity (FIG. 3).

There are dramatic differences in the shape of the active site region between NanPs and the other neuraminidases. Compared to NanA (FIG. 1D), the active site region of NanPs is much more open (FIG. 2B). An important cause of this difference is the lack of the insertion in the second blade of the propeller. The long loop at the beginning of this insertion, especially the side chains of Ile442 and Phe443 (FIG. 1C), is absent in NanPs (FIG. 2C), leading to a much more exposed active site. Additionally, variations at other amino acid residues also contribute to the different active site topography of NanPs. For example, the side chain of Phe129 in NanPs clashes with the bound position of the acetyl group in NANA (FIG. 2C), and this is the only recognizable clash between the inhibitor and the NanPs active site (FIG. 2B). The equivalent residue in NanA is Gly567.

Several other residues in the NANA binding site are not conserved in NanPs (FIG. 2C). Especially, of the four arginine residues that interact with NANA in NanA, two (Arg347 and Arg366) are replaced with other residues (His23 and A1a42) in NanPs, while the other two assume different conformations in NanPs (FIG. 2C). In addition, Asp372 in NanA is replaced by Gly47 in NanPs. The structural information shows that the active site region of NanPs is not a good fit for the NANA inhibitor (FIG. 2B), indicating that NanPs is probably not a conventional neuraminidase, and its natural substrate(s) remain to be identified.

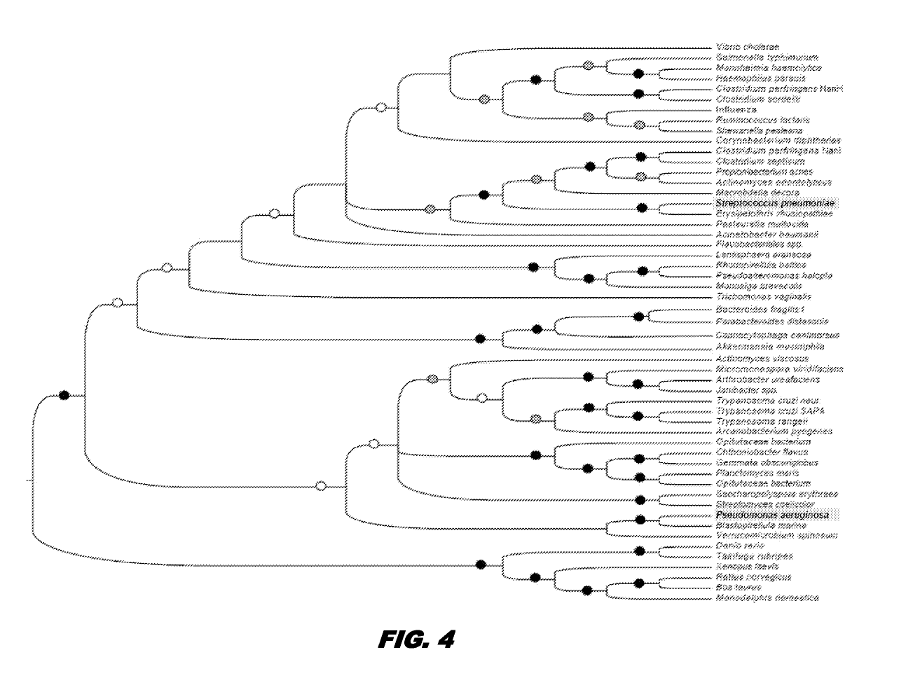

Phylogeny of the S. pneumoniae and P. aeruginosa neuraminidases. To better ascertain the evolutionary relatedness of NanA and NanPs, a phylogenetic analysis of a number bacterial, eukaryotic and viral neuraminidases was conducted (FIG. 4). The neuraminidase superfamily is known to be highly divergent [A2, A4], and, NanA and NanPs cluster at separate branches of the tree. NanA was aligned closest to the erysipeloid-causing bacterium Erysipelothrix rhusiopathiae [A49] (FIG. 4). NanA also clustered closely to the large neuraminidase of C. perfringens [A41] and was more closely related to well characterized bacterial neuraminidases than NanPs. NanPs was part of a deeply rooted branch that was closer to the trypanosome trans-sialidases than the enteric neuraminidases. NanPs was most closely matched to a putative neuraminidase from the aquatic organism Blastopirellula marina [A50].

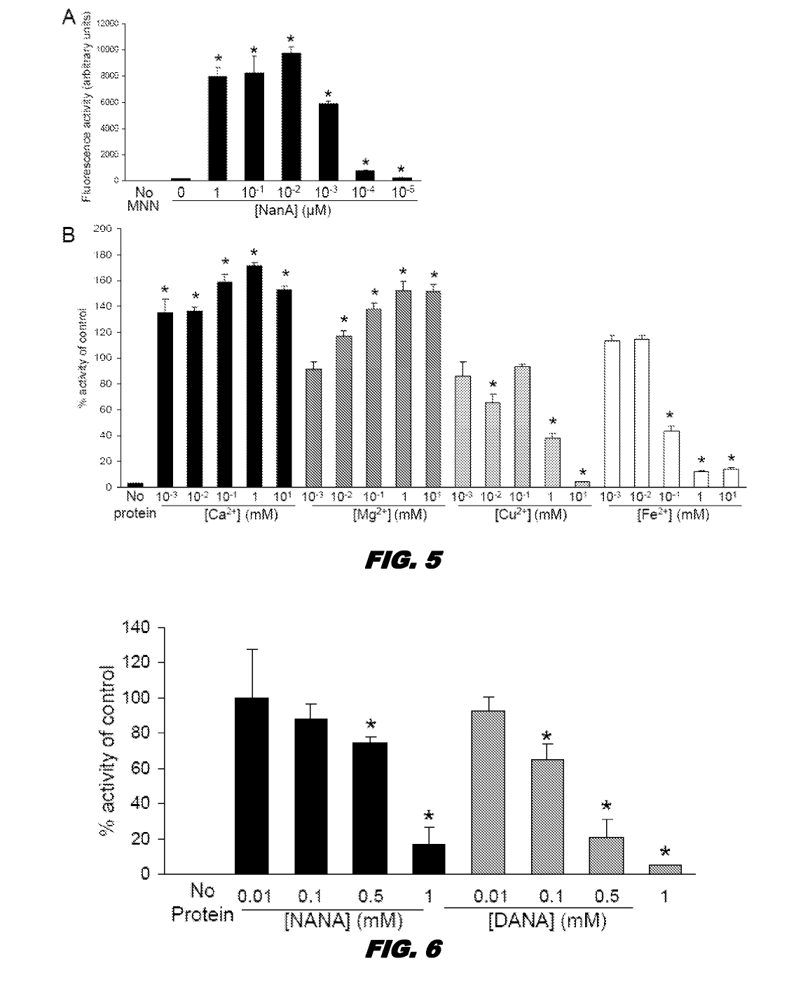

Biochemical properties of pneumococcal NanA. The biochemical activity of NanA was assayed using the fluorogenic sialic acid derivative 2′-(4-methylumbelliferyl)-α-D-N-acetylneuraminic acid (MNN). NanA was observed to cleave the fluorogenic substrate significantly at low nanomolar and even at picomolar concentrations (FIG. 5A). The Km of NanA for this substrate is about 1.4 mM, which is generally comparable to the Km values reported for other neuraminidases. The neuraminidase from Vibrio cholerae requires divalent cations, specifically calcium, to be active [A40, A51]. Although calcium was not essential for NanA activity, an increase in activity of 70% was observed at a Ca²⁺ concentration of 1 mM (FIG. 5B). We observed a 50% activity increase in the presence of magnesium ions (FIG. 5B) and decreased activity in the presence of iron and copper, presumably due to their larger molecular masses (FIG. 5B). The presence of either copper or ferric ions decreased activity by 90% or greater at millimolar levels, indicating that NanA can possess a cation binding site.

We next tested the ability of sialic acids to competitively inhibit NanA activity. NANA was able to cause 50% inhibition at 600 μM (FIG. 6). We observed higher inhibition with the transition state analog DANA compared to NANA. DANA was able to reduce activity by 50% at 200 μM and this inhibitory potency is in the range that is observed for other neuraminidases [A39, A52]. As a comparison, NANA and DANA possess very weak inhibitory activity towards the P. aeruginosa neuraminidase, 50% inhibition being achieved in the 10 millimolar range with both compounds [A37].

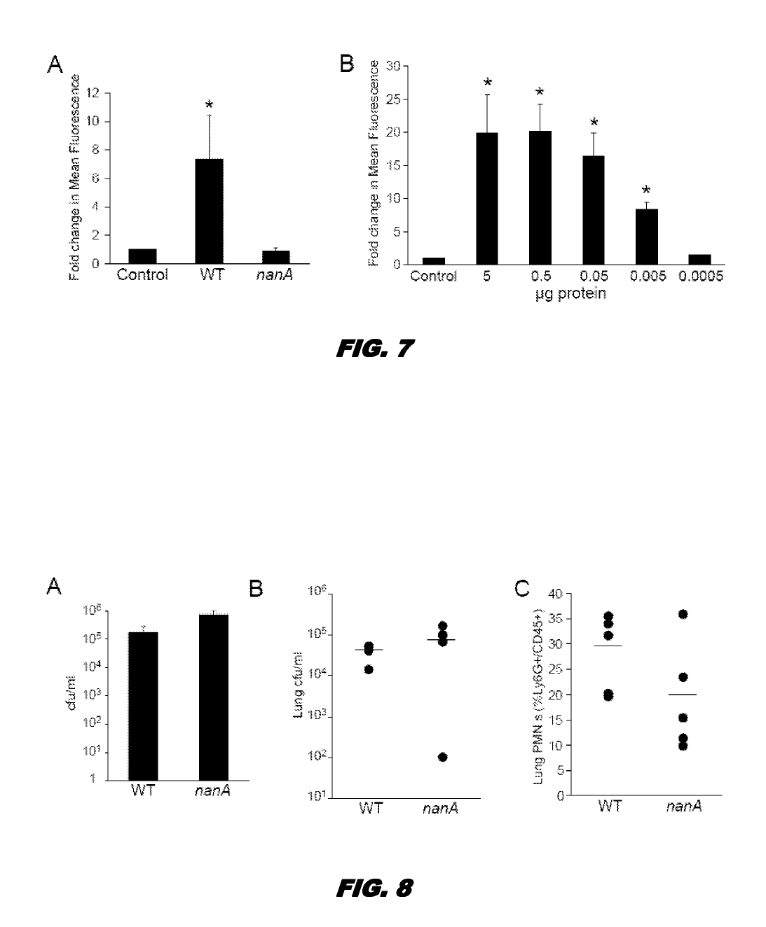

Biological activity of pneumococcal NanA. Many lung pathogens are able to bind to the asialylated ganglioside receptor GM1 (aGM1, Galβ1-3GalNAcβ1-4Galβ1-4Galβ1-Cer), including P. aeruginosa and S. pneumoniae [A22]. The neuraminidase from P. aeruginosa is able to expose this receptor [A47]; we sought to investigate if this was also the case for S. pneumoniae. Either purified NanA or concentrated supernatant from wild-type S. pneumoniae strain D39 but not its isogenic nanA mutant exposed aGM1 on the surface of 16HBE epithelial cells (FIG. 7). We then investigated the biological significance of this sialic acid release (FIG. 8). No effect on bacterial adherence was observed (FIG. 8A) nor was there a growth advantage in the presence of airway epithelial cells associated with the wild-type strain. Consistent with intratracheal infection studies [A29] and other colonization studies [A20], we did not observe a decrease in the ability for the nanA mutant to colonize mouse lungs after infection under anesthesia (FIG. 8B), although we did observe a trend towards less inflammation as assessed by neutrophil recruitment to the lung (FIG. 8C).

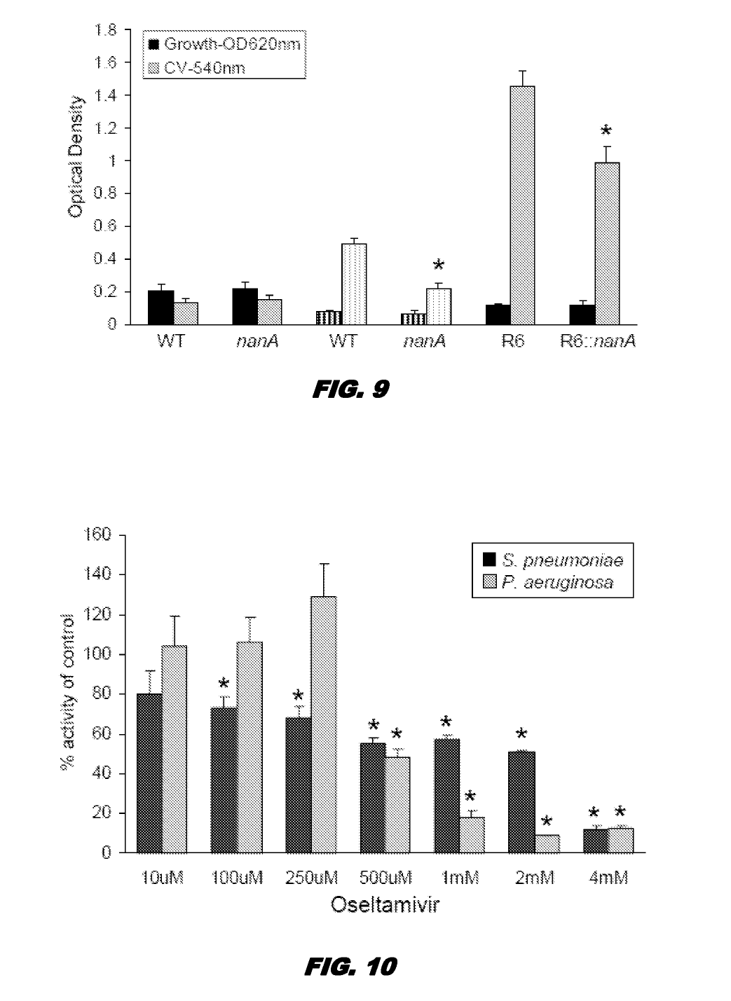

S. pneumoniae neuraminidase is involved in biofilm formation. A major function of the P. aeruginosa neuraminidase is its participation in cell-cell interactions necessary for biofilm formation [A37]. As S. pneumoniae nanA expression is upregulated in lung tissue and in biofilm-growing cells [A16] we investigated the contribution of nanA to the formation of biofilms (FIG. 9). Exposure of S. pneumoniae to airway epithelial cells caused a significant increase in biofilm formation and the nanA mutant had significantly reduced capacity to form biofilms. In an S. pneumoniae R6, unencapsulated background the nanA strain was also significantly reduced in its ability to form biofilms (FIG. 9). Of note, no difference in biofilm formation was observed when S. pneumoniae was grown on plastic without previous airway cell interaction.

Identification of small molecule inhibitors to the neuraminidases. Due to their comparable roles in the colonization and infection of the respiratory tract it seemed reasonable to predict that inhibitors targeting neuraminidases can prevent these respiratory infections. Inhibitors against the influenza neuraminidase, zanamivir (GSK) and oseltamivir (Roche) were both developed from structure-based design relying on high-resolution X-ray crystal structures and functional data [A3]. Oseltamivir was found to inhibit NanA in the micromolar to low millimolar range (FIG. 10), IC₅₀ 2 mM, a similar 50% inhibition was observed with NanPs at around 600 μM concentration (FIG. 10).

In an effort to identify small molecule inhibitors specific to the bacterial neuraminidases, a virtual library screen was performed (Schrödinger LLC, Portland, Oreg., USA) using the Glide software and the structure of NanPs [A53, A54]. The Glide analysis examines receptor-ligand docking, running a high-throughput screen of candidate compounds against the active site, a process that has been successful in identifying inhibitors of other enzymes [A55]. A number of compounds identified from this screen showed high degrees of inhibition in vitro, more-so with the pneumococcal enzyme which appears to behave more like a neuraminidase than P. aeruginosa (FIG. 11A). A lead compound, designated XX1, with a quinoline-based chemical scaffold that possesses appropriate pharmacokinetic properties for use in humans, such as adherence to the Lipinski rule of 5, was chosen for further analysis [A56]. XX1 was found to inhibit NanPs (FIG. 11B) and NanA (FIG. 11C) over a range of concentrations and in a dose-dependent manner. Consistent with the differences in the active site between the two enzymes we also observed differences in inhibition by XX1. An IC₅₀ of 8.5 μM was determined for the S. pneumoniae neuraminidase and 29 μM for the neuraminidase of P. aeruginosa. The inhibition afforded by XX1 to NanA is greater than 20, 80 and 200 times more effective than DANA, NANA and oseltamivir, respectively. Analogs of XX1 as well as a number of different chemical scaffolds in the development of effective inhibitors are currently undergoing further testing and development.

Discussion

The neuraminidases of S. pneumoniae and P. aeruginosa share a common role in facilitating bacterial colonization of the respiratory tract [A11, A29, A30, A37]. We extend this observation further, to show that both enzymes, despite differences in phylogeny and structure, also contribute to biofilm formation. For S. pneumoniae, the biofilm phenotype is most evident in vivo [A57], or as we show, under in vitro conditions that favor neuraminidase expression. We were only able to demonstrate a difference in biofilm production after growth on human airway cells, which induces nanA expression [A15, A16, A18] and reduces capsule production [A58]. By selecting for organisms on human airway cells, we apparently mimic an in vivo process in which encapsulated organisms that are able to avoid mucus entrapment and clearance gradually produce less capsule to facilitate epithelial attachment [A7]. Enhanced biofilm formation was also associated with the R6 strain, which exhibits reduced capsule production, with its nanA mutant also displaying reduced propensity for biofilm formation. The P. aeruginosa biofilm phenotype is similarly complex, involving several groups of genes involved in motility and the production of extracellular carbohydrates [A59, A60]. Like S. pneumoniae, the P. aeruginosa nanA mutant forms a minimal biofilm on human airway cells and is readily cleared from the murine upper airway [A37], consistent with a major role for the enzyme in the colonization process. As organisms enmeshed in extracellular carbohydrates are significantly more difficult to opsonize and phagocytose, the biofilm phenotype even early in pathogenesis appears critical for upper airway colonization.

Sialic acids represent a major component of the glycolipids that comprise the exposed surface of the respiratory mucosa. While sialic acids provide binding sites for pathogens such as influenza, desialylated glycolipids provide receptors for many of the common bacterial pulmonary pathogens including both S. pneumoniae and P. aeruginosa [A22], which bind to the exposed GalNAcβ1-4Gal residues when terminal sialic acid is cleaved. The desialylation of airway mucosal cells by the influenza neuraminidase increases susceptibility to secondary infection often caused by S. pneumoniae [A61]. We demonstrate that culture supernatant from wild-type but not the nanA mutant of S. pneumoniae is also capable of exposing asialylated glycolipids on human airway cells. However, the neur-aminidase activity associated with intact organisms, either S. pneumoniae or P. aeruginosa was not associated with increased bacterial attachment, indicating that the neuraminidase activity has a different biological role in pathogenesis. For S. pneumoniae, which is able to metabolize the carbohydrates released from airway mucins, neuraminidase activity can provide a growth advantage in vivo, although this has only been demonstrated in vitro [A27, A28]. Despite these differences, the observation that NanA and NanPs are both involved in biofilm formation highlights a common strategy of these two respiratory pathogens which need to persist in the same ecological niche. Despite their differences in structure, and different substrates, both neuraminidases possess similar functions in pathogenesis.

The structure of NanA shows that it is similar to canonical neuraminidases, with an active site that is a good fit for the NANA substrate. Thus, without being bound by theory, NanA behaves like a typical neuraminidase, and this is supported by our biochemical data. Experiences with developing inhibitors against other bacterial and viral neuraminidases can be beneficial for developing NanA inhibitors. In contrast, the structure of NanPs shows a distinct active site surface, such that NANA can no longer be tightly accommodated in it. Therefore, the biochemical function of NanPs can be different from that of NanA, and NanPs can also have a different inhibitor sensitivity profile as compared to NanA, as confirmed from our studies. Even subtle differences in neuraminidase structure can change the specificity for substrates, as seen with the Trypanosome enzymes [A62]. This is also consistent with our phylogenetic analysis, showing large evolutionary separation between NanPs and NanA. Moreover, NanPs contains a unique C-terminal domain, which also appears to be essential for its catalytic activity. The exact biological function of this domain, and in fact of NanPs, remains to be established.

The current inhibitors against influenza neuraminidase were developed through structure-function studies. We adapted this approach to identify a common inhibitor for the neuraminidases of P. aeruginosa and S. pneumoniae. Instead of starting with a base scaffold we undertook in silico ligand-receptor docking studies to identify putative inhibitors using high-resolution crystal structures before screening in our in vitro neuraminidase assays. This allowed us to screen and subsequently identify a range of chemical scaffolds that showed inhibition. The neuraminidase superfamily is quite divergent with low sequence identities, yet still retains tertiary structure similarities like those we have observed between these two neuraminidases [A4]. The differences in active site and probable differences in substrate between NanA and NanPs are also consistent with their divergent family clustering. While the differences between P. aeruginosa and S. pneumoniae were apparent in inhibitor screening as was the inhibition profile to oseltamivir, several inhibitors provided analogous levels of inhibition. This enabled us to develop a lead compound that shows significant inhibition against the two enzymes. We should be able to reduce the IC₅₀ for both enzymes further once we have conducted structure activity relationship studies or alternatively, develop the other chemical scaffolds that showed promise in the initial screen. Due to the active site differences between the two enzymes, individual compounds specific to each enzymes active site to increase efficacy need to be developed.

Although these enzymes are distant relatives on the phylogenetic tree, have different active site conformations and have relatively major differences in the affinities of their respective active sites for sialic acid, they share a common function in pathogenesis and the formation of biofilms. Based upon the structural data now available, we have begun to develop highly active neuraminidase inhibitors to prevent bacterial infection in the target populations at increased risk.

Materials and Methods

Bacterial strains and media. S. pneumoniae strains D39 [A63], D39 nanA [A19] and R6 [A64] and R6 nanA [A20] were grown on trypticase-soy (TS) agar or broth supplemented with 200 U/ml catalase (Worthington) and 1 μg/ml of chloramphenicol for nanA strains. Plate cultures were grown at 37° C. in the presence of carbon dioxide (5%). Escherichia coli strains were grown on Luria-Bertani (LB) media at 37° C., when required ampicillin was used at 100 μg/ml. All chemicals were purchased from Sigma unless otherwise stated.

Epithelial cell culture. Human bronchial epithelial cells, 16HBE and human airway cells, 1HAEo⁻ (Originally from D. Gruenert California Pacific Medical Center Research Institute, San Francisco, Calif., USA), were grown in minimum essential medium with Earle's salts (Cellgro and Gibco respectively) supplemented with 10% fetal bovine serum (Cambrex and Gibco respectively), 100 U/ml penicillin and 100 ug/ml streptomycin. 16HBE cells were additionally supplemented with 2 mM glutamine (Invitrogen). Cells were grown at 37° C. with 5% CO₂ in a humidified incubator.

Protein expression and purification. Full-length (residues 1-438) P. aeruginosa NanPs or residues 116-1035 of S. pneumoniae D39 sialidase A precursor (NanA) were sub-cloned into the pET28a vector (Novagen) and over-expressed in E. coli BL21(DE3) at 20° C. as N-terminal hexa-histidine tagged proteins. Site-directed mutations of the P. aeruginosa gene were performed using QuikChange (Stratagene), while the C-terminal truncated protein represents residues 1-333 (of 438). The soluble protein was purified by nickel-agarose, anion exchange and gel filtration chromatography. The P. aeruginosa protein was concentrated to 37 mg/ml, and S. pneumoniae protein to 30 mg/ml, in a solution containing 20 mM Tris (pH 8.5) and 200 mM NaCl, flash-frozen in liquid nitrogen in the presence of 5% (v/v) glycerol, and stored at −80° C. The N-terminal His-tag was not removed for crystallization. For the production of selenomethionyl proteins, the expression construct was transformed into B834 (DE3) cells (Novagen). The bacterial growth was carried out in defined LeMaster media supplemented with selenomethionine [A65] proteins purified following the same protocol as that for the native protein.

Protein crystallization. Crystals of NanPs free enzyme were obtained at 21° C. by the sitting-drop vapor diffusion method. The reservoir solution contained 100 mM Hepes (pH 7.0), 5% Tacsimate, 7% (w/v) PEG 5000MME, and the protein was at 10 mg/ml concentration. Selenomethionine-labeled protein was cross-microseeded with crystals from native protein to obtain adequate crystals. For data collection, the crystals were cryoprotected with the introduction of 20% (v/v) ethylene glycol, and flash-frozen in liquid nitrogen.

The initial crystallization screen of NanA did not produce any hits. We therefore performed limited proteolysis to search for a stable fragment. By treating NanA with trypsin at 1000:1 (protein:trypsin) ratio, a stable fragment of ˜50 kDa molecular weight was observed, indicating that approximately 50 kDa was removed from the recombinant protein by the trypsin treatment. Crystals of NanA were obtained by the sitting-drop vapor diffusion method. The reservoir solution contained 100 mM Hepes (pH 7.0) and 30% Jeffamine ED-2001 (pH 7.0), the protein was at 30 mg/ml and the drops also contained trypsin (at 5000:1 protein:trypsin ratio). Similar to our previous experiences with other proteins [A66, A67], in situ proteolysis was essential for the crystallization of this recombinant protein. We confirmed that the protein in the crystal showed smaller molecular weight (˜50 kDa) than the original expression construct (˜101 kDa) by SDS-PAGE, in agreement with our results from limited proteolysis with trypsin. For the complexes of NanA with NANA and DANA, crystals of the free enzyme were soaked overnight in a solution containing 100 mM Hepes (pH 7.0), 35% Jeffamine ED-2001 (pH 7.0), and 5 mM of each compound (NANA or DANA).

Data collection and processing. X-ray diffraction data were collected at the National Synchrotron Light Source (NSLS; Upton, N.Y., USA). Selenomethionyl single-wavelength anomalous diffraction (SAD) data sets at 1.9 Å and 1.7 Å resolution were collected for NanPs and NanA respectively. Native data sets at 1.6 Å resolution for NanPs and at 1.7 Å resolution for the NANA and DANA complexes of NanA were collected at the X4C beamline. The diffraction images were processed and scaled with the HKL package [A68]. The NanPs crystals belong to space group P2₁3, with cell parameters of a=b=c=125.6 Å. There is one molecule in the crystallographic asymmetric unit. The NanA crystals belong to the space group C2, with cell parameters of a=158.0 Å, b=47.7 Å, c=137.3 Å, and β=116.6°. There are two molecules in the crystallographic asymmetric unit.

Structure determination and refinement. The positions of Se atoms were determined with program BnP [A69]. Reflection phases were calculated based on the SAD data and improved with the program SOLVE/RESOLVE [A70], which also automatically located a large percentage of the residues in the molecule. The atomic model was fit into the electron density with the program O [A71]. The structure refinement was carried out with the program CNS [A72]. The crystallographic information is summarized in Table 1.

Neuraminidase assay. Neuraminidase activity of NanA was detected using the fluorogenic substrate 2′-(4-methylumbelliferyl)-α-D-N-acetylneuraminic acid (MNN, Sigma). Reactions contained 1.5 mM MNN, 1 nM of NanA in 2.5 mM sodium phosphate buffer (pH 5). Reactions were allowed to incubate for 2 h at 37° C. before fluorescence intensity was measured at excitation and emission wavelengths of 360 nm and 465 nm in a Tecan microplate reader (Männedorf, Switzerland). NanPs was assayed as above with 1 mM of enzyme in 7.5 mM sodium chloride and 4 mM calcium chloride. Compounds were screened at a concentration of 100 μM before dose-dependence was established. Compounds were obtained from a variety of sources (Otava, Kyiv, Ukraine; Interbioscreen, Moscow, Russia; Chembridge San Diego, USA; Maybridge, Cornwell, UK; Sigma, St Louis, USA; Princeton, Princeton, USA; Lifechem, Burlington, Canada; Enamine, Kiev, Ukraine). Neuraminidase assays with oseltamivir were performed using the hydrolyzed version of the compound. Divalent cations were supplied in the form of calcium, magnesium, ferric and copper chloride. All neuraminidase assays were performed at least in triplicate.

Quantification of asialoGM1 exposure by flow cytometry. 16HBE cells were grown in 24-well plates to confluence and exposed to bacterial supernatants for 3 h followed by three PBS washes. Supernatants were concentrated approximately 30-fold (Amicon Ultra, Millipore) and adjusted for protein quantity. As a control media alone was also concentrated. Cells were stained with rabbit polyclonal anti aGM1 antibody (Wako) followed by Alexa Fluor 488 donkey anti-rabbit IgG (Molecular Probes). Cells were detached from the plates using 0.02% EGTA in HBSS (Hanks buffered saline solution), fixed with 1% paraformaldehyde and analyzed on a FACSCalibur using CellQuest software (version 3.3; BD). Data were analyzed using WinMDI (version 2.8, Joseph Trotter).

Adherence assay. Adherence assays were performed using 16HBE cells. Bacterial strains were grown to mid-log phase, washed with PBS and 0.7-2×10⁷ cfu of bacterial cells were added to confluent monolayers in 24-well plates. Bacterial cells were allowed to adhere for 1 h at 37° C. before three washes with PBS. Bacteria were dissociated from epithelial cells using TrypLE Express (Gibco) and plated out to determine adherent numbers. The assay was performed with three biological replicates with duplicate technical replicates over two separate experiments.

Biofilm assay. Bacterial strains were grown to mid-log phase before being diluted 1:100 in TS broth and catalase. 100 μl of diluted culture was added in triplicate to 96-well flat bottom tissue culture treated plates (Falcon) and left for 18-24 h at 37° C. with 5% CO₂. Plates were read at 600 nm to determine levels of growth before being washed in water. Adherent biofilm-forming cells were then stained with 125 μL of 1% crystal violet for 15 min before two further washes in water and allowed to dry. Bound crystal violet was then suspended in 200 μL of ethanol, shaken for 15 min and read at 540 nm.

Biofilm formation after epithelial cell interaction. Bacterial strains were grown and inoculated onto 1HAEo⁻ cells as per adherence assay. After the initial PBS washes fresh MEM media was applied before a further 1 h incubation. This removal and addition of fresh media was repeated a further four times before detachment of adherent bacteria using TrypLE Express (Gibco). The detached bacteria were then diluted 1:100 in TS broth and catalase and assayed as per the biofilm assay. Samples were repeated four times, each time in sextuplicate using epithelial cells without bacteria as a negative control.

Mouse model of pneumonia. Six-week-old C57Bl/6J mice (Jackson Laboratories) were anaesthetized with ketamine (100 mg/kg) and xylazine (5 mg/kg) before intranasal inoculation with bacteria. Bacteria were grown to mid-log phase, washed with PBS and 3−4×10⁵ cfu inoculated down the nare in a 10 μL volume. The infection persisted for 24 h before euthanasia with pentobarbital. Lungs were removed, homogenized, and plated out to determine bacterial numbers representative of pneumonia and bacteremia respectively. A portion of the lung homogenate was double-stained with phycoerythrin-labeled anti-CD45 and FITC-labeled anti-Ly6G antibodies to determine neutrophil influx into the lung by flow cytometry. Irrelevant, matched isotope antibodies were used as controls. Cells were gated based on forward and site scatter with neutrophils expressed as the Ly6G positive population within CD45 positive cells.

Phylogenetic analysis. Our sampling strategy was aimed at maximizing phylogenetic breadth in order to understand the overall pattern of evolution in the neuraminidase/sialidase gene family. We began with a list of well-known neuraminidases including those from V. cholerae, S. typhimurium, C. perfringens, S. pneumoniae, T. cruzi, and P. aeruginosa. For each sequence we did a standard BLAST search and collected one sequence from each genus in the list of hits that had an e-value score of 1×10⁻⁵ or lower. Duplicates were deleted. We also included sequences that have been included in other prior publications on the evolution of sialidases [A4]. A list of sequences and gene identification numbers is included as supplemental information. Sequences were aligned using the ClustalW algorithm as implemented in the program BioEdit using default settings. Sequences were aligned as amino acids and then transposed back to the original nucleotide sequences maintaining the gaps determined by the initial alignment (5394 characters total, 4124 parsimony informative characters with gaps as a fifth state, 3766 parsimony informative characters with gaps as treated as missing).

A rigorous phylogenetic analysis was undertaken using maximum parsimony (MP) algorithm implemented in PAUP* [A73]. 1000 replicates of random addition (RA) was performed followed by the tree branch reconnection (TBR) algorithm using the “multrees” option to save more than one optimal tree if discovered in the search. All characters and state transformations were given equal weight. Terminal gaps were scored as missing data in all analyses. We performed two analyses designating internal gaps as a fifth character state in one and as missing data in the other. Although the trees had many nodes in agreement there were major differences between the structures of the 2 trees. Since gaps can be informative characters [A74, A75], analysis in which internal gaps are counted as character states were used. Nonparametric bootstrap analyses was performed with 100 iterations consisting of 100 RA replicates followed by TBR to gauge the robustness of the tree.

Statistics. Significance of data was determined either using a students t-test or non-parametric Mann-Whitney test using GraphPad Prism software.

Accession numbers. The GenBank (www.ncbi.nlm.nih.gov/Entrez/) accession numbers utilized for phylogenetic analysis are: Verrucomicrobium spinosum gi|164421336:353068-354225, Blastopirellula marina gi|87311313:89394-90503, Lentisphaera araneosa gi|149198907:89577-90743, Propionibacterium acnes gi|50841496:752060-754375, Ruminococcus lactaris gi|197302028:32228-35857, Erysipelothrix rhusiopathiae gi|13516389:295-3807, Pasteurella multocida gi|15601865:1176085-1179327, Actinomyces odontolyticus gi|145845834:308755-310992, Mannheimia haemolytica gi|125433996:1-2376, Haemophilus parasuis gi|167854877:54475-56886, Bacteroides fragilis gi|53711291:4836372-4838006, Akkermansia muciniphila gi|187734516:2229943-2231967, Capnocytophaga canimorsus gi|194454827:2197-3765, Parabacteroides distasonis gi|150006674:3525685-3527310, Shewanella pealeana gi|157959830:1838982-1841831, Flavobacteriales bacterium gi|88710837:680637-681797, Rhodopirellula baltica gi|32470666:1724290-1725519, Opitutaceae bacterium gi|153892517:3249-4847, Sassharopolyspora erythraea gi|134096620:5769332-5771182, Pseudoalteromonas haloplanktis gi|77361923:196316-197458, Chthoniobacter flavus gi|196231426:66519-67730, Janibacter sp. gi|84494251:767782-770736, Monosiga brevicollis gi|167534964:1-984, Strongylocentrotus purpuratus gi|115616575:1-719, Planctomyces maris gi|149177549:10030-11205, Acinetobacter baumannii gi|169632029:647539-649371, Opitutaceae bacterium gi|153890920:5481-6641, Danio rerio gi|148539964:69-1220, Corynebacterium diptheriae gi|38232642:512872-515037, Gemmata obscuriglobus gi|163804184:63331-64515, Streptomyces coelicolor gi132141095:7255596-7257542, Takifugu rubripes gi|148372013:1-87, V. cholerae gi|12057212:1933231-1935654, C. perfringens nanI gi|18308982:900997-903081, C. perfringens nanH gi|18308982:904693-905499, P. aeruginosa gi|110227054:3150886-3152202, C. septicum gi140662, C. sordellii gi|1710442, A. viscosus gi|39254, Trypanosoma rangeli gi|2894809, T. cruzi gi|162265, T. cruzi SAPA (shed-acute-phase-antigen) gi|10943, Micromonosporta viridifaciens gi|216782:816-2759, Arthrobacter ureafaciens gi|60544840, Influenzae A H5N1 gi|108671038, Macrobdella decora gi|1353880, Salmonella typhimurium gi|16763390:1002088-1003326, S. pneumoniae gi|116515308:1522475-1525468, Arcanobacterium pyogenes gi|18146340:1026-6239, Xenopus laevis gi|148228846:180-1376, Trichomonas vaginalis gi|123473002:1-1050, Rattus norvegicus gi|71896601:59-1288, Bos taurus gi|149676185:61-219, 650-842, 1541-1803, 2490-2672, 2849-3071, 3185-3411, and Monodelphis domestica gi|126309689:1-1404.

### REFERENCES

A1. Corfield T (1992) Bacterial sialidases—roles in pathogenicity and nutrition. Glycobiology 2: 509-521.
A2. Taylor G (1996) Sialidases: structures, biological significance and therapeutic potential. Curr Opin Struct Biol 6: 830-837.
A3. von Itzstein M (2007) The war against influenza: discovery and development of sialidase inhibitors. Nat Rev Drug Discov 6: 967-974.
A4. Roggentin P, Schauer R, Hoyer L L, Vimr E R (1993) The sialidase superfamily and its spread by horizontal gene transfer. Mol Microbiol 9: 915-921.
A5. World Health Organization (2007) Pneumococcal conjugate vaccine for childhood immunization—WHO position paper. Weekly epidemiological record 82: 93-104.
A6. Miser W F (2001) To treat or not to treat otitis media—that's just one of the questions. J Am Board Fam Pract 14: 474-476.
A7. Kadioglu A, Weiser J N, Paton J C, Andrew P W (2008) The role of Streptococcus pneumoniae virulence factors in host respiratory colonization and disease. Nat Rev Microbiol 6: 288-301.
A8. Brueggemann A B, Pai R, Crook D W, Beall B (2007) Vaccine escape recombinants emerge after pneumococcal vaccination in the United States. PLoS Pathog 3: e168.
A9. Singleton R J, Hennessy T W, Bulkow L R, Hammitt L L, Zulz T, et al. (2007) Invasive pneumococcal disease caused by nonvaccine serotypes among alaska native children with high levels of 7-valent pneumococcal conjugate vaccine coverage. Jama 297: 1784-1792.
A10. Pettigrew M M, Fennie K P, York M P, Daniels J, Ghaffar F (2006) Variation in the presence of neuraminidase genes among Streptococcus pneumoniae isolates with identical sequence types. Infect Immun 74: 3360-3365.
A11. Manco S, Hernon F, Yesilkaya H, Paton J C, Andrew P W, et al. (2006) Pneumococcal neuraminidases A and B both have essential roles during infection of the respiratory tract and sepsis. Infect Immun 74: 4014-4020.
A12. Berry A M, Lock R A, Paton J C (1996) Cloning and characterization of nanB, a second Streptococcus pneumoniae neuraminidase gene, and purification of the NanB enzyme from recombinant Escherichia coli. J Bacteriol 178: 4854-4860.
A13. Kelly R T, Farmer S, Greiff D (1967) Neuraminidase activities of clinical isolates of Diplococcus pneumoniae. J Bacteriol 94: 272-273.
A14. King S J, Whatmore A M, Dowson C G (2005) NanA, a neuraminidase from Streptococcus pneumoniae, shows high levels of sequence diversity, at least in part through recombination with Streptococcus oralis. J Bacteriol 187: 5376-5386.
A15. Song X M, Connor W, Hokamp K, Babiuk L A, Potter A A (2008) Streptococcus pneumoniae early response genes to human lung epithelial cells. BMC Res Notes 1: 64.
A16. Oggioni M R, Trappetti C, Kadioglu A, Cassone M, Iannelli F, et al. (2006) Switch from planktonic to sessile life: a major event in pneumococcal pathogenesis. Mol Microbiol 61: 1196-1210.
A17. Williamson Y M, Gowrisankar R, Longo D L, Facklam R, Gipson I K, et al. (2008) Adherence of nontypeable Streptococcus pneumoniae to human conjunctival epithelial cells. Microb Pathog 44: 175-185.
A18. LeMessurier K S, Ogunniyi A D, Paton J C (2006) Differential expression of key pneumococcal virulence genes in vivo. Microbiology 152: 305-311.
A19. King S J, Hippe K R, Gould J M, Bae D, Peterson S, et al. (2004) Phase variable desialylation of host proteins that bind to Streptococcus pneumoniae in vivo and protect the airway. Mol Microbiol 54: 159-171.
A20. King S J, Hippe K R, Weiser J N (2006) Deglycosylation of human glycoconjugates by the sequential activities of exoglycosidases expressed by Streptococcus pneumoniae. Mol Microbiol 59: 961-974.
A21. Linder T E, Daniels R L, Lim D J, DeMaria T F (1994) Effect of intranasal inoculation of Streptococcus pneumoniae on the structure of the surface carbohydrates of the chinchilla eustachian tube and middle ear mucosa. Microb Pathog 16: 435-441.
A22. Krivan H C, Roberts D D, Ginsburg V (1988) Many pulmonary pathogenic bacteria bind specifically to the carbohydrate sequence GalNAc beta 1-4Gal found in some glycolipids. Proc Natl Acad Sci USA 85: 6157-6161.
A23. Andersson B, Dahmen J, Frejd T, Leffler H, Magnusson G, et al. (1983) Identification of an active disaccharide unit of a glycoconjugate receptor for pneumococci attaching to human pharyngeal epithelial cells. J Exp Med 158: 559-570.
A24. Tong H H, McIver M A, Fisher L M, DeMaria T F (1999) Effect of lacto-N-neotetraose, asialoganglioside-GM1 and neuraminidase on adherence of otitis media-associated serotypes of Streptococcus pneumoniae to chinchilla tracheal epithelium. Microb Pathog 26: 111-119.
A25. Tong H H, Liu X, Chen Y, James M, Demaria T (2002) Effect of neuraminidase on receptor-mediated adherence of Streptococcus pneumoniae to chinchilla tracheal epithelium. Acta Otolaryngol 122: 413-419.
A26. Shakhnovich E A, King S J, Weiser J N (2002) Neuraminidase expressed by Streptococcus pneumoniae desialylates the lipopolysaccharide of Neisseria meningitidis and Haemophilus influenzae: a paradigm for interbacterial competition among pathogens of the human respiratory tract. Infect Immun 70: 7161-7164.
A27. Burnaugh A M, Frantz L J, King S J (2008) Growth of Streptococcus pneumoniae on human glycoconjugates is dependent upon the sequential activity of bacterial exoglycosidases. J Bacteriol 190: 221-230.
A28. Yesilkaya H, Manco S, Kadioglu A, Terra V S, Andrew P W (2008) The ability to utilize mucin affects the regulation of virulence gene expression in Streptococcus pneumoniae. FEMS Microbiol Lett 278: 231-235.
A29. Orihuela C J, Gao G, Francis K P, Yu J, Tuomanen E I (2004) Tissue-specific contributions of pneumococcal virulence factors to pathogenesis. J Infect Dis 190: 1661-1669.
A30. Tong H H, Blue L E, James M A, DeMaria T F (2000) Evaluation of the virulence of a Streptococcus pneumoniae neuraminidase-deficient mutant in nasopharyngeal colonization and development of otitis media in the chinchilla model. Infect Immun 68: 921-924.
A31. Lock R A, Paton J C, Hansman D (1988) Comparative efficacy of pneumococcal neuraminidase and pneumolysin as immunogens protective against Streptococcus pneumoniae. Microb Pathog 5: 461-467.
A32. Long J P, Tong H H, DeMaria T F (2004) Immunization with native or recombinant Streptococcus pneumoniae neuraminidase affords protection in the chinchilla otitis media model. Infect Immun 72: 4309-4313.
A33. Tong H H, Li D, Chen S, Long J P, DeMaria T F (2005) Immunization with recombinant Streptococcus pneumoniae neuraminidase NanA protects chinchillas against nasopharyngeal colonization. Infect Immun 73: 7775-7778.
A34. Berry A M, Paton J C (2000) Additive attenuation of virulence of Streptococcus pneumoniae by mutation of the genes encoding pneumolysin and other putative pneumococcal virulence proteins. Infect Immun 68: 133-140.
A35. Gingles N A, Alexander J E, Kadioglu A, Andrew P W, Kerr A, et al. (2001) Role of genetic resistance in invasive pneumococcal infection: identification and study of susceptibility and resistance in inbred mouse strains. Infect Immun 69: 426-434.
A36. Sadikot R T, Blackwell T S, Christman J W, Prince A S (2005) Pathogen-host interactions in Pseudomonas aeruginosa pneumonia. Am J Respir Crit. Care Med 171: 1209-1223.
A37. Soong G, Muir A, Gomez M I, Waks J, Reddy B, et al. (2006) Bacterial neuraminidase facilitates mucosal infection by participating in biofilm production. J Clin Invest 116: 2297-2305.
A38. Lanotte P, Watt S, Mereghetti L, Dartiguelongue N, Rastegar-Lari A, et al. (2004) Genetic features of Pseudomonas aeruginosa isolates from cystic fibrosis patients compared with those of isolates from other origins. J Med Microbiol 53: 73-81.
A39. Crennell S J, Garman E F, Philippon C, Vasella A, layer W G, et al. (1996) The structures of Salmonella typhimurium LT2 neuraminidase and its complexes with three inhibitors at high resolution. J Mol Biol 259: 264-280.
A40. Crennell S, Garman E, layer G, Vimr E, Taylor G (1994) Crystal structure of Vibrio cholerae neuraminidase reveals dual lectin-like domains in addition to the catalytic domain. Structure 2: 535-544.
A41. Newstead S L, Potter J A, Wilson J C, Xu G, Chien C H, et al. (2008) The structure of Clostridium perfringens NanI sialidase and its catalytic intermediates. J Biol Chem 283: 9080-9088.
A42. Gaskell A, Crennell S, Taylor G (1995) The three domains of a bacterial sialidase: a beta-propeller, an immunoglobulin module and a galactose-binding jelly-roll. Structure 3: 1197-1205.
A43. Xu G, Li X, Andrew P W, Taylor G L (2008) Structure of the catalytic domain of Streptococcus pneumoniae sialidase NanA. Acta Cryst F64: 772-775.
A44. Gut H, King S J, Walsh M A (2008) Structural and functional studies of Streptococcus pneumoniae neuraminidase B: An intramolecular trans-sialidase. FEBS Lett.
A45. Krissinel E, Henrick K (2004) Secondary-structure matching (SSM), a new tool for fast protein structure alignment in three dimensions. Acta Cryst D60: 2256-2268.
A46. Fong S, Hamill S J, Proctoer M, Freund S M V, Benian G M, et al. (1996) Structure and stability of an immunoglobulin superfamily domain from twitchin, a muscle protein of the nematode Caenorhabditis elegans. J Mol Biol 264: 624-639.
A47. Cacalano G, Kays M, Saiman L, Prince A (1992) Production of the Pseudomonas aeruginosa neuraminidase is increased under hyperosmolar conditions and is regulated by genes involved in alginate expression. J Clin Invest 89: 1866-1874.
A48. Carr J, Ives J, Kelly L, Lambkin R, Oxford J, et al. (2002) Influenza virus carrying neuraminidase with reduced sensitivity to oseltamivir carboxylate has altered properties in vitro and is compromised for infectivity and replicative ability in vivo. Antiviral Res 54: 79-88.
A49. Wang Q, Chang B J, Mee B J, Riley T V (2005) Neuraminidase production by Erysipelothrix rhusiopathiae. Vet Microbiol 107: 265-272.
A50. Schlesner H, Rensmann C, Tindall B J, Gade D, Rabus R, et al. (2004) Taxonomic heterogeneity within the Planctomycetales as derived by DNA-DNA hybridization, description of Rhodopirellula baltica gen. nov., sp. nov., transfer of Pirellula marina to the genus Blastopirellula gen. nov. as Blastopirellula marina comb. nov. and emended description of the genus Pirellula. Int J Syst Evol Microbiol 54: 1567-1580.
A51. Holmquist L (1975) Activation of Vibrio cholerae neuraminidase by divalent cations. FEBS Lett 50: 269-271.
A52. Holzer C T, von Itzstein M, Jin B, Pegg M S, Stewart W P, et al. (1993) Inhibition of sialidases from viral, bacterial and mammalian sources by analogues of 2-deoxy-2,3-didehydro-N-acetylneuraminic acid modified at the C-4 position. Glycoconj J 10: 40-44.
A53. Sherman W, Day T, Jacobson M P, Friesner R A, Farid R (2006) Novel procedure for modeling ligand/receptor induced fit effects. J Med Chem 49: 534-553.
A54. Kontoyianni M, McClellan L M, Sokol G S (2004) Evaluation of docking performance: comparative data on docking algorithms. J Med Chem 47: 558-565.
A55. Siddiquee K, Zhang S, Guida W C, Blaskovich M A, Greedy B, et al. (2007) Selective chemical probe inhibitor of Stat3, identified through structure-based virtual screening, induces antitumor activity. Proc Natl Acad Sci USA 104: 7391-7396.
A56. Lipinski C A, Lombardo F, Dominy B W, Feeney P J (2001) Experimental and computational approaches to estimate solubility and permeability in drug discovery and development settings. Adv Drug Deliv Rev 46: 3-26.
A57. Hall-Stoodley L, Hu F Z, Gieseke A, Nistico L, Nguyen D, et al. (2006) Direct detection of bacterial biofilms on the middle-ear mucosa of children with chronic otitis media. Jama 296: 202-211.
A58. Hammerschmidt S, Wolff S, Hocke A, Rosseau S, Muller E, et al. (2005) Illustration of pneumococcal polysaccharide capsule during adherence and invasion of epithelial cells. Infect Immun 73: 4653-4667.
A59. Ryder C, Byrd M, Wozniak D J (2007) Role of polysaccharides in Pseudomonas aeruginosa biofilm development. Curr Opin Microbiol 10: 644-648.
A60. Kirisits M J, Parsek M R (2006) Does Pseudomonas aeruginosa use intercellular signalling to build biofilm communities? Cell Microbiol 8: 1841-1849.
A61. McCullers J A (2006) Insights into the interaction between influenza virus and pneumococcus. Clin Microbiol Rev 19: 571-582.
A62. Amaya M F, Buschiazzo A, Nguyen T, Alzari P M (2003) The high resolution structures of free and inhibitor-bound Trypanosoma rangeli sialidase and its comparison with T. cruzi trans-sialidase. J Mol Biol 325: 773-784.
A63. Avery O T, MacLeod C M, McCarty M (1944) Studies on the chemical nature of the substance inducing transformation of pneumococcal types: inductions of transformation by a desoxyribonucleic acid fraction isolated from pneumococcus type III. J Exp Med 79: 137-158.
A64. Hoskins J, Alborn W E, Jr., Arnold J, Blaszczak L C, Burgett S, et al. (2001) Genome of the bacterium Streptococcus pneumoniae strain R6. J Bacteriol 183: 5709-5717.
A65. Hendrickson W A, Horton J R, LeMaster D M (1990) Selenomethionyl proteins produced for analysis by multiwavelength anomalous diffraction (MAD): a vehicle for direct determination of three-dimensional structure. EMBO J. 9: 1665-1672.
A66. Mandel C R, Gebauer D, Zhang H, Tong L (2006) A serendipitous discovery that in situ proteolysis is required for the crystallization of yeast CPSF-100 (Ydh1p). Acta Cryst F62: 1041-1045.
A67. Bai Y, Auperin T C, Tong L (2007) The use of in situ proteolysis in the crystallization of murine CstF-77. Acta Cryst F63: 135-138.
A68. Otwinowski Z, Minor W (1997) Processing of x-ray diffraction data collected in oscillation mode. Methods in Enzymology 276: 307-326.
A69. Weeks C M, Miller R (1999) The design and implementation of SnB v2.0. J Appl Cryst 32: 120-124.
A70. Terwilliger T C (2003) SOLVE and RESOLVE: Automated structure solution and density modification. Meth Enzymol 374: 22-37.
A71. Jones T A, Zou J Y, Cowan S W, Kjeldgaard M (1991) Improved methods for building protein models in electron density maps and the location of errors in these models. Acta Crystallogr A 47 (Pt 2): 110-119.
A72. Brunger A T, Adams P D, Clore G M, DeLano W L, Gros P, et al. (1998) Crystallography & NMR system: A new software suite for macromolecular structure determination. Acta Crystallogr D Biol Crystallogr 54: 905-921.
A73. Swofford D L (1998) PAUP*: Phylogenetic analysis using parsimony (*and other methods). 4.0 beta ed. Sunderland, Massachussetts: Sinauer.
A74. Giribet G, Wheeler W C (1999) On gaps. Mol Phylogenet Evol 13: 132-143.
A75. Phillips A, Janies D, Wheeler W (2000) Multiple sequence alignment in phylogenetic analysis. Mol Phylogenet Evol 16: 317-330.
A76. DeLano W L (2002) The PyMOL Molecular Graphics System. San Carlos, Calif.: DeLano Scientific.
A77. Nicholls A, Sharp K A, Honig B (1991) Protein folding and association: insights from the interfacial and thermodynamic properties of hydrocarbons. Proteins 11: 281-296.

### Example 2

### In Silico Screening

In silico screening. To demonstrate the feasibility of identifying candidate molecule inhibitors, in silico, computational modeling software can be utilized in conjunction with high-resolution crystal structure results (for example, using the X-ray coordinates of Table 2) to screen databases for existing compounds that would bind to the active site of the Streptococcus neuraminidase (see Sherman et al., (2006) Chem Biol Drug Des 67(1): 83-4). The atomic coordinates for residues of a S. pneumoniae neuraminidase crystal were obtained from PDB Structure No. 2vvZ (available at, http://www.pdb.org/pdb/explore.do? structureId=2VVZ).

TABLE 2
Atomic Coordinates for S. pneumoniae Neuraminidase Crystal. Table
2 discloses SEQ ID NOS: 3 and 4, respectively.

| | | | | | | | | |
|---|---|---|---|---|---|---|---|---|
| CRYST1 | 49.206 | 95.626 | 226.596 | 90.00 | 90.00 | 90.00 | P 21 21 21 | 8 |

| | | | | | | | | | | | |
|---|---|---|---|---|---|---|---|---|---|---|---|
| ATOM | 1 | N | ALA | A | 322 | 16.926 | 39.763 | 36.357 | 1.00 | 30.98 | N |
| ATOM | 2 | CA | ALA | A | 322 | 18.021 | 40.720 | 36.692 | 1.00 | 31.11 | C |
| ATOM | 3 | C | ALA | A | 322 | 18.342 | 40.708 | 38.189 | 1.00 | 31.33 | C |
| ATOM | 4 | O | ALA | A | 322 | 17.520 | 40.297 | 39.013 | 1.00 | 31.21 | O |
| ATOM | 5 | CB | ALA | A | 322 | 17.669 | 42.134 | 36.222 | 1.00 | 31.03 | C |
| ATOM | 6 | N | LEU | A | 323 | 19.546 | 41.175 | 38.517 | 1.00 | 31.48 | N |
| ATOM | 7 | CA | LEU | A | 323 | 20.086 | 41.158 | 39.870 | 1.00 | 31.45 | C |
| ATOM | 8 | C | LEU | A | 323 | 20.926 | 42.428 | 40.038 | 1.00 | 31.70 | C |
| ATOM | 9 | O | LEU | A | 323 | 21.666 | 42.800 | 39.128 | 1.00 | 31.75 | O |
| ATOM | 10 | CB | LEU | A | 323 | 20.941 | 39.896 | 40.060 | 1.00 | 31.28 | C |
| ATOM | 11 | CG | LEU | A | 323 | 21.781 | 39.620 | 41.314 | 1.00 | 31.31 | C |
| ATOM | 12 | CD1 | LEU | A | 323 | 20.920 | 39.323 | 42.525 | 1.00 | 29.41 | C |
| ATOM | 13 | CD2 | LEU | A | 323 | 22.738 | 38.460 | 41.055 | 1.00 | 31.25 | C |
| ATOM | 14 | N | THR | A | 324 | 20.804 | 43.098 | 41.182 | 1.00 | 31.95 | N |
| ATOM | 15 | CA | THR | A | 324 | 21.581 | 44.320 | 41.430 | 1.00 | 32.37 | C |
| ATOM | 16 | C | THR | A | 324 | 23.043 | 44.005 | 41.748 | 1.00 | 32.57 | C |
| ATOM | 17 | O | THR | A | 324 | 23.379 | 42.879 | 42.113 | 1.00 | 32.43 | O |
| ATOM | 18 | CB | THR | A | 324 | 20.982 | 45.190 | 42.567 | 1.00 | 32.46 | C |
| ATOM | 19 | OG1 | THR | A | 324 | 20.949 | 44.444 | 43.796 | 1.00 | 32.85 | O |
| ATOM | 20 | CG2 | THR | A | 324 | 19.580 | 45.667 | 42.203 | 1.00 | 32.29 | C |
| ATOM | 21 | N | GLU | A | 325 | 23.914 | 44.997 | 41.583 | 1.00 | 32.98 | N |
| ATOM | 22 | CA | GLU | A | 325 | 25.307 | 44.854 | 42.004 | 1.00 | 33.36 | C |
| ATOM | 23 | C | GLU | A | 325 | 25.321 | 44.853 | 43.524 | 1.00 | 32.91 | C |
| ATOM | 24 | O | GLU | A | 325 | 24.356 | 45.293 | 44.160 | 1.00 | 32.79 | O |
| ATOM | 25 | CB | GLU | A | 325 | 26.194 | 45.986 | 41.463 | 1.00 | 33.55 | C |
| ATOM | 26 | CG | GLU | A | 325 | 25.966 | 46.359 | 39.991 | 1.00 | 35.86 | C |
| ATOM | 27 | CD | GLU | A | 325 | 25.949 | 45.156 | 39.048 | 1.00 | 38.13 | C |
| ATOM | 28 | OE1 | GLU | A | 325 | 26.927 | 44.374 | 39.044 | 1.00 | 39.16 | O |
| ATOM | 29 | OE2 | GLU | A | 325 | 24.957 | 45.002 | 38.300 | 1.00 | 38.96 | O |
| ATOM | 30 | N | LYS | A | 326 | 26.400 | 44.347 | 44.109 | 1.00 | 32.46 | N |
| ATOM | 31 | CA | LYS | A | 326 | 26.471 | 44.286 | 45.560 | 1.00 | 32.04 | C |
| ATOM | 32 | C | LYS | A | 326 | 26.632 | 45.671 | 46.184 | 1.00 | 31.69 | C |
| ATOM | 33 | O | LYS | A | 326 | 27.401 | 46.506 | 45.706 | 1.00 | 31.56 | O |
| ATOM | 34 | CB | LYS | A | 326 | 27.534 | 43.280 | 46.048 | 1.00 | 31.93 | C |
| ATOM | 35 | CG | LYS | A | 326 | 28.980 | 43.619 | 45.753 | 1.00 | 32.00 | C |
| ATOM | 36 | CD | LYS | A | 326 | 29.899 | 42.521 | 46.273 | 1.00 | 32.02 | C |
| ATOM | 37 | CE | LYS | A | 326 | 31.350 | 42.984 | 46.340 | 1.00 | 31.95 | C |
| ATOM | 38 | NZ | LYS | A | 326 | 31.616 | 43.884 | 47.501 | 1.00 | 31.51 | N |
| ATOM | 39 | N | THR | A | 327 | 25.841 | 45.919 | 47.220 | 1.00 | 31.37 | N |
| ATOM | 40 | CA | THR | A | 327 | 25.969 | 47.110 | 48.040 | 1.00 | 30.95 | C |
| ATOM | 41 | C | THR | A | 327 | 26.493 | 46.624 | 49.387 | 1.00 | 30.70 | C |
| ATOM | 42 | O | THR | A | 327 | 25.834 | 45.830 | 50.063 | 1.00 | 30.83 | O |
| ATOM | 43 | CB | THR | A | 327 | 24.611 | 47.824 | 48.191 | 1.00 | 30.88 | C |
| ATOM | 44 | OG1 | THR | A | 327 | 24.012 | 47.976 | 46.900 | 1.00 | 31.31 | O |
| ATOM | 45 | CG2 | THR | A | 327 | 24.771 | 49.194 | 48.818 | 1.00 | 30.55 | C |
| ATOM | 46 | N | ASP | A | 328 | 27.693 | 47.068 | 49.752 | 1.00 | 30.24 | N |
| ATOM | 47 | CA | ASP | A | 328 | 28.331 | 46.641 | 50.999 | 1.00 | 29.79 | C |
| ATOM | 48 | C | ASP | A | 328 | 27.777 | 47.416 | 52.192 | 1.00 | 29.68 | C |
| ATOM | 49 | O | ASP | A | 328 | 27.874 | 48.642 | 52.253 | 1.00 | 29.82 | O |
| ATOM | 50 | CB | ASP | A | 328 | 29.857 | 46.776 | 50.911 | 1.00 | 29.49 | C |
| ATOM | 51 | CG | ASP | A | 328 | 30.466 | 45.925 | 49.797 | 1.00 | 28.84 | C |
| ATOM | 52 | OD1 | ASP | A | 328 | 29.844 | 44.938 | 49.350 | 1.00 | 27.94 | O |
| ATOM | 53 | OD2 | ASP | A | 328 | 31.587 | 46.240 | 49.364 | 1.00 | 29.00 | O |
| ATOM | 54 | N | ILE | A | 329 | 27.196 | 46.687 | 53.137 | 1.00 | 29.47 | N |
| ATOM | 55 | CA | ILE | A | 329 | 26.494 | 47.288 | 54.267 | 1.00 | 29.07 | C |
| ATOM | 56 | C | ILE | A | 329 | 27.360 | 47.296 | 55.518 | 1.00 | 28.81 | C |
| ATOM | 57 | O | ILE | A | 329 | 27.310 | 48.232 | 56.312 | 1.00 | 28.75 | O |
| ATOM | 58 | CB | ILE | A | 329 | 25.163 | 46.557 | 54.527 | 1.00 | 29.19 | C |
| ATOM | 59 | CG1 | ILE | A | 329 | 24.320 | 46.574 | 53.249 | 1.00 | 29.38 | C |
| ATOM | 60 | CG2 | ILE | A | 329 | 24.403 | 47.188 | 55.704 | 1.00 | 29.20 | C |
| ATOM | 61 | CD1 | ILE | A | 329 | 22.992 | 45.930 | 53.392 | 1.00 | 31.12 | C |
| ATOM | 62 | N | PHE | A | 330 | 28.148 | 46.243 | 55.691 | 1.00 | 28.67 | N |
| ATOM | 63 | CA | PHE | A | 330 | 29.116 | 46.188 | 56.774 | 1.00 | 28.49 | C |
| ATOM | 64 | C | PHE | A | 330 | 30.485 | 45.859 | 56.198 | 1.00 | 28.56 | C |
| ATOM | 65 | O | PHE | A | 330 | 30.736 | 44.737 | 55.768 | 1.00 | 28.31 | O |
| ATOM | 66 | CB | PHE | A | 330 | 28.686 | 45.175 | 57.843 | 1.00 | 28.25 | C |
| ATOM | 67 | CG | PHE | A | 330 | 27.335 | 45.451 | 58.434 | 1.00 | 27.52 | C |
| ATOM | 68 | CD1 | PHE | A | 330 | 27.167 | 46.449 | 59.384 | 1.00 | 27.43 | C |
| ATOM | 69 | CD2 | PHE | A | 330 | 26.229 | 44.712 | 58.040 | 1.00 | 26.97 | C |
| ATOM | 70 | CE1 | PHE | A | 330 | 25.910 | 46.706 | 59.933 | 1.00 | 26.69 | C |
| ATOM | 71 | CE2 | PHE | A | 330 | 24.978 | 44.962 | 58.584 | 1.00 | 26.68 | C |
| ATOM | 72 | CZ | PHE | A | 330 | 24.820 | 45.964 | 59.532 | 1.00 | 26.63 | C |
| ATOM | 73 | N | GLU | A | 331 | 31.352 | 46.869 | 56.178 | 1.00 | 29.24 | N |
| ATOM | 74 | CA | GLU | A | 331 | 32.663 | 46.789 | 55.536 | 1.00 | 29.53 | C |
| ATOM | 75 | C | GLU | A | 331 | 33.765 | 46.450 | 56.531 | 1.00 | 29.79 | C |
| ATOM | 76 | O | GLU | A | 331 | 34.145 | 47.264 | 57.373 | 1.00 | 29.56 | O |
| ATOM | 77 | CB | GLU | A | 331 | 32.978 | 48.099 | 54.806 | 1.00 | 29.60 | C |
| ATOM | 78 | CG | GLU | A | 331 | 32.340 | 48.199 | 53.422 | 1.00 | 30.13 | C |
| ATOM | 79 | CD | GLU | A | 331 | 32.114 | 49.631 | 52.963 | 1.00 | 30.75 | C |
| ATOM | 80 | OE1 | GLU | A | 331 | 31.891 | 50.513 | 53.825 | 1.00 | 30.82 | O |
| ATOM | 81 | OE2 | GLU | A | 331 | 32.138 | 49.871 | 51.732 | 1.00 | 31.12 | O |
| ATOM | 82 | N | SER | A | 332 | 34.260 | 45.224 | 56.429 | 1.00 | 30.39 | N |
| ATOM | 83 | CA | SER | A | 332 | 35.356 | 44.748 | 57.250 | 1.00 | 31.12 | C |
| ATOM | 84 | C | SER | A | 332 | 36.671 | 45.340 | 56.765 | 1.00 | 31.94 | C |
| ATOM | 85 | O | SER | A | 332 | 36.772 | 45.795 | 55.624 | 1.00 | 31.94 | O |
| ATOM | 86 | CB | SER | A | 332 | 35.439 | 43.238 | 57.128 | 1.00 | 31.01 | C |
| ATOM | 87 | OG | SER | A | 332 | 35.598 | 42.890 | 55.764 | 1.00 | 30.34 | O |
| ATOM | 88 | N | GLY | A | 333 | 37.683 | 45.299 | 57.626 | 1.00 | 32.89 | N |
| ATOM | 89 | CA | GLY | A | 333 | 39.024 | 45.747 | 57.270 | 1.00 | 34.20 | C |
| ATOM | 90 | C | GLY | A | 333 | 39.639 | 44.943 | 56.139 | 1.00 | 35.07 | C |
| ATOM | 91 | O | GLY | A | 333 | 38.975 | 44.135 | 55.489 | 1.00 | 35.10 | O |
| ATOM | 92 | N | ARG | A | 334 | 40.926 | 45.155 | 55.910 | 1.00 | 36.11 | N |
| ATOM | 93 | CA | ARG | A | 334 | 41.588 | 44.555 | 54.763 | 1.00 | 37.10 | C |
| ATOM | 94 | C | ARG | A | 334 | 42.965 | 44.028 | 55.157 | 1.00 | 37.48 | C |
| ATOM | 95 | O | ARG | A | 334 | 43.701 | 44.697 | 55.883 | 1.00 | 37.79 | O |
| ATOM | 96 | CB | ARG | A | 334 | 41.694 | 45.591 | 53.646 | 1.00 | 37.10 | C |
| ATOM | 97 | CG | ARG | A | 334 | 41.622 | 45.022 | 52.252 | 1.00 | 38.09 | C |
| ATOM | 98 | CD | ARG | A | 334 | 41.571 | 46.140 | 51.223 | 1.00 | 40.06 | C |
| ATOM | 99 | NE | ARG | A | 334 | 40.205 | 46.614 | 50.985 | 1.00 | 41.41 | N |
| ATOM | 100 | CZ | ARG | A | 334 | 39.393 | 46.132 | 50.044 | 1.00 | 42.25 | C |
| ATOM | 101 | NH1 | ARG | A | 334 | 39.799 | 45.146 | 49.250 | 1.00 | 42.54 | N |
| ATOM | 102 | NH2 | ARG | A | 334 | 38.169 | 46.629 | 49.898 | 1.00 | 42.42 | N |
| ATOM | 103 | N | ASN | A | 335 | 43.287 | 42.816 | 54.702 | 1.00 | 38.00 | N |
| ATOM | 104 | CA | ASN | A | 335 | 44.598 | 42.185 | 54.940 | 1.00 | 38.53 | C |
| ATOM | 105 | C | ASN | A | 335 | 44.981 | 42.033 | 56.420 | 1.00 | 38.73 | C |
| ATOM | 106 | O | ASN | A | 335 | 46.141 | 42.228 | 56.797 | 1.00 | 38.87 | O |
| ATOM | 107 | CB | ASN | A | 335 | 45.703 | 42.923 | 54.169 | 1.00 | 38.55 | C |
| ATOM | 108 | CG | ASN | A | 335 | 45.397 | 43.047 | 52.692 | 1.00 | 39.10 | C |
| ATOM | 109 | OD1 | ASN | A | 335 | 45.242 | 42.041 | 51.992 | 1.00 | 39.31 | O |
| ATOM | 110 | ND2 | ASN | A | 335 | 45.303 | 44.286 | 52.206 | 1.00 | 39.00 | N |
| ATOM | 111 | N | GLY | A | 336 | 43.998 | 41.680 | 57.244 | 1.00 | 38.91 | N |
| ATOM | 112 | CA | GLY | A | 336 | 44.207 | 41.458 | 58.671 | 1.00 | 38.98 | C |
| ATOM | 113 | C | GLY | A | 336 | 44.160 | 42.735 | 59.486 | 1.00 | 39.17 | C |
| ATOM | 114 | O | GLY | A | 336 | 44.228 | 42.692 | 60.715 | 1.00 | 39.17 | O |
| ATOM | 115 | N | LYS | A | 337 | 44.022 | 43.867 | 58.799 | 1.00 | 39.32 | N |
| ATOM | 116 | CA | LYS | A | 337 | 44.107 | 45.185 | 59.426 | 1.00 | 39.69 | C |
| ATOM | 117 | C | LYS | A | 337 | 42.714 | 45.775 | 59.688 | 1.00 | 39.64 | C |
| ATOM | 118 | O | LYS | A | 337 | 41.761 | 45.425 | 58.989 | 1.00 | 39.89 | O |
| ATOM | 119 | CB | LYS | A | 337 | 44.952 | 46.125 | 58.551 | 1.00 | 39.94 | C |
| ATOM | 120 | CG | LYS | A | 337 | 46.244 | 45.483 | 58.017 | 1.00 | 40.83 | C |
| ATOM | 121 | CD | LYS | A | 337 | 47.322 | 46.512 | 57.697 | 1.00 | 42.53 | C |
| ATOM | 122 | CE | LYS | A | 337 | 48.675 | 45.833 | 57.486 | 1.00 | 43.32 | C |
| ATOM | 123 | NZ | LYS | A | 337 | 49.826 | 46.788 | 57.564 | 1.00 | 43.60 | N |
| ATOM | 124 | N | PRO | A | 338 | 42.585 | 46.669 | 60.695 | 1.00 | 39.49 | N |
| ATOM | 125 | CA | PRO | A | 338 | 41.265 | 47.234 | 60.990 | 1.00 | 39.39 | C |
| ATOM | 126 | C | PRO | A | 338 | 40.805 | 48.174 | 59.890 | 1.00 | 39.40 | C |
| ATOM | 127 | O | PRO | A | 338 | 41.629 | 48.672 | 59.123 | 1.00 | 39.58 | O |
| ATOM | 128 | CB | PRO | A | 338 | 41.493 | 48.028 | 62.288 | 1.00 | 39.41 | C |
| ATOM | 129 | CG | PRO | A | 338 | 42.826 | 47.590 | 62.809 | 1.00 | 39.36 | C |
| ATOM | 130 | CD | PRO | A | 338 | 43.616 | 47.206 | 61.600 | 1.00 | 39.38 | C |
| ATOM | 131 | N | ASN | A | 339 | 39.500 | 48.407 | 59.808 | 1.00 | 39.36 | N |
| ATOM | 132 | CA | ASN | A | 339 | 38.961 | 49.373 | 58.862 | 1.00 | 39.08 | C |
| ATOM | 133 | C | ASN | A | 339 | 39.037 | 50.792 | 59.424 | 1.00 | 39.21 | C |
| ATOM | 134 | O | ASN | A | 339 | 39.694 | 51.028 | 60.440 | 1.00 | 39.20 | O |
| ATOM | 135 | CB | ASN | A | 339 | 37.531 | 48.993 | 58.425 | 1.00 | 39.15 | C |
| ATOM | 136 | CG | ASN | A | 339 | 36.486 | 49.158 | 59.536 | 1.00 | 38.50 | C |
| ATOM | 137 | OD1 | ASN | A | 339 | 36.751 | 49.736 | 60.592 | 1.00 | 38.54 | O |
| ATOM | 138 | ND2 | ASN | A | 339 | 35.284 | 48.651 | 59.283 | 1.00 | 36.87 | N |
| ATOM | 139 | N | LYS | A | 340 | 38.347 | 51.723 | 58.769 | 1.00 | 39.27 | N |
| ATOM | 140 | CA | LYS | A | 340 | 38.376 | 53.142 | 59.128 | 1.00 | 39.37 | C |
| ATOM | 141 | C | LYS | A | 340 | 37.870 | 53.437 | 60.539 | 1.00 | 39.02 | C |
| ATOM | 142 | O | LYS | A | 340 | 38.248 | 54.448 | 61.130 | 1.00 | 39.12 | O |
| ATOM | 143 | CB | LYS | A | 340 | 37.583 | 53.950 | 58.097 | 1.00 | 39.58 | C |
| ATOM | 144 | CG | LYS | A | 340 | 38.269 | 53.978 | 56.750 | 1.00 | 41.13 | C |
| ATOM | 145 | CD | LYS | A | 340 | 37.355 | 53.536 | 55.610 | 1.00 | 42.86 | C |
| ATOM | 146 | CE | LYS | A | 340 | 38.194 | 52.959 | 54.475 | 1.00 | 43.62 | C |
| ATOM | 147 | NZ | LYS | A | 340 | 37.589 | 53.198 | 53.138 | 1.00 | 45.04 | N |
| ATOM | 148 | N | ASP | A | 341 | 37.026 | 52.553 | 61.068 | 1.00 | 38.51 | N |
| ATOM | 149 | CA | ASP | A | 341 | 36.457 | 52.715 | 62.406 | 1.00 | 38.06 | C |
| ATOM | 150 | C | ASP | A | 341 | 37.210 | 51.950 | 63.490 | 1.00 | 37.55 | C |
| ATOM | 151 | O | ASP | A | 341 | 36.918 | 52.109 | 64.675 | 1.00 | 37.75 | O |
| ATOM | 152 | CB | ASP | A | 341 | 34.977 | 52.315 | 62.409 | 1.00 | 38.13 | C |
| ATOM | 153 | CG | ASP | A | 341 | 34.138 | 53.167 | 61.466 | 1.00 | 38.60 | C |
| ATOM | 154 | OD1 | ASP | A | 341 | 34.677 | 54.131 | 60.876 | 1.00 | 39.05 | O |
| ATOM | 155 | OD2 | ASP | A | 341 | 32.937 | 52.873 | 61.310 | 1.00 | 38.41 | O |
| ATOM | 156 | N | GLY | A | 342 | 38.176 | 51.128 | 63.082 | 1.00 | 37.00 | N |
| ATOM | 157 | CA | GLY | A | 342 | 38.928 | 50.275 | 64.014 | 1.00 | 36.18 | C |
| ATOM | 158 | C | GLY | A | 342 | 38.399 | 48.850 | 64.104 | 1.00 | 35.68 | C |
| ATOM | 159 | O | GLY | A | 342 | 38.748 | 48.101 | 65.021 | 1.00 | 35.59 | O |
| ATOM | 160 | N | ILE | A | 343 | 37.567 | 48.474 | 63.137 | 1.00 | 35.12 | N |
| ATOM | 161 | CA | ILE | A | 343 | 36.891 | 47.181 | 63.136 | 1.00 | 34.44 | C |
| ATOM | 162 | C | ILE | A | 343 | 37.482 | 46.258 | 62.072 | 1.00 | 33.85 | C |
| ATOM | 163 | O | ILE | A | 343 | 37.526 | 46.609 | 60.893 | 1.00 | 33.74 | O |
| ATOM | 164 | CB | ILE | A | 343 | 35.358 | 47.362 | 62.936 | 1.00 | 34.51 | C |
| ATOM | 165 | CG1 | ILE | A | 343 | 34.752 | 48.069 | 64.155 | 1.00 | 34.45 | C |
| ATOM | 166 | CG2 | ILE | A | 343 | 34.663 | 46.019 | 62.705 | 1.00 | 34.60 | C |
| ATOM | 167 | CD1 | ILE | A | 343 | 33.600 | 48.974 | 63.828 | 1.00 | 34.27 | C |
| ATOM | 168 | N | LYS | A | 344 | 37.932 | 45.083 | 62.502 | 1.00 | 33.13 | N |
| ATOM | 169 | CA | LYS | A | 344 | 38.519 | 44.095 | 61.597 | 1.00 | 32.92 | C |
| ATOM | 170 | C | LYS | A | 344 | 37.474 | 43.267 | 60.859 | 1.00 | 32.63 | C |
| ATOM | 171 | O | LYS | A | 344 | 37.668 | 42.919 | 59.698 | 1.00 | 32.83 | O |
| ATOM | 172 | CB | LYS | A | 344 | 39.473 | 43.146 | 62.341 | 1.00 | 32.80 | C |
| ATOM | 173 | CG | LYS | A | 344 | 40.778 | 43.781 | 62.767 | 1.00 | 32.92 | C |
| ATOM | 174 | CD | LYS | A | 344 | 41.797 | 42.749 | 63.204 | 1.00 | 32.67 | C |
| ATOM | 175 | CE | LYS | A | 344 | 42.870 | 43.415 | 64.045 | 1.00 | 33.04 | C |
| ATOM | 176 | NZ | LYS | A | 344 | 44.179 | 42.735 | 63.903 | 1.00 | 33.16 | N |
| ATOM | 177 | N | SER | A | 345 | 36.374 | 42.944 | 61.531 | 1.00 | 32.16 | N |
| ATOM | 178 | CA | SER | A | 345 | 35.434 | 41.964 | 60.998 | 1.00 | 31.66 | C |
| ATOM | 179 | C | SER | A | 345 | 33.976 | 42.293 | 61.267 | 1.00 | 30.99 | C |
| ATOM | 180 | O | SER | A | 345 | 33.632 | 42.812 | 62.323 | 1.00 | 31.06 | O |
| ATOM | 181 | CB | SER | A | 345 | 35.751 | 40.579 | 61.569 | 1.00 | 31.68 | C |
| ATOM | 182 | OG | SER | A | 345 | 34.727 | 39.647 | 61.259 | 1.00 | 32.06 | O |
| ATOM | 183 | N | TYR | A | 346 | 33.138 | 41.993 | 60.279 | 1.00 | 30.39 | N |
| ATOM | 184 | CA | TYR | A | 346 | 31.691 | 41.878 | 60.458 | 1.00 | 29.86 | C |
| ATOM | 185 | C | TYR | A | 346 | 31.294 | 40.471 | 60.073 | 1.00 | 29.67 | C |
| ATOM | 186 | O | TYR | A | 346 | 31.998 | 39.809 | 59.300 | 1.00 | 29.73 | O |
| ATOM | 187 | CB | TYR | A | 346 | 30.939 | 42.893 | 59.607 | 1.00 | 29.70 | C |
| ATOM | 188 | CG | TYR | A | 346 | 30.993 | 44.280 | 60.178 | 1.00 | 29.05 | C |
| ATOM | 189 | CD1 | TYR | A | 346 | 30.108 | 44.668 | 61.177 | 1.00 | 28.28 | C |
| ATOM | 190 | CD2 | TYR | A | 346 | 31.936 | 45.203 | 59.728 | 1.00 | 28.45 | C |
| ATOM | 191 | CE1 | TYR | A | 346 | 30.147 | 45.939 | 61.713 | 1.00 | 28.72 | C |
| ATOM | 192 | CE2 | TYR | A | 346 | 31.985 | 46.485 | 60.259 | 1.00 | 28.98 | C |
| ATOM | 193 | CZ | TYR | A | 346 | 31.086 | 46.844 | 61.256 | 1.00 | 28.89 | C |
| ATOM | 194 | OH | TYR | A | 346 | 31.118 | 48.103 | 61.797 | 1.00 | 28.99 | O |
| ATOM | 195 | N | ARG | A | 347 | 30.173 | 39.998 | 60.601 | 1.00 | 29.19 | N |
| ATOM | 196 | CA | ARG | A | 347 | 29.931 | 38.574 | 60.515 | 1.00 | 28.81 | C |
| ATOM | 197 | C | ARG | A | 347 | 28.497 | 38.119 | 60.244 | 1.00 | 28.49 | C |
| ATOM | 198 | O | ARG | A | 347 | 28.126 | 37.934 | 59.091 | 1.00 | 29.41 | O |
| ATOM | 199 | CB | ARG | A | 347 | 30.533 | 37.884 | 61.736 | 1.00 | 28.82 | C |
| ATOM | 200 | CG | ARG | A | 347 | 30.863 | 36.432 | 61.525 | 1.00 | 29.29 | C |
| ATOM | 201 | CD | ARG | A | 347 | 31.917 | 36.202 | 60.452 | 1.00 | 28.12 | C |
| ATOM | 202 | NE | ARG | A | 347 | 32.466 | 34.860 | 60.581 | 1.00 | 26.79 | N |
| ATOM | 203 | CZ | ARG | A | 347 | 31.842 | 33.735 | 60.239 | 1.00 | 25.34 | C |
| ATOM | 204 | NH1 | ARG | A | 347 | 30.620 | 33.755 | 59.726 | 1.00 | 24.11 | N |
| ATOM | 205 | NH2 | ARG | A | 347 | 32.456 | 32.578 | 60.417 | 1.00 | 25.06 | N |
| ATOM | 206 | N | ILE | A | 348 | 27.694 | 37.936 | 61.281 | 1.00 | 27.51 | N |
| ATOM | 207 | CA | ILE | A | 348 | 26.475 | 37.125 | 61.157 | 1.00 | 26.38 | C |
| ATOM | 208 | C | ILE | A | 348 | 25.240 | 37.916 | 60.699 | 1.00 | 25.66 | C |
| ATOM | 209 | O | ILE | A | 348 | 24.578 | 38.548 | 61.513 | 1.00 | 25.60 | O |
| ATOM | 210 | CB | ILE | A | 348 | 26.174 | 36.395 | 62.488 | 1.00 | 26.24 | C |
| ATOM | 211 | CG1 | ILE | A | 348 | 27.458 | 35.795 | 63.082 | 1.00 | 25.88 | C |
| ATOM | 212 | CG2 | ILE | A | 348 | 25.093 | 35.358 | 62.293 | 1.00 | 26.28 | C |
| ATOM | 213 | CD1 | ILE | A | 348 | 28.075 | 34.649 | 62.278 | 1.00 | 25.29 | C |
| ATOM | 214 | N | PRO | A | 349 | 24.902 | 37.843 | 59.401 | 1.00 | 25.14 | N |
| ATOM | 215 | CA | PRO | A | 349 | 23.852 | 38.696 | 58.857 | 1.00 | 24.90 | C |
| ATOM | 216 | C | PRO | A | 349 | 22.427 | 38.279 | 59.235 | 1.00 | 24.60 | C |
| ATOM | 217 | O | PRO | A | 349 | 22.101 | 37.088 | 59.273 | 1.00 | 24.31 | O |
| ATOM | 218 | CB | PRO | A | 349 | 24.048 | 38.564 | 57.343 | 1.00 | 24.88 | C |
| ATOM | 219 | CG | PRO | A | 349 | 24.622 | 37.222 | 57.153 | 1.00 | 24.93 | C |
| ATOM | 220 | CD | PRO | A | 349 | 25.458 | 36.942 | 58.373 | 1.00 | 25.22 | C |
| ATOM | 221 | N | ALA | A | 350 | 21.600 | 39.281 | 59.514 | 1.00 | 24.17 | N |
| ATOM | 222 | CA | ALA | A | 350 | 20.160 | 39.106 | 59.617 | 1.00 | 23.78 | C |
| ATOM | 223 | C | ALA | A | 350 | 19.497 | 40.271 | 58.895 | 1.00 | 23.54 | C |
| ATOM | 224 | O | ALA | A | 350 | 19.963 | 41.412 | 58.978 | 1.00 | 23.18 | O |
| ATOM | 225 | CB | ALA | A | 350 | 19.721 | 39.049 | 61.072 | 1.00 | 23.76 | C |
| ATOM | 226 | N | LEU | A | 351 | 18.418 | 39.969 | 58.179 | 1.00 | 23.31 | N |
| ATOM | 227 | CA | LEU | A | 351 | 17.697 | 40.965 | 57.405 | 1.00 | 23.13 | C |
| ATOM | 228 | C | LEU | A | 351 | 16.202 | 40.907 | 57.702 | 1.00 | 23.39 | C |
| ATOM | 229 | O | LEU | A | 351 | 15.585 | 39.836 | 57.647 | 1.00 | 22.95 | O |
| ATOM | 230 | CB | LEU | A | 351 | 17.956 | 40.755 | 55.908 | 1.00 | 23.17 | C |
| ATOM | 231 | CG | LEU | A | 351 | 17.296 | 41.681 | 54.879 | 1.00 | 22.81 | C |
| ATOM | 232 | CD1 | LEU | A | 351 | 17.969 | 43.055 | 54.855 | 1.00 | 22.43 | C |
| ATOM | 233 | CD2 | LEU | A | 351 | 17.340 | 41.032 | 53.497 | 1.00 | 22.62 | C |
| ATOM | 234 | N | LEU | A | 352 | 15.627 | 42.069 | 58.000 | 1.00 | 23.74 | N |
| ATOM | 235 | CA | LEU | A | 352 | 14.204 | 42.164 | 58.315 | 1.00 | 24.04 | C |
| ATOM | 236 | C | LEU | A | 352 | 13.492 | 43.266 | 57.531 | 1.00 | 24.38 | C |
| ATOM | 237 | O | LEU | A | 352 | 13.972 | 44.394 | 57.444 | 1.00 | 24.28 | O |
| ATOM | 238 | CB | LEU | A | 352 | 14.009 | 42.376 | 59.820 | 1.00 | 24.03 | C |
| ATOM | 239 | CG | LEU | A | 352 | 12.602 | 42.258 | 60.418 | 1.00 | 23.64 | C |
| ATOM | 240 | CD1 | LEU | A | 352 | 11.988 | 40.900 | 60.139 | 1.00 | 22.93 | C |
| ATOM | 241 | CD2 | LEU | A | 352 | 12.663 | 42.521 | 61.916 | 1.00 | 23.76 | C |
| ATOM | 242 | N | LYS | A | 353 | 12.343 | 42.917 | 56.962 | 1.00 | 24.93 | N |
| ATOM | 243 | CA | LYS | A | 353 | 11.482 | 43.878 | 56.289 | 1.00 | 25.51 | C |
| ATOM | 244 | C | LYS | A | 353 | 10.321 | 44.207 | 57.223 | 1.00 | 25.92 | C |
| ATOM | 245 | O | LYS | A | 353 | 9.479 | 43.349 | 57.505 | 1.00 | 26.10 | O |
| ATOM | 246 | CB | LYS | A | 353 | 10.992 | 43.310 | 54.951 | 1.00 | 25.43 | C |
| ATOM | 247 | CG | LYS | A | 353 | 9.844 | 44.078 | 54.290 | 1.00 | 26.24 | C |
| ATOM | 248 | CD | LYS | A | 353 | 10.319 | 45.283 | 53.506 | 1.00 | 26.71 | C |
| ATOM | 249 | CE | LYS | A | 353 | 9.299 | 45.663 | 52.445 | 1.00 | 27.33 | C |
| ATOM | 250 | NZ | LYS | A | 353 | 9.793 | 46.747 | 51.545 | 1.00 | 28.34 | N |
| ATOM | 251 | N | THR | A | 354 | 10.290 | 45.446 | 57.707 | 1.00 | 26.34 | N |
| ATOM | 252 | CA | THR | A | 354 | 9.312 | 45.856 | 58.713 | 1.00 | 26.83 | C |
| ATOM | 253 | C | THR | A | 354 | 7.991 | 46.286 | 58.094 | 1.00 | 27.05 | C |
| ATOM | 254 | O | THR | A | 354 | 7.927 | 46.640 | 56.913 | 1.00 | 27.14 | O |
| ATOM | 255 | CB | THR | A | 354 | 9.835 | 47.017 | 59.594 | 1.00 | 27.06 | C |
| ATOM | 256 | OG1 | THR | A | 354 | 9.667 | 48.263 | 58.904 | 1.00 | 27.15 | O |
| ATOM | 257 | CG2 | THR | A | 354 | 11.307 | 46.818 | 59.956 | 1.00 | 26.85 | C |
| ATOM | 258 | N | ASP | A | 355 | 6.943 | 46.276 | 58.913 | 1.00 | 27.35 | N |
| ATOM | 259 | CA | ASP | A | 355 | 5.617 | 46.725 | 58.496 | 1.00 | 27.71 | C |
| ATOM | 260 | C | ASP | A | 355 | 5.641 | 48.118 | 57.851 | 1.00 | 27.68 | C |
| ATOM | 261 | O | ASP | A | 355 | 4.872 | 48.382 | 56.932 | 1.00 | 27.74 | O |
| ATOM | 262 | CB | ASP | A | 355 | 4.660 | 46.716 | 59.685 | 1.00 | 27.94 | C |
| ATOM | 263 | CG | ASP | A | 355 | 4.905 | 47.867 | 60.637 | 1.00 | 29.40 | C |
| ATOM | 264 | OD1 | ASP | A | 355 | 6.036 | 47.986 | 61.160 | 1.00 | 31.07 | O |
| ATOM | 265 | OD2 | ASP | A | 355 | 3.966 | 48.661 | 60.859 | 1.00 | 31.22 | O |
| ATOM | 266 | N | LYS | A | 356 | 6.536 | 48.990 | 58.323 | 1.00 | 27.51 | N |
| ATOM | 267 | CA | LYS | A | 356 | 6.687 | 50.340 | 57.774 | 1.00 | 27.35 | C |
| ATOM | 268 | C | LYS | A | 356 | 7.400 | 50.361 | 56.417 | 1.00 | 27.10 | C |
| ATOM | 269 | O | LYS | A | 356 | 7.531 | 51.422 | 55.809 | 1.00 | 27.21 | O |
| ATOM | 270 | CB | LYS | A | 356 | 7.418 | 51.262 | 58.768 | 1.00 | 27.53 | C |
| ATOM | 271 | CG | LYS | A | 356 | 6.581 | 51.769 | 59.965 | 1.00 | 28.45 | C |
| ATOM | 272 | CD | LYS | A | 356 | 5.491 | 52.772 | 59.537 | 1.00 | 30.42 | C |
| ATOM | 273 | CE | LYS | A | 356 | 5.221 | 53.838 | 60.618 | 1.00 | 32.33 | C |
| ATOM | 274 | NZ | LYS | A | 356 | 4.313 | 53.411 | 61.736 | 1.00 | 31.55 | N |
| ATOM | 275 | N | GLY | A | 357 | 7.859 | 49.199 | 55.949 | 1.00 | 26.67 | N |
| ATOM | 276 | CA | GLY | A | 357 | 8.560 | 49.094 | 54.665 | 1.00 | 25.88 | C |
| ATOM | 277 | C | GLY | A | 357 | 10.072 | 49.196 | 54.787 | 1.00 | 25.51 | C |
| ATOM | 278 | O | GLY | A | 357 | 10.798 | 49.009 | 53.810 | 1.00 | 25.31 | O |
| ATOM | 279 | N | THR | A | 358 | 10.538 | 49.486 | 55.997 | 1.00 | 25.09 | N |
| ATOM | 280 | CA | THR | A | 358 | 11.956 | 49.614 | 56.308 | 1.00 | 24.94 | C |
| ATOM | 281 | C | THR | A | 358 | 12.716 | 48.281 | 56.214 | 1.00 | 24.92 | C |
| ATOM | 282 | O | THR | A | 358 | 12.170 | 47.220 | 56.524 | 1.00 | 24.85 | O |
| ATOM | 283 | CB | THR | A | 358 | 12.101 | 50.183 | 57.721 | 1.00 | 25.01 | C |
| ATOM | 284 | OG1 | THR | A | 358 | 11.295 | 51.364 | 57.833 | 1.00 | 25.55 | O |
| ATOM | 285 | CG2 | THR | A | 358 | 13.553 | 50.499 | 58.067 | 1.00 | 24.51 | C |
| ATOM | 286 | N | LEU | A | 359 | 13.969 | 48.342 | 55.772 | 1.00 | 24.81 | N |
| ATOM | 287 | CA | LEU | A | 359 | 14.850 | 47.181 | 55.832 | 1.00 | 25.03 | C |
| ATOM | 288 | C | LEU | A | 359 | 15.843 | 47.343 | 56.972 | 1.00 | 25.15 | C |
| ATOM | 289 | O | LEU | A | 359 | 16.519 | 48.375 | 57.086 | 1.00 | 25.24 | O |
| ATOM | 290 | CB | LEU | A | 359 | 15.587 | 46.957 | 54.509 | 1.00 | 25.21 | C |
| ATOM | 291 | CG | LEU | A | 359 | 14.781 | 46.468 | 53.299 | 1.00 | 25.20 | C |
| ATOM | 292 | CD1 | LEU | A | 359 | 15.661 | 46.440 | 52.055 | 1.00 | 25.07 | C |
| ATOM | 293 | CD2 | LEU | A | 359 | 14.151 | 45.094 | 53.553 | 1.00 | 25.61 | C |
| ATOM | 294 | N | ILE | A | 360 | 15.907 | 46.325 | 57.825 | 1.00 | 24.95 | N |
| ATOM | 295 | CA | ILE | A | 360 | 16.827 | 46.310 | 58.954 | 1.00 | 24.62 | C |
| ATOM | 296 | C | ILE | A | 360 | 17.867 | 45.226 | 58.729 | 1.00 | 24.61 | C |
| ATOM | 297 | O | ILE | A | 360 | 17.532 | 44.063 | 58.479 | 1.00 | 24.37 | O |
| ATOM | 298 | CB | ILE | A | 360 | 16.089 | 46.100 | 60.301 | 1.00 | 24.63 | C |
| ATOM | 299 | CG1 | ILE | A | 360 | 15.068 | 47.225 | 60.514 | 1.00 | 24.76 | C |
| ATOM | 300 | CG2 | ILE | A | 360 | 17.078 | 46.056 | 61.462 | 1.00 | 24.01 | C |
| ATOM | 301 | CD1 | ILE | A | 360 | 14.451 | 47.273 | 61.894 | 1.00 | 24.77 | C |
| ATOM | 302 | N | ALA | A | 361 | 19.130 | 45.627 | 58.808 | 1.00 | 24.60 | N |
| ATOM | 303 | CA | ALA | A | 361 | 20.238 | 44.723 | 58.591 | 1.00 | 24.94 | C |
| ATOM | 304 | C | ALA | A | 361 | 21.066 | 44.586 | 59.861 | 1.00 | 25.18 | C |
| ATOM | 305 | O | ALA | A | 361 | 21.652 | 45.561 | 60.344 | 1.00 | 25.18 | O |
| ATOM | 306 | CB | ALA | A | 361 | 21.102 | 45.211 | 57.421 | 1.00 | 24.77 | C |
| ATOM | 307 | N | GLY | A | 362 | 21.109 | 43.370 | 60.395 | 1.00 | 25.45 | N |
| ATOM | 308 | CA | GLY | A | 362 | 21.837 | 43.090 | 61.629 | 1.00 | 25.87 | C |
| ATOM | 309 | C | GLY | A | 362 | 23.137 | 42.354 | 61.380 | 1.00 | 26.22 | C |
| ATOM | 310 | O | GLY | A | 362 | 23.284 | 41.679 | 60.359 | 1.00 | 26.25 | O |
| ATOM | 311 | N | ALA | A | 363 | 24.075 | 42.479 | 62.319 | 1.00 | 26.41 | N |
| ATOM | 312 | CA | ALA | A | 363 | 25.381 | 41.818 | 62.222 | 1.00 | 26.59 | C |
| ATOM | 313 | C | ALA | A | 363 | 26.126 | 41.768 | 63.555 | 1.00 | 26.72 | C |
| ATOM | 314 | O | ALA | A | 363 | 25.861 | 42.559 | 64.461 | 1.00 | 26.55 | O |
| ATOM | 315 | CB | ALA | A | 363 | 26.249 | 42.524 | 61.174 | 1.00 | 26.57 | C |
| ATOM | 316 | N | ASP | A | 364 | 27.056 | 40.824 | 63.659 | 1.00 | 27.32 | N |
| ATOM | 317 | CA | ASP | A | 364 | 28.113 | 40.872 | 64.670 | 1.00 | 27.96 | C |
| ATOM | 318 | C | ASP | A | 364 | 29.148 | 41.914 | 64.252 | 1.00 | 28.25 | C |
| ATOM | 319 | O | ASP | A | 364 | 29.701 | 41.834 | 63.153 | 1.00 | 28.09 | O |
| ATOM | 320 | CB | ASP | A | 364 | 28.823 | 39.523 | 64.769 | 1.00 | 28.03 | C |
| ATOM | 321 | CG | ASP | A | 364 | 28.051 | 38.499 | 65.580 | 1.00 | 28.42 | C |
| ATOM | 322 | OD1 | ASP | A | 364 | 26.808 | 38.599 | 65.703 | 1.00 | 28.94 | O |
| ATOM | 323 | OD2 | ASP | A | 364 | 28.711 | 37.568 | 66.085 | 1.00 | 28.84 | O |
| ATOM | 324 | N | GLU | A | 365 | 29.402 | 42.889 | 65.116 | 1.00 | 28.82 | N |
| ATOM | 325 | CA | GLU | A | 365 | 30.506 | 43.825 | 64.902 | 1.00 | 29.60 | C |
| ATOM | 326 | C | GLU | A | 365 | 31.732 | 43.329 | 65.671 | 1.00 | 29.82 | C |
| ATOM | 327 | O | GLU | A | 365 | 31.867 | 43.586 | 66.872 | 1.00 | 29.92 | O |
| ATOM | 328 | CB | GLU | A | 365 | 30.113 | 45.237 | 65.343 | 1.00 | 29.43 | C |
| ATOM | 329 | CG | GLU | A | 365 | 31.176 | 46.299 | 65.088 | 1.00 | 30.08 | C |
| ATOM | 330 | CD | GLU | A | 365 | 30.750 | 47.681 | 65.553 | 1.00 | 30.47 | C |
| ATOM | 331 | OE1 | GLU | A | 365 | 30.573 | 47.879 | 66.801 | 1.00 | 31.38 | O |
| ATOM | 332 | OE2 | GLU | A | 365 | 30.597 | 48.577 | 64.661 | 1.00 | 32.02 | O |
| ATOM | 333 | N | ARG | A | 366 | 32.609 | 42.604 | 64.979 | 1.00 | 30.14 | N |
| ATOM | 334 | CA | ARG | A | 366 | 33.786 | 42.000 | 65.610 | 1.00 | 30.64 | C |
| ATOM | 335 | C | ARG | A | 366 | 35.027 | 42.888 | 65.433 | 1.00 | 31.44 | C |
| ATOM | 336 | O | ARG | A | 366 | 35.735 | 42.794 | 64.422 | 1.00 | 31.37 | O |
| ATOM | 337 | CB | ARG | A | 366 | 34.010 | 40.577 | 65.077 | 1.00 | 30.44 | C |
| ATOM | 338 | CG | ARG | A | 366 | 32.838 | 39.628 | 65.330 | 1.00 | 30.23 | C |
| ATOM | 339 | CD | ARG | A | 366 | 33.048 | 38.251 | 64.723 | 1.00 | 30.14 | C |
| ATOM | 340 | NE | ARG | A | 366 | 31.859 | 37.412 | 64.884 | 1.00 | 30.01 | N |
| ATOM | 341 | CZ | ARG | A | 366 | 31.818 | 36.092 | 64.690 | 1.00 | 29.73 | C |
| ATOM | 342 | NH1 | ARG | A | 366 | 32.904 | 35.422 | 64.317 | 1.00 | 29.14 | N |
| ATOM | 343 | NH2 | ARG | A | 366 | 30.678 | 35.437 | 64.867 | 1.00 | 28.73 | N |
| ATOM | 344 | N | ARG | A | 367 | 35.274 | 43.743 | 66.432 | 1.00 | 32.27 | N |
| ATOM | 345 | CA | ARG | A | 367 | 36.270 | 44.828 | 66.350 | 1.00 | 33.32 | C |
| ATOM | 346 | C | ARG | A | 367 | 37.701 | 44.372 | 66.026 | 1.00 | 33.71 | C |
| ATOM | 347 | O | ARG | A | 367 | 38.281 | 44.799 | 65.029 | 1.00 | 33.82 | O |
| ATOM | 348 | CB | ARG | A | 367 | 36.244 | 45.687 | 67.626 | 1.00 | 33.36 | C |
| ATOM | 349 | CG | ARG | A | 367 | 37.319 | 46.769 | 67.688 | 1.00 | 33.75 | C |
| ATOM | 350 | CD | ARG | A | 367 | 37.065 | 47.780 | 68.801 | 1.00 | 34.04 | C |
| ATOM | 351 | NE | ARG | A | 367 | 36.038 | 48.758 | 68.436 | 1.00 | 36.56 | N |
| ATOM | 352 | CZ | ARG | A | 367 | 36.251 | 49.861 | 67.712 | 1.00 | 37.62 | C |
| ATOM | 353 | NH1 | ARG | A | 367 | 37.465 | 50.156 | 67.255 | 1.00 | 38.38 | N |
| ATOM | 354 | NH2 | ARG | A | 367 | 35.239 | 50.676 | 67.438 | 1.00 | 37.68 | N |
| ATOM | 355 | N | LEU | A | 368 | 38.258 | 43.507 | 66.865 | 1.00 | 34.18 | N |
| ATOM | 356 | CA | LEU | A | 368 | 39.621 | 43.013 | 66.690 | 1.00 | 34.76 | C |
| ATOM | 357 | C | LEU | A | 368 | 39.618 | 41.567 | 67.136 | 1.00 | 34.62 | C |
| ATOM | 358 | O | LEU | A | 368 | 40.381 | 41.211 | 68.055 | 1.00 | 35.34 | O |
| ATOM | 359 | CB | LEU | A | 368 | 40.577 | 43.815 | 67.604 | 1.00 | 35.04 | C |
| ATOM | 360 | CG | LEU | A | 368 | 40.255 | 44.215 | 69.076 | 1.00 | 35.47 | C |
| ATOM | 361 | CD1 | LEU | A | 368 | 39.579 | 43.129 | 69.942 | 1.00 | 35.53 | C |
| ATOM | 362 | CD2 | LEU | A | 368 | 41.515 | 44.703 | 69.783 | 1.00 | 35.36 | C |
| ATOM | 363 | N | HIS | A | 369 | 38.929 | 40.666 | 66.436 | 1.00 | 33.93 | N |
| ATOM | 364 | CA | HIS | A | 369 | 39.323 | 40.134 | 65.150 | 1.00 | 32.84 | C |
| ATOM | 365 | C | HIS | A | 369 | 38.123 | 39.426 | 64.500 | 1.00 | 32.11 | C |
| ATOM | 366 | O | HIS | A | 369 | 36.996 | 39.907 | 64.578 | 1.00 | 32.24 | O |
| ATOM | 367 | CB | HIS | A | 369 | 40.279 | 39.017 | 65.536 | 1.00 | 32.98 | C |
| ATOM | 368 | CG | HIS | A | 369 | 39.838 | 38.310 | 66.786 | 1.00 | 32.77 | C |
| ATOM | 369 | ND1 | HIS | A | 369 | 38.788 | 37.416 | 66.803 | 1.00 | 32.94 | N |
| ATOM | 370 | CD2 | HIS | A | 369 | 40.230 | 38.448 | 68.075 | 1.00 | 32.59 | C |
| ATOM | 371 | CE1 | HIS | A | 369 | 38.588 | 36.995 | 68.039 | 1.00 | 32.76 | C |
| ATOM | 372 | NE2 | HIS | A | 369 | 39.449 | 37.608 | 68.831 | 1.00 | 32.77 | N |
| ATOM | 373 | N | SER | A | 370 | 38.364 | 38.249 | 63.923 | 1.00 | 30.95 | N |
| ATOM | 374 | CA | SER | A | 370 | 37.355 | 37.536 | 63.129 | 1.00 | 29.97 | C |
| ATOM | 375 | C | SER | A | 370 | 36.725 | 36.290 | 63.787 | 1.00 | 29.37 | C |
| ATOM | 376 | O | SER | A | 370 | 35.760 | 35.730 | 63.270 | 1.00 | 29.13 | O |
| ATOM | 377 | CB | SER | A | 370 | 37.955 | 37.165 | 61.770 | 1.00 | 29.69 | C |
| ATOM | 378 | OG | SER | A | 370 | 36.954 | 36.752 | 60.872 | 1.00 | 29.06 | O |
| ATOM | 379 | N | SER | A | 371 | 37.258 | 35.858 | 64.924 | 1.00 | 28.82 | N |
| ATOM | 380 | CA | SER | A | 371 | 36.833 | 34.589 | 65.513 | 1.00 | 28.07 | C |
| ATOM | 381 | C | SER | A | 371 | 35.525 | 34.698 | 66.298 | 1.00 | 27.81 | C |
| ATOM | 382 | O | SER | A | 371 | 34.994 | 35.797 | 66.498 | 1.00 | 27.91 | O |
| ATOM | 383 | CB | SER | A | 371 | 37.951 | 33.989 | 66.366 | 1.00 | 28.06 | C |
| ATOM | 384 | OG | SER | A | 371 | 39.005 | 33.512 | 65.544 | 1.00 | 28.00 | O |
| ATOM | 385 | N | ASP | A | 372 | 35.016 | 33.555 | 66.742 | 1.00 | 26.99 | N |
| ATOM | 386 | CA | ASP | A | 372 | 33.728 | 33.490 | 67.413 | 1.00 | 26.46 | C |
| ATOM | 387 | C | ASP | A | 372 | 33.764 | 33.941 | 68.885 | 1.00 | 26.44 | C |
| ATOM | 388 | O | ASP | A | 372 | 33.114 | 33.342 | 69.742 | 1.00 | 26.31 | O |
| ATOM | 389 | CB | ASP | A | 372 | 33.143 | 32.080 | 67.264 | 1.00 | 26.30 | C |
| ATOM | 390 | CG | ASP | A | 372 | 32.797 | 31.740 | 65.812 | 1.00 | 25.70 | C |
| ATOM | 391 | OD1 | ASP | A | 372 | 32.637 | 32.676 | 65.000 | 1.00 | 23.97 | O |
| ATOM | 392 | OD2 | ASP | A | 372 | 32.680 | 30.538 | 65.485 | 1.00 | 24.88 | O |
| ATOM | 393 | N | TRP | A | 373 | 34.518 | 35.006 | 69.162 | 1.00 | 26.28 | N |
| ATOM | 394 | CA | TRP | A | 373 | 34.634 | 35.579 | 70.514 | 1.00 | 26.40 | C |
| ATOM | 395 | C | TRP | A | 373 | 35.249 | 36.979 | 70.455 | 1.00 | 26.58 | C |
| ATOM | 396 | O | TRP | A | 373 | 35.599 | 37.464 | 69.373 | 1.00 | 26.63 | O |
| ATOM | 397 | CB | TRP | A | 373 | 35.470 | 34.681 | 71.442 | 1.00 | 26.12 | C |
| ATOM | 398 | CG | TRP | A | 373 | 36.812 | 34.285 | 70.878 | 1.00 | 26.06 | C |
| ATOM | 399 | CD1 | TRP | A | 373 | 37.965 | 35.016 | 70.918 | 1.00 | 25.93 | C |
| ATOM | 400 | CD2 | TRP | A | 373 | 37.135 | 33.062 | 70.194 | 1.00 | 26.02 | C |
| ATOM | 401 | NE1 | TRP | A | 373 | 38.988 | 34.330 | 70.299 | 1.00 | 26.45 | N |
| ATOM | 402 | CE2 | TRP | A | 373 | 38.508 | 33.128 | 69.846 | 1.00 | 26.76 | C |
| ATOM | 403 | CE3 | TRP | A | 373 | 36.401 | 31.921 | 69.840 | 1.00 | 25.43 | C |
| ATOM | 404 | CZ2 | TRP | A | 373 | 39.165 | 32.089 | 69.160 | 1.00 | 26.16 | C |
| ATOM | 405 | CZ3 | TRP | A | 373 | 37.052 | 30.887 | 69.156 | 1.00 | 26.22 | C |
| ATOM | 406 | CH2 | TRP | A | 373 | 38.423 | 30.982 | 68.822 | 1.00 | 26.10 | C |
| ATOM | 407 | N | GLY | A | 374 | 35.371 | 37.622 | 71.616 | 1.00 | 26.49 | N |
| ATOM | 408 | CA | GLY | A | 374 | 36.062 | 38.903 | 71.711 | 1.00 | 26.73 | C |
| ATOM | 409 | C | GLY | A | 374 | 35.142 | 40.085 | 71.940 | 1.00 | 26.85 | C |
| ATOM | 410 | O | GLY | A | 374 | 34.050 | 39.943 | 72.489 | 1.00 | 26.64 | O |
| ATOM | 411 | N | ASP | A | 375 | 35.608 | 41.263 | 71.537 | 1.00 | 27.07 | N |
| ATOM | 412 | CA | ASP | A | 375 | 34.787 | 42.460 | 71.569 | 1.00 | 27.15 | C |
| ATOM | 413 | C | ASP | A | 375 | 33.867 | 42.383 | 70.361 | 1.00 | 27.15 | C |
| ATOM | 414 | O | ASP | A | 375 | 34.283 | 42.602 | 69.221 | 1.00 | 27.30 | O |
| ATOM | 415 | CB | ASP | A | 375 | 35.658 | 43.720 | 71.538 | 1.00 | 27.22 | C |
| ATOM | 416 | CG | ASP | A | 375 | 34.857 | 45.000 | 71.746 | 1.00 | 27.62 | C |
| ATOM | 417 | OD1 | ASP | A | 375 | 33.613 | 44.934 | 71.865 | 1.00 | 28.63 | O |
| ATOM | 418 | OD2 | ASP | A | 375 | 35.477 | 46.084 | 71.789 | 1.00 | 27.44 | O |
| ATOM | 419 | N | ILE | A | 376 | 32.622 | 42.006 | 70.624 | 1.00 | 27.16 | N |
| ATOM | 420 | CA | ILE | A | 376 | 31.613 | 41.896 | 69.592 | 1.00 | 26.89 | C |
| ATOM | 421 | C | ILE | A | 376 | 30.377 | 42.642 | 70.057 | 1.00 | 26.85 | C |
| ATOM | 422 | O | ILE | A | 376 | 29.910 | 42.447 | 71.177 | 1.00 | 27.03 | O |
| ATOM | 423 | CB | ILE | A | 376 | 31.254 | 40.427 | 69.271 | 1.00 | 26.88 | C |
| ATOM | 424 | CG1 | ILE | A | 376 | 32.512 | 39.608 | 68.969 | 1.00 | 26.64 | C |
| ATOM | 425 | CG2 | ILE | A | 376 | 30.290 | 40.365 | 68.084 | 1.00 | 26.87 | C |
| ATOM | 426 | CD1 | ILE | A | 376 | 32.265 | 38.120 | 68.769 | 1.00 | 26.87 | C |
| ATOM | 427 | N | GLY | A | 377 | 29.871 | 43.517 | 69.196 | 1.00 | 26.82 | N |
| ATOM | 428 | CA | GLY | A | 377 | 28.616 | 44.218 | 69.455 | 1.00 | 26.46 | C |
| ATOM | 429 | C | GLY | A | 377 | 27.549 | 43.758 | 68.486 | 1.00 | 26.10 | C |
| ATOM | 430 | O | GLY | A | 377 | 27.861 | 43.285 | 67.387 | 1.00 | 25.96 | O |
| ATOM | 431 | N | MET | A | 378 | 26.291 | 43.867 | 68.904 | 1.00 | 26.01 | N |
| ATOM | 432 | CA | MET | A | 378 | 25.154 | 43.602 | 68.018 | 1.00 | 25.87 | C |
| ATOM | 433 | C | MET | A | 378 | 24.736 | 44.936 | 67.420 | 1.00 | 25.89 | C |
| ATOM | 434 | O | MET | A | 378 | 24.327 | 45.850 | 68.148 | 1.00 | 25.93 | O |
| ATOM | 435 | CB | MET | A | 378 | 23.982 | 42.976 | 68.775 | 1.00 | 25.73 | C |
| ATOM | 436 | CG | MET | A | 378 | 24.317 | 41.738 | 69.586 | 1.00 | 26.06 | C |
| ATOM | 437 | SD | MET | A | 378 | 24.606 | 40.270 | 68.578 | 1.00 | 26.92 | S |
| ATOM | 438 | CE | MET | A | 378 | 26.378 | 40.361 | 68.337 | 1.00 | 25.08 | C |
| ATOM | 439 | N | VAL | A | 379 | 24.867 | 45.049 | 66.100 | 1.00 | 25.62 | N |
| ATOM | 440 | CA | VAL | A | 379 | 24.628 | 46.309 | 65.400 | 1.00 | 25.63 | C |
| ATOM | 441 | C | VAL | A | 379 | 23.557 | 46.144 | 64.333 | 1.00 | 25.64 | C |
| ATOM | 442 | O | VAL | A | 379 | 23.428 | 45.077 | 63.735 | 1.00 | 25.94 | O |
| ATOM | 443 | CB | VAL | A | 379 | 25.933 | 46.860 | 64.723 | 1.00 | 25.51 | C |
| ATOM | 444 | CG1 | VAL | A | 379 | 26.968 | 47.233 | 65.755 | 1.00 | 25.29 | C |
| ATOM | 445 | CG2 | VAL | A | 379 | 26.524 | 45.848 | 63.743 | 1.00 | 25.59 | C |
| ATOM | 446 | N | ILE | A | 380 | 22.788 | 47.197 | 64.091 | 1.00 | 25.68 | N |
| ATOM | 447 | CA | ILE | A | 380 | 21.940 | 47.234 | 62.900 | 1.00 | 25.87 | C |
| ATOM | 448 | C | ILE | A | 380 | 22.167 | 48.491 | 62.068 | 1.00 | 26.24 | C |
| ATOM | 449 | O | ILE | A | 380 | 22.762 | 49.465 | 62.535 | 1.00 | 26.21 | O |
| ATOM | 450 | CB | ILE | A | 380 | 20.405 | 47.030 | 63.195 | 1.00 | 25.77 | C |
| ATOM | 451 | CG1 | ILE | A | 380 | 19.844 | 48.126 | 64.101 | 1.00 | 24.95 | C |
| ATOM | 452 | CG2 | ILE | A | 380 | 20.116 | 45.627 | 63.740 | 1.00 | 25.80 | C |
| ATOM | 453 | CD1 | ILE | A | 380 | 19.191 | 49.270 | 63.342 | 1.00 | 25.56 | C |
| ATOM | 454 | N | ARG | A | 381 | 21.711 | 48.430 | 60.822 | 1.00 | 26.76 | N |
| ATOM | 455 | CA | ARG | A | 381 | 21.576 | 49.593 | 59.957 | 1.00 | 27.12 | C |
| ATOM | 456 | C | ARG | A | 381 | 20.183 | 49.553 | 59.337 | 1.00 | 27.37 | C |
| ATOM | 457 | O | ARG | A | 381 | 19.595 | 48.479 | 59.189 | 1.00 | 27.09 | O |
| ATOM | 458 | CB | ARG | A | 381 | 22.640 | 49.587 | 58.866 | 1.00 | 27.08 | C |
| ATOM | 459 | CG | ARG | A | 381 | 24.045 | 49.867 | 59.369 | 1.00 | 27.23 | C |
| ATOM | 460 | CD | ARG | A | 381 | 25.014 | 50.059 | 58.210 | 1.00 | 27.34 | C |
| ATOM | 461 | NE | ARG | A | 381 | 24.865 | 51.367 | 57.578 | 1.00 | 27.64 | N |
| ATOM | 462 | CZ | ARG | A | 381 | 25.474 | 51.743 | 56.454 | 1.00 | 28.85 | C |
| ATOM | 463 | NH1 | ARG | A | 381 | 26.281 | 50.905 | 55.806 | 1.00 | 28.43 | N |
| ATOM | 464 | NH2 | ARG | A | 381 | 25.264 | 52.964 | 55.967 | 1.00 | 28.40 | N |
| ATOM | 465 | N | ARG | A | 382 | 19.665 | 50.729 | 58.988 | 1.00 | 27.77 | N |
| ATOM | 466 | CA | ARG | A | 382 | 18.313 | 50.866 | 58.451 | 1.00 | 28.21 | C |
| ATOM | 467 | C | ARG | A | 382 | 18.303 | 51.440 | 57.039 | 1.00 | 28.60 | C |
| ATOM | 468 | O | ARG | A | 382 | 19.079 | 52.343 | 56.722 | 1.00 | 28.73 | O |
| ATOM | 469 | CB | ARG | A | 382 | 17.479 | 51.774 | 59.353 | 1.00 | 28.02 | C |
| ATOM | 470 | CG | ARG | A | 382 | 17.141 | 51.194 | 60.697 | 1.00 | 27.54 | C |
| ATOM | 471 | CD | ARG | A | 382 | 16.434 | 52.213 | 61.580 | 1.00 | 27.37 | C |
| ATOM | 472 | NE | ARG | A | 382 | 15.061 | 52.486 | 61.147 | 1.00 | 27.63 | N |
| ATOM | 473 | CZ | ARG | A | 382 | 14.679 | 53.564 | 60.462 | 1.00 | 27.60 | C |
| ATOM | 474 | NH1 | ARG | A | 382 | 15.562 | 54.494 | 60.112 | 1.00 | 26.94 | N |
| ATOM | 475 | NH2 | ARG | A | 382 | 13.407 | 53.712 | 60.124 | 1.00 | 27.55 | N |
| ATOM | 476 | N | SER | A | 383 | 17.413 | 50.924 | 56.198 | 1.00 | 29.07 | N |
| ATOM | 477 | CA | SER | A | 383 | 17.168 | 51.525 | 54.895 | 1.00 | 29.40 | C |
| ATOM | 478 | C | SER | A | 383 | 15.696 | 51.844 | 54.722 | 1.00 | 29.69 | C |
| ATOM | 479 | O | SER | A | 383 | 14.834 | 51.027 | 55.037 | 1.00 | 29.57 | O |
| ATOM | 480 | CB | SER | A | 383 | 17.627 | 50.617 | 53.763 | 1.00 | 29.44 | C |
| ATOM | 481 | OG | SER | A | 383 | 17.447 | 51.262 | 52.510 | 1.00 | 29.80 | O |
| ATOM | 482 | N | GLU | A | 384 | 15.423 | 53.036 | 54.204 | 1.00 | 30.22 | N |
| ATOM | 483 | CA | GLU | A | 384 | 14.058 | 53.467 | 53.937 | 1.00 | 30.89 | C |
| ATOM | 484 | C | GLU | A | 384 | 13.771 | 53.506 | 52.439 | 1.00 | 30.86 | C |
| ATOM | 485 | O | GLU | A | 384 | 12.621 | 53.431 | 52.023 | 1.00 | 31.24 | O |
| ATOM | 486 | CB | GLU | A | 384 | 13.781 | 54.821 | 54.597 | 1.00 | 30.80 | C |
| ATOM | 487 | CG | GLU | A | 384 | 14.126 | 54.850 | 56.088 | 1.00 | 31.30 | C |
| ATOM | 488 | CD | GLU | A | 384 | 13.552 | 56.051 | 56.820 | 1.00 | 31.86 | C |
| ATOM | 489 | OE1 | GLU | A | 384 | 13.309 | 57.095 | 56.174 | 1.00 | 33.40 | O |
| ATOM | 490 | OE2 | GLU | A | 384 | 13.346 | 55.950 | 58.052 | 1.00 | 33.27 | O |
| ATOM | 491 | N | ASP | A | 385 | 14.818 | 53.606 | 51.628 | 1.00 | 31.13 | N |
| ATOM | 492 | CA | ASP | A | 385 | 14.658 | 53.556 | 50.172 | 1.00 | 31.34 | C |
| ATOM | 493 | C | ASP | A | 385 | 14.805 | 52.123 | 49.664 | 1.00 | 31.26 | C |
| ATOM | 494 | O | ASP | A | 385 | 15.277 | 51.879 | 48.550 | 1.00 | 31.16 | O |
| ATOM | 495 | CB | ASP | A | 385 | 15.618 | 54.522 | 49.460 | 1.00 | 31.18 | C |
| ATOM | 496 | CG | ASP | A | 385 | 17.071 | 54.332 | 49.874 | 1.00 | 32.28 | C |
| ATOM | 497 | OD1 | ASP | A | 385 | 17.382 | 53.392 | 50.644 | 1.00 | 33.22 | O |
| ATOM | 498 | OD2 | ASP | A | 385 | 17.912 | 55.135 | 49.414 | 1.00 | 32.58 | O |
| ATOM | 499 | N | ASN | A | 386 | 14.383 | 51.185 | 50.507 | 1.00 | 31.44 | N |
| ATOM | 500 | CA | ASN | A | 386 | 14.347 | 49.765 | 50.177 | 1.00 | 31.44 | C |
| ATOM | 501 | C | ASN | A | 386 | 15.694 | 49.228 | 49.673 | 1.00 | 31.55 | C |
| ATOM | 502 | O | ASN | A | 386 | 15.761 | 48.587 | 48.631 | 1.00 | 31.54 | O |
| ATOM | 503 | CB | ASN | A | 386 | 13.220 | 49.488 | 49.172 | 1.00 | 31.30 | C |
| ATOM | 504 | CG | ASN | A | 386 | 12.712 | 48.063 | 49.242 | 1.00 | 31.04 | C |
| ATOM | 505 | OD1 | ASN | A | 386 | 12.336 | 47.576 | 50.309 | 1.00 | 31.42 | O |
| ATOM | 506 | ND2 | ASN | A | 386 | 12.687 | 47.390 | 48.102 | 1.00 | 29.95 | N |
| ATOM | 507 | N | GLY | A | 387 | 16.765 | 49.519 | 50.408 | 1.00 | 31.77 | N |
| ATOM | 508 | CA | GLY | A | 387 | 18.075 | 48.931 | 50.132 | 1.00 | 32.05 | C |
| ATOM | 509 | C | GLY | A | 387 | 19.057 | 49.724 | 49.286 | 1.00 | 32.33 | C |
| ATOM | 510 | O | GLY | A | 387 | 20.197 | 49.292 | 49.098 | 1.00 | 32.56 | O |
| ATOM | 511 | N | LYS | A | 388 | 18.636 | 50.879 | 48.776 | 1.00 | 32.47 | N |
| ATOM | 512 | CA | LYS | A | 388 | 19.522 | 51.715 | 47.961 | 1.00 | 32.65 | C |
| ATOM | 513 | C | LYS | A | 388 | 20.579 | 52.404 | 48.822 | 1.00 | 32.41 | C |
| ATOM | 514 | O | LYS | A | 388 | 21.776 | 52.251 | 48.577 | 1.00 | 32.31 | O |
| ATOM | 515 | CB | LYS | A | 388 | 18.729 | 52.744 | 47.148 | 1.00 | 32.87 | C |
| ATOM | 516 | CG | LYS | A | 388 | 17.852 | 52.130 | 46.071 | 1.00 | 34.42 | C |
| ATOM | 517 | CD | LYS | A | 388 | 16.941 | 53.165 | 45.423 | 1.00 | 36.60 | C |
| ATOM | 518 | CE | LYS | A | 388 | 16.009 | 52.500 | 44.416 | 1.00 | 37.43 | C |
| ATOM | 519 | NZ | LYS | A | 388 | 15.032 | 53.461 | 43.836 | 1.00 | 38.43 | N |
| ATOM | 520 | N | THR | A | 389 | 20.122 | 53.164 | 49.818 | 1.00 | 32.11 | N |
| ATOM | 521 | CA | THR | A | 389 | 21.004 | 53.819 | 50.785 | 1.00 | 31.69 | C |
| ATOM | 522 | C | THR | A | 389 | 20.696 | 53.306 | 52.186 | 1.00 | 31.24 | C |
| ATOM | 523 | O | THR | A | 389 | 19.598 | 52.811 | 52.448 | 1.00 | 31.38 | O |
| ATOM | 524 | CB | THR | A | 389 | 20.877 | 55.366 | 50.759 | 1.00 | 31.91 | C |
| ATOM | 525 | OG1 | THR | A | 389 | 19.557 | 55.757 | 51.162 | 1.00 | 31.95 | O |
| ATOM | 526 | CG2 | THR | A | 389 | 21.183 | 55.926 | 49.368 | 1.00 | 31.62 | C |
| ATOM | 527 | N | TRP | A | 390 | 21.670 | 53.418 | 53.082 | 1.00 | 30.59 | N |
| ATOM | 528 | CA | TRP | A | 390 | 21.539 | 52.867 | 54.419 | 1.00 | 30.15 | C |
| ATOM | 529 | C | TRP | A | 390 | 22.022 | 53.888 | 55.431 | 1.00 | 30.11 | C |
| ATOM | 530 | O | TRP | A | 390 | 23.006 | 54.587 | 55.182 | 1.00 | 29.99 | O |
| ATOM | 531 | CB | TRP | A | 390 | 22.319 | 51.550 | 54.542 | 1.00 | 29.91 | C |
| ATOM | 532 | CG | TRP | A | 390 | 21.814 | 50.459 | 53.621 | 1.00 | 29.72 | C |
| ATOM | 533 | CD1 | TRP | A | 390 | 22.061 | 50.337 | 52.281 | 1.00 | 29.74 | C |
| ATOM | 534 | CD2 | TRP | A | 390 | 20.975 | 49.346 | 53.974 | 1.00 | 29.99 | C |
| ATOM | 535 | NE1 | TRP | A | 390 | 21.425 | 49.224 | 51.776 | 1.00 | 30.06 | N |
| ATOM | 536 | CE2 | TRP | A | 390 | 20.753 | 48.597 | 52.793 | 1.00 | 29.99 | C |
| ATOM | 537 | CE3 | TRP | A | 390 | 20.393 | 48.905 | 55.175 | 1.00 | 29.73 | C |
| ATOM | 538 | CZ2 | TRP | A | 390 | 19.976 | 47.430 | 52.777 | 1.00 | 29.68 | C |
| ATOM | 539 | CZ3 | TRP | A | 390 | 19.616 | 47.749 | 55.156 | 1.00 | 29.62 | C |
| ATOM | 540 | CH2 | TRP | A | 390 | 19.419 | 47.024 | 53.964 | 1.00 | 29.94 | C |
| ATOM | 541 | N | GLY | A | 391 | 21.322 | 53.969 | 56.563 | 1.00 | 29.91 | N |
| ATOM | 542 | CA | GLY | A | 391 | 21.615 | 54.951 | 57.608 | 1.00 | 29.89 | C |
| ATOM | 543 | C | GLY | A | 391 | 22.840 | 54.619 | 58.442 | 1.00 | 29.93 | C |
| ATOM | 544 | O | GLY | A | 391 | 23.709 | 53.862 | 58.009 | 1.00 | 29.81 | O |
| ATOM | 545 | N | ASP | A | 392 | 22.908 | 55.195 | 59.641 | 1.00 | 29.99 | N |
| ATOM | 546 | CA | ASP | A | 392 | 24.019 | 54.959 | 60.563 | 1.00 | 30.27 | C |
| ATOM | 547 | C | ASP | A | 392 | 23.923 | 53.598 | 61.241 | 1.00 | 30.61 | C |
| ATOM | 548 | O | ASP | A | 392 | 22.838 | 53.034 | 61.376 | 1.00 | 30.75 | O |
| ATOM | 549 | CB | ASP | A | 392 | 24.068 | 56.051 | 61.640 | 1.00 | 30.30 | C |
| ATOM | 550 | CG | ASP | A | 392 | 24.459 | 57.414 | 61.085 | 1.00 | 30.07 | C |
| ATOM | 551 | OD1 | ASP | A | 392 | 25.231 | 57.472 | 60.108 | 1.00 | 29.41 | O |
| ATOM | 552 | OD2 | ASP | A | 392 | 23.994 | 58.435 | 61.631 | 1.00 | 30.16 | O |
| ATOM | 553 | N | ARG | A | 393 | 25.067 | 53.078 | 61.672 | 1.00 | 30.83 | N |
| ATOM | 554 | CA | ARG | A | 393 | 25.100 | 51.872 | 62.485 | 1.00 | 31.07 | C |
| ATOM | 555 | C | ARG | A | 393 | 24.545 | 52.195 | 63.869 | 1.00 | 30.97 | C |
| ATOM | 556 | O | ARG | A | 393 | 25.058 | 53.078 | 64.564 | 1.00 | 31.12 | O |
| ATOM | 557 | CB | ARG | A | 393 | 26.529 | 51.322 | 62.592 | 1.00 | 30.93 | C |
| ATOM | 558 | CG | ARG | A | 393 | 27.152 | 50.940 | 61.259 | 1.00 | 31.32 | C |
| ATOM | 559 | CD | ARG | A | 393 | 28.628 | 50.574 | 61.387 | 1.00 | 31.99 | C |
| ATOM | 560 | NE | ARG | A | 393 | 29.465 | 51.745 | 61.645 | 1.00 | 34.32 | N |
| ATOM | 561 | CZ | ARG | A | 393 | 30.217 | 51.911 | 62.728 | 1.00 | 35.30 | C |
| ATOM | 562 | NH1 | ARG | A | 393 | 30.268 | 50.974 | 63.663 | 1.00 | 36.31 | N |
| ATOM | 563 | NH2 | ARG | A | 393 | 30.933 | 53.016 | 62.874 | 1.00 | 36.01 | N |
| ATOM | 564 | N | VAL | A | 394 | 23.478 | 51.493 | 64.248 | 1.00 | 30.78 | N |
| ATOM | 565 | CA | VAL | A | 394 | 22.910 | 51.594 | 65.589 | 1.00 | 30.37 | C |
| ATOM | 566 | C | VAL | A | 394 | 23.353 | 50.365 | 66.379 | 1.00 | 30.14 | C |
| ATOM | 567 | O | VAL | A | 394 | 23.014 | 49.242 | 66.030 | 1.00 | 30.25 | O |
| ATOM | 568 | CB | VAL | A | 394 | 21.350 | 51.697 | 65.559 | 1.00 | 30.44 | C |
| ATOM | 569 | CG1 | VAL | A | 394 | 20.762 | 51.531 | 66.950 | 1.00 | 30.63 | C |
| ATOM | 570 | CG2 | VAL | A | 394 | 20.901 | 53.026 | 64.964 | 1.00 | 29.99 | C |
| ATOM | 571 | N | THR | A | 395 | 24.129 | 50.589 | 67.432 | 1.00 | 30.03 | N |
| ATOM | 572 | CA | THR | A | 395 | 24.567 | 49.511 | 68.308 | 1.00 | 29.72 | C |
| ATOM | 573 | C | THR | A | 395 | 23.466 | 49.164 | 69.305 | 1.00 | 29.69 | C |
| ATOM | 574 | O | THR | A | 395 | 23.027 | 50.012 | 70.089 | 1.00 | 29.73 | O |
| ATOM | 575 | CB | THR | A | 395 | 25.858 | 49.885 | 69.065 | 1.00 | 29.76 | C |
| ATOM | 576 | OG1 | THR | A | 395 | 26.867 | 50.265 | 68.122 | 1.00 | 29.58 | O |
| ATOM | 577 | CG2 | THR | A | 395 | 26.365 | 48.696 | 69.894 | 1.00 | 29.36 | C |
| ATOM | 578 | N | ILE | A | 396 | 23.015 | 47.915 | 69.260 | 1.00 | 29.40 | N |
| ATOM | 579 | CA | ILE | A | 396 | 21.982 | 47.445 | 70.166 | 1.00 | 29.08 | C |
| ATOM | 580 | C | ILE | A | 396 | 22.631 | 47.128 | 71.512 | 1.00 | 28.98 | C |
| ATOM | 581 | O | ILE | A | 396 | 22.198 | 47.618 | 72.555 | 1.00 | 28.73 | O |
| ATOM | 582 | CB | ILE | A | 396 | 21.245 | 46.199 | 69.604 | 1.00 | 29.20 | C |
| ATOM | 583 | CG1 | ILE | A | 396 | 20.733 | 46.460 | 68.177 | 1.00 | 29.44 | C |
| ATOM | 584 | CG2 | ILE | A | 396 | 20.109 | 45.782 | 70.537 | 1.00 | 28.85 | C |
| ATOM | 585 | CD1 | ILE | A | 396 | 20.025 | 45.270 | 67.519 | 1.00 | 28.92 | C |
| ATOM | 586 | N | THR | A | 397 | 23.676 | 46.307 | 71.474 | 1.00 | 29.01 | N |
| ATOM | 587 | CA | THR | A | 397 | 24.390 | 45.905 | 72.678 | 1.00 | 29.06 | C |
| ATOM | 588 | C | THR | A | 397 | 25.857 | 45.710 | 72.365 | 1.00 | 29.29 | C |
| ATOM | 589 | O | THR | A | 397 | 26.204 | 45.037 | 71.398 | 1.00 | 29.42 | O |
| ATOM | 590 | CB | THR | A | 397 | 23.844 | 44.573 | 73.277 | 1.00 | 29.04 | C |
| ATOM | 591 | OG1 | THR | A | 397 | 22.411 | 44.585 | 73.292 | 1.00 | 28.28 | O |
| ATOM | 592 | CG2 | THR | A | 397 | 24.366 | 44.367 | 74.694 | 1.00 | 28.30 | C |
| ATOM | 593 | N | ASN | A | 398 | 26.706 | 46.332 | 73.174 | 1.00 | 29.71 | N |
| ATOM | 594 | CA | ASN | A | 398 | 28.119 | 45.987 | 73.234 | 1.00 | 30.16 | C |
| ATOM | 595 | C | ASN | A | 398 | 28.574 | 46.089 | 74.676 | 1.00 | 30.41 | C |
| ATOM | 596 | O | ASN | A | 398 | 28.649 | 47.182 | 75.229 | 1.00 | 30.44 | O |
| ATOM | 597 | CB | ASN | A | 398 | 28.968 | 46.892 | 72.336 | 1.00 | 29.97 | C |
| ATOM | 598 | CG | ASN | A | 398 | 30.362 | 46.316 | 72.061 | 1.00 | 30.47 | C |
| ATOM | 599 | OD1 | ASN | A | 398 | 30.812 | 45.359 | 72.706 | 1.00 | 29.87 | O |
| ATOM | 600 | ND2 | ASN | A | 398 | 31.052 | 46.908 | 71.094 | 1.00 | 30.90 | N |
| ATOM | 601 | N | LEU | A | 399 | 28.846 | 44.941 | 75.285 | 1.00 | 31.04 | N |
| ATOM | 602 | CA | LEU | A | 399 | 29.321 | 44.891 | 76.664 | 1.00 | 31.74 | C |
| ATOM | 603 | C | LEU | A | 399 | 30.771 | 45.348 | 76.715 | 1.00 | 32.40 | C |
| ATOM | 604 | O | LEU | A | 399 | 31.552 | 45.038 | 75.815 | 1.00 | 32.19 | O |
| ATOM | 605 | CB | LEU | A | 399 | 29.171 | 43.476 | 77.233 | 1.00 | 31.69 | C |
| ATOM | 606 | CG | LEU | A | 399 | 27.756 | 42.873 | 77.184 | 1.00 | 31.25 | C |
| ATOM | 607 | CD1 | LEU | A | 399 | 27.756 | 41.393 | 77.562 | 1.00 | 29.67 | C |
| ATOM | 608 | CD2 | LEU | A | 399 | 26.785 | 43.668 | 78.056 | 1.00 | 29.66 | C |
| ATOM | 609 | N | ARG | A | 400 | 31.123 | 46.101 | 77.754 | 1.00 | 33.41 | N |
| ATOM | 610 | CA | ARG | A | 400 | 32.475 | 46.651 | 77.858 | 1.00 | 34.49 | C |
| ATOM | 611 | C | ARG | A | 400 | 33.505 | 45.566 | 78.147 | 1.00 | 35.22 | C |
| ATOM | 612 | O | ARG | A | 400 | 33.221 | 44.611 | 78.874 | 1.00 | 35.31 | O |
| ATOM | 613 | CB | ARG | A | 400 | 32.545 | 47.772 | 78.895 | 1.00 | 34.35 | C |
| ATOM | 614 | CG | ARG | A | 400 | 32.412 | 47.330 | 80.338 | 1.00 | 34.53 | C |
| ATOM | 615 | CD | ARG | A | 400 | 32.211 | 48.542 | 81.231 | 1.00 | 34.00 | C |
| ATOM | 616 | NE | ARG | A | 400 | 32.393 | 48.217 | 82.639 | 1.00 | 33.98 | N |
| ATOM | 617 | CZ | ARG | A | 400 | 33.568 | 48.196 | 83.260 | 1.00 | 33.20 | C |
| ATOM | 618 | NH1 | ARG | A | 400 | 34.683 | 48.475 | 82.598 | 1.00 | 32.29 | N |
| ATOM | 619 | NH2 | ARG | A | 400 | 33.624 | 47.888 | 84.547 | 1.00 | 32.93 | N |
| ATOM | 620 | N | ASP | A | 401 | 34.690 | 45.715 | 77.560 | 1.00 | 36.17 | N |
| ATOM | 621 | CA | ASP | A | 401 | 35.758 | 44.734 | 77.734 | 1.00 | 37.21 | C |
| ATOM | 622 | C | ASP | A | 401 | 36.334 | 44.773 | 79.146 | 1.00 | 37.58 | C |
| ATOM | 623 | O | ASP | A | 401 | 36.162 | 45.756 | 79.868 | 1.00 | 37.55 | O |
| ATOM | 624 | CB | ASP | A | 401 | 36.867 | 44.931 | 76.688 | 1.00 | 37.32 | C |
| ATOM | 625 | CG | ASP | A | 401 | 37.592 | 46.265 | 76.826 | 1.00 | 38.42 | C |
| ATOM | 626 | OD1 | ASP | A | 401 | 37.111 | 47.164 | 77.553 | 1.00 | 39.60 | O |
| ATOM | 627 | OD2 | ASP | A | 401 | 38.654 | 46.421 | 76.188 | 1.00 | 40.20 | O |
| ATOM | 628 | N | ASN | A | 402 | 36.990 | 43.683 | 79.534 | 1.00 | 38.33 | N |
| ATOM | 629 | CA | ASN | A | 402 | 37.743 | 43.634 | 80.773 | 1.00 | 39.07 | C |
| ATOM | 630 | C | ASN | A | 402 | 39.088 | 44.332 | 80.572 | 1.00 | 39.53 | C |
| ATOM | 631 | O | ASN | A | 402 | 39.987 | 43.765 | 79.940 | 1.00 | 39.49 | O |
| ATOM | 632 | CB | ASN | A | 402 | 37.938 | 42.185 | 81.222 | 1.00 | 39.26 | C |
| ATOM | 633 | CG | ASN | A | 402 | 38.651 | 42.071 | 82.560 | 1.00 | 39.57 | C |
| ATOM | 634 | OD1 | ASN | A | 402 | 39.394 | 42.967 | 82.968 | 1.00 | 40.75 | O |
| ATOM | 635 | ND2 | ASN | A | 402 | 38.433 | 40.960 | 83.246 | 1.00 | 39.63 | N |
| ATOM | 636 | N | PRO | A | 403 | 39.238 | 45.552 | 81.127 | 1.00 | 39.98 | N |
| ATOM | 637 | CA | PRO | A | 403 | 40.410 | 46.394 | 80.866 | 1.00 | 40.36 | C |
| ATOM | 638 | C | PRO | A | 403 | 41.695 | 45.803 | 81.439 | 1.00 | 40.65 | C |
| ATOM | 639 | O | PRO | A | 403 | 42.784 | 46.239 | 81.071 | 1.00 | 40.74 | O |
| ATOM | 640 | CB | PRO | A | 403 | 40.076 | 47.707 | 81.591 | 1.00 | 40.33 | C |
| ATOM | 641 | CG | PRO | A | 403 | 38.597 | 47.649 | 81.865 | 1.00 | 40.40 | C |
| ATOM | 642 | CD | PRO | A | 403 | 38.314 | 46.200 | 82.075 | 1.00 | 40.12 | C |
| ATOM | 643 | N | LYS | A | 404 | 41.557 | 44.816 | 82.323 | 1.00 | 40.93 | N |
| ATOM | 644 | CA | LYS | A | 404 | 42.700 | 44.216 | 83.008 | 1.00 | 41.05 | C |
| ATOM | 645 | C | LYS | A | 404 | 42.965 | 42.786 | 82.547 | 1.00 | 40.93 | C |
| ATOM | 646 | O | LYS | A | 404 | 43.735 | 42.054 | 83.177 | 1.00 | 40.95 | O |
| ATOM | 647 | CB | LYS | A | 404 | 42.515 | 44.285 | 84.532 | 1.00 | 41.18 | C |
| ATOM | 648 | CG | LYS | A | 404 | 42.646 | 45.697 | 85.113 | 1.00 | 41.88 | C |
| ATOM | 649 | CD | LYS | A | 404 | 44.114 | 46.074 | 85.350 | 1.00 | 43.64 | C |
| ATOM | 650 | CE | LYS | A | 404 | 44.364 | 47.574 | 85.165 | 1.00 | 44.41 | C |
| ATOM | 651 | NZ | LYS | A | 404 | 43.522 | 48.428 | 86.052 | 1.00 | 45.12 | N |
| ATOM | 652 | N | ALA | A | 405 | 42.335 | 42.398 | 81.440 | 1.00 | 40.71 | N |
| ATOM | 653 | CA | ALA | A | 405 | 42.599 | 41.100 | 80.828 | 1.00 | 40.53 | C |
| ATOM | 654 | C | ALA | A | 405 | 43.935 | 41.142 | 80.093 | 1.00 | 40.40 | C |
| ATOM | 655 | O | ALA | A | 405 | 44.244 | 42.124 | 79.405 | 1.00 | 40.42 | O |
| ATOM | 656 | CB | ALA | A | 405 | 41.478 | 40.719 | 79.877 | 1.00 | 40.55 | C |
| ATOM | 657 | N | SER | A | 406 | 44.722 | 40.081 | 80.246 | 1.00 | 40.10 | N |
| ATOM | 658 | CA | SER | A | 406 | 46.044 | 39.993 | 79.619 | 1.00 | 39.82 | C |
| ATOM | 659 | C | SER | A | 406 | 45.986 | 39.793 | 78.096 | 1.00 | 39.72 | C |
| ATOM | 660 | O | SER | A | 406 | 46.813 | 40.347 | 77.366 | 1.00 | 39.83 | O |
| ATOM | 661 | CB | SER | A | 406 | 46.879 | 38.893 | 80.276 | 1.00 | 39.85 | C |
| ATOM | 662 | OG | SER | A | 406 | 46.173 | 37.668 | 80.309 | 1.00 | 39.28 | O |
| ATOM | 663 | N | ASP | A | 407 | 45.017 | 39.002 | 77.630 | 1.00 | 39.22 | N |
| ATOM | 664 | CA | ASP | A | 407 | 44.792 | 38.790 | 76.196 | 1.00 | 38.46 | C |
| ATOM | 665 | C | ASP | A | 407 | 43.480 | 39.458 | 75.784 | 1.00 | 38.10 | C |
| ATOM | 666 | O | ASP | A | 407 | 42.400 | 38.931 | 76.063 | 1.00 | 38.19 | O |
| ATOM | 667 | CB | ASP | A | 407 | 44.779 | 37.287 | 75.867 | 1.00 | 38.37 | C |
| ATOM | 668 | CG | ASP | A | 407 | 44.613 | 36.993 | 74.368 | 1.00 | 37.98 | C |
| ATOM | 669 | OD1 | ASP | A | 407 | 44.326 | 37.920 | 73.571 | 1.00 | 37.46 | O |
| ATOM | 670 | OD2 | ASP | A | 407 | 44.762 | 35.809 | 73.991 | 1.00 | 37.17 | O |
| ATOM | 671 | N | PRO | A | 408 | 43.569 | 40.614 | 75.096 | 1.00 | 37.68 | N |
| ATOM | 672 | CA | PRO | A | 408 | 42.379 | 41.430 | 74.851 | 1.00 | 37.23 | C |
| ATOM | 673 | C | PRO | A | 408 | 41.490 | 40.870 | 73.744 | 1.00 | 36.84 | C |
| ATOM | 674 | O | PRO | A | 408 | 40.331 | 41.266 | 73.633 | 1.00 | 37.06 | O |
| ATOM | 675 | CB | PRO | A | 408 | 42.954 | 42.796 | 74.435 | 1.00 | 37.30 | C |
| ATOM | 676 | CG | PRO | A | 408 | 44.462 | 42.655 | 74.469 | 1.00 | 37.42 | C |
| ATOM | 677 | CD | PRO | A | 408 | 44.770 | 41.199 | 74.473 | 1.00 | 37.68 | C |
| ATOM | 678 | N | SER | A | 409 | 42.030 | 39.960 | 72.935 | 1.00 | 36.22 | N |
| ATOM | 679 | CA | SER | A | 409 | 41.273 | 39.328 | 71.862 | 1.00 | 35.40 | C |
| ATOM | 680 | C | SER | A | 409 | 40.288 | 38.308 | 72.427 | 1.00 | 34.95 | C |
| ATOM | 681 | O | SER | A | 409 | 39.351 | 37.893 | 71.741 | 1.00 | 34.89 | O |
| ATOM | 682 | CB | SER | A | 409 | 42.215 | 38.668 | 70.846 | 1.00 | 35.37 | C |
| ATOM | 683 | OG | SER | A | 409 | 42.730 | 37.442 | 71.329 | 1.00 | 35.18 | O |
| ATOM | 684 | N | ILE | A | 410 | 40.517 | 37.905 | 73.676 | 1.00 | 34.22 | N |
| ATOM | 685 | CA | ILE | A | 410 | 39.608 | 37.017 | 74.403 | 1.00 | 33.59 | C |
| ATOM | 686 | C | ILE | A | 410 | 39.131 | 37.661 | 75.709 | 1.00 | 33.24 | C |
| ATOM | 687 | O | ILE | A | 410 | 38.567 | 36.985 | 76.575 | 1.00 | 33.12 | O |
| ATOM | 688 | CB | ILE | A | 410 | 40.252 | 35.620 | 74.694 | 1.00 | 33.66 | C |
| ATOM | 689 | CG1 | ILE | A | 410 | 41.502 | 35.753 | 75.576 | 1.00 | 33.32 | C |
| ATOM | 690 | CG2 | ILE | A | 410 | 40.565 | 34.878 | 73.385 | 1.00 | 33.85 | C |
| ATOM | 691 | CD1 | ILE | A | 410 | 41.953 | 34.459 | 76.234 | 1.00 | 33.17 | C |
| ATOM | 692 | N | GLY | A | 411 | 39.345 | 38.973 | 75.829 | 1.00 | 32.68 | N |
| ATOM | 693 | CA | GLY | A | 411 | 39.169 | 39.684 | 77.096 | 1.00 | 31.89 | C |
| ATOM | 694 | C | GLY | A | 411 | 37.936 | 40.555 | 77.206 | 1.00 | 31.32 | C |
| ATOM | 695 | O | GLY | A | 411 | 37.949 | 41.594 | 77.872 | 1.00 | 31.07 | O |
| ATOM | 696 | N | SER | A | 412 | 36.861 | 40.122 | 76.558 | 1.00 | 30.95 | N |
| ATOM | 697 | CA | SER | A | 412 | 35.594 | 40.840 | 76.604 | 1.00 | 30.25 | C |
| ATOM | 698 | C | SER | A | 412 | 34.445 | 39.849 | 76.779 | 1.00 | 29.72 | C |
| ATOM | 699 | O | SER | A | 412 | 34.501 | 38.740 | 76.241 | 1.00 | 29.82 | O |
| ATOM | 700 | CB | SER | A | 412 | 35.401 | 41.674 | 75.332 | 1.00 | 30.16 | C |
| ATOM | 701 | OG | SER | A | 412 | 34.274 | 42.530 | 75.440 | 1.00 | 30.49 | O |
| ATOM | 702 | N | PRO | A | 413 | 33.421 | 40.226 | 77.572 | 1.00 | 29.10 | N |
| ATOM | 703 | CA | PRO | A | 413 | 32.151 | 39.500 | 77.543 | 1.00 | 28.57 | C |
| ATOM | 704 | C | PRO | A | 413 | 31.594 | 39.541 | 76.126 | 1.00 | 27.90 | C |
| ATOM | 705 | O | PRO | A | 413 | 31.804 | 40.520 | 75.414 | 1.00 | 27.64 | O |
| ATOM | 706 | CB | PRO | A | 413 | 31.262 | 40.304 | 78.496 | 1.00 | 28.54 | C |
| ATOM | 707 | CG | PRO | A | 413 | 32.214 | 41.008 | 79.400 | 1.00 | 28.65 | C |
| ATOM | 708 | CD | PRO | A | 413 | 33.408 | 41.322 | 78.559 | 1.00 | 28.96 | C |
| ATOM | 709 | N | VAL | A | 414 | 30.899 | 38.484 | 75.720 | 1.00 | 27.49 | N |
| ATOM | 710 | CA | VAL | A | 414 | 30.558 | 38.296 | 74.311 | 1.00 | 26.85 | C |
| ATOM | 711 | C | VAL | A | 414 | 29.055 | 38.322 | 74.042 | 1.00 | 26.29 | C |
| ATOM | 712 | O | VAL | A | 414 | 28.283 | 37.676 | 74.743 | 1.00 | 26.49 | O |
| ATOM | 713 | CB | VAL | A | 414 | 31.149 | 36.963 | 73.757 | 1.00 | 26.98 | C |
| ATOM | 714 | CG1 | VAL | A | 414 | 30.959 | 36.872 | 72.246 | 1.00 | 26.71 | C |
| ATOM | 715 | CG2 | VAL | A | 414 | 32.629 | 36.822 | 74.118 | 1.00 | 26.56 | C |
| ATOM | 716 | N | ASN | A | 415 | 28.660 | 39.098 | 73.036 | 1.00 | 25.59 | N |
| ATOM | 717 | CA | ASN | A | 415 | 27.382 | 38.916 | 72.351 | 1.00 | 24.74 | C |
| ATOM | 718 | C | ASN | A | 415 | 27.679 | 38.409 | 70.941 | 1.00 | 24.22 | C |
| ATOM | 719 | O | ASN | A | 415 | 28.611 | 38.886 | 70.301 | 1.00 | 23.92 | O |
| ATOM | 720 | CB | ASN | A | 415 | 26.597 | 40.228 | 72.299 | 1.00 | 24.63 | C |
| ATOM | 721 | CG | ASN | A | 415 | 26.042 | 40.639 | 73.658 | 1.00 | 24.21 | C |
| ATOM | 722 | OD1 | ASN | A | 415 | 26.264 | 41.762 | 74.112 | 1.00 | 23.56 | O |
| ATOM | 723 | ND2 | ASN | A | 415 | 25.308 | 39.734 | 74.308 | 1.00 | 22.62 | N |
| ATOM | 724 | N | ILE | A | 416 | 26.903 | 37.438 | 70.462 | 1.00 | 23.84 | N |
| ATOM | 725 | CA | ILE | A | 416 | 27.221 | 36.742 | 69.207 | 1.00 | 22.95 | C |
| ATOM | 726 | C | ILE | A | 416 | 25.994 | 36.065 | 68.589 | 1.00 | 23.06 | C |
| ATOM | 727 | O | ILE | A | 416 | 25.214 | 35.426 | 69.303 | 1.00 | 23.14 | O |
| ATOM | 728 | CB | ILE | A | 416 | 28.384 | 35.719 | 69.421 | 1.00 | 22.83 | C |
| ATOM | 729 | CG1 | ILE | A | 416 | 28.725 | 34.966 | 68.128 | 1.00 | 22.48 | C |
| ATOM | 730 | CG2 | ILE | A | 416 | 28.071 | 34.767 | 70.578 | 1.00 | 22.00 | C |
| ATOM | 731 | CD1 | ILE | A | 416 | 30.097 | 34.308 | 68.133 | 1.00 | 22.63 | C |
| ATOM | 732 | N | ASP | A | 417 | 25.836 | 36.209 | 67.267 | 1.00 | 22.91 | N |
| ATOM | 733 | CA | ASP | A | 417 | 24.730 | 35.603 | 66.498 | 1.00 | 22.74 | C |
| ATOM | 734 | C | ASP | A | 417 | 23.358 | 36.188 | 66.862 | 1.00 | 22.69 | C |
| ATOM | 735 | O | ASP | A | 417 | 22.872 | 35.973 | 67.965 | 1.00 | 22.74 | O |
| ATOM | 736 | CB | ASP | A | 417 | 24.705 | 34.071 | 66.681 | 1.00 | 22.68 | C |
| ATOM | 737 | CG | ASP | A | 417 | 25.959 | 33.379 | 66.140 | 1.00 | 22.73 | C |
| ATOM | 738 | OD1 | ASP | A | 417 | 26.776 | 34.034 | 65.463 | 1.00 | 22.83 | O |
| ATOM | 739 | OD2 | ASP | A | 417 | 26.117 | 32.165 | 66.380 | 1.00 | 21.41 | O |
| ATOM | 740 | N | MET | A | 418 | 22.724 | 36.917 | 65.949 | 1.00 | 22.62 | N |
| ATOM | 741 | CA | MET | A | 418 | 21.377 | 37.435 | 66.238 | 1.00 | 22.84 | C |
| ATOM | 742 | C | MET | A | 418 | 20.276 | 36.870 | 65.339 | 1.00 | 22.23 | C |
| ATOM | 743 | O | MET | A | 418 | 20.518 | 36.501 | 64.196 | 1.00 | 22.31 | O |
| ATOM | 744 | CB | MET | A | 418 | 21.336 | 38.974 | 66.243 | 1.00 | 22.47 | C |
| ATOM | 745 | CG | MET | A | 418 | 21.133 | 39.628 | 64.890 | 1.00 | 23.35 | C |
| ATOM | 746 | SD | MET | A | 418 | 21.324 | 41.425 | 64.918 | 1.00 | 24.58 | S |
| ATOM | 747 | CE | MET | A | 418 | 19.665 | 41.959 | 65.158 | 1.00 | 25.04 | C |
| ATOM | 748 | N | VAL | A | 419 | 19.071 | 36.790 | 65.893 | 1.00 | 21.95 | N |
| ATOM | 749 | CA | VAL | A | 419 | 17.866 | 36.540 | 65.120 | 1.00 | 21.49 | C |
| ATOM | 750 | C | VAL | A | 419 | 16.988 | 37.791 | 65.198 | 1.00 | 21.50 | C |
| ATOM | 751 | O | VAL | A | 419 | 16.809 | 38.365 | 66.274 | 1.00 | 21.03 | O |
| ATOM | 752 | CB | VAL | A | 419 | 17.088 | 35.287 | 65.625 | 1.00 | 21.51 | C |
| ATOM | 753 | CG1 | VAL | A | 419 | 15.688 | 35.212 | 64.997 | 1.00 | 20.57 | C |
| ATOM | 754 | CG2 | VAL | A | 419 | 17.862 | 34.027 | 65.321 | 1.00 | 20.60 | C |
| ATOM | 755 | N | LEU | A | 420 | 16.473 | 38.219 | 64.047 | 1.00 | 21.40 | N |
| ATOM | 756 | CA | LEU | A | 420 | 15.499 | 39.300 | 63.986 | 1.00 | 21.44 | C |
| ATOM | 757 | C | LEU | A | 420 | 14.096 | 38.780 | 63.683 | 1.00 | 21.50 | C |
| ATOM | 758 | O | LEU | A | 420 | 13.940 | 37.803 | 62.952 | 1.00 | 21.66 | O |
| ATOM | 759 | CB | LEU | A | 420 | 15.908 | 40.329 | 62.932 | 1.00 | 21.44 | C |
| ATOM | 760 | CG | LEU | A | 420 | 17.082 | 41.224 | 63.311 | 1.00 | 20.77 | C |
| ATOM | 761 | CD1 | LEU | A | 420 | 17.558 | 42.058 | 62.119 | 1.00 | 20.39 | C |
| ATOM | 762 | CD2 | LEU | A | 420 | 16.676 | 42.103 | 64.475 | 1.00 | 20.58 | C |
| ATOM | 763 | N | VAL | A | 421 | 13.088 | 39.433 | 64.262 | 1.00 | 21.66 | N |
| ATOM | 764 | CA | VAL | A | 421 | 11.681 | 39.183 | 63.929 | 1.00 | 21.69 | C |
| ATOM | 765 | C | VAL | A | 421 | 10.826 | 40.381 | 64.343 | 1.00 | 22.17 | C |
| ATOM | 766 | O | VAL | A | 421 | 11.125 | 41.037 | 65.342 | 1.00 | 22.32 | O |
| ATOM | 767 | CB | VAL | A | 421 | 11.150 | 37.851 | 64.557 | 1.00 | 21.68 | C |
| ATOM | 768 | CG1 | VAL | A | 421 | 11.107 | 37.917 | 66.094 | 1.00 | 20.68 | C |
| ATOM | 769 | CG2 | VAL | A | 421 | 9.786 | 37.475 | 63.970 | 1.00 | 21.48 | C |
| ATOM | 770 | N | GLN | A | 422 | 9.791 | 40.687 | 63.563 | 1.00 | 22.68 | N |
| ATOM | 771 | CA | GLN | A | 422 | 8.788 | 41.666 | 63.985 | 1.00 | 23.56 | C |
| ATOM | 772 | C | GLN | A | 422 | 7.463 | 40.992 | 64.291 | 1.00 | 24.26 | C |
| ATOM | 773 | O | GLN | A | 422 | 6.983 | 40.166 | 63.516 | 1.00 | 24.71 | O |
| ATOM | 774 | CB | GLN | A | 422 | 8.572 | 42.779 | 62.948 | 1.00 | 23.41 | C |
| ATOM | 775 | CG | GLN | A | 422 | 7.658 | 43.902 | 63.472 | 1.00 | 23.31 | C |
| ATOM | 776 | CD | GLN | A | 422 | 7.566 | 45.110 | 62.561 | 1.00 | 23.70 | C |
| ATOM | 777 | OE1 | GLN | A | 422 | 7.415 | 44.985 | 61.348 | 1.00 | 25.43 | O |
| ATOM | 778 | NE2 | GLN | A | 422 | 7.646 | 46.294 | 63.150 | 1.00 | 23.70 | N |
| ATOM | 779 | N | ASP | A | 423 | 6.876 | 41.343 | 65.430 | 1.00 | 25.21 | N |
| ATOM | 780 | CA | ASP | A | 423 | 5.506 | 40.944 | 65.730 | 1.00 | 26.08 | C |
| ATOM | 781 | C | ASP | A | 423 | 4.573 | 41.827 | 64.896 | 1.00 | 26.61 | C |
| ATOM | 782 | O | ASP | A | 423 | 4.559 | 43.048 | 65.079 | 1.00 | 26.62 | O |
| ATOM | 783 | CB | ASP | A | 423 | 5.216 | 41.092 | 67.229 | 1.00 | 26.03 | C |
| ATOM | 784 | CG | ASP | A | 423 | 3.847 | 40.558 | 67.619 | 1.00 | 26.26 | C |
| ATOM | 785 | OD1 | ASP | A | 423 | 2.824 | 41.145 | 67.199 | 1.00 | 27.37 | O |
| ATOM | 786 | OD2 | ASP | A | 423 | 3.794 | 39.557 | 68.363 | 1.00 | 25.65 | O |
| ATOM | 787 | N | PRO | A | 424 | 3.791 | 41.215 | 63.979 | 1.00 | 27.09 | N |
| ATOM | 788 | CA | PRO | A | 424 | 2.919 | 41.993 | 63.084 | 1.00 | 27.35 | C |
| ATOM | 789 | C | PRO | A | 424 | 1.764 | 42.710 | 63.802 | 1.00 | 27.68 | C |
| ATOM | 790 | O | PRO | A | 424 | 1.277 | 43.723 | 63.299 | 1.00 | 27.77 | O |
| ATOM | 791 | CB | PRO | A | 424 | 2.390 | 40.944 | 62.093 | 1.00 | 27.13 | C |
| ATOM | 792 | CG | PRO | A | 424 | 2.511 | 39.654 | 62.788 | 1.00 | 26.70 | C |
| ATOM | 793 | CD | PRO | A | 424 | 3.670 | 39.764 | 63.740 | 1.00 | 26.91 | C |
| ATOM | 794 | N | GLU | A | 425 | 1.342 | 42.206 | 64.962 | 1.00 | 28.22 | N |
| ATOM | 795 | CA | GLU | A | 425 | 0.293 | 42.879 | 65.745 | 1.00 | 28.91 | C |
| ATOM | 796 | C | GLU | A | 425 | 0.847 | 44.098 | 66.467 | 1.00 | 28.65 | C |
| ATOM | 797 | O | GLU | A | 425 | 0.370 | 45.210 | 66.266 | 1.00 | 28.92 | O |
| ATOM | 798 | CB | GLU | A | 425 | −0.368 | 41.939 | 66.765 | 1.00 | 29.19 | C |
| ATOM | 799 | CG | GLU | A | 425 | −1.151 | 40.776 | 66.173 | 1.00 | 31.37 | C |
| ATOM | 800 | CD | GLU | A | 425 | −2.114 | 41.202 | 65.078 | 1.00 | 33.90 | C |
| ATOM | 801 | OE1 | GLU | A | 425 | −2.989 | 42.062 | 65.350 | 1.00 | 35.91 | O |
| ATOM | 802 | OE2 | GLU | A | 425 | −1.993 | 40.670 | 63.950 | 1.00 | 33.42 | O |
| ATOM | 803 | N | THR | A | 426 | 1.862 | 43.884 | 67.297 | 1.00 | 28.46 | N |
| ATOM | 804 | CA | THR | A | 426 | 2.388 | 44.948 | 68.154 | 1.00 | 28.45 | C |
| ATOM | 805 | C | THR | A | 426 | 3.330 | 45.900 | 67.425 | 1.00 | 27.97 | C |
| ATOM | 806 | O | THR | A | 426 | 3.564 | 47.008 | 67.889 | 1.00 | 27.99 | O |
| ATOM | 807 | CB | THR | A | 426 | 3.102 | 44.369 | 69.388 | 1.00 | 28.38 | C |
| ATOM | 808 | OG1 | THR | A | 426 | 2.326 | 43.291 | 69.917 | 1.00 | 28.44 | O |
| ATOM | 809 | CG2 | THR | A | 426 | 3.253 | 45.436 | 70.472 | 1.00 | 29.45 | C |
| ATOM | 810 | N | LYS | A | 427 | 3.851 | 45.461 | 66.280 | 1.00 | 27.76 | N |
| ATOM | 811 | CA | LYS | A | 427 | 4.873 | 46.191 | 65.511 | 1.00 | 27.33 | C |
| ATOM | 812 | C | LYS | A | 427 | 6.201 | 46.256 | 66.274 | 1.00 | 26.88 | C |
| ATOM | 813 | O | LYS | A | 427 | 7.060 | 47.106 | 65.990 | 1.00 | 26.96 | O |
| ATOM | 814 | CB | LYS | A | 427 | 4.399 | 47.600 | 65.088 | 1.00 | 27.67 | C |
| ATOM | 815 | CG | LYS | A | 427 | 2.959 | 47.695 | 64.538 | 1.00 | 28.12 | C |
| ATOM | 816 | CD | LYS | A | 427 | 2.722 | 46.795 | 63.320 | 1.00 | 28.71 | C |
| ATOM | 817 | CE | LYS | A | 427 | 1.463 | 47.191 | 62.540 | 1.00 | 29.13 | C |
| ATOM | 818 | NZ | LYS | A | 427 | 0.209 | 47.155 | 63.350 | 1.00 | 29.68 | N |
| ATOM | 819 | N | ARG | A | 428 | 6.366 | 45.356 | 67.242 | 1.00 | 25.78 | N |
| ATOM | 820 | CA | ARG | A | 428 | 7.591 | 45.300 | 68.019 | 1.00 | 24.94 | C |
| ATOM | 821 | C | ARG | A | 428 | 8.640 | 44.432 | 67.323 | 1.00 | 24.48 | C |
| ATOM | 822 | O | ARG | A | 428 | 8.339 | 43.338 | 66.849 | 1.00 | 24.34 | O |
| ATOM | 823 | CB | ARG | A | 428 | 7.320 | 44.803 | 69.437 | 1.00 | 24.89 | C |
| ATOM | 824 | CG | ARG | A | 428 | 8.563 | 44.740 | 70.312 | 1.00 | 24.28 | C |
| ATOM | 825 | CD | ARG | A | 428 | 8.206 | 44.533 | 71.765 | 1.00 | 24.09 | C |
| ATOM | 826 | NE | ARG | A | 428 | 7.825 | 45.783 | 72.410 | 1.00 | 23.84 | N |
| ATOM | 827 | CZ | ARG | A | 428 | 7.575 | 45.910 | 73.707 | 1.00 | 24.39 | C |
| ATOM | 828 | NH1 | ARG | A | 428 | 7.657 | 44.854 | 74.510 | 1.00 | 24.65 | N |
| ATOM | 829 | NH2 | ARG | A | 428 | 7.243 | 47.096 | 74.201 | 1.00 | 24.36 | N |
| ATOM | 830 | N | ILE | A | 429 | 9.866 | 44.944 | 67.268 | 1.00 | 23.80 | N |
| ATOM | 831 | CA | ILE | A | 429 | 10.972 | 44.250 | 66.633 | 1.00 | 23.40 | C |
| ATOM | 832 | C | ILE | A | 429 | 11.899 | 43.669 | 67.689 | 1.00 | 23.09 | C |
| ATOM | 833 | O | ILE | A | 429 | 12.382 | 44.379 | 68.572 | 1.00 | 22.96 | O |
| ATOM | 834 | CB | ILE | A | 429 | 11.755 | 45.181 | 65.667 | 1.00 | 23.39 | C |
| ATOM | 835 | CG1 | ILE | A | 429 | 10.875 | 45.555 | 64.468 | 1.00 | 23.36 | C |
| ATOM | 836 | CG2 | ILE | A | 429 | 13.033 | 44.506 | 65.187 | 1.00 | 22.89 | C |
| ATOM | 837 | CD1 | ILE | A | 429 | 11.206 | 46.894 | 63.845 | 1.00 | 23.02 | C |
| ATOM | 838 | N | PHE | A | 430 | 12.138 | 42.368 | 67.582 | 1.00 | 22.75 | N |
| ATOM | 839 | CA | PHE | A | 430 | 12.982 | 41.655 | 68.523 | 1.00 | 22.29 | C |
| ATOM | 840 | C | PHE | A | 430 | 14.305 | 41.279 | 67.891 | 1.00 | 22.18 | C |
| ATOM | 841 | O | PHE | A | 430 | 14.356 | 40.834 | 66.743 | 1.00 | 22.04 | O |
| ATOM | 842 | CB | PHE | A | 430 | 12.289 | 40.380 | 69.002 | 1.00 | 22.18 | C |
| ATOM | 843 | CG | PHE | A | 430 | 10.974 | 40.621 | 69.670 | 1.00 | 22.06 | C |
| ATOM | 844 | CD1 | PHE | A | 430 | 10.910 | 40.861 | 71.038 | 1.00 | 22.00 | C |
| ATOM | 845 | CD2 | PHE | A | 430 | 9.796 | 40.599 | 68.935 | 1.00 | 21.34 | C |
| ATOM | 846 | CE1 | PHE | A | 430 | 9.683 | 41.088 | 71.668 | 1.00 | 22.14 | C |
| ATOM | 847 | CE2 | PHE | A | 430 | 8.569 | 40.823 | 69.554 | 1.00 | 22.24 | C |
| ATOM | 848 | CZ | PHE | A | 430 | 8.513 | 41.067 | 70.926 | 1.00 | 21.79 | C |
| ATOM | 849 | N | SER | A | 431 | 15.374 | 41.466 | 68.654 | 1.00 | 21.99 | N |
| ATOM | 850 | CA | SER | A | 431 | 16.661 | 40.883 | 68.325 | 1.00 | 21.70 | C |
| ATOM | 851 | C | SER | A | 431 | 17.085 | 39.958 | 69.462 | 1.00 | 21.43 | C |
| ATOM | 852 | O | SER | A | 431 | 17.281 | 40.393 | 70.591 | 1.00 | 21.70 | O |
| ATOM | 853 | CB | SER | A | 431 | 17.705 | 41.970 | 68.092 | 1.00 | 21.69 | C |
| ATOM | 854 | OG | SER | A | 431 | 19.001 | 41.404 | 67.999 | 1.00 | 21.91 | O |
| ATOM | 855 | N | ILE | A | 432 | 17.204 | 38.677 | 69.156 | 1.00 | 21.28 | N |
| ATOM | 856 | CA | ILE | A | 432 | 17.612 | 37.680 | 70.139 | 1.00 | 21.08 | C |
| ATOM | 857 | C | ILE | A | 432 | 18.994 | 37.153 | 69.760 | 1.00 | 20.93 | C |
| ATOM | 858 | O | ILE | A | 432 | 19.267 | 36.880 | 68.589 | 1.00 | 20.68 | O |
| ATOM | 859 | CB | ILE | A | 432 | 16.553 | 36.555 | 70.273 | 1.00 | 21.11 | C |
| ATOM | 860 | CG1 | ILE | A | 432 | 15.202 | 37.178 | 70.651 | 1.00 | 21.17 | C |
| ATOM | 861 | CG2 | ILE | A | 432 | 16.988 | 35.509 | 71.306 | 1.00 | 20.95 | C |
| ATOM | 862 | CD1 | ILE | A | 432 | 14.020 | 36.256 | 70.594 | 1.00 | 21.89 | C |
| ATOM | 863 | N | TYR | A | 433 | 19.871 | 37.057 | 70.753 | 1.00 | 20.88 | N |
| ATOM | 864 | CA | TYR | A | 433 | 21.285 | 36.771 | 70.518 | 1.00 | 20.89 | C |
| ATOM | 865 | C | TYR | A | 433 | 21.920 | 36.111 | 71.727 | 1.00 | 20.88 | C |
| ATOM | 866 | O | TYR | A | 433 | 21.401 | 36.201 | 72.838 | 1.00 | 20.75 | O |
| ATOM | 867 | CB | TYR | A | 433 | 22.041 | 38.055 | 70.155 | 1.00 | 21.09 | C |
| ATOM | 868 | CG | TYR | A | 433 | 21.738 | 39.214 | 71.072 | 1.00 | 21.23 | C |
| ATOM | 869 | CD1 | TYR | A | 433 | 20.721 | 40.117 | 70.771 | 1.00 | 21.42 | C |
| ATOM | 870 | CD2 | TYR | A | 433 | 22.459 | 39.403 | 72.249 | 1.00 | 21.70 | C |
| ATOM | 871 | CE1 | TYR | A | 433 | 20.425 | 41.180 | 71.620 | 1.00 | 21.58 | C |
| ATOM | 872 | CE2 | TYR | A | 433 | 22.169 | 40.465 | 73.106 | 1.00 | 22.32 | C |
| ATOM | 873 | CZ | TYR | A | 433 | 21.149 | 41.343 | 72.783 | 1.00 | 21.42 | C |
| ATOM | 874 | OH | TYR | A | 433 | 20.858 | 42.387 | 73.624 | 1.00 | 22.09 | O |
| ATOM | 875 | N | ASP | A | 434 | 23.046 | 35.446 | 71.500 | 1.00 | 21.07 | N |
| ATOM | 876 | CA | ASP | A | 434 | 23.740 | 34.712 | 72.549 | 1.00 | 21.21 | C |
| ATOM | 877 | C | ASP | A | 434 | 24.471 | 35.661 | 73.489 | 1.00 | 21.46 | C |
| ATOM | 878 | O | ASP | A | 434 | 24.738 | 36.807 | 73.142 | 1.00 | 21.64 | O |
| ATOM | 879 | CB | ASP | A | 434 | 24.745 | 33.730 | 71.940 | 1.00 | 21.08 | C |
| ATOM | 880 | CG | ASP | A | 434 | 24.112 | 32.765 | 70.964 | 1.00 | 20.98 | C |
| ATOM | 881 | OD1 | ASP | A | 434 | 22.874 | 32.783 | 70.816 | 1.00 | 20.98 | O |
| ATOM | 882 | OD2 | ASP | A | 434 | 24.857 | 31.979 | 70.340 | 1.00 | 21.05 | O |
| ATOM | 883 | N | MET | A | 435 | 24.791 | 35.168 | 74.678 | 1.00 | 21.97 | N |
| ATOM | 884 | CA | MET | A | 435 | 25.652 | 35.883 | 75.606 | 1.00 | 22.48 | C |
| ATOM | 885 | C | MET | A | 435 | 26.574 | 34.912 | 76.352 | 1.00 | 23.01 | C |
| ATOM | 886 | O | MET | A | 435 | 26.131 | 33.881 | 76.874 | 1.00 | 22.98 | O |
| ATOM | 887 | CB | MET | A | 435 | 24.829 | 36.738 | 76.580 | 1.00 | 22.40 | C |
| ATOM | 888 | CG | MET | A | 435 | 25.663 | 37.680 | 77.450 | 1.00 | 22.17 | C |
| ATOM | 889 | SD | MET | A | 435 | 26.263 | 36.904 | 78.973 | 1.00 | 22.48 | S |
| ATOM | 890 | CE | MET | A | 435 | 27.561 | 38.050 | 79.457 | 1.00 | 22.12 | C |
| ATOM | 891 | N | PHE | A | 436 | 27.861 | 35.246 | 76.359 | 1.00 | 23.51 | N |
| ATOM | 892 | CA | PHE | A | 436 | 28.872 | 34.555 | 77.162 | 1.00 | 24.26 | C |
| ATOM | 893 | C | PHE | A | 436 | 29.616 | 35.626 | 77.939 | 1.00 | 24.65 | C |
| ATOM | 894 | O | PHE | A | 436 | 29.858 | 36.712 | 77.405 | 1.00 | 25.02 | O |
| ATOM | 895 | CB | PHE | A | 436 | 29.901 | 33.830 | 76.280 | 1.00 | 24.06 | C |
| ATOM | 896 | CG | PHE | A | 436 | 29.343 | 32.688 | 75.485 | 1.00 | 24.12 | C |
| ATOM | 897 | CD1 | PHE | A | 436 | 29.376 | 31.390 | 75.993 | 1.00 | 23.57 | C |
| ATOM | 898 | CD2 | PHE | A | 436 | 28.815 | 32.899 | 74.209 | 1.00 | 23.69 | C |
| ATOM | 899 | CE1 | PHE | A | 436 | 28.870 | 30.322 | 75.255 | 1.00 | 23.42 | C |
| ATOM | 900 | CE2 | PHE | A | 436 | 28.310 | 31.833 | 73.462 | 1.00 | 23.61 | C |
| ATOM | 901 | CZ | PHE | A | 436 | 28.339 | 30.540 | 73.989 | 1.00 | 23.28 | C |
| ATOM | 902 | N | PRO | A | 437 | 29.984 | 35.337 | 79.200 | 1.00 | 25.06 | N |
| ATOM | 903 | CA | PRO | A | 437 | 30.945 | 36.214 | 79.868 | 1.00 | 25.30 | C |
| ATOM | 904 | C | PRO | A | 437 | 32.326 | 36.107 | 79.213 | 1.00 | 25.67 | C |
| ATOM | 905 | O | PRO | A | 437 | 32.504 | 35.321 | 78.271 | 1.00 | 25.50 | O |
| ATOM | 906 | CB | PRO | A | 437 | 30.971 | 35.678 | 81.305 | 1.00 | 25.32 | C |
| ATOM | 907 | CG | PRO | A | 437 | 30.510 | 34.267 | 81.209 | 1.00 | 25.01 | C |
| ATOM | 908 | CD | PRO | A | 437 | 29.527 | 34.238 | 80.074 | 1.00 | 25.10 | C |
| ATOM | 909 | N | GLU | A | 438 | 33.280 | 36.899 | 79.703 | 1.00 | 26.34 | N |
| ATOM | 910 | CA | GLU | A | 438 | 34.667 | 36.892 | 79.213 | 1.00 | 26.89 | C |
| ATOM | 911 | C | GLU | A | 438 | 35.171 | 35.463 | 78.993 | 1.00 | 27.13 | C |
| ATOM | 912 | O | GLU | A | 438 | 35.102 | 34.628 | 79.897 | 1.00 | 27.06 | O |
| ATOM | 913 | CB | GLU | A | 438 | 35.580 | 37.654 | 80.198 | 1.00 | 27.05 | C |
| ATOM | 914 | CG | GLU | A | 438 | 37.092 | 37.651 | 79.870 | 1.00 | 27.00 | C |
| ATOM | 915 | CD | GLU | A | 438 | 37.968 | 38.247 | 80.985 | 1.00 | 27.15 | C |
| ATOM | 916 | OE1 | GLU | A | 438 | 37.438 | 38.657 | 82.043 | 1.00 | 27.17 | O |
| ATOM | 917 | OE2 | GLU | A | 438 | 39.200 | 38.305 | 80.800 | 1.00 | 27.28 | O |
| ATOM | 918 | N | GLY | A | 439 | 35.663 | 35.190 | 77.786 | 1.00 | 27.38 | N |
| ATOM | 919 | CA | GLY | A | 439 | 36.186 | 33.869 | 77.437 | 1.00 | 27.69 | C |
| ATOM | 920 | C | GLY | A | 439 | 36.187 | 33.655 | 75.937 | 1.00 | 28.01 | C |
| ATOM | 921 | O | GLY | A | 439 | 36.276 | 34.618 | 75.175 | 1.00 | 27.99 | O |
| ATOM | 922 | N | LYS | A | 440 | 36.070 | 32.398 | 75.515 | 1.00 | 28.29 | N |
| ATOM | 923 | CA | LYS | A | 440 | 36.070 | 32.055 | 74.091 | 1.00 | 28.86 | C |
| ATOM | 924 | C | LYS | A | 440 | 34.708 | 31.562 | 73.572 | 1.00 | 28.91 | C |
| ATOM | 925 | O | LYS | A | 440 | 34.643 | 30.751 | 72.646 | 1.00 | 28.81 | O |
| ATOM | 926 | CB | LYS | A | 440 | 37.189 | 31.056 | 73.771 | 1.00 | 28.79 | C |
| ATOM | 927 | CG | LYS | A | 440 | 38.573 | 31.669 | 73.788 | 1.00 | 29.25 | C |
| ATOM | 928 | CD | LYS | A | 440 | 39.671 | 30.647 | 73.514 | 1.00 | 29.58 | C |
| ATOM | 929 | CE | LYS | A | 440 | 41.025 | 31.181 | 73.981 | 1.00 | 32.26 | C |
| ATOM | 930 | NZ | LYS | A | 440 | 42.210 | 30.475 | 73.387 | 1.00 | 34.61 | N |
| ATOM | 931 | N | GLY | A | 441 | 33.635 | 32.066 | 74.182 | 1.00 | 29.22 | N |
| ATOM | 932 | CA | GLY | A | 441 | 32.247 | 31.837 | 73.751 | 1.00 | 29.39 | C |
| ATOM | 933 | C | GLY | A | 441 | 31.902 | 30.624 | 72.898 | 1.00 | 29.74 | C |
| ATOM | 934 | O | GLY | A | 441 | 31.804 | 30.723 | 71.664 | 1.00 | 30.16 | O |
| ATOM | 935 | N | ILE | A | 442 | 31.692 | 29.495 | 73.570 | 1.00 | 29.48 | N |
| ATOM | 936 | CA | ILE | A | 442 | 31.331 | 28.183 | 72.982 | 1.00 | 29.24 | C |
| ATOM | 937 | C | ILE | A | 442 | 32.529 | 27.260 | 72.835 | 1.00 | 29.30 | C |
| ATOM | 938 | O | ILE | A | 442 | 32.421 | 26.060 | 73.087 | 1.00 | 29.36 | O |
| ATOM | 939 | CB | ILE | A | 442 | 30.471 | 28.218 | 71.676 | 1.00 | 29.23 | C |
| ATOM | 940 | CG1 | ILE | A | 442 | 29.521 | 27.013 | 71.663 | 1.00 | 28.90 | C |
| ATOM | 941 | CG2 | ILE | A | 442 | 31.342 | 28.223 | 70.406 | 1.00 | 29.20 | C |
| ATOM | 942 | CD1 | ILE | A | 442 | 28.568 | 26.969 | 70.500 | 1.00 | 28.87 | C |
| ATOM | 943 | N | PHE | A | 443 | 33.666 | 27.827 | 72.441 | 1.00 | 29.29 | N |
| ATOM | 944 | CA | PHE | A | 443 | 34.917 | 27.082 | 72.423 | 1.00 | 29.35 | C |
| ATOM | 945 | C | PHE | A | 443 | 35.681 | 27.286 | 73.730 | 1.00 | 29.62 | C |
| ATOM | 946 | O | PHE | A | 443 | 36.812 | 26.820 | 73.869 | 1.00 | 29.85 | O |
| ATOM | 947 | CB | PHE | A | 443 | 35.779 | 27.487 | 71.226 | 1.00 | 29.12 | C |
| ATOM | 948 | CG | PHE | A | 443 | 35.159 | 27.170 | 69.898 | 1.00 | 28.46 | C |
| ATOM | 949 | CD1 | PHE | A | 443 | 35.135 | 25.864 | 69.421 | 1.00 | 27.52 | C |
| ATOM | 950 | CD2 | PHE | A | 443 | 34.608 | 28.183 | 69.117 | 1.00 | 28.14 | C |
| ATOM | 951 | CE1 | PHE | A | 443 | 34.567 | 25.569 | 68.190 | 1.00 | 27.35 | C |
| ATOM | 952 | CE2 | PHE | A | 443 | 34.038 | 27.897 | 67.882 | 1.00 | 28.03 | C |
| ATOM | 953 | CZ | PHE | A | 443 | 34.018 | 26.585 | 67.418 | 1.00 | 27.93 | C |
| ATOM | 954 | N | GLY | A | 444 | 35.051 | 27.974 | 74.682 | 1.00 | 29.88 | N |
| ATOM | 955 | CA | GLY | A | 444 | 35.673 | 28.271 | 75.969 | 1.00 | 30.23 | C |
| ATOM | 956 | C | GLY | A | 444 | 34.792 | 27.965 | 77.165 | 1.00 | 30.47 | C |
| ATOM | 957 | O | GLY | A | 444 | 34.915 | 28.599 | 78.215 | 1.00 | 30.75 | O |
| ATOM | 958 | N | MET | A | 445 | 33.903 | 26.993 | 77.008 | 1.00 | 30.69 | N |
| ATOM | 959 | CA | MET | A | 445 | 32.998 | 26.609 | 78.080 | 1.00 | 30.93 | C |
| ATOM | 960 | C | MET | A | 445 | 33.650 | 25.617 | 79.045 | 1.00 | 31.23 | C |
| ATOM | 961 | O | MET | A | 445 | 34.426 | 24.744 | 78.636 | 1.00 | 31.25 | O |
| ATOM | 962 | CB | MET | A | 445 | 31.713 | 26.021 | 77.505 | 1.00 | 30.86 | C |
| ATOM | 963 | CG | MET | A | 445 | 30.912 | 26.994 | 76.638 | 1.00 | 31.34 | C |
| ATOM | 964 | SD | MET | A | 445 | 29.299 | 26.331 | 76.177 | 1.00 | 31.05 | S |
| ATOM | 965 | CE | MET | A | 445 | 28.417 | 26.465 | 77.738 | 1.00 | 32.17 | C |
| ATOM | 966 | N | SER | A | 446 | 33.322 | 25.766 | 80.327 | 1.00 | 31.49 | N |
| ATOM | 967 | CA | SER | A | 446 | 33.792 | 24.870 | 81.376 | 1.00 | 31.67 | C |
| ATOM | 968 | C | SER | A | 446 | 33.178 | 23.492 | 81.197 | 1.00 | 31.55 | C |
| ATOM | 969 | O | SER | A | 446 | 32.053 | 23.370 | 80.706 | 1.00 | 31.82 | O |
| ATOM | 970 | CB | SER | A | 446 | 33.415 | 25.426 | 82.750 | 1.00 | 31.84 | C |
| ATOM | 971 | OG | SER | A | 446 | 33.575 | 24.444 | 83.765 | 1.00 | 32.86 | O |
| ATOM | 972 | N | SER | A | 447 | 33.917 | 22.459 | 81.597 | 1.00 | 31.13 | N |
| ATOM | 973 | CA | SER | A | 447 | 33.421 | 21.090 | 81.501 | 1.00 | 30.68 | C |
| ATOM | 974 | C | SER | A | 447 | 32.562 | 20.718 | 82.710 | 1.00 | 30.22 | C |
| ATOM | 975 | O | SER | A | 447 | 31.979 | 19.638 | 82.746 | 1.00 | 30.35 | O |
| ATOM | 976 | CB | SER | A | 447 | 34.579 | 20.100 | 81.334 | 1.00 | 30.68 | C |
| ATOM | 977 | OG | SER | A | 447 | 35.318 | 19.974 | 82.535 | 1.00 | 31.08 | O |
| ATOM | 978 | N | GLN | A | 448 | 32.502 | 21.607 | 83.700 | 1.00 | 29.73 | N |
| ATOM | 979 | CA | GLN | A | 448 | 31.628 | 21.417 | 84.863 | 1.00 | 29.21 | C |
| ATOM | 980 | C | GLN | A | 448 | 30.566 | 22.515 | 84.954 | 1.00 | 29.04 | C |
| ATOM | 981 | O | GLN | A | 448 | 30.813 | 23.670 | 84.591 | 1.00 | 29.02 | O |
| ATOM | 982 | CB | GLN | A | 448 | 32.431 | 21.351 | 86.164 | 1.00 | 29.09 | C |
| ATOM | 983 | CG | GLN | A | 448 | 33.414 | 20.180 | 86.264 | 1.00 | 28.72 | C |
| ATOM | 984 | CD | GLN | A | 448 | 32.736 | 18.822 | 86.323 | 1.00 | 27.71 | C |
| ATOM | 985 | OE1 | GLN | A | 448 | 31.738 | 18.635 | 87.015 | 1.00 | 27.04 | O |
| ATOM | 986 | NE2 | GLN | A | 448 | 33.288 | 17.864 | 85.598 | 1.00 | 27.56 | N |
| ATOM | 987 | N | LYS | A | 449 | 29.386 | 22.140 | 85.437 | 1.00 | 28.75 | N |
| ATOM | 988 | CA | LYS | A | 449 | 28.260 | 23.056 | 85.547 | 1.00 | 28.13 | C |
| ATOM | 989 | C | LYS | A | 449 | 28.364 | 23.905 | 86.803 | 1.00 | 28.03 | C |
| ATOM | 990 | O | LYS | A | 449 | 28.642 | 23.401 | 87.890 | 1.00 | 27.89 | O |
| ATOM | 991 | CB | LYS | A | 449 | 26.935 | 22.281 | 85.551 | 1.00 | 28.13 | C |
| ATOM | 992 | CG | LYS | A | 449 | 25.683 | 23.146 | 85.666 | 1.00 | 27.71 | C |
| ATOM | 993 | CD | LYS | A | 449 | 24.433 | 22.287 | 85.687 | 1.00 | 27.89 | C |
| ATOM | 994 | CE | LYS | A | 449 | 23.158 | 23.122 | 85.668 | 1.00 | 27.72 | C |
| ATOM | 995 | NZ | LYS | A | 449 | 22.887 | 23.694 | 84.328 | 1.00 | 27.00 | N |
| ATOM | 996 | N | GLU | A | 450 | 28.149 | 25.203 | 86.631 | 1.00 | 27.87 | N |
| ATOM | 997 | CA | GLU | A | 450 | 27.903 | 26.104 | 87.740 | 1.00 | 27.86 | C |
| ATOM | 998 | C | GLU | A | 450 | 26.457 | 26.551 | 87.580 | 1.00 | 27.60 | C |
| ATOM | 999 | O | GLU | A | 450 | 26.071 | 26.999 | 86.508 | 1.00 | 27.70 | O |
| ATOM | 1000 | CB | GLU | A | 450 | 28.853 | 27.312 | 87.693 | 1.00 | 27.98 | C |
| ATOM | 1001 | CG | GLU | A | 450 | 30.340 | 26.969 | 87.573 | 1.00 | 27.79 | C |
| ATOM | 1002 | CD | GLU | A | 450 | 31.243 | 28.200 | 87.508 | 1.00 | 28.15 | C |
| ATOM | 1003 | OE1 | GLU | A | 450 | 30.733 | 29.320 | 87.274 | 1.00 | 27.29 | O |
| ATOM | 1004 | OE2 | GLU | A | 450 | 32.475 | 28.042 | 87.693 | 1.00 | 28.47 | O |
| ATOM | 1005 | N | GLU | A | 451 | 25.653 | 26.404 | 88.629 | 1.00 | 27.58 | N |
| ATOM | 1006 | CA | GLU | A | 451 | 24.268 | 26.881 | 88.609 | 1.00 | 27.46 | C |
| ATOM | 1007 | C | GLU | A | 451 | 24.236 | 28.372 | 88.260 | 1.00 | 27.42 | C |
| ATOM | 1008 | O | GLU | A | 451 | 24.937 | 29.177 | 88.885 | 1.00 | 27.25 | O |
| ATOM | 1009 | CB | GLU | A | 451 | 23.593 | 26.656 | 89.968 | 1.00 | 27.50 | C |
| ATOM | 1010 | CG | GLU | A | 451 | 23.557 | 25.206 | 90.466 | 1.00 | 28.34 | C |
| ATOM | 1011 | CD | GLU | A | 451 | 22.642 | 24.295 | 89.650 | 1.00 | 30.83 | C |
| ATOM | 1012 | OE1 | GLU | A | 451 | 21.708 | 24.795 | 88.967 | 1.00 | 31.10 | O |
| ATOM | 1013 | OE2 | GLU | A | 451 | 22.861 | 23.061 | 89.698 | 1.00 | 31.27 | O |
| ATOM | 1014 | N | ALA | A | 452 | 23.440 | 28.740 | 87.258 | 1.00 | 27.16 | N |
| ATOM | 1015 | CA | ALA | A | 452 | 23.347 | 30.144 | 86.857 | 1.00 | 27.28 | C |
| ATOM | 1016 | C | ALA | A | 452 | 22.192 | 30.905 | 87.520 | 1.00 | 27.29 | C |
| ATOM | 1017 | O | ALA | A | 452 | 22.262 | 32.121 | 87.680 | 1.00 | 27.18 | O |
| ATOM | 1018 | CB | ALA | A | 452 | 23.297 | 30.273 | 85.332 | 1.00 | 27.02 | C |
| ATOM | 1019 | N | TYR | A | 453 | 21.147 | 30.181 | 87.913 | 1.00 | 27.68 | N |
| ATOM | 1020 | CA | TYR | A | 453 | 19.911 | 30.785 | 88.422 | 1.00 | 28.01 | C |
| ATOM | 1021 | C | TYR | A | 453 | 19.380 | 30.015 | 89.623 | 1.00 | 28.98 | C |
| ATOM | 1022 | O | TYR | A | 453 | 19.600 | 28.803 | 89.746 | 1.00 | 29.02 | O |
| ATOM | 1023 | CB | TYR | A | 453 | 18.831 | 30.840 | 87.324 | 1.00 | 27.18 | C |
| ATOM | 1024 | CG | TYR | A | 453 | 19.238 | 31.623 | 86.092 | 1.00 | 26.04 | C |
| ATOM | 1025 | CD1 | TYR | A | 453 | 19.073 | 33.010 | 86.032 | 1.00 | 24.87 | C |
| ATOM | 1026 | CD2 | TYR | A | 453 | 19.791 | 30.976 | 84.989 | 1.00 | 24.60 | C |
| ATOM | 1027 | CE1 | TYR | A | 453 | 19.456 | 33.728 | 84.906 | 1.00 | 24.60 | C |
| ATOM | 1028 | CE2 | TYR | A | 453 | 20.176 | 31.680 | 83.862 | 1.00 | 23.78 | C |
| ATOM | 1029 | CZ | TYR | A | 453 | 20.011 | 33.051 | 83.826 | 1.00 | 24.97 | C |
| ATOM | 1030 | OH | TYR | A | 453 | 20.402 | 33.740 | 82.706 | 1.00 | 25.06 | O |
| ATOM | 1031 | N | LYS | A | 454 | 18.678 | 30.726 | 90.504 | 1.00 | 30.08 | N |
| ATOM | 1032 | CA | LYS | A | 454 | 18.076 | 30.121 | 91.689 | 1.00 | 31.15 | C |
| ATOM | 1033 | C | LYS | A | 454 | 16.626 | 30.589 | 91.862 | 1.00 | 31.82 | C |
| ATOM | 1034 | O | LYS | A | 454 | 16.318 | 31.760 | 91.623 | 1.00 | 31.94 | O |
| ATOM | 1035 | CB | LYS | A | 454 | 18.909 | 30.455 | 92.934 | 1.00 | 31.33 | C |
| ATOM | 1036 | CG | LYS | A | 454 | 18.583 | 29.601 | 94.152 | 1.00 | 31.98 | C |
| ATOM | 1037 | CD | LYS | A | 454 | 19.606 | 29.749 | 95.254 | 1.00 | 32.85 | C |
| ATOM | 1038 | CE | LYS | A | 454 | 19.384 | 28.677 | 96.311 | 1.00 | 34.21 | C |
| ATOM | 1039 | NZ | LYS | A | 454 | 20.418 | 28.710 | 97.385 | 1.00 | 35.38 | N |
| ATOM | 1040 | N | LYS | A | 455 | 15.743 | 29.672 | 92.266 | 1.00 | 32.56 | N |
| ATOM | 1041 | CA | LYS | A | 455 | 14.345 | 30.012 | 92.541 | 1.00 | 33.36 | C |
| ATOM | 1042 | C | LYS | A | 455 | 14.082 | 30.168 | 94.045 | 1.00 | 33.80 | C |
| ATOM | 1043 | O | LYS | A | 455 | 13.848 | 29.182 | 94.758 | 1.00 | 33.99 | O |
| ATOM | 1044 | CB | LYS | A | 455 | 13.392 | 28.981 | 91.918 | 1.00 | 33.32 | C |
| ATOM | 1045 | CG | LYS | A | 455 | 11.947 | 29.478 | 91.778 | 1.00 | 33.38 | C |
| ATOM | 1046 | CD | LYS | A | 455 | 11.103 | 28.551 | 90.915 | 1.00 | 33.73 | C |
| ATOM | 1047 | CE | LYS | A | 455 | 11.440 | 28.708 | 89.431 | 1.00 | 35.53 | C |
| ATOM | 1048 | NZ | LYS | A | 455 | 10.645 | 27.794 | 88.554 | 1.00 | 35.83 | N |
| ATOM | 1049 | N | ILE | A | 456 | 14.132 | 31.413 | 94.518 | 1.00 | 34.33 | N |
| ATOM | 1050 | CA | ILE | A | 456 | 13.875 | 31.723 | 95.921 | 1.00 | 34.64 | C |
| ATOM | 1051 | C | ILE | A | 456 | 12.438 | 32.204 | 96.078 | 1.00 | 35.06 | C |
| ATOM | 1052 | O | ILE | A | 456 | 12.077 | 33.286 | 95.596 | 1.00 | 35.06 | O |
| ATOM | 1053 | CB | ILE | A | 456 | 14.884 | 32.764 | 96.483 | 1.00 | 34.86 | C |
| ATOM | 1054 | CG1 | ILE | A | 456 | 16.275 | 32.128 | 96.613 | 1.00 | 34.65 | C |
| ATOM | 1055 | CG2 | ILE | A | 456 | 14.419 | 33.311 | 97.846 | 1.00 | 34.84 | C |
| ATOM | 1056 | CD1 | ILE | A | 456 | 17.398 | 33.111 | 96.824 | 1.00 | 33.67 | C |
| ATOM | 1057 | N | ASP | A | 457 | 11.624 | 31.373 | 96.734 | 1.00 | 35.43 | N |
| ATOM | 1058 | CA | ASP | A | 457 | 10.211 | 31.667 | 96.999 | 1.00 | 35.68 | C |
| ATOM | 1059 | C | ASP | A | 457 | 9.441 | 31.994 | 95.714 | 1.00 | 35.54 | C |
| ATOM | 1060 | O | ASP | A | 457 | 8.719 | 32.997 | 95.634 | 1.00 | 35.36 | O |
| ATOM | 1061 | CB | ASP | A | 457 | 10.075 | 32.796 | 98.036 | 1.00 | 36.00 | C |
| ATOM | 1062 | CG | ASP | A | 457 | 8.673 | 32.903 | 98.611 | 1.00 | 37.06 | C |
| ATOM | 1063 | OD1 | ASP | A | 457 | 8.129 | 31.869 | 99.069 | 1.00 | 38.08 | O |
| ATOM | 1064 | OD2 | ASP | A | 457 | 8.119 | 34.027 | 98.609 | 1.00 | 37.98 | O |
| ATOM | 1065 | N | GLY | A | 458 | 9.615 | 31.140 | 94.708 | 1.00 | 35.45 | N |
| ATOM | 1066 | CA | GLY | A | 458 | 8.890 | 31.266 | 93.444 | 1.00 | 35.31 | C |
| ATOM | 1067 | C | GLY | A | 458 | 9.391 | 32.320 | 92.467 | 1.00 | 35.17 | C |
| ATOM | 1068 | O | GLY | A | 458 | 8.835 | 32.461 | 91.379 | 1.00 | 35.36 | O |
| ATOM | 1069 | N | LYS | A | 459 | 10.426 | 33.065 | 92.844 | 1.00 | 34.83 | N |
| ATOM | 1070 | CA | LYS | A | 459 | 11.026 | 34.048 | 91.943 | 1.00 | 34.52 | C |
| ATOM | 1071 | C | LYS | A | 459 | 12.431 | 33.620 | 91.551 | 1.00 | 34.05 | C |
| ATOM | 1072 | O | LYS | A | 459 | 13.197 | 33.146 | 92.396 | 1.00 | 34.34 | O |
| ATOM | 1073 | CB | LYS | A | 459 | 11.059 | 35.444 | 92.575 | 1.00 | 34.73 | C |
| ATOM | 1074 | CG | LYS | A | 459 | 9.725 | 36.175 | 92.571 | 1.00 | 35.27 | C |
| ATOM | 1075 | CD | LYS | A | 459 | 9.925 | 37.690 | 92.527 | 1.00 | 36.81 | C |
| ATOM | 1076 | CE | LYS | A | 459 | 8.635 | 38.434 | 92.884 | 1.00 | 37.56 | C |
| ATOM | 1077 | NZ | LYS | A | 459 | 8.645 | 39.850 | 92.410 | 1.00 | 37.55 | N |
| ATOM | 1078 | N | THR | A | 460 | 12.758 | 33.797 | 90.271 | 1.00 | 33.21 | N |
| ATOM | 1079 | CA | THR | A | 460 | 14.049 | 33.402 | 89.715 | 1.00 | 32.58 | C |
| ATOM | 1080 | C | THR | A | 460 | 15.046 | 34.570 | 89.731 | 1.00 | 32.18 | C |
| ATOM | 1081 | O | THR | A | 460 | 14.779 | 35.640 | 89.180 | 1.00 | 32.07 | O |
| ATOM | 1082 | CB | THR | A | 460 | 13.886 | 32.834 | 88.267 | 1.00 | 32.65 | C |
| ATOM | 1083 | OG1 | THR | A | 460 | 13.015 | 31.695 | 88.291 | 1.00 | 32.79 | O |
| ATOM | 1084 | CG2 | THR | A | 460 | 15.228 | 32.410 | 87.673 | 1.00 | 32.25 | C |
| ATOM | 1085 | N | TYR | A | 461 | 16.190 | 34.362 | 90.377 | 1.00 | 31.76 | N |
| ATOM | 1086 | CA | TYR | A | 461 | 17.275 | 35.352 | 90.366 | 1.00 | 31.46 | C |
| ATOM | 1087 | C | TYR | A | 461 | 18.567 | 34.737 | 89.828 | 1.00 | 31.03 | C |
| ATOM | 1088 | O | TYR | A | 461 | 18.869 | 33.573 | 90.097 | 1.00 | 30.81 | O |
| ATOM | 1089 | CB | TYR | A | 461 | 17.528 | 35.929 | 91.766 | 1.00 | 31.44 | C |
| ATOM | 1090 | CG | TYR | A | 461 | 16.314 | 36.501 | 92.475 | 1.00 | 31.69 | C |
| ATOM | 1091 | CD1 | TYR | A | 461 | 15.691 | 37.661 | 92.014 | 1.00 | 31.59 | C |
| ATOM | 1092 | CD2 | TYR | A | 461 | 15.807 | 35.896 | 93.629 | 1.00 | 32.41 | C |
| ATOM | 1093 | CE1 | TYR | A | 461 | 14.585 | 38.195 | 92.667 | 1.00 | 31.82 | C |
| ATOM | 1094 | CE2 | TYR | A | 461 | 14.692 | 36.423 | 94.298 | 1.00 | 32.05 | C |
| ATOM | 1095 | CZ | TYR | A | 461 | 14.089 | 37.574 | 93.807 | 1.00 | 32.14 | C |
| ATOM | 1096 | OH | TYR | A | 461 | 12.992 | 38.112 | 94.447 | 1.00 | 31.78 | O |
| ATOM | 1097 | N | GLN | A | 462 | 19.323 | 35.520 | 89.065 | 1.00 | 30.90 | N |
| ATOM | 1098 | CA | GLN | A | 462 | 20.641 | 35.086 | 88.610 | 1.00 | 30.97 | C |
| ATOM | 1099 | C | GLN | A | 462 | 21.596 | 34.949 | 89.800 | 1.00 | 31.28 | C |
| ATOM | 1100 | O | GLN | A | 462 | 21.537 | 35.733 | 90.752 | 1.00 | 31.18 | O |
| ATOM | 1101 | CB | GLN | A | 462 | 21.218 | 36.031 | 87.544 | 1.00 | 30.83 | C |
| ATOM | 1102 | CG | GLN | A | 462 | 22.506 | 35.512 | 86.898 | 1.00 | 30.35 | C |
| ATOM | 1103 | CD | GLN | A | 462 | 22.924 | 36.274 | 85.654 | 1.00 | 30.59 | C |
| ATOM | 1104 | OE1 | GLN | A | 462 | 22.653 | 37.468 | 85.505 | 1.00 | 30.10 | O |
| ATOM | 1105 | NE2 | GLN | A | 462 | 23.605 | 35.583 | 84.756 | 1.00 | 30.48 | N |
| ATOM | 1106 | N | ILE | A | 463 | 22.455 | 33.936 | 89.732 | 1.00 | 31.51 | N |
| ATOM | 1107 | CA | ILE | A | 463 | 23.425 | 33.639 | 90.776 | 1.00 | 31.87 | C |
| ATOM | 1108 | C | ILE | A | 463 | 24.729 | 34.424 | 90.572 | 1.00 | 32.48 | C |
| ATOM | 1109 | O | ILE | A | 463 | 25.079 | 34.784 | 89.442 | 1.00 | 32.35 | O |
| ATOM | 1110 | CB | ILE | A | 463 | 23.691 | 32.107 | 90.851 | 1.00 | 31.66 | C |
| ATOM | 1111 | CG1 | ILE | A | 463 | 22.452 | 31.392 | 91.404 | 1.00 | 31.56 | C |
| ATOM | 1112 | CG2 | ILE | A | 463 | 24.926 | 31.790 | 91.703 | 1.00 | 31.59 | C |
| ATOM | 1113 | CD1 | ILE | A | 463 | 22.508 | 29.881 | 91.330 | 1.00 | 31.58 | C |
| ATOM | 1114 | N | LEU | A | 464 | 25.418 | 34.707 | 91.679 | 1.00 | 33.20 | N |
| ATOM | 1115 | CA | LEU | A | 464 | 26.769 | 35.267 | 91.651 | 1.00 | 33.99 | C |
| ATOM | 1116 | C | LEU | A | 464 | 27.719 | 34.414 | 92.488 | 1.00 | 34.80 | C |
| ATOM | 1117 | O | LEU | A | 464 | 27.342 | 33.913 | 93.546 | 1.00 | 35.10 | O |
| ATOM | 1118 | CB | LEU | A | 464 | 26.777 | 36.715 | 92.156 | 1.00 | 33.79 | C |
| ATOM | 1119 | CG | LEU | A | 464 | 26.077 | 37.800 | 91.325 | 1.00 | 33.47 | C |
| ATOM | 1120 | CD1 | LEU | A | 464 | 25.934 | 39.075 | 92.145 | 1.00 | 33.53 | C |
| ATOM | 1121 | CD2 | LEU | A | 464 | 26.799 | 38.088 | 90.011 | 1.00 | 32.46 | C |
| ATOM | 1122 | N | TYR | A | 465 | 28.950 | 34.244 | 92.019 | 1.00 | 35.71 | N |
| ATOM | 1123 | CA | TYR | A | 465 | 29.946 | 33.497 | 92.786 | 1.00 | 36.81 | C |
| ATOM | 1124 | C | TYR | A | 465 | 31.054 | 34.400 | 93.309 | 1.00 | 37.67 | C |
| ATOM | 1125 | O | TYR | A | 465 | 31.750 | 35.059 | 92.529 | 1.00 | 37.90 | O |
| ATOM | 1126 | CB | TYR | A | 465 | 30.519 | 32.348 | 91.958 | 1.00 | 36.45 | C |
| ATOM | 1127 | CG | TYR | A | 465 | 29.486 | 31.298 | 91.633 | 1.00 | 36.63 | C |
| ATOM | 1128 | CD1 | TYR | A | 465 | 29.124 | 30.335 | 92.577 | 1.00 | 36.31 | C |
| ATOM | 1129 | CD2 | TYR | A | 465 | 28.854 | 31.274 | 90.389 | 1.00 | 35.95 | C |
| ATOM | 1130 | CE1 | TYR | A | 465 | 28.171 | 29.373 | 92.289 | 1.00 | 35.78 | C |
| ATOM | 1131 | CE2 | TYR | A | 465 | 27.901 | 30.312 | 90.092 | 1.00 | 35.68 | C |
| ATOM | 1132 | CZ | TYR | A | 465 | 27.562 | 29.367 | 91.047 | 1.00 | 35.72 | C |
| ATOM | 1133 | OH | TYR | A | 465 | 26.617 | 28.410 | 90.763 | 1.00 | 35.88 | O |
| ATOM | 1134 | N | ARG | A | 466 | 31.193 | 34.440 | 94.634 | 1.00 | 38.77 | N |
| ATOM | 1135 | CA | ARG | A | 466 | 32.236 | 35.234 | 95.283 | 1.00 | 39.86 | C |
| ATOM | 1136 | C | ARG | A | 466 | 33.581 | 34.514 | 95.260 | 1.00 | 40.53 | C |
| ATOM | 1137 | O | ARG | A | 466 | 33.645 | 33.288 | 95.360 | 1.00 | 40.57 | O |
| ATOM | 1138 | CB | ARG | A | 466 | 31.857 | 35.597 | 96.723 | 1.00 | 39.72 | C |
| ATOM | 1139 | CG | ARG | A | 466 | 32.639 | 36.797 | 97.254 | 1.00 | 40.77 | C |
| ATOM | 1140 | CD | ARG | A | 466 | 32.776 | 36.805 | 98.767 | 1.00 | 41.70 | C |
| ATOM | 1141 | NE | ARG | A | 466 | 31.675 | 37.514 | 99.415 | 1.00 | 42.18 | N |
| ATOM | 1142 | CZ | ARG | A | 466 | 31.712 | 38.791 | 99.786 | 1.00 | 41.94 | C |
| ATOM | 1143 | NH1 | ARG | A | 466 | 32.801 | 39.520 | 99.575 | 1.00 | 42.15 | N |
| ATOM | 1144 | NH2 | ARG | A | 466 | 30.653 | 39.340 | 100.367 | 1.00 | 41.42 | N |
| ATOM | 1145 | N | GLU | A | 467 | 34.650 | 35.296 | 95.125 | 1.00 | 41.52 | N |
| ATOM | 1146 | CA | GLU | A | 467 | 36.018 | 34.783 | 95.150 | 1.00 | 42.71 | C |
| ATOM | 1147 | C | GLU | A | 467 | 36.284 | 33.988 | 96.427 | 1.00 | 42.58 | C |
| ATOM | 1148 | O | GLU | A | 467 | 35.961 | 34.439 | 97.529 | 1.00 | 42.43 | O |
| ATOM | 1149 | CB | GLU | A | 467 | 37.026 | 35.934 | 95.013 | 1.00 | 42.52 | C |
| ATOM | 1150 | CG | GLU | A | 467 | 36.793 | 36.815 | 93.779 | 1.00 | 43.91 | C |
| ATOM | 1151 | CD | GLU | A | 467 | 37.848 | 37.911 | 93.586 | 1.00 | 44.52 | C |
| ATOM | 1152 | OE1 | GLU | A | 467 | 38.401 | 38.426 | 94.596 | 1.00 | 45.70 | O |
| ATOM | 1153 | OE2 | GLU | A | 467 | 38.107 | 38.264 | 92.406 | 1.00 | 46.43 | O |
| ATOM | 1154 | N | GLY | A | 468 | 36.839 | 32.791 | 96.260 | 1.00 | 42.99 | N |
| ATOM | 1155 | CA | GLY | A | 468 | 37.223 | 31.935 | 97.382 | 1.00 | 43.41 | C |
| ATOM | 1156 | C | GLY | A | 468 | 36.083 | 31.287 | 98.148 | 1.00 | 43.72 | C |
| ATOM | 1157 | O | GLY | A | 468 | 36.264 | 30.225 | 98.740 | 1.00 | 43.91 | O |
| ATOM | 1158 | N | GLU | A | 469 | 34.913 | 31.924 | 98.131 | 1.00 | 43.84 | N |
| ATOM | 1159 | CA | GLU | A | 469 | 33.741 | 31.476 | 98.891 | 1.00 | 43.93 | C |
| ATOM | 1160 | C | GLU | A | 469 | 32.773 | 30.613 | 98.064 | 1.00 | 43.78 | C |
| ATOM | 1161 | O | GLU | A | 469 | 32.518 | 30.890 | 96.888 | 1.00 | 43.77 | O |
| ATOM | 1162 | CB | GLU | A | 469 | 33.012 | 32.701 | 99.446 | 1.00 | 43.93 | C |
| ATOM | 1163 | CG | GLU | A | 469 | 31.947 | 32.426 | 100.496 | 1.00 | 44.20 | C |
| ATOM | 1164 | CD | GLU | A | 469 | 31.289 | 33.709 | 100.977 | 1.00 | 44.52 | C |
| ATOM | 1165 | OE1 | GLU | A | 469 | 32.020 | 34.704 | 101.199 | 1.00 | 45.61 | O |
| ATOM | 1166 | OE2 | GLU | A | 469 | 30.048 | 33.728 | 101.131 | 1.00 | 44.33 | O |
| ATOM | 1167 | N | LYS | A | 470 | 32.242 | 29.568 | 98.692 | 1.00 | 43.64 | N |
| ATOM | 1168 | CA | LYS | A | 470 | 31.237 | 28.706 | 98.076 | 1.00 | 43.38 | C |
| ATOM | 1169 | C | LYS | A | 470 | 29.845 | 29.144 | 98.527 | 1.00 | 43.57 | C |
| ATOM | 1170 | O | LYS | A | 470 | 29.628 | 29.407 | 99.716 | 1.00 | 43.58 | O |
| ATOM | 1171 | CB | LYS | A | 470 | 31.478 | 27.244 | 98.466 | 1.00 | 43.10 | C |
| ATOM | 1172 | CG | LYS | A | 470 | 30.372 | 26.284 | 98.048 | 1.00 | 42.93 | C |
| ATOM | 1173 | CD | LYS | A | 470 | 30.552 | 24.908 | 98.672 | 1.00 | 43.10 | C |
| ATOM | 1174 | CE | LYS | A | 470 | 29.463 | 23.940 | 98.218 | 1.00 | 42.35 | C |
| ATOM | 1175 | NZ | LYS | A | 470 | 29.695 | 22.557 | 98.730 | 1.00 | 41.88 | N |
| ATOM | 1176 | N | GLY | A | 471 | 28.907 | 29.226 | 97.584 | 1.00 | 43.45 | N |
| ATOM | 1177 | CA | GLY | A | 471 | 27.521 | 29.509 | 97.938 | 1.00 | 43.50 | C |
| ATOM | 1178 | C | GLY | A | 471 | 26.746 | 30.403 | 96.992 | 1.00 | 43.60 | C |
| ATOM | 1179 | O | GLY | A | 471 | 27.325 | 31.072 | 96.132 | 1.00 | 43.40 | O |
| ATOM | 1180 | N | ALA | A | 472 | 25.426 | 30.403 | 97.189 | 1.00 | 43.70 | N |
| ATOM | 1181 | CA | ALA | A | 472 | 24.447 | 31.134 | 96.374 | 1.00 | 43.79 | C |
| ATOM | 1182 | C | ALA | A | 472 | 24.861 | 32.539 | 95.941 | 1.00 | 43.90 | C |
| ATOM | 1183 | O | ALA | A | 472 | 25.612 | 32.680 | 94.984 | 1.00 | 44.39 | O |
| ATOM | 1184 | CB | ALA | A | 472 | 23.082 | 31.167 | 97.086 | 1.00 | 43.83 | C |
| ATOM | 1185 | N | TYR | A | 473 | 24.382 | 33.564 | 96.646 | 1.00 | 43.76 | N |
| ATOM | 1186 | CA | TYR | A | 473 | 24.408 | 34.964 | 96.173 | 1.00 | 43.85 | C |
| ATOM | 1187 | C | TYR | A | 473 | 23.495 | 35.144 | 94.959 | 1.00 | 43.83 | C |
| ATOM | 1188 | O | TYR | A | 473 | 23.697 | 34.517 | 93.917 | 1.00 | 43.65 | O |
| ATOM | 1189 | CB | TYR | A | 473 | 25.827 | 35.465 | 95.844 | 1.00 | 43.88 | C |
| ATOM | 1190 | CG | TYR | A | 473 | 26.710 | 35.768 | 97.038 | 1.00 | 43.94 | C |
| ATOM | 1191 | CD1 | TYR | A | 473 | 26.581 | 36.965 | 97.744 | 1.00 | 43.71 | C |
| ATOM | 1192 | CD2 | TYR | A | 473 | 27.693 | 34.866 | 97.443 | 1.00 | 43.92 | C |
| ATOM | 1193 | CE1 | TYR | A | 473 | 27.397 | 37.245 | 98.838 | 1.00 | 43.70 | C |
| ATOM | 1194 | CE2 | TYR | A | 473 | 28.512 | 35.135 | 98.528 | 1.00 | 44.09 | C |
| ATOM | 1195 | CZ | TYR | A | 473 | 28.360 | 36.324 | 99.223 | 1.00 | 44.20 | C |
| ATOM | 1196 | OH | TYR | A | 473 | 29.178 | 36.582 | 100.301 | 1.00 | 43.91 | O |
| ATOM | 1197 | N | THR | A | 474 | 22.490 | 36.003 | 95.099 | 1.00 | 43.97 | N |
| ATOM | 1198 | CA | THR | A | 474 | 21.526 | 36.237 | 94.023 | 1.00 | 44.30 | C |
| ATOM | 1199 | C | THR | A | 474 | 21.522 | 37.696 | 93.569 | 1.00 | 44.53 | C |
| ATOM | 1200 | O | THR | A | 474 | 22.269 | 38.503 | 94.108 | 1.00 | 44.67 | O |
| ATOM | 1201 | CB | THR | A | 474 | 20.108 | 35.793 | 94.429 | 1.00 | 44.25 | C |
| ATOM | 1202 | OG1 | THR | A | 474 | 19.760 | 36.379 | 95.690 | 1.00 | 44.06 | O |
| ATOM | 1203 | CG2 | THR | A | 474 | 20.038 | 34.274 | 94.524 | 1.00 | 43.92 | C |
| ATOM | 1204 | N | ILE | A | 475 | 20.714 | 38.021 | 92.558 | 1.00 | 44.83 | N |
| ATOM | 1205 | CA | ILE | A | 475 | 20.557 | 39.406 | 92.090 | 1.00 | 45.16 | C |
| ATOM | 1206 | C | ILE | A | 475 | 19.078 | 39.760 | 92.080 | 1.00 | 45.58 | C |
| ATOM | 1207 | O | ILE | A | 475 | 18.317 | 39.248 | 91.258 | 1.00 | 45.86 | O |
| ATOM | 1208 | CB | ILE | A | 475 | 21.143 | 39.634 | 90.673 | 1.00 | 45.04 | C |
| ATOM | 1209 | CG1 | ILE | A | 475 | 22.626 | 39.254 | 90.627 | 1.00 | 44.87 | C |
| ATOM | 1210 | CG2 | ILE | A | 475 | 20.947 | 41.092 | 90.243 | 1.00 | 45.27 | C |
| ATOM | 1211 | CD1 | ILE | A | 475 | 23.206 | 39.163 | 89.230 | 1.00 | 44.94 | C |
| ATOM | 1212 | N | ARG | A | 476 | 18.676 | 40.642 | 92.989 | 1.00 | 46.07 | N |
| ATOM | 1213 | CA | ARG | A | 476 | 17.262 | 40.925 | 93.205 | 1.00 | 46.46 | C |
| ATOM | 1214 | C | ARG | A | 476 | 16.931 | 42.393 | 92.960 | 1.00 | 47.15 | C |
| ATOM | 1215 | O | ARG | A | 476 | 17.814 | 43.190 | 92.646 | 1.00 | 47.38 | O |
| ATOM | 1216 | CB | ARG | A | 476 | 16.855 | 40.523 | 94.630 | 1.00 | 46.53 | C |
| ATOM | 1217 | CG | ARG | A | 476 | 17.318 | 39.130 | 95.073 | 1.00 | 46.02 | C |
| ATOM | 1218 | CD | ARG | A | 476 | 16.674 | 38.721 | 96.393 | 1.00 | 45.61 | C |
| ATOM | 1219 | NE | ARG | A | 476 | 17.381 | 37.617 | 97.048 | 1.00 | 43.22 | N |
| ATOM | 1220 | CZ | ARG | A | 476 | 16.890 | 36.897 | 98.055 | 1.00 | 41.95 | C |
| ATOM | 1221 | NH1 | ARG | A | 476 | 15.677 | 37.147 | 98.532 | 1.00 | 41.15 | N |
| ATOM | 1222 | NH2 | ARG | A | 476 | 17.612 | 35.921 | 98.586 | 1.00 | 41.08 | N |
| ATOM | 1223 | N | GLU | A | 477 | 15.644 | 42.718 | 93.070 | 1.00 | 47.83 | N |
| ATOM | 1224 | CA | GLU | A | 477 | 15.145 | 44.097 | 93.210 | 1.00 | 48.66 | C |
| ATOM | 1225 | C | GLU | A | 477 | 15.764 | 45.147 | 92.277 | 1.00 | 48.96 | C |
| ATOM | 1226 | O | GLU | A | 477 | 15.403 | 45.216 | 91.099 | 1.00 | 49.26 | O |
| ATOM | 1227 | CB | GLU | A | 477 | 15.246 | 44.540 | 94.673 | 1.00 | 48.88 | C |
| ATOM | 1228 | CG | GLU | A | 477 | 14.525 | 43.606 | 95.644 | 1.00 | 49.75 | C |
| ATOM | 1229 | CD | GLU | A | 477 | 15.094 | 43.654 | 97.047 | 1.00 | 50.82 | C |
| ATOM | 1230 | OE1 | GLU | A | 477 | 16.171 | 44.264 | 97.244 | 1.00 | 51.19 | O |
| ATOM | 1231 | OE2 | GLU | A | 477 | 14.462 | 43.073 | 97.956 | 1.00 | 51.58 | O |
| ATOM | 1232 | N | ASN | A | 478 | 16.670 | 45.970 | 92.803 | 1.00 | 49.16 | N |
| ATOM | 1233 | CA | ASN | A | 478 | 17.307 | 47.019 | 92.002 | 1.00 | 49.60 | C |
| ATOM | 1234 | C | ASN | A | 478 | 18.776 | 46.694 | 91.739 | 1.00 | 49.57 | C |
| ATOM | 1235 | O | ASN | A | 478 | 19.636 | 47.579 | 91.715 | 1.00 | 49.52 | O |
| ATOM | 1236 | CB | ASN | A | 478 | 17.162 | 48.393 | 92.677 | 1.00 | 49.75 | C |
| ATOM | 1237 | CG | ASN | A | 478 | 17.332 | 49.562 | 91.694 | 1.00 | 50.95 | C |
| ATOM | 1238 | OD1 | ASN | A | 478 | 16.850 | 49.520 | 90.554 | 1.00 | 51.93 | O |
| ATOM | 1239 | ND2 | ASN | A | 478 | 18.013 | 50.617 | 92.143 | 1.00 | 51.48 | N |
| ATOM | 1240 | N | GLY | A | 479 | 19.052 | 45.409 | 91.544 | 1.00 | 49.58 | N |
| ATOM | 1241 | CA | GLY | A | 479 | 20.420 | 44.932 | 91.399 | 1.00 | 49.48 | C |
| ATOM | 1242 | C | GLY | A | 479 | 21.101 | 44.677 | 92.727 | 1.00 | 49.41 | C |
| ATOM | 1243 | O | GLY | A | 479 | 22.273 | 44.296 | 92.760 | 1.00 | 49.20 | O |
| ATOM | 1244 | N | THR | A | 480 | 20.366 | 44.897 | 93.820 | 1.00 | 49.52 | N |
| ATOM | 1245 | CA | THR | A | 480 | 20.839 | 44.590 | 95.171 | 1.00 | 49.47 | C |
| ATOM | 1246 | C | THR | A | 480 | 21.220 | 43.116 | 95.210 | 1.00 | 49.49 | C |
| ATOM | 1247 | O | THR | A | 480 | 20.402 | 42.254 | 94.879 | 1.00 | 49.41 | O |
| ATOM | 1248 | CB | THR | A | 480 | 19.749 | 44.867 | 96.233 | 1.00 | 49.46 | C |
| ATOM | 1249 | OG1 | THR | A | 480 | 19.269 | 46.209 | 96.096 | 1.00 | 49.58 | O |
| ATOM | 1250 | CG2 | THR | A | 480 | 20.299 | 44.678 | 97.636 | 1.00 | 49.27 | C |
| ATOM | 1251 | N | VAL | A | 481 | 22.463 | 42.837 | 95.600 | 1.00 | 49.60 | N |
| ATOM | 1252 | CA | VAL | A | 481 | 23.004 | 41.477 | 95.530 | 1.00 | 49.75 | C |
| ATOM | 1253 | C | VAL | A | 481 | 22.244 | 40.482 | 96.422 | 1.00 | 50.03 | C |
| ATOM | 1254 | O | VAL | A | 481 | 21.356 | 39.791 | 95.923 | 1.00 | 50.33 | O |
| ATOM | 1255 | CB | VAL | A | 481 | 24.545 | 41.437 | 95.725 | 1.00 | 49.65 | C |
| ATOM | 1256 | CG1 | VAL | A | 481 | 25.062 | 40.001 | 95.770 | 1.00 | 49.63 | C |
| ATOM | 1257 | CG2 | VAL | A | 481 | 25.227 | 42.189 | 94.591 | 1.00 | 49.47 | C |
| ATOM | 1258 | N | TYR | A | 482 | 22.559 | 40.430 | 97.718 | 1.00 | 50.05 | N |
| ATOM | 1259 | CA | TYR | A | 482 | 21.996 | 39.426 | 98.644 | 1.00 | 50.04 | C |
| ATOM | 1260 | C | TYR | A | 482 | 22.929 | 38.237 | 98.854 | 1.00 | 50.23 | C |
| ATOM | 1261 | O | TYR | A | 482 | 23.433 | 37.650 | 97.894 | 1.00 | 50.16 | O |
| ATOM | 1262 | CB | TYR | A | 482 | 20.610 | 38.922 | 98.204 | 1.00 | 49.83 | C |
| ATOM | 1263 | CG | TYR | A | 482 | 19.457 | 39.809 | 98.616 | 1.00 | 49.68 | C |
| ATOM | 1264 | CD1 | TYR | A | 482 | 18.632 | 39.454 | 99.682 | 1.00 | 49.36 | C |
| ATOM | 1265 | CD2 | TYR | A | 482 | 19.190 | 41.004 | 97.942 | 1.00 | 49.42 | C |
| ATOM | 1266 | CE1 | TYR | A | 482 | 17.568 | 40.263 | 100.067 | 1.00 | 49.38 | C |
| ATOM | 1267 | CE2 | TYR | A | 482 | 18.132 | 41.820 | 98.319 | 1.00 | 49.59 | C |
| ATOM | 1268 | CZ | TYR | A | 482 | 17.324 | 41.443 | 99.382 | 1.00 | 49.70 | C |
| ATOM | 1269 | OH | TYR | A | 482 | 16.274 | 42.249 | 99.763 | 1.00 | 49.75 | O |
| ATOM | 1270 | N | THR | A | 483 | 23.136 | 37.895 | 100.124 | 1.00 | 50.47 | N |
| ATOM | 1271 | CA | THR | A | 483 | 23.974 | 36.772 | 100.545 | 1.00 | 50.90 | C |
| ATOM | 1272 | C | THR | A | 483 | 23.199 | 35.444 | 100.427 | 1.00 | 51.05 | C |
| ATOM | 1273 | O | THR | A | 483 | 21.969 | 35.465 | 100.341 | 1.00 | 51.11 | O |
| ATOM | 1274 | CB | THR | A | 483 | 24.499 | 37.012 | 102.002 | 1.00 | 50.89 | C |
| ATOM | 1275 | OG1 | THR | A | 483 | 25.339 | 35.932 | 102.421 | 1.00 | 51.63 | O |
| ATOM | 1276 | CG2 | THR | A | 483 | 23.361 | 37.151 | 102.981 | 1.00 | 50.61 | C |
| ATOM | 1277 | N | PRO | A | 484 | 23.908 | 34.290 | 100.388 | 1.00 | 51.21 | N |
| ATOM | 1278 | CA | PRO | A | 484 | 23.226 | 32.993 | 100.495 | 1.00 | 51.51 | C |
| ATOM | 1279 | C | PRO | A | 484 | 22.342 | 32.841 | 101.743 | 1.00 | 52.04 | C |
| ATOM | 1280 | O | PRO | A | 484 | 21.564 | 31.892 | 101.820 | 1.00 | 52.25 | O |
| ATOM | 1281 | CB | PRO | A | 484 | 24.382 | 31.991 | 100.557 | 1.00 | 51.32 | C |
| ATOM | 1282 | CG | PRO | A | 484 | 25.497 | 32.661 | 99.879 | 1.00 | 51.09 | C |
| ATOM | 1283 | CD | PRO | A | 484 | 25.360 | 34.119 | 100.191 | 1.00 | 51.11 | C |
| ATOM | 1284 | N | ASP | A | 485 | 22.468 | 33.760 | 102.702 | 1.00 | 52.65 | N |
| ATOM | 1285 | CA | ASP | A | 485 | 21.617 | 33.788 | 103.898 | 1.00 | 53.28 | C |
| ATOM | 1286 | C | ASP | A | 485 | 20.230 | 34.359 | 103.611 | 1.00 | 53.70 | C |
| ATOM | 1287 | O | ASP | A | 485 | 19.280 | 34.110 | 104.357 | 1.00 | 53.74 | O |
| ATOM | 1288 | CB | ASP | A | 485 | 22.265 | 34.629 | 105.008 | 1.00 | 53.34 | C |
| ATOM | 1289 | CG | ASP | A | 485 | 23.160 | 33.818 | 105.930 | 1.00 | 53.68 | C |
| ATOM | 1290 | OD1 | ASP | A | 485 | 23.146 | 32.570 | 105.859 | 1.00 | 54.20 | O |
| ATOM | 1291 | OD2 | ASP | A | 485 | 23.876 | 34.440 | 106.745 | 1.00 | 54.15 | O |
| ATOM | 1292 | N | GLY | A | 486 | 20.123 | 35.140 | 102.540 | 1.00 | 54.20 | N |
| ATOM | 1293 | CA | GLY | A | 486 | 18.902 | 35.880 | 102.244 | 1.00 | 54.87 | C |
| ATOM | 1294 | C | GLY | A | 486 | 18.923 | 37.262 | 102.871 | 1.00 | 55.36 | C |
| ATOM | 1295 | O | GLY | A | 486 | 17.933 | 37.992 | 102.815 | 1.00 | 55.25 | O |
| ATOM | 1296 | N | LYS | A | 487 | 20.056 | 37.618 | 103.474 | 1.00 | 56.00 | N |
| ATOM | 1297 | CA | LYS | A | 487 | 20.240 | 38.938 | 104.072 | 1.00 | 56.75 | C |
| ATOM | 1298 | C | LYS | A | 487 | 20.544 | 39.941 | 102.978 | 1.00 | 57.05 | C |
| ATOM | 1299 | O | LYS | A | 487 | 21.427 | 39.720 | 102.147 | 1.00 | 56.96 | O |
| ATOM | 1300 | CB | LYS | A | 487 | 21.367 | 38.935 | 105.115 | 1.00 | 56.57 | C |
| ATOM | 1301 | CG | LYS | A | 487 | 21.052 | 38.146 | 106.380 | 1.00 | 57.10 | C |
| ATOM | 1302 | CD | LYS | A | 487 | 22.250 | 38.073 | 107.318 | 1.00 | 57.24 | C |
| ATOM | 1303 | CE | LYS | A | 487 | 21.909 | 37.278 | 108.574 | 1.00 | 58.10 | C |
| ATOM | 1304 | NZ | LYS | A | 487 | 23.053 | 37.209 | 109.523 | 1.00 | 58.35 | N |
| ATOM | 1305 | N | ALA | A | 488 | 19.795 | 41.038 | 102.976 | 1.00 | 57.66 | N |
| ATOM | 1306 | CA | ALA | A | 488 | 20.016 | 42.116 | 102.024 | 1.00 | 58.21 | C |
| ATOM | 1307 | C | ALA | A | 488 | 21.345 | 42.791 | 102.321 | 1.00 | 58.52 | C |
| ATOM | 1308 | O | ALA | A | 488 | 21.388 | 43.868 | 102.919 | 1.00 | 58.69 | O |
| ATOM | 1309 | CB | ALA | A | 488 | 18.871 | 43.126 | 102.080 | 1.00 | 58.23 | C |
| ATOM | 1310 | N | THR | A | 489 | 22.434 | 42.134 | 101.927 | 1.00 | 58.91 | N |
| ATOM | 1311 | CA | THR | A | 489 | 23.758 | 42.720 | 102.064 | 1.00 | 59.21 | C |
| ATOM | 1312 | C | THR | A | 489 | 23.867 | 43.829 | 101.039 | 1.00 | 59.31 | C |
| ATOM | 1313 | O | THR | A | 489 | 24.367 | 43.625 | 99.926 | 1.00 | 59.47 | O |
| ATOM | 1314 | CB | THR | A | 489 | 24.897 | 41.697 | 101.867 | 1.00 | 59.35 | C |
| ATOM | 1315 | OG1 | THR | A | 489 | 24.732 | 41.024 | 100.610 | 1.00 | 59.36 | O |
| ATOM | 1316 | CG2 | THR | A | 489 | 24.925 | 40.684 | 103.016 | 1.00 | 59.44 | C |
| ATOM | 1317 | N | ASP | A | 490 | 23.354 | 44.995 | 101.428 | 1.00 | 59.49 | N |
| ATOM | 1318 | CA | ASP | A | 490 | 23.426 | 46.208 | 100.627 | 1.00 | 59.47 | C |
| ATOM | 1319 | C | ASP | A | 490 | 24.878 | 46.589 | 100.392 | 1.00 | 59.15 | C |
| ATOM | 1320 | O | ASP | A | 490 | 25.767 | 45.732 | 100.415 | 1.00 | 59.07 | O |
| ATOM | 1321 | CB | ASP | A | 490 | 22.670 | 47.362 | 101.306 | 1.00 | 59.79 | C |
| ATOM | 1322 | CG | ASP | A | 490 | 21.195 | 47.413 | 100.923 | 1.00 | 60.56 | C |
| ATOM | 1323 | OD1 | ASP | A | 490 | 20.836 | 46.996 | 99.797 | 1.00 | 61.32 | O |
| ATOM | 1324 | OD2 | ASP | A | 490 | 20.392 | 47.889 | 101.754 | 1.00 | 61.61 | O |
| ATOM | 1325 | N | TYR | A | 491 | 25.121 | 47.881 | 100.192 | 1.00 | 58.80 | N |
| ATOM | 1326 | CA | TYR | A | 491 | 26.371 | 48.340 | 99.612 | 1.00 | 58.40 | C |
| ATOM | 1327 | C | TYR | A | 491 | 26.392 | 47.820 | 98.160 | 1.00 | 57.75 | C |
| ATOM | 1328 | O | TYR | A | 491 | 26.462 | 48.607 | 97.216 | 1.00 | 57.86 | O |
| ATOM | 1329 | CB | TYR | A | 491 | 27.596 | 47.847 | 100.417 | 1.00 | 58.72 | C |
| ATOM | 1330 | CG | TYR | A | 491 | 27.702 | 48.334 | 101.862 | 1.00 | 59.19 | C |
| ATOM | 1331 | CD1 | TYR | A | 491 | 28.821 | 49.054 | 102.294 | 1.00 | 59.58 | C |
| ATOM | 1332 | CD2 | TYR | A | 491 | 26.701 | 48.050 | 102.804 | 1.00 | 59.90 | C |
| ATOM | 1333 | CE1 | TYR | A | 491 | 28.935 | 49.494 | 103.624 | 1.00 | 59.77 | C |
| ATOM | 1334 | CE2 | TYR | A | 491 | 26.801 | 48.486 | 104.133 | 1.00 | 59.88 | C |
| ATOM | 1335 | CZ | TYR | A | 491 | 27.920 | 49.208 | 104.537 | 1.00 | 60.00 | C |
| ATOM | 1336 | OH | TYR | A | 491 | 28.021 | 49.636 | 105.848 | 1.00 | 59.47 | O |
| ATOM | 1337 | N | ARG | A | 492 | 26.268 | 46.500 | 98.004 | 1.00 | 56.87 | N |
| ATOM | 1338 | CA | ARG | A | 492 | 26.362 | 45.797 | 96.714 | 1.00 | 55.99 | C |
| ATOM | 1339 | C | ARG | A | 492 | 25.170 | 46.005 | 95.772 | 1.00 | 55.30 | C |
| ATOM | 1340 | O | ARG | A | 492 | 24.038 | 45.624 | 96.097 | 1.00 | 55.16 | O |
| ATOM | 1341 | CB | ARG | A | 492 | 26.530 | 44.291 | 96.955 | 1.00 | 56.02 | C |
| ATOM | 1342 | CG | ARG | A | 492 | 27.615 | 43.899 | 97.936 | 1.00 | 55.82 | C |
| ATOM | 1343 | CD | ARG | A | 492 | 28.978 | 44.160 | 97.347 | 1.00 | 55.09 | C |
| ATOM | 1344 | NE | ARG | A | 492 | 30.038 | 43.774 | 98.264 | 1.00 | 54.67 | N |
| ATOM | 1345 | CZ | ARG | A | 492 | 31.307 | 44.130 | 98.118 | 1.00 | 54.88 | C |
| ATOM | 1346 | NH1 | ARG | A | 492 | 31.667 | 44.886 | 97.088 | 1.00 | 55.12 | N |
| ATOM | 1347 | NH2 | ARG | A | 492 | 32.214 | 43.736 | 99.003 | 1.00 | 54.34 | N |
| ATOM | 1348 | N | VAL | A | 493 | 25.436 | 46.602 | 94.609 | 1.00 | 54.40 | N |
| ATOM | 1349 | CA | VAL | A | 493 | 24.464 | 46.652 | 93.505 | 1.00 | 53.50 | C |
| ATOM | 1350 | C | VAL | A | 493 | 25.100 | 46.302 | 92.158 | 1.00 | 52.84 | C |
| ATOM | 1351 | O | VAL | A | 493 | 26.219 | 46.722 | 91.853 | 1.00 | 52.57 | O |
| ATOM | 1352 | CB | VAL | A | 493 | 23.677 | 48.008 | 93.393 | 1.00 | 53.56 | C |
| ATOM | 1353 | CG1 | VAL | A | 493 | 22.510 | 48.056 | 94.378 | 1.00 | 53.58 | C |
| ATOM | 1354 | CG2 | VAL | A | 493 | 24.593 | 49.226 | 93.550 | 1.00 | 53.62 | C |
| ATOM | 1355 | N | VAL | A | 494 | 24.376 | 45.504 | 91.379 | 1.00 | 52.22 | N |
| ATOM | 1356 | CA | VAL | A | 494 | 24.689 | 45.246 | 89.979 | 1.00 | 51.43 | C |
| ATOM | 1357 | C | VAL | A | 494 | 23.725 | 46.093 | 89.169 | 1.00 | 51.11 | C |
| ATOM | 1358 | O | VAL | A | 494 | 22.545 | 46.183 | 89.508 | 1.00 | 50.94 | O |
| ATOM | 1359 | CB | VAL | A | 494 | 24.469 | 43.757 | 89.609 | 1.00 | 51.44 | C |
| ATOM | 1360 | CG1 | VAL | A | 494 | 24.589 | 43.542 | 88.100 | 1.00 | 51.30 | C |
| ATOM | 1361 | CG2 | VAL | A | 494 | 25.442 | 42.859 | 90.356 | 1.00 | 50.97 | C |
| ATOM | 1362 | N | VAL | A | 495 | 24.210 | 46.724 | 88.109 | 1.00 | 50.70 | N |
| ATOM | 1363 | CA | VAL | A | 495 | 23.318 | 47.522 | 87.280 | 1.00 | 50.49 | C |
| ATOM | 1364 | C | VAL | A | 495 | 22.611 | 46.635 | 86.256 | 1.00 | 50.33 | C |
| ATOM | 1365 | O | VAL | A | 495 | 23.245 | 45.812 | 85.588 | 1.00 | 50.33 | O |
| ATOM | 1366 | CB | VAL | A | 495 | 24.035 | 48.701 | 86.569 | 1.00 | 50.59 | C |
| ATOM | 1367 | CG1 | VAL | A | 495 | 23.036 | 49.816 | 86.276 | 1.00 | 50.69 | C |
| ATOM | 1368 | CG2 | VAL | A | 495 | 25.177 | 49.253 | 87.414 | 1.00 | 50.31 | C |
| ATOM | 1369 | N | ASP | A | 496 | 21.292 | 46.789 | 86.166 | 1.00 | 50.04 | N |
| ATOM | 1370 | CA | ASP | A | 496 | 20.503 | 46.186 | 85.085 | 1.00 | 49.87 | C |
| ATOM | 1371 | C | ASP | A | 496 | 19.498 | 47.131 | 84.418 | 1.00 | 49.79 | C |
| ATOM | 1372 | O | ASP | A | 496 | 19.067 | 46.859 | 83.298 | 1.00 | 50.24 | O |
| ATOM | 1373 | CB | ASP | A | 496 | 19.841 | 44.863 | 85.497 | 1.00 | 49.91 | C |
| ATOM | 1374 | CG | ASP | A | 496 | 19.632 | 44.746 | 86.987 | 1.00 | 49.64 | C |
| ATOM | 1375 | OD1 | ASP | A | 496 | 18.747 | 45.446 | 87.528 | 1.00 | 49.69 | O |
| ATOM | 1376 | OD2 | ASP | A | 496 | 20.349 | 43.938 | 87.612 | 1.00 | 48.95 | O |
| ATOM | 1377 | N | PRO | A | 497 | 19.089 | 48.220 | 85.102 | 1.00 | 49.61 | N |
| ATOM | 1378 | CA | PRO | A | 497 | 18.498 | 49.306 | 84.330 | 1.00 | 49.37 | C |
| ATOM | 1379 | C | PRO | A | 497 | 19.569 | 50.317 | 83.869 | 1.00 | 49.09 | C |
| ATOM | 1380 | O | PRO | A | 497 | 19.712 | 51.401 | 84.445 | 1.00 | 49.14 | O |
| ATOM | 1381 | CB | PRO | A | 497 | 17.493 | 49.935 | 85.313 | 1.00 | 49.36 | C |
| ATOM | 1382 | CG | PRO | A | 497 | 17.737 | 49.253 | 86.664 | 1.00 | 49.79 | C |
| ATOM | 1383 | CD | PRO | A | 497 | 19.037 | 48.515 | 86.543 | 1.00 | 49.76 | C |
| ATOM | 1384 | N | VAL | A | 498 | 20.314 | 49.929 | 82.838 | 1.00 | 48.56 | N |
| ATOM | 1385 | CA | VAL | A | 498 | 21.325 | 50.767 | 82.184 | 1.00 | 48.25 | C |
| ATOM | 1386 | C | VAL | A | 498 | 20.712 | 51.380 | 80.912 | 1.00 | 47.87 | C |
| ATOM | 1387 | O | VAL | A | 498 | 19.615 | 50.957 | 80.541 | 1.00 | 47.91 | O |
| ATOM | 1388 | CB | VAL | A | 498 | 22.510 | 49.865 | 81.757 | 1.00 | 48.37 | C |
| ATOM | 1389 | CG1 | VAL | A | 498 | 23.269 | 49.381 | 82.967 | 1.00 | 48.71 | C |
| ATOM | 1390 | CG2 | VAL | A | 498 | 22.011 | 48.645 | 80.973 | 1.00 | 47.94 | C |
| ATOM | 1391 | N | LYS | A | 499 | 21.303 | 52.394 | 80.251 | 1.00 | 47.45 | N |
| ATOM | 1392 | CA | LYS | A | 499 | 21.910 | 53.668 | 80.760 | 1.00 | 46.98 | C |
| ATOM | 1393 | C | LYS | A | 499 | 22.723 | 54.484 | 79.707 | 1.00 | 46.12 | C |
| ATOM | 1394 | O | LYS | A | 499 | 22.172 | 55.438 | 79.153 | 1.00 | 46.15 | O |
| ATOM | 1395 | CB | LYS | A | 499 | 22.474 | 53.650 | 82.189 | 1.00 | 47.26 | C |
| ATOM | 1396 | CG | LYS | A | 499 | 21.515 | 54.396 | 83.127 | 1.00 | 47.49 | C |
| ATOM | 1397 | CD | LYS | A | 499 | 21.848 | 54.249 | 84.613 | 1.00 | 47.72 | C |
| ATOM | 1398 | CE | LYS | A | 499 | 20.859 | 55.117 | 85.430 | 1.00 | 48.69 | C |
| ATOM | 1399 | NZ | LYS | A | 499 | 20.973 | 54.934 | 86.913 | 1.00 | 49.29 | N |
| ATOM | 1400 | N | PRO | A | 500 | 24.017 | 54.167 | 79.459 | 1.00 | 45.38 | N |
| ATOM | 1401 | CA | PRO | A | 500 | 24.596 | 54.413 | 78.118 | 1.00 | 44.83 | C |
| ATOM | 1402 | C | PRO | A | 500 | 24.112 | 53.359 | 77.105 | 1.00 | 44.17 | C |
| ATOM | 1403 | O | PRO | A | 500 | 22.981 | 52.885 | 77.227 | 1.00 | 44.27 | O |
| ATOM | 1404 | CB | PRO | A | 500 | 26.113 | 54.292 | 78.350 | 1.00 | 44.82 | C |
| ATOM | 1405 | CG | PRO | A | 500 | 26.300 | 54.334 | 79.837 | 1.00 | 45.03 | C |
| ATOM | 1406 | CD | PRO | A | 500 | 25.053 | 53.724 | 80.401 | 1.00 | 45.46 | C |
| ATOM | 1407 | N | ALA | A | 501 | 24.932 | 52.988 | 76.119 | 1.00 | 43.29 | N |
| ATOM | 1408 | CA | ALA | A | 501 | 24.521 | 51.941 | 75.158 | 1.00 | 42.43 | C |
| ATOM | 1409 | C | ALA | A | 501 | 24.677 | 50.525 | 75.755 | 1.00 | 41.67 | C |
| ATOM | 1410 | O | ALA | A | 501 | 25.008 | 49.553 | 75.066 | 1.00 | 41.39 | O |
| ATOM | 1411 | CB | ALA | A | 501 | 25.249 | 52.092 | 73.819 | 1.00 | 42.25 | C |
| ATOM | 1412 | N | TYR | A | 502 | 24.425 | 50.453 | 77.062 | 1.00 | 40.73 | N |
| ATOM | 1413 | CA | TYR | A | 502 | 24.354 | 49.214 | 77.846 | 1.00 | 39.80 | C |
| ATOM | 1414 | C | TYR | A | 502 | 25.704 | 48.520 | 78.076 | 1.00 | 39.34 | C |
| ATOM | 1415 | O | TYR | A | 502 | 25.747 | 47.387 | 78.555 | 1.00 | 39.10 | O |
| ATOM | 1416 | CB | TYR | A | 502 | 23.271 | 48.259 | 77.299 | 1.00 | 39.59 | C |
| ATOM | 1417 | CG | TYR | A | 502 | 21.994 | 48.963 | 76.867 | 1.00 | 39.08 | C |
| ATOM | 1418 | CD1 | TYR | A | 502 | 21.276 | 49.763 | 77.757 | 1.00 | 39.08 | C |
| ATOM | 1419 | CD2 | TYR | A | 502 | 21.512 | 48.838 | 75.565 | 1.00 | 38.95 | C |
| ATOM | 1420 | CE1 | TYR | A | 502 | 20.115 | 50.419 | 77.364 | 1.00 | 38.94 | C |
| ATOM | 1421 | CE2 | TYR | A | 502 | 20.346 | 49.486 | 75.162 | 1.00 | 38.78 | C |
| ATOM | 1422 | CZ | TYR | A | 502 | 19.655 | 50.275 | 76.067 | 1.00 | 39.16 | C |
| ATOM | 1423 | OH | TYR | A | 502 | 18.501 | 50.921 | 75.682 | 1.00 | 39.14 | O |
| ATOM | 1424 | N | SER | A | 503 | 26.798 | 49.221 | 77.770 | 1.00 | 38.77 | N |
| ATOM | 1425 | CA | SER | A | 503 | 28.149 | 48.731 | 78.064 | 1.00 | 38.23 | C |
| ATOM | 1426 | C | SER | A | 503 | 28.399 | 48.607 | 79.570 | 1.00 | 37.99 | C |
| ATOM | 1427 | O | SER | A | 503 | 29.287 | 47.867 | 80.004 | 1.00 | 37.96 | O |
| ATOM | 1428 | CB | SER | A | 503 | 29.208 | 49.639 | 77.432 | 1.00 | 38.28 | C |
| ATOM | 1429 | OG | SER | A | 503 | 29.057 | 50.983 | 77.853 | 1.00 | 38.21 | O |
| ATOM | 1430 | N | ASP | A | 504 | 27.599 | 49.328 | 80.353 | 1.00 | 37.38 | N |
| ATOM | 1431 | CA | ASP | A | 504 | 27.692 | 49.314 | 81.810 | 1.00 | 36.90 | C |
| ATOM | 1432 | C | ASP | A | 504 | 26.814 | 48.245 | 82.482 | 1.00 | 36.55 | C |
| ATOM | 1433 | O | ASP | A | 504 | 26.757 | 48.179 | 83.716 | 1.00 | 36.41 | O |
| ATOM | 1434 | CB | ASP | A | 504 | 27.359 | 50.703 | 82.370 | 1.00 | 36.92 | C |
| ATOM | 1435 | CG | ASP | A | 504 | 25.976 | 51.182 | 81.967 | 1.00 | 36.85 | C |
| ATOM | 1436 | OD1 | ASP | A | 504 | 25.591 | 50.997 | 80.791 | 1.00 | 35.69 | O |
| ATOM | 1437 | OD2 | ASP | A | 504 | 25.274 | 51.754 | 82.826 | 1.00 | 37.49 | O |
| ATOM | 1438 | N | LYS | A | 505 | 26.131 | 47.421 | 81.683 | 1.00 | 36.02 | N |
| ATOM | 1439 | CA | LYS | A | 505 | 25.350 | 46.302 | 82.224 | 1.00 | 35.44 | C |
| ATOM | 1440 | C | LYS | A | 505 | 26.267 | 45.331 | 82.960 | 1.00 | 35.34 | C |
| ATOM | 1441 | O | LYS | A | 505 | 27.318 | 44.952 | 82.447 | 1.00 | 35.26 | O |
| ATOM | 1442 | CB | LYS | A | 505 | 24.561 | 45.573 | 81.127 | 1.00 | 35.29 | C |
| ATOM | 1443 | CG | LYS | A | 505 | 23.566 | 44.543 | 81.664 | 1.00 | 34.88 | C |
| ATOM | 1444 | CD | LYS | A | 505 | 22.717 | 43.927 | 80.562 | 1.00 | 34.92 | C |
| ATOM | 1445 | CE | LYS | A | 505 | 21.958 | 42.698 | 81.052 | 1.00 | 34.21 | C |
| ATOM | 1446 | NZ | LYS | A | 505 | 20.795 | 43.023 | 81.926 | 1.00 | 34.41 | N |
| ATOM | 1447 | N | GLY | A | 506 | 25.873 | 44.954 | 84.171 | 1.00 | 35.37 | N |
| ATOM | 1448 | CA | GLY | A | 506 | 26.664 | 44.038 | 84.991 | 1.00 | 35.52 | C |
| ATOM | 1449 | C | GLY | A | 506 | 27.775 | 44.701 | 85.794 | 1.00 | 35.69 | C |
| ATOM | 1450 | O | GLY | A | 506 | 28.508 | 44.025 | 86.522 | 1.00 | 35.44 | O |
| ATOM | 1451 | N | ASP | A | 507 | 27.912 | 46.018 | 85.652 | 1.00 | 35.99 | N |
| ATOM | 1452 | CA | ASP | A | 507 | 28.857 | 46.785 | 86.458 | 1.00 | 36.55 | C |
| ATOM | 1453 | C | ASP | A | 507 | 28.509 | 46.612 | 87.927 | 1.00 | 37.00 | C |
| ATOM | 1454 | O | ASP | A | 507 | 27.330 | 46.637 | 88.301 | 1.00 | 37.00 | O |
| ATOM | 1455 | CB | ASP | A | 507 | 28.834 | 48.273 | 86.083 | 1.00 | 36.33 | C |
| ATOM | 1456 | CG | ASP | A | 507 | 29.752 | 48.603 | 84.911 | 1.00 | 36.35 | C |
| ATOM | 1457 | OD1 | ASP | A | 507 | 30.431 | 47.691 | 84.387 | 1.00 | 35.90 | O |
| ATOM | 1458 | OD2 | ASP | A | 507 | 29.794 | 49.788 | 84.509 | 1.00 | 36.31 | O |
| ATOM | 1459 | N | LEU | A | 508 | 29.531 | 46.415 | 88.752 | 1.00 | 37.44 | N |
| ATOM | 1460 | CA | LEU | A | 508 | 29.306 | 46.216 | 90.177 | 1.00 | 38.11 | C |
| ATOM | 1461 | C | LEU | A | 508 | 29.794 | 47.389 | 91.030 | 1.00 | 38.59 | C |
| ATOM | 1462 | O | LEU | A | 508 | 30.950 | 47.813 | 90.937 | 1.00 | 38.69 | O |
| ATOM | 1463 | CB | LEU | A | 508 | 29.901 | 44.883 | 90.648 | 1.00 | 37.85 | C |
| ATOM | 1464 | CG | LEU | A | 508 | 29.372 | 44.443 | 92.019 | 1.00 | 37.85 | C |
| ATOM | 1465 | CD1 | LEU | A | 508 | 28.942 | 42.996 | 92.002 | 1.00 | 36.87 | C |
| ATOM | 1466 | CD2 | LEU | A | 508 | 30.382 | 44.710 | 93.131 | 1.00 | 37.66 | C |
| ATOM | 1467 | N | TYR | A | 509 | 28.884 | 47.903 | 91.851 | 1.00 | 39.10 | N |
| ATOM | 1468 | CA | TYR | A | 509 | 29.150 | 49.011 | 92.755 | 1.00 | 39.80 | C |
| ATOM | 1469 | C | TYR | A | 509 | 29.136 | 48.515 | 94.201 | 1.00 | 40.29 | C |
| ATOM | 1470 | O | TYR | A | 509 | 28.437 | 47.553 | 94.515 | 1.00 | 40.22 | O |
| ATOM | 1471 | CB | TYR | A | 509 | 28.076 | 50.088 | 92.570 | 1.00 | 39.71 | C |
| ATOM | 1472 | CG | TYR | A | 509 | 28.226 | 50.922 | 91.312 | 1.00 | 39.89 | C |
| ATOM | 1473 | CD1 | TYR | A | 509 | 28.698 | 52.234 | 91.379 | 1.00 | 39.75 | C |
| ATOM | 1474 | CD2 | TYR | A | 509 | 27.890 | 50.407 | 90.054 | 1.00 | 39.64 | C |
| ATOM | 1475 | CE1 | TYR | A | 509 | 28.840 | 53.010 | 90.230 | 1.00 | 39.56 | C |
| ATOM | 1476 | CE2 | TYR | A | 509 | 28.030 | 51.174 | 88.897 | 1.00 | 38.87 | C |
| ATOM | 1477 | CZ | TYR | A | 509 | 28.505 | 52.473 | 88.995 | 1.00 | 39.71 | C |
| ATOM | 1478 | OH | TYR | A | 509 | 28.643 | 53.244 | 87.865 | 1.00 | 39.86 | O |
| ATOM | 1479 | N | LYS | A | 510 | 29.919 | 49.159 | 95.070 | 1.00 | 41.14 | N |
| ATOM | 1480 | CA | LYS | A | 510 | 29.775 | 48.972 | 96.522 | 1.00 | 41.86 | C |
| ATOM | 1481 | C | LYS | A | 510 | 29.113 | 50.190 | 97.150 | 1.00 | 42.35 | C |
| ATOM | 1482 | O | LYS | A | 510 | 28.266 | 50.061 | 98.026 | 1.00 | 42.45 | O |
| ATOM | 1483 | CB | LYS | A | 510 | 31.108 | 48.694 | 97.217 | 1.00 | 41.86 | C |
| ATOM | 1484 | CG | LYS | A | 510 | 30.945 | 48.005 | 98.574 | 1.00 | 42.23 | C |
| ATOM | 1485 | CD | LYS | A | 510 | 32.194 | 48.145 | 99.433 | 1.00 | 43.27 | C |
| ATOM | 1486 | CE | LYS | A | 510 | 32.377 | 46.959 | 100.382 | 1.00 | 43.78 | C |
| ATOM | 1487 | NZ | LYS | A | 510 | 31.374 | 46.895 | 101.486 | 1.00 | 44.22 | N |
| ATOM | 1488 | N | GLY | A | 511 | 29.507 | 51.378 | 96.712 | 1.00 | 42.80 | N |
| ATOM | 1489 | CA | GLY | A | 511 | 28.849 | 52.593 | 97.172 | 1.00 | 43.41 | C |
| ATOM | 1490 | C | GLY | A | 511 | 28.359 | 53.353 | 95.968 | 1.00 | 43.80 | C |
| ATOM | 1491 | O | GLY | A | 511 | 27.600 | 52.822 | 95.150 | 1.00 | 43.93 | O |
| ATOM | 1492 | N | ASN | A | 512 | 28.801 | 54.600 | 95.866 | 1.00 | 44.06 | N |
| ATOM | 1493 | CA | ASN | A | 512 | 28.640 | 55.381 | 94.650 | 1.00 | 44.35 | C |
| ATOM | 1494 | C | ASN | A | 512 | 29.834 | 55.156 | 93.719 | 1.00 | 44.19 | C |
| ATOM | 1495 | O | ASN | A | 512 | 29.887 | 55.697 | 92.608 | 1.00 | 44.04 | O |
| ATOM | 1496 | CB | ASN | A | 512 | 28.495 | 56.866 | 94.996 | 1.00 | 44.59 | C |
| ATOM | 1497 | CG | ASN | A | 512 | 27.242 | 57.156 | 95.804 | 1.00 | 45.35 | C |
| ATOM | 1498 | OD1 | ASN | A | 512 | 26.153 | 56.673 | 95.482 | 1.00 | 46.76 | O |
| ATOM | 1499 | ND2 | ASN | A | 512 | 27.389 | 57.953 | 96.856 | 1.00 | 45.71 | N |
| ATOM | 1500 | N | GLN | A | 513 | 30.786 | 54.352 | 94.194 | 1.00 | 44.07 | N |
| ATOM | 1501 | CA | GLN | A | 513 | 32.001 | 54.027 | 93.455 | 1.00 | 44.17 | C |
| ATOM | 1502 | C | GLN | A | 513 | 31.863 | 52.675 | 92.761 | 1.00 | 43.91 | C |
| ATOM | 1503 | O | GLN | A | 513 | 31.132 | 51.799 | 93.234 | 1.00 | 43.90 | O |
| ATOM | 1504 | CB | GLN | A | 513 | 33.218 | 54.018 | 94.387 | 1.00 | 44.45 | C |
| ATOM | 1505 | CG | GLN | A | 513 | 33.240 | 52.863 | 95.395 | 1.00 | 45.29 | C |
| ATOM | 1506 | CD | GLN | A | 513 | 34.648 | 52.456 | 95.814 | 1.00 | 47.13 | C |
| ATOM | 1507 | OE1 | GLN | A | 513 | 35.643 | 52.826 | 95.176 | 1.00 | 47.20 | O |
| ATOM | 1508 | NE2 | GLN | A | 513 | 34.736 | 51.677 | 96.890 | 1.00 | 47.65 | N |
| ATOM | 1509 | N | LEU | A | 514 | 32.578 | 52.511 | 91.647 | 1.00 | 43.53 | N |
| ATOM | 1510 | CA | LEU | A | 514 | 32.462 | 51.313 | 90.814 | 1.00 | 43.01 | C |
| ATOM | 1511 | C | LEU | A | 514 | 33.038 | 50.078 | 91.522 | 1.00 | 42.63 | C |
| ATOM | 1512 | O | LEU | A | 514 | 32.561 | 49.712 | 92.605 | 1.00 | 42.80 | O |
| ATOM | 1513 | CB | LEU | A | 514 | 33.092 | 51.555 | 89.433 | 1.00 | 43.01 | C |
| ATOM | 1514 | CG | LEU | A | 514 | 32.745 | 50.683 | 88.215 | 1.00 | 43.12 | C |
| ATOM | 1515 | CD1 | LEU | A | 514 | 31.290 | 50.238 | 88.195 | 1.00 | 42.92 | C |
| ATOM | 1516 | CD2 | LEU | A | 514 | 33.095 | 51.421 | 86.924 | 1.00 | 43.05 | C |
| ATOM | 1517 | N | LEU | A | 515 | 34.043 | 49.449 | 90.911 | 1.00 | 41.83 | N |
| ATOM | 1518 | CA | LEU | A | 515 | 34.692 | 48.216 | 91.394 | 1.00 | 41.05 | C |
| ATOM | 1519 | C | LEU | A | 515 | 35.069 | 47.341 | 90.214 | 1.00 | 40.26 | C |
| ATOM | 1520 | O | LEU | A | 515 | 36.246 | 47.206 | 89.887 | 1.00 | 40.28 | O |
| ATOM | 1521 | CB | LEU | A | 515 | 33.824 | 47.410 | 92.373 | 1.00 | 41.19 | C |
| ATOM | 1522 | CG | LEU | A | 515 | 34.149 | 47.467 | 93.871 | 1.00 | 41.69 | C |
| ATOM | 1523 | CD1 | LEU | A | 515 | 32.981 | 46.934 | 94.679 | 1.00 | 42.03 | C |
| ATOM | 1524 | CD2 | LEU | A | 515 | 35.417 | 46.688 | 94.197 | 1.00 | 42.18 | C |
| ATOM | 1525 | N | GLY | A | 516 | 34.061 | 46.757 | 89.571 | 1.00 | 39.38 | N |
| ATOM | 1526 | CA | GLY | A | 516 | 34.281 | 45.882 | 88.424 | 1.00 | 37.96 | C |
| ATOM | 1527 | C | GLY | A | 516 | 33.013 | 45.540 | 87.671 | 1.00 | 36.96 | C |
| ATOM | 1528 | O | GLY | A | 516 | 32.097 | 46.362 | 87.562 | 1.00 | 37.01 | O |
| ATOM | 1529 | N | ASN | A | 517 | 32.960 | 44.317 | 87.155 | 1.00 | 35.84 | N |
| ATOM | 1530 | CA | ASN | A | 517 | 31.839 | 43.868 | 86.335 | 1.00 | 34.59 | C |
| ATOM | 1531 | C | ASN | A | 517 | 31.652 | 42.356 | 86.448 | 1.00 | 34.17 | C |
| ATOM | 1532 | O | ASN | A | 517 | 32.616 | 41.593 | 86.343 | 1.00 | 33.85 | O |
| ATOM | 1533 | CB | ASN | A | 517 | 32.046 | 44.303 | 84.878 | 1.00 | 34.41 | C |
| ATOM | 1534 | CG | ASN | A | 517 | 30.852 | 44.008 | 83.996 | 1.00 | 32.96 | C |
| ATOM | 1535 | OD1 | ASN | A | 517 | 30.587 | 42.859 | 83.658 | 1.00 | 31.49 | O |
| ATOM | 1536 | ND2 | ASN | A | 517 | 30.138 | 45.054 | 83.598 | 1.00 | 31.54 | N |
| ATOM | 1537 | N | ILE | A | 518 | 30.404 | 41.936 | 86.652 | 1.00 | 33.65 | N |
| ATOM | 1538 | CA | ILE | A | 518 | 30.083 | 40.531 | 86.941 | 1.00 | 33.11 | C |
| ATOM | 1539 | C | ILE | A | 518 | 30.291 | 39.570 | 85.766 | 1.00 | 32.83 | C |
| ATOM | 1540 | O | ILE | A | 518 | 30.252 | 38.355 | 85.945 | 1.00 | 32.66 | O |
| ATOM | 1541 | CB | ILE | A | 518 | 28.650 | 40.371 | 87.514 | 1.00 | 33.12 | C |
| ATOM | 1542 | CG1 | ILE | A | 518 | 27.605 | 40.844 | 86.496 | 1.00 | 33.15 | C |
| ATOM | 1543 | CG2 | ILE | A | 518 | 28.529 | 41.109 | 88.837 | 1.00 | 32.57 | C |
| ATOM | 1544 | CD1 | ILE | A | 518 | 26.184 | 40.446 | 86.812 | 1.00 | 32.82 | C |
| ATOM | 1545 | N | TYR | A | 519 | 30.509 | 40.116 | 84.573 | 1.00 | 32.73 | N |
| ATOM | 1546 | CA | TYR | A | 519 | 30.821 | 39.295 | 83.399 | 1.00 | 32.71 | C |
| ATOM | 1547 | C | TYR | A | 519 | 32.336 | 39.140 | 83.179 | 1.00 | 32.89 | C |
| ATOM | 1548 | O | TYR | A | 519 | 32.762 | 38.473 | 82.232 | 1.00 | 33.04 | O |
| ATOM | 1549 | CB | TYR | A | 519 | 30.151 | 39.848 | 82.129 | 1.00 | 32.15 | C |
| ATOM | 1550 | CG | TYR | A | 519 | 28.693 | 40.221 | 82.270 | 1.00 | 31.53 | C |
| ATOM | 1551 | CD1 | TYR | A | 519 | 27.779 | 39.362 | 82.886 | 1.00 | 30.97 | C |
| ATOM | 1552 | CD2 | TYR | A | 519 | 28.220 | 41.430 | 81.768 | 1.00 | 31.22 | C |
| ATOM | 1553 | CE1 | TYR | A | 519 | 26.432 | 39.709 | 83.007 | 1.00 | 30.51 | C |
| ATOM | 1554 | CE2 | TYR | A | 519 | 26.878 | 41.786 | 81.886 | 1.00 | 30.74 | C |
| ATOM | 1555 | CZ | TYR | A | 519 | 25.993 | 40.923 | 82.506 | 1.00 | 31.02 | C |
| ATOM | 1556 | OH | TYR | A | 519 | 24.671 | 41.285 | 82.619 | 1.00 | 31.14 | O |
| ATOM | 1557 | N | PHE | A | 520 | 33.136 | 39.760 | 84.046 | 1.00 | 33.18 | N |
| ATOM | 1558 | CA | PHE | A | 520 | 34.597 | 39.650 | 83.991 | 1.00 | 33.68 | C |
| ATOM | 1559 | C | PHE | A | 520 | 35.022 | 38.331 | 84.623 | 1.00 | 33.97 | C |
| ATOM | 1560 | O | PHE | A | 520 | 34.493 | 37.941 | 85.664 | 1.00 | 34.03 | O |
| ATOM | 1561 | CB | PHE | A | 520 | 35.262 | 40.828 | 84.718 | 1.00 | 33.76 | C |
| ATOM | 1562 | CG | PHE | A | 520 | 35.131 | 42.163 | 84.004 | 1.00 | 34.02 | C |
| ATOM | 1563 | CD1 | PHE | A | 520 | 34.382 | 42.294 | 82.834 | 1.00 | 33.80 | C |
| ATOM | 1564 | CD2 | PHE | A | 520 | 35.738 | 43.300 | 84.531 | 1.00 | 34.57 | C |
| ATOM | 1565 | CE1 | PHE | A | 520 | 34.262 | 43.523 | 82.192 | 1.00 | 34.23 | C |
| ATOM | 1566 | CE2 | PHE | A | 520 | 35.623 | 44.538 | 83.899 | 1.00 | 34.73 | C |
| ATOM | 1567 | CZ | PHE | A | 520 | 34.886 | 44.649 | 82.724 | 1.00 | 34.63 | C |
| ATOM | 1568 | N | THR | A | 521 | 35.968 | 37.649 | 83.986 | 1.00 | 34.46 | N |
| ATOM | 1569 | CA | THR | A | 521 | 36.383 | 36.302 | 84.392 | 1.00 | 35.27 | C |
| ATOM | 1570 | C | THR | A | 521 | 37.821 | 36.273 | 84.924 | 1.00 | 35.45 | C |
| ATOM | 1571 | O | THR | A | 521 | 38.151 | 35.467 | 85.794 | 1.00 | 35.52 | O |
| ATOM | 1572 | CB | THR | A | 521 | 36.243 | 35.309 | 83.212 | 1.00 | 35.33 | C |
| ATOM | 1573 | OG1 | THR | A | 521 | 34.940 | 35.447 | 82.632 | 1.00 | 36.70 | O |
| ATOM | 1574 | CG2 | THR | A | 521 | 36.421 | 33.862 | 83.668 | 1.00 | 35.85 | C |
| ATOM | 1575 | N | THR | A | 522 | 38.658 | 37.170 | 84.407 | 1.00 | 35.73 | N |
| ATOM | 1576 | CA | THR | A | 522 | 40.078 | 37.200 | 84.738 | 1.00 | 35.92 | C |
| ATOM | 1577 | C | THR | A | 522 | 40.465 | 38.536 | 85.377 | 1.00 | 36.02 | C |
| ATOM | 1578 | O | THR | A | 522 | 39.880 | 39.574 | 85.055 | 1.00 | 35.77 | O |
| ATOM | 1579 | CB | THR | A | 522 | 40.932 | 36.948 | 83.471 | 1.00 | 35.96 | C |
| ATOM | 1580 | OG1 | THR | A | 522 | 40.454 | 35.775 | 82.801 | 1.00 | 35.53 | O |
| ATOM | 1581 | CG2 | THR | A | 522 | 42.395 | 36.745 | 83.825 | 1.00 | 36.58 | C |
| ATOM | 1582 | N | ASN | A | 523 | 41.439 | 38.488 | 86.291 | 1.00 | 36.10 | N |
| ATOM | 1583 | CA | ASN | A | 523 | 42.035 | 39.680 | 86.918 | 1.00 | 36.32 | C |
| ATOM | 1584 | C | ASN | A | 523 | 40.999 | 40.682 | 87.426 | 1.00 | 36.45 | C |
| ATOM | 1585 | O | ASN | A | 523 | 41.085 | 41.884 | 87.152 | 1.00 | 36.54 | O |
| ATOM | 1586 | CB | ASN | A | 523 | 43.037 | 40.359 | 85.968 | 1.00 | 36.25 | C |
| ATOM | 1587 | CG | ASN | A | 523 | 44.072 | 39.385 | 85.408 | 1.00 | 36.56 | C |
| ATOM | 1588 | OD1 | ASN | A | 523 | 44.394 | 38.371 | 86.031 | 1.00 | 37.31 | O |
| ATOM | 1589 | ND2 | ASN | A | 523 | 44.592 | 39.691 | 84.224 | 1.00 | 36.35 | N |
| ATOM | 1590 | N | LYS | A | 524 | 40.031 | 40.168 | 88.177 | 1.00 | 36.46 | N |
| ATOM | 1591 | CA | LYS | A | 524 | 38.869 | 40.938 | 88.588 | 1.00 | 36.48 | C |
| ATOM | 1592 | C | LYS | A | 524 | 39.200 | 41.961 | 89.661 | 1.00 | 36.87 | C |
| ATOM | 1593 | O | LYS | A | 524 | 39.984 | 41.693 | 90.570 | 1.00 | 36.78 | O |
| ATOM | 1594 | CB | LYS | A | 524 | 37.758 | 40.006 | 89.074 | 1.00 | 36.52 | C |
| ATOM | 1595 | CG | LYS | A | 524 | 37.164 | 39.116 | 87.979 | 1.00 | 36.16 | C |
| ATOM | 1596 | CD | LYS | A | 524 | 36.205 | 38.074 | 88.556 | 1.00 | 35.90 | C |
| ATOM | 1597 | CE | LYS | A | 524 | 34.956 | 38.714 | 89.151 | 1.00 | 34.47 | C |
| ATOM | 1598 | NZ | LYS | A | 524 | 34.193 | 39.535 | 88.162 | 1.00 | 33.84 | N |
| ATOM | 1599 | N | THR | A | 525 | 38.587 | 43.134 | 89.533 | 1.00 | 37.24 | N |
| ATOM | 1600 | CA | THR | A | 525 | 38.709 | 44.212 | 90.511 | 1.00 | 37.48 | C |
| ATOM | 1601 | C | THR | A | 525 | 37.523 | 44.212 | 91.494 | 1.00 | 37.34 | C |
| ATOM | 1602 | O | THR | A | 525 | 37.500 | 44.989 | 92.446 | 1.00 | 37.42 | O |
| ATOM | 1603 | CB | THR | A | 525 | 38.836 | 45.580 | 89.794 | 1.00 | 37.67 | C |
| ATOM | 1604 | OG1 | THR | A | 525 | 39.767 | 45.466 | 88.713 | 1.00 | 37.73 | O |
| ATOM | 1605 | CG2 | THR | A | 525 | 39.322 | 46.677 | 90.755 | 1.00 | 38.48 | C |
| ATOM | 1606 | N | SER | A | 526 | 36.549 | 43.332 | 91.255 | 1.00 | 37.27 | N |
| ATOM | 1607 | CA | SER | A | 526 | 35.387 | 43.159 | 92.135 | 1.00 | 37.12 | C |
| ATOM | 1608 | C | SER | A | 526 | 35.306 | 41.699 | 92.581 | 1.00 | 36.73 | C |
| ATOM | 1609 | O | SER | A | 526 | 35.842 | 40.827 | 91.903 | 1.00 | 37.17 | O |
| ATOM | 1610 | CB | SER | A | 526 | 34.092 | 43.565 | 91.415 | 1.00 | 37.28 | C |
| ATOM | 1611 | OG | SER | A | 526 | 33.626 | 42.535 | 90.551 | 1.00 | 37.83 | O |
| ATOM | 1612 | N | PRO | A | 527 | 34.641 | 41.422 | 93.718 | 1.00 | 36.38 | N |
| ATOM | 1613 | CA | PRO | A | 527 | 34.566 | 40.028 | 94.177 | 1.00 | 36.04 | C |
| ATOM | 1614 | C | PRO | A | 527 | 33.504 | 39.141 | 93.499 | 1.00 | 35.63 | C |
| ATOM | 1615 | O | PRO | A | 527 | 33.489 | 37.931 | 93.733 | 1.00 | 35.58 | O |
| ATOM | 1616 | CB | PRO | A | 527 | 34.263 | 40.172 | 95.673 | 1.00 | 35.94 | C |
| ATOM | 1617 | CG | PRO | A | 527 | 33.524 | 41.453 | 95.789 | 1.00 | 36.26 | C |
| ATOM | 1618 | CD | PRO | A | 527 | 33.962 | 42.347 | 94.648 | 1.00 | 36.37 | C |
| ATOM | 1619 | N | PHE | A | 528 | 32.643 | 39.720 | 92.665 | 1.00 | 35.27 | N |
| ATOM | 1620 | CA | PHE | A | 528 | 31.503 | 38.970 | 92.117 | 1.00 | 34.92 | C |
| ATOM | 1621 | C | PHE | A | 528 | 31.518 | 38.714 | 90.612 | 1.00 | 34.47 | C |
| ATOM | 1622 | O | PHE | A | 528 | 31.792 | 39.612 | 89.815 | 1.00 | 34.56 | O |
| ATOM | 1623 | CB | PHE | A | 528 | 30.181 | 39.635 | 92.500 | 1.00 | 34.95 | C |
| ATOM | 1624 | CG | PHE | A | 528 | 29.921 | 39.660 | 93.972 | 1.00 | 35.46 | C |
| ATOM | 1625 | CD1 | PHE | A | 528 | 29.482 | 38.518 | 94.633 | 1.00 | 35.46 | C |
| ATOM | 1626 | CD2 | PHE | A | 528 | 30.110 | 40.829 | 94.702 | 1.00 | 35.96 | C |
| ATOM | 1627 | CE1 | PHE | A | 528 | 29.239 | 38.538 | 95.998 | 1.00 | 35.79 | C |
| ATOM | 1628 | CE2 | PHE | A | 528 | 29.871 | 40.859 | 96.070 | 1.00 | 35.87 | C |
| ATOM | 1629 | CZ | PHE | A | 528 | 29.433 | 39.713 | 96.720 | 1.00 | 35.73 | C |
| ATOM | 1630 | N | ARG | A | 529 | 31.197 | 37.476 | 90.247 | 1.00 | 33.94 | N |
| ATOM | 1631 | CA | ARG | A | 529 | 30.994 | 37.083 | 88.854 | 1.00 | 33.18 | C |
| ATOM | 1632 | C | ARG | A | 529 | 29.773 | 36.169 | 88.727 | 1.00 | 32.69 | C |
| ATOM | 1633 | O | ARG | A | 529 | 29.367 | 35.524 | 89.695 | 1.00 | 32.65 | O |
| ATOM | 1634 | CB | ARG | A | 529 | 32.236 | 36.379 | 88.298 | 1.00 | 33.17 | C |
| ATOM | 1635 | CG | ARG | A | 529 | 32.566 | 35.044 | 88.954 | 1.00 | 33.25 | C |
| ATOM | 1636 | CD | ARG | A | 529 | 33.445 | 34.198 | 88.048 | 1.00 | 34.49 | C |
| ATOM | 1637 | NE | ARG | A | 529 | 33.723 | 32.882 | 88.624 | 1.00 | 35.65 | N |
| ATOM | 1638 | CZ | ARG | A | 529 | 32.970 | 31.796 | 88.453 | 1.00 | 36.54 | C |
| ATOM | 1639 | NH1 | ARG | A | 529 | 31.865 | 31.838 | 87.711 | 1.00 | 35.55 | N |
| ATOM | 1640 | NH2 | ARG | A | 529 | 33.328 | 30.653 | 89.030 | 1.00 | 37.41 | N |
| ATOM | 1641 | N | ILE | A | 530 | 29.194 | 36.115 | 87.531 | 1.00 | 31.91 | N |
| ATOM | 1642 | CA | ILE | A | 530 | 28.122 | 35.160 | 87.245 | 1.00 | 31.11 | C |
| ATOM | 1643 | C | ILE | A | 530 | 28.706 | 33.773 | 86.990 | 1.00 | 30.48 | C |
| ATOM | 1644 | O | ILE | A | 530 | 29.925 | 33.589 | 87.033 | 1.00 | 30.43 | O |
| ATOM | 1645 | CB | ILE | A | 530 | 27.245 | 35.596 | 86.043 | 1.00 | 31.17 | C |
| ATOM | 1646 | CG1 | ILE | A | 530 | 28.090 | 35.765 | 84.770 | 1.00 | 31.17 | C |
| ATOM | 1647 | CG2 | ILE | A | 530 | 26.482 | 36.871 | 86.386 | 1.00 | 31.14 | C |
| ATOM | 1648 | CD1 | ILE | A | 530 | 27.279 | 35.861 | 83.468 | 1.00 | 31.03 | C |
| ATOM | 1649 | N | ALA | A | 531 | 27.830 | 32.807 | 86.734 | 1.00 | 29.68 | N |
| ATOM | 1650 | CA | ALA | A | 531 | 28.236 | 31.450 | 86.374 | 1.00 | 28.82 | C |
| ATOM | 1651 | C | ALA | A | 531 | 28.822 | 31.394 | 84.962 | 1.00 | 28.17 | C |
| ATOM | 1652 | O | ALA | A | 531 | 28.340 | 32.071 | 84.054 | 1.00 | 27.87 | O |
| ATOM | 1653 | CB | ALA | A | 531 | 27.054 | 30.498 | 86.490 | 1.00 | 28.75 | C |
| ATOM | 1654 | N | LYS | A | 532 | 29.858 | 30.576 | 84.794 | 1.00 | 27.59 | N |
| ATOM | 1655 | CA | LYS | A | 532 | 30.506 | 30.387 | 83.503 | 1.00 | 27.27 | C |
| ATOM | 1656 | C | LYS | A | 532 | 29.610 | 29.518 | 82.620 | 1.00 | 26.30 | C |
| ATOM | 1657 | O | LYS | A | 532 | 29.665 | 28.285 | 82.655 | 1.00 | 26.00 | O |
| ATOM | 1658 | CB | LYS | A | 532 | 31.889 | 29.763 | 83.698 | 1.00 | 27.16 | C |
| ATOM | 1659 | CG | LYS | A | 532 | 32.848 | 29.905 | 82.515 | 1.00 | 27.98 | C |
| ATOM | 1660 | CD | LYS | A | 532 | 34.233 | 29.327 | 82.882 | 1.00 | 28.75 | C |
| ATOM | 1661 | CE | LYS | A | 532 | 35.103 | 29.054 | 81.650 | 1.00 | 31.48 | C |
| ATOM | 1662 | NZ | LYS | A | 532 | 35.776 | 30.286 | 81.127 | 1.00 | 32.73 | N |
| ATOM | 1663 | N | ASP | A | 533 | 28.772 | 30.184 | 81.833 | 1.00 | 25.57 | N |
| ATOM | 1664 | CA | ASP | A | 533 | 27.760 | 29.496 | 81.044 | 1.00 | 24.91 | C |
| ATOM | 1665 | C | ASP | A | 533 | 27.406 | 30.221 | 79.746 | 1.00 | 24.32 | C |
| ATOM | 1666 | O | ASP | A | 533 | 27.838 | 31.351 | 79.507 | 1.00 | 24.30 | O |
| ATOM | 1667 | CB | ASP | A | 533 | 26.496 | 29.280 | 81.885 | 1.00 | 24.79 | C |
| ATOM | 1668 | CG | ASP | A | 533 | 25.710 | 28.057 | 81.455 | 1.00 | 25.30 | C |
| ATOM | 1669 | OD1 | ASP | A | 533 | 26.007 | 27.478 | 80.388 | 1.00 | 25.65 | O |
| ATOM | 1670 | OD2 | ASP | A | 533 | 24.783 | 27.666 | 82.186 | 1.00 | 26.36 | O |
| ATOM | 1671 | N | SER | A | 534 | 26.638 | 29.529 | 78.909 | 1.00 | 23.67 | N |
| ATOM | 1672 | CA | SER | A | 534 | 25.991 | 30.087 | 77.726 | 1.00 | 22.96 | C |
| ATOM | 1673 | C | SER | A | 534 | 24.668 | 30.735 | 78.150 | 1.00 | 22.41 | C |
| ATOM | 1674 | O | SER | A | 534 | 23.948 | 30.196 | 78.995 | 1.00 | 22.29 | O |
| ATOM | 1675 | CB | SER | A | 534 | 25.759 | 28.958 | 76.701 | 1.00 | 22.99 | C |
| ATOM | 1676 | OG | SER | A | 534 | 24.743 | 29.262 | 75.758 | 1.00 | 22.76 | O |
| ATOM | 1677 | N | TYR | A | 535 | 24.360 | 31.897 | 77.580 | 1.00 | 21.82 | N |
| ATOM | 1678 | CA | TYR | A | 535 | 23.136 | 32.629 | 77.925 | 1.00 | 21.52 | C |
| ATOM | 1679 | C | TYR | A | 535 | 22.393 | 33.117 | 76.685 | 1.00 | 21.46 | C |
| ATOM | 1680 | O | TYR | A | 535 | 22.943 | 33.138 | 75.579 | 1.00 | 21.40 | O |
| ATOM | 1681 | CB | TYR | A | 535 | 23.445 | 33.825 | 78.830 | 1.00 | 21.12 | C |
| ATOM | 1682 | CG | TYR | A | 535 | 23.998 | 33.463 | 80.188 | 1.00 | 21.17 | C |
| ATOM | 1683 | CD1 | TYR | A | 535 | 23.161 | 33.360 | 81.298 | 1.00 | 20.69 | C |
| ATOM | 1684 | CD2 | TYR | A | 535 | 25.367 | 33.245 | 80.372 | 1.00 | 20.90 | C |
| ATOM | 1685 | CE1 | TYR | A | 535 | 23.667 | 33.037 | 82.556 | 1.00 | 20.50 | C |
| ATOM | 1686 | CE2 | TYR | A | 535 | 25.881 | 32.917 | 81.627 | 1.00 | 21.33 | C |
| ATOM | 1687 | CZ | TYR | A | 535 | 25.024 | 32.819 | 82.712 | 1.00 | 20.77 | C |
| ATOM | 1688 | OH | TYR | A | 535 | 25.532 | 32.493 | 83.949 | 1.00 | 21.04 | O |
| ATOM | 1689 | N | LEU | A | 536 | 21.145 | 33.527 | 76.884 | 1.00 | 21.26 | N |
| ATOM | 1690 | CA | LEU | A | 536 | 20.302 | 33.978 | 75.790 | 1.00 | 21.36 | C |
| ATOM | 1691 | C | LEU | A | 536 | 19.622 | 35.298 | 76.137 | 1.00 | 21.45 | C |
| ATOM | 1692 | O | LEU | A | 536 | 18.857 | 35.378 | 77.098 | 1.00 | 21.67 | O |
| ATOM | 1693 | CB | LEU | A | 536 | 19.266 | 32.907 | 75.437 | 1.00 | 21.00 | C |
| ATOM | 1694 | CG | LEU | A | 536 | 18.683 | 33.018 | 74.034 | 1.00 | 21.29 | C |
| ATOM | 1695 | CD1 | LEU | A | 536 | 19.771 | 32.814 | 72.980 | 1.00 | 21.36 | C |
| ATOM | 1696 | CD2 | LEU | A | 536 | 17.551 | 32.027 | 73.847 | 1.00 | 20.77 | C |
| ATOM | 1697 | N | TRP | A | 537 | 19.910 | 36.321 | 75.340 | 1.00 | 21.34 | N |
| ATOM | 1698 | CA | TRP | A | 537 | 19.452 | 37.674 | 75.596 | 1.00 | 21.48 | C |
| ATOM | 1699 | C | TRP | A | 537 | 18.544 | 38.178 | 74.488 | 1.00 | 21.41 | C |
| ATOM | 1700 | O | TRP | A | 537 | 18.598 | 37.690 | 73.364 | 1.00 | 21.48 | O |
| ATOM | 1701 | CB | TRP | A | 537 | 20.656 | 38.608 | 75.740 | 1.00 | 21.74 | C |
| ATOM | 1702 | CG | TRP | A | 537 | 21.345 | 38.546 | 77.082 | 1.00 | 21.86 | C |
| ATOM | 1703 | CD1 | TRP | A | 537 | 21.142 | 37.627 | 78.072 | 1.00 | 21.36 | C |
| ATOM | 1704 | CD2 | TRP | A | 537 | 22.372 | 39.429 | 77.557 | 1.00 | 21.92 | C |
| ATOM | 1705 | NE1 | TRP | A | 537 | 21.964 | 37.895 | 79.138 | 1.00 | 22.21 | N |
| ATOM | 1706 | CE2 | TRP | A | 537 | 22.729 | 38.995 | 78.851 | 1.00 | 21.85 | C |
| ATOM | 1707 | CE3 | TRP | A | 537 | 23.019 | 40.550 | 77.016 | 1.00 | 21.71 | C |
| ATOM | 1708 | CZ2 | TRP | A | 537 | 23.707 | 39.641 | 79.618 | 1.00 | 21.83 | C |
| ATOM | 1709 | CZ3 | TRP | A | 537 | 23.990 | 41.196 | 77.779 | 1.00 | 21.94 | C |
| ATOM | 1710 | CH2 | TRP | A | 537 | 24.324 | 40.738 | 79.068 | 1.00 | 21.57 | C |
| ATOM | 1711 | N | MET | A | 538 | 17.717 | 39.165 | 74.814 | 1.00 | 21.49 | N |
| ATOM | 1712 | CA | MET | A | 538 | 16.820 | 39.768 | 73.835 | 1.00 | 21.88 | C |
| ATOM | 1713 | C | MET | A | 538 | 16.675 | 41.271 | 74.057 | 1.00 | 21.87 | C |
| ATOM | 1714 | O | MET | A | 538 | 16.595 | 41.734 | 75.192 | 1.00 | 22.10 | O |
| ATOM | 1715 | CB | MET | A | 538 | 15.453 | 39.072 | 73.878 | 1.00 | 21.82 | C |
| ATOM | 1716 | CG | MET | A | 538 | 14.382 | 39.685 | 72.992 | 1.00 | 22.02 | C |
| ATOM | 1717 | SD | MET | A | 538 | 13.535 | 41.086 | 73.760 | 1.00 | 23.50 | S |
| ATOM | 1718 | CE | MET | A | 538 | 12.470 | 40.222 | 74.922 | 1.00 | 21.20 | C |
| ATOM | 1719 | N | SER | A | 539 | 16.643 | 42.027 | 72.966 | 1.00 | 22.06 | N |
| ATOM | 1720 | CA | SER | A | 539 | 16.346 | 43.460 | 73.019 | 1.00 | 22.23 | C |
| ATOM | 1721 | C | SER | A | 539 | 15.248 | 43.790 | 72.005 | 1.00 | 22.62 | C |
| ATOM | 1722 | O | SER | A | 539 | 15.156 | 43.145 | 70.955 | 1.00 | 22.72 | O |
| ATOM | 1723 | CB | SER | A | 539 | 17.602 | 44.289 | 72.746 | 1.00 | 22.15 | C |
| ATOM | 1724 | OG | SER | A | 539 | 18.669 | 43.932 | 73.617 | 1.00 | 21.56 | O |
| ATOM | 1725 | N | TYR | A | 540 | 14.416 | 44.782 | 72.322 | 1.00 | 22.96 | N |
| ATOM | 1726 | CA | TYR | A | 540 | 13.294 | 45.142 | 71.453 | 1.00 | 23.35 | C |
| ATOM | 1727 | C | TYR | A | 540 | 13.260 | 46.609 | 71.035 | 1.00 | 23.56 | C |
| ATOM | 1728 | O | TYR | A | 540 | 13.809 | 47.477 | 71.719 | 1.00 | 23.63 | O |
| ATOM | 1729 | CB | TYR | A | 540 | 11.951 | 44.732 | 72.079 | 1.00 | 23.36 | C |
| ATOM | 1730 | CG | TYR | A | 540 | 11.627 | 45.406 | 73.399 | 1.00 | 23.63 | C |
| ATOM | 1731 | CD1 | TYR | A | 540 | 11.150 | 46.719 | 73.435 | 1.00 | 23.15 | C |
| ATOM | 1732 | CD2 | TYR | A | 540 | 11.775 | 44.721 | 74.611 | 1.00 | 23.64 | C |
| ATOM | 1733 | CE1 | TYR | A | 540 | 10.846 | 47.341 | 74.639 | 1.00 | 23.50 | C |
| ATOM | 1734 | CE2 | TYR | A | 540 | 11.470 | 45.333 | 75.824 | 1.00 | 24.11 | C |
| ATOM | 1735 | CZ | TYR | A | 540 | 11.006 | 46.648 | 75.828 | 1.00 | 24.09 | C |
| ATOM | 1736 | OH | TYR | A | 540 | 10.706 | 47.273 | 77.014 | 1.00 | 23.93 | O |
| ATOM | 1737 | N | SER | A | 541 | 12.592 | 46.868 | 69.914 | 1.00 | 23.89 | N |
| ATOM | 1738 | CA | SER | A | 541 | 12.409 | 48.217 | 69.401 | 1.00 | 24.33 | C |
| ATOM | 1739 | C | SER | A | 541 | 10.948 | 48.509 | 69.068 | 1.00 | 24.73 | C |
| ATOM | 1740 | O | SER | A | 541 | 10.296 | 47.741 | 68.355 | 1.00 | 24.68 | O |
| ATOM | 1741 | CB | SER | A | 541 | 13.264 | 48.422 | 68.158 | 1.00 | 24.45 | C |
| ATOM | 1742 | OG | SER | A | 541 | 13.055 | 49.712 | 67.622 | 1.00 | 25.02 | O |
| ATOM | 1743 | N | ASP | A | 542 | 10.443 | 49.627 | 69.581 | 1.00 | 25.11 | N |
| ATOM | 1744 | CA | ASP | A | 542 | 9.080 | 50.055 | 69.280 | 1.00 | 25.40 | C |
| ATOM | 1745 | C | ASP | A | 542 | 9.031 | 51.241 | 68.321 | 1.00 | 25.74 | C |
| ATOM | 1746 | O | ASP | A | 542 | 7.955 | 51.772 | 68.035 | 1.00 | 25.86 | O |
| ATOM | 1747 | CB | ASP | A | 542 | 8.313 | 50.365 | 70.570 | 1.00 | 25.32 | C |
| ATOM | 1748 | CG | ASP | A | 542 | 8.018 | 49.117 | 71.393 | 1.00 | 25.35 | C |
| ATOM | 1749 | OD1 | ASP | A | 542 | 7.687 | 48.063 | 70.812 | 1.00 | 25.13 | O |
| ATOM | 1750 | OD2 | ASP | A | 542 | 8.105 | 49.194 | 72.633 | 1.00 | 26.19 | O |
| ATOM | 1751 | N | ASP | A | 543 | 10.192 | 51.662 | 67.824 | 1.00 | 26.17 | N |
| ATOM | 1752 | CA | ASP | A | 543 | 10.230 | 52.741 | 66.836 | 1.00 | 26.68 | C |
| ATOM | 1753 | C | ASP | A | 543 | 10.954 | 52.329 | 65.552 | 1.00 | 26.94 | C |
| ATOM | 1754 | O | ASP | A | 543 | 11.822 | 53.050 | 65.043 | 1.00 | 27.13 | O |
| ATOM | 1755 | CB | ASP | A | 543 | 10.805 | 54.035 | 67.434 | 1.00 | 26.51 | C |
| ATOM | 1756 | CG | ASP | A | 543 | 12.203 | 53.860 | 68.007 | 1.00 | 27.10 | C |
| ATOM | 1757 | OD1 | ASP | A | 543 | 12.841 | 52.797 | 67.811 | 1.00 | 26.77 | O |
| ATOM | 1758 | OD2 | ASP | A | 543 | 12.669 | 54.811 | 68.662 | 1.00 | 28.02 | O |
| ATOM | 1759 | N | ASP | A | 544 | 10.587 | 51.157 | 65.043 | 1.00 | 27.29 | N |
| ATOM | 1760 | CA | ASP | A | 544 | 11.036 | 50.684 | 63.730 | 1.00 | 27.81 | C |
| ATOM | 1761 | C | ASP | A | 544 | 12.555 | 50.480 | 63.631 | 1.00 | 27.84 | C |
| ATOM | 1762 | O | ASP | A | 544 | 13.132 | 50.585 | 62.550 | 1.00 | 27.85 | O |
| ATOM | 1763 | CB | ASP | A | 544 | 10.517 | 51.617 | 62.614 | 1.00 | 27.86 | C |
| ATOM | 1764 | CG | ASP | A | 544 | 10.568 | 50.977 | 61.241 | 1.00 | 28.21 | C |
| ATOM | 1765 | OD1 | ASP | A | 544 | 10.112 | 49.820 | 61.093 | 1.00 | 29.09 | O |
| ATOM | 1766 | OD2 | ASP | A | 544 | 11.060 | 51.640 | 60.306 | 1.00 | 28.32 | O |
| ATOM | 1767 | N | GLY | A | 545 | 13.190 | 50.185 | 64.764 | 1.00 | 28.04 | N |
| ATOM | 1768 | CA | GLY | A | 545 | 14.606 | 49.814 | 64.787 | 1.00 | 28.17 | C |
| ATOM | 1769 | C | GLY | A | 545 | 15.589 | 50.926 | 65.114 | 1.00 | 28.42 | C |
| ATOM | 1770 | O | GLY | A | 545 | 16.804 | 50.706 | 65.116 | 1.00 | 28.60 | O |
| ATOM | 1771 | N | LYS | A | 546 | 15.068 | 52.115 | 65.400 | 1.00 | 28.61 | N |
| ATOM | 1772 | CA | LYS | A | 546 | 15.896 | 53.289 | 65.680 | 1.00 | 28.73 | C |
| ATOM | 1773 | C | LYS | A | 546 | 16.485 | 53.274 | 67.095 | 1.00 | 28.87 | C |
| ATOM | 1774 | O | LYS | A | 546 | 17.669 | 53.558 | 67.275 | 1.00 | 29.14 | O |
| ATOM | 1775 | CB | LYS | A | 546 | 15.096 | 54.570 | 65.428 | 1.00 | 28.86 | C |
| ATOM | 1776 | CG | LYS | A | 546 | 14.695 | 54.762 | 63.965 | 1.00 | 29.03 | C |
| ATOM | 1777 | CD | LYS | A | 546 | 13.699 | 55.894 | 63.797 | 1.00 | 30.05 | C |
| ATOM | 1778 | CE | LYS | A | 546 | 13.745 | 56.454 | 62.383 | 1.00 | 30.86 | C |
| ATOM | 1779 | NZ | LYS | A | 546 | 12.661 | 57.440 | 62.127 | 1.00 | 31.61 | N |
| ATOM | 1780 | N | THR | A | 547 | 15.661 | 52.942 | 68.090 | 1.00 | 28.74 | N |
| ATOM | 1781 | CA | THR | A | 547 | 16.126 | 52.807 | 69.475 | 1.00 | 28.54 | C |
| ATOM | 1782 | C | THR | A | 547 | 15.737 | 51.441 | 70.040 | 1.00 | 28.43 | C |
| ATOM | 1783 | O | THR | A | 547 | 14.698 | 50.882 | 69.683 | 1.00 | 28.29 | O |
| ATOM | 1784 | CB | THR | A | 547 | 15.572 | 53.922 | 70.406 | 1.00 | 28.80 | C |
| ATOM | 1785 | OG1 | THR | A | 547 | 14.136 | 53.843 | 70.464 | 1.00 | 28.91 | O |
| ATOM | 1786 | CG2 | THR | A | 547 | 16.009 | 55.314 | 69.927 | 1.00 | 28.22 | C |
| ATOM | 1787 | N | TRP | A | 548 | 16.573 | 50.922 | 70.933 | 1.00 | 28.17 | N |
| ATOM | 1788 | CA | TRP | A | 548 | 16.426 | 49.566 | 71.437 | 1.00 | 27.97 | C |
| ATOM | 1789 | C | TRP | A | 548 | 16.389 | 49.544 | 72.949 | 1.00 | 27.97 | C |
| ATOM | 1790 | O | TRP | A | 548 | 16.981 | 50.404 | 73.606 | 1.00 | 27.87 | O |
| ATOM | 1791 | CB | TRP | A | 548 | 17.576 | 48.692 | 70.933 | 1.00 | 28.04 | C |
| ATOM | 1792 | CG | TRP | A | 548 | 17.528 | 48.488 | 69.463 | 1.00 | 28.17 | C |
| ATOM | 1793 | CD1 | TRP | A | 548 | 17.988 | 49.343 | 68.502 | 1.00 | 28.22 | C |
| ATOM | 1794 | CD2 | TRP | A | 548 | 16.964 | 47.367 | 68.771 | 1.00 | 28.20 | C |
| ATOM | 1795 | NE1 | TRP | A | 548 | 17.753 | 48.818 | 67.252 | 1.00 | 29.06 | N |
| ATOM | 1796 | CE2 | TRP | A | 548 | 17.127 | 47.606 | 67.387 | 1.00 | 28.50 | C |
| ATOM | 1797 | CE3 | TRP | A | 548 | 16.342 | 46.180 | 69.184 | 1.00 | 28.65 | C |
| ATOM | 1798 | CZ2 | TRP | A | 548 | 16.684 | 46.702 | 66.410 | 1.00 | 28.01 | C |
| ATOM | 1799 | CZ3 | TRP | A | 548 | 15.906 | 45.275 | 68.208 | 1.00 | 28.33 | C |
| ATOM | 1800 | CH2 | TRP | A | 548 | 16.082 | 45.545 | 66.841 | 1.00 | 27.91 | C |
| ATOM | 1801 | N | SER | A | 549 | 15.695 | 48.550 | 73.495 | 1.00 | 27.79 | N |
| ATOM | 1802 | CA | SER | A | 549 | 15.620 | 48.372 | 74.936 | 1.00 | 27.72 | C |
| ATOM | 1803 | C | SER | A | 549 | 16.931 | 47.797 | 75.461 | 1.00 | 27.56 | C |
| ATOM | 1804 | O | SER | A | 549 | 17.749 | 47.284 | 74.690 | 1.00 | 27.59 | O |
| ATOM | 1805 | CB | SER | A | 549 | 14.454 | 47.448 | 75.297 | 1.00 | 27.62 | C |
| ATOM | 1806 | OG | SER | A | 549 | 14.645 | 46.150 | 74.752 | 1.00 | 28.57 | O |
| ATOM | 1807 | N | ALA | A | 550 | 17.122 | 47.905 | 76.773 | 1.00 | 27.43 | N |
| ATOM | 1808 | CA | ALA | A | 550 | 18.213 | 47.235 | 77.476 | 1.00 | 27.32 | C |
| ATOM | 1809 | C | ALA | A | 550 | 17.996 | 45.725 | 77.398 | 1.00 | 27.25 | C |
| ATOM | 1810 | O | ALA | A | 550 | 16.849 | 45.269 | 77.391 | 1.00 | 27.69 | O |
| ATOM | 1811 | CB | ALA | A | 550 | 18.257 | 47.692 | 78.924 | 1.00 | 27.21 | C |
| ATOM | 1812 | N | PRO | A | 551 | 19.086 | 44.942 | 77.339 | 1.00 | 26.94 | N |
| ATOM | 1813 | CA | PRO | A | 551 | 18.968 | 43.493 | 77.138 | 1.00 | 26.66 | C |
| ATOM | 1814 | C | PRO | A | 551 | 18.143 | 42.784 | 78.208 | 1.00 | 26.35 | C |
| ATOM | 1815 | O | PRO | A | 551 | 18.410 | 42.939 | 79.401 | 1.00 | 26.41 | O |
| ATOM | 1816 | CB | PRO | A | 551 | 20.425 | 43.011 | 77.205 | 1.00 | 26.71 | C |
| ATOM | 1817 | CG | PRO | A | 551 | 21.241 | 44.207 | 76.874 | 1.00 | 26.71 | C |
| ATOM | 1818 | CD | PRO | A | 551 | 20.493 | 45.366 | 77.454 | 1.00 | 26.95 | C |
| ATOM | 1819 | N | GLN | A | 552 | 17.139 | 42.025 | 77.781 | 1.00 | 26.07 | N |
| ATOM | 1820 | CA | GLN | A | 552 | 16.425 | 41.136 | 78.689 | 1.00 | 25.63 | C |
| ATOM | 1821 | C | GLN | A | 552 | 17.076 | 39.777 | 78.663 | 1.00 | 25.18 | C |
| ATOM | 1822 | O | GLN | A | 552 | 17.300 | 39.206 | 77.589 | 1.00 | 24.92 | O |
| ATOM | 1823 | CB | GLN | A | 552 | 14.949 | 41.001 | 78.320 | 1.00 | 26.09 | C |
| ATOM | 1824 | CG | GLN | A | 552 | 14.003 | 41.803 | 79.196 | 1.00 | 27.73 | C |
| ATOM | 1825 | CD | GLN | A | 552 | 14.043 | 43.280 | 78.882 | 1.00 | 30.53 | C |
| ATOM | 1826 | OE1 | GLN | A | 552 | 14.367 | 43.678 | 77.760 | 1.00 | 32.40 | O |
| ATOM | 1827 | NE2 | GLN | A | 552 | 13.716 | 44.105 | 79.869 | 1.00 | 31.19 | N |
| ATOM | 1828 | N | ASP | A | 553 | 17.387 | 39.273 | 79.854 | 1.00 | 24.65 | N |
| ATOM | 1829 | CA | ASP | A | 553 | 17.905 | 37.931 | 80.018 | 1.00 | 24.03 | C |
| ATOM | 1830 | C | ASP | A | 553 | 16.726 | 36.974 | 80.008 | 1.00 | 23.59 | C |
| ATOM | 1831 | O | ASP | A | 553 | 15.962 | 36.915 | 80.968 | 1.00 | 23.64 | O |
| ATOM | 1832 | CB | ASP | A | 553 | 18.660 | 37.818 | 81.338 | 1.00 | 23.99 | C |
| ATOM | 1833 | CG | ASP | A | 553 | 19.368 | 36.485 | 81.500 | 1.00 | 24.33 | C |
| ATOM | 1834 | OD1 | ASP | A | 553 | 19.552 | 35.751 | 80.500 | 1.00 | 23.49 | O |
| ATOM | 1835 | OD2 | ASP | A | 553 | 19.757 | 36.176 | 82.642 | 1.00 | 25.83 | O |
| ATOM | 1836 | N | ILE | A | 554 | 16.578 | 36.228 | 78.920 | 1.00 | 23.09 | N |
| ATOM | 1837 | CA | ILE | A | 554 | 15.449 | 35.302 | 78.771 | 1.00 | 22.53 | C |
| ATOM | 1838 | C | ILE | A | 554 | 15.846 | 33.855 | 79.033 | 1.00 | 22.09 | C |
| ATOM | 1839 | O | ILE | A | 554 | 15.003 | 32.958 | 78.990 | 1.00 | 22.08 | O |
| ATOM | 1840 | CB | ILE | A | 554 | 14.758 | 35.439 | 77.384 | 1.00 | 22.65 | C |
| ATOM | 1841 | CG1 | ILE | A | 554 | 15.726 | 35.098 | 76.243 | 1.00 | 22.34 | C |
| ATOM | 1842 | CG2 | ILE | A | 554 | 14.169 | 36.847 | 77.225 | 1.00 | 22.31 | C |
| ATOM | 1843 | CD1 | ILE | A | 554 | 15.041 | 34.674 | 74.949 | 1.00 | 23.41 | C |
| ATOM | 1844 | N | THR | A | 555 | 17.131 | 33.654 | 79.324 | 1.00 | 21.80 | N |
| ATOM | 1845 | CA | THR | A | 555 | 17.732 | 32.327 | 79.559 | 1.00 | 21.59 | C |
| ATOM | 1846 | C | THR | A | 555 | 16.886 | 31.367 | 80.411 | 1.00 | 21.45 | C |
| ATOM | 1847 | O | THR | A | 555 | 16.579 | 30.266 | 79.949 | 1.00 | 21.27 | O |
| ATOM | 1848 | CB | THR | A | 555 | 19.168 | 32.445 | 80.164 | 1.00 | 21.35 | C |
| ATOM | 1849 | OG1 | THR | A | 555 | 19.926 | 33.415 | 79.432 | 1.00 | 21.97 | O |
| ATOM | 1850 | CG2 | THR | A | 555 | 19.903 | 31.106 | 80.142 | 1.00 | 21.14 | C |
| ATOM | 1851 | N | PRO | A | 556 | 16.498 | 31.780 | 81.645 | 1.00 | 21.63 | N |
| ATOM | 1852 | CA | PRO | A | 556 | 15.827 | 30.821 | 82.541 | 1.00 | 21.64 | C |
| ATOM | 1853 | C | PRO | A | 556 | 14.473 | 30.332 | 82.036 | 1.00 | 21.70 | C |
| ATOM | 1854 | O | PRO | A | 556 | 13.992 | 29.295 | 82.485 | 1.00 | 21.75 | O |
| ATOM | 1855 | CB | PRO | A | 556 | 15.670 | 31.601 | 83.857 | 1.00 | 21.58 | C |
| ATOM | 1856 | CG | PRO | A | 556 | 15.732 | 33.039 | 83.468 | 1.00 | 21.67 | C |
| ATOM | 1857 | CD | PRO | A | 556 | 16.636 | 33.114 | 82.274 | 1.00 | 21.44 | C |
| ATOM | 1858 | N | MET | A | 557 | 13.870 | 31.050 | 81.094 | 1.00 | 22.12 | N |
| ATOM | 1859 | CA | MET | A | 557 | 12.574 | 30.635 | 80.541 | 1.00 | 22.38 | C |
| ATOM | 1860 | C | MET | A | 557 | 12.692 | 29.412 | 79.627 | 1.00 | 22.10 | C |
| ATOM | 1861 | O | MET | A | 557 | 11.742 | 28.641 | 79.483 | 1.00 | 22.03 | O |
| ATOM | 1862 | CB | MET | A | 557 | 11.905 | 31.780 | 79.777 | 1.00 | 22.16 | C |
| ATOM | 1863 | CG | MET | A | 557 | 11.863 | 33.109 | 80.514 | 1.00 | 22.83 | C |
| ATOM | 1864 | SD | MET | A | 557 | 10.670 | 34.245 | 79.779 | 1.00 | 23.61 | S |
| ATOM | 1865 | CE | MET | A | 557 | 11.287 | 35.833 | 80.332 | 1.00 | 23.16 | C |
| ATOM | 1866 | N | VAL | A | 558 | 13.862 | 29.232 | 79.020 | 1.00 | 22.14 | N |
| ATOM | 1867 | CA | VAL | A | 558 | 14.007 | 28.288 | 77.910 | 1.00 | 22.04 | C |
| ATOM | 1868 | C | VAL | A | 558 | 15.125 | 27.259 | 78.054 | 1.00 | 22.23 | C |
| ATOM | 1869 | O | VAL | A | 558 | 15.138 | 26.256 | 77.333 | 1.00 | 21.96 | O |
| ATOM | 1870 | CB | VAL | A | 558 | 14.193 | 29.039 | 76.570 | 1.00 | 22.30 | C |
| ATOM | 1871 | CG1 | VAL | A | 558 | 12.923 | 29.813 | 76.211 | 1.00 | 21.48 | C |
| ATOM | 1872 | CG2 | VAL | A | 558 | 15.427 | 29.970 | 76.621 | 1.00 | 21.72 | C |
| ATOM | 1873 | N | LYS | A | 559 | 16.054 | 27.502 | 78.976 | 1.00 | 22.46 | N |
| ATOM | 1874 | CA | LYS | A | 559 | 17.221 | 26.632 | 79.134 | 1.00 | 22.60 | C |
| ATOM | 1875 | C | LYS | A | 559 | 17.074 | 25.593 | 80.249 | 1.00 | 22.93 | C |
| ATOM | 1876 | O | LYS | A | 559 | 16.975 | 25.951 | 81.416 | 1.00 | 23.29 | O |
| ATOM | 1877 | CB | LYS | A | 559 | 18.475 | 27.470 | 79.378 | 1.00 | 22.48 | C |
| ATOM | 1878 | CG | LYS | A | 559 | 19.737 | 26.674 | 79.211 | 1.00 | 21.49 | C |
| ATOM | 1879 | CD | LYS | A | 559 | 20.961 | 27.498 | 79.438 | 1.00 | 19.97 | C |
| ATOM | 1880 | CE | LYS | A | 559 | 22.140 | 26.744 | 78.902 | 1.00 | 19.47 | C |
| ATOM | 1881 | NZ | LYS | A | 559 | 23.324 | 26.904 | 79.758 | 1.00 | 20.96 | N |
| ATOM | 1882 | N | ALA | A | 560 | 17.100 | 24.311 | 79.882 | 1.00 | 23.25 | N |
| ATOM | 1883 | CA | ALA | A | 560 | 16.967 | 23.204 | 80.844 | 1.00 | 23.46 | C |
| ATOM | 1884 | C | ALA | A | 560 | 18.249 | 22.924 | 81.648 | 1.00 | 23.73 | C |
| ATOM | 1885 | O | ALA | A | 560 | 19.324 | 23.423 | 81.307 | 1.00 | 23.93 | O |
| ATOM | 1886 | CB | ALA | A | 560 | 16.500 | 21.940 | 80.130 | 1.00 | 23.36 | C |
| ATOM | 1887 | N | ASP | A | 561 | 18.117 | 22.115 | 82.705 | 1.00 | 24.04 | N |
| ATOM | 1888 | CA | ASP | A | 561 | 19.216 | 21.780 | 83.624 | 1.00 | 24.19 | C |
| ATOM | 1889 | C | ASP | A | 561 | 20.381 | 21.078 | 82.937 | 1.00 | 23.90 | C |
| ATOM | 1890 | O | ASP | A | 561 | 21.529 | 21.232 | 83.353 | 1.00 | 24.25 | O |
| ATOM | 1891 | CB | ASP | A | 561 | 18.735 | 20.863 | 84.758 | 1.00 | 24.46 | C |
| ATOM | 1892 | CG | ASP | A | 561 | 17.573 | 21.437 | 85.545 | 1.00 | 26.44 | C |
| ATOM | 1893 | OD1 | ASP | A | 561 | 17.474 | 22.681 | 85.673 | 1.00 | 29.36 | O |
| ATOM | 1894 | OD2 | ASP | A | 561 | 16.754 | 20.631 | 86.052 | 1.00 | 27.87 | O |
| ATOM | 1895 | N | TRP | A | 562 | 20.085 | 20.281 | 81.913 | 1.00 | 23.33 | N |
| ATOM | 1896 | CA | TRP | A | 562 | 21.116 | 19.464 | 81.264 | 1.00 | 22.83 | C |
| ATOM | 1897 | C | TRP | A | 562 | 21.865 | 20.220 | 80.174 | 1.00 | 22.36 | C |
| ATOM | 1898 | O | TRP | A | 562 | 22.978 | 19.831 | 79.801 | 1.00 | 22.06 | O |
| ATOM | 1899 | CB | TRP | A | 562 | 20.513 | 18.188 | 80.680 | 1.00 | 22.60 | C |
| ATOM | 1900 | CG | TRP | A | 562 | 19.403 | 18.437 | 79.698 | 1.00 | 22.81 | C |
| ATOM | 1901 | CD1 | TRP | A | 562 | 18.067 | 18.453 | 79.968 | 1.00 | 22.92 | C |
| ATOM | 1902 | CD2 | TRP | A | 562 | 19.532 | 18.711 | 78.292 | 1.00 | 22.95 | C |
| ATOM | 1903 | NE1 | TRP | A | 562 | 17.354 | 18.709 | 78.823 | 1.00 | 22.94 | N |
| ATOM | 1904 | CE2 | TRP | A | 562 | 18.225 | 18.867 | 77.778 | 1.00 | 22.62 | C |
| ATOM | 1905 | CE3 | TRP | A | 562 | 20.623 | 18.822 | 77.415 | 1.00 | 22.37 | C |
| ATOM | 1906 | CZ2 | TRP | A | 562 | 17.974 | 19.133 | 76.428 | 1.00 | 22.30 | C |
| ATOM | 1907 | CZ3 | TRP | A | 562 | 20.373 | 19.086 | 76.069 | 1.00 | 22.53 | C |
| ATOM | 1908 | CH2 | TRP | A | 562 | 19.057 | 19.241 | 75.591 | 1.00 | 22.74 | C |
| ATOM | 1909 | N | MET | A | 563 | 21.238 | 21.285 | 79.668 | 1.00 | 21.57 | N |
| ATOM | 1910 | CA | MET | A | 563 | 21.751 | 22.029 | 78.518 | 1.00 | 20.97 | C |
| ATOM | 1911 | C | MET | A | 563 | 23.093 | 22.673 | 78.838 | 1.00 | 20.95 | C |
| ATOM | 1912 | O | MET | A | 563 | 23.258 | 23.317 | 79.884 | 1.00 | 20.81 | O |
| ATOM | 1913 | CB | MET | A | 563 | 20.730 | 23.073 | 78.025 | 1.00 | 20.70 | C |
| ATOM | 1914 | CG | MET | A | 563 | 19.489 | 22.478 | 77.311 | 1.00 | 20.52 | C |
| ATOM | 1915 | SD | MET | A | 563 | 18.203 | 23.686 | 76.860 | 1.00 | 20.70 | S |
| ATOM | 1916 | CE | MET | A | 563 | 16.977 | 22.648 | 76.072 | 1.00 | 20.16 | C |
| ATOM | 1917 | N | LYS | A | 564 | 24.063 | 22.457 | 77.952 | 1.00 | 20.57 | N |
| ATOM | 1918 | CA | LYS | A | 564 | 25.328 | 23.162 | 78.039 | 1.00 | 20.24 | C |
| ATOM | 1919 | C | LYS | A | 564 | 25.184 | 24.452 | 77.238 | 1.00 | 19.66 | C |
| ATOM | 1920 | O | LYS | A | 564 | 24.902 | 25.511 | 77.802 | 1.00 | 19.66 | O |
| ATOM | 1921 | CB | LYS | A | 564 | 26.485 | 22.299 | 77.526 | 1.00 | 20.36 | C |
| ATOM | 1922 | CG | LYS | A | 564 | 27.862 | 22.832 | 77.912 | 1.00 | 20.67 | C |
| ATOM | 1923 | CD | LYS | A | 564 | 28.993 | 21.905 | 77.470 | 1.00 | 20.79 | C |
| ATOM | 1924 | CE | LYS | A | 564 | 30.272 | 22.261 | 78.221 | 1.00 | 22.25 | C |
| ATOM | 1925 | NZ | LYS | A | 564 | 31.449 | 21.492 | 77.743 | 1.00 | 24.17 | N |
| ATOM | 1926 | N | PHE | A | 565 | 25.362 | 24.353 | 75.926 | 1.00 | 18.86 | N |
| ATOM | 1927 | CA | PHE | A | 565 | 25.126 | 25.476 | 75.040 | 1.00 | 18.48 | C |
| ATOM | 1928 | C | PHE | A | 565 | 23.655 | 25.578 | 74.645 | 1.00 | 18.46 | C |
| ATOM | 1929 | O | PHE | A | 565 | 23.008 | 24.572 | 74.354 | 1.00 | 18.37 | O |
| ATOM | 1930 | CB | PHE | A | 565 | 25.992 | 25.364 | 73.780 | 1.00 | 18.08 | C |
| ATOM | 1931 | CG | PHE | A | 565 | 25.680 | 26.402 | 72.733 | 1.00 | 17.18 | C |
| ATOM | 1932 | CD1 | PHE | A | 565 | 26.076 | 27.723 | 72.901 | 1.00 | 16.42 | C |
| ATOM | 1933 | CD2 | PHE | A | 565 | 24.985 | 26.056 | 71.581 | 1.00 | 16.53 | C |
| ATOM | 1934 | CE1 | PHE | A | 565 | 25.789 | 28.686 | 71.932 | 1.00 | 16.62 | C |
| ATOM | 1935 | CE2 | PHE | A | 565 | 24.688 | 27.014 | 70.606 | 1.00 | 17.15 | C |
| ATOM | 1936 | CZ | PHE | A | 565 | 25.098 | 28.330 | 70.780 | 1.00 | 16.71 | C |
| ATOM | 1937 | N | LEU | A | 566 | 23.140 | 26.804 | 74.640 | 1.00 | 18.37 | N |
| ATOM | 1938 | CA | LEU | A | 566 | 21.879 | 27.119 | 73.977 | 1.00 | 18.25 | C |
| ATOM | 1939 | C | LEU | A | 566 | 22.048 | 28.453 | 73.272 | 1.00 | 18.28 | C |
| ATOM | 1940 | O | LEU | A | 566 | 22.221 | 29.489 | 73.924 | 1.00 | 18.49 | O |
| ATOM | 1941 | CB | LEU | A | 566 | 20.715 | 27.182 | 74.973 | 1.00 | 18.09 | C |
| ATOM | 1942 | CG | LEU | A | 566 | 19.424 | 27.841 | 74.459 | 1.00 | 17.66 | C |
| ATOM | 1943 | CD1 | LEU | A | 566 | 18.679 | 26.951 | 73.469 | 1.00 | 18.03 | C |
| ATOM | 1944 | CD2 | LEU | A | 566 | 18.528 | 28.215 | 75.614 | 1.00 | 17.22 | C |
| ATOM | 1945 | N | GLY | A | 567 | 22.004 | 28.420 | 71.944 | 1.00 | 18.21 | N |
| ATOM | 1946 | CA | GLY | A | 567 | 22.167 | 29.630 | 71.139 | 1.00 | 18.37 | C |
| ATOM | 1947 | C | GLY | A | 567 | 21.253 | 29.676 | 69.933 | 1.00 | 18.34 | C |
| ATOM | 1948 | O | GLY | A | 567 | 20.693 | 28.661 | 69.530 | 1.00 | 18.16 | O |
| ATOM | 1949 | N | VAL | A | 568 | 21.108 | 30.864 | 69.358 | 1.00 | 18.41 | N |
| ATOM | 1950 | CA | VAL | A | 568 | 20.221 | 31.063 | 68.222 | 1.00 | 18.52 | C |
| ATOM | 1951 | C | VAL | A | 568 | 20.773 | 30.433 | 66.947 | 1.00 | 18.88 | C |
| ATOM | 1952 | O | VAL | A | 568 | 21.996 | 30.273 | 66.790 | 1.00 | 18.51 | O |
| ATOM | 1953 | CB | VAL | A | 568 | 19.935 | 32.568 | 67.954 | 1.00 | 18.65 | C |
| ATOM | 1954 | CG1 | VAL | A | 568 | 19.030 | 33.175 | 69.041 | 1.00 | 18.14 | C |
| ATOM | 1955 | CG2 | VAL | A | 568 | 21.222 | 33.354 | 67.819 | 1.00 | 18.61 | C |
| ATOM | 1956 | N | GLY | A | 569 | 19.852 | 30.050 | 66.062 | 1.00 | 19.03 | N |
| ATOM | 1957 | CA | GLY | A | 569 | 20.164 | 29.767 | 64.667 | 1.00 | 19.11 | C |
| ATOM | 1958 | C | GLY | A | 569 | 19.998 | 31.082 | 63.929 | 1.00 | 19.34 | C |
| ATOM | 1959 | O | GLY | A | 569 | 18.893 | 31.419 | 63.495 | 1.00 | 19.18 | O |
| ATOM | 1960 | N | PRO | A | 570 | 21.100 | 31.834 | 63.774 | 1.00 | 19.64 | N |
| ATOM | 1961 | CA | PRO | A | 570 | 21.038 | 33.244 | 63.381 | 1.00 | 19.73 | C |
| ATOM | 1962 | C | PRO | A | 570 | 20.394 | 33.470 | 62.022 | 1.00 | 19.99 | C |
| ATOM | 1963 | O | PRO | A | 570 | 20.418 | 32.585 | 61.157 | 1.00 | 19.62 | O |
| ATOM | 1964 | CB | PRO | A | 570 | 22.507 | 33.659 | 63.337 | 1.00 | 19.84 | C |
| ATOM | 1965 | CG | PRO | A | 570 | 23.258 | 32.379 | 63.134 | 1.00 | 19.84 | C |
| ATOM | 1966 | CD | PRO | A | 570 | 22.491 | 31.372 | 63.924 | 1.00 | 19.69 | C |
| ATOM | 1967 | N | GLY | A | 571 | 19.832 | 34.662 | 61.851 | 1.00 | 20.05 | N |
| ATOM | 1968 | CA | GLY | A | 571 | 19.096 | 35.010 | 60.654 | 1.00 | 20.46 | C |
| ATOM | 1969 | C | GLY | A | 571 | 17.796 | 35.670 | 61.046 | 1.00 | 20.88 | C |
| ATOM | 1970 | O | GLY | A | 571 | 17.756 | 36.425 | 62.021 | 1.00 | 20.87 | O |
| ATOM | 1971 | N | THR | A | 572 | 16.732 | 35.373 | 60.298 | 1.00 | 21.17 | N |
| ATOM | 1972 | CA | THR | A | 572 | 15.439 | 36.020 | 60.496 | 1.00 | 21.59 | C |
| ATOM | 1973 | C | THR | A | 572 | 14.352 | 35.017 | 60.868 | 1.00 | 21.72 | C |
| ATOM | 1974 | O | THR | A | 572 | 14.093 | 34.066 | 60.123 | 1.00 | 21.78 | O |
| ATOM | 1975 | CB | THR | A | 572 | 15.030 | 36.832 | 59.248 | 1.00 | 21.77 | C |
| ATOM | 1976 | OG1 | THR | A | 572 | 16.008 | 37.852 | 59.014 | 1.00 | 22.63 | O |
| ATOM | 1977 | CG2 | THR | A | 572 | 13.658 | 37.496 | 59.431 | 1.00 | 21.73 | C |
| ATOM | 1978 | N | GLY | A | 573 | 13.725 | 35.240 | 62.025 | 1.00 | 21.63 | N |
| ATOM | 1979 | CA | GLY | A | 573 | 12.659 | 34.365 | 62.527 | 1.00 | 21.49 | C |
| ATOM | 1980 | C | GLY | A | 573 | 11.338 | 34.645 | 61.836 | 1.00 | 21.48 | C |
| ATOM | 1981 | O | GLY | A | 573 | 11.267 | 35.518 | 60.962 | 1.00 | 21.46 | O |
| ATOM | 1982 | N | ILE | A | 574 | 10.291 | 33.912 | 62.223 | 1.00 | 21.21 | N |
| ATOM | 1983 | CA | ILE | A | 574 | 8.959 | 34.090 | 61.626 | 1.00 | 21.09 | C |
| ATOM | 1984 | C | ILE | A | 574 | 7.836 | 34.216 | 62.652 | 1.00 | 21.28 | C |
| ATOM | 1985 | O | ILE | A | 574 | 7.971 | 33.786 | 63.801 | 1.00 | 21.42 | O |
| ATOM | 1986 | CB | ILE | A | 574 | 8.587 | 32.940 | 60.639 | 1.00 | 20.99 | C |
| ATOM | 1987 | CG1 | ILE | A | 574 | 8.234 | 31.657 | 61.409 | 1.00 | 20.81 | C |
| ATOM | 1988 | CG2 | ILE | A | 574 | 9.695 | 32.739 | 59.595 | 1.00 | 20.50 | C |
| ATOM | 1989 | CD1 | ILE | A | 574 | 7.649 | 30.541 | 60.573 | 1.00 | 20.89 | C |
| ATOM | 1990 | N | VAL | A | 575 | 6.728 | 34.817 | 62.222 | 1.00 | 21.33 | N |
| ATOM | 1991 | CA | VAL | A | 575 | 5.475 | 34.772 | 62.970 | 1.00 | 21.17 | C |
| ATOM | 1992 | C | VAL | A | 575 | 4.517 | 33.868 | 62.190 | 1.00 | 21.11 | C |
| ATOM | 1993 | O | VAL | A | 575 | 4.367 | 34.031 | 60.983 | 1.00 | 20.97 | O |
| ATOM | 1994 | CB | VAL | A | 575 | 4.885 | 36.192 | 63.195 | 1.00 | 21.17 | C |
| ATOM | 1995 | CG1 | VAL | A | 575 | 3.508 | 36.129 | 63.864 | 1.00 | 21.09 | C |
| ATOM | 1996 | CG2 | VAL | A | 575 | 5.829 | 37.022 | 64.041 | 1.00 | 20.92 | C |
| ATOM | 1997 | N | LEU | A | 576 | 3.907 | 32.895 | 62.864 | 1.00 | 21.34 | N |
| ATOM | 1998 | CA | LEU | A | 576 | 2.966 | 31.990 | 62.192 | 1.00 | 21.68 | C |
| ATOM | 1999 | C | LEU | A | 576 | 1.737 | 32.767 | 61.721 | 1.00 | 22.12 | C |
| ATOM | 2000 | O | LEU | A | 576 | 1.121 | 33.507 | 62.496 | 1.00 | 21.91 | O |
| ATOM | 2001 | CB | LEU | A | 576 | 2.566 | 30.807 | 63.087 | 1.00 | 21.37 | C |
| ATOM | 2002 | CG | LEU | A | 576 | 3.671 | 29.884 | 63.622 | 1.00 | 20.82 | C |
| ATOM | 2003 | CD1 | LEU | A | 576 | 3.082 | 28.812 | 64.518 | 1.00 | 19.76 | C |
| ATOM | 2004 | CD2 | LEU | A | 576 | 4.488 | 29.251 | 62.500 | 1.00 | 19.90 | C |
| ATOM | 2005 | N | ARG | A | 577 | 1.409 | 32.610 | 60.441 | 1.00 | 22.62 | N |
| ATOM | 2006 | CA | ARG | A | 577 | 0.344 | 33.387 | 59.809 | 1.00 | 23.46 | C |
| ATOM | 2007 | C | ARG | A | 577 | −1.004 | 32.660 | 59.707 | 1.00 | 23.61 | C |
| ATOM | 2008 | O | ARG | A | 577 | −2.019 | 33.291 | 59.406 | 1.00 | 23.71 | O |
| ATOM | 2009 | CB | ARG | A | 577 | 0.792 | 33.882 | 58.430 | 1.00 | 23.57 | C |
| ATOM | 2010 | CG | ARG | A | 577 | 1.220 | 32.771 | 57.508 | 1.00 | 24.18 | C |
| ATOM | 2011 | CD | ARG | A | 577 | 1.674 | 33.259 | 56.134 | 1.00 | 24.14 | C |
| ATOM | 2012 | NE | ARG | A | 577 | 1.772 | 32.096 | 55.251 | 1.00 | 25.19 | N |
| ATOM | 2013 | CZ | ARG | A | 577 | 2.807 | 31.261 | 55.211 | 1.00 | 24.64 | C |
| ATOM | 2014 | NH1 | ARG | A | 577 | 3.874 | 31.463 | 55.975 | 1.00 | 24.29 | N |
| ATOM | 2015 | NH2 | ARG | A | 577 | 2.777 | 30.223 | 54.394 | 1.00 | 25.16 | N |
| ATOM | 2016 | N | ASN | A | 578 | −1.020 | 31.352 | 59.969 | 1.00 | 23.82 | N |
| ATOM | 2017 | CA | ASN | A | 578 | −2.255 | 30.566 | 59.874 | 1.00 | 24.26 | C |
| ATOM | 2018 | C | ASN | A | 578 | −2.317 | 29.348 | 60.813 | 1.00 | 24.58 | C |
| ATOM | 2019 | O | ASN | A | 578 | −1.302 | 28.932 | 61.373 | 1.00 | 24.56 | O |
| ATOM | 2020 | CB | ASN | A | 578 | −2.493 | 30.137 | 58.423 | 1.00 | 24.09 | C |
| ATOM | 2021 | CG | ASN | A | 578 | −1.512 | 29.092 | 57.960 | 1.00 | 24.12 | C |
| ATOM | 2022 | OD1 | ASN | A | 578 | −1.507 | 27.966 | 58.463 | 1.00 | 25.12 | O |
| ATOM | 2023 | ND2 | ASN | A | 578 | −0.679 | 29.448 | 56.990 | 1.00 | 23.31 | N |
| ATOM | 2024 | N | GLY | A | 579 | −3.510 | 28.773 | 60.964 | 1.00 | 24.89 | N |
| ATOM | 2025 | CA | GLY | A | 579 | −3.706 | 27.614 | 61.836 | 1.00 | 25.08 | C |
| ATOM | 2026 | C | GLY | A | 579 | −3.953 | 28.038 | 63.275 | 1.00 | 25.66 | C |
| ATOM | 2027 | O | GLY | A | 579 | −4.004 | 29.240 | 63.571 | 1.00 | 25.92 | O |
| ATOM | 2028 | N | PRO | A | 580 | −4.108 | 27.060 | 64.186 | 1.00 | 25.76 | N |
| ATOM | 2029 | CA | PRO | A | 580 | −4.485 | 27.359 | 65.575 | 1.00 | 25.83 | C |
| ATOM | 2030 | C | PRO | A | 580 | −3.392 | 28.005 | 66.439 | 1.00 | 25.99 | C |
| ATOM | 2031 | O | PRO | A | 580 | −3.665 | 28.376 | 67.587 | 1.00 | 26.04 | O |
| ATOM | 2032 | CB | PRO | A | 580 | −4.888 | 25.988 | 66.137 | 1.00 | 25.87 | C |
| ATOM | 2033 | CG | PRO | A | 580 | −4.175 | 24.996 | 65.290 | 1.00 | 25.83 | C |
| ATOM | 2034 | CD | PRO | A | 580 | −3.958 | 25.613 | 63.941 | 1.00 | 25.80 | C |
| ATOM | 2035 | N | HIS | A | 581 | −2.180 | 28.144 | 65.900 | 1.00 | 25.90 | N |
| ATOM | 2036 | CA | HIS | A | 581 | −1.070 | 28.768 | 66.635 | 1.00 | 25.74 | C |
| ATOM | 2037 | C | HIS | A | 581 | −0.619 | 30.080 | 65.980 | 1.00 | 25.62 | C |
| ATOM | 2038 | O | HIS | A | 581 | 0.438 | 30.623 | 66.307 | 1.00 | 25.64 | O |
| ATOM | 2039 | CB | HIS | A | 581 | 0.106 | 27.796 | 66.771 | 1.00 | 25.66 | C |
| ATOM | 2040 | CG | HIS | A | 581 | −0.269 | 26.468 | 67.358 | 1.00 | 26.37 | C |
| ATOM | 2041 | ND1 | HIS | A | 581 | −0.164 | 25.284 | 66.656 | 1.00 | 26.76 | N |
| ATOM | 2042 | CD2 | HIS | A | 581 | −0.746 | 26.136 | 68.582 | 1.00 | 26.38 | C |
| ATOM | 2043 | CE1 | HIS | A | 581 | −0.568 | 24.284 | 67.418 | 1.00 | 26.70 | C |
| ATOM | 2044 | NE2 | HIS | A | 581 | −0.924 | 24.773 | 68.593 | 1.00 | 27.30 | N |
| ATOM | 2045 | N | LYS | A | 582 | −1.439 | 30.572 | 65.052 | 1.00 | 25.31 | N |
| ATOM | 2046 | CA | LYS | A | 582 | −1.219 | 31.841 | 64.358 | 1.00 | 24.93 | C |
| ATOM | 2047 | C | LYS | A | 582 | −0.881 | 32.950 | 65.346 | 1.00 | 24.26 | C |
| ATOM | 2048 | O | LYS | A | 582 | −1.552 | 33.107 | 66.366 | 1.00 | 24.34 | O |
| ATOM | 2049 | CB | LYS | A | 582 | −2.469 | 32.195 | 63.542 | 1.00 | 24.85 | C |
| ATOM | 2050 | CG | LYS | A | 582 | −2.434 | 33.516 | 62.776 | 1.00 | 25.44 | C |
| ATOM | 2051 | CD | LYS | A | 582 | −3.838 | 33.829 | 62.233 | 1.00 | 25.80 | C |
| ATOM | 2052 | CE | LYS | A | 582 | −3.886 | 35.105 | 61.390 | 1.00 | 27.57 | C |
| ATOM | 2053 | NZ | LYS | A | 582 | −3.303 | 36.285 | 62.075 | 1.00 | 28.30 | N |
| ATOM | 2054 | N | GLY | A | 583 | 0.169 | 33.707 | 65.043 | 1.00 | 23.76 | N |
| ATOM | 2055 | CA | GLY | A | 583 | 0.624 | 34.783 | 65.917 | 1.00 | 22.91 | C |
| ATOM | 2056 | C | GLY | A | 583 | 1.778 | 34.356 | 66.808 | 1.00 | 22.58 | C |
| ATOM | 2057 | O | GLY | A | 583 | 2.388 | 35.192 | 67.477 | 1.00 | 22.36 | O |
| ATOM | 2058 | N | ARG | A | 584 | 2.063 | 33.052 | 66.830 | 1.00 | 22.27 | N |
| ATOM | 2059 | CA | ARG | A | 584 | 3.231 | 32.517 | 67.538 | 1.00 | 21.91 | C |
| ATOM | 2060 | C | ARG | A | 584 | 4.535 | 32.937 | 66.861 | 1.00 | 21.53 | C |
| ATOM | 2061 | O | ARG | A | 584 | 4.687 | 32.809 | 65.641 | 1.00 | 21.33 | O |
| ATOM | 2062 | CB | ARG | A | 584 | 3.166 | 30.994 | 67.622 | 1.00 | 21.98 | C |
| ATOM | 2063 | CG | ARG | A | 584 | 4.369 | 30.351 | 68.291 | 1.00 | 22.52 | C |
| ATOM | 2064 | CD | ARG | A | 584 | 4.218 | 28.853 | 68.360 | 1.00 | 22.64 | C |
| ATOM | 2065 | NE | ARG | A | 584 | 3.297 | 28.458 | 69.421 | 1.00 | 23.70 | N |
| ATOM | 2066 | CZ | ARG | A | 584 | 2.758 | 27.246 | 69.530 | 1.00 | 23.67 | C |
| ATOM | 2067 | NH1 | ARG | A | 584 | 3.039 | 26.302 | 68.640 | 1.00 | 22.10 | N |
| ATOM | 2068 | NH2 | ARG | A | 584 | 1.931 | 26.982 | 70.533 | 1.00 | 24.49 | N |
| ATOM | 2069 | N | ILE | A | 585 | 5.463 | 33.435 | 67.672 | 1.00 | 20.94 | N |
| ATOM | 2070 | CA | ILE | A | 585 | 6.784 | 33.823 | 67.207 | 1.00 | 20.71 | C |
| ATOM | 2071 | C | ILE | A | 585 | 7.712 | 32.618 | 67.297 | 1.00 | 20.41 | C |
| ATOM | 2072 | O | ILE | A | 585 | 7.761 | 31.962 | 68.327 | 1.00 | 20.72 | O |
| ATOM | 2073 | CB | ILE | A | 585 | 7.359 | 34.989 | 68.049 | 1.00 | 20.62 | C |
| ATOM | 2074 | CG1 | ILE | A | 585 | 6.416 | 36.198 | 68.002 | 1.00 | 20.69 | C |
| ATOM | 2075 | CG2 | ILE | A | 585 | 8.769 | 35.355 | 67.578 | 1.00 | 20.07 | C |
| ATOM | 2076 | CD1 | ILE | A | 585 | 6.782 | 37.323 | 68.971 | 1.00 | 21.07 | C |
| ATOM | 2077 | N | LEU | A | 586 | 8.430 | 32.322 | 66.217 | 1.00 | 20.13 | N |
| ATOM | 2078 | CA | LEU | A | 586 | 9.403 | 31.229 | 66.214 | 1.00 | 19.91 | C |
| ATOM | 2079 | C | LEU | A | 586 | 10.822 | 31.743 | 66.014 | 1.00 | 19.80 | C |
| ATOM | 2080 | O | LEU | A | 586 | 11.101 | 32.484 | 65.063 | 1.00 | 20.11 | O |
| ATOM | 2081 | CB | LEU | A | 586 | 9.071 | 30.190 | 65.137 | 1.00 | 19.96 | C |
| ATOM | 2082 | CG | LEU | A | 586 | 7.706 | 29.494 | 65.164 | 1.00 | 19.90 | C |
| ATOM | 2083 | CD1 | LEU | A | 586 | 7.648 | 28.439 | 64.074 | 1.00 | 19.88 | C |
| ATOM | 2084 | CD2 | LEU | A | 586 | 7.437 | 28.874 | 66.517 | 1.00 | 20.06 | C |
| ATOM | 2085 | N | ILE | A | 587 | 11.704 | 31.351 | 66.927 | 1.00 | 19.17 | N |
| ATOM | 2086 | CA | ILE | A | 587 | 13.117 | 31.688 | 66.863 | 1.00 | 18.50 | C |
| ATOM | 2087 | C | ILE | A | 587 | 13.894 | 30.384 | 66.806 | 1.00 | 18.40 | C |
| ATOM | 2088 | O | ILE | A | 587 | 13.836 | 29.593 | 67.743 | 1.00 | 18.34 | O |
| ATOM | 2089 | CB | ILE | A | 587 | 13.576 | 32.476 | 68.120 | 1.00 | 18.32 | C |
| ATOM | 2090 | CG1 | ILE | A | 587 | 12.623 | 33.640 | 68.441 | 1.00 | 18.13 | C |
| ATOM | 2091 | CG2 | ILE | A | 587 | 15.024 | 32.918 | 67.982 | 1.00 | 17.75 | C |
| ATOM | 2092 | CD1 | ILE | A | 587 | 12.569 | 34.754 | 67.398 | 1.00 | 17.34 | C |
| ATOM | 2093 | N | PRO | A | 588 | 14.602 | 30.134 | 65.696 | 1.00 | 18.50 | N |
| ATOM | 2094 | CA | PRO | A | 588 | 15.435 | 28.927 | 65.607 | 1.00 | 18.31 | C |
| ATOM | 2095 | C | PRO | A | 588 | 16.582 | 28.971 | 66.614 | 1.00 | 18.11 | C |
| ATOM | 2096 | O | PRO | A | 588 | 17.202 | 30.028 | 66.804 | 1.00 | 18.36 | O |
| ATOM | 2097 | CB | PRO | A | 588 | 15.989 | 28.982 | 64.177 | 1.00 | 18.21 | C |
| ATOM | 2098 | CG | PRO | A | 588 | 15.062 | 29.917 | 63.433 | 1.00 | 18.51 | C |
| ATOM | 2099 | CD | PRO | A | 588 | 14.654 | 30.932 | 64.457 | 1.00 | 18.57 | C |
| ATOM | 2100 | N | VAL | A | 589 | 16.836 | 27.840 | 67.266 | 1.00 | 17.61 | N |
| ATOM | 2101 | CA | VAL | A | 589 | 17.930 | 27.711 | 68.224 | 1.00 | 17.23 | C |
| ATOM | 2102 | C | VAL | A | 589 | 18.545 | 26.311 | 68.147 | 1.00 | 17.10 | C |
| ATOM | 2103 | O | VAL | A | 589 | 18.024 | 25.423 | 67.464 | 1.00 | 16.74 | O |
| ATOM | 2104 | CB | VAL | A | 589 | 17.481 | 27.987 | 69.693 | 1.00 | 17.17 | C |
| ATOM | 2105 | CG1 | VAL | A | 589 | 16.866 | 29.376 | 69.851 | 1.00 | 17.28 | C |
| ATOM | 2106 | CG2 | VAL | A | 589 | 16.514 | 26.927 | 70.184 | 1.00 | 17.84 | C |
| ATOM | 2107 | N | TYR | A | 590 | 19.655 | 26.122 | 68.852 | 1.00 | 17.03 | N |
| ATOM | 2108 | CA | TYR | A | 590 | 20.240 | 24.801 | 69.003 | 1.00 | 17.44 | C |
| ATOM | 2109 | C | TYR | A | 590 | 21.027 | 24.646 | 70.291 | 1.00 | 17.67 | C |
| ATOM | 2110 | O | TYR | A | 590 | 21.468 | 25.628 | 70.891 | 1.00 | 17.46 | O |
| ATOM | 2111 | CB | TYR | A | 590 | 21.073 | 24.394 | 67.777 | 1.00 | 17.27 | C |
| ATOM | 2112 | CG | TYR | A | 590 | 22.323 | 25.194 | 67.535 | 1.00 | 17.42 | C |
| ATOM | 2113 | CD1 | TYR | A | 590 | 22.260 | 26.497 | 67.018 | 1.00 | 17.48 | C |
| ATOM | 2114 | CD2 | TYR | A | 590 | 23.575 | 24.640 | 67.781 | 1.00 | 16.63 | C |
| ATOM | 2115 | CE1 | TYR | A | 590 | 23.419 | 27.233 | 66.776 | 1.00 | 16.90 | C |
| ATOM | 2116 | CE2 | TYR | A | 590 | 24.736 | 25.360 | 67.537 | 1.00 | 17.04 | C |
| ATOM | 2117 | CZ | TYR | A | 590 | 24.652 | 26.655 | 67.034 | 1.00 | 17.25 | C |
| ATOM | 2118 | OH | TYR | A | 590 | 25.810 | 27.366 | 66.795 | 1.00 | 18.08 | O |
| ATOM | 2119 | N | THR | A | 591 | 21.188 | 23.393 | 70.706 | 1.00 | 18.22 | N |
| ATOM | 2120 | CA | THR | A | 591 | 21.793 | 23.069 | 71.992 | 1.00 | 18.65 | C |
| ATOM | 2121 | C | THR | A | 591 | 22.931 | 22.063 | 71.860 | 1.00 | 19.26 | C |
| ATOM | 2122 | O | THR | A | 591 | 23.021 | 21.335 | 70.866 | 1.00 | 19.63 | O |
| ATOM | 2123 | CB | THR | A | 591 | 20.752 | 22.470 | 72.969 | 1.00 | 18.43 | C |
| ATOM | 2124 | OG1 | THR | A | 591 | 20.208 | 21.269 | 72.408 | 1.00 | 18.34 | O |
| ATOM | 2125 | CG2 | THR | A | 591 | 19.627 | 23.456 | 73.252 | 1.00 | 17.97 | C |
| ATOM | 2126 | N | THR | A | 592 | 23.800 | 22.039 | 72.868 | 1.00 | 19.51 | N |
| ATOM | 2127 | CA | THR | A | 592 | 24.727 | 20.938 | 73.065 | 1.00 | 19.73 | C |
| ATOM | 2128 | C | THR | A | 592 | 24.473 | 20.380 | 74.457 | 1.00 | 20.07 | C |
| ATOM | 2129 | O | THR | A | 592 | 23.892 | 21.065 | 75.314 | 1.00 | 19.74 | O |
| ATOM | 2130 | CB | THR | A | 592 | 26.210 | 21.369 | 72.935 | 1.00 | 19.96 | C |
| ATOM | 2131 | OG1 | THR | A | 592 | 26.581 | 22.192 | 74.049 | 1.00 | 20.26 | O |
| ATOM | 2132 | CG2 | THR | A | 592 | 26.450 | 22.130 | 71.635 | 1.00 | 19.54 | C |
| ATOM | 2133 | N | ASN | A | 593 | 24.878 | 19.131 | 74.673 | 1.00 | 20.44 | N |
| ATOM | 2134 | CA | ASN | A | 593 | 24.787 | 18.514 | 75.992 | 1.00 | 20.95 | C |
| ATOM | 2135 | C | ASN | A | 593 | 26.169 | 18.265 | 76.582 | 1.00 | 21.40 | C |
| ATOM | 2136 | O | ASN | A | 593 | 27.185 | 18.496 | 75.918 | 1.00 | 21.65 | O |
| ATOM | 2137 | CB | ASN | A | 593 | 23.958 | 17.224 | 75.953 | 1.00 | 20.88 | C |
| ATOM | 2138 | CG | ASN | A | 593 | 24.593 | 16.137 | 75.112 | 1.00 | 20.73 | C |
| ATOM | 2139 | OD1 | ASN | A | 593 | 25.815 | 16.024 | 75.033 | 1.00 | 21.63 | O |
| ATOM | 2140 | ND2 | ASN | A | 593 | 23.760 | 15.324 | 74.479 | 1.00 | 19.73 | N |
| ATOM | 2141 | N | ASN | A | 594 | 26.201 | 17.788 | 77.823 | 1.00 | 21.73 | N |
| ATOM | 2142 | CA | ASN | A | 594 | 27.455 | 17.592 | 78.546 | 1.00 | 21.83 | C |
| ATOM | 2143 | C | ASN | A | 594 | 28.127 | 16.264 | 78.212 | 1.00 | 22.25 | C |
| ATOM | 2144 | O | ASN | A | 594 | 29.251 | 16.004 | 78.642 | 1.00 | 22.47 | O |
| ATOM | 2145 | CB | ASN | A | 594 | 27.224 | 17.724 | 80.057 | 1.00 | 21.54 | C |
| ATOM | 2146 | CG | ASN | A | 594 | 26.924 | 19.156 | 80.486 | 1.00 | 21.49 | C |
| ATOM | 2147 | OD1 | ASN | A | 594 | 27.625 | 20.101 | 80.101 | 1.00 | 21.64 | O |
| ATOM | 2148 | ND2 | ASN | A | 594 | 25.891 | 19.322 | 81.306 | 1.00 | 21.20 | N |
| ATOM | 2149 | N | VAL | A | 595 | 27.437 | 15.427 | 77.439 | 1.00 | 22.76 | N |
| ATOM | 2150 | CA | VAL | A | 595 | 27.965 | 14.116 | 77.061 | 1.00 | 23.03 | C |
| ATOM | 2151 | C | VAL | A | 595 | 28.971 | 14.242 | 75.929 | 1.00 | 23.41 | C |
| ATOM | 2152 | O | VAL | A | 595 | 30.038 | 13.623 | 75.976 | 1.00 | 23.84 | O |
| ATOM | 2153 | CB | VAL | A | 595 | 26.838 | 13.099 | 76.694 | 1.00 | 23.07 | C |
| ATOM | 2154 | CG1 | VAL | A | 595 | 27.423 | 11.773 | 76.170 | 1.00 | 22.63 | C |
| ATOM | 2155 | CG2 | VAL | A | 595 | 25.962 | 12.826 | 77.898 | 1.00 | 22.53 | C |
| ATOM | 2156 | N | SER | A | 596 | 28.644 | 15.047 | 74.921 | 1.00 | 23.60 | N |
| ATOM | 2157 | CA | SER | A | 596 | 29.529 | 15.195 | 73.761 | 1.00 | 23.84 | C |
| ATOM | 2158 | C | SER | A | 596 | 29.772 | 16.637 | 73.285 | 1.00 | 23.96 | C |
| ATOM | 2159 | O | SER | A | 596 | 30.584 | 16.858 | 72.395 | 1.00 | 24.21 | O |
| ATOM | 2160 | CB | SER | A | 596 | 29.043 | 14.316 | 72.600 | 1.00 | 23.74 | C |
| ATOM | 2161 | OG | SER | A | 596 | 27.714 | 14.631 | 72.233 | 1.00 | 23.37 | O |
| ATOM | 2162 | N | HIS | A | 597 | 29.083 | 17.605 | 73.886 | 1.00 | 24.27 | N |
| ATOM | 2163 | CA | HIS | A | 597 | 29.221 | 19.035 | 73.535 | 1.00 | 24.28 | C |
| ATOM | 2164 | C | HIS | A | 597 | 29.168 | 19.301 | 72.014 | 1.00 | 24.53 | C |
| ATOM | 2165 | O | HIS | A | 597 | 28.119 | 19.102 | 71.396 | 1.00 | 24.93 | O |
| ATOM | 2166 | CB | HIS | A | 597 | 30.456 | 19.656 | 74.208 | 1.00 | 24.04 | C |
| ATOM | 2167 | CG | HIS | A | 597 | 30.575 | 21.136 | 74.017 | 1.00 | 24.17 | C |
| ATOM | 2168 | ND1 | HIS | A | 597 | 29.504 | 21.996 | 74.152 | 1.00 | 24.60 | N |
| ATOM | 2169 | CD2 | HIS | A | 597 | 31.642 | 21.910 | 73.710 | 1.00 | 23.89 | C |
| ATOM | 2170 | CE1 | HIS | A | 597 | 29.906 | 23.235 | 73.928 | 1.00 | 24.14 | C |
| ATOM | 2171 | NE2 | HIS | A | 597 | 31.199 | 23.210 | 73.658 | 1.00 | 24.20 | N |
| ATOM | 2172 | N | LEU | A | 598 | 30.285 | 19.723 | 71.418 | 1.00 | 24.22 | N |
| ATOM | 2173 | CA | LEU | A | 598 | 30.323 | 20.075 | 70.002 | 1.00 | 23.93 | C |
| ATOM | 2174 | C | LEU | A | 598 | 30.669 | 18.895 | 69.092 | 1.00 | 24.07 | C |
| ATOM | 2175 | O | LEU | A | 598 | 30.557 | 18.997 | 67.866 | 1.00 | 23.97 | O |
| ATOM | 2176 | CB | LEU | A | 598 | 31.290 | 21.239 | 69.759 | 1.00 | 23.70 | C |
| ATOM | 2177 | CG | LEU | A | 598 | 30.799 | 22.639 | 70.152 | 1.00 | 23.89 | C |
| ATOM | 2178 | CD1 | LEU | A | 598 | 31.941 | 23.638 | 70.137 | 1.00 | 23.90 | C |
| ATOM | 2179 | CD2 | LEU | A | 598 | 29.666 | 23.119 | 69.243 | 1.00 | 23.37 | C |
| ATOM | 2180 | N | ASN | A | 599 | 31.081 | 17.779 | 69.689 | 1.00 | 23.95 | N |
| ATOM | 2181 | CA | ASN | A | 599 | 31.466 | 16.603 | 68.916 | 1.00 | 24.06 | C |
| ATOM | 2182 | C | ASN | A | 599 | 30.305 | 15.806 | 68.332 | 1.00 | 23.68 | C |
| ATOM | 2183 | O | ASN | A | 599 | 30.387 | 15.356 | 67.188 | 1.00 | 23.88 | O |
| ATOM | 2184 | CB | ASN | A | 599 | 32.423 | 15.707 | 69.705 | 1.00 | 24.38 | C |
| ATOM | 2185 | CG | ASN | A | 599 | 33.880 | 16.134 | 69.538 | 1.00 | 25.66 | C |
| ATOM | 2186 | OD1 | ASN | A | 599 | 34.585 | 15.634 | 68.655 | 1.00 | 27.02 | O |
| ATOM | 2187 | ND2 | ASN | A | 599 | 34.323 | 17.085 | 70.358 | 1.00 | 25.44 | N |
| ATOM | 2188 | N | GLY | A | 600 | 29.229 | 15.638 | 69.099 | 1.00 | 23.13 | N |
| ATOM | 2189 | CA | GLY | A | 600 | 28.077 | 14.886 | 68.621 | 1.00 | 22.08 | C |
| ATOM | 2190 | C | GLY | A | 600 | 26.734 | 15.124 | 69.285 | 1.00 | 21.59 | C |
| ATOM | 2191 | O | GLY | A | 600 | 25.932 | 14.194 | 69.378 | 1.00 | 21.68 | O |
| ATOM | 2192 | N | SER | A | 601 | 26.462 | 16.354 | 69.721 | 1.00 | 21.04 | N |
| ATOM | 2193 | CA | SER | A | 601 | 25.170 | 16.655 | 70.356 | 1.00 | 20.62 | C |
| ATOM | 2194 | C | SER | A | 601 | 24.442 | 17.895 | 69.843 | 1.00 | 20.40 | C |
| ATOM | 2195 | O | SER | A | 601 | 23.375 | 18.245 | 70.361 | 1.00 | 20.39 | O |
| ATOM | 2196 | CB | SER | A | 601 | 25.312 | 16.749 | 71.876 | 1.00 | 20.53 | C |
| ATOM | 2197 | OG | SER | A | 601 | 25.867 | 17.992 | 72.267 | 1.00 | 20.21 | O |
| ATOM | 2198 | N | GLN | A | 602 | 25.001 | 18.565 | 68.844 | 1.00 | 20.05 | N |
| ATOM | 2199 | CA | GLN | A | 602 | 24.341 | 19.750 | 68.296 | 1.00 | 20.01 | C |
| ATOM | 2200 | C | GLN | A | 602 | 22.926 | 19.411 | 67.828 | 1.00 | 19.79 | C |
| ATOM | 2201 | O | GLN | A | 602 | 22.733 | 18.530 | 66.993 | 1.00 | 20.05 | O |
| ATOM | 2202 | CB | GLN | A | 602 | 25.175 | 20.386 | 67.190 | 1.00 | 19.72 | C |
| ATOM | 2203 | CG | GLN | A | 602 | 26.334 | 21.208 | 67.727 | 1.00 | 19.95 | C |
| ATOM | 2204 | CD | GLN | A | 602 | 27.285 | 21.674 | 66.642 | 1.00 | 20.59 | C |
| ATOM | 2205 | OE1 | GLN | A | 602 | 27.205 | 22.816 | 66.183 | 1.00 | 21.19 | O |
| ATOM | 2206 | NE2 | GLN | A | 602 | 28.191 | 20.791 | 66.224 | 1.00 | 19.21 | N |
| ATOM | 2207 | N | SER | A | 603 | 21.936 | 20.089 | 68.401 | 1.00 | 19.56 | N |
| ATOM | 2208 | CA | SER | A | 603 | 20.533 | 19.733 | 68.167 | 1.00 | 19.37 | C |
| ATOM | 2209 | C | SER | A | 603 | 19.646 | 20.949 | 67.957 | 1.00 | 19.29 | C |
| ATOM | 2210 | O | SER | A | 603 | 19.533 | 21.806 | 68.838 | 1.00 | 19.38 | O |
| ATOM | 2211 | CB | SER | A | 603 | 20.000 | 18.879 | 69.319 | 1.00 | 19.30 | C |
| ATOM | 2212 | OG | SER | A | 603 | 20.629 | 17.604 | 69.340 | 1.00 | 18.84 | O |
| ATOM | 2213 | N | SER | A | 604 | 19.023 | 21.007 | 66.783 | 1.00 | 19.00 | N |
| ATOM | 2214 | CA | SER | A | 604 | 18.105 | 22.080 | 66.418 | 1.00 | 18.78 | C |
| ATOM | 2215 | C | SER | A | 604 | 16.766 | 21.994 | 67.142 | 1.00 | 18.70 | C |
| ATOM | 2216 | O | SER | A | 604 | 16.241 | 20.900 | 67.395 | 1.00 | 18.51 | O |
| ATOM | 2217 | CB | SER | A | 604 | 17.835 | 22.055 | 64.912 | 1.00 | 18.85 | C |
| ATOM | 2218 | OG | SER | A | 604 | 19.034 | 22.112 | 64.167 | 1.00 | 18.95 | O |
| ATOM | 2219 | N | ARG | A | 605 | 16.227 | 23.165 | 67.471 | 1.00 | 18.56 | N |
| ATOM | 2220 | CA | ARG | A | 605 | 14.838 | 23.306 | 67.904 | 1.00 | 18.27 | C |
| ATOM | 2221 | C | ARG | A | 605 | 14.392 | 24.749 | 67.676 | 1.00 | 18.52 | C |
| ATOM | 2222 | O | ARG | A | 605 | 15.148 | 25.557 | 67.127 | 1.00 | 18.41 | O |
| ATOM | 2223 | CB | ARG | A | 605 | 14.653 | 22.882 | 69.376 | 1.00 | 18.20 | C |
| ATOM | 2224 | CG | ARG | A | 605 | 15.318 | 23.780 | 70.422 | 1.00 | 17.99 | C |
| ATOM | 2225 | CD | ARG | A | 605 | 14.961 | 23.349 | 71.850 | 1.00 | 17.59 | C |
| ATOM | 2226 | NE | ARG | A | 605 | 15.634 | 22.106 | 72.209 | 1.00 | 16.04 | N |
| ATOM | 2227 | CZ | ARG | A | 605 | 15.308 | 21.317 | 73.230 | 1.00 | 16.40 | C |
| ATOM | 2228 | NH1 | ARG | A | 605 | 14.291 | 21.613 | 74.034 | 1.00 | 14.56 | N |
| ATOM | 2229 | NH2 | ARG | A | 605 | 16.014 | 20.213 | 73.448 | 1.00 | 16.98 | N |
| ATOM | 2230 | N | ILE | A | 606 | 13.167 | 25.069 | 68.086 | 1.00 | 18.50 | N |
| ATOM | 2231 | CA | ILE | A | 606 | 12.734 | 26.456 | 68.146 | 1.00 | 18.74 | C |
| ATOM | 2232 | C | ILE | A | 606 | 12.387 | 26.840 | 69.581 | 1.00 | 18.93 | C |
| ATOM | 2233 | O | ILE | A | 606 | 12.044 | 25.984 | 70.396 | 1.00 | 19.22 | O |
| ATOM | 2234 | CB | ILE | A | 606 | 11.528 | 26.755 | 67.195 | 1.00 | 18.61 | C |
| ATOM | 2235 | CG1 | ILE | A | 606 | 10.289 | 25.927 | 67.567 | 1.00 | 19.01 | C |
| ATOM | 2236 | CG2 | ILE | A | 606 | 11.909 | 26.504 | 65.735 | 1.00 | 18.48 | C |
| ATOM | 2237 | CD1 | ILE | A | 606 | 9.402 | 26.540 | 68.640 | 1.00 | 18.43 | C |
| ATOM | 2238 | N | ILE | A | 607 | 12.507 | 28.126 | 69.887 | 1.00 | 19.08 | N |
| ATOM | 2239 | CA | ILE | A | 607 | 11.836 | 28.695 | 71.040 | 1.00 | 19.35 | C |
| ATOM | 2240 | C | ILE | A | 607 | 10.726 | 29.570 | 70.480 | 1.00 | 19.74 | C |
| ATOM | 2241 | O | ILE | A | 607 | 10.815 | 30.026 | 69.337 | 1.00 | 19.52 | O |
| ATOM | 2242 | CB | ILE | A | 607 | 12.778 | 29.493 | 72.008 | 1.00 | 19.29 | C |
| ATOM | 2243 | CG1 | ILE | A | 607 | 13.474 | 30.659 | 71.299 | 1.00 | 19.06 | C |
| ATOM | 2244 | CG2 | ILE | A | 607 | 13.784 | 28.558 | 72.674 | 1.00 | 19.19 | C |
| ATOM | 2245 | CD1 | ILE | A | 607 | 14.048 | 31.712 | 72.243 | 1.00 | 19.16 | C |
| ATOM | 2246 | N | TYR | A | 608 | 9.680 | 29.779 | 71.276 | 1.00 | 20.27 | N |
| ATOM | 2247 | CA | TYR | A | 608 | 8.483 | 30.468 | 70.814 | 1.00 | 20.83 | C |
| ATOM | 2248 | C | TYR | A | 608 | 7.835 | 31.353 | 71.878 | 1.00 | 21.25 | C |
| ATOM | 2249 | O | TYR | A | 608 | 8.082 | 31.186 | 73.077 | 1.00 | 21.22 | O |
| ATOM | 2250 | CB | TYR | A | 608 | 7.469 | 29.452 | 70.258 | 1.00 | 21.21 | C |
| ATOM | 2251 | CG | TYR | A | 608 | 6.839 | 28.527 | 71.290 | 1.00 | 21.19 | C |
| ATOM | 2252 | CD1 | TYR | A | 608 | 7.448 | 27.322 | 71.639 | 1.00 | 21.34 | C |
| ATOM | 2253 | CD2 | TYR | A | 608 | 5.627 | 28.850 | 71.894 | 1.00 | 20.57 | C |
| ATOM | 2254 | CE1 | TYR | A | 608 | 6.873 | 26.470 | 72.582 | 1.00 | 21.23 | C |
| ATOM | 2255 | CE2 | TYR | A | 608 | 5.042 | 28.006 | 72.831 | 1.00 | 20.89 | C |
| ATOM | 2256 | CZ | TYR | A | 608 | 5.668 | 26.820 | 73.170 | 1.00 | 21.45 | C |
| ATOM | 2257 | OH | TYR | A | 608 | 5.087 | 25.983 | 74.096 | 1.00 | 21.17 | O |
| ATOM | 2258 | N | SER | A | 609 | 7.015 | 32.302 | 71.422 | 1.00 | 21.74 | N |
| ATOM | 2259 | CA | SER | A | 609 | 6.237 | 33.173 | 72.304 | 1.00 | 21.86 | C |
| ATOM | 2260 | C | SER | A | 609 | 4.810 | 33.279 | 71.789 | 1.00 | 22.56 | C |
| ATOM | 2261 | O | SER | A | 609 | 4.586 | 33.523 | 70.600 | 1.00 | 22.56 | O |
| ATOM | 2262 | CB | SER | A | 609 | 6.865 | 34.562 | 72.394 | 1.00 | 21.81 | C |
| ATOM | 2263 | OG | SER | A | 609 | 6.124 | 35.413 | 73.261 | 1.00 | 20.41 | O |
| ATOM | 2264 | N | ASP | A | 610 | 3.849 | 33.082 | 72.684 | 1.00 | 23.33 | N |
| ATOM | 2265 | CA | ASP | A | 610 | 2.432 | 33.217 | 72.334 | 1.00 | 24.10 | C |
| ATOM | 2266 | C | ASP | A | 610 | 1.817 | 34.526 | 72.855 | 1.00 | 24.50 | C |
| ATOM | 2267 | O | ASP | A | 610 | 0.629 | 34.794 | 72.635 | 1.00 | 24.43 | O |
| ATOM | 2268 | CB | ASP | A | 610 | 1.632 | 32.012 | 72.839 | 1.00 | 24.02 | C |
| ATOM | 2269 | CG | ASP | A | 610 | 1.882 | 30.755 | 72.025 | 1.00 | 24.33 | C |
| ATOM | 2270 | OD1 | ASP | A | 610 | 2.239 | 30.851 | 70.826 | 1.00 | 23.74 | O |
| ATOM | 2271 | OD2 | ASP | A | 610 | 1.711 | 29.660 | 72.597 | 1.00 | 24.37 | O |
| ATOM | 2272 | N | ASP | A | 611 | 2.637 | 35.339 | 73.523 | 1.00 | 24.83 | N |
| ATOM | 2273 | CA | ASP | A | 611 | 2.169 | 36.579 | 74.135 | 1.00 | 25.20 | C |
| ATOM | 2274 | C | ASP | A | 611 | 2.961 | 37.802 | 73.682 | 1.00 | 25.48 | C |
| ATOM | 2275 | O | ASP | A | 611 | 3.212 | 38.717 | 74.467 | 1.00 | 25.52 | O |
| ATOM | 2276 | CB | ASP | A | 611 | 2.153 | 36.467 | 75.664 | 1.00 | 25.19 | C |
| ATOM | 2277 | CG | ASP | A | 611 | 3.527 | 36.160 | 76.261 | 1.00 | 25.85 | C |
| ATOM | 2278 | OD1 | ASP | A | 611 | 4.562 | 36.378 | 75.597 | 1.00 | 25.44 | O |
| ATOM | 2279 | OD2 | ASP | A | 611 | 3.562 | 35.712 | 77.428 | 1.00 | 27.33 | O |
| ATOM | 2280 | N | HIS | A | 612 | 3.340 | 37.801 | 72.408 | 1.00 | 25.80 | N |
| ATOM | 2281 | CA | HIS | A | 612 | 3.983 | 38.945 | 71.766 | 1.00 | 26.15 | C |
| ATOM | 2282 | C | HIS | A | 612 | 5.325 | 39.320 | 72.396 | 1.00 | 26.25 | C |
| ATOM | 2283 | O | HIS | A | 612 | 5.649 | 40.503 | 72.524 | 1.00 | 26.40 | O |
| ATOM | 2284 | CB | HIS | A | 612 | 3.024 | 40.146 | 71.710 | 1.00 | 26.40 | C |
| ATOM | 2285 | CG | HIS | A | 612 | 1.667 | 39.810 | 71.162 | 1.00 | 26.89 | C |
| ATOM | 2286 | ND1 | HIS | A | 612 | 1.475 | 39.337 | 69.879 | 1.00 | 25.90 | N |
| ATOM | 2287 | CD2 | HIS | A | 612 | 0.435 | 39.880 | 71.723 | 1.00 | 26.58 | C |
| ATOM | 2288 | CE1 | HIS | A | 612 | 0.187 | 39.129 | 69.675 | 1.00 | 25.20 | C |
| ATOM | 2289 | NE2 | HIS | A | 612 | −0.467 | 39.456 | 70.776 | 1.00 | 26.45 | N |
| ATOM | 2290 | N | GLY | A | 613 | 6.095 | 38.304 | 72.787 | 1.00 | 26.18 | N |
| ATOM | 2291 | CA | GLY | A | 613 | 7.474 | 38.496 | 73.225 | 1.00 | 25.90 | C |
| ATOM | 2292 | C | GLY | A | 613 | 7.694 | 38.656 | 74.716 | 1.00 | 25.90 | C |
| ATOM | 2293 | O | GLY | A | 613 | 8.827 | 38.856 | 75.155 | 1.00 | 25.60 | O |
| ATOM | 2294 | N | LYS | A | 614 | 6.621 | 38.569 | 75.498 | 1.00 | 25.98 | N |
| ATOM | 2295 | CA | LYS | A | 614 | 6.733 | 38.696 | 76.954 | 1.00 | 26.22 | C |
| ATOM | 2296 | C | LYS | A | 614 | 7.397 | 37.468 | 77.592 | 1.00 | 25.55 | C |
| ATOM | 2297 | O | LYS | A | 614 | 8.308 | 37.611 | 78.405 | 1.00 | 25.86 | O |
| ATOM | 2298 | CB | LYS | A | 614 | 5.365 | 38.996 | 77.595 | 1.00 | 26.29 | C |
| ATOM | 2299 | CG | LYS | A | 614 | 5.385 | 39.112 | 79.128 | 1.00 | 27.03 | C |
| ATOM | 2300 | CD | LYS | A | 614 | 4.074 | 39.661 | 79.678 | 1.00 | 27.15 | C |
| ATOM | 2301 | CE | LYS | A | 614 | 4.125 | 39.790 | 81.202 | 1.00 | 29.70 | C |
| ATOM | 2302 | NZ | LYS | A | 614 | 2.817 | 40.248 | 81.793 | 1.00 | 30.30 | N |
| ATOM | 2303 | N | THR | A | 615 | 6.934 | 36.273 | 77.233 | 1.00 | 24.85 | N |
| ATOM | 2304 | CA | THR | A | 615 | 7.526 | 35.030 | 77.740 | 1.00 | 24.31 | C |
| ATOM | 2305 | C | THR | A | 615 | 7.947 | 34.139 | 76.585 | 1.00 | 24.03 | C |
| ATOM | 2306 | O | THR | A | 615 | 7.405 | 34.237 | 75.487 | 1.00 | 23.91 | O |
| ATOM | 2307 | CB | THR | A | 615 | 6.569 | 34.221 | 78.649 | 1.00 | 24.22 | C |
| ATOM | 2308 | OG1 | THR | A | 615 | 5.377 | 33.898 | 77.928 | 1.00 | 23.99 | O |
| ATOM | 2309 | CG2 | THR | A | 615 | 6.213 | 34.997 | 79.911 | 1.00 | 24.24 | C |
| ATOM | 2310 | N | TRP | A | 616 | 8.923 | 33.276 | 76.842 | 1.00 | 23.60 | N |
| ATOM | 2311 | CA | TRP | A | 616 | 9.427 | 32.368 | 75.828 | 1.00 | 22.94 | C |
| ATOM | 2312 | C | TRP | A | 616 | 9.413 | 30.945 | 76.355 | 1.00 | 22.75 | C |
| ATOM | 2313 | O | TRP | A | 616 | 9.528 | 30.719 | 77.562 | 1.00 | 22.77 | O |
| ATOM | 2314 | CB | TRP | A | 616 | 10.829 | 32.795 | 75.379 | 1.00 | 22.82 | C |
| ATOM | 2315 | CG | TRP | A | 616 | 10.823 | 34.167 | 74.785 | 1.00 | 22.48 | C |
| ATOM | 2316 | CD1 | TRP | A | 616 | 10.945 | 35.354 | 75.455 | 1.00 | 22.44 | C |
| ATOM | 2317 | CD2 | TRP | A | 616 | 10.641 | 34.501 | 73.407 | 1.00 | 22.15 | C |
| ATOM | 2318 | NE1 | TRP | A | 616 | 10.864 | 36.407 | 74.576 | 1.00 | 22.32 | N |
| ATOM | 2319 | CE2 | TRP | A | 616 | 10.680 | 35.913 | 73.311 | 1.00 | 22.46 | C |
| ATOM | 2320 | CE3 | TRP | A | 616 | 10.452 | 33.747 | 72.242 | 1.00 | 21.85 | C |
| ATOM | 2321 | CZ2 | TRP | A | 616 | 10.535 | 36.587 | 72.093 | 1.00 | 22.13 | C |
| ATOM | 2322 | CZ3 | TRP | A | 616 | 10.306 | 34.417 | 71.030 | 1.00 | 22.39 | C |
| ATOM | 2323 | CH2 | TRP | A | 616 | 10.350 | 35.825 | 70.967 | 1.00 | 22.39 | C |
| ATOM | 2324 | N | HIS | A | 617 | 9.245 | 29.992 | 75.444 | 1.00 | 22.54 | N |
| ATOM | 2325 | CA | HIS | A | 617 | 9.227 | 28.577 | 75.797 | 1.00 | 22.35 | C |
| ATOM | 2326 | C | HIS | A | 617 | 10.055 | 27.774 | 74.800 | 1.00 | 21.96 | C |
| ATOM | 2327 | O | HIS | A | 617 | 10.162 | 28.140 | 73.628 | 1.00 | 21.60 | O |
| ATOM | 2328 | CB | HIS | A | 617 | 7.790 | 28.046 | 75.832 | 1.00 | 22.57 | C |
| ATOM | 2329 | CG | HIS | A | 617 | 6.866 | 28.856 | 76.685 | 1.00 | 23.43 | C |
| ATOM | 2330 | ND1 | HIS | A | 617 | 6.673 | 28.600 | 78.027 | 1.00 | 24.70 | N |
| ATOM | 2331 | CD2 | HIS | A | 617 | 6.089 | 29.926 | 76.391 | 1.00 | 23.60 | C |
| ATOM | 2332 | CE1 | HIS | A | 617 | 5.812 | 29.473 | 78.520 | 1.00 | 24.49 | C |
| ATOM | 2333 | NE2 | HIS | A | 617 | 5.446 | 30.292 | 77.549 | 1.00 | 24.05 | N |
| ATOM | 2334 | N | ALA | A | 618 | 10.639 | 26.681 | 75.278 | 1.00 | 21.57 | N |
| ATOM | 2335 | CA | ALA | A | 618 | 11.393 | 25.787 | 74.428 | 1.00 | 21.25 | C |
| ATOM | 2336 | C | ALA | A | 618 | 10.471 | 24.728 | 73.859 | 1.00 | 21.30 | C |
| ATOM | 2337 | O | ALA | A | 618 | 9.708 | 24.093 | 74.598 | 1.00 | 21.22 | O |
| ATOM | 2338 | CB | ALA | A | 618 | 12.518 | 25.137 | 75.206 | 1.00 | 21.19 | C |
| ATOM | 2339 | N | GLY | A | 619 | 10.539 | 24.544 | 72.542 | 1.00 | 21.14 | N |
| ATOM | 2340 | CA | GLY | A | 619 | 9.961 | 23.367 | 71.905 | 1.00 | 21.04 | C |
| ATOM | 2341 | C | GLY | A | 619 | 10.882 | 22.172 | 72.099 | 1.00 | 20.80 | C |
| ATOM | 2342 | O | GLY | A | 619 | 11.972 | 22.304 | 72.653 | 1.00 | 20.60 | O |
| ATOM | 2343 | N | GLU | A | 620 | 10.434 | 21.003 | 71.654 | 1.00 | 21.09 | N |
| ATOM | 2344 | CA | GLU | A | 620 | 11.260 | 19.785 | 71.652 | 1.00 | 21.16 | C |
| ATOM | 2345 | C | GLU | A | 620 | 12.327 | 19.862 | 70.566 | 1.00 | 20.91 | C |
| ATOM | 2346 | O | GLU | A | 620 | 12.144 | 20.543 | 69.553 | 1.00 | 20.92 | O |
| ATOM | 2347 | CB | GLU | A | 620 | 10.395 | 18.541 | 71.411 | 1.00 | 21.10 | C |
| ATOM | 2348 | CG | GLU | A | 620 | 9.360 | 18.267 | 72.503 | 1.00 | 21.22 | C |
| ATOM | 2349 | CD | GLU | A | 620 | 8.778 | 16.850 | 72.456 | 1.00 | 21.67 | C |
| ATOM | 2350 | OE1 | GLU | A | 620 | 9.110 | 16.058 | 71.538 | 1.00 | 21.86 | O |
| ATOM | 2351 | OE2 | GLU | A | 620 | 7.973 | 16.524 | 73.355 | 1.00 | 23.31 | O |
| ATOM | 2352 | N | ALA | A | 621 | 13.443 | 19.170 | 70.779 | 1.00 | 20.48 | N |
| ATOM | 2353 | CA | ALA | A | 621 | 14.465 | 19.058 | 69.750 | 1.00 | 20.13 | C |
| ATOM | 2354 | C | ALA | A | 621 | 14.002 | 18.071 | 68.691 | 1.00 | 20.09 | C |
| ATOM | 2355 | O | ALA | A | 621 | 13.224 | 17.163 | 68.987 | 1.00 | 20.41 | O |
| ATOM | 2356 | CB | ALA | A | 621 | 15.789 | 18.618 | 70.351 | 1.00 | 19.93 | C |
| ATOM | 2357 | N | VAL | A | 622 | 14.476 | 18.258 | 67.460 | 1.00 | 19.87 | N |
| ATOM | 2358 | CA | VAL | A | 622 | 14.234 | 17.325 | 66.363 | 1.00 | 19.78 | C |
| ATOM | 2359 | C | VAL | A | 622 | 14.849 | 15.973 | 66.693 | 1.00 | 20.24 | C |
| ATOM | 2360 | O | VAL | A | 622 | 14.348 | 14.924 | 66.272 | 1.00 | 20.06 | O |
| ATOM | 2361 | CB | VAL | A | 622 | 14.832 | 17.852 | 65.032 | 1.00 | 19.99 | C |
| ATOM | 2362 | CG1 | VAL | A | 622 | 14.748 | 16.794 | 63.930 | 1.00 | 18.57 | C |
| ATOM | 2363 | CG2 | VAL | A | 622 | 14.136 | 19.154 | 64.605 | 1.00 | 19.53 | C |
| ATOM | 2364 | N | ASN | A | 623 | 15.936 | 16.012 | 67.460 | 1.00 | 20.61 | N |
| ATOM | 2365 | CA | ASN | A | 623 | 16.625 | 14.807 | 67.893 | 1.00 | 21.08 | C |
| ATOM | 2366 | C | ASN | A | 623 | 16.016 | 14.100 | 69.116 | 1.00 | 21.43 | C |
| ATOM | 2367 | O | ASN | A | 623 | 16.483 | 13.018 | 69.493 | 1.00 | 21.72 | O |
| ATOM | 2368 | CB | ASN | A | 623 | 18.100 | 15.114 | 68.142 | 1.00 | 21.11 | C |
| ATOM | 2369 | CG | ASN | A | 623 | 18.882 | 15.292 | 66.862 | 1.00 | 21.28 | C |
| ATOM | 2370 | OD1 | ASN | A | 623 | 18.389 | 15.003 | 65.773 | 1.00 | 21.42 | O |
| ATOM | 2371 | ND2 | ASN | A | 623 | 20.118 | 15.765 | 66.987 | 1.00 | 21.57 | N |
| ATOM | 2372 | N | ASP | A | 624 | 14.990 | 14.693 | 69.728 | 1.00 | 21.66 | N |
| ATOM | 2373 | CA | ASP | A | 624 | 14.339 | 14.083 | 70.902 | 1.00 | 22.42 | C |
| ATOM | 2374 | C | ASP | A | 624 | 13.421 | 12.933 | 70.513 | 1.00 | 23.01 | C |
| ATOM | 2375 | O | ASP | A | 624 | 12.462 | 13.117 | 69.755 | 1.00 | 23.13 | O |
| ATOM | 2376 | CB | ASP | A | 624 | 13.577 | 15.126 | 71.737 | 1.00 | 22.11 | C |
| ATOM | 2377 | CG | ASP | A | 624 | 14.508 | 16.097 | 72.456 | 1.00 | 21.59 | C |
| ATOM | 2378 | OD1 | ASP | A | 624 | 15.707 | 15.787 | 72.603 | 1.00 | 20.84 | O |
| ATOM | 2379 | OD2 | ASP | A | 624 | 14.046 | 17.178 | 72.874 | 1.00 | 21.45 | O |
| ATOM | 2380 | N | ASN | A | 625 | 13.741 | 11.754 | 71.043 | 1.00 | 23.78 | N |
| ATOM | 2381 | CA | ASN | A | 625 | 13.041 | 10.489 | 70.765 | 1.00 | 24.64 | C |
| ATOM | 2382 | C | ASN | A | 625 | 12.999 | 10.130 | 69.280 | 1.00 | 24.87 | C |
| ATOM | 2383 | O | ASN | A | 625 | 12.008 | 9.607 | 68.769 | 1.00 | 25.24 | O |
| ATOM | 2384 | CB | ASN | A | 625 | 11.644 | 10.457 | 71.397 | 1.00 | 24.82 | C |
| ATOM | 2385 | CG | ASN | A | 625 | 11.149 | 9.040 | 71.651 | 1.00 | 26.05 | C |
| ATOM | 2386 | OD1 | ASN | A | 625 | 11.934 | 8.126 | 71.942 | 1.00 | 27.20 | O |
| ATOM | 2387 | ND2 | ASN | A | 625 | 9.838 | 8.851 | 71.549 | 1.00 | 27.88 | N |
| ATOM | 2388 | N | ARG | A | 626 | 14.104 | 10.420 | 68.605 | 1.00 | 25.07 | N |
| ATOM | 2389 | CA | ARG | A | 626 | 14.257 | 10.177 | 67.183 | 1.00 | 25.11 | C |
| ATOM | 2390 | C | ARG | A | 626 | 15.064 | 8.904 | 66.962 | 1.00 | 25.29 | C |
| ATOM | 2391 | O | ARG | A | 626 | 15.986 | 8.599 | 67.728 | 1.00 | 25.20 | O |
| ATOM | 2392 | CB | ARG | A | 626 | 14.975 | 11.370 | 66.561 | 1.00 | 25.12 | C |
| ATOM | 2393 | CG | ARG | A | 626 | 15.282 | 11.249 | 65.098 | 1.00 | 24.94 | C |
| ATOM | 2394 | CD | ARG | A | 626 | 16.393 | 12.201 | 64.754 | 1.00 | 25.44 | C |
| ATOM | 2395 | NE | ARG | A | 626 | 16.996 | 11.889 | 63.465 | 1.00 | 25.40 | N |
| ATOM | 2396 | CZ | ARG | A | 626 | 18.180 | 12.334 | 63.070 | 1.00 | 25.64 | C |
| ATOM | 2397 | NH1 | ARG | A | 626 | 18.906 | 13.113 | 63.868 | 1.00 | 25.16 | N |
| ATOM | 2398 | NH2 | ARG | A | 626 | 18.638 | 12.005 | 61.870 | 1.00 | 26.15 | N |
| ATOM | 2399 | N | GLN | A | 627 | 14.729 | 8.163 | 65.911 | 1.00 | 25.46 | N |
| ATOM | 2400 | CA | GLN | A | 627 | 15.436 | 6.926 | 65.627 | 1.00 | 25.58 | C |
| ATOM | 2401 | C | GLN | A | 627 | 16.548 | 7.091 | 64.595 | 1.00 | 25.65 | C |
| ATOM | 2402 | O | GLN | A | 627 | 16.310 | 7.481 | 63.453 | 1.00 | 25.58 | O |
| ATOM | 2403 | CB | GLN | A | 627 | 14.469 | 5.821 | 65.220 | 1.00 | 25.63 | C |
| ATOM | 2404 | CG | GLN | A | 627 | 14.942 | 4.452 | 65.670 | 1.00 | 26.15 | C |
| ATOM | 2405 | CD | GLN | A | 627 | 13.865 | 3.396 | 65.561 | 1.00 | 26.36 | C |
| ATOM | 2406 | OE1 | GLN | A | 627 | 13.144 | 3.125 | 66.525 | 1.00 | 26.87 | O |
| ATOM | 2407 | NE2 | GLN | A | 627 | 13.741 | 2.799 | 64.381 | 1.00 | 25.79 | N |
| ATOM | 2408 | N | VAL | A | 628 | 17.769 | 6.802 | 65.036 | 1.00 | 25.97 | N |
| ATOM | 2409 | CA | VAL | A | 628 | 18.946 | 6.768 | 64.180 | 1.00 | 25.97 | C |
| ATOM | 2410 | C | VAL | A | 628 | 19.500 | 5.348 | 64.217 | 1.00 | 26.20 | C |
| ATOM | 2411 | O | VAL | A | 628 | 19.906 | 4.871 | 65.274 | 1.00 | 26.15 | O |
| ATOM | 2412 | CB | VAL | A | 628 | 20.031 | 7.763 | 64.674 | 1.00 | 25.89 | C |
| ATOM | 2413 | CG1 | VAL | A | 628 | 21.336 | 7.597 | 63.889 | 1.00 | 25.59 | C |
| ATOM | 2414 | CG2 | VAL | A | 628 | 19.528 | 9.190 | 64.581 | 1.00 | 25.81 | C |
| ATOM | 2415 | N | ASP | A | 629 | 19.503 | 4.677 | 63.066 | 1.00 | 26.67 | N |
| ATOM | 2416 | CA | ASP | A | 629 | 20.043 | 3.315 | 62.942 | 1.00 | 27.40 | C |
| ATOM | 2417 | C | ASP | A | 629 | 19.442 | 2.323 | 63.944 | 1.00 | 27.57 | C |
| ATOM | 2418 | O | ASP | A | 629 | 20.165 | 1.508 | 64.533 | 1.00 | 27.35 | O |
| ATOM | 2419 | CB | ASP | A | 629 | 21.577 | 3.322 | 63.059 | 1.00 | 27.72 | C |
| ATOM | 2420 | CG | ASP | A | 629 | 22.249 | 4.124 | 61.955 | 1.00 | 28.52 | C |
| ATOM | 2421 | OD1 | ASP | A | 629 | 21.836 | 3.999 | 60.781 | 1.00 | 28.82 | O |
| ATOM | 2422 | OD2 | ASP | A | 629 | 23.194 | 4.882 | 62.265 | 1.00 | 29.81 | O |
| ATOM | 2423 | N | GLY | A | 630 | 18.124 | 2.409 | 64.137 | 1.00 | 27.86 | N |
| ATOM | 2424 | CA | GLY | A | 630 | 17.400 | 1.514 | 65.042 | 1.00 | 28.19 | C |
| ATOM | 2425 | C | GLY | A | 630 | 17.523 | 1.836 | 66.522 | 1.00 | 28.60 | C |
| ATOM | 2426 | O | GLY | A | 630 | 16.931 | 1.153 | 67.352 | 1.00 | 28.89 | O |
| ATOM | 2427 | N | GLN | A | 631 | 18.289 | 2.869 | 66.863 | 1.00 | 28.79 | N |
| ATOM | 2428 | CA | GLN | A | 631 | 18.447 | 3.282 | 68.261 | 1.00 | 29.24 | C |
| ATOM | 2429 | C | GLN | A | 631 | 17.936 | 4.700 | 68.471 | 1.00 | 28.48 | C |
| ATOM | 2430 | O | GLN | A | 631 | 18.369 | 5.624 | 67.785 | 1.00 | 28.59 | O |
| ATOM | 2431 | CB | GLN | A | 631 | 19.913 | 3.182 | 68.693 | 1.00 | 29.15 | C |
| ATOM | 2432 | CG | GLN | A | 631 | 20.411 | 1.746 | 68.908 | 1.00 | 30.64 | C |
| ATOM | 2433 | CD | GLN | A | 631 | 21.921 | 1.660 | 69.162 | 1.00 | 31.20 | C |
| ATOM | 2434 | OE1 | GLN | A | 631 | 22.660 | 2.646 | 69.015 | 1.00 | 32.91 | O |
| ATOM | 2435 | NE2 | GLN | A | 631 | 22.382 | 0.471 | 69.544 | 1.00 | 34.05 | N |
| ATOM | 2436 | N | LYS | A | 632 | 17.013 | 4.870 | 69.415 | 1.00 | 27.81 | N |
| ATOM | 2437 | CA | LYS | A | 632 | 16.464 | 6.192 | 69.711 | 1.00 | 27.29 | C |
| ATOM | 2438 | C | LYS | A | 632 | 17.482 | 7.063 | 70.435 | 1.00 | 26.44 | C |
| ATOM | 2439 | O | LYS | A | 632 | 18.198 | 6.580 | 71.305 | 1.00 | 26.43 | O |
| ATOM | 2440 | CB | LYS | A | 632 | 15.177 | 6.085 | 70.528 | 1.00 | 27.27 | C |
| ATOM | 2441 | CG | LYS | A | 632 | 14.027 | 5.412 | 69.779 | 1.00 | 28.01 | C |
| ATOM | 2442 | CD | LYS | A | 632 | 12.762 | 5.342 | 70.630 | 1.00 | 28.31 | C |
| ATOM | 2443 | CE | LYS | A | 632 | 11.787 | 4.286 | 70.115 | 1.00 | 30.01 | C |
| ATOM | 2444 | NZ | LYS | A | 632 | 11.333 | 4.548 | 68.714 | 1.00 | 31.65 | N |
| ATOM | 2445 | N | ILE | A | 633 | 17.554 | 8.336 | 70.047 | 1.00 | 25.39 | N |
| ATOM | 2446 | CA | ILE | A | 633 | 18.409 | 9.312 | 70.717 | 1.00 | 24.44 | C |
| ATOM | 2447 | C | ILE | A | 633 | 17.588 | 10.413 | 71.395 | 1.00 | 23.93 | C |
| ATOM | 2448 | O | ILE | A | 633 | 16.364 | 10.484 | 71.240 | 1.00 | 23.85 | O |
| ATOM | 2449 | CB | ILE | A | 633 | 19.454 | 9.961 | 69.759 | 1.00 | 24.42 | C |
| ATOM | 2450 | CG1 | ILE | A | 633 | 18.772 | 10.618 | 68.553 | 1.00 | 24.76 | C |
| ATOM | 2451 | CG2 | ILE | A | 633 | 20.498 | 8.944 | 69.328 | 1.00 | 25.00 | C |
| ATOM | 2452 | CD1 | ILE | A | 633 | 19.696 | 11.464 | 67.679 | 1.00 | 24.33 | C |
| ATOM | 2453 | N | HIS | A | 634 | 18.280 | 11.254 | 72.157 | 1.00 | 22.96 | N |
| ATOM | 2454 | CA | HIS | A | 634 | 17.702 | 12.428 | 72.772 | 1.00 | 22.28 | C |
| ATOM | 2455 | C | HIS | A | 634 | 18.799 | 13.468 | 72.775 | 1.00 | 22.05 | C |
| ATOM | 2456 | O | HIS | A | 634 | 19.974 | 13.119 | 72.896 | 1.00 | 22.07 | O |
| ATOM | 2457 | CB | HIS | A | 634 | 17.272 | 12.120 | 74.205 | 1.00 | 22.30 | C |
| ATOM | 2458 | CG | HIS | A | 634 | 16.469 | 13.208 | 74.844 | 1.00 | 21.71 | C |
| ATOM | 2459 | ND1 | HIS | A | 634 | 17.044 | 14.240 | 75.557 | 1.00 | 21.42 | N |
| ATOM | 2460 | CD2 | HIS | A | 634 | 15.134 | 13.427 | 74.879 | 1.00 | 21.28 | C |
| ATOM | 2461 | CE1 | HIS | A | 634 | 16.099 | 15.047 | 76.002 | 1.00 | 20.31 | C |
| ATOM | 2462 | NE2 | HIS | A | 634 | 14.930 | 14.576 | 75.605 | 1.00 | 21.37 | N |
| ATOM | 2463 | N | SER | A | 635 | 18.427 | 14.739 | 72.646 | 1.00 | 21.64 | N |
| ATOM | 2464 | CA | SER | A | 635 | 19.420 | 15.814 | 72.545 | 1.00 | 21.40 | C |
| ATOM | 2465 | C | SER | A | 635 | 20.261 | 15.999 | 73.819 | 1.00 | 21.25 | C |
| ATOM | 2466 | O | SER | A | 635 | 21.356 | 16.564 | 73.762 | 1.00 | 21.25 | O |
| ATOM | 2467 | CB | SER | A | 635 | 18.761 | 17.131 | 72.121 | 1.00 | 21.27 | C |
| ATOM | 2468 | OG | SER | A | 635 | 17.712 | 17.497 | 72.994 | 1.00 | 21.38 | O |
| ATOM | 2469 | N | SER | A | 636 | 19.753 | 15.508 | 74.950 | 1.00 | 20.81 | N |
| ATOM | 2470 | CA | SER | A | 636 | 20.449 | 15.610 | 76.232 | 1.00 | 20.67 | C |
| ATOM | 2471 | C | SER | A | 636 | 21.491 | 14.519 | 76.465 | 1.00 | 20.76 | C |
| ATOM | 2472 | O | SER | A | 636 | 22.358 | 14.673 | 77.319 | 1.00 | 20.42 | O |
| ATOM | 2473 | CB | SER | A | 636 | 19.447 | 15.617 | 77.392 | 1.00 | 20.58 | C |
| ATOM | 2474 | OG | SER | A | 636 | 18.786 | 14.371 | 77.522 | 1.00 | 20.58 | O |
| ATOM | 2475 | N | THR | A | 637 | 21.404 | 13.426 | 75.705 | 1.00 | 21.05 | N |
| ATOM | 2476 | CA | THR | A | 637 | 22.253 | 12.252 | 75.934 | 1.00 | 21.37 | C |
| ATOM | 2477 | C | THR | A | 637 | 23.001 | 11.766 | 74.685 | 1.00 | 21.52 | C |
| ATOM | 2478 | O | THR | A | 637 | 23.775 | 10.808 | 74.752 | 1.00 | 21.36 | O |
| ATOM | 2479 | CB | THR | A | 637 | 21.428 | 11.068 | 76.516 | 1.00 | 21.66 | C |
| ATOM | 2480 | OG1 | THR | A | 637 | 20.285 | 10.812 | 75.683 | 1.00 | 21.94 | O |
| ATOM | 2481 | CG2 | THR | A | 637 | 20.956 | 11.370 | 77.949 | 1.00 | 21.43 | C |
| ATOM | 2482 | N | MET | A | 638 | 22.776 | 12.427 | 73.551 | 1.00 | 21.74 | N |
| ATOM | 2483 | CA | MET | A | 638 | 23.315 | 11.947 | 72.280 | 1.00 | 21.69 | C |
| ATOM | 2484 | C | MET | A | 638 | 24.821 | 12.166 | 72.108 | 1.00 | 21.73 | C |
| ATOM | 2485 | O | MET | A | 638 | 25.398 | 13.115 | 72.656 | 1.00 | 21.51 | O |
| ATOM | 2486 | CB | MET | A | 638 | 22.527 | 12.514 | 71.091 | 1.00 | 21.79 | C |
| ATOM | 2487 | CG | MET | A | 638 | 22.860 | 13.945 | 70.715 | 1.00 | 21.58 | C |
| ATOM | 2488 | SD | MET | A | 638 | 21.951 | 14.519 | 69.274 | 1.00 | 21.85 | S |
| ATOM | 2489 | CE | MET | A | 638 | 22.867 | 13.792 | 67.915 | 1.00 | 22.09 | C |
| ATOM | 2490 | N | ASN | A | 639 | 25.438 | 11.261 | 71.348 | 1.00 | 21.83 | N |
| ATOM | 2491 | CA | ASN | A | 639 | 26.846 | 11.339 | 70.969 | 1.00 | 21.80 | C |
| ATOM | 2492 | C | ASN | A | 639 | 27.021 | 10.677 | 69.610 | 1.00 | 22.19 | C |
| ATOM | 2493 | O | ASN | A | 639 | 27.470 | 9.535 | 69.497 | 1.00 | 22.24 | O |
| ATOM | 2494 | CB | ASN | A | 639 | 27.757 | 10.694 | 72.022 | 1.00 | 21.36 | C |
| ATOM | 2495 | CG | ASN | A | 639 | 29.242 | 10.811 | 71.665 | 1.00 | 20.90 | C |
| ATOM | 2496 | OD1 | ASN | A | 639 | 29.645 | 11.683 | 70.891 | 1.00 | 19.94 | O |
| ATOM | 2497 | ND2 | ASN | A | 639 | 30.055 | 9.925 | 72.224 | 1.00 | 19.34 | N |
| ATOM | 2498 | N | ASN | A | 640 | 26.638 | 11.417 | 68.579 | 1.00 | 22.77 | N |
| ATOM | 2499 | CA | ASN | A | 640 | 26.600 | 10.920 | 67.219 | 1.00 | 23.26 | C |
| ATOM | 2500 | C | ASN | A | 640 | 26.814 | 12.109 | 66.305 | 1.00 | 23.96 | C |
| ATOM | 2501 | O | ASN | A | 640 | 25.931 | 12.965 | 66.171 | 1.00 | 23.89 | O |
| ATOM | 2502 | CB | ASN | A | 640 | 25.246 | 10.257 | 66.940 | 1.00 | 23.03 | C |
| ATOM | 2503 | CG | ASN | A | 640 | 25.149 | 9.668 | 65.542 | 1.00 | 22.82 | C |
| ATOM | 2504 | OD1 | ASN | A | 640 | 25.458 | 10.323 | 64.541 | 1.00 | 22.96 | O |
| ATOM | 2505 | ND2 | ASN | A | 640 | 24.702 | 8.423 | 65.468 | 1.00 | 22.01 | N |
| ATOM | 2506 | N | ARG | A | 641 | 27.995 | 12.154 | 65.689 | 1.00 | 24.62 | N |
| ATOM | 2507 | CA | ARG | A | 641 | 28.410 | 13.276 | 64.855 | 1.00 | 25.23 | C |
| ATOM | 2508 | C | ARG | A | 641 | 27.431 | 13.531 | 63.711 | 1.00 | 25.17 | C |
| ATOM | 2509 | O | ARG | A | 641 | 27.003 | 14.675 | 63.488 | 1.00 | 25.40 | O |
| ATOM | 2510 | CB | ARG | A | 641 | 29.816 | 13.038 | 64.303 | 1.00 | 25.26 | C |
| ATOM | 2511 | CG | ARG | A | 641 | 30.494 | 14.304 | 63.823 | 1.00 | 26.40 | C |
| ATOM | 2512 | CD | ARG | A | 641 | 31.906 | 14.066 | 63.300 | 1.00 | 26.66 | C |
| ATOM | 2513 | NE | ARG | A | 641 | 32.303 | 15.169 | 62.423 | 1.00 | 29.71 | N |
| ATOM | 2514 | CZ | ARG | A | 641 | 32.069 | 15.208 | 61.110 | 1.00 | 30.42 | C |
| ATOM | 2515 | NH1 | ARG | A | 641 | 31.456 | 14.191 | 60.506 | 1.00 | 29.21 | N |
| ATOM | 2516 | NH2 | ARG | A | 641 | 32.450 | 16.266 | 60.396 | 1.00 | 30.64 | N |
| ATOM | 2517 | N | ARG | A | 642 | 27.067 | 12.456 | 63.016 | 1.00 | 24.80 | N |
| ATOM | 2518 | CA | ARG | A | 642 | 26.251 | 12.528 | 61.810 | 1.00 | 24.72 | C |
| ATOM | 2519 | C | ARG | A | 642 | 24.798 | 12.958 | 62.068 | 1.00 | 24.18 | C |
| ATOM | 2520 | O | ARG | A | 642 | 24.145 | 13.521 | 61.183 | 1.00 | 24.00 | O |
| ATOM | 2521 | CB | ARG | A | 642 | 26.277 | 11.170 | 61.100 | 1.00 | 25.06 | C |
| ATOM | 2522 | CG | ARG | A | 642 | 25.582 | 11.150 | 59.754 | 1.00 | 26.28 | C |
| ATOM | 2523 | CD | ARG | A | 642 | 25.885 | 9.875 | 58.986 | 1.00 | 27.98 | C |
| ATOM | 2524 | NE | ARG | A | 642 | 25.277 | 9.892 | 57.656 | 1.00 | 29.07 | N |
| ATOM | 2525 | CZ | ARG | A | 642 | 25.811 | 10.483 | 56.589 | 1.00 | 29.96 | C |
| ATOM | 2526 | NH1 | ARG | A | 642 | 26.977 | 11.122 | 56.676 | 1.00 | 30.08 | N |
| ATOM | 2527 | NH2 | ARG | A | 642 | 25.178 | 10.434 | 55.425 | 1.00 | 30.47 | N |
| ATOM | 2528 | N | ALA | A | 643 | 24.299 | 12.691 | 63.274 | 1.00 | 23.32 | N |
| ATOM | 2529 | CA | ALA | A | 643 | 22.886 | 12.900 | 63.576 | 1.00 | 22.45 | C |
| ATOM | 2530 | C | ALA | A | 643 | 22.599 | 14.305 | 64.108 | 1.00 | 21.96 | C |
| ATOM | 2531 | O | ALA | A | 643 | 21.452 | 14.647 | 64.405 | 1.00 | 22.03 | O |
| ATOM | 2532 | CB | ALA | A | 643 | 22.379 | 11.832 | 64.537 | 1.00 | 22.39 | C |
| ATOM | 2533 | N | GLN | A | 644 | 23.643 | 15.117 | 64.208 | 1.00 | 21.07 | N |
| ATOM | 2534 | CA | GLN | A | 644 | 23.504 | 16.490 | 64.651 | 1.00 | 20.46 | C |
| ATOM | 2535 | C | GLN | A | 644 | 22.749 | 17.349 | 63.649 | 1.00 | 20.36 | C |
| ATOM | 2536 | O | GLN | A | 644 | 22.744 | 17.081 | 62.446 | 1.00 | 20.22 | O |
| ATOM | 2537 | CB | GLN | A | 644 | 24.873 | 17.118 | 64.871 | 1.00 | 20.43 | C |
| ATOM | 2538 | CG | GLN | A | 644 | 25.622 | 16.628 | 66.086 | 1.00 | 20.25 | C |
| ATOM | 2539 | CD | GLN | A | 644 | 27.013 | 17.197 | 66.131 | 1.00 | 19.57 | C |
| ATOM | 2540 | OE1 | GLN | A | 644 | 27.301 | 18.088 | 66.918 | 1.00 | 18.79 | O |
| ATOM | 2541 | NE2 | GLN | A | 644 | 27.881 | 16.704 | 65.259 | 1.00 | 20.47 | N |
| ATOM | 2542 | N | ASN | A | 645 | 22.115 | 18.388 | 64.170 | 1.00 | 20.08 | N |
| ATOM | 2543 | CA | ASN | A | 645 | 21.549 | 19.449 | 63.368 | 1.00 | 19.92 | C |
| ATOM | 2544 | C | ASN | A | 645 | 21.682 | 20.715 | 64.183 | 1.00 | 20.00 | C |
| ATOM | 2545 | O | ASN | A | 645 | 21.425 | 20.713 | 65.382 | 1.00 | 20.06 | O |
| ATOM | 2546 | CB | ASN | A | 645 | 20.098 | 19.164 | 62.975 | 1.00 | 19.96 | C |
| ATOM | 2547 | CG | ASN | A | 645 | 19.363 | 18.310 | 63.989 | 1.00 | 19.74 | C |
| ATOM | 2548 | OD1 | ASN | A | 645 | 19.009 | 18.774 | 65.071 | 1.00 | 21.27 | O |
| ATOM | 2549 | ND2 | ASN | A | 645 | 19.105 | 17.061 | 63.630 | 1.00 | 18.29 | N |
| ATOM | 2550 | N | THR | A | 646 | 22.106 | 21.795 | 63.541 | 1.00 | 20.14 | N |
| ATOM | 2551 | CA | THR | A | 646 | 22.610 | 22.942 | 64.288 | 1.00 | 20.17 | C |
| ATOM | 2552 | C | THR | A | 646 | 21.773 | 24.198 | 64.043 | 1.00 | 20.35 | C |
| ATOM | 2553 | O | THR | A | 646 | 20.577 | 24.229 | 64.359 | 1.00 | 20.31 | O |
| ATOM | 2554 | CB | THR | A | 646 | 24.112 | 23.175 | 63.990 | 1.00 | 20.10 | C |
| ATOM | 2555 | OG1 | THR | A | 646 | 24.264 | 23.764 | 62.694 | 1.00 | 19.88 | O |
| ATOM | 2556 | CG2 | THR | A | 646 | 24.869 | 21.856 | 64.020 | 1.00 | 19.40 | C |
| ATOM | 2557 | N | GLU | A | 647 | 22.411 | 25.231 | 63.501 | 1.00 | 20.59 | N |
| ATOM | 2558 | CA | GLU | A | 647 | 21.714 | 26.416 | 63.017 | 1.00 | 20.79 | C |
| ATOM | 2559 | C | GLU | A | 647 | 20.654 | 25.957 | 62.037 | 1.00 | 20.46 | C |
| ATOM | 2560 | O | GLU | A | 647 | 20.857 | 24.995 | 61.297 | 1.00 | 20.49 | O |
| ATOM | 2561 | CB | GLU | A | 647 | 22.679 | 27.366 | 62.300 | 1.00 | 20.58 | C |
| ATOM | 2562 | CG | GLU | A | 647 | 23.921 | 27.735 | 63.093 | 1.00 | 20.89 | C |
| ATOM | 2563 | CD | GLU | A | 647 | 24.990 | 28.405 | 62.236 | 1.00 | 21.39 | C |
| ATOM | 2564 | OE1 | GLU | A | 647 | 25.171 | 28.020 | 61.062 | 1.00 | 21.55 | O |
| ATOM | 2565 | OE2 | GLU | A | 647 | 25.659 | 29.327 | 62.743 | 1.00 | 23.40 | O |
| ATOM | 2566 | N | SER | A | 648 | 19.518 | 26.639 | 62.036 | 1.00 | 20.32 | N |
| ATOM | 2567 | CA | SER | A | 648 | 18.436 | 26.256 | 61.154 | 1.00 | 20.16 | C |
| ATOM | 2568 | C | SER | A | 648 | 17.676 | 27.475 | 60.687 | 1.00 | 19.99 | C |
| ATOM | 2569 | O | SER | A | 648 | 17.863 | 28.579 | 61.215 | 1.00 | 19.85 | O |
| ATOM | 2570 | CB | SER | A | 648 | 17.494 | 25.286 | 61.869 | 1.00 | 20.05 | C |
| ATOM | 2571 | OG | SER | A | 648 | 16.943 | 25.901 | 63.018 | 1.00 | 20.75 | O |
| ATOM | 2572 | N | THR | A | 649 | 16.831 | 27.274 | 59.683 | 1.00 | 19.80 | N |
| ATOM | 2573 | CA | THR | A | 649 | 15.919 | 28.315 | 59.248 | 1.00 | 20.07 | C |
| ATOM | 2574 | C | THR | A | 649 | 14.485 | 27.778 | 59.171 | 1.00 | 20.30 | C |
| ATOM | 2575 | O | THR | A | 649 | 14.249 | 26.653 | 58.713 | 1.00 | 20.15 | O |
| ATOM | 2576 | CB | THR | A | 649 | 16.391 | 28.993 | 57.940 | 1.00 | 20.08 | C |
| ATOM | 2577 | OG1 | THR | A | 649 | 15.546 | 30.112 | 57.650 | 1.00 | 20.03 | O |
| ATOM | 2578 | CG2 | THR | A | 649 | 16.393 | 28.017 | 56.761 | 1.00 | 20.04 | C |
| ATOM | 2579 | N | VAL | A | 650 | 13.540 | 28.587 | 59.642 | 1.00 | 20.57 | N |
| ATOM | 2580 | CA | VAL | A | 650 | 12.167 | 28.133 | 59.879 | 1.00 | 20.87 | C |
| ATOM | 2581 | C | VAL | A | 650 | 11.173 | 28.761 | 58.891 | 1.00 | 21.10 | C |
| ATOM | 2582 | O | VAL | A | 650 | 11.257 | 29.958 | 58.611 | 1.00 | 21.07 | O |
| ATOM | 2583 | CB | VAL | A | 650 | 11.754 | 28.393 | 61.370 | 1.00 | 20.97 | C |
| ATOM | 2584 | CG1 | VAL | A | 650 | 11.632 | 29.900 | 61.674 | 1.00 | 20.68 | C |
| ATOM | 2585 | CG2 | VAL | A | 650 | 10.483 | 27.646 | 61.733 | 1.00 | 20.39 | C |
| ATOM | 2586 | N | VAL | A | 651 | 10.262 | 27.944 | 58.347 | 1.00 | 21.38 | N |
| ATOM | 2587 | CA | VAL | A | 651 | 9.205 | 28.424 | 57.435 | 1.00 | 21.76 | C |
| ATOM | 2588 | C | VAL | A | 651 | 7.855 | 27.729 | 57.681 | 1.00 | 21.93 | C |
| ATOM | 2589 | O | VAL | A | 651 | 7.808 | 26.512 | 57.909 | 1.00 | 21.81 | O |
| ATOM | 2590 | CB | VAL | A | 651 | 9.579 | 28.274 | 55.914 | 1.00 | 21.91 | C |
| ATOM | 2591 | CG1 | VAL | A | 651 | 10.995 | 28.751 | 55.615 | 1.00 | 21.91 | C |
| ATOM | 2592 | CG2 | VAL | A | 651 | 9.419 | 26.852 | 55.443 | 1.00 | 22.76 | C |
| ATOM | 2593 | N | GLN | A | 652 | 6.766 | 28.497 | 57.628 | 1.00 | 21.99 | N |
| ATOM | 2594 | CA | GLN | A | 652 | 5.422 | 27.913 | 57.671 | 1.00 | 22.26 | C |
| ATOM | 2595 | C | GLN | A | 652 | 4.812 | 27.768 | 56.277 | 1.00 | 22.37 | C |
| ATOM | 2596 | O | GLN | A | 652 | 4.913 | 28.672 | 55.445 | 1.00 | 22.33 | O |
| ATOM | 2597 | CB | GLN | A | 652 | 4.465 | 28.706 | 58.582 | 1.00 | 22.32 | C |
| ATOM | 2598 | CG | GLN | A | 652 | 3.155 | 27.940 | 58.861 | 1.00 | 22.11 | C |
| ATOM | 2599 | CD | GLN | A | 652 | 2.193 | 28.656 | 59.796 | 1.00 | 22.16 | C |
| ATOM | 2600 | OE1 | GLN | A | 652 | 2.003 | 29.869 | 59.716 | 1.00 | 21.85 | O |
| ATOM | 2601 | NE2 | GLN | A | 652 | 1.562 | 27.893 | 60.679 | 1.00 | 21.83 | N |
| ATOM | 2602 | N | LEU | A | 653 | 4.178 | 26.621 | 56.039 | 1.00 | 22.74 | N |
| ATOM | 2603 | CA | LEU | A | 653 | 3.424 | 26.376 | 54.806 | 1.00 | 23.06 | C |
| ATOM | 2604 | C | LEU | A | 653 | 2.018 | 26.960 | 54.896 | 1.00 | 22.97 | C |
| ATOM | 2605 | O | LEU | A | 653 | 1.538 | 27.291 | 55.987 | 1.00 | 23.10 | O |
| ATOM | 2606 | CB | LEU | A | 653 | 3.332 | 24.871 | 54.512 | 1.00 | 22.88 | C |
| ATOM | 2607 | CG | LEU | A | 653 | 4.600 | 24.073 | 54.208 | 1.00 | 23.60 | C |
| ATOM | 2608 | CD1 | LEU | A | 653 | 4.231 | 22.667 | 53.769 | 1.00 | 23.89 | C |
| ATOM | 2609 | CD2 | LEU | A | 653 | 5.464 | 24.747 | 53.142 | 1.00 | 24.70 | C |
| ATOM | 2610 | N | ASN | A | 654 | 1.358 | 27.076 | 53.746 | 1.00 | 23.03 | N |
| ATOM | 2611 | CA | ASN | A | 654 | −0.050 | 27.480 | 53.699 | 1.00 | 23.03 | C |
| ATOM | 2612 | C | ASN | A | 654 | −1.002 | 26.469 | 54.336 | 1.00 | 22.64 | C |
| ATOM | 2613 | O | ASN | A | 654 | −2.132 | 26.816 | 54.678 | 1.00 | 22.55 | O |
| ATOM | 2614 | CB | ASN | A | 654 | −0.482 | 27.802 | 52.262 | 1.00 | 23.23 | C |
| ATOM | 2615 | CG | ASN | A | 654 | 0.090 | 29.114 | 51.772 | 1.00 | 23.57 | C |
| ATOM | 2616 | OD1 | ASN | A | 654 | 0.359 | 30.017 | 52.563 | 1.00 | 23.90 | O |
| ATOM | 2617 | ND2 | ASN | A | 654 | 0.288 | 29.226 | 50.464 | 1.00 | 24.45 | N |
| ATOM | 2618 | N | ASN | A | 655 | −0.533 | 25.235 | 54.508 | 1.00 | 22.36 | N |
| ATOM | 2619 | CA | ASN | A | 655 | −1.314 | 24.196 | 55.171 | 1.00 | 22.27 | C |
| ATOM | 2620 | C | ASN | A | 655 | −1.059 | 24.077 | 56.681 | 1.00 | 22.38 | C |
| ATOM | 2621 | O | ASN | A | 655 | −1.567 | 23.163 | 57.332 | 1.00 | 22.03 | O |
| ATOM | 2622 | CB | ASN | A | 655 | −1.146 | 22.846 | 54.461 | 1.00 | 22.38 | C |
| ATOM | 2623 | CG | ASN | A | 655 | 0.224 | 22.215 | 54.674 | 1.00 | 23.37 | C |
| ATOM | 2624 | OD1 | ASN | A | 655 | 1.078 | 22.737 | 55.390 | 1.00 | 24.64 | O |
| ATOM | 2625 | ND2 | ASN | A | 655 | 0.428 | 21.066 | 54.051 | 1.00 | 24.33 | N |
| ATOM | 2626 | N | GLY | A | 656 | −0.271 | 25.007 | 57.226 | 1.00 | 22.54 | N |
| ATOM | 2627 | CA | GLY | A | 656 | −0.045 | 25.088 | 58.669 | 1.00 | 22.40 | C |
| ATOM | 2628 | C | GLY | A | 656 | 1.198 | 24.373 | 59.163 | 1.00 | 22.46 | C |
| ATOM | 2629 | O | GLY | A | 656 | 1.615 | 24.552 | 60.314 | 1.00 | 22.39 | O |
| ATOM | 2630 | N | ASP | A | 657 | 1.794 | 23.558 | 58.301 | 1.00 | 22.33 | N |
| ATOM | 2631 | CA | ASP | A | 657 | 3.000 | 22.841 | 58.674 | 1.00 | 22.26 | C |
| ATOM | 2632 | C | ASP | A | 657 | 4.168 | 23.805 | 58.783 | 1.00 | 22.25 | C |
| ATOM | 2633 | O | ASP | A | 657 | 4.215 | 24.840 | 58.097 | 1.00 | 22.00 | O |
| ATOM | 2634 | CB | ASP | A | 657 | 3.318 | 21.728 | 57.675 | 1.00 | 22.36 | C |
| ATOM | 2635 | CG | ASP | A | 657 | 2.476 | 20.480 | 57.898 | 1.00 | 23.55 | C |
| ATOM | 2636 | OD1 | ASP | A | 657 | 1.557 | 20.510 | 58.753 | 1.00 | 24.51 | O |
| ATOM | 2637 | OD2 | ASP | A | 657 | 2.741 | 19.462 | 57.217 | 1.00 | 23.42 | O |
| ATOM | 2638 | N | VAL | A | 658 | 5.090 | 23.457 | 59.675 | 1.00 | 21.80 | N |
| ATOM | 2639 | CA | VAL | A | 658 | 6.314 | 24.194 | 59.873 | 1.00 | 21.32 | C |
| ATOM | 2640 | C | VAL | A | 658 | 7.455 | 23.340 | 59.363 | 1.00 | 21.06 | C |
| ATOM | 2641 | O | VAL | A | 658 | 7.556 | 22.160 | 59.700 | 1.00 | 21.44 | O |
| ATOM | 2642 | CB | VAL | A | 658 | 6.542 | 24.513 | 61.366 | 1.00 | 21.44 | C |
| ATOM | 2643 | CG1 | VAL | A | 658 | 7.868 | 25.237 | 61.567 | 1.00 | 20.76 | C |
| ATOM | 2644 | CG2 | VAL | A | 658 | 5.387 | 25.336 | 61.916 | 1.00 | 21.18 | C |
| ATOM | 2645 | N | LYS | A | 659 | 8.309 | 23.939 | 58.545 | 1.00 | 20.59 | N |
| ATOM | 2646 | CA | LYS | A | 659 | 9.489 | 23.255 | 58.044 | 1.00 | 20.15 | C |
| ATOM | 2647 | C | LYS | A | 659 | 10.743 | 23.890 | 58.629 | 1.00 | 19.44 | C |
| ATOM | 2648 | O | LYS | A | 659 | 10.875 | 25.116 | 58.668 | 1.00 | 18.90 | O |
| ATOM | 2649 | CB | LYS | A | 659 | 9.525 | 23.277 | 56.510 | 1.00 | 20.26 | C |
| ATOM | 2650 | CG | LYS | A | 659 | 8.336 | 22.592 | 55.836 | 1.00 | 21.28 | C |
| ATOM | 2651 | CD | LYS | A | 659 | 8.518 | 21.077 | 55.765 | 1.00 | 23.70 | C |
| ATOM | 2652 | CE | LYS | A | 659 | 7.376 | 20.412 | 55.007 | 1.00 | 24.90 | C |
| ATOM | 2653 | NZ | LYS | A | 659 | 7.380 | 18.931 | 55.217 | 1.00 | 26.29 | N |
| ATOM | 2654 | N | LEU | A | 660 | 11.648 | 23.039 | 59.101 | 1.00 | 19.21 | N |
| ATOM | 2655 | CA | LEU | A | 660 | 12.912 | 23.489 | 59.675 | 1.00 | 18.87 | C |
| ATOM | 2656 | C | LEU | A | 660 | 14.096 | 22.978 | 58.860 | 1.00 | 18.78 | C |
| ATOM | 2657 | O | LEU | A | 660 | 14.420 | 21.791 | 58.873 | 1.00 | 18.71 | O |
| ATOM | 2658 | CB | LEU | A | 660 | 13.021 | 23.071 | 61.140 | 1.00 | 18.45 | C |
| ATOM | 2659 | CG | LEU | A | 660 | 14.061 | 23.774 | 62.015 | 1.00 | 18.35 | C |
| ATOM | 2660 | CD1 | LEU | A | 660 | 13.700 | 25.232 | 62.286 | 1.00 | 16.99 | C |
| ATOM | 2661 | CD2 | LEU | A | 660 | 14.222 | 23.015 | 63.323 | 1.00 | 18.65 | C |
| ATOM | 2662 | N | PHE | A | 661 | 14.727 | 23.894 | 58.138 | 1.00 | 18.91 | N |
| ATOM | 2663 | CA | PHE | A | 661 | 15.892 | 23.577 | 57.334 | 1.00 | 19.18 | C |
| ATOM | 2664 | C | PHE | A | 661 | 17.131 | 23.708 | 58.211 | 1.00 | 19.48 | C |
| ATOM | 2665 | O | PHE | A | 661 | 17.438 | 24.790 | 58.701 | 1.00 | 19.25 | O |
| ATOM | 2666 | CB | PHE | A | 661 | 15.965 | 24.509 | 56.129 | 1.00 | 19.00 | C |
| ATOM | 2667 | CG | PHE | A | 661 | 14.732 | 24.489 | 55.282 | 1.00 | 19.11 | C |
| ATOM | 2668 | CD1 | PHE | A | 661 | 14.646 | 23.645 | 54.181 | 1.00 | 19.13 | C |
| ATOM | 2669 | CD2 | PHE | A | 661 | 13.644 | 25.303 | 55.591 | 1.00 | 19.18 | C |
| ATOM | 2670 | CE1 | PHE | A | 661 | 13.497 | 23.615 | 53.389 | 1.00 | 19.04 | C |
| ATOM | 2671 | CE2 | PHE | A | 661 | 12.493 | 25.279 | 54.807 | 1.00 | 19.57 | C |
| ATOM | 2672 | CZ | PHE | A | 661 | 12.423 | 24.431 | 53.699 | 1.00 | 19.06 | C |
| ATOM | 2673 | N | MET | A | 662 | 17.831 | 22.594 | 58.402 | 1.00 | 19.92 | N |
| ATOM | 2674 | CA | MET | A | 662 | 18.856 | 22.498 | 59.427 | 1.00 | 20.59 | C |
| ATOM | 2675 | C | MET | A | 662 | 20.224 | 22.242 | 58.839 | 1.00 | 20.71 | C |
| ATOM | 2676 | O | MET | A | 662 | 20.404 | 21.306 | 58.065 | 1.00 | 21.03 | O |
| ATOM | 2677 | CB | MET | A | 662 | 18.521 | 21.371 | 60.405 | 1.00 | 20.43 | C |
| ATOM | 2678 | CG | MET | A | 662 | 17.132 | 21.454 | 61.032 | 1.00 | 20.74 | C |
| ATOM | 2679 | SD | MET | A | 662 | 16.700 | 19.925 | 61.885 | 1.00 | 21.48 | S |
| ATOM | 2680 | CE | MET | A | 662 | 16.728 | 18.758 | 60.523 | 1.00 | 19.88 | C |
| ATOM | 2681 | N | ARG | A | 663 | 21.186 | 23.080 | 59.215 | 1.00 | 20.95 | N |
| ATOM | 2682 | CA | ARG | A | 663 | 22.593 | 22.836 | 58.918 | 1.00 | 21.28 | C |
| ATOM | 2683 | C | ARG | A | 663 | 23.053 | 21.526 | 59.572 | 1.00 | 21.63 | C |
| ATOM | 2684 | O | ARG | A | 663 | 22.817 | 21.290 | 60.760 | 1.00 | 21.66 | O |
| ATOM | 2685 | CB | ARG | A | 663 | 23.441 | 24.018 | 59.404 | 1.00 | 21.23 | C |
| ATOM | 2686 | CG | ARG | A | 663 | 24.950 | 23.768 | 59.456 | 1.00 | 21.19 | C |
| ATOM | 2687 | CD | ARG | A | 663 | 25.677 | 24.975 | 60.017 | 1.00 | 21.11 | C |
| ATOM | 2688 | NE | ARG | A | 663 | 27.062 | 24.684 | 60.368 | 1.00 | 21.85 | N |
| ATOM | 2689 | CZ | ARG | A | 663 | 27.962 | 25.608 | 60.697 | 1.00 | 21.81 | C |
| ATOM | 2690 | NH1 | ARG | A | 663 | 27.631 | 26.891 | 60.713 | 1.00 | 21.56 | N |
| ATOM | 2691 | NH2 | ARG | A | 663 | 29.200 | 25.253 | 61.001 | 1.00 | 21.18 | N |
| ATOM | 2692 | N | GLY | A | 664 | 23.708 | 20.680 | 58.785 | 1.00 | 22.13 | N |
| ATOM | 2693 | CA | GLY | A | 664 | 24.185 | 19.385 | 59.262 | 1.00 | 22.61 | C |
| ATOM | 2694 | C | GLY | A | 664 | 25.332 | 18.848 | 58.439 | 1.00 | 22.96 | C |
| ATOM | 2695 | O | GLY | A | 664 | 25.952 | 19.585 | 57.674 | 1.00 | 22.99 | O |
| ATOM | 2696 | N | LEU | A | 665 | 25.595 | 17.553 | 58.580 | 1.00 | 23.62 | N |
| ATOM | 2697 | CA | LEU | A | 665 | 26.810 | 16.942 | 58.037 | 1.00 | 24.16 | C |
| ATOM | 2698 | C | LEU | A | 665 | 26.528 | 15.739 | 57.146 | 1.00 | 24.59 | C |
| ATOM | 2699 | O | LEU | A | 665 | 27.218 | 14.717 | 57.211 | 1.00 | 24.72 | O |
| ATOM | 2700 | CB | LEU | A | 665 | 27.762 | 16.568 | 59.178 | 1.00 | 24.04 | C |
| ATOM | 2701 | CG | LEU | A | 665 | 28.356 | 17.787 | 59.885 | 1.00 | 24.10 | C |
| ATOM | 2702 | CD1 | LEU | A | 665 | 28.753 | 17.469 | 61.318 | 1.00 | 24.23 | C |
| ATOM | 2703 | CD2 | LEU | A | 665 | 29.531 | 18.344 | 59.089 | 1.00 | 24.60 | C |
| ATOM | 2704 | N | THR | A | 666 | 25.515 | 15.884 | 56.302 | 1.00 | 25.02 | N |
| ATOM | 2705 | CA | THR | A | 666 | 25.132 | 14.850 | 55.353 | 1.00 | 25.40 | C |
| ATOM | 2706 | C | THR | A | 666 | 25.509 | 15.262 | 53.925 | 1.00 | 25.57 | C |
| ATOM | 2707 | O | THR | A | 666 | 25.310 | 14.496 | 52.981 | 1.00 | 25.91 | O |
| ATOM | 2708 | CB | THR | A | 666 | 23.612 | 14.594 | 55.411 | 1.00 | 25.43 | C |
| ATOM | 2709 | OG1 | THR | A | 666 | 22.916 | 15.792 | 55.049 | 1.00 | 26.40 | O |
| ATOM | 2710 | CG2 | THR | A | 666 | 23.176 | 14.178 | 56.809 | 1.00 | 25.17 | C |
| ATOM | 2711 | N | GLY | A | 667 | 26.047 | 16.473 | 53.775 | 1.00 | 25.67 | N |
| ATOM | 2712 | CA | GLY | A | 667 | 26.295 | 17.076 | 52.463 | 1.00 | 25.62 | C |
| ATOM | 2713 | C | GLY | A | 667 | 25.010 | 17.471 | 51.760 | 1.00 | 25.70 | C |
| ATOM | 2714 | O | GLY | A | 667 | 25.036 | 17.989 | 50.650 | 1.00 | 26.03 | O |
| ATOM | 2715 | N | ASP | A | 668 | 23.891 | 17.237 | 52.439 | 1.00 | 25.81 | N |
| ATOM | 2716 | CA | ASP | A | 668 | 22.544 | 17.320 | 51.881 | 1.00 | 25.57 | C |
| ATOM | 2717 | C | ASP | A | 668 | 21.704 | 18.321 | 52.642 | 1.00 | 24.85 | C |
| ATOM | 2718 | O | ASP | A | 668 | 21.990 | 18.623 | 53.792 | 1.00 | 24.51 | O |
| ATOM | 2719 | CB | ASP | A | 668 | 21.852 | 15.962 | 52.039 | 1.00 | 25.81 | C |
| ATOM | 2720 | CG | ASP | A | 668 | 22.096 | 15.049 | 50.879 | 1.00 | 27.43 | C |
| ATOM | 2721 | OD1 | ASP | A | 668 | 21.783 | 15.440 | 49.743 | 1.00 | 29.81 | O |
| ATOM | 2722 | OD2 | ASP | A | 668 | 22.590 | 13.928 | 51.091 | 1.00 | 30.16 | O |
| ATOM | 2723 | N | LEU | A | 669 | 20.634 | 18.793 | 52.010 | 1.00 | 24.22 | N |
| ATOM | 2724 | CA | LEU | A | 669 | 19.634 | 19.575 | 52.715 | 1.00 | 23.55 | C |
| ATOM | 2725 | C | LEU | A | 669 | 18.783 | 18.694 | 53.629 | 1.00 | 23.26 | C |
| ATOM | 2726 | O | LEU | A | 669 | 18.132 | 17.757 | 53.174 | 1.00 | 23.44 | O |
| ATOM | 2727 | CB | LEU | A | 669 | 18.745 | 20.346 | 51.740 | 1.00 | 23.26 | C |
| ATOM | 2728 | CG | LEU | A | 669 | 17.776 | 21.317 | 52.417 | 1.00 | 22.42 | C |
| ATOM | 2729 | CD1 | LEU | A | 669 | 18.528 | 22.433 | 53.122 | 1.00 | 20.50 | C |
| ATOM | 2730 | CD2 | LEU | A | 669 | 16.798 | 21.876 | 51.408 | 1.00 | 21.11 | C |
| ATOM | 2731 | N | GLN | A | 670 | 18.794 | 19.017 | 54.917 | 1.00 | 22.85 | N |
| ATOM | 2732 | CA | GLN | A | 670 | 18.035 | 18.278 | 55.918 | 1.00 | 22.37 | C |
| ATOM | 2733 | C | GLN | A | 670 | 16.863 | 19.117 | 56.402 | 1.00 | 22.15 | C |
| ATOM | 2734 | O | GLN | A | 670 | 17.030 | 20.274 | 56.800 | 1.00 | 21.78 | O |
| ATOM | 2735 | CB | GLN | A | 670 | 18.927 | 17.914 | 57.101 | 1.00 | 22.48 | C |
| ATOM | 2736 | CG | GLN | A | 670 | 20.092 | 16.992 | 56.762 | 1.00 | 22.25 | C |
| ATOM | 2737 | CD | GLN | A | 670 | 20.767 | 16.456 | 58.009 | 1.00 | 22.43 | C |
| ATOM | 2738 | OE1 | GLN | A | 670 | 21.860 | 16.900 | 58.377 | 1.00 | 22.73 | O |
| ATOM | 2739 | NE2 | GLN | A | 670 | 20.111 | 15.513 | 58.680 | 1.00 | 20.28 | N |
| ATOM | 2740 | N | VAL | A | 671 | 15.675 | 18.522 | 56.370 | 1.00 | 22.00 | N |
| ATOM | 2741 | CA | VAL | A | 671 | 14.447 | 19.244 | 56.682 | 1.00 | 21.78 | C |
| ATOM | 2742 | C | VAL | A | 671 | 13.576 | 18.462 | 57.669 | 1.00 | 21.87 | C |
| ATOM | 2743 | O | VAL | A | 671 | 13.274 | 17.282 | 57.448 | 1.00 | 21.96 | O |
| ATOM | 2744 | CB | VAL | A | 671 | 13.634 | 19.549 | 55.400 | 1.00 | 21.71 | C |
| ATOM | 2745 | CG1 | VAL | A | 671 | 12.514 | 20.536 | 55.704 | 1.00 | 21.98 | C |
| ATOM | 2746 | CG2 | VAL | A | 671 | 14.541 | 20.091 | 54.292 | 1.00 | 21.18 | C |
| ATOM | 2747 | N | ALA | A | 672 | 13.177 | 19.131 | 58.752 | 1.00 | 21.64 | N |
| ATOM | 2748 | CA | ALA | A | 672 | 12.262 | 18.556 | 59.737 | 1.00 | 21.69 | C |
| ATOM | 2749 | C | ALA | A | 672 | 10.855 | 19.139 | 59.582 | 1.00 | 21.84 | C |
| ATOM | 2750 | O | ALA | A | 672 | 10.696 | 20.280 | 59.137 | 1.00 | 21.55 | O |
| ATOM | 2751 | CB | ALA | A | 672 | 12.783 | 18.798 | 61.148 | 1.00 | 21.50 | C |
| ATOM | 2752 | N | THR | A | 673 | 9.845 | 18.359 | 59.968 | 1.00 | 21.95 | N |
| ATOM | 2753 | CA | THR | A | 673 | 8.449 | 18.778 | 59.873 | 1.00 | 22.19 | C |
| ATOM | 2754 | C | THR | A | 673 | 7.707 | 18.753 | 61.220 | 1.00 | 22.46 | C |
| ATOM | 2755 | O | THR | A | 673 | 7.771 | 17.760 | 61.958 | 1.00 | 22.31 | O |
| ATOM | 2756 | CB | THR | A | 673 | 7.690 | 17.912 | 58.840 | 1.00 | 22.29 | C |
| ATOM | 2757 | OG1 | THR | A | 673 | 8.326 | 18.033 | 57.558 | 1.00 | 21.98 | O |
| ATOM | 2758 | CG2 | THR | A | 673 | 6.229 | 18.353 | 58.722 | 1.00 | 22.10 | C |
| ATOM | 2759 | N | SER | A | 674 | 7.001 | 19.851 | 61.516 | 1.00 | 22.87 | N |
| ATOM | 2760 | CA | SER | A | 674 | 6.187 | 19.994 | 62.731 | 1.00 | 23.25 | C |
| ATOM | 2761 | C | SER | A | 674 | 4.712 | 20.205 | 62.396 | 1.00 | 23.80 | C |
| ATOM | 2762 | O | SER | A | 674 | 4.378 | 20.992 | 61.508 | 1.00 | 23.85 | O |
| ATOM | 2763 | CB | SER | A | 674 | 6.677 | 21.178 | 63.572 | 1.00 | 23.29 | C |
| ATOM | 2764 | OG | SER | A | 674 | 5.805 | 21.444 | 64.666 | 1.00 | 23.16 | O |
| ATOM | 2765 | N | LYS | A | 675 | 3.839 | 19.526 | 63.139 | 1.00 | 24.35 | N |
| ATOM | 2766 | CA | LYS | A | 675 | 2.396 | 19.597 | 62.919 | 1.00 | 24.95 | C |
| ATOM | 2767 | C | LYS | A | 675 | 1.693 | 20.470 | 63.954 | 1.00 | 25.01 | C |
| ATOM | 2768 | O | LYS | A | 675 | 0.595 | 20.975 | 63.703 | 1.00 | 25.02 | O |
| ATOM | 2769 | CB | LYS | A | 675 | 1.781 | 18.195 | 62.944 | 1.00 | 25.23 | C |
| ATOM | 2770 | CG | LYS | A | 675 | 2.258 | 17.254 | 61.847 | 1.00 | 26.53 | C |
| ATOM | 2771 | CD | LYS | A | 675 | 1.666 | 17.619 | 60.502 | 1.00 | 28.36 | C |
| ATOM | 2772 | CE | LYS | A | 675 | 1.979 | 16.559 | 59.458 | 1.00 | 29.29 | C |
| ATOM | 2773 | NZ | LYS | A | 675 | 1.822 | 17.147 | 58.099 | 1.00 | 30.40 | N |
| ATOM | 2774 | N | ASP | A | 676 | 2.324 | 20.628 | 65.117 | 1.00 | 24.96 | N |
| ATOM | 2775 | CA | ASP | A | 676 | 1.768 | 21.413 | 66.217 | 1.00 | 24.75 | C |
| ATOM | 2776 | C | ASP | A | 676 | 2.431 | 22.785 | 66.372 | 1.00 | 24.88 | C |
| ATOM | 2777 | O | ASP | A | 676 | 2.435 | 23.370 | 67.462 | 1.00 | 25.11 | O |
| ATOM | 2778 | CB | ASP | A | 676 | 1.827 | 20.623 | 67.532 | 1.00 | 24.72 | C |
| ATOM | 2779 | CG | ASP | A | 676 | 3.233 | 20.108 | 67.866 | 1.00 | 25.45 | C |
| ATOM | 2780 | OD1 | ASP | A | 676 | 4.217 | 20.456 | 67.162 | 1.00 | 26.18 | O |
| ATOM | 2781 | OD2 | ASP | A | 676 | 3.344 | 19.341 | 68.849 | 1.00 | 24.89 | O |
| ATOM | 2782 | N | GLY | A | 677 | 2.996 | 23.291 | 65.281 | 1.00 | 24.91 | N |
| ATOM | 2783 | CA | GLY | A | 677 | 3.503 | 24.658 | 65.239 | 1.00 | 25.05 | C |
| ATOM | 2784 | C | GLY | A | 677 | 4.827 | 24.888 | 65.936 | 1.00 | 25.13 | C |
| ATOM | 2785 | O | GLY | A | 677 | 5.004 | 25.891 | 66.625 | 1.00 | 25.22 | O |
| ATOM | 2786 | N | GLY | A | 678 | 5.757 | 23.957 | 65.750 | 1.00 | 25.28 | N |
| ATOM | 2787 | CA | GLY | A | 678 | 7.123 | 24.134 | 66.217 | 1.00 | 25.26 | C |
| ATOM | 2788 | C | GLY | A | 678 | 7.489 | 23.359 | 67.457 | 1.00 | 25.31 | C |
| ATOM | 2789 | O | GLY | A | 678 | 8.670 | 23.162 | 67.724 | 1.00 | 25.68 | O |
| ATOM | 2790 | N | VAL | A | 679 | 6.484 | 22.908 | 68.209 | 1.00 | 25.43 | N |
| ATOM | 2791 | CA | VAL | A | 679 | 6.710 | 22.215 | 69.484 | 1.00 | 25.33 | C |
| ATOM | 2792 | C | VAL | A | 679 | 7.248 | 20.786 | 69.309 | 1.00 | 25.61 | C |
| ATOM | 2793 | O | VAL | A | 679 | 8.230 | 20.412 | 69.951 | 1.00 | 25.80 | O |
| ATOM | 2794 | CB | VAL | A | 679 | 5.442 | 22.221 | 70.389 | 1.00 | 25.26 | C |
| ATOM | 2795 | CG1 | VAL | A | 679 | 5.766 | 21.705 | 71.790 | 1.00 | 24.24 | C |
| ATOM | 2796 | CG2 | VAL | A | 679 | 4.848 | 23.621 | 70.475 | 1.00 | 25.13 | C |
| ATOM | 2797 | N | THR | A | 680 | 6.611 | 20.004 | 68.443 | 1.00 | 25.77 | N |
| ATOM | 2798 | CA | THR | A | 680 | 6.972 | 18.599 | 68.219 | 1.00 | 26.08 | C |
| ATOM | 2799 | C | THR | A | 680 | 7.413 | 18.408 | 66.772 | 1.00 | 25.99 | C |
| ATOM | 2800 | O | THR | A | 680 | 7.019 | 19.171 | 65.894 | 1.00 | 25.78 | O |
| ATOM | 2801 | CB | THR | A | 680 | 5.780 | 17.645 | 68.592 | 1.00 | 26.26 | C |
| ATOM | 2802 | OG1 | THR | A | 680 | 5.676 | 17.548 | 70.019 | 1.00 | 26.83 | O |
| ATOM | 2803 | CG2 | THR | A | 680 | 5.949 | 16.233 | 68.033 | 1.00 | 27.03 | C |
| ATOM | 2804 | N | TRP | A | 681 | 8.241 | 17.393 | 66.533 | 1.00 | 26.13 | N |
| ATOM | 2805 | CA | TRP | A | 681 | 8.744 | 17.101 | 65.195 | 1.00 | 26.30 | C |
| ATOM | 2806 | C | TRP | A | 681 | 8.414 | 15.675 | 64.784 | 1.00 | 27.08 | C |
| ATOM | 2807 | O | TRP | A | 681 | 8.415 | 14.766 | 65.619 | 1.00 | 27.10 | O |
| ATOM | 2808 | CB | TRP | A | 681 | 10.250 | 17.350 | 65.128 | 1.00 | 25.70 | C |
| ATOM | 2809 | CG | TRP | A | 681 | 10.596 | 18.753 | 65.500 | 1.00 | 25.23 | C |
| ATOM | 2810 | CD1 | TRP | A | 681 | 11.032 | 19.198 | 66.713 | 1.00 | 23.95 | C |
| ATOM | 2811 | CD2 | TRP | A | 681 | 10.492 | 19.908 | 64.659 | 1.00 | 24.66 | C |
| ATOM | 2812 | NE1 | TRP | A | 681 | 11.227 | 20.556 | 66.674 | 1.00 | 24.01 | N |
| ATOM | 2813 | CE2 | TRP | A | 681 | 10.900 | 21.018 | 65.427 | 1.00 | 24.30 | C |
| ATOM | 2814 | CE3 | TRP | A | 681 | 10.103 | 20.110 | 63.325 | 1.00 | 23.66 | C |
| ATOM | 2815 | CZ2 | TRP | A | 681 | 10.927 | 22.318 | 64.909 | 1.00 | 25.23 | C |
| ATOM | 2816 | CZ3 | TRP | A | 681 | 10.129 | 21.396 | 62.812 | 1.00 | 24.13 | C |
| ATOM | 2817 | CH2 | TRP | A | 681 | 10.538 | 22.486 | 63.603 | 1.00 | 24.80 | C |
| ATOM | 2818 | N | GLU | A | 682 | 8.123 | 15.489 | 63.498 | 1.00 | 27.96 | N |
| ATOM | 2819 | CA | GLU | A | 682 | 7.829 | 14.162 | 62.950 | 1.00 | 28.91 | C |
| ATOM | 2820 | C | GLU | A | 682 | 9.044 | 13.225 | 62.981 | 1.00 | 29.28 | C |
| ATOM | 2821 | O | GLU | A | 682 | 10.184 | 13.675 | 63.076 | 1.00 | 29.32 | O |
| ATOM | 2822 | CB | GLU | A | 682 | 7.266 | 14.278 | 61.530 | 1.00 | 28.96 | C |
| ATOM | 2823 | CG | GLU | A | 682 | 5.876 | 14.912 | 61.461 | 1.00 | 30.54 | C |
| ATOM | 2824 | CD | GLU | A | 682 | 4.858 | 14.211 | 62.357 | 1.00 | 32.68 | C |
| ATOM | 2825 | OE1 | GLU | A | 682 | 4.516 | 13.037 | 62.082 | 1.00 | 33.52 | O |
| ATOM | 2826 | OE2 | GLU | A | 682 | 4.397 | 14.837 | 63.336 | 1.00 | 33.25 | O |
| ATOM | 2827 | N | LYS | A | 683 | 8.777 | 11.923 | 62.902 | 1.00 | 30.07 | N |
| ATOM | 2828 | CA | LYS | A | 683 | 9.791 | 10.872 | 63.030 | 1.00 | 30.62 | C |
| ATOM | 2829 | C | LYS | A | 683 | 11.050 | 11.128 | 62.208 | 1.00 | 30.71 | C |
| ATOM | 2830 | O | LYS | A | 683 | 12.169 | 11.057 | 62.728 | 1.00 | 30.80 | O |
| ATOM | 2831 | CB | LYS | A | 683 | 9.198 | 9.519 | 62.616 | 1.00 | 30.81 | C |
| ATOM | 2832 | CG | LYS | A | 683 | 8.600 | 8.701 | 63.753 | 1.00 | 32.31 | C |
| ATOM | 2833 | CD | LYS | A | 683 | 7.543 | 7.707 | 63.247 | 1.00 | 34.37 | C |
| ATOM | 2834 | CE | LYS | A | 683 | 8.156 | 6.543 | 62.459 | 1.00 | 35.83 | C |
| ATOM | 2835 | NZ | LYS | A | 683 | 7.128 | 5.539 | 62.017 | 1.00 | 35.88 | N |
| ATOM | 2836 | N | ASP | A | 684 | 10.849 | 11.430 | 60.928 | 1.00 | 30.56 | N |
| ATOM | 2837 | CA | ASP | A | 684 | 11.922 | 11.384 | 59.945 | 1.00 | 30.58 | C |
| ATOM | 2838 | C | ASP | A | 684 | 12.362 | 12.743 | 59.410 | 1.00 | 30.11 | C |
| ATOM | 2839 | O | ASP | A | 684 | 11.550 | 13.647 | 59.201 | 1.00 | 29.98 | O |
| ATOM | 2840 | CB | ASP | A | 684 | 11.521 | 10.467 | 58.786 | 1.00 | 30.74 | C |
| ATOM | 2841 | CG | ASP | A | 684 | 11.360 | 9.018 | 59.221 | 1.00 | 31.96 | C |
| ATOM | 2842 | OD1 | ASP | A | 684 | 12.247 | 8.504 | 59.947 | 1.00 | 33.01 | O |
| ATOM | 2843 | OD2 | ASP | A | 684 | 10.346 | 8.394 | 58.835 | 1.00 | 31.88 | O |
| ATOM | 2844 | N | ILE | A | 685 | 13.667 | 12.862 | 59.201 | 1.00 | 29.62 | N |
| ATOM | 2845 | CA | ILE | A | 685 | 14.262 | 14.033 | 58.587 | 1.00 | 29.16 | C |
| ATOM | 2846 | C | ILE | A | 685 | 14.356 | 13.753 | 57.094 | 1.00 | 28.90 | C |
| ATOM | 2847 | O | ILE | A | 685 | 15.032 | 12.807 | 56.669 | 1.00 | 28.84 | O |
| ATOM | 2848 | CB | ILE | A | 685 | 15.660 | 14.342 | 59.186 | 1.00 | 29.07 | C |
| ATOM | 2849 | CG1 | ILE | A | 685 | 15.540 | 14.663 | 60.679 | 1.00 | 29.10 | C |
| ATOM | 2850 | CG2 | ILE | A | 685 | 16.318 | 15.509 | 58.460 | 1.00 | 29.66 | C |
| ATOM | 2851 | CD1 | ILE | A | 685 | 16.869 | 14.938 | 61.369 | 1.00 | 29.03 | C |
| ATOM | 2852 | N | LYS | A | 686 | 13.647 | 14.558 | 56.306 | 1.00 | 28.48 | N |
| ATOM | 2853 | CA | LYS | A | 686 | 13.695 | 14.462 | 54.848 | 1.00 | 28.13 | C |
| ATOM | 2854 | C | LYS | A | 686 | 15.033 | 15.007 | 54.340 | 1.00 | 27.72 | C |
| ATOM | 2855 | O | LYS | A | 686 | 15.547 | 15.994 | 54.862 | 1.00 | 27.96 | O |
| ATOM | 2856 | CB | LYS | A | 686 | 12.506 | 15.214 | 54.235 | 1.00 | 28.14 | C |
| ATOM | 2857 | CG | LYS | A | 686 | 12.456 | 15.253 | 52.710 | 1.00 | 28.62 | C |
| ATOM | 2858 | CD | LYS | A | 686 | 11.921 | 13.973 | 52.087 | 1.00 | 28.85 | C |
| ATOM | 2859 | CE | LYS | A | 686 | 11.976 | 14.058 | 50.564 | 1.00 | 29.14 | C |
| ATOM | 2860 | NZ | LYS | A | 686 | 11.580 | 12.784 | 49.911 | 1.00 | 28.93 | N |
| ATOM | 2861 | N | ARG | A | 687 | 15.610 | 14.342 | 53.347 | 1.00 | 27.22 | N |
| ATOM | 2862 | CA | ARG | A | 687 | 16.865 | 14.790 | 52.751 | 1.00 | 26.84 | C |
| ATOM | 2863 | C | ARG | A | 687 | 16.721 | 15.030 | 51.255 | 1.00 | 27.11 | C |
| ATOM | 2864 | O | ARG | A | 687 | 16.130 | 14.217 | 50.540 | 1.00 | 27.10 | O |
| ATOM | 2865 | CB | ARG | A | 687 | 17.994 | 13.799 | 53.025 | 1.00 | 26.44 | C |
| ATOM | 2866 | CG | ARG | A | 687 | 18.744 | 14.067 | 54.315 | 1.00 | 25.69 | C |
| ATOM | 2867 | CD | ARG | A | 687 | 19.743 | 12.971 | 54.621 | 1.00 | 23.73 | C |
| ATOM | 2868 | NE | ARG | A | 687 | 20.773 | 12.885 | 53.588 | 1.00 | 23.79 | N |
| ATOM | 2869 | CZ | ARG | A | 687 | 21.680 | 11.916 | 53.499 | 1.00 | 23.09 | C |
| ATOM | 2870 | NH1 | ARG | A | 687 | 21.707 | 10.925 | 54.381 | 1.00 | 23.76 | N |
| ATOM | 2871 | NH2 | ARG | A | 687 | 22.563 | 11.935 | 52.520 | 1.00 | 22.74 | N |
| ATOM | 2872 | N | TYR | A | 688 | 17.263 | 16.156 | 50.796 | 1.00 | 27.26 | N |
| ATOM | 2873 | CA | TYR | A | 688 | 17.207 | 16.537 | 49.390 | 1.00 | 27.52 | C |
| ATOM | 2874 | C | TYR | A | 688 | 18.610 | 16.628 | 48.783 | 1.00 | 27.88 | C |
| ATOM | 2875 | O | TYR | A | 688 | 19.317 | 17.619 | 48.992 | 1.00 | 27.88 | O |
| ATOM | 2876 | CB | TYR | A | 688 | 16.470 | 17.872 | 49.213 | 1.00 | 27.16 | C |
| ATOM | 2877 | CG | TYR | A | 688 | 15.005 | 17.840 | 49.597 | 1.00 | 26.62 | C |
| ATOM | 2878 | CD1 | TYR | A | 688 | 14.038 | 17.389 | 48.698 | 1.00 | 25.45 | C |
| ATOM | 2879 | CD2 | TYR | A | 688 | 14.586 | 18.281 | 50.855 | 1.00 | 25.54 | C |
| ATOM | 2880 | CE1 | TYR | A | 688 | 12.684 | 17.366 | 49.050 | 1.00 | 25.89 | C |
| ATOM | 2881 | CE2 | TYR | A | 688 | 13.246 | 18.264 | 51.216 | 1.00 | 25.60 | C |
| ATOM | 2882 | CZ | TYR | A | 688 | 12.296 | 17.805 | 50.310 | 1.00 | 26.63 | C |
| ATOM | 2883 | OH | TYR | A | 688 | 10.963 | 17.782 | 50.668 | 1.00 | 26.80 | O |
| ATOM | 2884 | N | PRO | A | 689 | 19.019 | 15.589 | 48.029 | 1.00 | 28.11 | N |
| ATOM | 2885 | CA | PRO | A | 689 | 20.294 | 15.618 | 47.304 | 1.00 | 28.10 | C |
| ATOM | 2886 | C | PRO | A | 689 | 20.379 | 16.735 | 46.247 | 1.00 | 28.25 | C |
| ATOM | 2887 | O | PRO | A | 689 | 21.476 | 17.050 | 45.770 | 1.00 | 28.17 | O |
| ATOM | 2888 | CB | PRO | A | 689 | 20.368 | 14.229 | 46.654 | 1.00 | 28.12 | C |
| ATOM | 2889 | CG | PRO | A | 689 | 18.952 | 13.726 | 46.624 | 1.00 | 28.17 | C |
| ATOM | 2890 | CD | PRO | A | 689 | 18.297 | 14.314 | 47.839 | 1.00 | 28.24 | C |
| ATOM | 2891 | N | GLN | A | 690 | 19.238 | 17.331 | 45.900 | 1.00 | 28.21 | N |
| ATOM | 2892 | CA | GLN | A | 690 | 19.198 | 18.448 | 44.948 | 1.00 | 28.24 | C |
| ATOM | 2893 | C | GLN | A | 690 | 19.812 | 19.731 | 45.525 | 1.00 | 28.12 | C |
| ATOM | 2894 | O | GLN | A | 690 | 20.129 | 20.661 | 44.779 | 1.00 | 28.20 | O |
| ATOM | 2895 | CB | GLN | A | 690 | 17.761 | 18.739 | 44.481 | 1.00 | 28.38 | C |
| ATOM | 2896 | CG | GLN | A | 690 | 17.037 | 17.593 | 43.773 | 1.00 | 29.01 | C |
| ATOM | 2897 | CD | GLN | A | 690 | 16.384 | 16.611 | 44.744 | 1.00 | 30.57 | C |
| ATOM | 2898 | OE1 | GLN | A | 690 | 16.304 | 16.861 | 45.952 | 1.00 | 30.90 | O |
| ATOM | 2899 | NE2 | GLN | A | 690 | 15.930 | 15.478 | 44.217 | 1.00 | 31.01 | N |
| ATOM | 2900 | N | VAL | A | 691 | 19.962 | 19.791 | 46.847 | 1.00 | 27.77 | N |
| ATOM | 2901 | CA | VAL | A | 691 | 20.527 | 20.974 | 47.490 | 1.00 | 27.53 | C |
| ATOM | 2902 | C | VAL | A | 691 | 21.675 | 20.607 | 48.424 | 1.00 | 27.48 | C |
| ATOM | 2903 | O | VAL | A | 691 | 21.488 | 19.904 | 49.420 | 1.00 | 27.36 | O |
| ATOM | 2904 | CB | VAL | A | 691 | 19.468 | 21.800 | 48.265 | 1.00 | 27.50 | C |
| ATOM | 2905 | CG1 | VAL | A | 691 | 20.013 | 23.179 | 48.580 | 1.00 | 27.73 | C |
| ATOM | 2906 | CG2 | VAL | A | 691 | 18.180 | 21.929 | 47.471 | 1.00 | 27.31 | C |
| ATOM | 2907 | N | LYS | A | 692 | 22.860 | 21.107 | 48.091 | 1.00 | 27.46 | N |
| ATOM | 2908 | CA | LYS | A | 692 | 24.068 | 20.805 | 48.838 | 1.00 | 27.30 | C |
| ATOM | 2909 | C | LYS | A | 692 | 24.110 | 21.610 | 50.117 | 1.00 | 27.02 | C |
| ATOM | 2910 | O | LYS | A | 692 | 23.570 | 22.720 | 50.177 | 1.00 | 27.33 | O |
| ATOM | 2911 | CB | LYS | A | 692 | 25.318 | 21.110 | 47.999 | 1.00 | 27.52 | C |
| ATOM | 2912 | CG | LYS | A | 692 | 25.509 | 20.228 | 46.773 | 1.00 | 27.99 | C |
| ATOM | 2913 | CD | LYS | A | 692 | 25.791 | 18.782 | 47.170 | 1.00 | 29.81 | C |
| ATOM | 2914 | CE | LYS | A | 692 | 26.051 | 17.912 | 45.950 | 1.00 | 29.90 | C |
| ATOM | 2915 | NZ | LYS | A | 692 | 26.333 | 16.525 | 46.372 | 1.00 | 30.59 | N |
| ATOM | 2916 | N | ASP | A | 693 | 24.756 | 21.038 | 51.131 | 1.00 | 26.46 | N |
| ATOM | 2917 | CA | ASP | A | 693 | 25.038 | 21.724 | 52.389 | 1.00 | 26.08 | C |
| ATOM | 2918 | C | ASP | A | 693 | 26.472 | 21.404 | 52.801 | 1.00 | 25.73 | C |
| ATOM | 2919 | O | ASP | A | 693 | 26.805 | 20.260 | 53.105 | 1.00 | 25.62 | O |
| ATOM | 2920 | CB | ASP | A | 693 | 24.022 | 21.308 | 53.464 | 1.00 | 26.08 | C |
| ATOM | 2921 | CG | ASP | A | 693 | 24.465 | 21.651 | 54.882 | 1.00 | 26.58 | C |
| ATOM | 2922 | OD1 | ASP | A | 693 | 25.171 | 22.661 | 55.093 | 1.00 | 27.15 | O |
| ATOM | 2923 | OD2 | ASP | A | 693 | 24.084 | 20.899 | 55.801 | 1.00 | 27.65 | O |
| ATOM | 2924 | N | VAL | A | 694 | 27.322 | 22.424 | 52.791 | 1.00 | 25.49 | N |
| ATOM | 2925 | CA | VAL | A | 694 | 28.745 | 22.248 | 53.107 | 1.00 | 25.17 | C |
| ATOM | 2926 | C | VAL | A | 694 | 29.037 | 22.589 | 54.576 | 1.00 | 25.16 | C |
| ATOM | 2927 | O | VAL | A | 694 | 30.163 | 22.963 | 54.932 | 1.00 | 25.29 | O |
| ATOM | 2928 | CB | VAL | A | 694 | 29.651 | 23.065 | 52.144 | 1.00 | 25.19 | C |
| ATOM | 2929 | CG1 | VAL | A | 694 | 29.335 | 22.720 | 50.682 | 1.00 | 24.30 | C |
| ATOM | 2930 | CG2 | VAL | A | 694 | 29.502 | 24.566 | 52.388 | 1.00 | 24.85 | C |
| ATOM | 2931 | N | TYR | A | 695 | 28.004 | 22.448 | 55.415 | 1.00 | 24.76 | N |
| ATOM | 2932 | CA | TYR | A | 695 | 28.067 | 22.709 | 56.861 | 1.00 | 24.22 | C |
| ATOM | 2933 | C | TYR | A | 695 | 28.224 | 24.200 | 57.146 | 1.00 | 23.99 | C |
| ATOM | 2934 | O | TYR | A | 695 | 29.211 | 24.662 | 57.733 | 1.00 | 23.98 | O |
| ATOM | 2935 | CB | TYR | A | 695 | 29.137 | 21.850 | 57.554 | 1.00 | 24.11 | C |
| ATOM | 2936 | CG | TYR | A | 695 | 29.067 | 21.849 | 59.064 | 1.00 | 24.22 | C |
| ATOM | 2937 | CD1 | TYR | A | 695 | 27.879 | 21.530 | 59.733 | 1.00 | 24.31 | C |
| ATOM | 2938 | CD2 | TYR | A | 695 | 30.193 | 22.156 | 59.831 | 1.00 | 23.99 | C |
| ATOM | 2939 | CE1 | TYR | A | 695 | 27.812 | 21.525 | 61.128 | 1.00 | 23.96 | C |
| ATOM | 2940 | CE2 | TYR | A | 695 | 30.139 | 22.153 | 61.226 | 1.00 | 23.84 | C |
| ATOM | 2941 | CZ | TYR | A | 695 | 28.949 | 21.834 | 61.865 | 1.00 | 24.02 | C |
| ATOM | 2942 | OH | TYR | A | 695 | 28.898 | 21.828 | 63.239 | 1.00 | 23.92 | O |
| ATOM | 2943 | N | VAL | A | 696 | 27.217 | 24.947 | 56.711 | 1.00 | 23.62 | N |
| ATOM | 2944 | CA | VAL | A | 696 | 27.194 | 26.394 | 56.834 | 1.00 | 22.96 | C |
| ATOM | 2945 | C | VAL | A | 696 | 25.730 | 26.837 | 56.967 | 1.00 | 22.54 | C |
| ATOM | 2946 | O | VAL | A | 696 | 24.817 | 26.106 | 56.578 | 1.00 | 22.03 | O |
| ATOM | 2947 | CB | VAL | A | 696 | 27.936 | 27.068 | 55.631 | 1.00 | 22.87 | C |
| ATOM | 2948 | CG1 | VAL | A | 696 | 27.168 | 26.878 | 54.309 | 1.00 | 23.20 | C |
| ATOM | 2949 | CG2 | VAL | A | 696 | 28.220 | 28.540 | 55.903 | 1.00 | 22.58 | C |
| ATOM | 2950 | N | GLN | A | 697 | 25.522 | 28.018 | 57.543 | 1.00 | 22.28 | N |
| ATOM | 2951 | CA | GLN | A | 697 | 24.184 | 28.575 | 57.751 | 1.00 | 22.24 | C |
| ATOM | 2952 | C | GLN | A | 697 | 23.454 | 28.865 | 56.434 | 1.00 | 22.48 | C |
| ATOM | 2953 | O | GLN | A | 697 | 24.079 | 29.148 | 55.403 | 1.00 | 22.65 | O |
| ATOM | 2954 | CB | GLN | A | 697 | 24.281 | 29.839 | 58.615 | 1.00 | 22.00 | C |
| ATOM | 2955 | CG | GLN | A | 697 | 22.972 | 30.572 | 58.912 | 1.00 | 21.27 | C |
| ATOM | 2956 | CD | GLN | A | 697 | 22.686 | 31.719 | 57.949 | 1.00 | 20.33 | C |
| ATOM | 2957 | OE1 | GLN | A | 697 | 23.283 | 31.822 | 56.873 | 1.00 | 20.66 | O |
| ATOM | 2958 | NE2 | GLN | A | 697 | 21.757 | 32.579 | 58.332 | 1.00 | 19.36 | N |
| ATOM | 2959 | N | MET | A | 698 | 22.129 | 28.803 | 56.496 | 1.00 | 22.33 | N |
| ATOM | 2960 | CA | MET | A | 698 | 21.271 | 29.061 | 55.358 | 1.00 | 22.60 | C |
| ATOM | 2961 | C | MET | A | 698 | 20.074 | 29.880 | 55.801 | 1.00 | 22.71 | C |
| ATOM | 2962 | O | MET | A | 698 | 19.766 | 29.948 | 56.988 | 1.00 | 23.00 | O |
| ATOM | 2963 | CB | MET | A | 698 | 20.796 | 27.739 | 54.750 | 1.00 | 22.54 | C |
| ATOM | 2964 | CG | MET | A | 698 | 19.911 | 26.898 | 55.667 | 1.00 | 22.25 | C |
| ATOM | 2965 | SD | MET | A | 698 | 19.650 | 25.232 | 55.036 | 1.00 | 23.52 | S |
| ATOM | 2966 | CE | MET | A | 698 | 21.133 | 24.397 | 55.613 | 1.00 | 20.73 | C |
| ATOM | 2967 | N | SER | A | 699 | 19.406 | 30.514 | 54.847 | 1.00 | 23.03 | N |
| ATOM | 2968 | CA | SER | A | 699 | 18.124 | 31.140 | 55.114 | 1.00 | 23.44 | C |
| ATOM | 2969 | C | SER | A | 699 | 17.109 | 30.716 | 54.068 | 1.00 | 23.69 | C |
| ATOM | 2970 | O | SER | A | 699 | 17.439 | 30.549 | 52.895 | 1.00 | 23.75 | O |
| ATOM | 2971 | CB | SER | A | 699 | 18.243 | 32.665 | 55.183 | 1.00 | 23.38 | C |
| ATOM | 2972 | OG | SER | A | 699 | 18.631 | 33.216 | 53.944 | 1.00 | 23.78 | O |
| ATOM | 2973 | N | ALA | A | 700 | 15.878 | 30.521 | 54.514 | 1.00 | 23.94 | N |
| ATOM | 2974 | CA | ALA | A | 700 | 14.792 | 30.143 | 53.632 | 1.00 | 24.25 | C |
| ATOM | 2975 | C | ALA | A | 700 | 13.565 | 30.960 | 53.985 | 1.00 | 24.58 | C |
| ATOM | 2976 | O | ALA | A | 700 | 13.333 | 31.269 | 55.159 | 1.00 | 24.28 | O |
| ATOM | 2977 | CB | ALA | A | 700 | 14.493 | 28.656 | 53.749 | 1.00 | 23.92 | C |
| ATOM | 2978 | N | ILE | A | 701 | 12.790 | 31.313 | 52.962 | 1.00 | 25.06 | N |
| ATOM | 2979 | CA | ILE | A | 701 | 11.541 | 32.050 | 53.151 | 1.00 | 25.62 | C |
| ATOM | 2980 | C | ILE | A | 701 | 10.404 | 31.543 | 52.267 | 1.00 | 26.12 | C |
| ATOM | 2981 | O | ILE | A | 701 | 10.615 | 31.120 | 51.127 | 1.00 | 26.25 | O |
| ATOM | 2982 | CB | ILE | A | 701 | 11.703 | 33.588 | 52.940 | 1.00 | 25.70 | C |
| ATOM | 2983 | CG1 | ILE | A | 701 | 12.418 | 33.902 | 51.618 | 1.00 | 25.41 | C |
| ATOM | 2984 | CG2 | ILE | A | 701 | 12.398 | 34.237 | 54.147 | 1.00 | 25.40 | C |
| ATOM | 2985 | CD1 | ILE | A | 701 | 12.189 | 35.310 | 51.108 | 1.00 | 25.45 | C |
| ATOM | 2986 | N | HIS | A | 702 | 9.201 | 31.581 | 52.830 | 1.00 | 26.55 | N |
| ATOM | 2987 | CA | HIS | A | 702 | 7.967 | 31.384 | 52.097 | 1.00 | 27.00 | C |
| ATOM | 2988 | C | HIS | A | 702 | 7.703 | 32.597 | 51.199 | 1.00 | 27.63 | C |
| ATOM | 2989 | O | HIS | A | 702 | 7.992 | 33.738 | 51.571 | 1.00 | 27.59 | O |
| ATOM | 2990 | CB | HIS | A | 702 | 6.826 | 31.217 | 53.096 | 1.00 | 26.88 | C |
| ATOM | 2991 | CG | HIS | A | 702 | 5.516 | 30.867 | 52.471 | 1.00 | 26.99 | C |
| ATOM | 2992 | ND1 | HIS | A | 702 | 4.568 | 31.816 | 52.150 | 1.00 | 27.26 | N |
| ATOM | 2993 | CD2 | HIS | A | 702 | 4.989 | 29.672 | 52.115 | 1.00 | 27.42 | C |
| ATOM | 2994 | CE1 | HIS | A | 702 | 3.514 | 31.220 | 51.621 | 1.00 | 27.71 | C |
| ATOM | 2995 | NE2 | HIS | A | 702 | 3.745 | 29.919 | 51.588 | 1.00 | 28.48 | N |
| ATOM | 2996 | N | THR | A | 703 | 7.167 | 32.344 | 50.010 | 1.00 | 28.42 | N |
| ATOM | 2997 | CA | THR | A | 703 | 6.748 | 33.417 | 49.111 | 1.00 | 29.06 | C |
| ATOM | 2998 | C | THR | A | 703 | 5.617 | 32.953 | 48.206 | 1.00 | 29.68 | C |
| ATOM | 2999 | O | THR | A | 703 | 5.580 | 31.793 | 47.820 | 1.00 | 29.84 | O |
| ATOM | 3000 | CB | THR | A | 703 | 7.919 | 33.952 | 48.251 | 1.00 | 29.01 | C |
| ATOM | 3001 | OG1 | THR | A | 703 | 7.453 | 35.053 | 47.464 | 1.00 | 28.58 | O |
| ATOM | 3002 | CG2 | THR | A | 703 | 8.488 | 32.861 | 47.332 | 1.00 | 28.40 | C |
| ATOM | 3003 | N | MET | A | 704 | 4.708 | 33.860 | 47.864 | 1.00 | 30.37 | N |
| ATOM | 3004 | CA | MET | A | 704 | 3.579 | 33.534 | 46.992 | 1.00 | 30.95 | C |
| ATOM | 3005 | C | MET | A | 704 | 3.755 | 34.177 | 45.627 | 1.00 | 31.65 | C |
| ATOM | 3006 | O | MET | A | 704 | 3.897 | 35.400 | 45.525 | 1.00 | 32.07 | O |
| ATOM | 3007 | CB | MET | A | 704 | 2.257 | 33.994 | 47.614 | 1.00 | 30.63 | C |
| ATOM | 3008 | CG | MET | A | 704 | 1.802 | 33.165 | 48.805 | 1.00 | 30.20 | C |
| ATOM | 3009 | SD | MET | A | 704 | 1.565 | 31.422 | 48.419 | 1.00 | 29.61 | S |
| ATOM | 3010 | CE | MET | A | 704 | 0.203 | 31.522 | 47.265 | 1.00 | 29.77 | C |
| ATOM | 3011 | N | HIS | A | 705 | 3.747 | 33.356 | 44.581 | 1.00 | 32.21 | N |
| ATOM | 3012 | CA | HIS | A | 705 | 3.848 | 33.863 | 43.215 | 1.00 | 32.81 | C |
| ATOM | 3013 | C | HIS | A | 705 | 2.721 | 33.373 | 42.303 | 1.00 | 33.17 | C |
| ATOM | 3014 | O | HIS | A | 705 | 2.657 | 32.196 | 41.938 | 1.00 | 33.18 | O |
| ATOM | 3015 | CB | HIS | A | 705 | 5.219 | 33.544 | 42.611 | 1.00 | 32.82 | C |
| ATOM | 3016 | CG | HIS | A | 705 | 5.522 | 34.319 | 41.368 | 1.00 | 32.82 | C |
| ATOM | 3017 | ND1 | HIS | A | 705 | 5.440 | 33.767 | 40.108 | 1.00 | 32.78 | N |
| ATOM | 3018 | CD2 | HIS | A | 705 | 5.893 | 35.610 | 41.190 | 1.00 | 32.98 | C |
| ATOM | 3019 | CE1 | HIS | A | 705 | 5.760 | 34.680 | 39.208 | 1.00 | 33.05 | C |
| ATOM | 3020 | NE2 | HIS | A | 705 | 6.035 | 35.808 | 39.838 | 1.00 | 33.05 | N |
| ATOM | 3021 | N | GLU | A | 706 | 1.827 | 34.296 | 41.964 | 1.00 | 33.91 | N |
| ATOM | 3022 | CA | GLU | A | 706 | 0.738 | 34.069 | 41.006 | 1.00 | 34.54 | C |
| ATOM | 3023 | C | GLU | A | 706 | −0.084 | 32.804 | 41.264 | 1.00 | 34.89 | C |
| ATOM | 3024 | O | GLU | A | 706 | −0.424 | 32.082 | 40.322 | 1.00 | 34.87 | O |
| ATOM | 3025 | CB | GLU | A | 706 | 1.281 | 34.041 | 39.572 | 1.00 | 34.62 | C |
| ATOM | 3026 | CG | GLU | A | 706 | 1.994 | 35.301 | 39.103 | 1.00 | 34.47 | C |
| ATOM | 3027 | CD | GLU | A | 706 | 2.406 | 35.216 | 37.637 | 1.00 | 34.59 | C |
| ATOM | 3028 | OE1 | GLU | A | 706 | 2.314 | 34.113 | 37.051 | 1.00 | 35.10 | O |
| ATOM | 3029 | OE2 | GLU | A | 706 | 2.822 | 36.247 | 37.070 | 1.00 | 33.50 | O |
| ATOM | 3030 | N | GLY | A | 707 | −0.402 | 32.526 | 42.526 | 1.00 | 35.36 | N |
| ATOM | 3031 | CA | GLY | A | 707 | −1.252 | 31.376 | 42.834 | 1.00 | 35.89 | C |
| ATOM | 3032 | C | GLY | A | 707 | −0.817 | 30.493 | 43.984 | 1.00 | 36.11 | C |
| ATOM | 3033 | O | GLY | A | 707 | −1.468 | 30.489 | 45.027 | 1.00 | 36.49 | O |
| ATOM | 3034 | N | LYS | A | 708 | 0.258 | 29.727 | 43.781 | 1.00 | 36.24 | N |
| ATOM | 3035 | CA | LYS | A | 708 | 0.838 | 28.809 | 44.796 | 1.00 | 36.25 | C |
| ATOM | 3036 | C | LYS | A | 708 | 2.226 | 28.390 | 44.300 | 1.00 | 35.76 | C |
| ATOM | 3037 | O | LYS | A | 708 | 2.420 | 28.335 | 43.081 | 1.00 | 36.02 | O |
| ATOM | 3038 | CB | LYS | A | 708 | −0.010 | 27.526 | 44.968 | 1.00 | 36.49 | C |
| ATOM | 3039 | CG | LYS | A | 708 | −1.448 | 27.671 | 45.538 | 1.00 | 37.08 | C |
| ATOM | 3040 | CD | LYS | A | 708 | −1.504 | 27.832 | 47.078 | 1.00 | 37.89 | C |
| ATOM | 3041 | CE | LYS | A | 708 | −1.406 | 26.492 | 47.814 | 1.00 | 37.82 | C |
| ATOM | 3042 | NZ | LYS | A | 708 | −1.937 | 26.587 | 49.203 | 1.00 | 37.19 | N |
| ATOM | 3043 | N | GLU | A | 709 | 3.212 | 28.118 | 45.171 | 1.00 | 35.06 | N |
| ATOM | 3044 | CA | GLU | A | 709 | 3.295 | 28.503 | 46.587 | 1.00 | 33.89 | C |
| ATOM | 3045 | C | GLU | A | 709 | 4.757 | 28.870 | 46.894 | 1.00 | 33.44 | C |
| ATOM | 3046 | O | GLU | A | 709 | 5.020 | 29.968 | 47.375 | 1.00 | 33.62 | O |
| ATOM | 3047 | CB | GLU | A | 709 | 2.810 | 27.421 | 47.559 | 1.00 | 33.76 | C |
| ATOM | 3048 | CG | GLU | A | 709 | 3.063 | 27.838 | 49.015 | 1.00 | 33.61 | C |
| ATOM | 3049 | CD | GLU | A | 709 | 2.382 | 26.986 | 50.069 | 1.00 | 33.57 | C |
| ATOM | 3050 | OE1 | GLU | A | 709 | 1.636 | 26.050 | 49.717 | 1.00 | 33.17 | O |
| ATOM | 3051 | OE2 | GLU | A | 709 | 2.597 | 27.275 | 51.271 | 1.00 | 32.91 | O |
| ATOM | 3052 | N | TYR | A | 710 | 5.690 | 27.951 | 46.629 | 1.00 | 32.24 | N |
| ATOM | 3053 | CA | TYR | A | 710 | 7.154 | 28.225 | 46.653 | 1.00 | 31.17 | C |
| ATOM | 3054 | C | TYR | A | 710 | 7.866 | 28.511 | 47.992 | 1.00 | 30.36 | C |
| ATOM | 3055 | O | TYR | A | 710 | 7.322 | 29.155 | 48.891 | 1.00 | 30.13 | O |
| ATOM | 3056 | CB | TYR | A | 710 | 7.533 | 29.332 | 45.650 | 1.00 | 31.34 | C |
| ATOM | 3057 | CG | TYR | A | 710 | 6.977 | 29.131 | 44.261 | 1.00 | 31.63 | C |
| ATOM | 3058 | CD1 | TYR | A | 710 | 7.560 | 28.218 | 43.379 | 1.00 | 31.60 | C |
| ATOM | 3059 | CD2 | TYR | A | 710 | 5.866 | 29.849 | 43.829 | 1.00 | 31.64 | C |
| ATOM | 3060 | CE1 | TYR | A | 710 | 7.044 | 28.021 | 42.106 | 1.00 | 31.66 | C |
| ATOM | 3061 | CE2 | TYR | A | 710 | 5.342 | 29.664 | 42.554 | 1.00 | 32.03 | C |
| ATOM | 3062 | CZ | TYR | A | 710 | 5.936 | 28.749 | 41.700 | 1.00 | 31.58 | C |
| ATOM | 3063 | OH | TYR | A | 710 | 5.423 | 28.565 | 40.438 | 1.00 | 31.67 | O |
| ATOM | 3064 | N | ILE | A | 711 | 9.107 | 28.029 | 48.082 | 1.00 | 29.40 | N |
| ATOM | 3065 | CA | ILE | A | 711 | 10.072 | 28.431 | 49.107 | 1.00 | 28.19 | C |
| ATOM | 3066 | C | ILE | A | 711 | 11.390 | 28.813 | 48.439 | 1.00 | 27.72 | C |
| ATOM | 3067 | O | ILE | A | 711 | 11.834 | 28.156 | 47.500 | 1.00 | 27.67 | O |
| ATOM | 3068 | CB | ILE | A | 711 | 10.350 | 27.308 | 50.137 | 1.00 | 28.32 | C |
| ATOM | 3069 | CG1 | ILE | A | 711 | 9.127 | 27.056 | 51.020 | 1.00 | 27.47 | C |
| ATOM | 3070 | CG2 | ILE | A | 711 | 11.571 | 27.654 | 51.017 | 1.00 | 27.99 | C |
| ATOM | 3071 | CD1 | ILE | A | 711 | 9.270 | 25.843 | 51.886 | 1.00 | 27.46 | C |
| ATOM | 3072 | N | ILE | A | 712 | 12.013 | 29.874 | 48.938 | 1.00 | 27.17 | N |
| ATOM | 3073 | CA | ILE | A | 712 | 13.318 | 30.310 | 48.461 | 1.00 | 26.48 | C |
| ATOM | 3074 | C | ILE | A | 712 | 14.355 | 30.076 | 49.557 | 1.00 | 26.17 | C |
| ATOM | 3075 | O | ILE | A | 712 | 14.196 | 30.543 | 50.687 | 1.00 | 26.14 | O |
| ATOM | 3076 | CB | ILE | A | 712 | 13.298 | 31.799 | 48.050 | 1.00 | 26.61 | C |
| ATOM | 3077 | CG1 | ILE | A | 712 | 12.251 | 32.041 | 46.961 | 1.00 | 26.21 | C |
| ATOM | 3078 | CG2 | ILE | A | 712 | 14.668 | 32.243 | 47.572 | 1.00 | 26.53 | C |
| ATOM | 3079 | CD1 | ILE | A | 712 | 11.979 | 33.494 | 46.696 | 1.00 | 26.70 | C |
| ATOM | 3080 | N | LEU | A | 713 | 15.407 | 29.343 | 49.211 | 1.00 | 25.71 | N |
| ATOM | 3081 | CA | LEU | A | 713 | 16.463 | 28.965 | 50.147 | 1.00 | 25.35 | C |
| ATOM | 3082 | C | LEU | A | 713 | 17.799 | 29.450 | 49.591 | 1.00 | 25.14 | C |
| ATOM | 3083 | O | LEU | A | 713 | 18.073 | 29.299 | 48.403 | 1.00 | 25.04 | O |
| ATOM | 3084 | CB | LEU | A | 713 | 16.462 | 27.440 | 50.332 | 1.00 | 25.27 | C |
| ATOM | 3085 | CG | LEU | A | 713 | 17.357 | 26.570 | 51.240 | 1.00 | 25.21 | C |
| ATOM | 3086 | CD1 | LEU | A | 713 | 18.864 | 26.793 | 51.065 | 1.00 | 24.85 | C |
| ATOM | 3087 | CD2 | LEU | A | 713 | 16.960 | 26.638 | 52.697 | 1.00 | 24.30 | C |
| ATOM | 3088 | N | SER | A | 714 | 18.625 | 30.037 | 50.448 | 1.00 | 25.00 | N |
| ATOM | 3089 | CA | SER | A | 714 | 19.942 | 30.498 | 50.029 | 1.00 | 24.85 | C |
| ATOM | 3090 | C | SER | A | 714 | 21.084 | 29.931 | 50.881 | 1.00 | 24.82 | C |
| ATOM | 3091 | O | SER | A | 714 | 21.061 | 30.021 | 52.113 | 1.00 | 24.66 | O |
| ATOM | 3092 | CB | SER | A | 714 | 19.988 | 32.026 | 50.013 | 1.00 | 24.72 | C |
| ATOM | 3093 | OG | SER | A | 714 | 21.243 | 32.478 | 49.542 | 1.00 | 24.67 | O |
| ATOM | 3094 | N | ASN | A | 715 | 22.077 | 29.352 | 50.211 | 1.00 | 24.85 | N |
| ATOM | 3095 | CA | ASN | A | 715 | 23.307 | 28.883 | 50.865 | 1.00 | 25.20 | C |
| ATOM | 3096 | C | ASN | A | 715 | 24.463 | 28.716 | 49.878 | 1.00 | 25.35 | C |
| ATOM | 3097 | O | ASN | A | 715 | 24.283 | 28.883 | 48.667 | 1.00 | 25.31 | O |
| ATOM | 3098 | CB | ASN | A | 715 | 23.076 | 27.578 | 51.653 | 1.00 | 24.93 | C |
| ATOM | 3099 | CG | ASN | A | 715 | 22.655 | 26.425 | 50.769 | 1.00 | 24.64 | C |
| ATOM | 3100 | OD1 | ASN | A | 715 | 22.284 | 26.620 | 49.612 | 1.00 | 24.79 | O |
| ATOM | 3101 | ND2 | ASN | A | 715 | 22.707 | 25.214 | 51.311 | 1.00 | 23.84 | N |
| ATOM | 3102 | N | ALA | A | 716 | 25.642 | 28.397 | 50.408 | 1.00 | 25.53 | N |
| ATOM | 3103 | CA | ALA | A | 716 | 26.821 | 28.133 | 49.586 | 1.00 | 25.91 | C |
| ATOM | 3104 | C | ALA | A | 716 | 26.618 | 26.909 | 48.701 | 1.00 | 26.22 | C |
| ATOM | 3105 | O | ALA | A | 716 | 26.096 | 25.888 | 49.149 | 1.00 | 26.23 | O |
| ATOM | 3106 | CB | ALA | A | 716 | 28.048 | 27.961 | 50.458 | 1.00 | 25.73 | C |
| ATOM | 3107 | N | GLY | A | 717 | 27.013 | 27.034 | 47.436 | 1.00 | 26.72 | N |
| ATOM | 3108 | CA | GLY | A | 717 | 26.961 | 25.925 | 46.485 | 1.00 | 27.20 | C |
| ATOM | 3109 | C | GLY | A | 717 | 28.117 | 24.967 | 46.691 | 1.00 | 27.74 | C |
| ATOM | 3110 | O | GLY | A | 717 | 27.986 | 23.764 | 46.458 | 1.00 | 27.73 | O |
| ATOM | 3111 | N | GLY | A | 718 | 29.247 | 25.502 | 47.142 | 1.00 | 28.24 | N |
| ATOM | 3112 | CA | GLY | A | 718 | 30.439 | 24.696 | 47.375 | 1.00 | 29.34 | C |
| ATOM | 3113 | C | GLY | A | 718 | 31.263 | 24.435 | 46.120 | 1.00 | 30.01 | C |
| ATOM | 3114 | O | GLY | A | 718 | 31.059 | 25.096 | 45.095 | 1.00 | 29.99 | O |
| ATOM | 3115 | N | PRO | A | 719 | 32.235 | 23.502 | 46.206 | 1.00 | 30.44 | N |
| ATOM | 3116 | CA | PRO | A | 719 | 32.637 | 22.833 | 47.446 | 1.00 | 30.70 | C |
| ATOM | 3117 | C | PRO | A | 719 | 33.302 | 23.868 | 48.342 | 1.00 | 31.04 | C |
| ATOM | 3118 | O | PRO | A | 719 | 33.976 | 24.767 | 47.830 | 1.00 | 31.59 | O |
| ATOM | 3119 | CB | PRO | A | 719 | 33.673 | 21.810 | 46.972 | 1.00 | 30.71 | C |
| ATOM | 3120 | CG | PRO | A | 719 | 34.221 | 22.385 | 45.709 | 1.00 | 30.60 | C |
| ATOM | 3121 | CD | PRO | A | 719 | 33.050 | 23.060 | 45.058 | 1.00 | 30.55 | C |
| ATOM | 3122 | N | LYS | A | 720 | 33.121 | 23.759 | 49.654 | 1.00 | 30.77 | N |
| ATOM | 3123 | CA | LYS | A | 720 | 33.574 | 24.810 | 50.577 | 1.00 | 30.85 | C |
| ATOM | 3124 | C | LYS | A | 720 | 32.602 | 26.000 | 50.587 | 1.00 | 30.50 | C |
| ATOM | 3125 | O | LYS | A | 720 | 31.617 | 26.024 | 49.841 | 1.00 | 30.39 | O |
| ATOM | 3126 | CB | LYS | A | 720 | 34.997 | 25.294 | 50.241 | 1.00 | 30.80 | C |
| ATOM | 3127 | CG | LYS | A | 720 | 36.109 | 24.270 | 50.450 | 1.00 | 31.45 | C |
| ATOM | 3128 | CD | LYS | A | 720 | 37.471 | 24.856 | 50.041 | 1.00 | 31.58 | C |
| ATOM | 3129 | CE | LYS | A | 720 | 38.634 | 24.181 | 50.774 | 1.00 | 32.90 | C |
| ATOM | 3130 | NZ | LYS | A | 720 | 38.837 | 22.749 | 50.378 | 1.00 | 32.69 | N |
| ATOM | 3131 | N | ARG | A | 721 | 32.889 | 26.988 | 51.433 | 1.00 | 30.16 | N |
| ATOM | 3132 | CA | ARG | A | 721 | 31.994 | 28.124 | 51.619 | 1.00 | 29.69 | C |
| ATOM | 3133 | C | ARG | A | 721 | 32.145 | 29.124 | 50.479 | 1.00 | 29.72 | C |
| ATOM | 3134 | O | ARG | A | 721 | 32.746 | 30.197 | 50.628 | 1.00 | 29.73 | O |
| ATOM | 3135 | CB | ARG | A | 721 | 32.199 | 28.761 | 52.997 | 1.00 | 29.60 | C |
| ATOM | 3136 | CG | ARG | A | 721 | 31.831 | 27.821 | 54.138 | 1.00 | 28.98 | C |
| ATOM | 3137 | CD | ARG | A | 721 | 32.058 | 28.449 | 55.498 | 1.00 | 28.72 | C |
| ATOM | 3138 | NE | ARG | A | 721 | 31.618 | 27.565 | 56.577 | 1.00 | 27.93 | N |
| ATOM | 3139 | CZ | ARG | A | 721 | 31.409 | 27.947 | 57.833 | 1.00 | 27.15 | C |
| ATOM | 3140 | NH1 | ARG | A | 721 | 31.599 | 29.209 | 58.194 | 1.00 | 27.91 | N |
| ATOM | 3141 | NH2 | ARG | A | 721 | 31.008 | 27.062 | 58.733 | 1.00 | 26.44 | N |
| ATOM | 3142 | N | GLU | A | 722 | 31.575 | 28.747 | 49.336 | 1.00 | 29.54 | N |
| ATOM | 3143 | CA | GLU | A | 722 | 31.720 | 29.491 | 48.096 | 1.00 | 29.56 | C |
| ATOM | 3144 | C | GLU | A | 722 | 30.567 | 29.198 | 47.136 | 1.00 | 29.27 | C |
| ATOM | 3145 | O | GLU | A | 722 | 29.791 | 28.259 | 47.351 | 1.00 | 28.91 | O |
| ATOM | 3146 | CB | GLU | A | 722 | 33.069 | 29.163 | 47.437 | 1.00 | 29.33 | C |
| ATOM | 3147 | CG | GLU | A | 722 | 33.116 | 27.813 | 46.726 | 1.00 | 29.98 | C |
| ATOM | 3148 | CD | GLU | A | 722 | 34.514 | 27.418 | 46.285 | 1.00 | 30.27 | C |
| ATOM | 3149 | OE1 | GLU | A | 722 | 35.348 | 28.313 | 46.029 | 1.00 | 31.99 | O |
| ATOM | 3150 | OE2 | GLU | A | 722 | 34.780 | 26.203 | 46.188 | 1.00 | 30.74 | O |
| ATOM | 3151 | N | ASN | A | 723 | 30.485 | 30.006 | 46.077 | 1.00 | 29.23 | N |
| ATOM | 3152 | CA | ASN | A | 723 | 29.463 | 29.899 | 45.022 | 1.00 | 29.42 | C |
| ATOM | 3153 | C | ASN | A | 723 | 28.032 | 30.004 | 45.547 | 1.00 | 29.03 | C |
| ATOM | 3154 | O | ASN | A | 723 | 27.261 | 29.054 | 45.463 | 1.00 | 28.86 | O |
| ATOM | 3155 | CB | ASN | A | 723 | 29.657 | 28.641 | 44.149 | 1.00 | 29.69 | C |
| ATOM | 3156 | CG | ASN | A | 723 | 29.013 | 28.778 | 42.763 | 1.00 | 31.10 | C |
| ATOM | 3157 | OD1 | ASN | A | 723 | 29.215 | 29.775 | 42.064 | 1.00 | 32.89 | O |
| ATOM | 3158 | ND2 | ASN | A | 723 | 28.244 | 27.769 | 42.361 | 1.00 | 32.07 | N |
| ATOM | 3159 | N | GLY | A | 724 | 27.695 | 31.175 | 46.081 | 1.00 | 28.87 | N |
| ATOM | 3160 | CA | GLY | A | 724 | 26.376 | 31.439 | 46.642 | 1.00 | 28.51 | C |
| ATOM | 3161 | C | GLY | A | 724 | 25.241 | 31.069 | 45.717 | 1.00 | 28.41 | C |
| ATOM | 3162 | O | GLY | A | 724 | 25.237 | 31.444 | 44.548 | 1.00 | 28.25 | O |
| ATOM | 3163 | N | MET | A | 725 | 24.280 | 30.324 | 46.254 | 1.00 | 28.44 | N |
| ATOM | 3164 | CA | MET | A | 725 | 23.150 | 29.829 | 45.478 | 1.00 | 28.50 | C |
| ATOM | 3165 | C | MET | A | 725 | 21.829 | 30.275 | 46.080 | 1.00 | 28.23 | C |
| ATOM | 3166 | O | MET | A | 725 | 21.680 | 30.347 | 47.301 | 1.00 | 28.16 | O |
| ATOM | 3167 | CB | MET | A | 725 | 23.171 | 28.299 | 45.410 | 1.00 | 28.52 | C |
| ATOM | 3168 | CG | MET | A | 725 | 24.381 | 27.707 | 44.723 | 1.00 | 29.50 | C |
| ATOM | 3169 | SD | MET | A | 725 | 24.497 | 28.173 | 42.991 | 1.00 | 31.56 | S |
| ATOM | 3170 | CE | MET | A | 725 | 23.597 | 26.826 | 42.225 | 1.00 | 32.01 | C |
| ATOM | 3171 | N | VAL | A | 726 | 20.876 | 30.581 | 45.209 | 1.00 | 27.97 | N |
| ATOM | 3172 | CA | VAL | A | 726 | 19.499 | 30.767 | 45.624 | 1.00 | 27.67 | C |
| ATOM | 3173 | C | VAL | A | 726 | 18.697 | 29.662 | 44.965 | 1.00 | 27.70 | C |
| ATOM | 3174 | O | VAL | A | 726 | 18.625 | 29.594 | 43.738 | 1.00 | 28.03 | O |
| ATOM | 3175 | CB | VAL | A | 726 | 18.953 | 32.146 | 45.228 | 1.00 | 27.71 | C |
| ATOM | 3176 | CG1 | VAL | A | 726 | 17.551 | 32.317 | 45.746 | 1.00 | 27.74 | C |
| ATOM | 3177 | CG2 | VAL | A | 726 | 19.838 | 33.257 | 45.793 | 1.00 | 27.29 | C |
| ATOM | 3178 | N | HIS | A | 727 | 18.126 | 28.785 | 45.788 | 1.00 | 27.40 | N |
| ATOM | 3179 | CA | HIS | A | 727 | 17.356 | 27.640 | 45.317 | 1.00 | 27.30 | C |
| ATOM | 3180 | C | HIS | A | 727 | 15.849 | 27.899 | 45.416 | 1.00 | 27.52 | C |
| ATOM | 3181 | O | HIS | A | 727 | 15.376 | 28.462 | 46.409 | 1.00 | 27.44 | O |
| ATOM | 3182 | CB | HIS | A | 727 | 17.707 | 26.392 | 46.127 | 1.00 | 27.23 | C |
| ATOM | 3183 | CG | HIS | A | 727 | 19.172 | 26.210 | 46.370 | 1.00 | 26.74 | C |
| ATOM | 3184 | ND1 | HIS | A | 727 | 19.992 | 25.523 | 45.501 | 1.00 | 26.22 | N |
| ATOM | 3185 | CD2 | HIS | A | 727 | 19.962 | 26.613 | 47.394 | 1.00 | 25.82 | C |
| ATOM | 3186 | CE1 | HIS | A | 727 | 21.223 | 25.513 | 45.977 | 1.00 | 26.18 | C |
| ATOM | 3187 | NE2 | HIS | A | 727 | 21.232 | 26.167 | 47.124 | 1.00 | 26.34 | N |
| ATOM | 3188 | N | LEU | A | 728 | 15.103 | 27.471 | 44.394 | 1.00 | 27.48 | N |
| ATOM | 3189 | CA | LEU | A | 728 | 13.656 | 27.672 | 44.345 | 1.00 | 27.77 | C |
| ATOM | 3190 | C | LEU | A | 728 | 12.913 | 26.341 | 44.351 | 1.00 | 28.04 | C |
| ATOM | 3191 | O | LEU | A | 728 | 13.161 | 25.488 | 43.507 | 1.00 | 27.99 | O |
| ATOM | 3192 | CB | LEU | A | 728 | 13.272 | 28.496 | 43.105 | 1.00 | 27.85 | C |
| ATOM | 3193 | CG | LEU | A | 728 | 11.793 | 28.690 | 42.727 | 1.00 | 27.49 | C |
| ATOM | 3194 | CD1 | LEU | A | 728 | 11.033 | 29.499 | 43.761 | 1.00 | 25.66 | C |
| ATOM | 3195 | CD2 | LEU | A | 728 | 11.681 | 29.350 | 41.358 | 1.00 | 27.66 | C |
| ATOM | 3196 | N | ALA | A | 729 | 12.009 | 26.168 | 45.310 | 1.00 | 28.51 | N |
| ATOM | 3197 | CA | ALA | A | 729 | 11.194 | 24.953 | 45.387 | 1.00 | 29.38 | C |
| ATOM | 3198 | C | ALA | A | 729 | 9.705 | 25.259 | 45.332 | 1.00 | 29.81 | C |
| ATOM | 3199 | O | ALA | A | 729 | 9.241 | 26.206 | 45.962 | 1.00 | 29.89 | O |
| ATOM | 3200 | CB | ALA | A | 729 | 11.509 | 24.169 | 46.648 | 1.00 | 29.16 | C |
| ATOM | 3201 | N | ARG | A | 730 | 8.960 | 24.456 | 44.579 | 1.00 | 30.58 | N |
| ATOM | 3202 | CA | ARG | A | 730 | 7.502 | 24.524 | 44.611 | 1.00 | 31.66 | C |
| ATOM | 3203 | C | ARG | A | 730 | 7.016 | 23.727 | 45.806 | 1.00 | 32.02 | C |
| ATOM | 3204 | O | ARG | A | 730 | 7.512 | 22.624 | 46.067 | 1.00 | 32.00 | O |
| ATOM | 3205 | CB | ARG | A | 730 | 6.893 | 23.966 | 43.318 | 1.00 | 31.51 | C |
| ATOM | 3206 | CG | ARG | A | 730 | 5.363 | 23.818 | 43.342 | 1.00 | 32.21 | C |
| ATOM | 3207 | CD | ARG | A | 730 | 4.786 | 23.385 | 41.986 | 1.00 | 32.63 | C |
| ATOM | 3208 | NE | ARG | A | 730 | 5.296 | 22.085 | 41.539 | 1.00 | 33.64 | N |
| ATOM | 3209 | CZ | ARG | A | 730 | 6.117 | 21.912 | 40.506 | 1.00 | 33.62 | C |
| ATOM | 3210 | NH1 | ARG | A | 730 | 6.522 | 22.952 | 39.790 | 1.00 | 33.72 | N |
| ATOM | 3211 | NH2 | ARG | A | 730 | 6.532 | 20.693 | 40.186 | 1.00 | 33.88 | N |
| ATOM | 3212 | N | VAL | A | 731 | 6.065 | 24.292 | 46.542 | 1.00 | 32.68 | N |
| ATOM | 3213 | CA | VAL | A | 731 | 5.385 | 23.541 | 47.586 | 1.00 | 33.65 | C |
| ATOM | 3214 | C | VAL | A | 731 | 4.257 | 22.735 | 46.947 | 1.00 | 34.53 | C |
| ATOM | 3215 | O | VAL | A | 731 | 3.318 | 23.301 | 46.383 | 1.00 | 34.33 | O |
| ATOM | 3216 | CB | VAL | A | 731 | 4.824 | 24.449 | 48.689 | 1.00 | 33.49 | C |
| ATOM | 3217 | CG1 | VAL | A | 731 | 4.184 | 23.611 | 49.790 | 1.00 | 33.14 | C |
| ATOM | 3218 | CG2 | VAL | A | 731 | 5.924 | 25.334 | 49.255 | 1.00 | 33.41 | C |
| ATOM | 3219 | N | GLU | A | 732 | 4.369 | 21.414 | 47.046 | 1.00 | 35.74 | N |
| ATOM | 3220 | CA | GLU | A | 732 | 3.415 | 20.500 | 46.433 | 1.00 | 37.09 | C |
| ATOM | 3221 | C | GLU | A | 732 | 2.117 | 20.330 | 47.217 | 1.00 | 38.22 | C |
| ATOM | 3222 | O | GLU | A | 732 | 1.159 | 21.085 | 47.032 | 1.00 | 38.53 | O |
| ATOM | 3223 | CB | GLU | A | 732 | 4.048 | 19.115 | 46.193 | 1.00 | 36.90 | C |
| ATOM | 3224 | CG | GLU | A | 732 | 5.187 | 19.072 | 45.181 | 1.00 | 36.31 | C |
| ATOM | 3225 | CD | GLU | A | 732 | 4.789 | 19.550 | 43.791 | 1.00 | 35.68 | C |
| ATOM | 3226 | OE1 | GLU | A | 732 | 3.578 | 19.609 | 43.483 | 1.00 | 34.90 | O |
| ATOM | 3227 | OE2 | GLU | A | 732 | 5.703 | 19.865 | 43.004 | 1.00 | 35.40 | O |
| ATOM | 3228 | N | GLU | A | 733 | 2.100 | 19.330 | 48.093 | 1.00 | 39.63 | N |
| ATOM | 3229 | CA | GLU | A | 733 | 0.854 | 18.702 | 48.512 | 1.00 | 40.84 | C |
| ATOM | 3230 | C | GLU | A | 733 | 0.825 | 18.350 | 49.994 | 1.00 | 41.32 | C |
| ATOM | 3231 | O | GLU | A | 733 | −0.004 | 18.881 | 50.734 | 1.00 | 41.78 | O |
| ATOM | 3232 | CB | GLU | A | 733 | 0.604 | 17.447 | 47.657 | 1.00 | 41.08 | C |
| ATOM | 3233 | CG | GLU | A | 733 | 1.665 | 16.355 | 47.838 | 1.00 | 42.03 | C |
| ATOM | 3234 | CD | GLU | A | 733 | 1.976 | 15.606 | 46.564 | 1.00 | 43.87 | C |
| ATOM | 3235 | OE1 | GLU | A | 733 | 1.126 | 14.806 | 46.116 | 1.00 | 45.07 | O |
| ATOM | 3236 | OE2 | GLU | A | 733 | 3.083 | 15.802 | 46.017 | 1.00 | 44.28 | O |
| ATOM | 3237 | N | ASN | A | 734 | 1.734 | 17.473 | 50.426 | 1.00 | 41.94 | N |
| ATOM | 3238 | CA | ASN | A | 734 | 1.642 | 16.859 | 51.763 | 1.00 | 42.57 | C |
| ATOM | 3239 | C | ASN | A | 734 | 2.939 | 16.672 | 52.584 | 1.00 | 42.56 | C |
| ATOM | 3240 | O | ASN | A | 734 | 3.195 | 15.570 | 53.085 | 1.00 | 42.70 | O |
| ATOM | 3241 | CB | ASN | A | 734 | 0.906 | 15.509 | 51.670 | 1.00 | 42.74 | C |
| ATOM | 3242 | CG | ASN | A | 734 | −0.504 | 15.564 | 52.237 | 1.00 | 43.60 | C |
| ATOM | 3243 | OD1 | ASN | A | 734 | −0.730 | 16.072 | 53.341 | 1.00 | 44.32 | O |
| ATOM | 3244 | ND2 | ASN | A | 734 | −1.460 | 15.016 | 51.489 | 1.00 | 43.91 | N |
| ATOM | 3245 | N | GLY | A | 735 | 3.761 | 17.711 | 52.736 | 1.00 | 42.53 | N |
| ATOM | 3246 | CA | GLY | A | 735 | 3.669 | 18.979 | 52.012 | 1.00 | 42.26 | C |
| ATOM | 3247 | C | GLY | A | 735 | 5.000 | 19.056 | 51.303 | 1.00 | 42.05 | C |
| ATOM | 3248 | O | GLY | A | 735 | 5.971 | 19.608 | 51.830 | 1.00 | 42.01 | O |
| ATOM | 3249 | N | GLU | A | 736 | 5.049 | 18.465 | 50.116 | 1.00 | 41.76 | N |
| ATOM | 3250 | CA | GLU | A | 736 | 6.324 | 18.130 | 49.490 | 1.00 | 41.38 | C |
| ATOM | 3251 | C | GLU | A | 736 | 6.964 | 19.291 | 48.733 | 1.00 | 40.68 | C |
| ATOM | 3252 | O | GLU | A | 736 | 6.293 | 20.260 | 48.356 | 1.00 | 40.59 | O |
| ATOM | 3253 | CB | GLU | A | 736 | 6.172 | 16.886 | 48.610 | 1.00 | 41.61 | C |
| ATOM | 3254 | CG | GLU | A | 736 | 5.622 | 15.691 | 49.387 | 1.00 | 42.97 | C |
| ATOM | 3255 | CD | GLU | A | 736 | 5.690 | 14.386 | 48.621 | 1.00 | 44.95 | C |
| ATOM | 3256 | OE1 | GLU | A | 736 | 6.402 | 14.321 | 47.592 | 1.00 | 45.28 | O |
| ATOM | 3257 | OE2 | GLU | A | 736 | 5.024 | 13.416 | 49.059 | 1.00 | 46.05 | O |
| ATOM | 3258 | N | LEU | A | 737 | 8.275 | 19.183 | 48.533 | 1.00 | 39.73 | N |
| ATOM | 3259 | CA | LEU | A | 737 | 9.046 | 20.227 | 47.882 | 1.00 | 38.85 | C |
| ATOM | 3260 | C | LEU | A | 737 | 9.680 | 19.709 | 46.599 | 1.00 | 38.35 | C |
| ATOM | 3261 | O | LEU | A | 737 | 10.304 | 18.647 | 46.589 | 1.00 | 38.38 | O |
| ATOM | 3262 | CB | LEU | A | 737 | 10.119 | 20.766 | 48.832 | 1.00 | 38.82 | C |
| ATOM | 3263 | CG | LEU | A | 737 | 9.661 | 21.231 | 50.220 | 1.00 | 38.66 | C |
| ATOM | 3264 | CD1 | LEU | A | 737 | 10.852 | 21.421 | 51.135 | 1.00 | 38.55 | C |
| ATOM | 3265 | CD2 | LEU | A | 737 | 8.829 | 22.510 | 50.138 | 1.00 | 38.52 | C |
| ATOM | 3266 | N | THR | A | 738 | 9.504 | 20.460 | 45.518 | 1.00 | 37.55 | N |
| ATOM | 3267 | CA | THR | A | 738 | 10.124 | 20.129 | 44.246 | 1.00 | 36.79 | C |
| ATOM | 3268 | C | THR | A | 738 | 11.096 | 21.234 | 43.876 | 1.00 | 36.40 | C |
| ATOM | 3269 | O | THR | A | 738 | 10.691 | 22.360 | 43.583 | 1.00 | 36.39 | O |
| ATOM | 3270 | CB | THR | A | 738 | 9.078 | 19.937 | 43.131 | 1.00 | 36.71 | C |
| ATOM | 3271 | OG1 | THR | A | 738 | 8.106 | 18.974 | 43.552 | 1.00 | 36.46 | O |
| ATOM | 3272 | CG2 | THR | A | 738 | 9.734 | 19.446 | 41.852 | 1.00 | 36.74 | C |
| ATOM | 3273 | N | TRP | A | 739 | 12.380 | 20.898 | 43.893 | 1.00 | 35.93 | N |
| ATOM | 3274 | CA | TRP | A | 739 | 13.438 | 21.852 | 43.585 | 1.00 | 35.44 | C |
| ATOM | 3275 | C | TRP | A | 739 | 13.505 | 22.125 | 42.098 | 1.00 | 35.52 | C |
| ATOM | 3276 | O | TRP | A | 739 | 13.827 | 21.239 | 41.310 | 1.00 | 35.58 | O |
| ATOM | 3277 | CB | TRP | A | 739 | 14.779 | 21.371 | 44.147 | 1.00 | 35.01 | C |
| ATOM | 3278 | CG | TRP | A | 739 | 14.752 | 21.381 | 45.630 | 1.00 | 34.52 | C |
| ATOM | 3279 | CD1 | TRP | A | 739 | 14.635 | 20.304 | 46.453 | 1.00 | 34.23 | C |
| ATOM | 3280 | CD2 | TRP | A | 739 | 14.773 | 22.537 | 46.477 | 1.00 | 34.30 | C |
| ATOM | 3281 | NE1 | TRP | A | 739 | 14.608 | 20.713 | 47.764 | 1.00 | 34.23 | N |
| ATOM | 3282 | CE2 | TRP | A | 739 | 14.690 | 22.079 | 47.806 | 1.00 | 34.00 | C |
| ATOM | 3283 | CE3 | TRP | A | 739 | 14.871 | 23.916 | 46.241 | 1.00 | 34.29 | C |
| ATOM | 3284 | CZ2 | TRP | A | 739 | 14.703 | 22.948 | 48.897 | 1.00 | 34.13 | C |
| ATOM | 3285 | CZ3 | TRP | A | 739 | 14.876 | 24.779 | 47.326 | 1.00 | 34.36 | C |
| ATOM | 3286 | CH2 | TRP | A | 739 | 14.797 | 24.291 | 48.639 | 1.00 | 34.36 | C |
| ATOM | 3287 | N | LEU | A | 740 | 13.194 | 23.367 | 41.733 | 1.00 | 35.72 | N |
| ATOM | 3288 | CA | LEU | A | 740 | 12.974 | 23.742 | 40.340 | 1.00 | 35.84 | C |
| ATOM | 3289 | C | LEU | A | 740 | 14.180 | 24.390 | 39.683 | 1.00 | 36.05 | C |
| ATOM | 3290 | O | LEU | A | 740 | 14.465 | 24.130 | 38.510 | 1.00 | 35.95 | O |
| ATOM | 3291 | CB | LEU | A | 740 | 11.747 | 24.648 | 40.217 | 1.00 | 35.90 | C |
| ATOM | 3292 | CG | LEU | A | 740 | 10.375 | 24.007 | 40.461 | 1.00 | 36.16 | C |
| ATOM | 3293 | CD1 | LEU | A | 740 | 9.303 | 25.082 | 40.577 | 1.00 | 36.47 | C |
| ATOM | 3294 | CD2 | LEU | A | 740 | 10.013 | 22.989 | 39.368 | 1.00 | 36.32 | C |
| ATOM | 3295 | N | LYS | A | 741 | 14.878 | 25.247 | 40.424 | 1.00 | 36.22 | N |
| ATOM | 3296 | CA | LYS | A | 741 | 16.068 | 25.890 | 39.882 | 1.00 | 36.32 | C |
| ATOM | 3297 | C | LYS | A | 741 | 17.078 | 26.300 | 40.945 | 1.00 | 35.92 | C |
| ATOM | 3298 | O | LYS | A | 741 | 16.724 | 26.541 | 42.105 | 1.00 | 35.96 | O |
| ATOM | 3299 | CB | LYS | A | 741 | 15.697 | 27.073 | 38.981 | 1.00 | 36.61 | C |
| ATOM | 3300 | CG | LYS | A | 741 | 16.420 | 27.018 | 37.644 | 1.00 | 38.19 | C |
| ATOM | 3301 | CD | LYS | A | 741 | 16.146 | 28.235 | 36.800 | 1.00 | 41.04 | C |
| ATOM | 3302 | CE | LYS | A | 741 | 17.165 | 28.347 | 35.679 | 1.00 | 42.69 | C |
| ATOM | 3303 | NZ | LYS | A | 741 | 17.545 | 29.779 | 35.497 | 1.00 | 44.09 | N |
| ATOM | 3304 | N | HIS | A | 742 | 18.340 | 26.369 | 40.524 | 1.00 | 35.48 | N |
| ATOM | 3305 | CA | HIS | A | 742 | 19.465 | 26.629 | 41.411 | 1.00 | 34.81 | C |
| ATOM | 3306 | C | HIS | A | 742 | 20.356 | 27.676 | 40.759 | 1.00 | 34.51 | C |
| ATOM | 3307 | O | HIS | A | 742 | 21.215 | 27.346 | 39.941 | 1.00 | 34.81 | O |
| ATOM | 3308 | CB | HIS | A | 742 | 20.243 | 25.332 | 41.672 | 1.00 | 34.72 | C |
| ATOM | 3309 | CG | HIS | A | 742 | 19.367 | 24.152 | 41.963 | 1.00 | 34.17 | C |
| ATOM | 3310 | ND1 | HIS | A | 742 | 18.881 | 23.878 | 43.224 | 1.00 | 34.17 | N |
| ATOM | 3311 | CD2 | HIS | A | 742 | 18.876 | 23.184 | 41.153 | 1.00 | 33.89 | C |
| ATOM | 3312 | CE1 | HIS | A | 742 | 18.135 | 22.788 | 43.181 | 1.00 | 33.97 | C |
| ATOM | 3313 | NE2 | HIS | A | 742 | 18.112 | 22.348 | 41.935 | 1.00 | 34.13 | N |
| ATOM | 3314 | N | ASN | A | 743 | 20.144 | 28.939 | 41.124 | 1.00 | 33.99 | N |
| ATOM | 3315 | CA | ASN | A | 743 | 20.777 | 30.067 | 40.441 | 1.00 | 33.52 | C |
| ATOM | 3316 | C | ASN | A | 743 | 21.896 | 30.722 | 41.245 | 1.00 | 32.97 | C |
| ATOM | 3317 | O | ASN | A | 743 | 21.674 | 31.112 | 42.394 | 1.00 | 33.20 | O |
| ATOM | 3318 | CB | ASN | A | 743 | 19.724 | 31.117 | 40.075 | 1.00 | 33.64 | C |
| ATOM | 3319 | CG | ASN | A | 743 | 18.794 | 30.655 | 38.968 | 1.00 | 34.30 | C |
| ATOM | 3320 | OD1 | ASN | A | 743 | 19.239 | 30.182 | 37.916 | 1.00 | 34.90 | O |
| ATOM | 3321 | ND2 | ASN | A | 743 | 17.491 | 30.800 | 39.194 | 1.00 | 34.91 | N |
| ATOM | 3322 | N | PRO | A | 744 | 23.098 | 30.860 | 40.640 | 1.00 | 32.26 | N |
| ATOM | 3323 | CA | PRO | A | 744 | 24.236 | 31.475 | 41.326 | 1.00 | 31.63 | C |
| ATOM | 3324 | C | PRO | A | 744 | 23.997 | 32.959 | 41.570 | 1.00 | 31.15 | C |
| ATOM | 3325 | O | PRO | A | 744 | 23.542 | 33.667 | 40.676 | 1.00 | 31.05 | O |
| ATOM | 3326 | CB | PRO | A | 744 | 25.405 | 31.278 | 40.342 | 1.00 | 31.83 | C |
| ATOM | 3327 | CG | PRO | A | 744 | 24.931 | 30.274 | 39.337 | 1.00 | 31.74 | C |
| ATOM | 3328 | CD | PRO | A | 744 | 23.448 | 30.451 | 39.266 | 1.00 | 32.17 | C |
| ATOM | 3329 | N | ILE | A | 745 | 24.294 | 33.417 | 42.782 | 1.00 | 30.81 | N |
| ATOM | 3330 | CA | ILE | A | 745 | 24.078 | 34.811 | 43.151 | 1.00 | 30.35 | C |
| ATOM | 3331 | C | ILE | A | 745 | 25.383 | 35.532 | 43.509 | 1.00 | 30.17 | C |
| ATOM | 3332 | O | ILE | A | 745 | 25.473 | 36.757 | 43.402 | 1.00 | 29.97 | O |
| ATOM | 3333 | CB | ILE | A | 745 | 23.007 | 34.950 | 44.277 | 1.00 | 30.41 | C |
| ATOM | 3334 | CG1 | ILE | A | 745 | 22.519 | 36.396 | 44.381 | 1.00 | 29.88 | C |
| ATOM | 3335 | CG2 | ILE | A | 745 | 23.524 | 34.414 | 45.624 | 1.00 | 30.54 | C |
| ATOM | 3336 | CD1 | ILE | A | 745 | 21.113 | 36.523 | 44.841 | 1.00 | 29.70 | C |
| ATOM | 3337 | N | GLN | A | 746 | 26.387 | 34.763 | 43.926 | 1.00 | 29.93 | N |
| ATOM | 3338 | CA | GLN | A | 746 | 27.687 | 35.319 | 44.273 | 1.00 | 29.60 | C |
| ATOM | 3339 | C | GLN | A | 746 | 28.801 | 34.293 | 44.111 | 1.00 | 29.73 | C |
| ATOM | 3340 | O | GLN | A | 746 | 28.825 | 33.265 | 44.799 | 1.00 | 29.82 | O |
| ATOM | 3341 | CB | GLN | A | 746 | 27.674 | 35.882 | 45.699 | 1.00 | 29.39 | C |
| ATOM | 3342 | CG | GLN | A | 746 | 28.993 | 36.493 | 46.142 | 1.00 | 29.02 | C |
| ATOM | 3343 | CD | GLN | A | 746 | 29.435 | 37.643 | 45.252 | 1.00 | 28.60 | C |
| ATOM | 3344 | OE1 | GLN | A | 746 | 28.836 | 38.721 | 45.267 | 1.00 | 28.66 | O |
| ATOM | 3345 | NE2 | GLN | A | 746 | 30.486 | 37.419 | 44.473 | 1.00 | 26.94 | N |
| ATOM | 3346 | N | LYS | A | 747 | 29.715 | 34.584 | 43.191 | 1.00 | 29.76 | N |
| ATOM | 3347 | CA | LYS | A | 747 | 30.919 | 33.791 | 42.999 | 1.00 | 29.99 | C |
| ATOM | 3348 | C | LYS | A | 747 | 31.947 | 34.170 | 44.059 | 1.00 | 29.68 | C |
| ATOM | 3349 | O | LYS | A | 747 | 31.830 | 35.218 | 44.691 | 1.00 | 29.60 | O |
| ATOM | 3350 | CB | LYS | A | 747 | 31.495 | 34.035 | 41.604 | 1.00 | 30.35 | C |
| ATOM | 3351 | CG | LYS | A | 747 | 30.527 | 33.715 | 40.475 | 1.00 | 31.96 | C |
| ATOM | 3352 | CD | LYS | A | 747 | 31.024 | 34.293 | 39.156 | 1.00 | 34.52 | C |
| ATOM | 3353 | CE | LYS | A | 747 | 29.860 | 34.748 | 38.273 | 1.00 | 35.36 | C |
| ATOM | 3354 | NZ | LYS | A | 747 | 30.345 | 35.313 | 36.979 | 1.00 | 35.56 | N |
| ATOM | 3355 | N | GLY | A | 748 | 32.956 | 33.320 | 44.241 | 1.00 | 29.43 | N |
| ATOM | 3356 | CA | GLY | A | 748 | 33.984 | 33.542 | 45.259 | 1.00 | 28.77 | C |
| ATOM | 3357 | C | GLY | A | 748 | 33.491 | 33.084 | 46.618 | 1.00 | 28.41 | C |
| ATOM | 3358 | O | GLY | A | 748 | 32.558 | 32.286 | 46.699 | 1.00 | 28.29 | O |
| ATOM | 3359 | N | GLU | A | 749 | 34.113 | 33.590 | 47.681 | 1.00 | 28.06 | N |
| ATOM | 3360 | CA | GLU | A | 749 | 33.739 | 33.233 | 49.051 | 1.00 | 28.06 | C |
| ATOM | 3361 | C | GLU | A | 749 | 32.268 | 33.541 | 49.355 | 1.00 | 27.51 | C |
| ATOM | 3362 | O | GLU | A | 749 | 31.776 | 34.618 | 49.028 | 1.00 | 27.33 | O |
| ATOM | 3363 | CB | GLU | A | 749 | 34.646 | 33.955 | 50.055 | 1.00 | 28.41 | C |
| ATOM | 3364 | CG | GLU | A | 749 | 36.027 | 33.322 | 50.235 | 1.00 | 30.01 | C |
| ATOM | 3365 | CD | GLU | A | 749 | 35.970 | 31.954 | 50.899 | 1.00 | 32.99 | C |
| ATOM | 3366 | OE1 | GLU | A | 749 | 35.064 | 31.726 | 51.743 | 1.00 | 35.42 | O |
| ATOM | 3367 | OE2 | GLU | A | 749 | 36.829 | 31.102 | 50.580 | 1.00 | 33.53 | O |
| ATOM | 3368 | N | PHE | A | 750 | 31.569 | 32.587 | 49.967 | 1.00 | 26.86 | N |
| ATOM | 3369 | CA | PHE | A | 750 | 30.167 | 32.787 | 50.336 | 1.00 | 26.31 | C |
| ATOM | 3370 | C | PHE | A | 750 | 29.764 | 31.968 | 51.555 | 1.00 | 26.04 | C |
| ATOM | 3371 | O | PHE | A | 750 | 29.811 | 30.740 | 51.524 | 1.00 | 26.24 | O |
| ATOM | 3372 | CB | PHE | A | 750 | 29.244 | 32.439 | 49.163 | 1.00 | 26.12 | C |
| ATOM | 3373 | CG | PHE | A | 750 | 27.804 | 32.806 | 49.394 | 1.00 | 25.77 | C |
| ATOM | 3374 | CD1 | PHE | A | 750 | 27.311 | 34.034 | 48.960 | 1.00 | 24.39 | C |
| ATOM | 3375 | CD2 | PHE | A | 750 | 26.939 | 31.926 | 50.049 | 1.00 | 25.14 | C |
| ATOM | 3376 | CE1 | PHE | A | 750 | 25.980 | 34.371 | 49.163 | 1.00 | 24.41 | C |
| ATOM | 3377 | CE2 | PHE | A | 750 | 25.606 | 32.260 | 50.257 | 1.00 | 24.53 | C |
| ATOM | 3378 | CZ | PHE | A | 750 | 25.125 | 33.484 | 49.814 | 1.00 | 24.30 | C |
| ATOM | 3379 | N | ALA | A | 751 | 29.342 | 32.645 | 52.616 | 1.00 | 25.60 | N |
| ATOM | 3380 | CA | ALA | A | 751 | 28.938 | 31.948 | 53.835 | 1.00 | 25.29 | C |
| ATOM | 3381 | C | ALA | A | 751 | 27.509 | 32.316 | 54.269 | 1.00 | 24.92 | C |
| ATOM | 3382 | O | ALA | A | 751 | 26.566 | 32.089 | 53.515 | 1.00 | 25.03 | O |
| ATOM | 3383 | CB | ALA | A | 751 | 29.963 | 32.173 | 54.951 | 1.00 | 25.27 | C |
| ATOM | 3384 | N | TYR | A | 752 | 27.351 | 32.896 | 55.456 | 1.00 | 24.51 | N |
| ATOM | 3385 | CA | TYR | A | 752 | 26.027 | 33.181 | 56.013 | 1.00 | 24.29 | C |
| ATOM | 3386 | C | TYR | A | 752 | 25.229 | 34.163 | 55.170 | 1.00 | 24.28 | C |
| ATOM | 3387 | O | TYR | A | 752 | 25.799 | 34.934 | 54.397 | 1.00 | 24.50 | O |
| ATOM | 3388 | CB | TYR | A | 752 | 26.143 | 33.702 | 57.445 | 1.00 | 24.20 | C |
| ATOM | 3389 | CG | TYR | A | 752 | 26.530 | 32.663 | 58.476 | 1.00 | 23.96 | C |
| ATOM | 3390 | CD1 | TYR | A | 752 | 26.060 | 32.753 | 59.783 | 1.00 | 23.48 | C |
| ATOM | 3391 | CD2 | TYR | A | 752 | 27.365 | 31.588 | 58.146 | 1.00 | 23.60 | C |
| ATOM | 3392 | CE1 | TYR | A | 752 | 26.414 | 31.811 | 60.738 | 1.00 | 24.01 | C |
| ATOM | 3393 | CE2 | TYR | A | 752 | 27.725 | 30.640 | 59.092 | 1.00 | 23.59 | C |
| ATOM | 3394 | CZ | TYR | A | 752 | 27.245 | 30.757 | 60.385 | 1.00 | 24.10 | C |
| ATOM | 3395 | OH | TYR | A | 752 | 27.591 | 29.817 | 61.325 | 1.00 | 24.41 | O |
| ATOM | 3396 | N | ASN | A | 753 | 23.908 | 34.131 | 55.329 | 1.00 | 24.12 | N |
| ATOM | 3397 | CA | ASN | A | 753 | 23.001 | 34.935 | 54.509 | 1.00 | 23.99 | C |
| ATOM | 3398 | C | ASN | A | 753 | 21.586 | 34.980 | 55.073 | 1.00 | 23.83 | C |
| ATOM | 3399 | O | ASN | A | 753 | 21.189 | 34.101 | 55.844 | 1.00 | 23.40 | O |
| ATOM | 3400 | CB | ASN | A | 753 | 22.963 | 34.406 | 53.068 | 1.00 | 23.96 | C |
| ATOM | 3401 | CG | ASN | A | 753 | 22.316 | 33.044 | 52.968 | 1.00 | 23.99 | C |
| ATOM | 3402 | OD1 | ASN | A | 753 | 21.088 | 32.928 | 52.934 | 1.00 | 23.59 | O |
| ATOM | 3403 | ND2 | ASN | A | 753 | 23.141 | 31.997 | 52.920 | 1.00 | 23.78 | N |
| ATOM | 3404 | N | SER | A | 754 | 20.830 | 36.000 | 54.663 | 1.00 | 23.78 | N |
| ATOM | 3405 | CA | SER | A | 754 | 19.449 | 36.182 | 55.110 | 1.00 | 23.84 | C |
| ATOM | 3406 | C | SER | A | 754 | 18.556 | 36.746 | 54.001 | 1.00 | 23.94 | C |
| ATOM | 3407 | O | SER | A | 754 | 18.852 | 37.794 | 53.415 | 1.00 | 23.85 | O |
| ATOM | 3408 | CB | SER | A | 754 | 19.395 | 37.074 | 56.351 | 1.00 | 23.70 | C |
| ATOM | 3409 | OG | SER | A | 754 | 18.113 | 37.042 | 56.951 | 1.00 | 23.74 | O |
| ATOM | 3410 | N | LEU | A | 755 | 17.468 | 36.033 | 53.722 | 1.00 | 24.16 | N |
| ATOM | 3411 | CA | LEU | A | 755 | 16.509 | 36.426 | 52.692 | 1.00 | 24.50 | C |
| ATOM | 3412 | C | LEU | A | 755 | 15.300 | 37.159 | 53.264 | 1.00 | 24.79 | C |
| ATOM | 3413 | O | LEU | A | 755 | 14.854 | 36.875 | 54.376 | 1.00 | 24.62 | O |
| ATOM | 3414 | CB | LEU | A | 755 | 16.011 | 35.194 | 51.938 | 1.00 | 24.33 | C |
| ATOM | 3415 | CG | LEU | A | 755 | 16.972 | 34.364 | 51.094 | 1.00 | 24.06 | C |
| ATOM | 3416 | CD1 | LEU | A | 755 | 16.331 | 33.016 | 50.817 | 1.00 | 23.29 | C |
| ATOM | 3417 | CD2 | LEU | A | 755 | 17.339 | 35.076 | 49.794 | 1.00 | 22.82 | C |
| ATOM | 3418 | N | GLN | A | 756 | 14.773 | 38.098 | 52.485 | 1.00 | 25.28 | N |
| ATOM | 3419 | CA | GLN | A | 756 | 13.483 | 38.711 | 52.779 | 1.00 | 25.79 | C |
| ATOM | 3420 | C | GLN | A | 756 | 12.692 | 38.940 | 51.507 | 1.00 | 26.50 | C |
| ATOM | 3421 | O | GLN | A | 756 | 13.257 | 39.130 | 50.424 | 1.00 | 26.40 | O |
| ATOM | 3422 | CB | GLN | A | 756 | 13.635 | 40.037 | 53.533 | 1.00 | 25.58 | C |
| ATOM | 3423 | CG | GLN | A | 756 | 13.944 | 39.899 | 55.019 | 1.00 | 24.84 | C |
| ATOM | 3424 | CD | GLN | A | 756 | 12.816 | 39.270 | 55.819 | 1.00 | 23.88 | C |
| ATOM | 3425 | OE1 | GLN | A | 756 | 11.903 | 39.963 | 56.268 | 1.00 | 23.82 | O |
| ATOM | 3426 | NE2 | GLN | A | 756 | 12.890 | 37.954 | 56.023 | 1.00 | 21.62 | N |
| ATOM | 3427 | N | GLU | A | 757 | 11.374 | 38.895 | 51.660 | 1.00 | 27.24 | N |
| ATOM | 3428 | CA | GLU | A | 757 | 10.438 | 39.295 | 50.631 | 1.00 | 28.11 | C |
| ATOM | 3429 | C | GLU | A | 757 | 10.424 | 40.817 | 50.655 | 1.00 | 28.21 | C |
| ATOM | 3430 | O | GLU | A | 757 | 10.441 | 41.419 | 51.729 | 1.00 | 28.26 | O |
| ATOM | 3431 | CB | GLU | A | 757 | 9.060 | 38.755 | 50.991 | 1.00 | 28.00 | C |
| ATOM | 3432 | CG | GLU | A | 757 | 8.121 | 38.508 | 49.826 | 1.00 | 29.19 | C |
| ATOM | 3433 | CD | GLU | A | 757 | 6.898 | 37.698 | 50.242 | 1.00 | 29.26 | C |
| ATOM | 3434 | OE1 | GLU | A | 757 | 6.560 | 37.703 | 51.447 | 1.00 | 31.25 | O |
| ATOM | 3435 | OE2 | GLU | A | 757 | 6.271 | 37.058 | 49.370 | 1.00 | 31.40 | O |
| ATOM | 3436 | N | LEU | A | 758 | 10.408 | 41.441 | 49.482 | 1.00 | 28.62 | N |
| ATOM | 3437 | CA | LEU | A | 758 | 10.467 | 42.899 | 49.404 | 1.00 | 28.96 | C |
| ATOM | 3438 | C | LEU | A | 758 | 9.153 | 43.552 | 48.991 | 1.00 | 29.28 | C |
| ATOM | 3439 | O | LEU | A | 758 | 8.974 | 44.757 | 49.165 | 1.00 | 29.40 | O |
| ATOM | 3440 | CB | LEU | A | 758 | 11.602 | 43.344 | 48.477 | 1.00 | 28.80 | C |
| ATOM | 3441 | CG | LEU | A | 758 | 12.901 | 43.843 | 49.120 | 1.00 | 29.29 | C |
| ATOM | 3442 | CD1 | LEU | A | 758 | 13.366 | 42.977 | 50.293 | 1.00 | 28.64 | C |
| ATOM | 3443 | CD2 | LEU | A | 758 | 13.988 | 43.953 | 48.064 | 1.00 | 28.93 | C |
| ATOM | 3444 | N | GLY | A | 759 | 8.232 | 42.751 | 48.468 | 1.00 | 29.68 | N |
| ATOM | 3445 | CA | GLY | A | 759 | 7.006 | 43.272 | 47.890 | 1.00 | 30.12 | C |
| ATOM | 3446 | C | GLY | A | 759 | 7.180 | 43.406 | 46.392 | 1.00 | 30.55 | C |
| ATOM | 3447 | O | GLY | A | 759 | 8.305 | 43.507 | 45.895 | 1.00 | 30.45 | O |
| ATOM | 3448 | N | ASN | A | 760 | 6.062 | 43.371 | 45.672 | 1.00 | 30.78 | N |
| ATOM | 3449 | CA | ASN | A | 760 | 6.054 | 43.572 | 44.224 | 1.00 | 30.87 | C |
| ATOM | 3450 | C | ASN | A | 760 | 6.916 | 42.592 | 43.426 | 1.00 | 30.84 | C |
| ATOM | 3451 | O | ASN | A | 760 | 7.468 | 42.956 | 42.387 | 1.00 | 31.30 | O |
| ATOM | 3452 | CB | ASN | A | 760 | 6.444 | 45.016 | 43.889 | 1.00 | 30.82 | C |
| ATOM | 3453 | CG | ASN | A | 760 | 5.573 | 46.037 | 44.595 | 1.00 | 31.00 | C |
| ATOM | 3454 | OD1 | ASN | A | 760 | 4.348 | 45.901 | 44.650 | 1.00 | 30.22 | O |
| ATOM | 3455 | ND2 | ASN | A | 760 | 6.203 | 47.078 | 45.131 | 1.00 | 31.07 | N |
| ATOM | 3456 | N | GLY | A | 761 | 7.017 | 41.354 | 43.903 | 1.00 | 30.83 | N |
| ATOM | 3457 | CA | GLY | A | 761 | 7.779 | 40.307 | 43.211 | 1.00 | 30.33 | C |
| ATOM | 3458 | C | GLY | A | 761 | 9.293 | 40.464 | 43.279 | 1.00 | 30.11 | C |
| ATOM | 3459 | O | GLY | A | 761 | 10.017 | 39.864 | 42.481 | 1.00 | 30.25 | O |
| ATOM | 3460 | N | GLU | A | 762 | 9.765 | 41.273 | 44.227 | 1.00 | 29.67 | N |
| ATOM | 3461 | CA | GLU | A | 762 | 11.194 | 41.473 | 44.464 | 1.00 | 29.39 | C |
| ATOM | 3462 | C | GLU | A | 762 | 11.620 | 40.872 | 45.796 | 1.00 | 28.69 | C |
| ATOM | 3463 | O | GLU | A | 762 | 10.803 | 40.697 | 46.701 | 1.00 | 28.61 | O |
| ATOM | 3464 | CB | GLU | A | 762 | 11.564 | 42.962 | 44.425 | 1.00 | 29.53 | C |
| ATOM | 3465 | CG | GLU | A | 762 | 11.362 | 43.622 | 43.061 | 1.00 | 29.98 | C |
| ATOM | 3466 | CD | GLU | A | 762 | 12.089 | 44.953 | 42.913 | 1.00 | 30.13 | C |
| ATOM | 3467 | OE1 | GLU | A | 762 | 12.521 | 45.544 | 43.928 | 1.00 | 30.44 | O |
| ATOM | 3468 | OE2 | GLU | A | 762 | 12.223 | 45.417 | 41.759 | 1.00 | 31.91 | O |
| ATOM | 3469 | N | TYR | A | 763 | 12.909 | 40.557 | 45.901 | 1.00 | 28.18 | N |
| ATOM | 3470 | CA | TYR | A | 763 | 13.475 | 39.930 | 47.088 | 1.00 | 27.55 | C |
| ATOM | 3471 | C | TYR | A | 763 | 14.837 | 40.519 | 47.420 | 1.00 | 27.34 | C |
| ATOM | 3472 | O | TYR | A | 763 | 15.532 | 41.029 | 46.540 | 1.00 | 27.23 | O |
| ATOM | 3473 | CB | TYR | A | 763 | 13.603 | 38.421 | 46.885 | 1.00 | 27.43 | C |
| ATOM | 3474 | CG | TYR | A | 763 | 12.309 | 37.738 | 46.505 | 1.00 | 27.27 | C |
| ATOM | 3475 | CD1 | TYR | A | 763 | 11.959 | 37.565 | 45.165 | 1.00 | 27.29 | C |
| ATOM | 3476 | CD2 | TYR | A | 763 | 11.431 | 37.269 | 47.483 | 1.00 | 27.11 | C |
| ATOM | 3477 | CE1 | TYR | A | 763 | 10.769 | 36.944 | 44.810 | 1.00 | 27.59 | C |
| ATOM | 3478 | CE2 | TYR | A | 763 | 10.242 | 36.647 | 47.139 | 1.00 | 26.62 | C |
| ATOM | 3479 | CZ | TYR | A | 763 | 9.918 | 36.483 | 45.802 | 1.00 | 27.17 | C |
| ATOM | 3480 | OH | TYR | A | 763 | 8.745 | 35.861 | 45.446 | 1.00 | 26.84 | O |
| ATOM | 3481 | N | GLY | A | 764 | 15.211 | 40.439 | 48.696 | 1.00 | 27.17 | N |
| ATOM | 3482 | CA | GLY | A | 764 | 16.488 | 40.969 | 49.174 | 1.00 | 26.79 | C |
| ATOM | 3483 | C | GLY | A | 764 | 17.342 | 39.934 | 49.884 | 1.00 | 26.64 | C |
| ATOM | 3484 | O | GLY | A | 764 | 16.833 | 38.940 | 50.413 | 1.00 | 26.42 | O |
| ATOM | 3485 | N | ILE | A | 765 | 18.649 | 40.167 | 49.895 | 1.00 | 26.56 | N |
| ATOM | 3486 | CA | ILE | A | 765 | 19.569 | 39.246 | 50.551 | 1.00 | 26.59 | C |
| ATOM | 3487 | C | ILE | A | 765 | 20.795 | 39.936 | 51.140 | 1.00 | 26.78 | C |
| ATOM | 3488 | O | ILE | A | 765 | 21.483 | 40.694 | 50.462 | 1.00 | 26.90 | O |
| ATOM | 3489 | CB | ILE | A | 765 | 19.979 | 38.063 | 49.623 | 1.00 | 26.34 | C |
| ATOM | 3490 | CG1 | ILE | A | 765 | 20.648 | 36.949 | 50.443 | 1.00 | 26.04 | C |
| ATOM | 3491 | CG2 | ILE | A | 765 | 20.835 | 38.550 | 48.443 | 1.00 | 26.28 | C |
| ATOM | 3492 | CD1 | ILE | A | 765 | 21.025 | 35.708 | 49.648 | 1.00 | 26.28 | C |
| ATOM | 3493 | N | LEU | A | 766 | 21.032 | 39.679 | 52.422 | 1.00 | 27.11 | N |
| ATOM | 3494 | CA | LEU | A | 766 | 22.256 | 40.092 | 53.094 | 1.00 | 27.30 | C |
| ATOM | 3495 | C | LEU | A | 766 | 23.124 | 38.850 | 53.275 | 1.00 | 27.85 | C |
| ATOM | 3496 | O | LEU | A | 766 | 22.690 | 37.870 | 53.889 | 1.00 | 27.85 | O |
| ATOM | 3497 | CB | LEU | A | 766 | 21.928 | 40.739 | 54.441 | 1.00 | 27.07 | C |
| ATOM | 3498 | CG | LEU | A | 766 | 23.044 | 41.393 | 55.261 | 1.00 | 26.94 | C |
| ATOM | 3499 | CD1 | LEU | A | 766 | 23.665 | 42.592 | 54.553 | 1.00 | 25.88 | C |
| ATOM | 3500 | CD2 | LEU | A | 766 | 22.499 | 41.799 | 56.618 | 1.00 | 26.80 | C |
| ATOM | 3501 | N | TYR | A | 767 | 24.335 | 38.880 | 52.722 | 1.00 | 28.47 | N |
| ATOM | 3502 | CA | TYR | A | 767 | 25.208 | 37.706 | 52.750 | 1.00 | 28.99 | C |
| ATOM | 3503 | C | TYR | A | 767 | 26.667 | 38.001 | 53.086 | 1.00 | 29.39 | C |
| ATOM | 3504 | O | TYR | A | 767 | 27.159 | 39.112 | 52.876 | 1.00 | 29.28 | O |
| ATOM | 3505 | CB | TYR | A | 767 | 25.122 | 36.932 | 51.430 | 1.00 | 28.98 | C |
| ATOM | 3506 | CG | TYR | A | 767 | 25.578 | 37.709 | 50.216 | 1.00 | 29.26 | C |
| ATOM | 3507 | CD1 | TYR | A | 767 | 26.928 | 37.780 | 49.875 | 1.00 | 28.30 | C |
| ATOM | 3508 | CD2 | TYR | A | 767 | 24.656 | 38.366 | 49.401 | 1.00 | 29.67 | C |
| ATOM | 3509 | CE1 | TYR | A | 767 | 27.349 | 38.491 | 48.767 | 1.00 | 28.66 | C |
| ATOM | 3510 | CE2 | TYR | A | 767 | 25.069 | 39.081 | 48.282 | 1.00 | 29.59 | C |
| ATOM | 3511 | CZ | TYR | A | 767 | 26.418 | 39.135 | 47.972 | 1.00 | 29.43 | C |
| ATOM | 3512 | OH | TYR | A | 767 | 26.836 | 39.839 | 46.868 | 1.00 | 29.26 | O |
| ATOM | 3513 | N | GLU | A | 768 | 27.340 | 36.974 | 53.604 | 1.00 | 29.91 | N |
| ATOM | 3514 | CA | GLU | A | 768 | 28.771 | 36.998 | 53.868 | 1.00 | 30.18 | C |
| ATOM | 3515 | C | GLU | A | 768 | 29.555 | 36.679 | 52.600 | 1.00 | 30.96 | C |
| ATOM | 3516 | O | GLU | A | 768 | 29.289 | 35.674 | 51.926 | 1.00 | 30.73 | O |
| ATOM | 3517 | CB | GLU | A | 768 | 29.124 | 35.965 | 54.936 | 1.00 | 30.20 | C |
| ATOM | 3518 | CG | GLU | A | 768 | 28.623 | 36.302 | 56.330 | 1.00 | 29.67 | C |
| ATOM | 3519 | CD | GLU | A | 768 | 29.176 | 35.366 | 57.390 | 1.00 | 29.40 | C |
| ATOM | 3520 | OE1 | GLU | A | 768 | 29.245 | 34.141 | 57.159 | 1.00 | 27.76 | O |
| ATOM | 3521 | OE2 | GLU | A | 768 | 29.543 | 35.857 | 58.468 | 1.00 | 28.50 | O |
| ATOM | 3522 | N | HIS | A | 769 | 30.522 | 37.538 | 52.290 | 1.00 | 31.73 | N |
| ATOM | 3523 | CA | HIS | A | 769 | 31.411 | 37.363 | 51.142 | 1.00 | 32.59 | C |
| ATOM | 3524 | C | HIS | A | 769 | 32.636 | 38.254 | 51.311 | 1.00 | 32.88 | C |
| ATOM | 3525 | O | HIS | A | 769 | 32.556 | 39.317 | 51.937 | 1.00 | 32.98 | O |
| ATOM | 3526 | CB | HIS | A | 769 | 30.667 | 37.686 | 49.838 | 1.00 | 32.74 | C |
| ATOM | 3527 | CG | HIS | A | 769 | 31.554 | 37.856 | 48.642 | 1.00 | 33.80 | C |
| ATOM | 3528 | ND1 | HIS | A | 769 | 31.979 | 36.795 | 47.869 | 1.00 | 34.57 | N |
| ATOM | 3529 | CD2 | HIS | A | 769 | 32.073 | 38.968 | 48.069 | 1.00 | 34.03 | C |
| ATOM | 3530 | CE1 | HIS | A | 769 | 32.729 | 37.246 | 46.879 | 1.00 | 34.57 | C |
| ATOM | 3531 | NE2 | HIS | A | 769 | 32.801 | 38.561 | 46.978 | 1.00 | 34.49 | N |
| ATOM | 3532 | N | THR | A | 770 | 33.767 | 37.798 | 50.772 | 1.00 | 33.21 | N |
| ATOM | 3533 | CA | THR | A | 770 | 34.997 | 38.591 | 50.726 | 1.00 | 33.58 | C |
| ATOM | 3534 | C | THR | A | 770 | 35.560 | 38.679 | 49.308 | 1.00 | 33.87 | C |
| ATOM | 3535 | O | THR | A | 770 | 35.279 | 37.841 | 48.447 | 1.00 | 33.69 | O |
| ATOM | 3536 | CB | THR | A | 770 | 36.110 | 38.032 | 51.653 | 1.00 | 33.45 | C |
| ATOM | 3537 | OG1 | THR | A | 770 | 36.483 | 36.721 | 51.220 | 1.00 | 33.58 | O |
| ATOM | 3538 | CG2 | THR | A | 770 | 35.650 | 37.981 | 53.096 | 1.00 | 33.54 | C |
| ATOM | 3539 | N | GLU | A | 771 | 36.370 | 39.707 | 49.098 | 1.00 | 34.35 | N |
| ATOM | 3540 | CA | GLU | A | 771 | 37.035 | 39.968 | 47.836 | 1.00 | 35.04 | C |
| ATOM | 3541 | C | GLU | A | 771 | 38.316 | 40.703 | 48.136 | 1.00 | 34.92 | C |
| ATOM | 3542 | O | GLU | A | 771 | 38.440 | 41.336 | 49.183 | 1.00 | 35.22 | O |
| ATOM | 3543 | CB | GLU | A | 771 | 36.176 | 40.885 | 46.971 | 1.00 | 35.00 | C |
| ATOM | 3544 | CG | GLU | A | 771 | 35.432 | 40.192 | 45.847 | 1.00 | 36.22 | C |
| ATOM | 3545 | CD | GLU | A | 771 | 34.497 | 41.133 | 45.114 | 1.00 | 35.95 | C |
| ATOM | 3546 | OE1 | GLU | A | 771 | 34.646 | 42.365 | 45.264 | 1.00 | 37.47 | O |
| ATOM | 3547 | OE2 | GLU | A | 771 | 33.612 | 40.640 | 44.387 | 1.00 | 37.56 | O |
| ATOM | 3548 | N | LYS | A | 772 | 39.273 | 40.614 | 47.220 | 1.00 | 34.91 | N |
| ATOM | 3549 | CA | LYS | A | 772 | 40.395 | 41.549 | 47.190 | 1.00 | 34.87 | C |
| ATOM | 3550 | C | LYS | A | 772 | 41.043 | 41.771 | 48.571 | 1.00 | 34.34 | C |
| ATOM | 3551 | O | LYS | A | 772 | 41.366 | 42.901 | 48.947 | 1.00 | 34.65 | O |
| ATOM | 3552 | CB | LYS | A | 772 | 39.919 | 42.873 | 46.574 | 1.00 | 34.83 | C |
| ATOM | 3553 | CG | LYS | A | 772 | 39.081 | 42.678 | 45.303 | 1.00 | 35.36 | C |
| ATOM | 3554 | CD | LYS | A | 772 | 38.708 | 43.990 | 44.634 | 1.00 | 35.58 | C |
| ATOM | 3555 | CE | LYS | A | 772 | 37.933 | 43.744 | 43.337 | 1.00 | 36.38 | C |
| ATOM | 3556 | NZ | LYS | A | 772 | 37.662 | 45.024 | 42.612 | 1.00 | 36.45 | N |
| ATOM | 3557 | N | GLY | A | 773 | 41.210 | 40.684 | 49.321 | 1.00 | 33.71 | N |
| ATOM | 3558 | CA | GLY | A | 773 | 41.888 | 40.715 | 50.616 | 1.00 | 33.19 | C |
| ATOM | 3559 | C | GLY | A | 773 | 41.059 | 41.200 | 51.792 | 1.00 | 33.01 | C |
| ATOM | 3560 | O | GLY | A | 773 | 41.613 | 41.633 | 52.804 | 1.00 | 33.00 | O |
| ATOM | 3561 | N | GLN | A | 774 | 39.734 | 41.126 | 51.673 | 1.00 | 32.68 | N |
| ATOM | 3562 | CA | GLN | A | 774 | 38.853 | 41.502 | 52.779 | 1.00 | 32.45 | C |
| ATOM | 3563 | C | GLN | A | 774 | 38.888 | 40.476 | 53.915 | 1.00 | 32.12 | C |
| ATOM | 3564 | O | GLN | A | 774 | 38.921 | 39.267 | 53.674 | 1.00 | 32.15 | O |
| ATOM | 3565 | CB | GLN | A | 774 | 37.431 | 41.741 | 52.286 | 1.00 | 32.72 | C |
| ATOM | 3566 | CG | GLN | A | 774 | 37.219 | 43.124 | 51.669 | 1.00 | 32.85 | C |
| ATOM | 3567 | CD | GLN | A | 774 | 35.982 | 43.194 | 50.789 | 1.00 | 33.27 | C |
| ATOM | 3568 | OE1 | GLN | A | 774 | 35.259 | 42.211 | 50.628 | 1.00 | 33.49 | O |
| ATOM | 3569 | NE2 | GLN | A | 774 | 35.740 | 44.359 | 50.208 | 1.00 | 33.85 | N |
| ATOM | 3570 | N | ASN | A | 775 | 38.872 | 40.978 | 55.146 | 1.00 | 31.63 | N |
| ATOM | 3571 | CA | ASN | A | 775 | 39.194 | 40.189 | 56.346 | 1.00 | 31.33 | C |
| ATOM | 3572 | C | ASN | A | 775 | 38.369 | 38.927 | 56.657 | 1.00 | 31.19 | C |
| ATOM | 3573 | O | ASN | A | 775 | 38.946 | 37.908 | 57.085 | 1.00 | 31.75 | O |
| ATOM | 3574 | CB | ASN | A | 775 | 39.231 | 41.101 | 57.573 | 1.00 | 31.20 | C |
| ATOM | 3575 | CG | ASN | A | 775 | 40.473 | 41.976 | 57.615 | 1.00 | 31.07 | C |
| ATOM | 3576 | OD1 | ASN | A | 775 | 41.354 | 41.863 | 56.772 | 1.00 | 31.09 | O |
| ATOM | 3577 | ND2 | ASN | A | 775 | 40.547 | 42.849 | 58.608 | 1.00 | 31.36 | N |
| ATOM | 3578 | N | ALA | A | 776 | 37.050 | 39.000 | 56.443 | 1.00 | 30.02 | N |
| ATOM | 3579 | CA | ALA | A | 776 | 36.064 | 37.950 | 56.809 | 1.00 | 29.30 | C |
| ATOM | 3580 | C | ALA | A | 776 | 35.114 | 38.395 | 57.935 | 1.00 | 28.63 | C |
| ATOM | 3581 | O | ALA | A | 776 | 35.508 | 38.467 | 59.095 | 1.00 | 28.65 | O |
| ATOM | 3582 | CB | ALA | A | 776 | 36.732 | 36.618 | 57.163 | 1.00 | 29.10 | C |
| ATOM | 3583 | N | TYR | A | 777 | 33.880 | 38.748 | 57.597 | 1.00 | 27.88 | N |
| ATOM | 3584 | CA | TYR | A | 777 | 33.427 | 38.909 | 56.229 | 1.00 | 27.28 | C |
| ATOM | 3585 | C | TYR | A | 777 | 33.036 | 40.366 | 55.995 | 1.00 | 27.36 | C |
| ATOM | 3586 | O | TYR | A | 777 | 32.959 | 41.162 | 56.936 | 1.00 | 27.40 | O |
| ATOM | 3587 | CB | TYR | A | 777 | 32.211 | 38.020 | 55.981 | 1.00 | 26.89 | C |
| ATOM | 3588 | CG | TYR | A | 777 | 32.542 | 36.563 | 55.786 | 1.00 | 26.62 | C |
| ATOM | 3589 | CD1 | TYR | A | 777 | 32.873 | 36.073 | 54.523 | 1.00 | 26.45 | C |
| ATOM | 3590 | CD2 | TYR | A | 777 | 32.524 | 35.670 | 56.857 | 1.00 | 26.05 | C |
| ATOM | 3591 | CE1 | TYR | A | 777 | 33.181 | 34.736 | 54.329 | 1.00 | 25.38 | C |
| ATOM | 3592 | CE2 | TYR | A | 777 | 32.834 | 34.327 | 56.672 | 1.00 | 25.74 | C |
| ATOM | 3593 | CZ | TYR | A | 777 | 33.158 | 33.870 | 55.401 | 1.00 | 25.73 | C |
| ATOM | 3594 | OH | TYR | A | 777 | 33.464 | 32.547 | 55.193 | 1.00 | 26.01 | O |
| ATOM | 3595 | N | THR | A | 778 | 32.800 | 40.713 | 54.736 | 1.00 | 27.29 | N |
| ATOM | 3596 | CA | THR | A | 778 | 32.065 | 41.924 | 54.407 | 1.00 | 27.31 | C |
| ATOM | 3597 | C | THR | A | 778 | 30.631 | 41.479 | 54.121 | 1.00 | 27.40 | C |
| ATOM | 3598 | O | THR | A | 778 | 30.412 | 40.528 | 53.375 | 1.00 | 27.44 | O |
| ATOM | 3599 | CB | THR | A | 778 | 32.685 | 42.659 | 53.189 | 1.00 | 27.36 | C |
| ATOM | 3600 | OG1 | THR | A | 778 | 33.956 | 43.208 | 53.563 | 1.00 | 27.09 | O |
| ATOM | 3601 | CG2 | THR | A | 778 | 31.774 | 43.786 | 52.686 | 1.00 | 26.50 | C |
| ATOM | 3602 | N | LEU | A | 779 | 29.667 | 42.156 | 54.733 | 1.00 | 27.55 | N |
| ATOM | 3603 | CA | LEU | A | 779 | 28.253 | 41.823 | 54.558 | 1.00 | 27.65 | C |
| ATOM | 3604 | C | LEU | A | 779 | 27.594 | 42.705 | 53.502 | 1.00 | 27.69 | C |
| ATOM | 3605 | O | LEU | A | 779 | 27.549 | 43.932 | 53.640 | 1.00 | 27.70 | O |
| ATOM | 3606 | CB | LEU | A | 779 | 27.510 | 41.924 | 55.892 | 1.00 | 27.50 | C |
| ATOM | 3607 | CG | LEU | A | 779 | 27.514 | 40.707 | 56.829 | 1.00 | 27.97 | C |
| ATOM | 3608 | CD1 | LEU | A | 779 | 28.915 | 40.214 | 57.185 | 1.00 | 28.23 | C |
| ATOM | 3609 | CD2 | LEU | A | 779 | 26.733 | 41.024 | 58.097 | 1.00 | 27.73 | C |
| ATOM | 3610 | N | SER | A | 780 | 27.071 | 42.069 | 52.457 | 1.00 | 27.76 | N |
| ATOM | 3611 | CA | SER | A | 780 | 26.578 | 42.792 | 51.291 | 1.00 | 27.98 | C |
| ATOM | 3612 | C | SER | A | 780 | 25.134 | 42.475 | 50.909 | 1.00 | 28.05 | C |
| ATOM | 3613 | O | SER | A | 780 | 24.641 | 41.375 | 51.141 | 1.00 | 27.72 | O |
| ATOM | 3614 | CB | SER | A | 780 | 27.497 | 42.547 | 50.099 | 1.00 | 28.02 | C |
| ATOM | 3615 | OG | SER | A | 780 | 28.835 | 42.862 | 50.436 | 1.00 | 28.14 | O |
| ATOM | 3616 | N | PHE | A | 781 | 24.484 | 43.459 | 50.295 | 1.00 | 28.29 | N |
| ATOM | 3617 | CA | PHE | A | 781 | 23.080 | 43.374 | 49.929 | 1.00 | 28.59 | C |
| ATOM | 3618 | C | PHE | A | 781 | 22.885 | 43.289 | 48.415 | 1.00 | 28.89 | C |
| ATOM | 3619 | O | PHE | A | 781 | 23.643 | 43.885 | 47.650 | 1.00 | 29.11 | O |
| ATOM | 3620 | CB | PHE | A | 781 | 22.345 | 44.593 | 50.493 | 1.00 | 28.40 | C |
| ATOM | 3621 | CG | PHE | A | 781 | 20.908 | 44.689 | 50.086 | 1.00 | 28.19 | C |
| ATOM | 3622 | CD1 | PHE | A | 781 | 19.946 | 43.867 | 50.672 | 1.00 | 28.15 | C |
| ATOM | 3623 | CD2 | PHE | A | 781 | 20.511 | 45.612 | 49.119 | 1.00 | 27.88 | C |
| ATOM | 3624 | CE1 | PHE | A | 781 | 18.602 | 43.955 | 50.297 | 1.00 | 27.87 | C |
| ATOM | 3625 | CE2 | PHE | A | 781 | 19.178 | 45.712 | 48.736 | 1.00 | 28.61 | C |
| ATOM | 3626 | CZ | PHE | A | 781 | 18.218 | 44.878 | 49.327 | 1.00 | 28.60 | C |
| ATOM | 3627 | N | ARG | A | 782 | 21.867 | 42.540 | 48.002 | 1.00 | 29.36 | N |
| ATOM | 3628 | CA | ARG | A | 782 | 21.430 | 42.476 | 46.605 | 1.00 | 29.81 | C |
| ATOM | 3629 | C | ARG | A | 782 | 19.920 | 42.280 | 46.526 | 1.00 | 30.17 | C |
| ATOM | 3630 | O | ARG | A | 782 | 19.341 | 41.551 | 47.334 | 1.00 | 30.01 | O |
| ATOM | 3631 | CB | ARG | A | 782 | 22.095 | 41.311 | 45.875 | 1.00 | 29.58 | C |
| ATOM | 3632 | CG | ARG | A | 782 | 23.501 | 41.557 | 45.386 | 1.00 | 29.78 | C |
| ATOM | 3633 | CD | ARG | A | 782 | 23.935 | 40.365 | 44.565 | 1.00 | 30.21 | C |
| ATOM | 3634 | NE | ARG | A | 782 | 25.372 | 40.294 | 44.345 | 1.00 | 30.12 | N |
| ATOM | 3635 | CZ | ARG | A | 782 | 25.973 | 40.581 | 43.195 | 1.00 | 31.28 | C |
| ATOM | 3636 | NH1 | ARG | A | 782 | 25.264 | 40.989 | 42.148 | 1.00 | 31.63 | N |
| ATOM | 3637 | NH2 | ARG | A | 782 | 27.292 | 40.466 | 43.095 | 1.00 | 31.52 | N |
| ATOM | 3638 | N | LYS | A | 783 | 19.299 | 42.942 | 45.550 | 1.00 | 30.71 | N |
| ATOM | 3639 | CA | LYS | A | 783 | 17.909 | 42.699 | 45.181 | 1.00 | 31.48 | C |
| ATOM | 3640 | C | LYS | A | 783 | 17.902 | 41.783 | 43.975 | 1.00 | 31.67 | C |
| ATOM | 3641 | O | LYS | A | 783 | 18.860 | 41.772 | 43.196 | 1.00 | 31.82 | O |
| ATOM | 3642 | CB | LYS | A | 783 | 17.203 | 43.996 | 44.767 | 1.00 | 31.69 | C |
| ATOM | 3643 | CG | LYS | A | 783 | 16.767 | 44.921 | 45.881 | 1.00 | 32.91 | C |
| ATOM | 3644 | CD | LYS | A | 783 | 16.670 | 46.357 | 45.350 | 1.00 | 35.08 | C |
| ATOM | 3645 | CE | LYS | A | 783 | 15.713 | 47.208 | 46.176 | 1.00 | 35.63 | C |
| ATOM | 3646 | NZ | LYS | A | 783 | 15.889 | 48.671 | 45.926 | 1.00 | 35.71 | N |
| ATOM | 3647 | N | PHE | A | 784 | 16.814 | 41.035 | 43.813 | 1.00 | 31.77 | N |
| ATOM | 3648 | CA | PHE | A | 784 | 16.551 | 40.305 | 42.574 | 1.00 | 31.93 | C |
| ATOM | 3649 | C | PHE | A | 784 | 15.052 | 40.128 | 42.354 | 1.00 | 32.26 | C |
| ATOM | 3650 | O | PHE | A | 784 | 14.257 | 40.329 | 43.278 | 1.00 | 32.23 | O |
| ATOM | 3651 | CB | PHE | A | 784 | 17.286 | 38.956 | 42.535 | 1.00 | 31.64 | C |
| ATOM | 3652 | CG | PHE | A | 784 | 17.066 | 38.096 | 43.746 | 1.00 | 31.27 | C |
| ATOM | 3653 | CD1 | PHE | A | 784 | 16.009 | 37.187 | 43.790 | 1.00 | 30.95 | C |
| ATOM | 3654 | CD2 | PHE | A | 784 | 17.927 | 38.178 | 44.837 | 1.00 | 30.62 | C |
| ATOM | 3655 | CE1 | PHE | A | 784 | 15.808 | 36.380 | 44.905 | 1.00 | 30.82 | C |
| ATOM | 3656 | CE2 | PHE | A | 784 | 17.737 | 37.374 | 45.958 | 1.00 | 30.61 | C |
| ATOM | 3657 | CZ | PHE | A | 784 | 16.676 | 36.474 | 45.995 | 1.00 | 30.77 | C |
| ATOM | 3658 | N | ASN | A | 785 | 14.679 | 39.764 | 41.128 | 1.00 | 32.73 | N |
| ATOM | 3659 | CA | ASN | A | 785 | 13.281 | 39.519 | 40.769 | 1.00 | 33.29 | C |
| ATOM | 3660 | C | ASN | A | 785 | 12.985 | 38.038 | 40.508 | 1.00 | 33.79 | C |
| ATOM | 3661 | O | ASN | A | 785 | 13.860 | 37.184 | 40.674 | 1.00 | 33.86 | O |
| ATOM | 3662 | CB | ASN | A | 785 | 12.870 | 40.378 | 39.567 | 1.00 | 33.13 | C |
| ATOM | 3663 | CG | ASN | A | 785 | 13.790 | 40.196 | 38.366 | 1.00 | 32.99 | C |
| ATOM | 3664 | OD1 | ASN | A | 785 | 14.340 | 39.114 | 38.132 | 1.00 | 32.29 | O |
| ATOM | 3665 | ND2 | ASN | A | 785 | 13.960 | 41.266 | 37.597 | 1.00 | 32.58 | N |
| ATOM | 3666 | N | TRP | A | 786 | 11.754 | 37.749 | 40.091 | 1.00 | 34.51 | N |
| ATOM | 3667 | CA | TRP | A | 786 | 11.307 | 36.380 | 39.863 | 1.00 | 35.28 | C |
| ATOM | 3668 | C | TRP | A | 786 | 11.993 | 35.725 | 38.663 | 1.00 | 35.83 | C |
| ATOM | 3669 | O | TRP | A | 786 | 12.202 | 34.509 | 38.662 | 1.00 | 36.17 | O |
| ATOM | 3670 | CB | TRP | A | 786 | 9.785 | 36.326 | 39.703 | 1.00 | 35.53 | C |
| ATOM | 3671 | CG | TRP | A | 786 | 9.267 | 34.923 | 39.617 | 1.00 | 35.98 | C |
| ATOM | 3672 | CD1 | TRP | A | 786 | 8.942 | 34.236 | 38.480 | 1.00 | 36.17 | C |
| ATOM | 3673 | CD2 | TRP | A | 786 | 9.054 | 34.020 | 40.707 | 1.00 | 35.84 | C |
| ATOM | 3674 | NE1 | TRP | A | 786 | 8.524 | 32.967 | 38.797 | 1.00 | 36.52 | N |
| ATOM | 3675 | CE2 | TRP | A | 786 | 8.585 | 32.805 | 40.157 | 1.00 | 36.11 | C |
| ATOM | 3676 | CE3 | TRP | A | 786 | 9.207 | 34.121 | 42.096 | 1.00 | 35.63 | C |
| ATOM | 3677 | CZ2 | TRP | A | 786 | 8.265 | 31.697 | 40.948 | 1.00 | 35.77 | C |
| ATOM | 3678 | CZ3 | TRP | A | 786 | 8.885 | 33.019 | 42.884 | 1.00 | 35.89 | C |
| ATOM | 3679 | CH2 | TRP | A | 786 | 8.423 | 31.823 | 42.305 | 1.00 | 35.85 | C |
| ATOM | 3680 | N | ASP | A | 787 | 12.329 | 36.529 | 37.652 | 1.00 | 36.19 | N |
| ATOM | 3681 | CA | ASP | A | 787 | 13.050 | 36.055 | 36.463 | 1.00 | 36.61 | C |
| ATOM | 3682 | C | ASP | A | 787 | 14.412 | 35.480 | 36.829 | 1.00 | 36.96 | C |
| ATOM | 3683 | O | ASP | A | 787 | 14.829 | 34.459 | 36.273 | 1.00 | 37.08 | O |
| ATOM | 3684 | CB | ASP | A | 787 | 13.233 | 37.183 | 35.434 | 1.00 | 36.51 | C |
| ATOM | 3685 | CG | ASP | A | 787 | 11.992 | 37.417 | 34.576 | 1.00 | 36.46 | C |
| ATOM | 3686 | OD1 | ASP | A | 787 | 11.039 | 36.603 | 34.640 | 1.00 | 36.27 | O |
| ATOM | 3687 | OD2 | ASP | A | 787 | 11.977 | 38.421 | 33.830 | 1.00 | 35.51 | O |
| ATOM | 3688 | N | PHE | A | 788 | 15.097 | 36.140 | 37.763 | 1.00 | 37.24 | N |
| ATOM | 3689 | CA | PHE | A | 788 | 16.381 | 35.660 | 38.257 | 1.00 | 37.61 | C |
| ATOM | 3690 | C | PHE | A | 788 | 16.238 | 34.291 | 38.914 | 1.00 | 37.92 | C |
| ATOM | 3691 | O | PHE | A | 788 | 17.173 | 33.490 | 38.912 | 1.00 | 37.81 | O |
| ATOM | 3692 | CB | PHE | A | 788 | 16.998 | 36.652 | 39.246 | 1.00 | 37.56 | C |
| ATOM | 3693 | CG | PHE | A | 788 | 18.275 | 36.156 | 39.873 | 1.00 | 37.90 | C |
| ATOM | 3694 | CD1 | PHE | A | 788 | 19.475 | 36.193 | 39.164 | 1.00 | 37.33 | C |
| ATOM | 3695 | CD2 | PHE | A | 788 | 18.275 | 35.632 | 41.164 | 1.00 | 38.18 | C |
| ATOM | 3696 | CE1 | PHE | A | 788 | 20.652 | 35.723 | 39.729 | 1.00 | 37.41 | C |
| ATOM | 3697 | CE2 | PHE | A | 788 | 19.453 | 35.159 | 41.740 | 1.00 | 38.38 | C |
| ATOM | 3698 | CZ | PHE | A | 788 | 20.645 | 35.208 | 41.019 | 1.00 | 37.63 | C |
| ATOM | 3699 | N | LEU | A | 789 | 15.057 | 34.033 | 39.468 | 1.00 | 38.47 | N |
| ATOM | 3700 | CA | LEU | A | 789 | 14.788 | 32.797 | 40.191 | 1.00 | 38.74 | C |
| ATOM | 3701 | C | LEU | A | 789 | 14.315 | 31.661 | 39.301 | 1.00 | 39.06 | C |
| ATOM | 3702 | O | LEU | A | 789 | 14.682 | 30.507 | 39.523 | 1.00 | 38.92 | O |
| ATOM | 3703 | CB | LEU | A | 789 | 13.744 | 33.051 | 41.272 | 1.00 | 38.77 | C |
| ATOM | 3704 | CG | LEU | A | 789 | 14.252 | 33.631 | 42.587 | 1.00 | 38.85 | C |
| ATOM | 3705 | CD1 | LEU | A | 789 | 13.138 | 34.385 | 43.290 | 1.00 | 38.34 | C |
| ATOM | 3706 | CD2 | LEU | A | 789 | 14.804 | 32.512 | 43.459 | 1.00 | 38.53 | C |
| ATOM | 3707 | N | SER | A | 790 | 13.504 | 31.992 | 38.298 | 1.00 | 39.53 | N |
| ATOM | 3708 | CA | SER | A | 790 | 12.776 | 30.983 | 37.528 | 1.00 | 40.12 | C |
| ATOM | 3709 | C | SER | A | 790 | 13.377 | 30.638 | 36.156 | 1.00 | 40.38 | C |
| ATOM | 3710 | O | SER | A | 790 | 13.124 | 29.555 | 35.621 | 1.00 | 40.61 | O |
| ATOM | 3711 | CB | SER | A | 790 | 11.309 | 31.393 | 37.379 | 1.00 | 40.10 | C |
| ATOM | 3712 | OG | SER | A | 790 | 11.188 | 32.567 | 36.596 | 1.00 | 40.62 | O |
| ATOM | 3713 | N | LYS | A | 791 | 14.161 | 31.555 | 35.593 | 1.00 | 40.67 | N |
| ATOM | 3714 | CA | LYS | A | 791 | 14.769 | 31.350 | 34.276 | 1.00 | 40.92 | C |
| ATOM | 3715 | C | LYS | A | 791 | 16.163 | 31.963 | 34.171 | 1.00 | 40.88 | C |
| ATOM | 3716 | O | LYS | A | 791 | 17.053 | 31.386 | 33.546 | 1.00 | 40.94 | O |
| ATOM | 3717 | CB | LYS | A | 791 | 13.861 | 31.897 | 33.166 | 1.00 | 41.08 | C |
| ATOM | 3718 | CG | LYS | A | 791 | 13.522 | 33.386 | 33.293 | 1.00 | 41.13 | C |
| ATOM | 3719 | CD | LYS | A | 791 | 12.691 | 33.883 | 32.121 | 1.00 | 41.00 | C |
| ATOM | 3720 | CE | LYS | A | 791 | 11.264 | 33.354 | 32.177 | 1.00 | 41.36 | C |
| ATOM | 3721 | NZ | LYS | A | 791 | 10.317 | 34.245 | 31.442 | 1.00 | 41.47 | N |
| TER | 3722 | | LYS | A | 791 | | | | | | |
| ATOM | 3723 | N | ALA | B | 322 | 26.470 | −5.993 | 127.959 | 1.00 | 69.14 | N |
| ATOM | 3724 | CA | ALA | B | 322 | 25.819 | −7.228 | 127.433 | 1.00 | 69.17 | C |
| ATOM | 3725 | C | ALA | B | 322 | 25.536 | −7.095 | 125.941 | 1.00 | 69.25 | C |
| ATOM | 3726 | O | ALA | B | 322 | 25.639 | −6.002 | 125.378 | 1.00 | 69.21 | O |
| ATOM | 3727 | CB | ALA | B | 322 | 24.530 | −7.523 | 128.201 | 1.00 | 69.07 | C |
| ATOM | 3728 | N | LEU | B | 323 | 25.197 | −8.220 | 125.311 | 1.00 | 69.27 | N |
| ATOM | 3729 | CA | LEU | B | 323 | 24.792 | −8.265 | 123.907 | 1.00 | 69.18 | C |
| ATOM | 3730 | C | LEU | B | 323 | 23.977 | −9.529 | 123.652 | 1.00 | 69.12 | C |
| ATOM | 3731 | O | LEU | B | 323 | 24.427 | −10.638 | 123.960 | 1.00 | 69.24 | O |
| ATOM | 3732 | CB | LEU | B | 323 | 26.016 | −8.213 | 122.980 | 1.00 | 69.26 | C |
| ATOM | 3733 | CG | LEU | B | 323 | 25.855 | −8.541 | 121.484 | 1.00 | 69.38 | C |
| ATOM | 3734 | CD1 | LEU | B | 323 | 24.853 | −7.622 | 120.792 | 1.00 | 69.30 | C |
| ATOM | 3735 | CD2 | LEU | B | 323 | 27.202 | −8.491 | 120.773 | 1.00 | 69.28 | C |
| ATOM | 3736 | N | THR | B | 324 | 22.783 | −9.355 | 123.091 | 1.00 | 68.91 | N |
| ATOM | 3737 | CA | THR | B | 324 | 21.884 | −10.479 | 122.819 | 1.00 | 68.76 | C |
| ATOM | 3738 | C | THR | B | 324 | 22.362 | −11.300 | 121.621 | 1.00 | 68.62 | C |
| ATOM | 3739 | O | THR | B | 324 | 23.011 | −10.772 | 120.716 | 1.00 | 68.63 | O |
| ATOM | 3740 | CB | THR | B | 324 | 20.429 | −10.013 | 122.577 | 1.00 | 68.73 | C |
| ATOM | 3741 | OG1 | THR | B | 324 | 20.400 | −9.064 | 121.505 | 1.00 | 68.97 | O |
| ATOM | 3742 | CG2 | THR | B | 324 | 19.848 | −9.374 | 123.832 | 1.00 | 68.60 | C |
| ATOM | 3743 | N | GLU | B | 325 | 22.038 | −12.592 | 121.631 | 1.00 | 68.42 | N |
| ATOM | 3744 | CA | GLU | B | 325 | 22.423 | −13.503 | 120.554 | 1.00 | 68.30 | C |
| ATOM | 3745 | C | GLU | B | 325 | 21.633 | −13.257 | 119.267 | 1.00 | 67.98 | C |
| ATOM | 3746 | O | GLU | B | 325 | 20.596 | −12.587 | 119.275 | 1.00 | 67.93 | O |
| ATOM | 3747 | CB | GLU | B | 325 | 22.297 | −14.966 | 121.001 | 1.00 | 68.28 | C |
| ATOM | 3748 | CG | GLU | B | 325 | 23.494 | −15.471 | 121.813 | 1.00 | 68.73 | C |
| ATOM | 3749 | CD | GLU | B | 325 | 23.454 | −16.974 | 122.089 | 1.00 | 68.74 | C |
| ATOM | 3750 | OE1 | GLU | B | 325 | 22.463 | −17.459 | 122.682 | 1.00 | 69.26 | O |
| ATOM | 3751 | OE2 | GLU | B | 325 | 24.428 | −17.669 | 121.726 | 1.00 | 69.19 | O |
| ATOM | 3752 | N | LYS | B | 326 | 22.148 | −13.809 | 118.170 | 1.00 | 67.58 | N |
| ATOM | 3753 | CA | LYS | B | 326 | 21.582 | −13.659 | 116.830 | 1.00 | 67.11 | C |
| ATOM | 3754 | C | LYS | B | 326 | 20.105 | −14.065 | 116.766 | 1.00 | 66.67 | C |
| ATOM | 3755 | O | LYS | B | 326 | 19.738 | −15.181 | 117.139 | 1.00 | 66.63 | O |
| ATOM | 3756 | CB | LYS | B | 326 | 22.402 | −14.500 | 115.845 | 1.00 | 67.22 | C |
| ATOM | 3757 | CG | LYS | B | 326 | 22.231 | −14.144 | 114.382 | 1.00 | 67.41 | C |
| ATOM | 3758 | CD | LYS | B | 326 | 22.678 | −15.300 | 113.498 | 1.00 | 67.80 | C |
| ATOM | 3759 | CE | LYS | B | 326 | 22.830 | −14.869 | 112.046 | 1.00 | 68.19 | C |
| ATOM | 3760 | NZ | LYS | B | 326 | 22.902 | −16.032 | 111.113 | 1.00 | 68.21 | N |
| ATOM | 3761 | N | THR | B | 327 | 19.270 | −13.143 | 116.294 | 1.00 | 66.14 | N |
| ATOM | 3762 | CA | THR | B | 327 | 17.838 | −13.389 | 116.125 | 1.00 | 65.57 | C |
| ATOM | 3763 | C | THR | B | 327 | 17.415 | −13.021 | 114.703 | 1.00 | 65.20 | C |
| ATOM | 3764 | O | THR | B | 327 | 17.288 | −11.841 | 114.367 | 1.00 | 65.17 | O |
| ATOM | 3765 | CB | THR | B | 327 | 16.997 | −12.590 | 117.150 | 1.00 | 65.60 | C |
| ATOM | 3766 | OG1 | THR | B | 327 | 17.517 | −12.802 | 118.466 | 1.00 | 65.49 | O |
| ATOM | 3767 | CG2 | THR | B | 327 | 15.532 | −13.019 | 117.110 | 1.00 | 65.49 | C |
| ATOM | 3768 | N | ASP | B | 328 | 17.200 | −14.043 | 113.878 | 1.00 | 64.62 | N |
| ATOM | 3769 | CA | ASP | B | 328 | 16.871 | −13.860 | 112.466 | 1.00 | 64.01 | C |
| ATOM | 3770 | C | ASP | B | 328 | 15.414 | −13.443 | 112.265 | 1.00 | 63.82 | C |
| ATOM | 3771 | O | ASP | B | 328 | 14.504 | −14.048 | 112.838 | 1.00 | 63.73 | O |
| ATOM | 3772 | CB | ASP | B | 328 | 17.165 | −15.145 | 111.681 | 1.00 | 63.92 | C |
| ATOM | 3773 | CG | ASP | B | 328 | 18.545 | −15.727 | 111.985 | 1.00 | 63.38 | C |
| ATOM | 3774 | OD1 | ASP | B | 328 | 19.417 | −15.003 | 112.514 | 1.00 | 62.54 | O |
| ATOM | 3775 | OD2 | ASP | B | 328 | 18.753 | −16.922 | 111.689 | 1.00 | 62.45 | O |
| ATOM | 3776 | N | ILE | B | 329 | 15.204 | −12.404 | 111.455 | 1.00 | 63.51 | N |
| ATOM | 3777 | CA | ILE | B | 329 | 13.856 | −11.933 | 111.112 | 1.00 | 63.20 | C |
| ATOM | 3778 | C | ILE | B | 329 | 13.478 | −12.323 | 109.678 | 1.00 | 62.98 | C |
| ATOM | 3779 | O | ILE | B | 329 | 12.386 | −12.845 | 109.437 | 1.00 | 62.95 | O |
| ATOM | 3780 | CB | ILE | B | 329 | 13.683 | −10.393 | 111.322 | 1.00 | 63.19 | C |
| ATOM | 3781 | CG1 | ILE | B | 329 | 13.882 | −10.005 | 112.789 | 1.00 | 63.18 | C |
| ATOM | 3782 | CG2 | ILE | B | 329 | 12.300 | −9.924 | 110.878 | 1.00 | 63.09 | C |
| ATOM | 3783 | CD1 | ILE | B | 329 | 15.287 | −9.605 | 113.132 | 1.00 | 62.92 | C |
| ATOM | 3784 | N | PHE | B | 330 | 14.384 | −12.069 | 108.737 | 1.00 | 62.72 | N |
| ATOM | 3785 | CA | PHE | B | 330 | 14.147 | −12.381 | 107.329 | 1.00 | 62.53 | C |
| ATOM | 3786 | C | PHE | B | 330 | 15.121 | −13.451 | 106.841 | 1.00 | 62.57 | C |
| ATOM | 3787 | O | PHE | B | 330 | 16.210 | −13.141 | 106.358 | 1.00 | 62.52 | O |
| ATOM | 3788 | CB | PHE | B | 330 | 14.254 | −11.119 | 106.467 | 1.00 | 62.36 | C |
| ATOM | 3789 | CG | PHE | B | 330 | 13.293 | −10.027 | 106.853 | 1.00 | 61.98 | C |
| ATOM | 3790 | CD1 | PHE | B | 330 | 11.946 | −10.110 | 106.514 | 1.00 | 61.73 | C |
| ATOM | 3791 | CD2 | PHE | B | 330 | 13.740 | −8.900 | 107.541 | 1.00 | 61.74 | C |
| ATOM | 3792 | CE1 | PHE | B | 330 | 11.056 | −9.092 | 106.864 | 1.00 | 61.58 | C |
| ATOM | 3793 | CE2 | PHE | B | 330 | 12.859 | −7.877 | 107.892 | 1.00 | 61.45 | C |
| ATOM | 3794 | CZ | PHE | B | 330 | 11.515 | −7.975 | 107.554 | 1.00 | 61.49 | C |
| ATOM | 3795 | N | GLU | B | 331 | 14.707 | −14.709 | 106.965 | 1.00 | 62.71 | N |
| ATOM | 3796 | CA | GLU | B | 331 | 15.574 | −15.859 | 106.707 | 1.00 | 62.84 | C |
| ATOM | 3797 | C | GLU | B | 331 | 15.738 | −16.136 | 105.216 | 1.00 | 62.73 | C |
| ATOM | 3798 | O | GLU | B | 331 | 14.754 | −16.335 | 104.506 | 1.00 | 62.72 | O |
| ATOM | 3799 | CB | GLU | B | 331 | 15.018 | −17.106 | 107.405 | 1.00 | 62.98 | C |
| ATOM | 3800 | CG | GLU | B | 331 | 14.803 | −16.954 | 108.913 | 1.00 | 63.55 | C |
| ATOM | 3801 | CD | GLU | B | 331 | 13.620 | −17.765 | 109.436 | 1.00 | 64.17 | C |
| ATOM | 3802 | OE1 | GLU | B | 331 | 12.761 | −18.181 | 108.624 | 1.00 | 64.29 | O |
| ATOM | 3803 | OE2 | GLU | B | 331 | 13.544 | −17.974 | 110.668 | 1.00 | 63.91 | O |
| ATOM | 3804 | N | SER | B | 332 | 16.986 | −16.150 | 104.752 | 1.00 | 62.76 | N |
| ATOM | 3805 | CA | SER | B | 332 | 17.296 | −16.515 | 103.368 | 1.00 | 62.82 | C |
| ATOM | 3806 | C | SER | B | 332 | 17.361 | −18.038 | 103.197 | 1.00 | 62.98 | C |
| ATOM | 3807 | O | SER | B | 332 | 17.080 | −18.787 | 104.133 | 1.00 | 62.96 | O |
| ATOM | 3808 | CB | SER | B | 332 | 18.597 | −15.846 | 102.898 | 1.00 | 62.80 | C |
| ATOM | 3809 | OG | SER | B | 332 | 19.651 | −16.019 | 103.830 | 1.00 | 62.55 | O |
| ATOM | 3810 | N | GLY | B | 333 | 17.715 | −18.487 | 101.995 | 1.00 | 63.14 | N |
| ATOM | 3811 | CA | GLY | B | 333 | 17.858 | −19.915 | 101.712 | 1.00 | 63.27 | C |
| ATOM | 3812 | C | GLY | B | 333 | 19.275 | −20.419 | 101.920 | 1.00 | 63.40 | C |
| ATOM | 3813 | O | GLY | B | 333 | 20.146 | −19.683 | 102.395 | 1.00 | 63.31 | O |
| ATOM | 3814 | N | ARG | B | 334 | 19.504 | −21.678 | 101.555 | 1.00 | 63.54 | N |
| ATOM | 3815 | CA | ARG | B | 334 | 20.798 | −22.324 | 101.758 | 1.00 | 63.80 | C |
| ATOM | 3816 | C | ARG | B | 334 | 21.320 | −22.954 | 100.470 | 1.00 | 63.91 | C |
| ATOM | 3817 | O | ARG | B | 334 | 20.581 | −23.652 | 99.774 | 1.00 | 63.99 | O |
| ATOM | 3818 | CB | ARG | B | 334 | 20.698 | −23.408 | 102.838 | 1.00 | 63.90 | C |
| ATOM | 3819 | CG | ARG | B | 334 | 20.417 | −22.916 | 104.250 | 1.00 | 64.03 | C |
| ATOM | 3820 | CD | ARG | B | 334 | 20.156 | −24.102 | 105.161 | 1.00 | 64.39 | C |
| ATOM | 3821 | NE | ARG | B | 334 | 19.840 | −23.702 | 106.530 | 1.00 | 64.70 | N |
| ATOM | 3822 | CZ | ARG | B | 334 | 19.429 | −24.539 | 107.479 | 1.00 | 64.85 | C |
| ATOM | 3823 | NH1 | ARG | B | 334 | 19.276 | −25.832 | 107.213 | 1.00 | 64.82 | N |
| ATOM | 3824 | NH2 | ARG | B | 334 | 19.168 | −24.083 | 108.698 | 1.00 | 64.73 | N |
| ATOM | 3825 | N | ASN | B | 335 | 22.593 | −22.700 | 100.166 | 1.00 | 64.00 | N |
| ATOM | 3826 | CA | ASN | B | 335 | 23.313 | −23.353 | 99.059 | 1.00 | 64.12 | C |
| ATOM | 3827 | C | ASN | B | 335 | 22.664 | −23.215 | 97.673 | 1.00 | 64.08 | C |
| ATOM | 3828 | O | ASN | B | 335 | 22.868 | −24.061 | 96.795 | 1.00 | 63.98 | O |
| ATOM | 3829 | CB | ASN | B | 335 | 23.579 | −24.832 | 99.391 | 1.00 | 64.22 | C |
| ATOM | 3830 | CG | ASN | B | 335 | 24.155 | −25.025 | 100.785 | 1.00 | 64.56 | C |
| ATOM | 3831 | OD1 | ASN | B | 335 | 23.458 | −25.469 | 101.699 | 1.00 | 64.80 | O |
| ATOM | 3832 | ND2 | ASN | B | 335 | 25.428 | −24.676 | 100.958 | 1.00 | 64.83 | N |
| ATOM | 3833 | N | GLY | B | 336 | 21.895 | −22.144 | 97.483 | 1.00 | 64.09 | N |
| ATOM | 3834 | CA | GLY | B | 336 | 21.187 | −21.905 | 96.225 | 1.00 | 64.03 | C |
| ATOM | 3835 | C | GLY | B | 336 | 19.765 | −22.441 | 96.228 | 1.00 | 64.02 | C |
| ATOM | 3836 | O | GLY | B | 336 | 19.088 | −22.426 | 95.195 | 1.00 | 64.08 | O |
| ATOM | 3837 | N | LYS | B | 337 | 19.318 | −22.923 | 97.388 | 1.00 | 63.84 | N |
| ATOM | 3838 | CA | LYS | B | 337 | 17.941 | −23.383 | 97.567 | 1.00 | 63.62 | C |
| ATOM | 3839 | C | LYS | B | 337 | 17.048 | −22.224 | 98.003 | 1.00 | 63.32 | C |
| ATOM | 3840 | O | LYS | B | 337 | 17.490 | −21.348 | 98.751 | 1.00 | 63.29 | O |
| ATOM | 3841 | CB | LYS | B | 337 | 17.859 | −24.497 | 98.619 | 1.00 | 63.67 | C |
| ATOM | 3842 | CG | LYS | B | 337 | 18.505 | −25.822 | 98.227 | 1.00 | 63.83 | C |
| ATOM | 3843 | CD | LYS | B | 337 | 18.422 | −26.824 | 99.376 | 1.00 | 63.70 | C |
| ATOM | 3844 | CE | LYS | B | 337 | 19.151 | −28.121 | 99.051 | 1.00 | 64.22 | C |
| ATOM | 3845 | NZ | LYS | B | 337 | 18.449 | −28.928 | 98.013 | 1.00 | 64.12 | N |
| ATOM | 3846 | N | PRO | B | 338 | 15.788 | −22.211 | 97.532 | 1.00 | 62.95 | N |
| ATOM | 3847 | CA | PRO | B | 338 | 14.811 | −21.282 | 98.090 | 1.00 | 62.64 | C |
| ATOM | 3848 | C | PRO | B | 338 | 14.388 | −21.756 | 99.481 | 1.00 | 62.30 | C |
| ATOM | 3849 | O | PRO | B | 338 | 14.331 | −22.963 | 99.723 | 1.00 | 62.36 | O |
| ATOM | 3850 | CB | PRO | B | 338 | 13.629 | −21.380 | 97.115 | 1.00 | 62.69 | C |
| ATOM | 3851 | CG | PRO | B | 338 | 14.117 | −22.183 | 95.946 | 1.00 | 62.85 | C |
| ATOM | 3852 | CD | PRO | B | 338 | 15.207 | −23.043 | 96.465 | 1.00 | 62.91 | C |
| ATOM | 3853 | N | ASN | B | 339 | 14.099 | −20.821 | 100.385 | 1.00 | 61.79 | N |
| ATOM | 3854 | CA | ASN | B | 339 | 13.695 | −21.172 | 101.750 | 1.00 | 61.26 | C |
| ATOM | 3855 | C | ASN | B | 339 | 12.277 | −21.760 | 101.808 | 1.00 | 61.01 | C |
| ATOM | 3856 | O | ASN | B | 339 | 11.712 | −22.120 | 100.770 | 1.00 | 61.00 | O |
| ATOM | 3857 | CB | ASN | B | 339 | 13.857 | −19.970 | 102.696 | 1.00 | 61.23 | C |
| ATOM | 3858 | CG | ASN | B | 339 | 12.827 | −18.865 | 102.451 | 1.00 | 61.10 | C |
| ATOM | 3859 | OD1 | ASN | B | 339 | 12.101 | −18.867 | 101.455 | 1.00 | 60.91 | O |
| ATOM | 3860 | ND2 | ASN | B | 339 | 12.769 | −17.910 | 103.371 | 1.00 | 60.82 | N |
| ATOM | 3861 | N | LYS | B | 340 | 11.713 | −21.855 | 103.015 | 1.00 | 60.55 | N |
| ATOM | 3862 | CA | LYS | B | 340 | 10.356 | −22.384 | 103.225 | 1.00 | 60.08 | C |
| ATOM | 3863 | C | LYS | B | 340 | 9.328 | −21.820 | 102.242 | 1.00 | 59.62 | C |
| ATOM | 3864 | O | LYS | B | 340 | 8.492 | −22.556 | 101.717 | 1.00 | 59.58 | O |
| ATOM | 3865 | CB | LYS | B | 340 | 9.881 | −22.112 | 104.657 | 1.00 | 60.17 | C |
| ATOM | 3866 | CG | LYS | B | 340 | 10.681 | −22.808 | 105.739 | 1.00 | 60.43 | C |
| ATOM | 3867 | CD | LYS | B | 340 | 10.045 | −22.605 | 107.104 | 1.00 | 60.72 | C |
| ATOM | 3868 | CE | LYS | B | 340 | 10.822 | −23.351 | 108.176 | 1.00 | 61.28 | C |
| ATOM | 3869 | NZ | LYS | B | 340 | 10.024 | −23.535 | 109.418 | 1.00 | 61.76 | N |
| ATOM | 3870 | N | ASP | B | 341 | 9.405 | −20.513 | 101.998 | 1.00 | 59.05 | N |
| ATOM | 3871 | CA | ASP | B | 341 | 8.408 | −19.806 | 101.196 | 1.00 | 58.44 | C |
| ATOM | 3872 | C | ASP | B | 341 | 8.886 | −19.510 | 99.772 | 1.00 | 57.87 | C |
| ATOM | 3873 | O | ASP | B | 341 | 8.345 | −18.627 | 99.099 | 1.00 | 57.85 | O |
| ATOM | 3874 | CB | ASP | B | 341 | 7.989 | −18.518 | 101.908 | 1.00 | 58.48 | C |
| ATOM | 3875 | CG | ASP | B | 341 | 7.479 | −18.770 | 103.312 | 1.00 | 58.61 | C |
| ATOM | 3876 | OD1 | ASP | B | 341 | 6.377 | −19.340 | 103.457 | 1.00 | 59.00 | O |
| ATOM | 3877 | OD2 | ASP | B | 341 | 8.180 | −18.396 | 104.274 | 1.00 | 58.74 | O |
| ATOM | 3878 | N | GLY | B | 342 | 9.897 | −20.251 | 99.323 | 1.00 | 57.12 | N |
| ATOM | 3879 | CA | GLY | B | 342 | 10.416 | −20.121 | 97.962 | 1.00 | 56.17 | C |
| ATOM | 3880 | C | GLY | B | 342 | 11.148 | −18.818 | 97.689 | 1.00 | 55.49 | C |
| ATOM | 3881 | O | GLY | B | 342 | 11.327 | −18.432 | 96.531 | 1.00 | 55.55 | O |
| ATOM | 3882 | N | ILE | B | 343 | 11.567 | −18.141 | 98.758 | 1.00 | 54.67 | N |
| ATOM | 3883 | CA | ILE | B | 343 | 12.300 | −16.882 | 98.651 | 1.00 | 53.81 | C |
| ATOM | 3884 | C | ILE | B | 343 | 13.782 | −17.135 | 98.910 | 1.00 | 53.22 | C |
| ATOM | 3885 | O | ILE | B | 343 | 14.188 | −17.402 | 100.043 | 1.00 | 53.13 | O |
| ATOM | 3886 | CB | ILE | B | 343 | 11.741 | −15.802 | 99.620 | 1.00 | 53.81 | C |
| ATOM | 3887 | CG1 | ILE | B | 343 | 10.280 | −15.486 | 99.277 | 1.00 | 53.56 | C |
| ATOM | 3888 | CG2 | ILE | B | 343 | 12.590 | −14.527 | 99.560 | 1.00 | 53.74 | C |
| ATOM | 3889 | CD1 | ILE | B | 343 | 9.454 | −14.982 | 100.438 | 1.00 | 53.10 | C |
| ATOM | 3890 | N | LYS | B | 344 | 14.581 | −17.058 | 97.848 | 1.00 | 52.41 | N |
| ATOM | 3891 | CA | LYS | B | 344 | 16.008 | −17.344 | 97.939 | 1.00 | 51.68 | C |
| ATOM | 3892 | C | LYS | B | 344 | 16.726 | −16.341 | 98.832 | 1.00 | 51.21 | C |
| ATOM | 3893 | O | LYS | B | 344 | 17.524 | −16.727 | 99.685 | 1.00 | 51.21 | O |
| ATOM | 3894 | CB | LYS | B | 344 | 16.671 | −17.356 | 96.554 | 1.00 | 51.70 | C |
| ATOM | 3895 | CG | LYS | B | 344 | 16.393 | −18.578 | 95.688 | 1.00 | 51.34 | C |
| ATOM | 3896 | CD | LYS | B | 344 | 17.526 | −18.774 | 94.677 | 1.00 | 51.30 | C |
| ATOM | 3897 | CE | LYS | B | 344 | 17.070 | −19.489 | 93.404 | 1.00 | 50.83 | C |
| ATOM | 3898 | NZ | LYS | B | 344 | 16.747 | −20.928 | 93.594 | 1.00 | 50.19 | N |
| ATOM | 3899 | N | SER | B | 345 | 16.426 | −15.059 | 98.645 | 1.00 | 50.60 | N |
| ATOM | 3900 | CA | SER | B | 345 | 17.246 | −14.003 | 99.222 | 1.00 | 49.95 | C |
| ATOM | 3901 | C | SER | B | 345 | 16.479 | −12.922 | 99.966 | 1.00 | 49.55 | C |
| ATOM | 3902 | O | SER | B | 345 | 15.364 | −12.559 | 99.592 | 1.00 | 49.48 | O |
| ATOM | 3903 | CB | SER | B | 345 | 18.099 | −13.356 | 98.131 | 1.00 | 49.98 | C |
| ATOM | 3904 | OG | SER | B | 345 | 18.742 | −12.191 | 98.615 | 1.00 | 49.84 | O |
| ATOM | 3905 | N | TYR | B | 346 | 17.106 | −12.421 | 101.026 | 1.00 | 49.13 | N |
| ATOM | 3906 | CA | TYR | B | 346 | 16.664 | −11.217 | 101.720 | 1.00 | 48.73 | C |
| ATOM | 3907 | C | TYR | B | 346 | 17.832 | −10.251 | 101.803 | 1.00 | 48.46 | C |
| ATOM | 3908 | O | TYR | B | 346 | 18.984 | −10.668 | 101.930 | 1.00 | 48.46 | O |
| ATOM | 3909 | CB | TYR | B | 346 | 16.111 | −11.534 | 103.109 | 1.00 | 48.65 | C |
| ATOM | 3910 | CG | TYR | B | 346 | 14.680 | −12.013 | 103.080 | 1.00 | 48.56 | C |
| ATOM | 3911 | CD1 | TYR | B | 346 | 13.627 | −11.113 | 102.897 | 1.00 | 48.61 | C |
| ATOM | 3912 | CD2 | TYR | B | 346 | 14.374 | −13.363 | 103.231 | 1.00 | 48.29 | C |
| ATOM | 3913 | CE1 | TYR | B | 346 | 12.306 | −11.550 | 102.866 | 1.00 | 48.79 | C |
| ATOM | 3914 | CE2 | TYR | B | 346 | 13.057 | −13.810 | 103.203 | 1.00 | 48.37 | C |
| ATOM | 3915 | CZ | TYR | B | 346 | 12.030 | −12.900 | 103.019 | 1.00 | 48.59 | C |
| ATOM | 3916 | OH | TYR | B | 346 | 10.727 | −13.336 | 102.987 | 1.00 | 48.67 | O |
| ATOM | 3917 | N | ARG | B | 347 | 17.529 | −8.959 | 101.727 | 1.00 | 48.11 | N |
| ATOM | 3918 | CA | ARG | B | 347 | 18.555 | −7.952 | 101.510 | 1.00 | 47.78 | C |
| ATOM | 3919 | C | ARG | B | 347 | 18.073 | −6.570 | 101.943 | 1.00 | 47.73 | C |
| ATOM | 3920 | O | ARG | B | 347 | 16.872 | −6.312 | 101.995 | 1.00 | 47.77 | O |
| ATOM | 3921 | CB | ARG | B | 347 | 18.924 | −7.948 | 100.024 | 1.00 | 47.68 | C |
| ATOM | 3922 | CG | ARG | B | 347 | 20.225 | −7.270 | 99.668 | 1.00 | 47.49 | C |
| ATOM | 3923 | CD | ARG | B | 347 | 21.421 | −8.196 | 99.701 | 1.00 | 46.20 | C |
| ATOM | 3924 | NE | ARG | B | 347 | 22.444 | −7.733 | 98.768 | 1.00 | 45.04 | N |
| ATOM | 3925 | CZ | ARG | B | 347 | 23.225 | −6.675 | 98.960 | 1.00 | 44.53 | C |
| ATOM | 3926 | NH1 | ARG | B | 347 | 23.119 | −5.947 | 100.064 | 1.00 | 44.12 | N |
| ATOM | 3927 | NH2 | ARG | B | 347 | 24.118 | −6.342 | 98.037 | 1.00 | 44.53 | N |
| ATOM | 3928 | N | ILE | B | 348 | 19.023 | −5.694 | 102.264 | 1.00 | 47.75 | N |
| ATOM | 3929 | CA | ILE | B | 348 | 18.760 | −4.270 | 102.553 | 1.00 | 47.82 | C |
| ATOM | 3930 | C | ILE | B | 348 | 17.856 | −4.050 | 103.785 | 1.00 | 47.86 | C |
| ATOM | 3931 | O | ILE | B | 348 | 16.642 | −3.868 | 103.644 | 1.00 | 48.00 | O |
| ATOM | 3932 | CB | ILE | B | 348 | 18.204 | −3.488 | 101.302 | 1.00 | 47.74 | C |
| ATOM | 3933 | CG1 | ILE | B | 348 | 18.846 | −3.965 | 99.984 | 1.00 | 47.75 | C |
| ATOM | 3934 | CG2 | ILE | B | 348 | 18.355 | −1.985 | 101.488 | 1.00 | 47.77 | C |
| ATOM | 3935 | CD1 | ILE | B | 348 | 20.356 | −3.746 | 99.858 | 1.00 | 47.38 | C |
| ATOM | 3936 | N | PRO | B | 349 | 18.452 | −4.070 | 104.993 | 1.00 | 47.78 | N |
| ATOM | 3937 | CA | PRO | B | 349 | 17.737 | −3.858 | 106.248 | 1.00 | 47.73 | C |
| ATOM | 3938 | C | PRO | B | 349 | 17.346 | −2.403 | 106.493 | 1.00 | 47.80 | C |
| ATOM | 3939 | O | PRO | B | 349 | 18.099 | −1.486 | 106.158 | 1.00 | 47.90 | O |
| ATOM | 3940 | CB | PRO | B | 349 | 18.761 | −4.284 | 107.302 | 1.00 | 47.74 | C |
| ATOM | 3941 | CG | PRO | B | 349 | 20.067 | −4.040 | 106.680 | 1.00 | 47.83 | C |
| ATOM | 3942 | CD | PRO | B | 349 | 19.886 | −4.325 | 105.222 | 1.00 | 47.76 | C |
| ATOM | 3943 | N | ALA | B | 350 | 16.173 | −2.205 | 107.083 | 1.00 | 47.79 | N |
| ATOM | 3944 | CA | ALA | B | 350 | 15.756 | −0.893 | 107.565 | 1.00 | 47.91 | C |
| ATOM | 3945 | C | ALA | B | 350 | 15.011 | −1.073 | 108.882 | 1.00 | 48.03 | C |
| ATOM | 3946 | O | ALA | B | 350 | 14.053 | −1.846 | 108.957 | 1.00 | 48.06 | O |
| ATOM | 3947 | CB | ALA | B | 350 | 14.883 | −0.190 | 106.535 | 1.00 | 47.78 | C |
| ATOM | 3948 | N | LEU | B | 351 | 15.462 | −0.367 | 109.917 | 1.00 | 48.17 | N |
| ATOM | 3949 | CA | LEU | B | 351 | 14.898 | −0.516 | 111.257 | 1.00 | 48.33 | C |
| ATOM | 3950 | C | LEU | B | 351 | 14.356 | 0.795 | 111.816 | 1.00 | 48.64 | C |
| ATOM | 3951 | O | LEU | B | 351 | 15.074 | 1.795 | 111.877 | 1.00 | 48.55 | O |
| ATOM | 3952 | CB | LEU | B | 351 | 15.941 | −1.104 | 112.216 | 1.00 | 48.26 | C |
| ATOM | 3953 | CG | LEU | B | 351 | 15.473 | −1.448 | 113.634 | 1.00 | 47.83 | C |
| ATOM | 3954 | CD1 | LEU | B | 351 | 14.732 | −2.776 | 113.654 | 1.00 | 47.56 | C |
| ATOM | 3955 | CD2 | LEU | B | 351 | 16.653 | −1.476 | 114.592 | 1.00 | 47.33 | C |
| ATOM | 3956 | N | LEU | B | 352 | 13.091 | 0.776 | 112.233 | 1.00 | 49.13 | N |
| ATOM | 3957 | CA | LEU | B | 352 | 12.450 | 1.946 | 112.837 | 1.00 | 49.45 | C |
| ATOM | 3958 | C | LEU | B | 352 | 11.702 | 1.614 | 114.130 | 1.00 | 49.82 | C |
| ATOM | 3959 | O | LEU | B | 352 | 10.928 | 0.658 | 114.178 | 1.00 | 49.94 | O |
| ATOM | 3960 | CB | LEU | B | 352 | 11.497 | 2.609 | 111.836 | 1.00 | 49.38 | C |
| ATOM | 3961 | CG | LEU | B | 352 | 10.715 | 3.858 | 112.268 | 1.00 | 49.32 | C |
| ATOM | 3962 | CD1 | LEU | B | 352 | 11.633 | 5.033 | 112.599 | 1.00 | 48.78 | C |
| ATOM | 3963 | CD2 | LEU | B | 352 | 9.713 | 4.247 | 111.192 | 1.00 | 49.34 | C |
| ATOM | 3964 | N | LYS | B | 353 | 11.949 | 2.407 | 115.172 | 1.00 | 50.18 | N |
| ATOM | 3965 | CA | LYS | B | 353 | 11.132 | 2.385 | 116.379 | 1.00 | 50.39 | C |
| ATOM | 3966 | C | LYS | B | 353 | 10.185 | 3.583 | 116.339 | 1.00 | 50.51 | C |
| ATOM | 3967 | O | LYS | B | 353 | 10.613 | 4.710 | 116.071 | 1.00 | 50.41 | O |
| ATOM | 3968 | CB | LYS | B | 353 | 12.001 | 2.406 | 117.641 | 1.00 | 50.56 | C |
| ATOM | 3969 | CG | LYS | B | 353 | 11.236 | 2.113 | 118.934 | 1.00 | 50.89 | C |
| ATOM | 3970 | CD | LYS | B | 353 | 12.098 | 1.369 | 119.957 | 1.00 | 51.58 | C |
| ATOM | 3971 | CE | LYS | B | 353 | 11.347 | 1.160 | 121.278 | 1.00 | 51.78 | C |
| ATOM | 3972 | NZ | LYS | B | 353 | 11.834 | −0.017 | 122.058 | 1.00 | 51.32 | N |
| ATOM | 3973 | N | THR | B | 354 | 8.904 | 3.328 | 116.604 | 1.00 | 50.68 | N |
| ATOM | 3974 | CA | THR | B | 354 | 7.846 | 4.329 | 116.426 | 1.00 | 50.85 | C |
| ATOM | 3975 | C | THR | B | 354 | 7.460 | 5.055 | 117.724 | 1.00 | 51.03 | C |
| ATOM | 3976 | O | THR | B | 354 | 8.191 | 4.999 | 118.714 | 1.00 | 51.04 | O |
| ATOM | 3977 | CB | THR | B | 354 | 6.593 | 3.713 | 115.757 | 1.00 | 50.88 | C |
| ATOM | 3978 | OG1 | THR | B | 354 | 5.856 | 2.941 | 116.712 | 1.00 | 50.76 | O |
| ATOM | 3979 | CG2 | THR | B | 354 | 6.993 | 2.828 | 114.585 | 1.00 | 50.77 | C |
| ATOM | 3980 | N | ASP | B | 355 | 6.318 | 5.745 | 117.703 | 1.00 | 51.24 | N |
| ATOM | 3981 | CA | ASP | B | 355 | 5.854 | 6.521 | 118.855 | 1.00 | 51.56 | C |
| ATOM | 3982 | C | ASP | B | 355 | 5.273 | 5.630 | 119.947 | 1.00 | 51.73 | C |
| ATOM | 3983 | O | ASP | B | 355 | 5.427 | 5.915 | 121.134 | 1.00 | 51.90 | O |
| ATOM | 3984 | CB | ASP | B | 355 | 4.834 | 7.589 | 118.431 | 1.00 | 51.47 | C |
| ATOM | 3985 | CG | ASP | B | 355 | 3.563 | 6.993 | 117.843 | 1.00 | 51.63 | C |
| ATOM | 3986 | OD1 | ASP | B | 355 | 3.662 | 6.076 | 117.000 | 1.00 | 51.93 | O |
| ATOM | 3987 | OD2 | ASP | B | 355 | 2.462 | 7.450 | 118.221 | 1.00 | 51.46 | O |
| ATOM | 3988 | N | LYS | B | 356 | 4.615 | 4.549 | 119.532 | 1.00 | 51.87 | N |
| ATOM | 3989 | CA | LYS | B | 356 | 4.010 | 3.592 | 120.454 | 1.00 | 51.97 | C |
| ATOM | 3990 | C | LYS | B | 356 | 5.036 | 2.600 | 121.012 | 1.00 | 51.87 | C |
| ATOM | 3991 | O | LYS | B | 356 | 4.697 | 1.744 | 121.839 | 1.00 | 51.95 | O |
| ATOM | 3992 | CB | LYS | B | 356 | 2.865 | 2.844 | 119.761 | 1.00 | 52.13 | C |
| ATOM | 3993 | CG | LYS | B | 356 | 1.680 | 3.724 | 119.394 | 1.00 | 52.79 | C |
| ATOM | 3994 | CD | LYS | B | 356 | 0.646 | 2.960 | 118.585 | 1.00 | 53.70 | C |
| ATOM | 3995 | CE | LYS | B | 356 | −0.595 | 3.808 | 118.347 | 1.00 | 53.91 | C |
| ATOM | 3996 | NZ | LYS | B | 356 | −1.516 | 3.160 | 117.370 | 1.00 | 54.11 | N |
| ATOM | 3997 | N | GLY | B | 357 | 6.283 | 2.721 | 120.557 | 1.00 | 51.67 | N |
| ATOM | 3998 | CA | GLY | B | 357 | 7.374 | 1.846 | 121.000 | 1.00 | 51.30 | C |
| ATOM | 3999 | C | GLY | B | 357 | 7.657 | 0.691 | 120.053 | 1.00 | 51.04 | C |
| ATOM | 4000 | O | GLY | B | 357 | 8.680 | 0.013 | 120.181 | 1.00 | 51.03 | O |
| ATOM | 4001 | N | THR | B | 358 | 6.747 | 0.477 | 119.104 | 1.00 | 50.66 | N |
| ATOM | 4002 | CA | THR | B | 358 | 6.838 | −0.600 | 118.115 | 1.00 | 50.36 | C |
| ATOM | 4003 | C | THR | B | 358 | 8.142 | −0.573 | 117.311 | 1.00 | 50.21 | C |
| ATOM | 4004 | O | THR | B | 358 | 8.666 | 0.496 | 116.996 | 1.00 | 50.14 | O |
| ATOM | 4005 | CB | THR | B | 358 | 5.661 | −0.524 | 117.117 | 1.00 | 50.36 | C |
| ATOM | 4006 | OG1 | THR | B | 358 | 4.453 | −0.185 | 117.812 | 1.00 | 50.23 | O |
| ATOM | 4007 | CG2 | THR | B | 358 | 5.475 | −1.845 | 116.380 | 1.00 | 50.43 | C |
| ATOM | 4008 | N | LEU | B | 359 | 8.658 | −1.757 | 116.989 | 1.00 | 50.00 | N |
| ATOM | 4009 | CA | LEU | B | 359 | 9.769 | −1.881 | 116.048 | 1.00 | 49.71 | C |
| ATOM | 4010 | C | LEU | B | 359 | 9.287 | −2.383 | 114.685 | 1.00 | 49.41 | C |
| ATOM | 4011 | O | LEU | B | 359 | 8.510 | −3.337 | 114.596 | 1.00 | 49.38 | O |
| ATOM | 4012 | CB | LEU | B | 359 | 10.876 | −2.788 | 116.598 | 1.00 | 49.68 | C |
| ATOM | 4013 | CG | LEU | B | 359 | 11.779 | −2.226 | 117.704 | 1.00 | 49.93 | C |
| ATOM | 4014 | CD1 | LEU | B | 359 | 12.587 | −3.342 | 118.350 | 1.00 | 49.79 | C |
| ATOM | 4015 | CD2 | LEU | B | 359 | 12.705 | −1.136 | 117.180 | 1.00 | 49.93 | C |
| ATOM | 4016 | N | ILE | B | 360 | 9.744 | −1.709 | 113.633 | 1.00 | 49.00 | N |
| ATOM | 4017 | CA | ILE | B | 360 | 9.483 | −2.116 | 112.259 | 1.00 | 48.46 | C |
| ATOM | 4018 | C | ILE | B | 360 | 10.804 | −2.504 | 111.613 | 1.00 | 48.12 | C |
| ATOM | 4019 | O | ILE | B | 360 | 11.776 | −1.751 | 111.670 | 1.00 | 47.96 | O |
| ATOM | 4020 | CB | ILE | B | 360 | 8.825 | −0.981 | 111.437 | 1.00 | 48.47 | C |
| ATOM | 4021 | CG1 | ILE | B | 360 | 7.496 | −0.560 | 112.070 | 1.00 | 48.47 | C |
| ATOM | 4022 | CG2 | ILE | B | 360 | 8.616 | −1.414 | 109.987 | 1.00 | 48.29 | C |
| ATOM | 4023 | CD1 | ILE | B | 360 | 6.952 | 0.755 | 111.556 | 1.00 | 48.37 | C |
| ATOM | 4024 | N | ALA | B | 361 | 10.835 | −3.694 | 111.022 | 1.00 | 47.76 | N |
| ATOM | 4025 | CA | ALA | B | 361 | 11.976 | −4.133 | 110.232 | 1.00 | 47.42 | C |
| ATOM | 4026 | C | ALA | B | 361 | 11.540 | −4.331 | 108.782 | 1.00 | 47.15 | C |
| ATOM | 4027 | O | ALA | B | 361 | 10.560 | −5.027 | 108.512 | 1.00 | 47.05 | O |
| ATOM | 4028 | CB | ALA | B | 361 | 12.569 | −5.411 | 110.806 | 1.00 | 47.35 | C |
| ATOM | 4029 | N | GLY | B | 362 | 12.265 | −3.699 | 107.861 | 1.00 | 46.87 | N |
| ATOM | 4030 | CA | GLY | B | 362 | 11.951 | −3.760 | 106.432 | 1.00 | 46.49 | C |
| ATOM | 4031 | C | GLY | B | 362 | 13.077 | −4.347 | 105.604 | 1.00 | 46.16 | C |
| ATOM | 4032 | O | GLY | B | 362 | 14.254 | −4.185 | 105.937 | 1.00 | 46.11 | O |
| ATOM | 4033 | N | ALA | B | 363 | 12.714 | −5.022 | 104.516 | 1.00 | 45.89 | N |
| ATOM | 4034 | CA | ALA | B | 363 | 13.688 | −5.739 | 103.697 | 1.00 | 45.72 | C |
| ATOM | 4035 | C | ALA | B | 363 | 13.297 | −5.836 | 102.229 | 1.00 | 45.65 | C |
| ATOM | 4036 | O | ALA | B | 363 | 12.127 | −5.672 | 101.871 | 1.00 | 45.58 | O |
| ATOM | 4037 | CB | ALA | B | 363 | 13.909 | −7.136 | 104.262 | 1.00 | 45.59 | C |
| ATOM | 4038 | N | ASP | B | 364 | 14.298 | −6.099 | 101.391 | 1.00 | 45.71 | N |
| ATOM | 4039 | CA | ASP | B | 364 | 14.088 | −6.549 | 100.018 | 1.00 | 45.79 | C |
| ATOM | 4040 | C | ASP | B | 364 | 13.752 | −8.041 | 100.047 | 1.00 | 45.76 | C |
| ATOM | 4041 | O | ASP | B | 364 | 14.560 | −8.859 | 100.491 | 1.00 | 45.77 | O |
| ATOM | 4042 | CB | ASP | B | 364 | 15.350 | −6.334 | 99.167 | 1.00 | 45.73 | C |
| ATOM | 4043 | CG | ASP | B | 364 | 15.442 | −4.935 | 98.567 | 1.00 | 46.25 | C |
| ATOM | 4044 | OD1 | ASP | B | 364 | 15.251 | −3.933 | 99.295 | 1.00 | 46.69 | O |
| ATOM | 4045 | OD2 | ASP | B | 364 | 15.736 | −4.841 | 97.355 | 1.00 | 46.25 | O |
| ATOM | 4046 | N | GLU | B | 365 | 12.554 | −8.388 | 99.590 | 1.00 | 45.69 | N |
| ATOM | 4047 | CA | GLU | B | 365 | 12.169 | −9.784 | 99.426 | 1.00 | 45.56 | C |
| ATOM | 4048 | C | GLU | B | 365 | 12.576 | −10.256 | 98.032 | 1.00 | 45.46 | C |
| ATOM | 4049 | O | GLU | B | 365 | 11.749 | −10.322 | 97.116 | 1.00 | 45.56 | O |
| ATOM | 4050 | CB | GLU | B | 365 | 10.667 | −9.942 | 99.642 | 1.00 | 45.66 | C |
| ATOM | 4051 | CG | GLU | B | 365 | 10.188 | −11.372 | 99.806 | 1.00 | 45.78 | C |
| ATOM | 4052 | CD | GLU | B | 365 | 8.854 | −11.437 | 100.520 | 1.00 | 45.95 | C |
| ATOM | 4053 | OE1 | GLU | B | 365 | 8.848 | −11.433 | 101.772 | 1.00 | 45.59 | O |
| ATOM | 4054 | OE2 | GLU | B | 365 | 7.815 | −11.487 | 99.829 | 1.00 | 45.80 | O |
| ATOM | 4055 | N | ARG | B | 366 | 13.861 | −10.574 | 97.885 | 1.00 | 45.24 | N |
| ATOM | 4056 | CA | ARG | B | 366 | 14.435 | −10.965 | 96.597 | 1.00 | 44.93 | C |
| ATOM | 4057 | C | ARG | B | 366 | 14.233 | −12.455 | 96.330 | 1.00 | 45.00 | C |
| ATOM | 4058 | O | ARG | B | 366 | 15.082 | −13.282 | 96.661 | 1.00 | 45.01 | O |
| ATOM | 4059 | CB | ARG | B | 366 | 15.919 | −10.582 | 96.532 | 1.00 | 44.82 | C |
| ATOM | 4060 | CG | ARG | B | 366 | 16.170 | −9.092 | 96.693 | 1.00 | 43.87 | C |
| ATOM | 4061 | CD | ARG | B | 366 | 17.638 | −8.746 | 96.586 | 1.00 | 42.36 | C |
| ATOM | 4062 | NE | ARG | B | 366 | 17.862 | −7.315 | 96.781 | 1.00 | 40.97 | N |
| ATOM | 4063 | CZ | ARG | B | 366 | 19.019 | −6.694 | 96.571 | 1.00 | 40.40 | C |
| ATOM | 4064 | NH1 | ARG | B | 366 | 20.085 | −7.370 | 96.162 | 1.00 | 40.04 | N |
| ATOM | 4065 | NH2 | ARG | B | 366 | 19.113 | −5.389 | 96.779 | 1.00 | 40.30 | N |
| ATOM | 4066 | N | ARG | B | 367 | 13.101 | −12.772 | 95.706 | 1.00 | 45.12 | N |
| ATOM | 4067 | CA | ARG | B | 367 | 12.622 | −14.147 | 95.558 | 1.00 | 45.09 | C |
| ATOM | 4068 | C | ARG | B | 367 | 13.529 | −15.083 | 94.759 | 1.00 | 44.98 | C |
| ATOM | 4069 | O | ARG | B | 367 | 13.737 | −16.230 | 95.156 | 1.00 | 45.15 | O |
| ATOM | 4070 | CB | ARG | B | 367 | 11.209 | −14.157 | 94.959 | 1.00 | 45.16 | C |
| ATOM | 4071 | CG | ARG | B | 367 | 10.533 | −15.517 | 95.025 | 1.00 | 45.58 | C |
| ATOM | 4072 | CD | ARG | B | 367 | 9.034 | −15.434 | 94.802 | 1.00 | 46.25 | C |
| ATOM | 4073 | NE | ARG | B | 367 | 8.670 | −15.472 | 93.387 | 1.00 | 47.11 | N |
| ATOM | 4074 | CZ | ARG | B | 367 | 8.644 | −16.571 | 92.633 | 1.00 | 47.46 | C |
| ATOM | 4075 | NH1 | ARG | B | 367 | 8.983 | −17.753 | 93.142 | 1.00 | 47.77 | N |
| ATOM | 4076 | NH2 | ARG | B | 367 | 8.285 | −16.486 | 91.358 | 1.00 | 47.42 | N |
| ATOM | 4077 | N | LEU | B | 368 | 14.068 | −14.599 | 93.646 | 1.00 | 44.89 | N |
| ATOM | 4078 | CA | LEU | B | 368 | 14.708 | −15.486 | 92.671 | 1.00 | 44.77 | C |
| ATOM | 4079 | C | LEU | B | 368 | 16.237 | −15.579 | 92.741 | 1.00 | 44.73 | C |
| ATOM | 4080 | O | LEU | B | 368 | 16.812 | −16.589 | 92.334 | 1.00 | 44.75 | O |
| ATOM | 4081 | CB | LEU | B | 368 | 14.229 | −15.148 | 91.255 | 1.00 | 44.76 | C |
| ATOM | 4082 | CG | LEU | B | 368 | 12.786 | −15.579 | 90.963 | 1.00 | 44.78 | C |
| ATOM | 4083 | CD1 | LEU | B | 368 | 12.110 | −14.628 | 89.985 | 1.00 | 45.16 | C |
| ATOM | 4084 | CD2 | LEU | B | 368 | 12.719 | −17.024 | 90.463 | 1.00 | 45.06 | C |
| ATOM | 4085 | N | HIS | B | 369 | 16.878 | −14.524 | 93.244 | 1.00 | 44.65 | N |
| ATOM | 4086 | CA | HIS | B | 369 | 18.330 | −14.482 | 93.457 | 1.00 | 44.62 | C |
| ATOM | 4087 | C | HIS | B | 369 | 18.676 | −13.314 | 94.379 | 1.00 | 44.62 | C |
| ATOM | 4088 | O | HIS | B | 369 | 17.784 | −12.595 | 94.830 | 1.00 | 44.69 | O |
| ATOM | 4089 | CB | HIS | B | 369 | 19.088 | −14.358 | 92.129 | 1.00 | 44.66 | C |
| ATOM | 4090 | CG | HIS | B | 369 | 18.498 | −13.355 | 91.189 | 1.00 | 44.74 | C |
| ATOM | 4091 | ND1 | HIS | B | 369 | 18.651 | −11.995 | 91.358 | 1.00 | 44.68 | N |
| ATOM | 4092 | CD2 | HIS | B | 369 | 17.746 | −13.513 | 90.074 | 1.00 | 44.34 | C |
| ATOM | 4093 | CE1 | HIS | B | 369 | 18.021 | −11.360 | 90.387 | 1.00 | 44.02 | C |
| ATOM | 4094 | NE2 | HIS | B | 369 | 17.464 | −12.258 | 89.595 | 1.00 | 44.13 | N |
| ATOM | 4095 | N | SER | B | 370 | 19.963 | −13.130 | 94.660 | 1.00 | 44.71 | N |
| ATOM | 4096 | CA | SER | B | 370 | 20.413 | −12.031 | 95.516 | 1.00 | 44.85 | C |
| ATOM | 4097 | C | SER | B | 370 | 21.070 | −10.874 | 94.749 | 1.00 | 44.90 | C |
| ATOM | 4098 | O | SER | B | 370 | 21.878 | −10.129 | 95.308 | 1.00 | 45.06 | O |
| ATOM | 4099 | CB | SER | B | 370 | 21.339 | −12.551 | 96.626 | 1.00 | 44.88 | C |
| ATOM | 4100 | OG | SER | B | 370 | 22.632 | −12.845 | 96.138 | 1.00 | 44.99 | O |
| ATOM | 4101 | N | SER | B | 371 | 20.717 | −10.716 | 93.475 | 1.00 | 44.98 | N |
| ATOM | 4102 | CA | SER | B | 371 | 21.215 | −9.588 | 92.688 | 1.00 | 45.00 | C |
| ATOM | 4103 | C | SER | B | 371 | 20.292 | −8.371 | 92.790 | 1.00 | 44.99 | C |
| ATOM | 4104 | O | SER | B | 371 | 19.149 | −8.479 | 93.239 | 1.00 | 44.97 | O |
| ATOM | 4105 | CB | SER | B | 371 | 21.434 | −9.994 | 91.232 | 1.00 | 45.05 | C |
| ATOM | 4106 | OG | SER | B | 371 | 22.617 | −10.761 | 91.102 | 1.00 | 45.18 | O |
| ATOM | 4107 | N | ASP | B | 372 | 20.797 | −7.215 | 92.369 | 1.00 | 44.92 | N |
| ATOM | 4108 | CA | ASP | B | 372 | 20.069 | −5.954 | 92.502 | 1.00 | 44.79 | C |
| ATOM | 4109 | C | ASP | B | 372 | 18.957 | −5.768 | 91.451 | 1.00 | 44.74 | C |
| ATOM | 4110 | O | ASP | B | 372 | 18.801 | −4.683 | 90.883 | 1.00 | 44.57 | O |
| ATOM | 4111 | CB | ASP | B | 372 | 21.055 | −4.776 | 92.495 | 1.00 | 44.77 | C |
| ATOM | 4112 | CG | ASP | B | 372 | 22.114 | −4.888 | 93.586 | 1.00 | 44.78 | C |
| ATOM | 4113 | OD1 | ASP | B | 372 | 21.822 | −5.457 | 94.659 | 1.00 | 44.27 | O |
| ATOM | 4114 | OD2 | ASP | B | 372 | 23.244 | −4.401 | 93.371 | 1.00 | 45.46 | O |
| ATOM | 4115 | N | TRP | B | 373 | 18.189 | −6.835 | 91.213 | 1.00 | 44.81 | N |
| ATOM | 4116 | CA | TRP | B | 373 | 17.060 | −6.835 | 90.269 | 1.00 | 44.96 | C |
| ATOM | 4117 | C | TRP | B | 373 | 16.212 | −8.111 | 90.397 | 1.00 | 45.10 | C |
| ATOM | 4118 | O | TRP | B | 373 | 16.465 | −8.946 | 91.265 | 1.00 | 45.06 | O |
| ATOM | 4119 | CB | TRP | B | 373 | 17.554 | −6.677 | 88.826 | 1.00 | 44.83 | C |
| ATOM | 4120 | CG | TRP | B | 373 | 18.462 | −7.785 | 88.378 | 1.00 | 44.82 | C |
| ATOM | 4121 | CD1 | TRP | B | 373 | 18.090 | −9.028 | 87.944 | 1.00 | 44.62 | C |
| ATOM | 4122 | CD2 | TRP | B | 373 | 19.891 | −7.750 | 88.317 | 1.00 | 44.65 | C |
| ATOM | 4123 | NE1 | TRP | B | 373 | 19.199 | −9.769 | 87.621 | 1.00 | 44.43 | N |
| ATOM | 4124 | CE2 | TRP | B | 373 | 20.318 | −9.010 | 87.838 | 1.00 | 44.62 | C |
| ATOM | 4125 | CE3 | TRP | B | 373 | 20.853 | −6.777 | 88.618 | 1.00 | 44.70 | C |
| ATOM | 4126 | CZ2 | TRP | B | 373 | 21.667 | −9.322 | 87.652 | 1.00 | 44.50 | C |
| ATOM | 4127 | CZ3 | TRP | B | 373 | 22.196 | −7.091 | 88.437 | 1.00 | 44.79 | C |
| ATOM | 4128 | CH2 | TRP | B | 373 | 22.588 | −8.354 | 87.957 | 1.00 | 44.65 | C |
| ATOM | 4129 | N | GLY | B | 374 | 15.218 | −8.257 | 89.524 | 1.00 | 45.41 | N |
| ATOM | 4130 | CA | GLY | B | 374 | 14.335 | −9.429 | 89.527 | 1.00 | 46.01 | C |
| ATOM | 4131 | C | GLY | B | 374 | 12.984 | −9.168 | 90.169 | 1.00 | 46.28 | C |
| ATOM | 4132 | O | GLY | B | 374 | 12.479 | −8.046 | 90.126 | 1.00 | 46.37 | O |
| ATOM | 4133 | N | ASP | B | 375 | 12.391 | −10.211 | 90.749 | 1.00 | 46.67 | N |
| ATOM | 4134 | CA | ASP | B | 375 | 11.153 | −10.068 | 91.510 | 1.00 | 46.93 | C |
| ATOM | 4135 | C | ASP | B | 375 | 11.481 | −9.769 | 92.961 | 1.00 | 47.23 | C |
| ATOM | 4136 | O | ASP | B | 375 | 11.916 | −10.651 | 93.709 | 1.00 | 47.40 | O |
| ATOM | 4137 | CB | ASP | B | 375 | 10.274 | −11.319 | 91.409 | 1.00 | 46.99 | C |
| ATOM | 4138 | CG | ASP | B | 375 | 9.056 | −11.260 | 92.329 | 1.00 | 46.98 | C |
| ATOM | 4139 | OD1 | ASP | B | 375 | 8.441 | −10.180 | 92.468 | 1.00 | 47.12 | O |
| ATOM | 4140 | OD2 | ASP | B | 375 | 8.713 | −12.303 | 92.916 | 1.00 | 46.95 | O |
| ATOM | 4141 | N | ILE | B | 376 | 11.280 | −8.510 | 93.340 | 1.00 | 47.52 | N |
| ATOM | 4142 | CA | ILE | B | 376 | 11.541 | −8.043 | 94.695 | 1.00 | 47.73 | C |
| ATOM | 4143 | C | ILE | B | 376 | 10.289 | −7.381 | 95.266 | 1.00 | 48.20 | C |
| ATOM | 4144 | O | ILE | B | 376 | 9.669 | −6.534 | 94.613 | 1.00 | 48.20 | O |
| ATOM | 4145 | CB | ILE | B | 376 | 12.717 | −7.037 | 94.746 | 1.00 | 47.53 | C |
| ATOM | 4146 | CG1 | ILE | B | 376 | 13.933 | −7.575 | 93.984 | 1.00 | 47.62 | C |
| ATOM | 4147 | CG2 | ILE | B | 376 | 13.081 | −6.710 | 96.192 | 1.00 | 47.41 | C |
| ATOM | 4148 | CD1 | ILE | B | 376 | 15.052 | −6.559 | 93.780 | 1.00 | 47.57 | C |
| ATOM | 4149 | N | GLY | B | 377 | 9.924 | −7.780 | 96.483 | 1.00 | 48.57 | N |
| ATOM | 4150 | CA | GLY | B | 377 | 8.837 | −7.141 | 97.213 | 1.00 | 49.24 | C |
| ATOM | 4151 | C | GLY | B | 377 | 9.353 | −6.303 | 98.365 | 1.00 | 49.81 | C |
| ATOM | 4152 | O | GLY | B | 377 | 10.409 | −6.591 | 98.932 | 1.00 | 49.81 | O |
| ATOM | 4153 | N | MET | B | 378 | 8.621 | −5.247 | 98.697 | 1.00 | 50.50 | N |
| ATOM | 4154 | CA | MET | B | 378 | 8.917 | −4.466 | 99.889 | 1.00 | 51.26 | C |
| ATOM | 4155 | C | MET | B | 378 | 8.093 | −5.069 | 101.023 | 1.00 | 52.00 | C |
| ATOM | 4156 | O | MET | B | 378 | 6.862 | −5.055 | 100.973 | 1.00 | 52.07 | O |
| ATOM | 4157 | CB | MET | B | 378 | 8.576 | −2.985 | 99.688 | 1.00 | 51.01 | C |
| ATOM | 4158 | CG | MET | B | 378 | 9.220 | −2.314 | 98.468 | 1.00 | 50.64 | C |
| ATOM | 4159 | SD | MET | B | 378 | 10.994 | −1.972 | 98.586 | 1.00 | 49.65 | S |
| ATOM | 4160 | CE | MET | B | 378 | 11.700 | −3.429 | 97.816 | 1.00 | 49.55 | C |
| ATOM | 4161 | N | VAL | B | 379 | 8.773 | −5.623 | 102.024 | 1.00 | 52.85 | N |
| ATOM | 4162 | CA | VAL | B | 379 | 8.090 | −6.301 | 103.129 | 1.00 | 53.77 | C |
| ATOM | 4163 | C | VAL | B | 379 | 8.565 | −5.832 | 104.500 | 1.00 | 54.44 | C |
| ATOM | 4164 | O | VAL | B | 379 | 9.725 | −5.448 | 104.673 | 1.00 | 54.47 | O |
| ATOM | 4165 | CB | VAL | B | 379 | 8.210 | −7.853 | 103.047 | 1.00 | 53.67 | C |
| ATOM | 4166 | CG1 | VAL | B | 379 | 7.345 | −8.411 | 101.917 | 1.00 | 53.82 | C |
| ATOM | 4167 | CG2 | VAL | B | 379 | 9.668 | −8.290 | 102.899 | 1.00 | 53.59 | C |
| ATOM | 4168 | N | ILE | B | 380 | 7.651 | −5.863 | 105.468 | 1.00 | 55.29 | N |
| ATOM | 4169 | CA | ILE | B | 380 | 7.976 | −5.541 | 106.855 | 1.00 | 56.03 | C |
| ATOM | 4170 | C | ILE | B | 380 | 7.469 | −6.591 | 107.846 | 1.00 | 56.52 | C |
| ATOM | 4171 | O | ILE | B | 380 | 6.537 | −7.345 | 107.554 | 1.00 | 56.49 | O |
| ATOM | 4172 | CB | ILE | B | 380 | 7.466 | −4.127 | 107.275 | 1.00 | 56.02 | C |
| ATOM | 4173 | CG1 | ILE | B | 380 | 5.968 | −3.957 | 106.984 | 1.00 | 56.35 | C |
| ATOM | 4174 | CG2 | ILE | B | 380 | 8.289 | −3.025 | 106.610 | 1.00 | 56.03 | C |
| ATOM | 4175 | CD1 | ILE | B | 380 | 5.061 | −4.298 | 108.159 | 1.00 | 56.33 | C |
| ATOM | 4176 | N | ARG | B | 381 | 8.109 | −6.632 | 109.011 | 1.00 | 57.23 | N |
| ATOM | 4177 | CA | ARG | B | 381 | 7.662 | −7.442 | 110.140 | 1.00 | 57.82 | C |
| ATOM | 4178 | C | ARG | B | 381 | 7.702 | −6.591 | 111.408 | 1.00 | 58.23 | C |
| ATOM | 4179 | O | ARG | B | 381 | 8.651 | −5.832 | 111.627 | 1.00 | 58.21 | O |
| ATOM | 4180 | CB | ARG | B | 381 | 8.520 | −8.701 | 110.288 | 1.00 | 57.70 | C |
| ATOM | 4181 | CG | ARG | B | 381 | 8.238 | −9.771 | 109.233 | 1.00 | 57.88 | C |
| ATOM | 4182 | CD | ARG | B | 381 | 8.938 | −11.087 | 109.549 | 1.00 | 58.00 | C |
| ATOM | 4183 | NE | ARG | B | 381 | 8.420 | −11.712 | 110.766 | 1.00 | 57.96 | N |
| ATOM | 4184 | CZ | ARG | B | 381 | 8.975 | −12.754 | 111.382 | 1.00 | 57.98 | C |
| ATOM | 4185 | NH1 | ARG | B | 381 | 10.083 | −13.310 | 110.907 | 1.00 | 57.46 | N |
| ATOM | 4186 | NH2 | ARG | B | 381 | 8.418 | −13.241 | 112.483 | 1.00 | 58.15 | N |
| ATOM | 4187 | N | ARG | B | 382 | 6.666 | −6.725 | 112.234 | 1.00 | 58.83 | N |
| ATOM | 4188 | CA | ARG | B | 382 | 6.459 | −5.837 | 113.379 | 1.00 | 59.30 | C |
| ATOM | 4189 | C | ARG | B | 382 | 6.782 | −6.474 | 114.729 | 1.00 | 59.69 | C |
| ATOM | 4190 | O | ARG | B | 382 | 6.727 | −7.696 | 114.880 | 1.00 | 59.82 | O |
| ATOM | 4191 | CB | ARG | B | 382 | 5.018 | −5.326 | 113.382 | 1.00 | 59.28 | C |
| ATOM | 4192 | CG | ARG | B | 382 | 4.686 | −4.378 | 112.237 | 1.00 | 59.41 | C |
| ATOM | 4193 | CD | ARG | B | 382 | 3.249 | −3.879 | 112.289 | 1.00 | 59.34 | C |
| ATOM | 4194 | NE | ARG | B | 382 | 3.007 | −2.992 | 113.425 | 1.00 | 59.66 | N |
| ATOM | 4195 | CZ | ARG | B | 382 | 2.445 | −3.370 | 114.572 | 1.00 | 60.05 | C |
| ATOM | 4196 | NH1 | ARG | B | 382 | 2.056 | −4.629 | 114.752 | 1.00 | 59.80 | N |
| ATOM | 4197 | NH2 | ARG | B | 382 | 2.268 | −2.486 | 115.545 | 1.00 | 59.89 | N |
| ATOM | 4198 | N | SER | B | 383 | 7.117 | −5.634 | 115.708 | 1.00 | 60.20 | N |
| ATOM | 4199 | CA | SER | B | 383 | 7.337 | −6.086 | 117.084 | 1.00 | 60.66 | C |
| ATOM | 4200 | C | SER | B | 383 | 6.814 | −5.083 | 118.104 | 1.00 | 60.93 | C |
| ATOM | 4201 | O | SER | B | 383 | 7.239 | −3.926 | 118.124 | 1.00 | 60.94 | O |
| ATOM | 4202 | CB | SER | B | 383 | 8.816 | −6.362 | 117.347 | 1.00 | 60.65 | C |
| ATOM | 4203 | OG | SER | B | 383 | 9.017 | −6.788 | 118.681 | 1.00 | 60.84 | O |
| ATOM | 4204 | N | GLU | B | 384 | 5.895 | −5.546 | 118.948 | 1.00 | 61.31 | N |
| ATOM | 4205 | CA | GLU | B | 384 | 5.325 | −4.737 | 120.022 | 1.00 | 61.55 | C |
| ATOM | 4206 | C | GLU | B | 384 | 6.128 | −4.961 | 121.295 | 1.00 | 61.62 | C |
| ATOM | 4207 | O | GLU | B | 384 | 6.102 | −4.138 | 122.209 | 1.00 | 61.58 | O |
| ATOM | 4208 | CB | GLU | B | 384 | 3.857 | −5.115 | 120.279 | 1.00 | 61.63 | C |
| ATOM | 4209 | CG | GLU | B | 384 | 2.995 | −5.340 | 119.033 | 1.00 | 61.92 | C |
| ATOM | 4210 | CD | GLU | B | 384 | 3.219 | −6.703 | 118.384 | 1.00 | 62.54 | C |
| ATOM | 4211 | OE1 | GLU | B | 384 | 3.764 | −7.613 | 119.049 | 1.00 | 62.91 | O |
| ATOM | 4212 | OE2 | GLU | B | 384 | 2.852 | −6.866 | 117.201 | 1.00 | 62.79 | O |
| ATOM | 4213 | N | ASP | B | 385 | 6.849 | −6.079 | 121.334 | 1.00 | 61.85 | N |
| ATOM | 4214 | CA | ASP | B | 385 | 7.555 | −6.523 | 122.535 | 1.00 | 62.15 | C |
| ATOM | 4215 | C | ASP | B | 385 | 9.067 | −6.260 | 122.500 | 1.00 | 62.19 | C |
| ATOM | 4216 | O | ASP | B | 385 | 9.860 | −7.099 | 122.941 | 1.00 | 62.23 | O |
| ATOM | 4217 | CB | ASP | B | 385 | 7.263 | −8.012 | 122.802 | 1.00 | 62.24 | C |
| ATOM | 4218 | CG | ASP | B | 385 | 7.516 | −8.898 | 121.582 | 1.00 | 62.66 | C |
| ATOM | 4219 | OD1 | ASP | B | 385 | 8.534 | −8.699 | 120.881 | 1.00 | 63.07 | O |
| ATOM | 4220 | OD2 | ASP | B | 385 | 6.696 | −9.807 | 121.329 | 1.00 | 62.71 | O |
| ATOM | 4221 | N | ASN | B | 386 | 9.452 | −5.092 | 121.979 | 1.00 | 62.23 | N |
| ATOM | 4222 | CA | ASN | B | 386 | 10.861 | −4.659 | 121.909 | 1.00 | 62.19 | C |
| ATOM | 4223 | C | ASN | B | 386 | 11.814 | −5.638 | 121.193 | 1.00 | 62.10 | C |
| ATOM | 4224 | O | ASN | B | 386 | 13.010 | −5.703 | 121.499 | 1.00 | 62.04 | O |
| ATOM | 4225 | CB | ASN | B | 386 | 11.388 | −4.289 | 123.312 | 1.00 | 62.17 | C |
| ATOM | 4226 | CG | ASN | B | 386 | 12.706 | −3.518 | 123.267 | 1.00 | 62.21 | C |
| ATOM | 4227 | OD1 | ASN | B | 386 | 13.022 | −2.851 | 122.278 | 1.00 | 62.54 | O |
| ATOM | 4228 | ND2 | ASN | B | 386 | 13.480 | −3.611 | 124.343 | 1.00 | 61.54 | N |
| ATOM | 4229 | N | GLY | B | 387 | 11.280 | −6.391 | 120.236 | 1.00 | 62.02 | N |
| ATOM | 4230 | CA | GLY | B | 387 | 12.087 | −7.322 | 119.454 | 1.00 | 62.00 | C |
| ATOM | 4231 | C | GLY | B | 387 | 12.296 | −8.676 | 120.104 | 1.00 | 62.01 | C |
| ATOM | 4232 | O | GLY | B | 387 | 13.271 | −9.366 | 119.804 | 1.00 | 62.00 | O |
| ATOM | 4233 | N | LYS | B | 388 | 11.382 | −9.052 | 120.996 | 1.00 | 62.00 | N |
| ATOM | 4234 | CA | LYS | B | 388 | 11.386 | −10.380 | 121.603 | 1.00 | 62.00 | C |
| ATOM | 4235 | C | LYS | B | 388 | 10.924 | −11.389 | 120.549 | 1.00 | 61.88 | C |
| ATOM | 4236 | O | LYS | B | 388 | 11.704 | −12.239 | 120.115 | 1.00 | 61.91 | O |
| ATOM | 4237 | CB | LYS | B | 388 | 10.486 | −10.398 | 122.848 | 1.00 | 62.09 | C |
| ATOM | 4238 | CG | LYS | B | 388 | 10.386 | −11.732 | 123.587 | 1.00 | 62.24 | C |
| ATOM | 4239 | CD | LYS | B | 388 | 9.306 | −11.670 | 124.666 | 1.00 | 62.14 | C |
| ATOM | 4240 | CE | LYS | B | 388 | 8.913 | −13.053 | 125.166 | 1.00 | 62.55 | C |
| ATOM | 4241 | NZ | LYS | B | 388 | 9.897 | −13.611 | 126.137 | 1.00 | 62.98 | N |
| ATOM | 4242 | N | THR | B | 389 | 9.662 | −11.274 | 120.137 | 1.00 | 61.73 | N |
| ATOM | 4243 | CA | THR | B | 389 | 9.110 | −12.050 | 119.022 | 1.00 | 61.54 | C |
| ATOM | 4244 | C | THR | B | 389 | 8.523 | −11.096 | 117.970 | 1.00 | 61.30 | C |
| ATOM | 4245 | O | THR | B | 389 | 8.168 | −9.954 | 118.285 | 1.00 | 61.22 | O |
| ATOM | 4246 | CB | THR | B | 389 | 8.021 | −13.067 | 119.484 | 1.00 | 61.69 | C |
| ATOM | 4247 | OG1 | THR | B | 389 | 6.980 | −12.380 | 120.192 | 1.00 | 61.65 | O |
| ATOM | 4248 | CG2 | THR | B | 389 | 8.615 | −14.162 | 120.377 | 1.00 | 61.60 | C |
| ATOM | 4249 | N | TRP | B | 390 | 8.417 | −11.567 | 116.728 | 1.00 | 60.89 | N |
| ATOM | 4250 | CA | TRP | B | 390 | 7.989 | −10.716 | 115.617 | 1.00 | 60.51 | C |
| ATOM | 4251 | C | TRP | B | 390 | 6.728 | −11.226 | 114.912 | 1.00 | 60.15 | C |
| ATOM | 4252 | O | TRP | B | 390 | 6.528 | −12.435 | 114.774 | 1.00 | 60.17 | O |
| ATOM | 4253 | CB | TRP | B | 390 | 9.131 | −10.539 | 114.605 | 1.00 | 60.56 | C |
| ATOM | 4254 | CG | TRP | B | 390 | 10.385 | −9.913 | 115.183 | 1.00 | 60.76 | C |
| ATOM | 4255 | CD1 | TRP | B | 390 | 11.354 | −10.545 | 115.913 | 1.00 | 60.87 | C |
| ATOM | 4256 | CD2 | TRP | B | 390 | 10.804 | −8.543 | 115.065 | 1.00 | 60.96 | C |
| ATOM | 4257 | NE1 | TRP | B | 390 | 12.343 | −9.656 | 116.261 | 1.00 | 60.96 | N |
| ATOM | 4258 | CE2 | TRP | B | 390 | 12.034 | −8.421 | 115.754 | 1.00 | 60.90 | C |
| ATOM | 4259 | CE3 | TRP | B | 390 | 10.262 | −7.407 | 114.442 | 1.00 | 61.10 | C |
| ATOM | 4260 | CZ2 | TRP | B | 390 | 12.732 | −7.207 | 115.841 | 1.00 | 60.71 | C |
| ATOM | 4261 | CZ3 | TRP | B | 390 | 10.957 | −6.197 | 114.531 | 1.00 | 60.75 | C |
| ATOM | 4262 | CH2 | TRP | B | 390 | 12.178 | −6.111 | 115.227 | 1.00 | 60.81 | C |
| ATOM | 4263 | N | GLY | B | 391 | 5.889 | −10.293 | 114.463 | 1.00 | 59.61 | N |
| ATOM | 4264 | CA | GLY | B | 391 | 4.686 | −10.624 | 113.700 | 1.00 | 59.05 | C |
| ATOM | 4265 | C | GLY | B | 391 | 5.012 | −11.118 | 112.303 | 1.00 | 58.74 | C |
| ATOM | 4266 | O | GLY | B | 391 | 6.179 | −11.213 | 111.926 | 1.00 | 58.88 | O |
| ATOM | 4267 | N | ASP | B | 392 | 3.979 | −11.421 | 111.524 | 1.00 | 58.31 | N |
| ATOM | 4268 | CA | ASP | B | 392 | 4.163 | −11.994 | 110.189 | 1.00 | 57.92 | C |
| ATOM | 4269 | C | ASP | B | 392 | 4.589 | −10.968 | 109.134 | 1.00 | 57.59 | C |
| ATOM | 4270 | O | ASP | B | 392 | 4.887 | −9.817 | 109.458 | 1.00 | 57.63 | O |
| ATOM | 4271 | CB | ASP | B | 392 | 2.895 | −12.735 | 109.747 | 1.00 | 57.96 | C |
| ATOM | 4272 | CG | ASP | B | 392 | 2.647 | −14.000 | 110.549 | 1.00 | 57.76 | C |
| ATOM | 4273 | OD1 | ASP | B | 392 | 3.622 | −14.708 | 110.886 | 1.00 | 57.35 | O |
| ATOM | 4274 | OD2 | ASP | B | 392 | 1.470 | −14.288 | 110.836 | 1.00 | 57.88 | O |
| ATOM | 4275 | N | ARG | B | 393 | 4.621 | −11.400 | 107.874 | 1.00 | 57.12 | N |
| ATOM | 4276 | CA | ARG | B | 393 | 5.016 | −10.538 | 106.762 | 1.00 | 56.62 | C |
| ATOM | 4277 | C | ARG | B | 393 | 3.837 | −9.740 | 106.223 | 1.00 | 55.94 | C |
| ATOM | 4278 | O | ARG | B | 393 | 2.779 | −10.301 | 105.907 | 1.00 | 55.95 | O |
| ATOM | 4279 | CB | ARG | B | 393 | 5.626 | −11.357 | 105.619 | 1.00 | 56.69 | C |
| ATOM | 4280 | CG | ARG | B | 393 | 6.905 | −12.115 | 105.960 | 1.00 | 57.12 | C |
| ATOM | 4281 | CD | ARG | B | 393 | 7.416 | −12.880 | 104.739 | 1.00 | 57.27 | C |
| ATOM | 4282 | NE | ARG | B | 393 | 6.453 | −13.874 | 104.257 | 1.00 | 58.49 | N |
| ATOM | 4283 | CZ | ARG | B | 393 | 6.726 | −15.160 | 104.048 | 1.00 | 58.97 | C |
| ATOM | 4284 | NH1 | ARG | B | 393 | 7.949 | −15.637 | 104.256 | 1.00 | 59.42 | N |
| ATOM | 4285 | NH2 | ARG | B | 393 | 5.773 | −15.973 | 103.612 | 1.00 | 59.05 | N |
| ATOM | 4286 | N | VAL | B | 394 | 4.028 | −8.429 | 106.121 | 1.00 | 55.02 | N |
| ATOM | 4287 | CA | VAL | B | 394 | 3.068 | −7.555 | 105.459 | 1.00 | 54.11 | C |
| ATOM | 4288 | C | VAL | B | 394 | 3.744 | −6.927 | 104.245 | 1.00 | 53.41 | C |
| ATOM | 4289 | O | VAL | B | 394 | 4.675 | −6.126 | 104.381 | 1.00 | 53.48 | O |
| ATOM | 4290 | CB | VAL | B | 394 | 2.540 | −6.454 | 106.403 | 1.00 | 54.16 | C |
| ATOM | 4291 | CG1 | VAL | B | 394 | 1.554 | −5.543 | 105.672 | 1.00 | 54.33 | C |
| ATOM | 4292 | CG2 | VAL | B | 394 | 1.890 | −7.071 | 107.631 | 1.00 | 54.23 | C |
| ATOM | 4293 | N | THR | B | 395 | 3.286 | −7.309 | 103.059 | 1.00 | 52.35 | N |
| ATOM | 4294 | CA | THR | B | 395 | 3.852 | −6.785 | 101.825 | 1.00 | 51.30 | C |
| ATOM | 4295 | C | THR | B | 395 | 3.377 | −5.348 | 101.592 | 1.00 | 50.60 | C |
| ATOM | 4296 | O | THR | B | 395 | 2.174 | −5.077 | 101.528 | 1.00 | 50.44 | O |
| ATOM | 4297 | CB | THR | B | 395 | 3.513 | −7.685 | 100.615 | 1.00 | 51.36 | C |
| ATOM | 4298 | OG1 | THR | B | 395 | 3.799 | −9.051 | 100.938 | 1.00 | 51.10 | O |
| ATOM | 4299 | CG2 | THR | B | 395 | 4.328 | −7.279 | 99.392 | 1.00 | 51.41 | C |
| ATOM | 4300 | N | ILE | B | 396 | 4.339 | −4.435 | 101.498 | 1.00 | 49.63 | N |
| ATOM | 4301 | CA | ILE | B | 396 | 4.070 | −3.032 | 101.201 | 1.00 | 48.62 | C |
| ATOM | 4302 | C | ILE | B | 396 | 3.750 | −2.879 | 99.715 | 1.00 | 47.87 | C |
| ATOM | 4303 | O | ILE | B | 396 | 2.710 | −2.329 | 99.348 | 1.00 | 47.76 | O |
| ATOM | 4304 | CB | ILE | B | 396 | 5.269 | −2.128 | 101.593 | 1.00 | 48.69 | C |
| ATOM | 4305 | CG1 | ILE | B | 396 | 5.470 | −2.123 | 103.115 | 1.00 | 48.69 | C |
| ATOM | 4306 | CG2 | ILE | B | 396 | 5.075 | −0.710 | 101.060 | 1.00 | 48.75 | C |
| ATOM | 4307 | CD1 | ILE | B | 396 | 6.683 | −1.323 | 103.598 | 1.00 | 48.63 | C |
| ATOM | 4308 | N | THR | B | 397 | 4.654 | −3.370 | 98.872 | 1.00 | 47.01 | N |
| ATOM | 4309 | CA | THR | B | 397 | 4.486 | −3.347 | 97.423 | 1.00 | 46.16 | C |
| ATOM | 4310 | C | THR | B | 397 | 5.247 | −4.513 | 96.810 | 1.00 | 45.64 | C |
| ATOM | 4311 | O | THR | B | 397 | 6.390 | −4.787 | 97.188 | 1.00 | 45.47 | O |
| ATOM | 4312 | CB | THR | B | 397 | 5.021 | −2.027 | 96.794 | 1.00 | 46.19 | C |
| ATOM | 4313 | OG1 | THR | B | 397 | 4.451 | −0.895 | 97.461 | 1.00 | 46.02 | O |
| ATOM | 4314 | CG2 | THR | B | 397 | 4.683 | −1.952 | 95.310 | 1.00 | 45.89 | C |
| ATOM | 4315 | N | ASN | B | 398 | 4.601 | −5.208 | 95.881 | 1.00 | 44.97 | N |
| ATOM | 4316 | CA | ASN | B | 398 | 5.290 | −6.151 | 95.013 | 1.00 | 44.52 | C |
| ATOM | 4317 | C | ASN | B | 398 | 4.607 | −6.196 | 93.667 | 1.00 | 44.22 | C |
| ATOM | 4318 | O | ASN | B | 398 | 3.491 | −6.702 | 93.547 | 1.00 | 44.41 | O |
| ATOM | 4319 | CB | ASN | B | 398 | 5.347 | −7.553 | 95.624 | 1.00 | 44.55 | C |
| ATOM | 4320 | CG | ASN | B | 398 | 6.351 | −8.459 | 94.921 | 1.00 | 44.54 | C |
| ATOM | 4321 | OD1 | ASN | B | 398 | 6.687 | −8.267 | 93.746 | 1.00 | 44.31 | O |
| ATOM | 4322 | ND2 | ASN | B | 398 | 6.839 | −9.456 | 95.648 | 1.00 | 44.71 | N |
| ATOM | 4323 | N | LEU | B | 399 | 5.279 | −5.659 | 92.656 | 1.00 | 43.79 | N |
| ATOM | 4324 | CA | LEU | B | 399 | 4.722 | −5.620 | 91.311 | 1.00 | 43.45 | C |
| ATOM | 4325 | C | LEU | B | 399 | 4.800 | −7.002 | 90.681 | 1.00 | 43.31 | C |
| ATOM | 4326 | O | LEU | B | 399 | 5.805 | −7.697 | 90.829 | 1.00 | 43.25 | O |
| ATOM | 4327 | CB | LEU | B | 399 | 5.442 | −4.577 | 90.451 | 1.00 | 43.32 | C |
| ATOM | 4328 | CG | LEU | B | 399 | 5.396 | −3.125 | 90.948 | 1.00 | 43.02 | C |
| ATOM | 4329 | CD1 | LEU | B | 399 | 6.385 | −2.259 | 90.187 | 1.00 | 42.65 | C |
| ATOM | 4330 | CD2 | LEU | B | 399 | 3.994 | −2.542 | 90.863 | 1.00 | 42.52 | C |
| ATOM | 4331 | N | ARG | B | 400 | 3.727 | −7.402 | 90.002 | 1.00 | 43.23 | N |
| ATOM | 4332 | CA | ARG | B | 400 | 3.667 | −8.713 | 89.365 | 1.00 | 43.33 | C |
| ATOM | 4333 | C | ARG | B | 400 | 4.728 | −8.845 | 88.273 | 1.00 | 43.68 | C |
| ATOM | 4334 | O | ARG | B | 400 | 5.140 | −7.849 | 87.670 | 1.00 | 43.71 | O |
| ATOM | 4335 | CB | ARG | B | 400 | 2.264 | −9.007 | 88.819 | 1.00 | 43.24 | C |
| ATOM | 4336 | CG | ARG | B | 400 | 1.805 | −8.133 | 87.651 | 1.00 | 43.22 | C |
| ATOM | 4337 | CD | ARG | B | 400 | 0.375 | −8.483 | 87.256 | 1.00 | 43.00 | C |
| ATOM | 4338 | NE | ARG | B | 400 | −0.127 | −7.677 | 86.144 | 1.00 | 42.29 | N |
| ATOM | 4339 | CZ | ARG | B | 400 | −0.216 | −8.095 | 84.883 | 1.00 | 42.43 | C |
| ATOM | 4340 | NH1 | ARG | B | 400 | 0.172 | −9.320 | 84.547 | 1.00 | 42.64 | N |
| ATOM | 4341 | NH2 | ARG | B | 400 | −0.692 | −7.284 | 83.949 | 1.00 | 42.17 | N |
| ATOM | 4342 | N | ASP | B | 401 | 5.179 | −10.074 | 88.041 | 1.00 | 43.91 | N |
| ATOM | 4343 | CA | ASP | B | 401 | 6.196 | −10.344 | 87.032 | 1.00 | 44.05 | C |
| ATOM | 4344 | C | ASP | B | 401 | 5.606 | −10.260 | 85.625 | 1.00 | 43.88 | C |
| ATOM | 4345 | O | ASP | B | 401 | 4.388 | −10.177 | 85.458 | 1.00 | 43.93 | O |
| ATOM | 4346 | CB | ASP | B | 401 | 6.843 | −11.717 | 87.269 | 1.00 | 44.27 | C |
| ATOM | 4347 | CG | ASP | B | 401 | 5.852 | −12.873 | 87.130 | 1.00 | 45.42 | C |
| ATOM | 4348 | OD1 | ASP | B | 401 | 4.777 | −12.830 | 87.775 | 1.00 | 46.37 | O |
| ATOM | 4349 | OD2 | ASP | B | 401 | 6.159 | −13.835 | 86.385 | 1.00 | 46.40 | O |
| ATOM | 4350 | N | ASN | B | 402 | 6.481 | −10.243 | 84.624 | 1.00 | 43.70 | N |
| ATOM | 4351 | CA | ASN | B | 402 | 6.067 | −10.419 | 83.243 | 1.00 | 43.53 | C |
| ATOM | 4352 | C | ASN | B | 402 | 6.002 | −11.918 | 82.994 | 1.00 | 43.56 | C |
| ATOM | 4353 | O | ASN | B | 402 | 7.033 | −12.594 | 83.033 | 1.00 | 43.58 | O |
| ATOM | 4354 | CB | ASN | B | 402 | 7.058 | −9.750 | 82.283 | 1.00 | 43.44 | C |
| ATOM | 4355 | CG | ASN | B | 402 | 6.608 | −9.803 | 80.827 | 1.00 | 43.21 | C |
| ATOM | 4356 | OD1 | ASN | B | 402 | 5.542 | −10.331 | 80.506 | 1.00 | 43.76 | O |
| ATOM | 4357 | ND2 | ASN | B | 402 | 7.424 | −9.248 | 79.940 | 1.00 | 42.61 | N |
| ATOM | 4358 | N | PRO | B | 403 | 4.787 | −12.451 | 82.767 | 1.00 | 43.51 | N |
| ATOM | 4359 | CA | PRO | B | 403 | 4.658 | −13.883 | 82.517 | 1.00 | 43.41 | C |
| ATOM | 4360 | C | PRO | B | 403 | 5.164 | −14.273 | 81.128 | 1.00 | 43.36 | C |
| ATOM | 4361 | O | PRO | B | 403 | 5.332 | −15.459 | 80.851 | 1.00 | 43.35 | O |
| ATOM | 4362 | CB | PRO | B | 403 | 3.148 | −14.119 | 82.626 | 1.00 | 43.42 | C |
| ATOM | 4363 | CG | PRO | B | 403 | 2.538 | −12.818 | 82.239 | 1.00 | 43.40 | C |
| ATOM | 4364 | CD | PRO | B | 403 | 3.482 | −11.761 | 82.737 | 1.00 | 43.45 | C |
| ATOM | 4365 | N | LYS | B | 404 | 5.400 | −13.281 | 80.270 | 1.00 | 43.25 | N |
| ATOM | 4366 | CA | LYS | B | 404 | 5.856 | −13.530 | 78.898 | 1.00 | 43.29 | C |
| ATOM | 4367 | C | LYS | B | 404 | 7.365 | −13.346 | 78.695 | 1.00 | 43.18 | C |
| ATOM | 4368 | O | LYS | B | 404 | 7.899 | −13.706 | 77.645 | 1.00 | 43.11 | O |
| ATOM | 4369 | CB | LYS | B | 404 | 5.069 | −12.674 | 77.897 | 1.00 | 43.20 | C |
| ATOM | 4370 | CG | LYS | B | 404 | 3.594 | −13.049 | 77.776 | 1.00 | 43.76 | C |
| ATOM | 4371 | CD | LYS | B | 404 | 3.399 | −14.420 | 77.142 | 1.00 | 44.35 | C |
| ATOM | 4372 | CE | LYS | B | 404 | 1.998 | −14.960 | 77.402 | 1.00 | 44.96 | C |
| ATOM | 4373 | NZ | LYS | B | 404 | 0.967 | −14.304 | 76.546 | 1.00 | 45.38 | N |
| ATOM | 4374 | N | ALA | B | 405 | 8.039 | −12.788 | 79.701 | 1.00 | 43.05 | N |
| ATOM | 4375 | CA | ALA | B | 405 | 9.483 | −12.562 | 79.655 | 1.00 | 42.95 | C |
| ATOM | 4376 | C | ALA | B | 405 | 10.259 | −13.872 | 79.541 | 1.00 | 42.96 | C |
| ATOM | 4377 | O | ALA | B | 405 | 10.077 | −14.790 | 80.351 | 1.00 | 43.00 | O |
| ATOM | 4378 | CB | ALA | B | 405 | 9.936 | −11.782 | 80.881 | 1.00 | 42.92 | C |
| ATOM | 4379 | N | SER | B | 406 | 11.133 | −13.942 | 78.539 | 1.00 | 42.86 | N |
| ATOM | 4380 | CA | SER | B | 406 | 11.881 | −15.164 | 78.233 | 1.00 | 42.69 | C |
| ATOM | 4381 | C | SER | B | 406 | 12.944 | −15.521 | 79.283 | 1.00 | 42.54 | C |
| ATOM | 4382 | O | SER | B | 406 | 13.562 | −16.584 | 79.205 | 1.00 | 42.55 | O |
| ATOM | 4383 | CB | SER | B | 406 | 12.502 | −15.072 | 76.836 | 1.00 | 42.73 | C |
| ATOM | 4384 | OG | SER | B | 406 | 13.423 | −14.001 | 76.763 | 1.00 | 42.63 | O |
| ATOM | 4385 | N | ASP | B | 407 | 13.140 | −14.638 | 80.263 | 1.00 | 42.43 | N |
| ATOM | 4386 | CA | ASP | B | 407 | 14.057 | −14.881 | 81.383 | 1.00 | 42.09 | C |
| ATOM | 4387 | C | ASP | B | 407 | 13.456 | −14.316 | 82.671 | 1.00 | 42.12 | C |
| ATOM | 4388 | O | ASP | B | 407 | 13.482 | −13.101 | 82.884 | 1.00 | 42.19 | O |
| ATOM | 4389 | CB | ASP | B | 407 | 15.432 | −14.254 | 81.103 | 1.00 | 41.88 | C |
| ATOM | 4390 | CG | ASP | B | 407 | 16.457 | −14.531 | 82.205 | 1.00 | 41.41 | C |
| ATOM | 4391 | OD1 | ASP | B | 407 | 16.109 | −15.118 | 83.253 | 1.00 | 40.91 | O |
| ATOM | 4392 | OD2 | ASP | B | 407 | 17.632 | −14.154 | 82.013 | 1.00 | 40.70 | O |
| ATOM | 4393 | N | PRO | B | 408 | 12.909 | −15.196 | 83.534 | 1.00 | 42.01 | N |
| ATOM | 4394 | CA | PRO | B | 408 | 12.267 | −14.760 | 84.779 | 1.00 | 41.78 | C |
| ATOM | 4395 | C | PRO | B | 408 | 13.180 | −13.985 | 85.740 | 1.00 | 41.65 | C |
| ATOM | 4396 | O | PRO | B | 408 | 12.686 | −13.145 | 86.497 | 1.00 | 41.65 | O |
| ATOM | 4397 | CB | PRO | B | 408 | 11.807 | −16.077 | 85.417 | 1.00 | 41.81 | C |
| ATOM | 4398 | CG | PRO | B | 408 | 11.694 | −17.034 | 84.270 | 1.00 | 41.90 | C |
| ATOM | 4399 | CD | PRO | B | 408 | 12.839 | −16.660 | 83.375 | 1.00 | 42.12 | C |
| ATOM | 4400 | N | SER | B | 409 | 14.486 | −14.254 | 85.704 | 1.00 | 41.25 | N |
| ATOM | 4401 | CA | SER | B | 409 | 15.439 | −13.614 | 86.621 | 1.00 | 40.98 | C |
| ATOM | 4402 | C | SER | B | 409 | 15.524 | −12.099 | 86.433 | 1.00 | 40.65 | C |
| ATOM | 4403 | O | SER | B | 409 | 15.719 | −11.360 | 87.398 | 1.00 | 40.63 | O |
| ATOM | 4404 | CB | SER | B | 409 | 16.832 | −14.240 | 86.494 | 1.00 | 41.07 | C |
| ATOM | 4405 | OG | SER | B | 409 | 17.441 | −13.892 | 85.261 | 1.00 | 41.54 | O |
| ATOM | 4406 | N | ILE | B | 410 | 15.382 | −11.651 | 85.189 | 1.00 | 40.26 | N |
| ATOM | 4407 | CA | ILE | B | 410 | 15.348 | −10.223 | 84.867 | 1.00 | 39.83 | C |
| ATOM | 4408 | C | ILE | B | 410 | 13.993 | −9.813 | 84.277 | 1.00 | 39.58 | C |
| ATOM | 4409 | O | ILE | B | 410 | 13.875 | −8.776 | 83.626 | 1.00 | 39.49 | O |
| ATOM | 4410 | CB | ILE | B | 410 | 16.492 | −9.826 | 83.889 | 1.00 | 40.07 | C |
| ATOM | 4411 | CG1 | ILE | B | 410 | 16.392 | −10.604 | 82.565 | 1.00 | 39.81 | C |
| ATOM | 4412 | CG2 | ILE | B | 410 | 17.859 | −10.019 | 84.551 | 1.00 | 39.82 | C |
| ATOM | 4413 | CD1 | ILE | B | 410 | 17.123 | −9.948 | 81.396 | 1.00 | 39.65 | C |
| ATOM | 4414 | N | GLY | B | 411 | 12.972 | −10.628 | 84.522 | 1.00 | 39.32 | N |
| ATOM | 4415 | CA | GLY | B | 411 | 11.692 | −10.475 | 83.838 | 1.00 | 39.13 | C |
| ATOM | 4416 | C | GLY | B | 411 | 10.577 | −9.865 | 84.659 | 1.00 | 38.89 | C |
| ATOM | 4417 | O | GLY | B | 411 | 9.408 | −10.222 | 84.488 | 1.00 | 38.80 | O |
| ATOM | 4418 | N | SER | B | 412 | 10.934 | −8.934 | 85.539 | 1.00 | 38.61 | N |
| ATOM | 4419 | CA | SER | B | 412 | 9.958 | −8.315 | 86.425 | 1.00 | 38.45 | C |
| ATOM | 4420 | C | SER | B | 412 | 10.272 | −6.847 | 86.663 | 1.00 | 38.34 | C |
| ATOM | 4421 | O | SER | B | 412 | 11.442 | −6.474 | 86.737 | 1.00 | 38.44 | O |
| ATOM | 4422 | CB | SER | B | 412 | 9.906 | −9.062 | 87.762 | 1.00 | 38.54 | C |
| ATOM | 4423 | OG | SER | B | 412 | 9.226 | −8.312 | 88.752 | 1.00 | 38.11 | O |
| ATOM | 4424 | N | PRO | B | 413 | 9.224 | −6.006 | 86.766 | 1.00 | 38.25 | N |
| ATOM | 4425 | CA | PRO | B | 413 | 9.381 | −4.645 | 87.269 | 1.00 | 38.04 | C |
| ATOM | 4426 | C | PRO | B | 413 | 9.919 | −4.707 | 88.689 | 1.00 | 38.02 | C |
| ATOM | 4427 | O | PRO | B | 413 | 9.565 | −5.616 | 89.441 | 1.00 | 38.02 | O |
| ATOM | 4428 | CB | PRO | B | 413 | 7.952 | −4.100 | 87.267 | 1.00 | 38.05 | C |
| ATOM | 4429 | CG | PRO | B | 413 | 7.217 | −4.945 | 86.295 | 1.00 | 38.13 | C |
| ATOM | 4430 | CD | PRO | B | 413 | 7.829 | −6.296 | 86.387 | 1.00 | 38.17 | C |
| ATOM | 4431 | N | VAL | B | 414 | 10.756 | −3.739 | 89.047 | 1.00 | 38.00 | N |
| ATOM | 4432 | CA | VAL | B | 414 | 11.584 | −3.829 | 90.241 | 1.00 | 38.01 | C |
| ATOM | 4433 | C | VAL | B | 414 | 11.307 | −2.723 | 91.265 | 1.00 | 38.24 | C |
| ATOM | 4434 | O | VAL | B | 414 | 11.176 | −1.548 | 90.916 | 1.00 | 38.31 | O |
| ATOM | 4435 | CB | VAL | B | 414 | 13.100 | −3.816 | 89.854 | 1.00 | 37.97 | C |
| ATOM | 4436 | CG1 | VAL | B | 414 | 14.000 | −3.888 | 91.087 | 1.00 | 38.00 | C |
| ATOM | 4437 | CG2 | VAL | B | 414 | 13.429 | −4.959 | 88.896 | 1.00 | 37.76 | C |
| ATOM | 4438 | N | ASN | B | 415 | 11.204 | −3.121 | 92.531 | 1.00 | 38.37 | N |
| ATOM | 4439 | CA | ASN | B | 415 | 11.379 | −2.204 | 93.650 | 1.00 | 38.54 | C |
| ATOM | 4440 | C | ASN | B | 415 | 12.604 | −2.646 | 94.428 | 1.00 | 38.73 | C |
| ATOM | 4441 | O | ASN | B | 415 | 12.893 | −3.839 | 94.499 | 1.00 | 38.71 | O |
| ATOM | 4442 | CB | ASN | B | 415 | 10.143 | −2.170 | 94.540 | 1.00 | 38.44 | C |
| ATOM | 4443 | CG | ASN | B | 415 | 8.976 | −1.478 | 93.879 | 1.00 | 38.66 | C |
| ATOM | 4444 | OD1 | ASN | B | 415 | 9.075 | −0.319 | 93.468 | 1.00 | 38.26 | O |
| ATOM | 4445 | ND2 | ASN | B | 415 | 7.859 | −2.186 | 93.765 | 1.00 | 39.63 | N |
| ATOM | 4446 | N | ILE | B | 416 | 13.332 | −1.691 | 94.999 | 1.00 | 39.03 | N |
| ATOM | 4447 | CA | ILE | B | 416 | 14.634 | −1.985 | 95.601 | 1.00 | 39.28 | C |
| ATOM | 4448 | C | ILE | B | 416 | 15.098 | −0.855 | 96.521 | 1.00 | 39.52 | C |
| ATOM | 4449 | O | ILE | B | 416 | 14.970 | 0.316 | 96.171 | 1.00 | 39.66 | O |
| ATOM | 4450 | CB | ILE | B | 416 | 15.690 | −2.306 | 94.495 | 1.00 | 39.29 | C |
| ATOM | 4451 | CG1 | ILE | B | 416 | 17.053 | −2.656 | 95.096 | 1.00 | 39.12 | C |
| ATOM | 4452 | CG2 | ILE | B | 416 | 15.783 | −1.174 | 93.458 | 1.00 | 39.35 | C |
| ATOM | 4453 | CD1 | ILE | B | 416 | 17.960 | −3.371 | 94.122 | 1.00 | 38.77 | C |
| ATOM | 4454 | N | ASP | B | 417 | 15.620 | −1.221 | 97.696 | 1.00 | 39.86 | N |
| ATOM | 4455 | CA | ASP | B | 417 | 16.148 | −0.273 | 98.703 | 1.00 | 40.28 | C |
| ATOM | 4456 | C | ASP | B | 417 | 15.056 | 0.593 | 99.325 | 1.00 | 40.68 | C |
| ATOM | 4457 | O | ASP | B | 417 | 14.347 | 1.301 | 98.616 | 1.00 | 40.89 | O |
| ATOM | 4458 | CB | ASP | B | 417 | 17.246 | 0.642 | 98.126 | 1.00 | 39.98 | C |
| ATOM | 4459 | CG | ASP | B | 417 | 18.463 | −0.121 | 97.620 | 1.00 | 39.77 | C |
| ATOM | 4460 | OD1 | ASP | B | 417 | 18.707 | −1.270 | 98.055 | 1.00 | 39.27 | O |
| ATOM | 4461 | OD2 | ASP | B | 417 | 19.192 | 0.450 | 96.782 | 1.00 | 39.23 | O |
| ATOM | 4462 | N | MET | B | 418 | 14.930 | 0.557 | 100.647 | 1.00 | 41.03 | N |
| ATOM | 4463 | CA | MET | B | 418 | 13.948 | 1.406 | 101.315 | 1.00 | 41.62 | C |
| ATOM | 4464 | C | MET | B | 418 | 14.532 | 2.235 | 102.456 | 1.00 | 41.60 | C |
| ATOM | 4465 | O | MET | B | 418 | 15.510 | 1.841 | 103.101 | 1.00 | 41.31 | O |
| ATOM | 4466 | CB | MET | B | 418 | 12.749 | 0.585 | 101.804 | 1.00 | 41.57 | C |
| ATOM | 4467 | CG | MET | B | 418 | 13.014 | −0.253 | 103.051 | 1.00 | 42.12 | C |
| ATOM | 4468 | SD | MET | B | 418 | 11.604 | −1.265 | 103.541 | 1.00 | 42.95 | S |
| ATOM | 4469 | CE | MET | B | 418 | 10.501 | −0.042 | 104.219 | 1.00 | 42.03 | C |
| ATOM | 4470 | N | VAL | B | 419 | 13.923 | 3.398 | 102.672 | 1.00 | 41.88 | N |
| ATOM | 4471 | CA | VAL | B | 419 | 14.166 | 4.214 | 103.855 | 1.00 | 42.05 | C |
| ATOM | 4472 | C | VAL | B | 419 | 12.874 | 4.245 | 104.669 | 1.00 | 42.21 | C |
| ATOM | 4473 | O | VAL | B | 419 | 11.789 | 4.432 | 104.113 | 1.00 | 41.92 | O |
| ATOM | 4474 | CB | VAL | B | 419 | 14.605 | 5.658 | 103.488 | 1.00 | 42.04 | C |
| ATOM | 4475 | CG1 | VAL | B | 419 | 14.719 | 6.534 | 104.731 | 1.00 | 42.00 | C |
| ATOM | 4476 | CG2 | VAL | B | 419 | 15.925 | 5.648 | 102.746 | 1.00 | 42.11 | C |
| ATOM | 4477 | N | LEU | B | 420 | 12.994 | 4.038 | 105.979 | 1.00 | 42.57 | N |
| ATOM | 4478 | CA | LEU | B | 420 | 11.866 | 4.209 | 106.893 | 1.00 | 42.93 | C |
| ATOM | 4479 | C | LEU | B | 420 | 12.037 | 5.482 | 107.707 | 1.00 | 43.21 | C |
| ATOM | 4480 | O | LEU | B | 420 | 13.161 | 5.883 | 108.000 | 1.00 | 43.35 | O |
| ATOM | 4481 | CB | LEU | B | 420 | 11.729 | 3.002 | 107.823 | 1.00 | 42.97 | C |
| ATOM | 4482 | CG | LEU | B | 420 | 11.101 | 1.734 | 107.246 | 1.00 | 42.73 | C |
| ATOM | 4483 | CD1 | LEU | B | 420 | 11.460 | 0.525 | 108.092 | 1.00 | 42.83 | C |
| ATOM | 4484 | CD2 | LEU | B | 420 | 9.585 | 1.874 | 107.124 | 1.00 | 43.15 | C |
| ATOM | 4485 | N | VAL | B | 421 | 10.923 | 6.118 | 108.057 | 1.00 | 43.75 | N |
| ATOM | 4486 | CA | VAL | B | 421 | 10.935 | 7.322 | 108.898 | 1.00 | 44.40 | C |
| ATOM | 4487 | C | VAL | B | 421 | 9.526 | 7.629 | 109.425 | 1.00 | 44.99 | C |
| ATOM | 4488 | O | VAL | B | 421 | 8.532 | 7.289 | 108.776 | 1.00 | 45.16 | O |
| ATOM | 4489 | CB | VAL | B | 421 | 11.540 | 8.549 | 108.140 | 1.00 | 44.28 | C |
| ATOM | 4490 | CG1 | VAL | B | 421 | 10.610 | 9.035 | 107.028 | 1.00 | 44.12 | C |
| ATOM | 4491 | CG2 | VAL | B | 421 | 11.903 | 9.678 | 109.105 | 1.00 | 44.04 | C |
| ATOM | 4492 | N | GLN | B | 422 | 9.442 | 8.260 | 110.598 | 1.00 | 45.55 | N |
| ATOM | 4493 | CA | GLN | B | 422 | 8.150 | 8.670 | 111.151 | 1.00 | 46.17 | C |
| ATOM | 4494 | C | GLN | B | 422 | 8.069 | 10.164 | 111.475 | 1.00 | 46.61 | C |
| ATOM | 4495 | O | GLN | B | 422 | 8.987 | 10.731 | 112.075 | 1.00 | 46.70 | O |
| ATOM | 4496 | CB | GLN | B | 422 | 7.795 | 7.851 | 112.394 | 1.00 | 46.05 | C |
| ATOM | 4497 | CG | GLN | B | 422 | 6.360 | 8.076 | 112.866 | 1.00 | 46.24 | C |
| ATOM | 4498 | CD | GLN | B | 422 | 6.026 | 7.353 | 114.152 | 1.00 | 46.24 | C |
| ATOM | 4499 | OE1 | GLN | B | 422 | 6.860 | 7.225 | 115.052 | 1.00 | 45.64 | O |
| ATOM | 4500 | NE2 | GLN | B | 422 | 4.789 | 6.882 | 114.249 | 1.00 | 46.60 | N |
| ATOM | 4501 | N | ASP | B | 423 | 6.960 | 10.786 | 111.074 | 1.00 | 47.04 | N |
| ATOM | 4502 | CA | ASP | B | 423 | 6.656 | 12.161 | 111.462 | 1.00 | 47.59 | C |
| ATOM | 4503 | C | ASP | B | 423 | 6.200 | 12.179 | 112.923 | 1.00 | 47.71 | C |
| ATOM | 4504 | O | ASP | B | 423 | 5.211 | 11.530 | 113.265 | 1.00 | 47.74 | O |
| ATOM | 4505 | CB | ASP | B | 423 | 5.575 | 12.756 | 110.549 | 1.00 | 47.68 | C |
| ATOM | 4506 | CG | ASP | B | 423 | 5.374 | 14.250 | 110.769 | 1.00 | 48.46 | C |
| ATOM | 4507 | OD1 | ASP | B | 423 | 5.604 | 15.027 | 109.815 | 1.00 | 48.91 | O |
| ATOM | 4508 | OD2 | ASP | B | 423 | 4.994 | 14.654 | 111.892 | 1.00 | 49.14 | O |
| ATOM | 4509 | N | PRO | B | 424 | 6.921 | 12.922 | 113.788 | 1.00 | 47.92 | N |
| ATOM | 4510 | CA | PRO | B | 424 | 6.616 | 12.940 | 115.227 | 1.00 | 47.97 | C |
| ATOM | 4511 | C | PRO | B | 424 | 5.249 | 13.547 | 115.574 | 1.00 | 47.86 | C |
| ATOM | 4512 | O | PRO | B | 424 | 4.643 | 13.157 | 116.574 | 1.00 | 48.17 | O |
| ATOM | 4513 | CB | PRO | B | 424 | 7.753 | 13.784 | 115.824 | 1.00 | 48.04 | C |
| ATOM | 4514 | CG | PRO | B | 424 | 8.254 | 14.619 | 114.694 | 1.00 | 48.06 | C |
| ATOM | 4515 | CD | PRO | B | 424 | 8.073 | 13.783 | 113.458 | 1.00 | 48.02 | C |
| ATOM | 4516 | N | GLU | B | 425 | 4.770 | 14.475 | 114.750 | 1.00 | 47.46 | N |
| ATOM | 4517 | CA | GLU | B | 425 | 3.507 | 15.159 | 115.007 | 1.00 | 47.16 | C |
| ATOM | 4518 | C | GLU | B | 425 | 2.303 | 14.416 | 114.429 | 1.00 | 46.87 | C |
| ATOM | 4519 | O | GLU | B | 425 | 1.236 | 14.394 | 115.042 | 1.00 | 46.97 | O |
| ATOM | 4520 | CB | GLU | B | 425 | 3.556 | 16.598 | 114.482 | 1.00 | 47.12 | C |
| ATOM | 4521 | CG | GLU | B | 425 | 4.616 | 17.468 | 115.160 | 1.00 | 47.30 | C |
| ATOM | 4522 | CD | GLU | B | 425 | 4.710 | 18.877 | 114.584 | 1.00 | 47.67 | C |
| ATOM | 4523 | OE1 | GLU | B | 425 | 4.028 | 19.176 | 113.576 | 1.00 | 47.76 | O |
| ATOM | 4524 | OE2 | GLU | B | 425 | 5.476 | 19.692 | 115.148 | 1.00 | 48.52 | O |
| ATOM | 4525 | N | THR | B | 426 | 2.480 | 13.805 | 113.260 | 1.00 | 46.52 | N |
| ATOM | 4526 | CA | THR | B | 426 | 1.393 | 13.105 | 112.561 | 1.00 | 46.07 | C |
| ATOM | 4527 | C | THR | B | 426 | 1.399 | 11.595 | 112.822 | 1.00 | 45.59 | C |
| ATOM | 4528 | O | THR | B | 426 | 0.370 | 10.932 | 112.680 | 1.00 | 45.48 | O |
| ATOM | 4529 | CB | THR | B | 426 | 1.450 | 13.372 | 111.036 | 1.00 | 46.07 | C |
| ATOM | 4530 | OG1 | THR | B | 426 | 1.455 | 14.781 | 110.799 | 1.00 | 46.12 | O |
| ATOM | 4531 | CG2 | THR | B | 426 | 0.252 | 12.760 | 110.318 | 1.00 | 46.59 | C |
| ATOM | 4532 | N | LYS | B | 427 | 2.560 | 11.064 | 113.204 | 1.00 | 45.07 | N |
| ATOM | 4533 | CA | LYS | B | 427 | 2.752 | 9.626 | 113.465 | 1.00 | 44.76 | C |
| ATOM | 4534 | C | LYS | B | 427 | 2.705 | 8.766 | 112.197 | 1.00 | 44.21 | C |
| ATOM | 4535 | O | LYS | B | 427 | 2.760 | 7.535 | 112.267 | 1.00 | 44.22 | O |
| ATOM | 4536 | CB | LYS | B | 427 | 1.764 | 9.093 | 114.522 | 1.00 | 44.77 | C |
| ATOM | 4537 | CG | LYS | B | 427 | 1.578 | 9.976 | 115.749 | 1.00 | 45.36 | C |
| ATOM | 4538 | CD | LYS | B | 427 | 2.877 | 10.242 | 116.482 | 1.00 | 46.44 | C |
| ATOM | 4539 | CE | LYS | B | 427 | 2.604 | 10.927 | 117.811 | 1.00 | 47.45 | C |
| ATOM | 4540 | NZ | LYS | B | 427 | 3.866 | 11.357 | 118.470 | 1.00 | 48.00 | N |
| ATOM | 4541 | N | ARG | B | 428 | 2.608 | 9.421 | 111.044 | 1.00 | 43.60 | N |
| ATOM | 4542 | CA | ARG | B | 428 | 2.651 | 8.738 | 109.761 | 1.00 | 42.88 | C |
| ATOM | 4543 | C | ARG | B | 428 | 4.050 | 8.182 | 109.526 | 1.00 | 42.34 | C |
| ATOM | 4544 | O | ARG | B | 428 | 5.051 | 8.883 | 109.697 | 1.00 | 42.25 | O |
| ATOM | 4545 | CB | ARG | B | 428 | 2.239 | 9.686 | 108.630 | 1.00 | 42.98 | C |
| ATOM | 4546 | CG | ARG | B | 428 | 2.139 | 9.026 | 107.259 | 1.00 | 42.73 | C |
| ATOM | 4547 | CD | ARG | B | 428 | 1.397 | 9.904 | 106.269 | 1.00 | 42.52 | C |
| ATOM | 4548 | NE | ARG | B | 428 | −0.052 | 9.784 | 106.413 | 1.00 | 43.12 | N |
| ATOM | 4549 | CZ | ARG | B | 428 | −0.949 | 10.454 | 105.690 | 1.00 | 42.82 | C |
| ATOM | 4550 | NH1 | ARG | B | 428 | −0.562 | 11.312 | 104.752 | 1.00 | 42.35 | N |
| ATOM | 4551 | NH2 | ARG | B | 428 | −2.244 | 10.263 | 105.907 | 1.00 | 42.41 | N |
| ATOM | 4552 | N | ILE | B | 429 | 4.100 | 6.907 | 109.161 | 1.00 | 41.66 | N |
| ATOM | 4553 | CA | ILE | B | 429 | 5.351 | 6.219 | 108.882 | 1.00 | 41.07 | C |
| ATOM | 4554 | C | ILE | B | 429 | 5.529 | 6.139 | 107.369 | 1.00 | 40.60 | C |
| ATOM | 4555 | O | ILE | B | 429 | 4.619 | 5.719 | 106.647 | 1.00 | 40.85 | O |
| ATOM | 4556 | CB | ILE | B | 429 | 5.371 | 4.807 | 109.528 | 1.00 | 41.23 | C |
| ATOM | 4557 | CG1 | ILE | B | 429 | 5.381 | 4.924 | 111.058 | 1.00 | 41.42 | C |
| ATOM | 4558 | CG2 | ILE | B | 429 | 6.577 | 3.994 | 109.054 | 1.00 | 41.11 | C |
| ATOM | 4559 | CD1 | ILE | B | 429 | 4.714 | 3.766 | 111.779 | 1.00 | 41.39 | C |
| ATOM | 4560 | N | PHE | B | 430 | 6.698 | 6.560 | 106.897 | 1.00 | 39.78 | N |
| ATOM | 4561 | CA | PHE | B | 430 | 6.987 | 6.591 | 105.468 | 1.00 | 38.90 | C |
| ATOM | 4562 | C | PHE | B | 430 | 7.968 | 5.508 | 105.040 | 1.00 | 38.31 | C |
| ATOM | 4563 | O | PHE | B | 430 | 8.939 | 5.212 | 105.736 | 1.00 | 38.19 | O |
| ATOM | 4564 | CB | PHE | B | 430 | 7.525 | 7.965 | 105.057 | 1.00 | 39.03 | C |
| ATOM | 4565 | CG | PHE | B | 430 | 6.533 | 9.079 | 105.224 | 1.00 | 38.86 | C |
| ATOM | 4566 | CD1 | PHE | B | 430 | 6.572 | 9.899 | 106.347 | 1.00 | 38.81 | C |
| ATOM | 4567 | CD2 | PHE | B | 430 | 5.552 | 9.304 | 104.263 | 1.00 | 38.79 | C |
| ATOM | 4568 | CE1 | PHE | B | 430 | 5.648 | 10.930 | 106.509 | 1.00 | 39.24 | C |
| ATOM | 4569 | CE2 | PHE | B | 430 | 4.626 | 10.334 | 104.410 | 1.00 | 38.59 | C |
| ATOM | 4570 | CZ | PHE | B | 430 | 4.671 | 11.147 | 105.533 | 1.00 | 39.08 | C |
| ATOM | 4571 | N | SER | B | 431 | 7.690 | 4.920 | 103.884 | 1.00 | 37.66 | N |
| ATOM | 4572 | CA | SER | B | 431 | 8.615 | 4.017 | 103.226 | 1.00 | 36.94 | C |
| ATOM | 4573 | C | SER | B | 431 | 8.901 | 4.574 | 101.834 | 1.00 | 36.48 | C |
| ATOM | 4574 | O | SER | B | 431 | 8.025 | 4.575 | 100.965 | 1.00 | 36.50 | O |
| ATOM | 4575 | CB | SER | B | 431 | 8.037 | 2.602 | 103.142 | 1.00 | 36.87 | C |
| ATOM | 4576 | OG | SER | B | 431 | 8.873 | 1.759 | 102.363 | 1.00 | 36.72 | O |
| ATOM | 4577 | N | ILE | B | 432 | 10.119 | 5.069 | 101.639 | 1.00 | 35.72 | N |
| ATOM | 4578 | CA | ILE | B | 432 | 10.533 | 5.616 | 100.350 | 1.00 | 35.08 | C |
| ATOM | 4579 | C | ILE | B | 432 | 11.556 | 4.689 | 99.691 | 1.00 | 34.61 | C |
| ATOM | 4580 | O | ILE | B | 432 | 12.570 | 4.339 | 100.298 | 1.00 | 34.57 | O |
| ATOM | 4581 | CB | ILE | B | 432 | 11.056 | 7.064 | 100.489 | 1.00 | 35.06 | C |
| ATOM | 4582 | CG1 | ILE | B | 432 | 9.946 | 7.953 | 101.067 | 1.00 | 35.15 | C |
| ATOM | 4583 | CG2 | ILE | B | 432 | 11.542 | 7.596 | 99.141 | 1.00 | 34.85 | C |
| ATOM | 4584 | CD1 | ILE | B | 432 | 10.315 | 9.400 | 101.276 | 1.00 | 35.09 | C |
| ATOM | 4585 | N | TYR | B | 433 | 11.272 | 4.291 | 98.453 | 1.00 | 33.98 | N |
| ATOM | 4586 | CA | TYR | B | 433 | 12.040 | 3.247 | 97.779 | 1.00 | 33.66 | C |
| ATOM | 4587 | C | TYR | B | 433 | 12.103 | 3.430 | 96.259 | 1.00 | 33.46 | C |
| ATOM | 4588 | O | TYR | B | 433 | 11.307 | 4.170 | 95.687 | 1.00 | 33.49 | O |
| ATOM | 4589 | CB | TYR | B | 433 | 11.480 | 1.862 | 98.143 | 1.00 | 33.40 | C |
| ATOM | 4590 | CG | TYR | B | 433 | 9.983 | 1.751 | 98.008 | 1.00 | 33.48 | C |
| ATOM | 4591 | CD1 | TYR | B | 433 | 9.133 | 2.149 | 99.049 | 1.00 | 32.92 | C |
| ATOM | 4592 | CD2 | TYR | B | 433 | 9.408 | 1.250 | 96.840 | 1.00 | 33.35 | C |
| ATOM | 4593 | CE1 | TYR | B | 433 | 7.750 | 2.055 | 98.923 | 1.00 | 32.30 | C |
| ATOM | 4594 | CE2 | TYR | B | 433 | 8.024 | 1.149 | 96.707 | 1.00 | 33.17 | C |
| ATOM | 4595 | CZ | TYR | B | 433 | 7.205 | 1.552 | 97.752 | 1.00 | 32.81 | C |
| ATOM | 4596 | OH | TYR | B | 433 | 5.842 | 1.452 | 97.615 | 1.00 | 33.26 | O |
| ATOM | 4597 | N | ASP | B | 434 | 13.057 | 2.746 | 95.625 | 1.00 | 33.34 | N |
| ATOM | 4598 | CA | ASP | B | 434 | 13.297 | 2.837 | 94.181 | 1.00 | 33.25 | C |
| ATOM | 4599 | C | ASP | B | 434 | 12.318 | 2.000 | 93.366 | 1.00 | 33.38 | C |
| ATOM | 4600 | O | ASP | B | 434 | 11.755 | 1.024 | 93.863 | 1.00 | 33.52 | O |
| ATOM | 4601 | CB | ASP | B | 434 | 14.714 | 2.364 | 93.832 | 1.00 | 33.11 | C |
| ATOM | 4602 | CG | ASP | B | 434 | 15.801 | 3.189 | 94.495 | 1.00 | 32.44 | C |
| ATOM | 4603 | OD1 | ASP | B | 434 | 15.482 | 4.133 | 95.247 | 1.00 | 32.26 | O |
| ATOM | 4604 | OD2 | ASP | B | 434 | 16.985 | 2.887 | 94.252 | 1.00 | 31.33 | O |
| ATOM | 4605 | N | MET | B | 435 | 12.140 | 2.390 | 92.106 | 1.00 | 33.36 | N |
| ATOM | 4606 | CA | MET | B | 435 | 11.384 | 1.608 | 91.135 | 1.00 | 33.42 | C |
| ATOM | 4607 | C | MET | B | 435 | 12.000 | 1.701 | 89.738 | 1.00 | 33.41 | C |
| ATOM | 4608 | O | MET | B | 435 | 12.449 | 2.772 | 89.309 | 1.00 | 33.36 | O |
| ATOM | 4609 | CB | MET | B | 435 | 9.894 | 1.991 | 91.156 | 1.00 | 33.47 | C |
| ATOM | 4610 | CG | MET | B | 435 | 9.305 | 2.506 | 89.848 | 1.00 | 33.80 | C |
| ATOM | 4611 | SD | MET | B | 435 | 8.874 | 1.282 | 88.608 | 1.00 | 34.05 | S |
| ATOM | 4612 | CE | MET | B | 435 | 7.995 | 2.306 | 87.453 | 1.00 | 34.26 | C |
| ATOM | 4613 | N | PHE | B | 436 | 12.040 | 0.557 | 89.059 | 1.00 | 33.36 | N |
| ATOM | 4614 | CA | PHE | B | 436 | 12.452 | 0.459 | 87.664 | 1.00 | 33.44 | C |
| ATOM | 4615 | C | PHE | B | 436 | 11.429 | −0.431 | 86.964 | 1.00 | 33.66 | C |
| ATOM | 4616 | O | PHE | B | 436 | 10.896 | −1.350 | 87.586 | 1.00 | 33.75 | O |
| ATOM | 4617 | CB | PHE | B | 436 | 13.815 | −0.233 | 87.536 | 1.00 | 33.45 | C |
| ATOM | 4618 | CG | PHE | B | 436 | 14.945 | 0.463 | 88.245 | 1.00 | 33.20 | C |
| ATOM | 4619 | CD1 | PHE | B | 436 | 15.785 | 1.330 | 87.553 | 1.00 | 32.88 | C |
| ATOM | 4620 | CD2 | PHE | B | 436 | 15.203 | 0.210 | 89.592 | 1.00 | 33.08 | C |
| ATOM | 4621 | CE1 | PHE | B | 436 | 16.849 | 1.961 | 88.197 | 1.00 | 32.90 | C |
| ATOM | 4622 | CE2 | PHE | B | 436 | 16.263 | 0.839 | 90.250 | 1.00 | 33.05 | C |
| ATOM | 4623 | CZ | PHE | B | 436 | 17.086 | 1.717 | 89.552 | 1.00 | 33.19 | C |
| ATOM | 4624 | N | PRO | B | 437 | 11.147 | −0.173 | 85.672 | 1.00 | 33.93 | N |
| ATOM | 4625 | CA | PRO | B | 437 | 10.384 | −1.171 | 84.915 | 1.00 | 34.23 | C |
| ATOM | 4626 | C | PRO | B | 437 | 11.194 | −2.466 | 84.749 | 1.00 | 34.66 | C |
| ATOM | 4627 | O | PRO | B | 437 | 12.294 | −2.576 | 85.298 | 1.00 | 34.72 | O |
| ATOM | 4628 | CB | PRO | B | 437 | 10.141 | −0.486 | 83.560 | 1.00 | 34.19 | C |
| ATOM | 4629 | CG | PRO | B | 437 | 11.177 | 0.575 | 83.458 | 1.00 | 33.99 | C |
| ATOM | 4630 | CD | PRO | B | 437 | 11.485 | 1.011 | 84.857 | 1.00 | 33.99 | C |
| ATOM | 4631 | N | GLU | B | 438 | 10.648 | −3.432 | 84.012 | 1.00 | 35.09 | N |
| ATOM | 4632 | CA | GLU | B | 438 | 11.306 | −4.721 | 83.772 | 1.00 | 35.54 | C |
| ATOM | 4633 | C | GLU | B | 438 | 12.744 | −4.561 | 83.286 | 1.00 | 35.91 | C |
| ATOM | 4634 | O | GLU | B | 438 | 13.003 | −3.798 | 82.352 | 1.00 | 35.98 | O |
| ATOM | 4635 | CB | GLU | B | 438 | 10.504 | −5.532 | 82.749 | 1.00 | 35.53 | C |
| ATOM | 4636 | CG | GLU | B | 438 | 11.076 | −6.907 | 82.438 | 1.00 | 35.32 | C |
| ATOM | 4637 | CD | GLU | B | 438 | 10.459 | −7.543 | 81.207 | 1.00 | 35.70 | C |
| ATOM | 4638 | OE1 | GLU | B | 438 | 9.307 | −7.205 | 80.846 | 1.00 | 35.92 | O |
| ATOM | 4639 | OE2 | GLU | B | 438 | 11.133 | −8.393 | 80.596 | 1.00 | 36.39 | O |
| ATOM | 4640 | N | GLY | B | 439 | 13.669 | −5.282 | 83.918 | 1.00 | 36.29 | N |
| ATOM | 4641 | CA | GLY | B | 439 | 15.078 | −5.247 | 83.522 | 1.00 | 36.74 | C |
| ATOM | 4642 | C | GLY | B | 439 | 16.054 | −5.413 | 84.671 | 1.00 | 37.05 | C |
| ATOM | 4643 | O | GLY | B | 439 | 15.713 | −5.991 | 85.701 | 1.00 | 37.39 | O |
| ATOM | 4644 | N | LYS | B | 440 | 17.270 | −4.897 | 84.491 | 1.00 | 37.23 | N |
| ATOM | 4645 | CA | LYS | B | 440 | 18.347 | −5.063 | 85.467 | 1.00 | 37.34 | C |
| ATOM | 4646 | C | LYS | B | 440 | 18.514 | −3.858 | 86.396 | 1.00 | 37.39 | C |
| ATOM | 4647 | O | LYS | B | 440 | 19.603 | −3.632 | 86.932 | 1.00 | 37.44 | O |
| ATOM | 4648 | CB | LYS | B | 440 | 19.673 | −5.353 | 84.758 | 1.00 | 37.60 | C |
| ATOM | 4649 | CG | LYS | B | 440 | 19.750 | −6.708 | 84.066 | 1.00 | 38.31 | C |
| ATOM | 4650 | CD | LYS | B | 440 | 21.197 | −7.103 | 83.820 | 1.00 | 39.59 | C |
| ATOM | 4651 | CE | LYS | B | 440 | 21.304 | −8.437 | 83.095 | 1.00 | 40.85 | C |
| ATOM | 4652 | NZ | LYS | B | 440 | 22.727 | −8.871 | 82.959 | 1.00 | 41.75 | N |
| ATOM | 4653 | N | GLY | B | 441 | 17.432 | −3.103 | 86.584 | 1.00 | 37.36 | N |
| ATOM | 4654 | CA | GLY | B | 441 | 17.397 | −1.902 | 87.428 | 1.00 | 37.38 | C |
| ATOM | 4655 | C | GLY | B | 441 | 18.701 | −1.399 | 88.019 | 1.00 | 37.43 | C |
| ATOM | 4656 | O | GLY | B | 441 | 19.137 | −1.893 | 89.059 | 1.00 | 37.93 | O |
| ATOM | 4657 | N | ILE | B | 442 | 19.292 | −0.397 | 87.365 | 1.00 | 37.12 | N |
| ATOM | 4658 | CA | ILE | B | 442 | 20.606 | 0.217 | 87.695 | 1.00 | 36.85 | C |
| ATOM | 4659 | C | ILE | B | 442 | 21.714 | −0.322 | 86.799 | 1.00 | 36.60 | C |
| ATOM | 4660 | O | ILE | B | 442 | 22.546 | 0.445 | 86.314 | 1.00 | 36.66 | O |
| ATOM | 4661 | CB | ILE | B | 442 | 21.033 | 0.176 | 89.216 | 1.00 | 36.85 | C |
| ATOM | 4662 | CG1 | ILE | B | 442 | 22.172 | 1.158 | 89.495 | 1.00 | 36.91 | C |
| ATOM | 4663 | CG2 | ILE | B | 442 | 21.530 | −1.196 | 89.648 | 1.00 | 37.08 | C |
| ATOM | 4664 | CD1 | ILE | B | 442 | 21.737 | 2.522 | 89.916 | 1.00 | 36.32 | C |
| ATOM | 4665 | N | PHE | B | 443 | 21.714 | −1.636 | 86.581 | 1.00 | 36.28 | N |
| ATOM | 4666 | CA | PHE | B | 443 | 22.712 | −2.274 | 85.729 | 1.00 | 35.92 | C |
| ATOM | 4667 | C | PHE | B | 443 | 22.150 | −2.517 | 84.332 | 1.00 | 35.65 | C |
| ATOM | 4668 | O | PHE | B | 443 | 22.766 | −3.209 | 83.512 | 1.00 | 35.62 | O |
| ATOM | 4669 | CB | PHE | B | 443 | 23.218 | −3.580 | 86.361 | 1.00 | 35.91 | C |
| ATOM | 4670 | CG | PHE | B | 443 | 23.990 | −3.380 | 87.641 | 1.00 | 35.91 | C |
| ATOM | 4671 | CD1 | PHE | B | 443 | 25.203 | −2.692 | 87.642 | 1.00 | 35.97 | C |
| ATOM | 4672 | CD2 | PHE | B | 443 | 23.511 | −3.886 | 88.844 | 1.00 | 35.84 | C |
| ATOM | 4673 | CE1 | PHE | B | 443 | 25.918 | −2.503 | 88.824 | 1.00 | 36.17 | C |
| ATOM | 4674 | CE2 | PHE | B | 443 | 24.218 | −3.702 | 90.034 | 1.00 | 36.48 | C |
| ATOM | 4675 | CZ | PHE | B | 443 | 25.423 | −3.008 | 90.024 | 1.00 | 36.25 | C |
| ATOM | 4676 | N | GLY | B | 444 | 20.982 | −1.935 | 84.066 | 1.00 | 35.22 | N |
| ATOM | 4677 | CA | GLY | B | 444 | 20.315 | −2.098 | 82.780 | 1.00 | 34.80 | C |
| ATOM | 4678 | C | GLY | B | 444 | 19.541 | −0.875 | 82.338 | 1.00 | 34.37 | C |
| ATOM | 4679 | O | GLY | B | 444 | 18.461 | −1.001 | 81.763 | 1.00 | 34.54 | O |
| ATOM | 4680 | N | MET | B | 445 | 20.087 | 0.308 | 82.608 | 1.00 | 34.03 | N |
| ATOM | 4681 | CA | MET | B | 445 | 19.431 | 1.564 | 82.233 | 1.00 | 33.52 | C |
| ATOM | 4682 | C | MET | B | 445 | 19.783 | 1.952 | 80.799 | 1.00 | 33.70 | C |
| ATOM | 4683 | O | MET | B | 445 | 20.933 | 1.797 | 80.372 | 1.00 | 33.63 | O |
| ATOM | 4684 | CB | MET | B | 445 | 19.811 | 2.692 | 83.194 | 1.00 | 33.54 | C |
| ATOM | 4685 | CG | MET | B | 445 | 19.372 | 2.472 | 84.640 | 1.00 | 32.48 | C |
| ATOM | 4686 | SD | MET | B | 445 | 19.571 | 3.957 | 85.644 | 1.00 | 32.50 | S |
| ATOM | 4687 | CE | MET | B | 445 | 18.191 | 4.946 | 85.068 | 1.00 | 30.52 | C |
| ATOM | 4688 | N | SER | B | 446 | 18.787 | 2.455 | 80.066 | 1.00 | 33.53 | N |
| ATOM | 4689 | CA | SER | B | 446 | 18.972 | 2.849 | 78.669 | 1.00 | 33.44 | C |
| ATOM | 4690 | C | SER | B | 446 | 20.060 | 3.900 | 78.536 | 1.00 | 33.20 | C |
| ATOM | 4691 | O | SER | B | 446 | 20.272 | 4.708 | 79.442 | 1.00 | 33.38 | O |
| ATOM | 4692 | CB | SER | B | 446 | 17.663 | 3.358 | 78.058 | 1.00 | 33.62 | C |
| ATOM | 4693 | OG | SER | B | 446 | 17.243 | 4.574 | 78.662 | 1.00 | 34.60 | O |
| ATOM | 4694 | N | SER | B | 447 | 20.756 | 3.874 | 77.406 | 1.00 | 32.93 | N |
| ATOM | 4695 | CA | SER | B | 447 | 21.834 | 4.819 | 77.136 | 1.00 | 32.43 | C |
| ATOM | 4696 | C | SER | B | 447 | 21.303 | 6.232 | 76.860 | 1.00 | 31.90 | C |
| ATOM | 4697 | O | SER | B | 447 | 21.964 | 7.228 | 77.166 | 1.00 | 31.98 | O |
| ATOM | 4698 | CB | SER | B | 447 | 22.663 | 4.328 | 75.950 | 1.00 | 32.43 | C |
| ATOM | 4699 | OG | SER | B | 447 | 23.616 | 5.300 | 75.560 | 1.00 | 32.99 | O |
| ATOM | 4700 | N | GLN | B | 448 | 20.114 | 6.306 | 76.275 | 1.00 | 31.04 | N |
| ATOM | 4701 | CA | GLN | B | 448 | 19.528 | 7.580 | 75.882 | 1.00 | 30.44 | C |
| ATOM | 4702 | C | GLN | B | 448 | 18.304 | 7.935 | 76.734 | 1.00 | 30.21 | C |
| ATOM | 4703 | O | GLN | B | 448 | 17.587 | 7.051 | 77.222 | 1.00 | 29.99 | O |
| ATOM | 4704 | CB | GLN | B | 448 | 19.171 | 7.557 | 74.390 | 1.00 | 30.25 | C |
| ATOM | 4705 | CG | GLN | B | 448 | 20.369 | 7.357 | 73.452 | 1.00 | 29.72 | C |
| ATOM | 4706 | CD | GLN | B | 448 | 21.385 | 8.485 | 73.523 | 1.00 | 29.84 | C |
| ATOM | 4707 | OE1 | GLN | B | 448 | 21.045 | 9.664 | 73.376 | 1.00 | 29.89 | O |
| ATOM | 4708 | NE2 | GLN | B | 448 | 22.640 | 8.127 | 73.749 | 1.00 | 28.51 | N |
| ATOM | 4709 | N | LYS | B | 449 | 18.073 | 9.230 | 76.916 | 1.00 | 29.98 | N |
| ATOM | 4710 | CA | LYS | B | 449 | 16.954 | 9.686 | 77.738 | 1.00 | 29.95 | C |
| ATOM | 4711 | C | LYS | B | 449 | 15.608 | 9.537 | 77.038 | 1.00 | 29.80 | C |
| ATOM | 4712 | O | LYS | B | 449 | 15.438 | 9.939 | 75.890 | 1.00 | 29.38 | O |
| ATOM | 4713 | CB | LYS | B | 449 | 17.138 | 11.137 | 78.197 | 1.00 | 29.83 | C |
| ATOM | 4714 | CG | LYS | B | 449 | 16.032 | 11.607 | 79.130 | 1.00 | 30.25 | C |
| ATOM | 4715 | CD | LYS | B | 449 | 16.163 | 13.067 | 79.497 | 1.00 | 31.70 | C |
| ATOM | 4716 | CE | LYS | B | 449 | 15.244 | 13.416 | 80.672 | 1.00 | 32.38 | C |
| ATOM | 4717 | NZ | LYS | B | 449 | 15.728 | 12.839 | 81.963 | 1.00 | 32.40 | N |
| ATOM | 4718 | N | GLU | B | 450 | 14.672 | 8.929 | 77.754 | 1.00 | 29.99 | N |
| ATOM | 4719 | CA | GLU | B | 450 | 13.263 | 8.988 | 77.420 | 1.00 | 30.50 | C |
| ATOM | 4720 | C | GLU | B | 450 | 12.602 | 9.872 | 78.465 | 1.00 | 30.16 | C |
| ATOM | 4721 | O | GLU | B | 450 | 12.758 | 9.638 | 79.658 | 1.00 | 29.76 | O |
| ATOM | 4722 | CB | GLU | B | 450 | 12.656 | 7.584 | 77.438 | 1.00 | 30.48 | C |
| ATOM | 4723 | CG | GLU | B | 450 | 13.186 | 6.663 | 76.336 | 1.00 | 31.38 | C |
| ATOM | 4724 | CD | GLU | B | 450 | 12.707 | 5.221 | 76.468 | 1.00 | 31.45 | C |
| ATOM | 4725 | OE1 | GLU | B | 450 | 11.728 | 4.967 | 77.200 | 1.00 | 33.27 | O |
| ATOM | 4726 | OE2 | GLU | B | 450 | 13.313 | 4.332 | 75.836 | 1.00 | 32.81 | O |
| ATOM | 4727 | N | GLU | B | 451 | 11.897 | 10.910 | 78.025 | 1.00 | 30.50 | N |
| ATOM | 4728 | CA | GLU | B | 451 | 11.148 | 11.755 | 78.960 | 1.00 | 30.86 | C |
| ATOM | 4729 | C | GLU | B | 451 | 10.071 | 10.917 | 79.639 | 1.00 | 30.54 | C |
| ATOM | 4730 | O | GLU | B | 451 | 9.367 | 10.155 | 78.984 | 1.00 | 30.46 | O |
| ATOM | 4731 | CB | GLU | B | 451 | 10.532 | 12.953 | 78.245 | 1.00 | 31.09 | C |
| ATOM | 4732 | CG | GLU | B | 451 | 11.487 | 14.124 | 78.062 | 1.00 | 33.25 | C |
| ATOM | 4733 | CD | GLU | B | 451 | 11.411 | 14.725 | 76.671 | 1.00 | 35.64 | C |
| ATOM | 4734 | OE1 | GLU | B | 451 | 10.370 | 14.561 | 75.997 | 1.00 | 37.37 | O |
| ATOM | 4735 | OE2 | GLU | B | 451 | 12.397 | 15.359 | 76.242 | 1.00 | 36.47 | O |
| ATOM | 4736 | N | ALA | B | 452 | 9.972 | 11.040 | 80.956 | 1.00 | 30.40 | N |
| ATOM | 4737 | CA | ALA | B | 452 | 9.052 | 10.217 | 81.733 | 1.00 | 30.30 | C |
| ATOM | 4738 | C | ALA | B | 452 | 7.769 | 10.960 | 82.095 | 1.00 | 30.37 | C |
| ATOM | 4739 | O | ALA | B | 452 | 6.736 | 10.336 | 82.351 | 1.00 | 30.33 | O |
| ATOM | 4740 | CB | ALA | B | 452 | 9.744 | 9.691 | 82.984 | 1.00 | 30.05 | C |
| ATOM | 4741 | N | TYR | B | 453 | 7.846 | 12.287 | 82.128 | 1.00 | 30.43 | N |
| ATOM | 4742 | CA | TYR | B | 453 | 6.715 | 13.119 | 82.525 | 1.00 | 30.99 | C |
| ATOM | 4743 | C | TYR | B | 453 | 6.550 | 14.320 | 81.614 | 1.00 | 31.27 | C |
| ATOM | 4744 | O | TYR | B | 453 | 7.487 | 14.729 | 80.932 | 1.00 | 31.25 | O |
| ATOM | 4745 | CB | TYR | B | 453 | 6.864 | 13.592 | 83.976 | 1.00 | 30.93 | C |
| ATOM | 4746 | CG | TYR | B | 453 | 7.074 | 12.472 | 84.957 | 1.00 | 31.11 | C |
| ATOM | 4747 | CD1 | TYR | B | 453 | 8.361 | 12.067 | 85.309 | 1.00 | 30.25 | C |
| ATOM | 4748 | CD2 | TYR | B | 453 | 5.987 | 11.803 | 85.527 | 1.00 | 30.70 | C |
| ATOM | 4749 | CE1 | TYR | B | 453 | 8.563 | 11.030 | 86.198 | 1.00 | 30.30 | C |
| ATOM | 4750 | CE2 | TYR | B | 453 | 6.182 | 10.764 | 86.431 | 1.00 | 30.41 | C |
| ATOM | 4751 | CZ | TYR | B | 453 | 7.474 | 10.386 | 86.758 | 1.00 | 30.49 | C |
| ATOM | 4752 | OH | TYR | B | 453 | 7.690 | 9.363 | 87.646 | 1.00 | 31.20 | O |
| ATOM | 4753 | N | LYS | B | 454 | 5.354 | 14.892 | 81.626 | 1.00 | 31.89 | N |
| ATOM | 4754 | CA | LYS | B | 454 | 5.025 | 16.001 | 80.750 | 1.00 | 32.58 | C |
| ATOM | 4755 | C | LYS | B | 454 | 3.950 | 16.879 | 81.394 | 1.00 | 33.26 | C |
| ATOM | 4756 | O | LYS | B | 454 | 2.910 | 16.379 | 81.823 | 1.00 | 33.15 | O |
| ATOM | 4757 | CB | LYS | B | 454 | 4.561 | 15.438 | 79.402 | 1.00 | 32.49 | C |
| ATOM | 4758 | CG | LYS | B | 454 | 4.107 | 16.452 | 78.384 | 1.00 | 32.56 | C |
| ATOM | 4759 | CD | LYS | B | 454 | 3.977 | 15.807 | 77.022 | 1.00 | 31.87 | C |
| ATOM | 4760 | CE | LYS | B | 454 | 3.069 | 16.622 | 76.116 | 1.00 | 32.11 | C |
| ATOM | 4761 | NZ | LYS | B | 454 | 3.465 | 18.049 | 76.012 | 1.00 | 31.33 | N |
| ATOM | 4762 | N | LYS | B | 455 | 4.211 | 18.185 | 81.456 | 1.00 | 34.32 | N |
| ATOM | 4763 | CA | LYS | B | 455 | 3.249 | 19.160 | 81.990 | 1.00 | 35.28 | C |
| ATOM | 4764 | C | LYS | B | 455 | 2.273 | 19.646 | 80.927 | 1.00 | 35.80 | C |
| ATOM | 4765 | O | LYS | B | 455 | 2.685 | 20.083 | 79.852 | 1.00 | 35.93 | O |
| ATOM | 4766 | CB | LYS | B | 455 | 3.965 | 20.362 | 82.605 | 1.00 | 35.33 | C |
| ATOM | 4767 | CG | LYS | B | 455 | 4.766 | 20.042 | 83.850 | 1.00 | 36.66 | C |
| ATOM | 4768 | CD | LYS | B | 455 | 5.255 | 21.301 | 84.557 | 1.00 | 38.54 | C |
| ATOM | 4769 | CE | LYS | B | 455 | 4.135 | 21.974 | 85.340 | 1.00 | 39.34 | C |
| ATOM | 4770 | NZ | LYS | B | 455 | 4.666 | 22.850 | 86.426 | 1.00 | 40.66 | N |
| ATOM | 4771 | N | ILE | B | 456 | 0.980 | 19.559 | 81.238 | 1.00 | 36.57 | N |
| ATOM | 4772 | CA | ILE | B | 456 | −0.091 | 20.028 | 80.348 | 1.00 | 37.07 | C |
| ATOM | 4773 | C | ILE | B | 456 | −1.124 | 20.807 | 81.170 | 1.00 | 37.88 | C |
| ATOM | 4774 | O | ILE | B | 456 | −1.849 | 20.221 | 81.986 | 1.00 | 37.96 | O |
| ATOM | 4775 | CB | ILE | B | 456 | −0.770 | 18.857 | 79.598 | 1.00 | 36.98 | C |
| ATOM | 4776 | CG1 | ILE | B | 456 | 0.270 | 18.072 | 78.779 | 1.00 | 36.71 | C |
| ATOM | 4777 | CG2 | ILE | B | 456 | −1.910 | 19.384 | 78.711 | 1.00 | 36.71 | C |
| ATOM | 4778 | CD1 | ILE | B | 456 | −0.165 | 16.689 | 78.328 | 1.00 | 36.48 | C |
| ATOM | 4779 | N | ASP | B | 457 | −1.186 | 22.123 | 80.944 | 1.00 | 38.64 | N |
| ATOM | 4780 | CA | ASP | B | 457 | −1.851 | 23.062 | 81.867 | 1.00 | 39.37 | C |
| ATOM | 4781 | C | ASP | B | 457 | −1.327 | 22.769 | 83.265 | 1.00 | 39.46 | C |
| ATOM | 4782 | O | ASP | B | 457 | −2.039 | 22.916 | 84.257 | 1.00 | 39.56 | O |
| ATOM | 4783 | CB | ASP | B | 457 | −3.376 | 22.923 | 81.813 | 1.00 | 39.57 | C |
| ATOM | 4784 | CG | ASP | B | 457 | −3.917 | 22.972 | 80.398 | 1.00 | 40.79 | C |
| ATOM | 4785 | OD1 | ASP | B | 457 | −3.128 | 23.241 | 79.461 | 1.00 | 42.03 | O |
| ATOM | 4786 | OD2 | ASP | B | 457 | −5.133 | 22.740 | 80.219 | 1.00 | 42.20 | O |
| ATOM | 4787 | N | GLY | B | 458 | −0.052 | 22.381 | 83.298 | 1.00 | 39.55 | N |
| ATOM | 4788 | CA | GLY | B | 458 | 0.573 | 21.615 | 84.374 | 1.00 | 39.46 | C |
| ATOM | 4789 | C | GLY | B | 458 | 0.073 | 21.788 | 85.789 | 1.00 | 39.17 | C |
| ATOM | 4790 | O | GLY | B | 458 | 0.055 | 22.911 | 86.279 | 1.00 | 39.56 | O |
| ATOM | 4791 | N | LYS | B | 459 | −0.352 | 20.714 | 86.466 | 1.00 | 38.81 | N |
| ATOM | 4792 | CA | LYS | B | 459 | −0.598 | 19.340 | 85.944 | 1.00 | 38.18 | C |
| ATOM | 4793 | C | LYS | B | 459 | 0.530 | 18.567 | 85.236 | 1.00 | 37.41 | C |
| ATOM | 4794 | O | LYS | B | 459 | 0.747 | 18.697 | 84.029 | 1.00 | 37.24 | O |
| ATOM | 4795 | CB | LYS | B | 459 | −1.910 | 19.260 | 85.149 | 1.00 | 38.40 | C |
| ATOM | 4796 | CG | LYS | B | 459 | −3.164 | 19.382 | 86.004 | 1.00 | 39.14 | C |
| ATOM | 4797 | CD | LYS | B | 459 | −4.378 | 19.701 | 85.140 | 1.00 | 40.66 | C |
| ATOM | 4798 | CE | LYS | B | 459 | −5.593 | 20.074 | 85.979 | 1.00 | 41.66 | C |
| ATOM | 4799 | NZ | LYS | B | 459 | −6.784 | 20.335 | 85.109 | 1.00 | 42.45 | N |
| ATOM | 4800 | N | THR | B | 460 | 1.213 | 17.734 | 86.013 | 1.00 | 36.61 | N |
| ATOM | 4801 | CA | THR | B | 460 | 2.243 | 16.843 | 85.505 | 1.00 | 35.78 | C |
| ATOM | 4802 | C | THR | B | 460 | 1.669 | 15.437 | 85.356 | 1.00 | 35.31 | C |
| ATOM | 4803 | O | THR | B | 460 | 1.131 | 14.884 | 86.315 | 1.00 | 35.25 | O |
| ATOM | 4804 | CB | THR | B | 460 | 3.460 | 16.799 | 86.466 | 1.00 | 35.88 | C |
| ATOM | 4805 | OG1 | THR | B | 460 | 3.802 | 18.131 | 86.874 | 1.00 | 35.23 | O |
| ATOM | 4806 | CG2 | THR | B | 460 | 4.663 | 16.134 | 85.799 | 1.00 | 35.20 | C |
| ATOM | 4807 | N | TYR | B | 461 | 1.779 | 14.869 | 84.155 | 1.00 | 34.57 | N |
| ATOM | 4808 | CA | TYR | B | 461 | 1.353 | 13.491 | 83.898 | 1.00 | 34.01 | C |
| ATOM | 4809 | C | TYR | B | 461 | 2.537 | 12.599 | 83.527 | 1.00 | 33.40 | C |
| ATOM | 4810 | O | TYR | B | 461 | 3.538 | 13.083 | 82.997 | 1.00 | 33.22 | O |
| ATOM | 4811 | CB | TYR | B | 461 | 0.335 | 13.432 | 82.754 | 1.00 | 34.36 | C |
| ATOM | 4812 | CG | TYR | B | 461 | −0.893 | 14.290 | 82.919 | 1.00 | 34.82 | C |
| ATOM | 4813 | CD1 | TYR | B | 461 | −1.894 | 13.947 | 83.829 | 1.00 | 35.48 | C |
| ATOM | 4814 | CD2 | TYR | B | 461 | −1.071 | 15.433 | 82.144 | 1.00 | 35.18 | C |
| ATOM | 4815 | CE1 | TYR | B | 461 | −3.039 | 14.730 | 83.975 | 1.00 | 35.41 | C |
| ATOM | 4816 | CE2 | TYR | B | 461 | −2.209 | 16.226 | 82.285 | 1.00 | 35.95 | C |
| ATOM | 4817 | CZ | TYR | B | 461 | −3.190 | 15.865 | 83.199 | 1.00 | 35.55 | C |
| ATOM | 4818 | OH | TYR | B | 461 | −4.318 | 16.641 | 83.335 | 1.00 | 35.74 | O |
| ATOM | 4819 | N | GLN | B | 462 | 2.405 | 11.296 | 83.779 | 1.00 | 32.74 | N |
| ATOM | 4820 | CA | GLN | B | 462 | 3.404 | 10.323 | 83.332 | 1.00 | 32.07 | C |
| ATOM | 4821 | C | GLN | B | 462 | 3.195 | 9.932 | 81.869 | 1.00 | 31.52 | C |
| ATOM | 4822 | O | GLN | B | 462 | 2.067 | 9.696 | 81.436 | 1.00 | 31.54 | O |
| ATOM | 4823 | CB | GLN | B | 462 | 3.430 | 9.076 | 84.227 | 1.00 | 32.05 | C |
| ATOM | 4824 | CG | GLN | B | 462 | 4.485 | 8.047 | 83.783 | 1.00 | 32.29 | C |
| ATOM | 4825 | CD | GLN | B | 462 | 4.782 | 6.975 | 84.816 | 1.00 | 32.12 | C |
| ATOM | 4826 | OE1 | GLN | B | 462 | 3.886 | 6.469 | 85.496 | 1.00 | 32.22 | O |
| ATOM | 4827 | NE2 | GLN | B | 462 | 6.050 | 6.609 | 84.921 | 1.00 | 31.17 | N |
| ATOM | 4828 | N | ILE | B | 463 | 4.298 | 9.864 | 81.126 | 1.00 | 30.80 | N |
| ATOM | 4829 | CA | ILE | B | 463 | 4.284 | 9.542 | 79.702 | 1.00 | 29.98 | C |
| ATOM | 4830 | C | ILE | B | 463 | 4.232 | 8.031 | 79.448 | 1.00 | 29.91 | C |
| ATOM | 4831 | O | ILE | B | 463 | 4.788 | 7.234 | 80.210 | 1.00 | 29.63 | O |
| ATOM | 4832 | CB | ILE | B | 463 | 5.517 | 10.159 | 78.973 | 1.00 | 29.94 | C |
| ATOM | 4833 | CG1 | ILE | B | 463 | 5.475 | 11.694 | 79.047 | 1.00 | 29.77 | C |
| ATOM | 4834 | CG2 | ILE | B | 463 | 5.604 | 9.676 | 77.521 | 1.00 | 28.94 | C |
| ATOM | 4835 | CD1 | ILE | B | 463 | 6.773 | 12.383 | 78.647 | 1.00 | 29.46 | C |
| ATOM | 4836 | N | LEU | B | 464 | 3.539 | 7.659 | 78.375 | 1.00 | 29.71 | N |
| ATOM | 4837 | CA | LEU | B | 464 | 3.570 | 6.309 | 77.838 | 1.00 | 29.69 | C |
| ATOM | 4838 | C | LEU | B | 464 | 4.056 | 6.368 | 76.404 | 1.00 | 29.90 | C |
| ATOM | 4839 | O | LEU | B | 464 | 3.686 | 7.268 | 75.663 | 1.00 | 29.74 | O |
| ATOM | 4840 | CB | LEU | B | 464 | 2.180 | 5.683 | 77.849 | 1.00 | 29.33 | C |
| ATOM | 4841 | CG | LEU | B | 464 | 1.568 | 5.259 | 79.176 | 1.00 | 29.13 | C |
| ATOM | 4842 | CD1 | LEU | B | 464 | 0.138 | 4.790 | 78.935 | 1.00 | 27.98 | C |
| ATOM | 4843 | CD2 | LEU | B | 464 | 2.397 | 4.170 | 79.842 | 1.00 | 27.91 | C |
| ATOM | 4844 | N | TYR | B | 465 | 4.882 | 5.411 | 76.009 | 1.00 | 30.23 | N |
| ATOM | 4845 | CA | TYR | B | 465 | 5.259 | 5.291 | 74.607 | 1.00 | 30.74 | C |
| ATOM | 4846 | C | TYR | B | 465 | 4.544 | 4.100 | 73.985 | 1.00 | 31.47 | C |
| ATOM | 4847 | O | TYR | B | 465 | 4.414 | 3.043 | 74.614 | 1.00 | 31.49 | O |
| ATOM | 4848 | CB | TYR | B | 465 | 6.777 | 5.181 | 74.455 | 1.00 | 30.18 | C |
| ATOM | 4849 | CG | TYR | B | 465 | 7.498 | 6.366 | 75.037 | 1.00 | 29.58 | C |
| ATOM | 4850 | CD1 | TYR | B | 465 | 7.651 | 7.541 | 74.301 | 1.00 | 29.46 | C |
| ATOM | 4851 | CD2 | TYR | B | 465 | 7.996 | 6.332 | 76.336 | 1.00 | 28.89 | C |
| ATOM | 4852 | CE1 | TYR | B | 465 | 8.301 | 8.644 | 74.839 | 1.00 | 28.84 | C |
| ATOM | 4853 | CE2 | TYR | B | 465 | 8.647 | 7.429 | 76.881 | 1.00 | 28.47 | C |
| ATOM | 4854 | CZ | TYR | B | 465 | 8.797 | 8.580 | 76.128 | 1.00 | 28.72 | C |
| ATOM | 4855 | OH | TYR | B | 465 | 9.437 | 9.675 | 76.662 | 1.00 | 29.00 | O |
| ATOM | 4856 | N | ARG | B | 466 | 4.056 | 4.294 | 72.763 | 1.00 | 32.45 | N |
| ATOM | 4857 | CA | ARG | B | 466 | 3.381 | 3.237 | 72.030 | 1.00 | 33.40 | C |
| ATOM | 4858 | C | ARG | B | 466 | 4.256 | 2.657 | 70.927 | 1.00 | 34.15 | C |
| ATOM | 4859 | O | ARG | B | 466 | 4.791 | 3.395 | 70.095 | 1.00 | 34.29 | O |
| ATOM | 4860 | CB | ARG | B | 466 | 2.059 | 3.717 | 71.440 | 1.00 | 33.33 | C |
| ATOM | 4861 | CG | ARG | B | 466 | 1.193 | 2.557 | 71.021 | 1.00 | 34.23 | C |
| ATOM | 4862 | CD | ARG | B | 466 | 0.012 | 2.942 | 70.177 | 1.00 | 35.97 | C |
| ATOM | 4863 | NE | ARG | B | 466 | −0.633 | 1.724 | 69.697 | 1.00 | 36.79 | N |
| ATOM | 4864 | CZ | ARG | B | 466 | −1.940 | 1.570 | 69.514 | 1.00 | 37.11 | C |
| ATOM | 4865 | NH1 | ARG | B | 466 | −2.783 | 2.567 | 69.767 | 1.00 | 37.21 | N |
| ATOM | 4866 | NH2 | ARG | B | 466 | −2.402 | 0.402 | 69.085 | 1.00 | 37.38 | N |
| ATOM | 4867 | N | GLU | B | 467 | 4.381 | 1.330 | 70.936 | 1.00 | 34.93 | N |
| ATOM | 4868 | CA | GLU | B | 467 | 5.103 | 0.581 | 69.915 | 1.00 | 35.83 | C |
| ATOM | 4869 | C | GLU | B | 467 | 4.826 | 1.103 | 68.503 | 1.00 | 35.98 | C |
| ATOM | 4870 | O | GLU | B | 467 | 3.688 | 1.067 | 68.036 | 1.00 | 36.09 | O |
| ATOM | 4871 | CB | GLU | B | 467 | 4.726 | −0.903 | 70.014 | 1.00 | 36.19 | C |
| ATOM | 4872 | CG | GLU | B | 467 | 5.314 | −1.802 | 68.918 | 1.00 | 38.01 | C |
| ATOM | 4873 | CD | GLU | B | 467 | 6.831 | −1.939 | 68.997 | 1.00 | 40.27 | C |
| ATOM | 4874 | OE1 | GLU | B | 467 | 7.423 | −1.590 | 70.047 | 1.00 | 41.05 | O |
| ATOM | 4875 | OE2 | GLU | B | 467 | 7.432 | −2.402 | 68.001 | 1.00 | 41.08 | O |
| ATOM | 4876 | N | GLY | B | 468 | 5.873 | 1.591 | 67.840 | 1.00 | 36.19 | N |
| ATOM | 4877 | CA | GLY | B | 468 | 5.777 | 2.054 | 66.455 | 1.00 | 36.30 | C |
| ATOM | 4878 | C | GLY | B | 468 | 5.671 | 3.558 | 66.287 | 1.00 | 36.54 | C |
| ATOM | 4879 | O | GLY | B | 468 | 5.831 | 4.078 | 65.180 | 1.00 | 36.46 | O |
| ATOM | 4880 | N | GLU | B | 469 | 5.414 | 4.262 | 67.386 | 1.00 | 36.66 | N |
| ATOM | 4881 | CA | GLU | B | 469 | 5.095 | 5.686 | 67.327 | 1.00 | 36.87 | C |
| ATOM | 4882 | C | GLU | B | 469 | 6.022 | 6.539 | 68.173 | 1.00 | 36.60 | C |
| ATOM | 4883 | O | GLU | B | 469 | 6.403 | 6.164 | 69.281 | 1.00 | 36.69 | O |
| ATOM | 4884 | CB | GLU | B | 469 | 3.651 | 5.941 | 67.782 | 1.00 | 36.79 | C |
| ATOM | 4885 | CG | GLU | B | 469 | 2.573 | 5.369 | 66.871 | 1.00 | 37.40 | C |
| ATOM | 4886 | CD | GLU | B | 469 | 1.192 | 5.345 | 67.525 | 1.00 | 37.81 | C |
| ATOM | 4887 | OE1 | GLU | B | 469 | 0.831 | 6.325 | 68.231 | 1.00 | 37.58 | O |
| ATOM | 4888 | OE2 | GLU | B | 469 | 0.465 | 4.340 | 67.320 | 1.00 | 38.70 | O |
| ATOM | 4889 | N | LYS | B | 470 | 6.373 | 7.696 | 67.633 | 1.00 | 36.48 | N |
| ATOM | 4890 | CA | LYS | B | 470 | 6.975 | 8.757 | 68.413 | 1.00 | 36.30 | C |
| ATOM | 4891 | C | LYS | B | 470 | 5.826 | 9.631 | 68.916 | 1.00 | 36.13 | C |
| ATOM | 4892 | O | LYS | B | 470 | 4.807 | 9.782 | 68.238 | 1.00 | 36.19 | O |
| ATOM | 4893 | CB | LYS | B | 470 | 7.926 | 9.566 | 67.534 | 1.00 | 36.19 | C |
| ATOM | 4894 | CG | LYS | B | 470 | 8.718 | 10.625 | 68.260 | 1.00 | 36.27 | C |
| ATOM | 4895 | CD | LYS | B | 470 | 9.273 | 11.640 | 67.279 | 1.00 | 36.32 | C |
| ATOM | 4896 | CE | LYS | B | 470 | 9.785 | 12.870 | 68.006 | 1.00 | 36.29 | C |
| ATOM | 4897 | NZ | LYS | B | 470 | 10.563 | 13.752 | 67.100 | 1.00 | 36.45 | N |
| ATOM | 4898 | N | GLY | B | 471 | 5.975 | 10.198 | 70.104 | 1.00 | 35.90 | N |
| ATOM | 4899 | CA | GLY | B | 471 | 4.942 | 11.075 | 70.634 | 1.00 | 35.64 | C |
| ATOM | 4900 | C | GLY | B | 471 | 4.578 | 10.719 | 72.055 | 1.00 | 35.48 | C |
| ATOM | 4901 | O | GLY | B | 471 | 4.726 | 9.566 | 72.466 | 1.00 | 35.30 | O |
| ATOM | 4902 | N | ALA | B | 472 | 4.094 | 11.720 | 72.790 | 1.00 | 35.40 | N |
| ATOM | 4903 | CA | ALA | B | 472 | 3.771 | 11.595 | 74.211 | 1.00 | 35.25 | C |
| ATOM | 4904 | C | ALA | B | 472 | 2.863 | 10.412 | 74.543 | 1.00 | 35.06 | C |
| ATOM | 4905 | O | ALA | B | 472 | 3.354 | 9.306 | 74.740 | 1.00 | 35.26 | O |
| ATOM | 4906 | CB | ALA | B | 472 | 3.173 | 12.898 | 74.744 | 1.00 | 35.50 | C |
| ATOM | 4907 | N | TYR | B | 473 | 1.550 | 10.636 | 74.570 | 1.00 | 34.61 | N |
| ATOM | 4908 | CA | TYR | B | 473 | 0.609 | 9.740 | 75.264 | 1.00 | 34.04 | C |
| ATOM | 4909 | C | TYR | B | 473 | 0.791 | 9.975 | 76.752 | 1.00 | 33.49 | C |
| ATOM | 4910 | O | TYR | B | 473 | 1.896 | 9.818 | 77.281 | 1.00 | 33.38 | O |
| ATOM | 4911 | CB | TYR | B | 473 | 0.835 | 8.252 | 74.956 | 1.00 | 34.06 | C |
| ATOM | 4912 | CG | TYR | B | 473 | 0.309 | 7.770 | 73.626 | 1.00 | 34.41 | C |
| ATOM | 4913 | CD1 | TYR | B | 473 | −1.061 | 7.588 | 73.417 | 1.00 | 34.80 | C |
| ATOM | 4914 | CD2 | TYR | B | 473 | 1.181 | 7.460 | 72.584 | 1.00 | 34.04 | C |
| ATOM | 4915 | CE1 | TYR | B | 473 | −1.547 | 7.136 | 72.190 | 1.00 | 34.66 | C |
| ATOM | 4916 | CE2 | TYR | B | 473 | 0.709 | 7.011 | 71.363 | 1.00 | 34.05 | C |
| ATOM | 4917 | CZ | TYR | B | 473 | −0.654 | 6.849 | 71.169 | 1.00 | 34.50 | C |
| ATOM | 4918 | OH | TYR | B | 473 | −1.123 | 6.398 | 69.955 | 1.00 | 34.72 | O |
| ATOM | 4919 | N | THR | B | 474 | −0.289 | 10.359 | 77.423 | 1.00 | 32.90 | N |
| ATOM | 4920 | CA | THR | B | 474 | −0.226 | 10.685 | 78.847 | 1.00 | 32.46 | C |
| ATOM | 4921 | C | THR | B | 474 | −1.267 | 9.925 | 79.664 | 1.00 | 32.34 | C |
| ATOM | 4922 | O | THR | B | 474 | −2.323 | 9.541 | 79.149 | 1.00 | 32.27 | O |
| ATOM | 4923 | CB | THR | B | 474 | −0.366 | 12.208 | 79.095 | 1.00 | 32.36 | C |
| ATOM | 4924 | OG1 | THR | B | 474 | −1.451 | 12.732 | 78.317 | 1.00 | 32.38 | O |
| ATOM | 4925 | CG2 | THR | B | 474 | 0.921 | 12.935 | 78.716 | 1.00 | 31.91 | C |
| ATOM | 4926 | N | ILE | B | 475 | −0.945 | 9.690 | 80.932 | 1.00 | 32.13 | N |
| ATOM | 4927 | CA | ILE | B | 475 | −1.875 | 9.071 | 81.865 | 1.00 | 32.23 | C |
| ATOM | 4928 | C | ILE | B | 475 | −2.573 | 10.182 | 82.643 | 1.00 | 32.32 | C |
| ATOM | 4929 | O | ILE | B | 475 | −1.971 | 10.785 | 83.531 | 1.00 | 32.34 | O |
| ATOM | 4930 | CB | ILE | B | 475 | −1.155 | 8.112 | 82.850 | 1.00 | 32.18 | C |
| ATOM | 4931 | CG1 | ILE | B | 475 | −0.360 | 7.047 | 82.086 | 1.00 | 32.06 | C |
| ATOM | 4932 | CG2 | ILE | B | 475 | −2.159 | 7.476 | 83.805 | 1.00 | 32.01 | C |
| ATOM | 4933 | CD1 | ILE | B | 475 | 0.395 | 6.082 | 82.966 | 1.00 | 32.32 | C |
| ATOM | 4934 | N | ARG | B | 476 | −3.835 | 10.455 | 82.307 | 1.00 | 32.44 | N |
| ATOM | 4935 | CA | ARG | B | 476 | −4.577 | 11.541 | 82.961 | 1.00 | 32.75 | C |
| ATOM | 4936 | C | ARG | B | 476 | −5.585 | 11.033 | 84.000 | 1.00 | 32.95 | C |
| ATOM | 4937 | O | ARG | B | 476 | −5.357 | 9.984 | 84.607 | 1.00 | 32.95 | O |
| ATOM | 4938 | CB | ARG | B | 476 | −5.200 | 12.485 | 81.926 | 1.00 | 32.71 | C |
| ATOM | 4939 | CG | ARG | B | 476 | −4.186 | 12.954 | 80.884 | 1.00 | 32.71 | C |
| ATOM | 4940 | CD | ARG | B | 476 | −4.686 | 14.131 | 80.074 | 1.00 | 33.01 | C |
| ATOM | 4941 | NE | ARG | B | 476 | −3.853 | 14.372 | 78.894 | 1.00 | 31.63 | N |
| ATOM | 4942 | CZ | ARG | B | 476 | −4.011 | 15.403 | 78.070 | 1.00 | 31.97 | C |
| ATOM | 4943 | NH1 | ARG | B | 476 | −4.971 | 16.297 | 78.299 | 1.00 | 32.00 | N |
| ATOM | 4944 | NH2 | ARG | B | 476 | −3.211 | 15.548 | 77.020 | 1.00 | 31.07 | N |
| ATOM | 4945 | N | GLU | B | 477 | −6.677 | 11.767 | 84.217 | 1.00 | 33.20 | N |
| ATOM | 4946 | CA | GLU | B | 477 | −7.625 | 11.447 | 85.300 | 1.00 | 33.56 | C |
| ATOM | 4947 | C | GLU | B | 477 | −8.135 | 10.007 | 85.247 | 1.00 | 33.45 | C |
| ATOM | 4948 | O | GLU | B | 477 | −8.388 | 9.468 | 84.168 | 1.00 | 33.34 | O |
| ATOM | 4949 | CB | GLU | B | 477 | −8.800 | 12.440 | 85.337 | 1.00 | 33.50 | C |
| ATOM | 4950 | CG | GLU | B | 477 | −9.832 | 12.286 | 84.207 | 1.00 | 34.47 | C |
| ATOM | 4951 | CD | GLU | B | 477 | −9.298 | 12.687 | 82.836 | 1.00 | 35.65 | C |
| ATOM | 4952 | OE1 | GLU | B | 477 | −8.414 | 13.579 | 82.761 | 1.00 | 36.24 | O |
| ATOM | 4953 | OE2 | GLU | B | 477 | −9.767 | 12.108 | 81.831 | 1.00 | 35.33 | O |
| ATOM | 4954 | N | ASN | B | 478 | −8.262 | 9.396 | 86.425 | 1.00 | 33.66 | N |
| ATOM | 4955 | CA | ASN | B | 478 | −8.732 | 8.008 | 86.587 | 1.00 | 33.93 | C |
| ATOM | 4956 | C | ASN | B | 478 | −7.885 | 6.971 | 85.847 | 1.00 | 34.03 | C |
| ATOM | 4957 | O | ASN | B | 478 | −8.287 | 5.816 | 85.706 | 1.00 | 33.98 | O |
| ATOM | 4958 | CB | ASN | B | 478 | −10.211 | 7.874 | 86.190 | 1.00 | 33.86 | C |
| ATOM | 4959 | CG | ASN | B | 478 | −11.044 | 9.049 | 86.648 | 1.00 | 34.09 | C |
| ATOM | 4960 | OD1 | ASN | B | 478 | −11.688 | 9.716 | 85.838 | 1.00 | 34.86 | O |
| ATOM | 4961 | ND2 | ASN | B | 478 | −11.020 | 9.325 | 87.948 | 1.00 | 33.98 | N |
| ATOM | 4962 | N | GLY | B | 479 | −6.713 | 7.399 | 85.384 | 1.00 | 34.33 | N |
| ATOM | 4963 | CA | GLY | B | 479 | −5.787 | 6.540 | 84.655 | 1.00 | 34.50 | C |
| ATOM | 4964 | C | GLY | B | 479 | −6.074 | 6.444 | 83.170 | 1.00 | 34.69 | C |
| ATOM | 4965 | O | GLY | B | 479 | −5.643 | 5.495 | 82.522 | 1.00 | 34.83 | O |
| ATOM | 4966 | N | THR | B | 480 | −6.787 | 7.428 | 82.627 | 1.00 | 34.83 | N |
| ATOM | 4967 | CA | THR | B | 480 | −7.160 | 7.425 | 81.210 | 1.00 | 35.00 | C |
| ATOM | 4968 | C | THR | B | 480 | −5.990 | 7.825 | 80.325 | 1.00 | 35.11 | C |
| ATOM | 4969 | O | THR | B | 480 | −5.391 | 8.889 | 80.509 | 1.00 | 35.27 | O |
| ATOM | 4970 | CB | THR | B | 480 | −8.376 | 8.343 | 80.923 | 1.00 | 35.01 | C |
| ATOM | 4971 | OG1 | THR | B | 480 | −9.484 | 7.936 | 81.731 | 1.00 | 35.17 | O |
| ATOM | 4972 | CG2 | THR | B | 480 | −8.785 | 8.274 | 79.450 | 1.00 | 34.87 | C |
| ATOM | 4973 | N | VAL | B | 481 | −5.676 | 6.968 | 79.363 | 1.00 | 35.21 | N |
| ATOM | 4974 | CA | VAL | B | 481 | −4.599 | 7.241 | 78.431 | 1.00 | 35.47 | C |
| ATOM | 4975 | C | VAL | B | 481 | −5.064 | 8.246 | 77.390 | 1.00 | 36.09 | C |
| ATOM | 4976 | O | VAL | B | 481 | −6.089 | 8.052 | 76.730 | 1.00 | 36.25 | O |
| ATOM | 4977 | CB | VAL | B | 481 | −4.075 | 5.960 | 77.747 | 1.00 | 35.23 | C |
| ATOM | 4978 | CG1 | VAL | B | 481 | −2.929 | 6.289 | 76.803 | 1.00 | 34.55 | C |
| ATOM | 4979 | CG2 | VAL | B | 481 | −3.635 | 4.939 | 78.792 | 1.00 | 34.62 | C |
| ATOM | 4980 | N | TYR | B | 482 | −4.303 | 9.326 | 77.267 | 1.00 | 36.59 | N |
| ATOM | 4981 | CA | TYR | B | 482 | −4.553 | 10.342 | 76.265 | 1.00 | 37.10 | C |
| ATOM | 4982 | C | TYR | B | 482 | −3.512 | 10.260 | 75.165 | 1.00 | 37.54 | C |
| ATOM | 4983 | O | TYR | B | 482 | −2.351 | 9.952 | 75.415 | 1.00 | 37.60 | O |
| ATOM | 4984 | CB | TYR | B | 482 | −4.548 | 11.731 | 76.905 | 1.00 | 37.08 | C |
| ATOM | 4985 | CG | TYR | B | 482 | −5.889 | 12.163 | 77.461 | 1.00 | 36.99 | C |
| ATOM | 4986 | CD1 | TYR | B | 482 | −6.545 | 13.282 | 76.946 | 1.00 | 36.98 | C |
| ATOM | 4987 | CD2 | TYR | B | 482 | −6.504 | 11.457 | 78.496 | 1.00 | 36.85 | C |
| ATOM | 4988 | CE1 | TYR | B | 482 | −7.777 | 13.692 | 77.444 | 1.00 | 36.44 | C |
| ATOM | 4989 | CE2 | TYR | B | 482 | −7.734 | 11.855 | 79.001 | 1.00 | 37.41 | C |
| ATOM | 4990 | CZ | TYR | B | 482 | −8.366 | 12.975 | 78.467 | 1.00 | 37.38 | C |
| ATOM | 4991 | OH | TYR | B | 482 | −9.586 | 13.378 | 78.962 | 1.00 | 37.33 | O |
| ATOM | 4992 | N | THR | B | 483 | −3.953 | 10.538 | 73.946 | 1.00 | 38.19 | N |
| ATOM | 4993 | CA | THR | B | 483 | −3.114 | 10.537 | 72.758 | 1.00 | 38.71 | C |
| ATOM | 4994 | C | THR | B | 483 | −2.107 | 11.707 | 72.848 | 1.00 | 39.18 | C |
| ATOM | 4995 | O | THR | B | 483 | −2.321 | 12.634 | 73.637 | 1.00 | 39.19 | O |
| ATOM | 4996 | CB | THR | B | 483 | −4.034 | 10.626 | 71.500 | 1.00 | 38.72 | C |
| ATOM | 4997 | OG1 | THR | B | 483 | −3.432 | 9.973 | 70.377 | 1.00 | 39.19 | O |
| ATOM | 4998 | CG2 | THR | B | 483 | −4.348 | 12.051 | 71.144 | 1.00 | 38.78 | C |
| ATOM | 4999 | N | PRO | B | 484 | −0.986 | 11.650 | 72.090 | 1.00 | 39.66 | N |
| ATOM | 5000 | CA | PRO | B | 484 | −0.061 | 12.794 | 72.031 | 1.00 | 40.06 | C |
| ATOM | 5001 | C | PRO | B | 484 | −0.734 | 14.132 | 71.686 | 1.00 | 40.75 | C |
| ATOM | 5002 | O | PRO | B | 484 | −0.290 | 15.175 | 72.165 | 1.00 | 40.76 | O |
| ATOM | 5003 | CB | PRO | B | 484 | 0.926 | 12.394 | 70.925 | 1.00 | 39.87 | C |
| ATOM | 5004 | CG | PRO | B | 484 | 0.928 | 10.927 | 70.934 | 1.00 | 39.54 | C |
| ATOM | 5005 | CD | PRO | B | 484 | −0.480 | 10.511 | 71.296 | 1.00 | 39.65 | C |
| ATOM | 5006 | N | ASP | B | 485 | −1.790 | 14.090 | 70.870 | 1.00 | 41.57 | N |
| ATOM | 5007 | CA | ASP | B | 485 | −2.595 | 15.273 | 70.520 | 1.00 | 42.20 | C |
| ATOM | 5008 | C | ASP | B | 485 | −3.411 | 15.852 | 71.671 | 1.00 | 42.48 | C |
| ATOM | 5009 | O | ASP | B | 485 | −3.785 | 17.022 | 71.638 | 1.00 | 42.57 | O |
| ATOM | 5010 | CB | ASP | B | 485 | −3.560 | 14.937 | 69.381 | 1.00 | 42.35 | C |
| ATOM | 5011 | CG | ASP | B | 485 | −2.941 | 15.112 | 68.021 | 1.00 | 43.06 | C |
| ATOM | 5012 | OD1 | ASP | B | 485 | −1.752 | 15.487 | 67.950 | 1.00 | 44.40 | O |
| ATOM | 5013 | OD2 | ASP | B | 485 | −3.651 | 14.882 | 67.019 | 1.00 | 44.07 | O |
| ATOM | 5014 | N | GLY | B | 486 | −3.708 | 15.027 | 72.668 | 1.00 | 43.00 | N |
| ATOM | 5015 | CA | GLY | B | 486 | −4.550 | 15.437 | 73.787 | 1.00 | 43.79 | C |
| ATOM | 5016 | C | GLY | B | 486 | −5.975 | 14.921 | 73.680 | 1.00 | 44.40 | C |
| ATOM | 5017 | O | GLY | B | 486 | −6.887 | 15.449 | 74.321 | 1.00 | 44.60 | O |
| ATOM | 5018 | N | LYS | B | 487 | −6.168 | 13.890 | 72.866 | 1.00 | 44.94 | N |
| ATOM | 5019 | CA | LYS | B | 487 | −7.469 | 13.246 | 72.723 | 1.00 | 45.58 | C |
| ATOM | 5020 | C | LYS | B | 487 | −7.536 | 11.987 | 73.576 | 1.00 | 45.76 | C |
| ATOM | 5021 | O | LYS | B | 487 | −6.605 | 11.177 | 73.581 | 1.00 | 45.79 | O |
| ATOM | 5022 | CB | LYS | B | 487 | −7.766 | 12.916 | 71.251 | 1.00 | 45.55 | C |
| ATOM | 5023 | CG | LYS | B | 487 | −8.121 | 14.131 | 70.373 | 1.00 | 46.34 | C |
| ATOM | 5024 | CD | LYS | B | 487 | −9.510 | 14.703 | 70.706 | 1.00 | 47.61 | C |
| ATOM | 5025 | CE | LYS | B | 487 | −10.064 | 15.580 | 69.580 | 1.00 | 48.62 | C |
| ATOM | 5026 | NZ | LYS | B | 487 | −9.302 | 16.854 | 69.396 | 1.00 | 48.99 | N |
| ATOM | 5027 | N | ALA | B | 488 | −8.638 | 11.839 | 74.304 | 1.00 | 46.09 | N |
| ATOM | 5028 | CA | ALA | B | 488 | −8.883 | 10.652 | 75.115 | 1.00 | 46.50 | C |
| ATOM | 5029 | C | ALA | B | 488 | −9.003 | 9.401 | 74.244 | 1.00 | 46.82 | C |
| ATOM | 5030 | O | ALA | B | 488 | −9.837 | 9.347 | 73.338 | 1.00 | 46.85 | O |
| ATOM | 5031 | CB | ALA | B | 488 | −10.143 | 10.841 | 75.956 | 1.00 | 46.32 | C |
| ATOM | 5032 | N | THR | B | 489 | −8.151 | 8.409 | 74.504 | 1.00 | 47.40 | N |
| ATOM | 5033 | CA | THR | B | 489 | −8.300 | 7.093 | 73.876 | 1.00 | 47.81 | C |
| ATOM | 5034 | C | THR | B | 489 | −9.437 | 6.372 | 74.575 | 1.00 | 48.27 | C |
| ATOM | 5035 | O | THR | B | 489 | −9.983 | 5.402 | 74.042 | 1.00 | 48.37 | O |
| ATOM | 5036 | CB | THR | B | 489 | −7.020 | 6.214 | 73.948 | 1.00 | 47.75 | C |
| ATOM | 5037 | OG1 | THR | B | 489 | −6.508 | 6.198 | 75.287 | 1.00 | 47.54 | O |
| ATOM | 5038 | CG2 | THR | B | 489 | −5.951 | 6.720 | 72.990 | 1.00 | 47.59 | C |
| ATOM | 5039 | N | ASP | B | 490 | −9.767 | 6.866 | 75.776 | 1.00 | 48.73 | N |
| ATOM | 5040 | CA | ASP | B | 490 | −10.922 | 6.442 | 76.585 | 1.00 | 49.20 | C |
| ATOM | 5041 | C | ASP | B | 490 | −10.882 | 4.945 | 76.883 | 1.00 | 49.13 | C |
| ATOM | 5042 | O | ASP | B | 490 | −10.995 | 4.518 | 78.046 | 1.00 | 49.38 | O |
| ATOM | 5043 | CB | ASP | B | 490 | −12.252 | 6.844 | 75.905 | 1.00 | 49.54 | C |
| ATOM | 5044 | CG | ASP | B | 490 | −13.424 | 6.953 | 76.887 | 1.00 | 50.32 | C |
| ATOM | 5045 | OD1 | ASP | B | 490 | −13.214 | 6.819 | 78.115 | 1.00 | 51.06 | O |
| ATOM | 5046 | OD2 | ASP | B | 490 | −14.566 | 7.183 | 76.422 | 1.00 | 51.02 | O |
| ATOM | 5047 | N | TYR | B | 491 | −10.715 | 4.168 | 75.813 | 1.00 | 48.63 | N |
| ATOM | 5048 | CA | TYR | B | 491 | −10.610 | 2.723 | 75.871 | 1.00 | 48.08 | C |
| ATOM | 5049 | C | TYR | B | 491 | −9.418 | 2.354 | 76.738 | 1.00 | 47.37 | C |
| ATOM | 5050 | O | TYR | B | 491 | −9.563 | 1.624 | 77.724 | 1.00 | 47.37 | O |
| ATOM | 5051 | CB | TYR | B | 491 | −10.479 | 2.162 | 74.450 | 1.00 | 48.26 | C |
| ATOM | 5052 | CG | TYR | B | 491 | −11.749 | 2.271 | 73.620 | 1.00 | 48.60 | C |
| ATOM | 5053 | CD1 | TYR | B | 491 | −12.742 | 3.212 | 73.925 | 1.00 | 48.60 | C |
| ATOM | 5054 | CD2 | TYR | B | 491 | −11.949 | 1.444 | 72.513 | 1.00 | 49.51 | C |
| ATOM | 5055 | CE1 | TYR | B | 491 | −13.908 | 3.310 | 73.163 | 1.00 | 49.14 | C |
| ATOM | 5056 | CE2 | TYR | B | 491 | −13.112 | 1.535 | 71.739 | 1.00 | 49.53 | C |
| ATOM | 5057 | CZ | TYR | B | 491 | −14.085 | 2.469 | 72.070 | 1.00 | 49.17 | C |
| ATOM | 5058 | OH | TYR | B | 491 | −15.229 | 2.556 | 71.309 | 1.00 | 48.98 | O |
| ATOM | 5059 | N | ARG | B | 492 | −8.255 | 2.905 | 76.397 | 1.00 | 46.47 | N |
| ATOM | 5060 | CA | ARG | B | 492 | −7.036 | 2.648 | 77.153 | 1.00 | 45.52 | C |
| ATOM | 5061 | C | ARG | B | 492 | −6.982 | 3.389 | 78.485 | 1.00 | 45.00 | C |
| ATOM | 5062 | O | ARG | B | 492 | −7.197 | 4.599 | 78.550 | 1.00 | 44.71 | O |
| ATOM | 5063 | CB | ARG | B | 492 | −5.797 | 2.927 | 76.309 | 1.00 | 45.40 | C |
| ATOM | 5064 | CG | ARG | B | 492 | −5.265 | 1.692 | 75.632 | 1.00 | 44.99 | C |
| ATOM | 5065 | CD | ARG | B | 492 | −5.715 | 1.564 | 74.197 | 1.00 | 44.28 | C |
| ATOM | 5066 | NE | ARG | B | 492 | −5.606 | 0.171 | 73.782 | 1.00 | 45.00 | N |
| ATOM | 5067 | CZ | ARG | B | 492 | −5.443 | −0.246 | 72.530 | 1.00 | 45.37 | C |
| ATOM | 5068 | NH1 | ARG | B | 492 | −5.345 | 0.620 | 71.531 | 1.00 | 45.41 | N |
| ATOM | 5069 | NH2 | ARG | B | 492 | −5.362 | −1.545 | 72.280 | 1.00 | 45.76 | N |
| ATOM | 5070 | N | VAL | B | 493 | −6.703 | 2.627 | 79.538 | 1.00 | 44.56 | N |
| ATOM | 5071 | CA | VAL | B | 493 | −6.642 | 3.117 | 80.916 | 1.00 | 44.28 | C |
| ATOM | 5072 | C | VAL | B | 493 | −5.642 | 2.287 | 81.723 | 1.00 | 43.90 | C |
| ATOM | 5073 | O | VAL | B | 493 | −5.492 | 1.089 | 81.477 | 1.00 | 43.82 | O |
| ATOM | 5074 | CB | VAL | B | 493 | −8.026 | 3.042 | 81.620 | 1.00 | 44.24 | C |
| ATOM | 5075 | CG1 | VAL | B | 493 | −8.808 | 4.328 | 81.429 | 1.00 | 44.52 | C |
| ATOM | 5076 | CG2 | VAL | B | 493 | −8.829 | 1.844 | 81.124 | 1.00 | 44.55 | C |
| ATOM | 5077 | N | VAL | B | 494 | −4.957 | 2.918 | 82.678 | 1.00 | 43.67 | N |
| ATOM | 5078 | CA | VAL | B | 494 | −4.056 | 2.185 | 83.578 | 1.00 | 43.57 | C |
| ATOM | 5079 | C | VAL | B | 494 | −4.565 | 2.189 | 85.022 | 1.00 | 43.68 | C |
| ATOM | 5080 | O | VAL | B | 494 | −5.230 | 3.133 | 85.455 | 1.00 | 43.48 | O |
| ATOM | 5081 | CB | VAL | B | 494 | −2.558 | 2.655 | 83.498 | 1.00 | 43.53 | C |
| ATOM | 5082 | CG1 | VAL | B | 494 | −2.168 | 3.049 | 82.077 | 1.00 | 43.20 | C |
| ATOM | 5083 | CG2 | VAL | B | 494 | −2.273 | 3.794 | 84.452 | 1.00 | 43.38 | C |
| ATOM | 5084 | N | VAL | B | 495 | −4.254 | 1.122 | 85.753 | 1.00 | 44.03 | N |
| ATOM | 5085 | CA | VAL | B | 495 | −4.661 | 1.001 | 87.150 | 1.00 | 44.48 | C |
| ATOM | 5086 | C | VAL | B | 495 | −3.804 | 1.910 | 88.032 | 1.00 | 44.90 | C |
| ATOM | 5087 | O | VAL | B | 495 | −2.607 | 1.680 | 88.185 | 1.00 | 44.78 | O |
| ATOM | 5088 | CB | VAL | B | 495 | −4.597 | −0.472 | 87.646 | 1.00 | 44.37 | C |
| ATOM | 5089 | CG1 | VAL | B | 495 | −5.027 | −0.574 | 89.095 | 1.00 | 44.07 | C |
| ATOM | 5090 | CG2 | VAL | B | 495 | −5.472 | −1.362 | 86.785 | 1.00 | 44.22 | C |
| ATOM | 5091 | N | ASP | B | 496 | −4.434 | 2.941 | 88.596 | 1.00 | 45.73 | N |
| ATOM | 5092 | CA | ASP | B | 496 | −3.768 | 3.910 | 89.481 | 1.00 | 46.53 | C |
| ATOM | 5093 | C | ASP | B | 496 | −4.178 | 3.782 | 90.951 | 1.00 | 46.88 | C |
| ATOM | 5094 | O | ASP | B | 496 | −3.304 | 3.632 | 91.809 | 1.00 | 47.07 | O |
| ATOM | 5095 | CB | ASP | B | 496 | −3.970 | 5.346 | 88.983 | 1.00 | 46.64 | C |
| ATOM | 5096 | CG | ASP | B | 496 | −3.167 | 5.644 | 87.744 | 1.00 | 47.45 | C |
| ATOM | 5097 | OD1 | ASP | B | 496 | −2.327 | 4.798 | 87.374 | 1.00 | 48.23 | O |
| ATOM | 5098 | OD2 | ASP | B | 496 | −3.366 | 6.722 | 87.143 | 1.00 | 49.08 | O |
| ATOM | 5099 | N | PRO | B | 497 | −5.493 | 3.893 | 91.260 | 1.00 | 47.23 | N |
| ATOM | 5100 | CA | PRO | B | 497 | −5.940 | 3.378 | 92.550 | 1.00 | 47.34 | C |
| ATOM | 5101 | C | PRO | B | 497 | −5.970 | 1.847 | 92.491 | 1.00 | 47.47 | C |
| ATOM | 5102 | O | PRO | B | 497 | −6.923 | 1.244 | 91.990 | 1.00 | 47.57 | O |
| ATOM | 5103 | CB | PRO | B | 497 | −7.350 | 3.969 | 92.706 | 1.00 | 47.34 | C |
| ATOM | 5104 | CG | PRO | B | 497 | −7.475 | 5.021 | 91.634 | 1.00 | 47.36 | C |
| ATOM | 5105 | CD | PRO | B | 497 | −6.606 | 4.524 | 90.527 | 1.00 | 47.35 | C |
| ATOM | 5106 | N | VAL | B | 498 | −4.905 | 1.241 | 92.998 | 1.00 | 47.54 | N |
| ATOM | 5107 | CA | VAL | B | 498 | −4.650 | −0.187 | 92.852 | 1.00 | 47.57 | C |
| ATOM | 5108 | C | VAL | B | 498 | −5.088 | −0.965 | 94.114 | 1.00 | 47.58 | C |
| ATOM | 5109 | O | VAL | B | 498 | −5.123 | −0.389 | 95.204 | 1.00 | 47.55 | O |
| ATOM | 5110 | CB | VAL | B | 498 | −3.155 | −0.399 | 92.454 | 1.00 | 47.54 | C |
| ATOM | 5111 | CG1 | VAL | B | 498 | −2.248 | 0.603 | 93.163 | 1.00 | 47.53 | C |
| ATOM | 5112 | CG2 | VAL | B | 498 | −2.700 | −1.797 | 92.708 | 1.00 | 47.71 | C |
| ATOM | 5113 | N | LYS | B | 499 | −5.420 | −2.257 | 93.964 | 1.00 | 47.62 | N |
| ATOM | 5114 | CA | LYS | B | 499 | −6.095 | −3.032 | 95.038 | 1.00 | 47.52 | C |
| ATOM | 5115 | C | LYS | B | 499 | −5.316 | −4.166 | 95.779 | 1.00 | 47.55 | C |
| ATOM | 5116 | O | LYS | B | 499 | −4.892 | −3.944 | 96.920 | 1.00 | 47.69 | O |
| ATOM | 5117 | CB | LYS | B | 499 | −7.483 | −3.501 | 94.573 | 1.00 | 47.66 | C |
| ATOM | 5118 | CG | LYS | B | 499 | −8.482 | −2.372 | 94.355 | 1.00 | 47.83 | C |
| ATOM | 5119 | CD | LYS | B | 499 | −9.471 | −2.721 | 93.249 | 1.00 | 48.58 | C |
| ATOM | 5120 | CE | LYS | B | 499 | −10.754 | −1.884 | 93.342 | 1.00 | 49.19 | C |
| ATOM | 5121 | NZ | LYS | B | 499 | −11.735 | −2.426 | 94.339 | 1.00 | 48.55 | N |
| ATOM | 5122 | N | PRO | B | 500 | −5.153 | −5.372 | 95.161 | 1.00 | 47.27 | N |
| ATOM | 5123 | CA | PRO | B | 500 | −4.558 | −6.596 | 95.774 | 1.00 | 47.10 | C |
| ATOM | 5124 | C | PRO | B | 500 | −3.345 | −6.420 | 96.718 | 1.00 | 46.77 | C |
| ATOM | 5125 | O | PRO | B | 500 | −3.506 | −5.952 | 97.844 | 1.00 | 46.86 | O |
| ATOM | 5126 | CB | PRO | B | 500 | −4.168 | −7.433 | 94.553 | 1.00 | 47.07 | C |
| ATOM | 5127 | CG | PRO | B | 500 | −5.173 | −7.076 | 93.525 | 1.00 | 46.97 | C |
| ATOM | 5128 | CD | PRO | B | 500 | −5.583 | −5.642 | 93.773 | 1.00 | 47.31 | C |
| ATOM | 5129 | N | ALA | B | 501 | −2.159 | −6.845 | 96.279 | 1.00 | 46.39 | N |
| ATOM | 5130 | CA | ALA | B | 501 | −0.905 | −6.513 | 96.962 | 1.00 | 45.85 | C |
| ATOM | 5131 | C | ALA | B | 501 | −0.286 | −5.293 | 96.265 | 1.00 | 45.58 | C |
| ATOM | 5132 | O | ALA | B | 501 | 0.920 | −5.022 | 96.370 | 1.00 | 45.52 | O |
| ATOM | 5133 | CB | ALA | B | 501 | 0.041 | −7.689 | 96.929 | 1.00 | 45.82 | C |
| ATOM | 5134 | N | TYR | B | 502 | −1.153 | −4.557 | 95.569 | 1.00 | 44.97 | N |
| ATOM | 5135 | CA | TYR | B | 502 | −0.776 | −3.530 | 94.600 | 1.00 | 44.29 | C |
| ATOM | 5136 | C | TYR | B | 502 | −0.061 | −4.138 | 93.394 | 1.00 | 43.76 | C |
| ATOM | 5137 | O | TYR | B | 502 | 0.512 | −3.422 | 92.570 | 1.00 | 43.85 | O |
| ATOM | 5138 | CB | TYR | B | 502 | 0.013 | −2.385 | 95.254 | 1.00 | 44.45 | C |
| ATOM | 5139 | CG | TYR | B | 502 | −0.705 | −1.786 | 96.444 | 1.00 | 44.56 | C |
| ATOM | 5140 | CD1 | TYR | B | 502 | −2.002 | −1.287 | 96.319 | 1.00 | 44.68 | C |
| ATOM | 5141 | CD2 | TYR | B | 502 | −0.096 | −1.729 | 97.695 | 1.00 | 44.75 | C |
| ATOM | 5142 | CE1 | TYR | B | 502 | −2.673 | −0.751 | 97.403 | 1.00 | 44.48 | C |
| ATOM | 5143 | CE2 | TYR | B | 502 | −0.761 | −1.188 | 98.789 | 1.00 | 44.98 | C |
| ATOM | 5144 | CZ | TYR | B | 502 | −2.049 | −0.704 | 98.632 | 1.00 | 44.49 | C |
| ATOM | 5145 | OH | TYR | B | 502 | −2.716 | −0.159 | 99.701 | 1.00 | 44.77 | O |
| ATOM | 5146 | N | SER | B | 503 | −0.144 | −5.463 | 93.286 | 1.00 | 42.89 | N |
| ATOM | 5147 | CA | SER | B | 503 | 0.466 | −6.222 | 92.198 | 1.00 | 42.11 | C |
| ATOM | 5148 | C | SER | B | 503 | −0.153 | −5.900 | 90.841 | 1.00 | 41.49 | C |
| ATOM | 5149 | O | SER | B | 503 | 0.395 | −6.271 | 89.804 | 1.00 | 41.54 | O |
| ATOM | 5150 | CB | SER | B | 503 | 0.338 | −7.724 | 92.467 | 1.00 | 42.22 | C |
| ATOM | 5151 | OG | SER | B | 503 | −1.015 | −8.144 | 92.366 | 1.00 | 42.13 | O |
| ATOM | 5152 | N | ASP | B | 504 | −1.298 | −5.224 | 90.857 | 1.00 | 40.60 | N |
| ATOM | 5153 | CA | ASP | B | 504 | −2.017 | −4.868 | 89.635 | 1.00 | 39.83 | C |
| ATOM | 5154 | C | ASP | B | 504 | −1.732 | −3.430 | 89.195 | 1.00 | 39.14 | C |
| ATOM | 5155 | O | ASP | B | 504 | −2.245 | −2.976 | 88.174 | 1.00 | 38.99 | O |
| ATOM | 5156 | CB | ASP | B | 504 | −3.526 | −5.079 | 89.820 | 1.00 | 39.83 | C |
| ATOM | 5157 | CG | ASP | B | 504 | −4.075 | −4.319 | 91.009 | 1.00 | 40.22 | C |
| ATOM | 5158 | OD1 | ASP | B | 504 | −3.477 | −4.418 | 92.103 | 1.00 | 39.93 | O |
| ATOM | 5159 | OD2 | ASP | B | 504 | −5.095 | −3.613 | 90.856 | 1.00 | 41.05 | O |
| ATOM | 5160 | N | LYS | B | 505 | −0.917 | −2.714 | 89.966 | 1.00 | 38.46 | N |
| ATOM | 5161 | CA | LYS | B | 505 | −0.542 | −1.352 | 89.603 | 1.00 | 37.83 | C |
| ATOM | 5162 | C | LYS | B | 505 | 0.139 | −1.349 | 88.244 | 1.00 | 37.57 | C |
| ATOM | 5163 | O | LYS | B | 505 | 1.052 | −2.132 | 87.997 | 1.00 | 37.74 | O |
| ATOM | 5164 | CB | LYS | B | 505 | 0.372 | −0.722 | 90.657 | 1.00 | 37.67 | C |
| ATOM | 5165 | CG | LYS | B | 505 | 0.691 | 0.737 | 90.377 | 1.00 | 36.47 | C |
| ATOM | 5166 | CD | LYS | B | 505 | 1.435 | 1.410 | 91.509 | 1.00 | 34.02 | C |
| ATOM | 5167 | CE | LYS | B | 505 | 1.928 | 2.783 | 91.073 | 1.00 | 33.33 | C |
| ATOM | 5168 | NZ | LYS | B | 505 | 0.834 | 3.696 | 90.620 | 1.00 | 31.67 | N |
| ATOM | 5169 | N | GLY | B | 506 | −0.330 | −0.477 | 87.363 | 1.00 | 37.32 | N |
| ATOM | 5170 | CA | GLY | B | 506 | 0.247 | −0.348 | 86.033 | 1.00 | 36.98 | C |
| ATOM | 5171 | C | GLY | B | 506 | −0.454 | −1.153 | 84.957 | 1.00 | 36.68 | C |
| ATOM | 5172 | O | GLY | B | 506 | −0.210 | −0.935 | 83.771 | 1.00 | 36.72 | O |
| ATOM | 5173 | N | ASP | B | 507 | −1.315 | −2.085 | 85.367 | 1.00 | 36.40 | N |
| ATOM | 5174 | CA | ASP | B | 507 | −2.087 | −2.897 | 84.426 | 1.00 | 36.06 | C |
| ATOM | 5175 | C | ASP | B | 507 | −2.811 | −2.019 | 83.418 | 1.00 | 35.94 | C |
| ATOM | 5176 | O | ASP | B | 507 | −3.419 | −1.006 | 83.782 | 1.00 | 35.70 | O |
| ATOM | 5177 | CB | ASP | B | 507 | −3.103 | −3.772 | 85.163 | 1.00 | 36.09 | C |
| ATOM | 5178 | CG | ASP | B | 507 | −2.469 | −4.961 | 85.849 | 1.00 | 35.83 | C |
| ATOM | 5179 | OD1 | ASP | B | 507 | −1.249 | −5.170 | 85.690 | 1.00 | 36.28 | O |
| ATOM | 5180 | OD2 | ASP | B | 507 | −3.197 | −5.691 | 86.553 | 1.00 | 35.77 | O |
| ATOM | 5181 | N | LEU | B | 508 | −2.729 | −2.408 | 82.152 | 1.00 | 35.89 | N |
| ATOM | 5182 | CA | LEU | B | 508 | −3.341 | −1.637 | 81.081 | 1.00 | 36.05 | C |
| ATOM | 5183 | C | LEU | B | 508 | −4.572 | −2.342 | 80.537 | 1.00 | 35.98 | C |
| ATOM | 5184 | O | LEU | B | 508 | −4.528 | −3.526 | 80.205 | 1.00 | 35.84 | O |
| ATOM | 5185 | CB | LEU | B | 508 | −2.349 | −1.370 | 79.944 | 1.00 | 35.91 | C |
| ATOM | 5186 | CG | LEU | B | 508 | −2.902 | −0.335 | 78.961 | 1.00 | 36.35 | C |
| ATOM | 5187 | CD1 | LEU | B | 508 | −2.059 | 0.936 | 78.962 | 1.00 | 36.19 | C |
| ATOM | 5188 | CD2 | LEU | B | 508 | −3.013 | −0.916 | 77.563 | 1.00 | 36.59 | C |
| ATOM | 5189 | N | TYR | B | 509 | −5.662 | −1.590 | 80.437 | 1.00 | 36.29 | N |
| ATOM | 5190 | CA | TYR | B | 509 | −6.930 | −2.112 | 79.953 | 1.00 | 36.64 | C |
| ATOM | 5191 | C | TYR | B | 509 | −7.396 | −1.409 | 78.682 | 1.00 | 37.03 | C |
| ATOM | 5192 | O | TYR | B | 509 | −7.085 | −0.238 | 78.451 | 1.00 | 37.00 | O |
| ATOM | 5193 | CB | TYR | B | 509 | −8.008 | −1.964 | 81.035 | 1.00 | 36.59 | C |
| ATOM | 5194 | CG | TYR | B | 509 | −7.907 | −2.957 | 82.175 | 1.00 | 36.39 | C |
| ATOM | 5195 | CD1 | TYR | B | 509 | −6.904 | −2.843 | 83.139 | 1.00 | 36.59 | C |
| ATOM | 5196 | CD2 | TYR | B | 509 | −8.825 | −4.000 | 82.299 | 1.00 | 36.16 | C |
| ATOM | 5197 | CE1 | TYR | B | 509 | −6.804 | −3.748 | 84.189 | 1.00 | 36.89 | C |
| ATOM | 5198 | CE2 | TYR | B | 509 | −8.739 | −4.916 | 83.349 | 1.00 | 36.25 | C |
| ATOM | 5199 | CZ | TYR | B | 509 | −7.724 | −4.783 | 84.290 | 1.00 | 36.97 | C |
| ATOM | 5200 | OH | TYR | B | 509 | −7.618 | −5.677 | 85.336 | 1.00 | 36.84 | O |
| ATOM | 5201 | N | LYS | B | 510 | −8.137 | −2.143 | 77.858 | 1.00 | 37.53 | N |
| ATOM | 5202 | CA | LYS | B | 510 | −8.964 | −1.538 | 76.822 | 1.00 | 37.99 | C |
| ATOM | 5203 | C | LYS | B | 510 | −10.426 | −1.799 | 77.205 | 1.00 | 38.18 | C |
| ATOM | 5204 | O | LYS | B | 510 | −11.029 | −2.784 | 76.771 | 1.00 | 38.33 | O |
| ATOM | 5205 | CB | LYS | B | 510 | −8.638 | −2.116 | 75.445 | 1.00 | 37.95 | C |
| ATOM | 5206 | CG | LYS | B | 510 | −8.669 | −1.072 | 74.351 | 1.00 | 38.45 | C |
| ATOM | 5207 | CD | LYS | B | 510 | −9.262 | −1.598 | 73.064 | 1.00 | 38.67 | C |
| ATOM | 5208 | CE | LYS | B | 510 | −9.167 | −0.543 | 71.979 | 1.00 | 39.11 | C |
| ATOM | 5209 | NZ | LYS | B | 510 | −10.094 | −0.802 | 70.847 | 1.00 | 39.27 | N |
| ATOM | 5210 | N | GLY | B | 511 | −10.978 | −0.917 | 78.037 | 1.00 | 38.46 | N |
| ATOM | 5211 | CA | GLY | B | 511 | −12.291 | −1.130 | 78.650 | 1.00 | 38.62 | C |
| ATOM | 5212 | C | GLY | B | 511 | −12.153 | −2.042 | 79.858 | 1.00 | 38.90 | C |
| ATOM | 5213 | O | GLY | B | 511 | −11.447 | −1.711 | 80.815 | 1.00 | 39.14 | O |
| ATOM | 5214 | N | ASN | B | 512 | −12.827 | −3.190 | 79.821 | 1.00 | 38.83 | N |
| ATOM | 5215 | CA | ASN | B | 512 | −12.661 | −4.215 | 80.859 | 1.00 | 38.75 | C |
| ATOM | 5216 | C | ASN | B | 512 | −11.716 | −5.337 | 80.423 | 1.00 | 38.24 | C |
| ATOM | 5217 | O | ASN | B | 512 | −11.568 | −6.348 | 81.118 | 1.00 | 38.28 | O |
| ATOM | 5218 | CB | ASN | B | 512 | −14.017 | −4.781 | 81.310 | 1.00 | 38.92 | C |
| ATOM | 5219 | CG | ASN | B | 512 | −14.533 | −4.122 | 82.585 | 1.00 | 39.96 | C |
| ATOM | 5220 | OD1 | ASN | B | 512 | −13.947 | −4.269 | 83.664 | 1.00 | 41.02 | O |
| ATOM | 5221 | ND2 | ASN | B | 512 | −15.638 | −3.397 | 82.465 | 1.00 | 40.62 | N |
| ATOM | 5222 | N | GLN | B | 513 | −11.078 | −5.132 | 79.272 | 1.00 | 37.51 | N |
| ATOM | 5223 | CA | GLN | B | 513 | −10.123 | −6.075 | 78.705 | 1.00 | 36.86 | C |
| ATOM | 5224 | C | GLN | B | 513 | −8.694 | −5.773 | 79.183 | 1.00 | 36.53 | C |
| ATOM | 5225 | O | GLN | B | 513 | −8.072 | −4.810 | 78.734 | 1.00 | 36.53 | O |
| ATOM | 5226 | CB | GLN | B | 513 | −10.209 | −6.028 | 77.170 | 1.00 | 36.66 | C |
| ATOM | 5227 | CG | GLN | B | 513 | −9.279 | −6.984 | 76.436 | 1.00 | 36.53 | C |
| ATOM | 5228 | CD | GLN | B | 513 | −9.660 | −8.435 | 76.634 | 1.00 | 36.49 | C |
| ATOM | 5229 | OE1 | GLN | B | 513 | −10.741 | −8.867 | 76.229 | 1.00 | 37.23 | O |
| ATOM | 5230 | NE2 | GLN | B | 513 | −8.773 | −9.198 | 77.260 | 1.00 | 35.70 | N |
| ATOM | 5231 | N | LEU | B | 514 | −8.188 | −6.602 | 80.094 | 1.00 | 36.17 | N |
| ATOM | 5232 | CA | LEU | B | 514 | −6.810 | −6.510 | 80.578 | 1.00 | 35.94 | C |
| ATOM | 5233 | C | LEU | B | 514 | −5.837 | −6.947 | 79.486 | 1.00 | 35.78 | C |
| ATOM | 5234 | O | LEU | B | 514 | −5.864 | −8.098 | 79.059 | 1.00 | 35.97 | O |
| ATOM | 5235 | CB | LEU | B | 514 | −6.627 | −7.381 | 81.830 | 1.00 | 35.98 | C |
| ATOM | 5236 | CG | LEU | B | 514 | −5.207 | −7.757 | 82.282 | 1.00 | 36.05 | C |
| ATOM | 5237 | CD1 | LEU | B | 514 | −4.488 | −6.576 | 82.930 | 1.00 | 36.24 | C |
| ATOM | 5238 | CD2 | LEU | B | 514 | −5.251 | −8.940 | 83.234 | 1.00 | 35.94 | C |
| ATOM | 5239 | N | LEU | B | 515 | −4.977 | −6.032 | 79.046 | 1.00 | 35.51 | N |
| ATOM | 5240 | CA | LEU | B | 515 | −4.056 | −6.305 | 77.934 | 1.00 | 34.98 | C |
| ATOM | 5241 | C | LEU | B | 515 | −2.632 | −6.640 | 78.370 | 1.00 | 34.55 | C |
| ATOM | 5242 | O | LEU | B | 515 | −1.983 | −7.501 | 77.777 | 1.00 | 34.62 | O |
| ATOM | 5243 | CB | LEU | B | 515 | −4.028 | −5.131 | 76.952 | 1.00 | 35.04 | C |
| ATOM | 5244 | CG | LEU | B | 515 | −5.260 | −4.831 | 76.093 | 1.00 | 34.97 | C |
| ATOM | 5245 | CD1 | LEU | B | 515 | −5.085 | −3.481 | 75.415 | 1.00 | 34.61 | C |
| ATOM | 5246 | CD2 | LEU | B | 515 | −5.527 | −5.925 | 75.059 | 1.00 | 34.62 | C |
| ATOM | 5247 | N | GLY | B | 516 | −2.150 | −5.952 | 79.397 | 1.00 | 34.10 | N |
| ATOM | 5248 | CA | GLY | B | 516 | −0.780 | −6.125 | 79.859 | 1.00 | 33.42 | C |
| ATOM | 5249 | C | GLY | B | 516 | −0.480 | −5.220 | 81.030 | 1.00 | 32.95 | C |
| ATOM | 5250 | O | GLY | B | 516 | −1.309 | −5.059 | 81.924 | 1.00 | 33.30 | O |
| ATOM | 5251 | N | ASN | B | 517 | 0.709 | −4.626 | 81.020 | 1.00 | 32.38 | N |
| ATOM | 5252 | CA | ASN | B | 517 | 1.170 | −3.769 | 82.110 | 1.00 | 31.66 | C |
| ATOM | 5253 | C | ASN | B | 517 | 2.218 | −2.781 | 81.612 | 1.00 | 31.45 | C |
| ATOM | 5254 | O | ASN | B | 517 | 3.134 | −3.151 | 80.870 | 1.00 | 31.50 | O |
| ATOM | 5255 | CB | ASN | B | 517 | 1.730 | −4.620 | 83.254 | 1.00 | 31.45 | C |
| ATOM | 5256 | CG | ASN | B | 517 | 2.042 | −3.811 | 84.503 | 1.00 | 30.97 | C |
| ATOM | 5257 | OD1 | ASN | B | 517 | 2.955 | −2.989 | 84.510 | 1.00 | 29.64 | O |
| ATOM | 5258 | ND2 | ASN | B | 517 | 1.296 | −4.064 | 85.576 | 1.00 | 29.73 | N |
| ATOM | 5259 | N | ILE | B | 518 | 2.084 | −1.530 | 82.042 | 1.00 | 31.00 | N |
| ATOM | 5260 | CA | ILE | B | 518 | 2.932 | −0.437 | 81.563 | 1.00 | 30.58 | C |
| ATOM | 5261 | C | ILE | B | 518 | 4.389 | −0.534 | 82.032 | 1.00 | 30.81 | C |
| ATOM | 5262 | O | ILE | B | 518 | 5.247 | 0.234 | 81.576 | 1.00 | 31.17 | O |
| ATOM | 5263 | CB | ILE | B | 518 | 2.336 | 0.951 | 81.923 | 1.00 | 30.31 | C |
| ATOM | 5264 | CG1 | ILE | B | 518 | 2.345 | 1.171 | 83.439 | 1.00 | 30.10 | C |
| ATOM | 5265 | CG2 | ILE | B | 518 | 0.936 | 1.092 | 81.339 | 1.00 | 29.38 | C |
| ATOM | 5266 | CD1 | ILE | B | 518 | 1.942 | 2.569 | 83.884 | 1.00 | 30.42 | C |
| ATOM | 5267 | N | TYR | B | 519 | 4.659 | −1.475 | 82.933 | 1.00 | 30.69 | N |
| ATOM | 5268 | CA | TYR | B | 519 | 5.994 | −1.672 | 83.488 | 1.00 | 30.74 | C |
| ATOM | 5269 | C | TYR | B | 519 | 6.738 | −2.835 | 82.815 | 1.00 | 30.92 | C |
| ATOM | 5270 | O | TYR | B | 519 | 7.910 | −3.082 | 83.109 | 1.00 | 30.80 | O |
| ATOM | 5271 | CB | TYR | B | 519 | 5.930 | −1.882 | 85.013 | 1.00 | 30.69 | C |
| ATOM | 5272 | CG | TYR | B | 519 | 5.191 | −0.803 | 85.797 | 1.00 | 30.58 | C |
| ATOM | 5273 | CD1 | TYR | B | 519 | 5.364 | 0.552 | 85.505 | 1.00 | 30.66 | C |
| ATOM | 5274 | CD2 | TYR | B | 519 | 4.342 | −1.141 | 86.853 | 1.00 | 30.48 | C |
| ATOM | 5275 | CE1 | TYR | B | 519 | 4.699 | 1.542 | 86.226 | 1.00 | 30.10 | C |
| ATOM | 5276 | CE2 | TYR | B | 519 | 3.668 | −0.157 | 87.581 | 1.00 | 30.91 | C |
| ATOM | 5277 | CZ | TYR | B | 519 | 3.855 | 1.184 | 87.259 | 1.00 | 30.90 | C |
| ATOM | 5278 | OH | TYR | B | 519 | 3.201 | 2.169 | 87.969 | 1.00 | 30.70 | O |
| ATOM | 5279 | N | PHE | B | 520 | 6.057 | −3.551 | 81.920 | 1.00 | 31.18 | N |
| ATOM | 5280 | CA | PHE | B | 520 | 6.697 | −4.623 | 81.155 | 1.00 | 31.43 | C |
| ATOM | 5281 | C | PHE | B | 520 | 7.464 | −4.030 | 79.981 | 1.00 | 31.82 | C |
| ATOM | 5282 | O | PHE | B | 520 | 7.004 | −3.071 | 79.358 | 1.00 | 31.76 | O |
| ATOM | 5283 | CB | PHE | B | 520 | 5.673 | −5.643 | 80.641 | 1.00 | 31.31 | C |
| ATOM | 5284 | CG | PHE | B | 520 | 4.875 | −6.332 | 81.727 | 1.00 | 31.23 | C |
| ATOM | 5285 | CD1 | PHE | B | 520 | 5.234 | −6.227 | 83.073 | 1.00 | 31.44 | C |
| ATOM | 5286 | CD2 | PHE | B | 520 | 3.773 | −7.110 | 81.393 | 1.00 | 30.66 | C |
| ATOM | 5287 | CE1 | PHE | B | 520 | 4.493 | −6.869 | 84.063 | 1.00 | 31.16 | C |
| ATOM | 5288 | CE2 | PHE | B | 520 | 3.031 | −7.760 | 82.373 | 1.00 | 30.76 | C |
| ATOM | 5289 | CZ | PHE | B | 520 | 3.390 | −7.640 | 83.711 | 1.00 | 31.21 | C |
| ATOM | 5290 | N | THR | B | 521 | 8.631 | −4.599 | 79.682 | 1.00 | 32.41 | N |
| ATOM | 5291 | CA | THR | B | 521 | 9.486 | −4.084 | 78.600 | 1.00 | 32.95 | C |
| ATOM | 5292 | C | THR | B | 521 | 9.806 | −5.122 | 77.519 | 1.00 | 33.22 | C |
| ATOM | 5293 | O | THR | B | 521 | 10.343 | −4.778 | 76.465 | 1.00 | 33.38 | O |
| ATOM | 5294 | CB | THR | B | 521 | 10.816 | −3.473 | 79.137 | 1.00 | 32.94 | C |
| ATOM | 5295 | OG1 | THR | B | 521 | 11.597 | −4.488 | 79.783 | 1.00 | 33.39 | O |
| ATOM | 5296 | CG2 | THR | B | 521 | 10.548 | −2.334 | 80.116 | 1.00 | 32.28 | C |
| ATOM | 5297 | N | THR | B | 522 | 9.452 | −6.378 | 77.778 | 1.00 | 33.59 | N |
| ATOM | 5298 | CA | THR | B | 522 | 9.831 | −7.511 | 76.928 | 1.00 | 33.90 | C |
| ATOM | 5299 | C | THR | B | 522 | 8.606 | −8.294 | 76.478 | 1.00 | 34.03 | C |
| ATOM | 5300 | O | THR | B | 522 | 7.668 | −8.489 | 77.259 | 1.00 | 34.18 | O |
| ATOM | 5301 | CB | THR | B | 522 | 10.793 | −8.452 | 77.699 | 1.00 | 33.89 | C |
| ATOM | 5302 | OG1 | THR | B | 522 | 12.080 | −7.832 | 77.799 | 1.00 | 34.50 | O |
| ATOM | 5303 | CG2 | THR | B | 522 | 10.949 | −9.807 | 77.021 | 1.00 | 34.51 | C |
| ATOM | 5304 | N | ASN | B | 523 | 8.617 | −8.744 | 75.223 | 1.00 | 34.27 | N |
| ATOM | 5305 | CA | ASN | B | 523 | 7.541 | −9.584 | 74.685 | 1.00 | 34.42 | C |
| ATOM | 5306 | C | ASN | B | 523 | 6.167 | −9.084 | 75.136 | 1.00 | 34.38 | C |
| ATOM | 5307 | O | ASN | B | 523 | 5.356 | −9.850 | 75.665 | 1.00 | 34.43 | O |
| ATOM | 5308 | CB | ASN | B | 523 | 7.734 | −11.051 | 75.109 | 1.00 | 34.57 | C |
| ATOM | 5309 | CG | ASN | B | 523 | 9.085 | −11.617 | 74.694 | 1.00 | 34.98 | C |
| ATOM | 5310 | OD1 | ASN | B | 523 | 9.706 | −11.152 | 73.734 | 1.00 | 36.33 | O |
| ATOM | 5311 | ND2 | ASN | B | 523 | 9.545 | −12.632 | 75.419 | 1.00 | 35.18 | N |
| ATOM | 5312 | N | LYS | B | 524 | 5.926 | −7.790 | 74.924 | 1.00 | 34.16 | N |
| ATOM | 5313 | CA | LYS | B | 524 | 4.756 | −7.105 | 75.468 | 1.00 | 33.85 | C |
| ATOM | 5314 | C | LYS | B | 524 | 3.437 | −7.592 | 74.884 | 1.00 | 34.02 | C |
| ATOM | 5315 | O | LYS | B | 524 | 3.383 | −8.039 | 73.740 | 1.00 | 33.94 | O |
| ATOM | 5316 | CB | LYS | B | 524 | 4.888 | −5.592 | 75.290 | 1.00 | 33.53 | C |
| ATOM | 5317 | CG | LYS | B | 524 | 5.763 | −4.921 | 76.339 | 1.00 | 32.35 | C |
| ATOM | 5318 | CD | LYS | B | 524 | 5.818 | −3.414 | 76.139 | 1.00 | 30.50 | C |
| ATOM | 5319 | CE | LYS | B | 524 | 4.507 | −2.738 | 76.525 | 1.00 | 29.53 | C |
| ATOM | 5320 | NZ | LYS | B | 524 | 4.206 | −2.814 | 77.981 | 1.00 | 28.19 | N |
| ATOM | 5321 | N | THR | B | 525 | 2.385 | −7.511 | 75.695 | 1.00 | 34.18 | N |
| ATOM | 5322 | CA | THR | B | 525 | 1.036 | −7.871 | 75.270 | 1.00 | 34.34 | C |
| ATOM | 5323 | C | THR | B | 525 | 0.130 | −6.638 | 75.133 | 1.00 | 34.07 | C |
| ATOM | 5324 | O | THR | B | 525 | −0.929 | −6.708 | 74.518 | 1.00 | 34.00 | O |
| ATOM | 5325 | CB | THR | B | 525 | 0.391 | −8.884 | 76.236 | 1.00 | 34.44 | C |
| ATOM | 5326 | OG1 | THR | B | 525 | 1.351 | −9.875 | 76.617 | 1.00 | 34.78 | O |
| ATOM | 5327 | CG2 | THR | B | 525 | −0.767 | −9.572 | 75.558 | 1.00 | 34.99 | C |
| ATOM | 5328 | N | SER | B | 526 | 0.551 | −5.521 | 75.724 | 1.00 | 33.94 | N |
| ATOM | 5329 | CA | SER | B | 526 | −0.145 | −4.245 | 75.579 | 1.00 | 33.71 | C |
| ATOM | 5330 | C | SER | B | 526 | 0.706 | −3.301 | 74.719 | 1.00 | 33.18 | C |
| ATOM | 5331 | O | SER | B | 526 | 1.911 | −3.515 | 74.598 | 1.00 | 33.43 | O |
| ATOM | 5332 | CB | SER | B | 526 | −0.426 | −3.637 | 76.956 | 1.00 | 33.82 | C |
| ATOM | 5333 | OG | SER | B | 526 | 0.769 | −3.191 | 77.578 | 1.00 | 34.67 | O |
| ATOM | 5334 | N | PRO | B | 527 | 0.091 | −2.267 | 74.105 | 1.00 | 32.71 | N |
| ATOM | 5335 | CA | PRO | B | 527 | 0.869 | −1.364 | 73.246 | 1.00 | 32.23 | C |
| ATOM | 5336 | C | PRO | B | 527 | 1.687 | −0.287 | 73.969 | 1.00 | 31.62 | C |
| ATOM | 5337 | O | PRO | B | 527 | 2.502 | 0.380 | 73.332 | 1.00 | 31.78 | O |
| ATOM | 5338 | CB | PRO | B | 527 | −0.202 | −0.699 | 72.359 | 1.00 | 32.21 | C |
| ATOM | 5339 | CG | PRO | B | 527 | −1.524 | −1.294 | 72.773 | 1.00 | 32.48 | C |
| ATOM | 5340 | CD | PRO | B | 527 | −1.329 | −1.880 | 74.130 | 1.00 | 32.67 | C |
| ATOM | 5341 | N | PHE | B | 528 | 1.491 | −0.123 | 75.275 | 1.00 | 30.93 | N |
| ATOM | 5342 | CA | PHE | B | 528 | 2.091 | 1.004 | 76.001 | 1.00 | 30.29 | C |
| ATOM | 5343 | C | PHE | B | 528 | 3.104 | 0.619 | 77.074 | 1.00 | 29.88 | C |
| ATOM | 5344 | O | PHE | B | 528 | 2.906 | −0.342 | 77.817 | 1.00 | 29.74 | O |
| ATOM | 5345 | CB | PHE | B | 528 | 1.003 | 1.867 | 76.646 | 1.00 | 30.08 | C |
| ATOM | 5346 | CG | PHE | B | 528 | 0.037 | 2.458 | 75.669 | 1.00 | 30.04 | C |
| ATOM | 5347 | CD1 | PHE | B | 528 | 0.362 | 3.613 | 74.963 | 1.00 | 29.60 | C |
| ATOM | 5348 | CD2 | PHE | B | 528 | −1.208 | 1.868 | 75.461 | 1.00 | 29.91 | C |
| ATOM | 5349 | CE1 | PHE | B | 528 | −0.533 | 4.170 | 74.056 | 1.00 | 29.89 | C |
| ATOM | 5350 | CE2 | PHE | B | 528 | −2.111 | 2.417 | 74.557 | 1.00 | 29.62 | C |
| ATOM | 5351 | CZ | PHE | B | 528 | −1.773 | 3.574 | 73.852 | 1.00 | 30.24 | C |
| ATOM | 5352 | N | ARG | B | 529 | 4.180 | 1.394 | 77.156 | 1.00 | 29.42 | N |
| ATOM | 5353 | CA | ARG | B | 529 | 5.128 | 1.284 | 78.262 | 1.00 | 29.03 | C |
| ATOM | 5354 | C | ARG | B | 529 | 5.547 | 2.670 | 78.745 | 1.00 | 28.74 | C |
| ATOM | 5355 | O | ARG | B | 529 | 5.469 | 3.650 | 77.999 | 1.00 | 28.80 | O |
| ATOM | 5356 | CB | ARG | B | 529 | 6.355 | 0.456 | 77.863 | 1.00 | 28.90 | C |
| ATOM | 5357 | CG | ARG | B | 529 | 7.106 | 0.993 | 76.654 | 1.00 | 29.06 | C |
| ATOM | 5358 | CD | ARG | B | 529 | 8.522 | 0.435 | 76.568 | 1.00 | 28.30 | C |
| ATOM | 5359 | NE | ARG | B | 529 | 9.268 | 1.086 | 75.494 | 1.00 | 28.03 | N |
| ATOM | 5360 | CZ | ARG | B | 529 | 9.974 | 2.212 | 75.626 | 1.00 | 27.68 | C |
| ATOM | 5361 | NH1 | ARG | B | 529 | 10.053 | 2.835 | 76.799 | 1.00 | 25.14 | N |
| ATOM | 5362 | NH2 | ARG | B | 529 | 10.606 | 2.718 | 74.573 | 1.00 | 27.73 | N |
| ATOM | 5363 | N | ILE | B | 530 | 5.976 | 2.753 | 79.997 | 1.00 | 28.32 | N |
| ATOM | 5364 | CA | ILE | B | 530 | 6.555 | 3.983 | 80.503 | 1.00 | 28.05 | C |
| ATOM | 5365 | C | ILE | B | 530 | 8.009 | 4.068 | 80.078 | 1.00 | 27.85 | C |
| ATOM | 5366 | O | ILE | B | 530 | 8.602 | 3.078 | 79.649 | 1.00 | 27.94 | O |
| ATOM | 5367 | CB | ILE | B | 530 | 6.484 | 4.084 | 82.047 | 1.00 | 28.18 | C |
| ATOM | 5368 | CG1 | ILE | B | 530 | 7.318 | 2.976 | 82.705 | 1.00 | 28.30 | C |
| ATOM | 5369 | CG2 | ILE | B | 530 | 5.028 | 4.101 | 82.526 | 1.00 | 28.04 | C |
| ATOM | 5370 | CD1 | ILE | B | 530 | 7.708 | 3.275 | 84.131 | 1.00 | 28.07 | C |
| ATOM | 5371 | N | ALA | B | 531 | 8.572 | 5.262 | 80.214 | 1.00 | 27.61 | N |
| ATOM | 5372 | CA | ALA | B | 531 | 9.984 | 5.503 | 79.986 | 1.00 | 27.33 | C |
| ATOM | 5373 | C | ALA | B | 531 | 10.866 | 4.522 | 80.761 | 1.00 | 27.22 | C |
| ATOM | 5374 | O | ALA | B | 531 | 10.607 | 4.241 | 81.936 | 1.00 | 27.10 | O |
| ATOM | 5375 | CB | ALA | B | 531 | 10.325 | 6.940 | 80.373 | 1.00 | 27.17 | C |
| ATOM | 5376 | N | LYS | B | 532 | 11.898 | 4.002 | 80.095 | 1.00 | 27.23 | N |
| ATOM | 5377 | CA | LYS | B | 532 | 12.960 | 3.256 | 80.771 | 1.00 | 27.52 | C |
| ATOM | 5378 | C | LYS | B | 532 | 13.844 | 4.233 | 81.539 | 1.00 | 27.37 | C |
| ATOM | 5379 | O | LYS | B | 532 | 14.741 | 4.858 | 80.967 | 1.00 | 27.57 | O |
| ATOM | 5380 | CB | LYS | B | 532 | 13.799 | 2.450 | 79.776 | 1.00 | 27.74 | C |
| ATOM | 5381 | CG | LYS | B | 532 | 13.186 | 1.122 | 79.369 | 1.00 | 29.16 | C |
| ATOM | 5382 | CD | LYS | B | 532 | 13.978 | 0.471 | 78.245 | 1.00 | 31.09 | C |
| ATOM | 5383 | CE | LYS | B | 532 | 13.184 | −0.656 | 77.592 | 1.00 | 31.74 | C |
| ATOM | 5384 | NZ | LYS | B | 532 | 13.701 | −0.934 | 76.222 | 1.00 | 32.70 | N |
| ATOM | 5385 | N | ASP | B | 533 | 13.567 | 4.364 | 82.834 | 1.00 | 27.13 | N |
| ATOM | 5386 | CA | ASP | B | 533 | 14.265 | 5.297 | 83.706 | 1.00 | 26.92 | C |
| ATOM | 5387 | C | ASP | B | 533 | 14.228 | 4.762 | 85.139 | 1.00 | 26.88 | C |
| ATOM | 5388 | O | ASP | B | 533 | 13.670 | 3.695 | 85.394 | 1.00 | 26.77 | O |
| ATOM | 5389 | CB | ASP | B | 533 | 13.592 | 6.673 | 83.630 | 1.00 | 27.05 | C |
| ATOM | 5390 | CG | ASP | B | 533 | 14.475 | 7.800 | 84.160 | 1.00 | 27.62 | C |
| ATOM | 5391 | OD1 | ASP | B | 533 | 15.694 | 7.592 | 84.344 | 1.00 | 28.37 | O |
| ATOM | 5392 | OD2 | ASP | B | 533 | 13.943 | 8.907 | 84.388 | 1.00 | 28.31 | O |
| ATOM | 5393 | N | SER | B | 534 | 14.837 | 5.496 | 86.065 | 1.00 | 26.89 | N |
| ATOM | 5394 | CA | SER | B | 534 | 14.733 | 5.184 | 87.480 | 1.00 | 26.99 | C |
| ATOM | 5395 | C | SER | B | 534 | 13.672 | 6.073 | 88.115 | 1.00 | 27.04 | C |
| ATOM | 5396 | O | SER | B | 534 | 13.558 | 7.262 | 87.803 | 1.00 | 26.95 | O |
| ATOM | 5397 | CB | SER | B | 534 | 16.079 | 5.355 | 88.192 | 1.00 | 27.02 | C |
| ATOM | 5398 | OG | SER | B | 534 | 16.207 | 6.634 | 88.797 | 1.00 | 27.52 | O |
| ATOM | 5399 | N | TYR | B | 535 | 12.895 | 5.485 | 89.012 | 1.00 | 27.30 | N |
| ATOM | 5400 | CA | TYR | B | 535 | 11.815 | 6.202 | 89.656 | 1.00 | 27.55 | C |
| ATOM | 5401 | C | TYR | B | 535 | 11.922 | 6.074 | 91.166 | 1.00 | 28.22 | C |
| ATOM | 5402 | O | TYR | B | 535 | 12.625 | 5.198 | 91.676 | 1.00 | 28.48 | O |
| ATOM | 5403 | CB | TYR | B | 535 | 10.465 | 5.680 | 89.171 | 1.00 | 26.88 | C |
| ATOM | 5404 | CG | TYR | B | 535 | 10.210 | 5.862 | 87.697 | 1.00 | 26.03 | C |
| ATOM | 5405 | CD1 | TYR | B | 535 | 10.622 | 4.900 | 86.767 | 1.00 | 25.26 | C |
| ATOM | 5406 | CD2 | TYR | B | 535 | 9.538 | 6.984 | 87.229 | 1.00 | 25.95 | C |
| ATOM | 5407 | CE1 | TYR | B | 535 | 10.380 | 5.066 | 85.412 | 1.00 | 25.04 | C |
| ATOM | 5408 | CE2 | TYR | B | 535 | 9.283 | 7.159 | 85.868 | 1.00 | 25.76 | C |
| ATOM | 5409 | CZ | TYR | B | 535 | 9.704 | 6.199 | 84.968 | 1.00 | 25.57 | C |
| ATOM | 5410 | OH | TYR | B | 535 | 9.446 | 6.378 | 83.628 | 1.00 | 25.40 | O |
| ATOM | 5411 | N | LEU | B | 536 | 11.216 | 6.954 | 91.870 | 1.00 | 28.94 | N |
| ATOM | 5412 | CA | LEU | B | 536 | 11.231 | 6.992 | 93.321 | 1.00 | 29.85 | C |
| ATOM | 5413 | C | LEU | B | 536 | 9.800 | 6.982 | 93.850 | 1.00 | 30.61 | C |
| ATOM | 5414 | O | LEU | B | 536 | 9.044 | 7.935 | 93.647 | 1.00 | 30.76 | O |
| ATOM | 5415 | CB | LEU | B | 536 | 11.980 | 8.238 | 93.801 | 1.00 | 29.68 | C |
| ATOM | 5416 | CG | LEU | B | 536 | 12.635 | 8.193 | 95.179 | 1.00 | 29.83 | C |
| ATOM | 5417 | CD1 | LEU | B | 536 | 13.870 | 7.304 | 95.168 | 1.00 | 30.44 | C |
| ATOM | 5418 | CD2 | LEU | B | 536 | 12.997 | 9.594 | 95.625 | 1.00 | 29.58 | C |
| ATOM | 5419 | N | TRP | B | 537 | 9.441 | 5.896 | 94.526 | 1.00 | 31.50 | N |
| ATOM | 5420 | CA | TRP | B | 537 | 8.086 | 5.687 | 95.033 | 1.00 | 32.36 | C |
| ATOM | 5421 | C | TRP | B | 537 | 7.993 | 5.828 | 96.554 | 1.00 | 33.62 | C |
| ATOM | 5422 | O | TRP | B | 537 | 8.997 | 5.701 | 97.265 | 1.00 | 33.77 | O |
| ATOM | 5423 | CB | TRP | B | 537 | 7.588 | 4.304 | 94.604 | 1.00 | 31.66 | C |
| ATOM | 5424 | CG | TRP | B | 537 | 7.194 | 4.221 | 93.159 | 1.00 | 30.85 | C |
| ATOM | 5425 | CD1 | TRP | B | 537 | 7.418 | 5.159 | 92.190 | 1.00 | 30.22 | C |
| ATOM | 5426 | CD2 | TRP | B | 537 | 6.535 | 3.131 | 92.514 | 1.00 | 29.72 | C |
| ATOM | 5427 | NE1 | TRP | B | 537 | 6.924 | 4.728 | 90.990 | 1.00 | 29.84 | N |
| ATOM | 5428 | CE2 | TRP | B | 537 | 6.372 | 3.485 | 91.158 | 1.00 | 30.25 | C |
| ATOM | 5429 | CE3 | TRP | B | 537 | 6.055 | 1.890 | 92.950 | 1.00 | 29.73 | C |
| ATOM | 5430 | CZ2 | TRP | B | 537 | 5.750 | 2.640 | 90.231 | 1.00 | 30.26 | C |
| ATOM | 5431 | CZ3 | TRP | B | 537 | 5.437 | 1.053 | 92.033 | 1.00 | 29.76 | C |
| ATOM | 5432 | CH2 | TRP | B | 537 | 5.293 | 1.432 | 90.688 | 1.00 | 30.54 | C |
| ATOM | 5433 | N | MET | B | 538 | 6.781 | 6.092 | 97.042 | 1.00 | 35.08 | N |
| ATOM | 5434 | CA | MET | B | 538 | 6.518 | 6.198 | 98.476 | 1.00 | 36.64 | C |
| ATOM | 5435 | C | MET | B | 538 | 5.208 | 5.531 | 98.888 | 1.00 | 37.14 | C |
| ATOM | 5436 | O | MET | B | 538 | 4.179 | 5.689 | 98.228 | 1.00 | 37.36 | O |
| ATOM | 5437 | CB | MET | B | 538 | 6.495 | 7.662 | 98.919 | 1.00 | 36.53 | C |
| ATOM | 5438 | CG | MET | B | 538 | 6.274 | 7.844 | 100.419 | 1.00 | 37.08 | C |
| ATOM | 5439 | SD | MET | B | 538 | 5.515 | 9.420 | 100.833 | 1.00 | 38.25 | S |
| ATOM | 5440 | CE | MET | B | 538 | 3.800 | 9.065 | 100.473 | 1.00 | 37.85 | C |
| ATOM | 5441 | N | SER | B | 539 | 5.265 | 4.789 | 99.990 | 1.00 | 37.92 | N |
| ATOM | 5442 | CA | SER | B | 539 | 4.074 | 4.265 | 100.641 | 1.00 | 38.71 | C |
| ATOM | 5443 | C | SER | B | 539 | 4.060 | 4.702 | 102.101 | 1.00 | 39.46 | C |
| ATOM | 5444 | O | SER | B | 539 | 5.120 | 4.926 | 102.701 | 1.00 | 39.28 | O |
| ATOM | 5445 | CB | SER | B | 539 | 4.044 | 2.746 | 100.547 | 1.00 | 38.62 | C |
| ATOM | 5446 | OG | SER | B | 539 | 4.047 | 2.329 | 99.196 | 1.00 | 38.35 | O |
| ATOM | 5447 | N | TYR | B | 540 | 2.863 | 4.833 | 102.669 | 1.00 | 40.46 | N |
| ATOM | 5448 | CA | TYR | B | 540 | 2.727 | 5.210 | 104.079 | 1.00 | 41.43 | C |
| ATOM | 5449 | C | TYR | B | 540 | 1.664 | 4.404 | 104.820 | 1.00 | 42.03 | C |
| ATOM | 5450 | O | TYR | B | 540 | 0.700 | 3.916 | 104.213 | 1.00 | 42.09 | O |
| ATOM | 5451 | CB | TYR | B | 540 | 2.463 | 6.713 | 104.228 | 1.00 | 41.37 | C |
| ATOM | 5452 | CG | TYR | B | 540 | 1.190 | 7.206 | 103.573 | 1.00 | 41.80 | C |
| ATOM | 5453 | CD1 | TYR | B | 540 | −0.030 | 7.194 | 104.261 | 1.00 | 42.26 | C |
| ATOM | 5454 | CD2 | TYR | B | 540 | 1.205 | 7.704 | 102.271 | 1.00 | 41.46 | C |
| ATOM | 5455 | CE1 | TYR | B | 540 | −1.201 | 7.657 | 103.658 | 1.00 | 41.96 | C |
| ATOM | 5456 | CE2 | TYR | B | 540 | 0.048 | 8.167 | 101.662 | 1.00 | 41.23 | C |
| ATOM | 5457 | CZ | TYR | B | 540 | −1.148 | 8.142 | 102.357 | 1.00 | 41.85 | C |
| ATOM | 5458 | OH | TYR | B | 540 | −2.290 | 8.601 | 101.747 | 1.00 | 42.44 | O |
| ATOM | 5459 | N | SER | B | 541 | 1.865 | 4.262 | 106.131 | 1.00 | 42.62 | N |
| ATOM | 5460 | CA | SER | B | 541 | 0.894 | 3.620 | 107.011 | 1.00 | 43.30 | C |
| ATOM | 5461 | C | SER | B | 541 | 0.613 | 4.511 | 108.212 | 1.00 | 43.76 | C |
| ATOM | 5462 | O | SER | B | 541 | 1.536 | 5.081 | 108.801 | 1.00 | 43.77 | O |
| ATOM | 5463 | CB | SER | B | 541 | 1.406 | 2.263 | 107.486 | 1.00 | 43.31 | C |
| ATOM | 5464 | OG | SER | B | 541 | 2.179 | 2.396 | 108.665 | 1.00 | 43.97 | O |
| ATOM | 5465 | N | ASP | B | 542 | −0.664 | 4.624 | 108.565 | 1.00 | 44.48 | N |
| ATOM | 5466 | CA | ASP | B | 542 | −1.095 | 5.412 | 109.719 | 1.00 | 44.93 | C |
| ATOM | 5467 | C | ASP | B | 542 | −1.414 | 4.525 | 110.922 | 1.00 | 45.32 | C |
| ATOM | 5468 | O | ASP | B | 542 | −1.502 | 5.008 | 112.050 | 1.00 | 45.64 | O |
| ATOM | 5469 | CB | ASP | B | 542 | −2.310 | 6.266 | 109.354 | 1.00 | 45.00 | C |
| ATOM | 5470 | CG | ASP | B | 542 | −1.972 | 7.380 | 108.372 | 1.00 | 45.18 | C |
| ATOM | 5471 | OD1 | ASP | B | 542 | −1.051 | 8.177 | 108.658 | 1.00 | 45.15 | O |
| ATOM | 5472 | OD2 | ASP | B | 542 | −2.639 | 7.468 | 107.318 | 1.00 | 45.10 | O |
| ATOM | 5473 | N | ASP | B | 543 | −1.583 | 3.228 | 110.674 | 1.00 | 45.65 | N |
| ATOM | 5474 | CA | ASP | B | 543 | −1.896 | 2.265 | 111.726 | 1.00 | 45.81 | C |
| ATOM | 5475 | C | ASP | B | 543 | −0.652 | 1.551 | 112.248 | 1.00 | 45.92 | C |
| ATOM | 5476 | O | ASP | B | 543 | −0.669 | 0.338 | 112.469 | 1.00 | 46.04 | O |
| ATOM | 5477 | CB | ASP | B | 543 | −2.933 | 1.245 | 111.236 | 1.00 | 45.94 | C |
| ATOM | 5478 | CG | ASP | B | 543 | −2.579 | 0.634 | 109.878 | 1.00 | 46.38 | C |
| ATOM | 5479 | OD1 | ASP | B | 543 | −1.377 | 0.501 | 109.544 | 1.00 | 46.82 | O |
| ATOM | 5480 | OD2 | ASP | B | 543 | −3.522 | 0.279 | 109.139 | 1.00 | 46.36 | O |
| ATOM | 5481 | N | ASP | B | 544 | 0.428 | 2.310 | 112.427 | 1.00 | 46.12 | N |
| ATOM | 5482 | CA | ASP | B | 544 | 1.678 | 1.808 | 113.016 | 1.00 | 46.28 | C |
| ATOM | 5483 | C | ASP | B | 544 | 2.313 | 0.639 | 112.231 | 1.00 | 46.26 | C |
| ATOM | 5484 | O | ASP | B | 544 | 3.132 | −0.117 | 112.768 | 1.00 | 46.14 | O |
| ATOM | 5485 | CB | ASP | B | 544 | 1.464 | 1.461 | 114.504 | 1.00 | 46.44 | C |
| ATOM | 5486 | CG | ASP | B | 544 | 2.764 | 1.202 | 115.249 | 1.00 | 46.85 | C |
| ATOM | 5487 | OD1 | ASP | B | 544 | 3.626 | 2.109 | 115.331 | 1.00 | 46.65 | O |
| ATOM | 5488 | OD2 | ASP | B | 544 | 2.911 | 0.076 | 115.763 | 1.00 | 47.47 | O |
| ATOM | 5489 | N | GLY | B | 545 | 1.930 | 0.502 | 110.959 | 1.00 | 46.26 | N |
| ATOM | 5490 | CA | GLY | B | 545 | 2.591 | −0.427 | 110.037 | 1.00 | 45.98 | C |
| ATOM | 5491 | C | GLY | B | 545 | 1.849 | −1.696 | 109.662 | 1.00 | 45.95 | C |
| ATOM | 5492 | O | GLY | B | 545 | 2.439 | −2.599 | 109.074 | 1.00 | 45.95 | O |
| ATOM | 5493 | N | LYS | B | 546 | 0.558 | −1.770 | 109.983 | 1.00 | 45.80 | N |
| ATOM | 5494 | CA | LYS | B | 546 | −0.235 | −2.968 | 109.686 | 1.00 | 45.64 | C |
| ATOM | 5495 | C | LYS | B | 546 | −0.792 | −2.989 | 108.255 | 1.00 | 45.23 | C |
| ATOM | 5496 | O | LYS | B | 546 | −0.761 | −4.028 | 107.593 | 1.00 | 45.25 | O |
| ATOM | 5497 | CB | LYS | B | 546 | −1.341 | −3.160 | 110.728 | 1.00 | 45.92 | C |
| ATOM | 5498 | CG | LYS | B | 546 | −0.808 | −3.514 | 112.117 | 1.00 | 46.58 | C |
| ATOM | 5499 | CD | LYS | B | 546 | −1.780 | −3.109 | 113.212 | 1.00 | 47.97 | C |
| ATOM | 5500 | CE | LYS | B | 546 | −1.141 | −3.240 | 114.590 | 1.00 | 49.01 | C |
| ATOM | 5501 | NZ | LYS | B | 546 | −1.980 | −2.623 | 115.657 | 1.00 | 49.63 | N |
| ATOM | 5502 | N | THR | B | 547 | −1.312 | −1.856 | 107.789 | 1.00 | 44.66 | N |
| ATOM | 5503 | CA | THR | B | 547 | −1.633 | −1.689 | 106.366 | 1.00 | 44.11 | C |
| ATOM | 5504 | C | THR | B | 547 | −0.885 | −0.491 | 105.792 | 1.00 | 43.51 | C |
| ATOM | 5505 | O | THR | B | 547 | −0.496 | 0.418 | 106.530 | 1.00 | 43.51 | O |
| ATOM | 5506 | CB | THR | B | 547 | −3.149 | −1.522 | 106.086 | 1.00 | 44.17 | C |
| ATOM | 5507 | OG1 | THR | B | 547 | −3.645 | −0.356 | 106.758 | 1.00 | 44.21 | O |
| ATOM | 5508 | CG2 | THR | B | 547 | −3.930 | −2.757 | 106.524 | 1.00 | 44.45 | C |
| ATOM | 5509 | N | TRP | B | 548 | −0.695 | −0.502 | 104.474 | 1.00 | 42.60 | N |
| ATOM | 5510 | CA | TRP | B | 548 | 0.045 | 0.538 | 103.766 | 1.00 | 41.61 | C |
| ATOM | 5511 | C | TRP | B | 548 | −0.754 | 1.025 | 102.566 | 1.00 | 40.94 | C |
| ATOM | 5512 | O | TRP | B | 548 | −1.451 | 0.237 | 101.923 | 1.00 | 40.89 | O |
| ATOM | 5513 | CB | TRP | B | 548 | 1.391 | −0.011 | 103.286 | 1.00 | 41.66 | C |
| ATOM | 5514 | CG | TRP | B | 548 | 2.340 | −0.333 | 104.398 | 1.00 | 41.64 | C |
| ATOM | 5515 | CD1 | TRP | B | 548 | 2.408 | −1.496 | 105.101 | 1.00 | 41.83 | C |
| ATOM | 5516 | CD2 | TRP | B | 548 | 3.359 | 0.522 | 104.933 | 1.00 | 41.66 | C |
| ATOM | 5517 | NE1 | TRP | B | 548 | 3.403 | −1.422 | 106.045 | 1.00 | 42.15 | N |
| ATOM | 5518 | CE2 | TRP | B | 548 | 4.005 | −0.195 | 105.962 | 1.00 | 41.76 | C |
| ATOM | 5519 | CE3 | TRP | B | 548 | 3.790 | 1.822 | 104.640 | 1.00 | 42.02 | C |
| ATOM | 5520 | CZ2 | TRP | B | 548 | 5.057 | 0.345 | 106.708 | 1.00 | 41.69 | C |
| ATOM | 5521 | CZ3 | TRP | B | 548 | 4.838 | 2.360 | 105.379 | 1.00 | 41.99 | C |
| ATOM | 5522 | CH2 | TRP | B | 548 | 5.458 | 1.620 | 106.403 | 1.00 | 42.00 | C |
| ATOM | 5523 | N | SER | B | 549 | −0.642 | 2.316 | 102.256 | 1.00 | 39.99 | N |
| ATOM | 5524 | CA | SER | B | 549 | −1.311 | 2.886 | 101.084 | 1.00 | 39.18 | C |
| ATOM | 5525 | C | SER | B | 549 | −0.731 | 2.333 | 99.778 | 1.00 | 38.67 | C |
| ATOM | 5526 | O | SER | B | 549 | 0.294 | 1.642 | 99.779 | 1.00 | 38.54 | O |
| ATOM | 5527 | CB | SER | B | 549 | −1.206 | 4.414 | 101.090 | 1.00 | 39.21 | C |
| ATOM | 5528 | OG | SER | B | 549 | 0.113 | 4.834 | 100.786 | 1.00 | 38.87 | O |
| ATOM | 5529 | N | ALA | B | 550 | −1.401 | 2.634 | 98.670 | 1.00 | 37.88 | N |
| ATOM | 5530 | CA | ALA | B | 550 | −0.861 | 2.353 | 97.346 | 1.00 | 37.19 | C |
| ATOM | 5531 | C | ALA | B | 550 | 0.369 | 3.229 | 97.128 | 1.00 | 36.69 | C |
| ATOM | 5532 | O | ALA | B | 550 | 0.452 | 4.323 | 97.694 | 1.00 | 36.53 | O |
| ATOM | 5533 | CB | ALA | B | 550 | −1.902 | 2.621 | 96.285 | 1.00 | 37.15 | C |
| ATOM | 5534 | N | PRO | B | 551 | 1.336 | 2.749 | 96.322 | 1.00 | 36.11 | N |
| ATOM | 5535 | CA | PRO | B | 551 | 2.538 | 3.532 | 96.068 | 1.00 | 35.70 | C |
| ATOM | 5536 | C | PRO | B | 551 | 2.190 | 4.884 | 95.470 | 1.00 | 35.24 | C |
| ATOM | 5537 | O | PRO | B | 551 | 1.366 | 4.964 | 94.560 | 1.00 | 35.35 | O |
| ATOM | 5538 | CB | PRO | B | 551 | 3.291 | 2.698 | 95.031 | 1.00 | 35.64 | C |
| ATOM | 5539 | CG | PRO | B | 551 | 2.786 | 1.330 | 95.197 | 1.00 | 36.01 | C |
| ATOM | 5540 | CD | PRO | B | 551 | 1.355 | 1.469 | 95.594 | 1.00 | 36.10 | C |
| ATOM | 5541 | N | GLN | B | 552 | 2.792 | 5.935 | 96.010 | 1.00 | 34.81 | N |
| ATOM | 5542 | CA | GLN | B | 552 | 2.703 | 7.262 | 95.424 | 1.00 | 34.41 | C |
| ATOM | 5543 | C | GLN | B | 552 | 4.005 | 7.478 | 94.659 | 1.00 | 34.06 | C |
| ATOM | 5544 | O | GLN | B | 552 | 5.078 | 7.071 | 95.121 | 1.00 | 33.95 | O |
| ATOM | 5545 | CB | GLN | B | 552 | 2.530 | 8.340 | 96.505 | 1.00 | 34.46 | C |
| ATOM | 5546 | CG | GLN | B | 552 | 1.624 | 7.956 | 97.689 | 1.00 | 34.73 | C |
| ATOM | 5547 | CD | GLN | B | 552 | 0.135 | 7.981 | 97.358 | 1.00 | 34.94 | C |
| ATOM | 5548 | OE1 | GLN | B | 552 | −0.472 | 9.046 | 97.258 | 1.00 | 34.95 | O |
| ATOM | 5549 | NE2 | GLN | B | 552 | −0.461 | 6.800 | 97.213 | 1.00 | 34.68 | N |
| ATOM | 5550 | N | ASP | B | 553 | 3.914 | 8.089 | 93.482 | 1.00 | 33.43 | N |
| ATOM | 5551 | CA | ASP | B | 553 | 5.103 | 8.367 | 92.689 | 1.00 | 32.67 | C |
| ATOM | 5552 | C | ASP | B | 553 | 5.588 | 9.777 | 92.997 | 1.00 | 32.23 | C |
| ATOM | 5553 | O | ASP | B | 553 | 4.921 | 10.765 | 92.654 | 1.00 | 32.31 | O |
| ATOM | 5554 | CB | ASP | B | 553 | 4.813 | 8.192 | 91.198 | 1.00 | 32.69 | C |
| ATOM | 5555 | CG | ASP | B | 553 | 6.019 | 8.506 | 90.315 | 1.00 | 32.97 | C |
| ATOM | 5556 | OD1 | ASP | B | 553 | 7.174 | 8.515 | 90.805 | 1.00 | 33.10 | O |
| ATOM | 5557 | OD2 | ASP | B | 553 | 5.798 | 8.740 | 89.113 | 1.00 | 32.29 | O |
| ATOM | 5558 | N | ILE | B | 554 | 6.742 | 9.867 | 93.654 | 1.00 | 31.24 | N |
| ATOM | 5559 | CA | ILE | B | 554 | 7.288 | 11.167 | 94.039 | 1.00 | 30.38 | C |
| ATOM | 5560 | C | ILE | B | 554 | 8.381 | 11.675 | 93.087 | 1.00 | 29.89 | C |
| ATOM | 5561 | O | ILE | B | 554 | 8.835 | 12.815 | 93.206 | 1.00 | 29.62 | O |
| ATOM | 5562 | CB | ILE | B | 554 | 7.731 | 11.213 | 95.539 | 1.00 | 30.30 | C |
| ATOM | 5563 | CG1 | ILE | B | 554 | 8.881 | 10.241 | 95.827 | 1.00 | 30.21 | C |
| ATOM | 5564 | CG2 | ILE | B | 554 | 6.530 | 10.943 | 96.457 | 1.00 | 30.40 | C |
| ATOM | 5565 | CD1 | ILE | B | 554 | 9.570 | 10.458 | 97.176 | 1.00 | 29.97 | C |
| ATOM | 5566 | N | THR | B | 555 | 8.766 | 10.832 | 92.127 | 1.00 | 29.44 | N |
| ATOM | 5567 | CA | THR | B | 555 | 9.842 | 11.136 | 91.171 | 1.00 | 28.88 | C |
| ATOM | 5568 | C | THR | B | 555 | 9.834 | 12.583 | 90.639 | 1.00 | 28.68 | C |
| ATOM | 5569 | O | THR | B | 555 | 10.850 | 13.263 | 90.767 | 1.00 | 28.71 | O |
| ATOM | 5570 | CB | THR | B | 555 | 9.884 | 10.119 | 89.991 | 1.00 | 28.94 | C |
| ATOM | 5571 | OG1 | THR | B | 555 | 9.828 | 8.785 | 90.503 | 1.00 | 28.85 | O |
| ATOM | 5572 | CG2 | THR | B | 555 | 11.152 | 10.276 | 89.167 | 1.00 | 28.42 | C |
| ATOM | 5573 | N | PRO | B | 556 | 8.695 | 13.059 | 90.066 | 1.00 | 28.47 | N |
| ATOM | 5574 | CA | PRO | B | 556 | 8.679 | 14.373 | 89.401 | 1.00 | 28.29 | C |
| ATOM | 5575 | C | PRO | B | 556 | 8.890 | 15.578 | 90.311 | 1.00 | 28.20 | C |
| ATOM | 5576 | O | PRO | B | 556 | 9.163 | 16.676 | 89.825 | 1.00 | 28.22 | O |
| ATOM | 5577 | CB | PRO | B | 556 | 7.281 | 14.432 | 88.776 | 1.00 | 28.42 | C |
| ATOM | 5578 | CG | PRO | B | 556 | 6.456 | 13.526 | 89.614 | 1.00 | 28.24 | C |
| ATOM | 5579 | CD | PRO | B | 556 | 7.370 | 12.409 | 89.987 | 1.00 | 28.41 | C |
| ATOM | 5580 | N | MET | B | 557 | 8.761 | 15.384 | 91.616 | 1.00 | 28.23 | N |
| ATOM | 5581 | CA | MET | B | 557 | 8.965 | 16.482 | 92.556 | 1.00 | 28.15 | C |
| ATOM | 5582 | C | MET | B | 557 | 10.453 | 16.718 | 92.815 | 1.00 | 27.52 | C |
| ATOM | 5583 | O | MET | B | 557 | 10.858 | 17.830 | 93.161 | 1.00 | 27.07 | O |
| ATOM | 5584 | CB | MET | B | 557 | 8.231 | 16.208 | 93.869 | 1.00 | 28.24 | C |
| ATOM | 5585 | CG | MET | B | 557 | 6.729 | 16.481 | 93.832 | 1.00 | 28.62 | C |
| ATOM | 5586 | SD | MET | B | 557 | 5.909 | 16.064 | 95.396 | 1.00 | 29.66 | S |
| ATOM | 5587 | CE | MET | B | 557 | 5.738 | 14.294 | 95.238 | 1.00 | 28.01 | C |
| ATOM | 5588 | N | VAL | B | 558 | 11.258 | 15.673 | 92.614 | 1.00 | 27.07 | N |
| ATOM | 5589 | CA | VAL | B | 558 | 12.662 | 15.668 | 93.038 | 1.00 | 26.68 | C |
| ATOM | 5590 | C | VAL | B | 558 | 13.706 | 15.391 | 91.940 | 1.00 | 26.51 | C |
| ATOM | 5591 | O | VAL | B | 558 | 14.840 | 15.872 | 92.036 | 1.00 | 26.47 | O |
| ATOM | 5592 | CB | VAL | B | 558 | 12.890 | 14.673 | 94.217 | 1.00 | 26.64 | C |
| ATOM | 5593 | CG1 | VAL | B | 558 | 12.206 | 15.173 | 95.479 | 1.00 | 26.38 | C |
| ATOM | 5594 | CG2 | VAL | B | 558 | 12.405 | 13.265 | 93.855 | 1.00 | 25.98 | C |
| ATOM | 5595 | N | LYS | B | 559 | 13.333 | 14.609 | 90.925 | 1.00 | 26.17 | N |
| ATOM | 5596 | CA | LYS | B | 559 | 14.262 | 14.191 | 89.864 | 1.00 | 25.91 | C |
| ATOM | 5597 | C | LYS | B | 559 | 14.408 | 15.243 | 88.764 | 1.00 | 25.71 | C |
| ATOM | 5598 | O | LYS | B | 559 | 13.491 | 15.450 | 87.972 | 1.00 | 25.69 | O |
| ATOM | 5599 | CB | LYS | B | 559 | 13.813 | 12.857 | 89.250 | 1.00 | 26.06 | C |
| ATOM | 5600 | CG | LYS | B | 559 | 14.867 | 12.153 | 88.406 | 1.00 | 25.24 | C |
| ATOM | 5601 | CD | LYS | B | 559 | 14.416 | 10.760 | 88.044 | 1.00 | 25.30 | C |
| ATOM | 5602 | CE | LYS | B | 559 | 15.511 | 9.971 | 87.350 | 1.00 | 26.33 | C |
| ATOM | 5603 | NZ | LYS | B | 559 | 15.695 | 10.363 | 85.920 | 1.00 | 26.55 | N |
| ATOM | 5604 | N | ALA | B | 560 | 15.571 | 15.890 | 88.720 | 1.00 | 25.47 | N |
| ATOM | 5605 | CA | ALA | B | 560 | 15.885 | 16.895 | 87.695 | 1.00 | 25.16 | C |
| ATOM | 5606 | C | ALA | B | 560 | 16.142 | 16.232 | 86.338 | 1.00 | 24.97 | C |
| ATOM | 5607 | O | ALA | B | 560 | 16.381 | 15.024 | 86.266 | 1.00 | 24.80 | O |
| ATOM | 5608 | CB | ALA | B | 560 | 17.090 | 17.741 | 88.122 | 1.00 | 24.79 | C |
| ATOM | 5609 | N | ASP | B | 561 | 16.101 | 17.029 | 85.272 | 1.00 | 24.81 | N |
| ATOM | 5610 | CA | ASP | B | 561 | 16.168 | 16.505 | 83.905 | 1.00 | 24.95 | C |
| ATOM | 5611 | C | ASP | B | 561 | 17.455 | 15.750 | 83.572 | 1.00 | 24.74 | C |
| ATOM | 5612 | O | ASP | B | 561 | 17.416 | 14.750 | 82.850 | 1.00 | 24.57 | O |
| ATOM | 5613 | CB | ASP | B | 561 | 15.919 | 17.620 | 82.889 | 1.00 | 25.00 | C |
| ATOM | 5614 | CG | ASP | B | 561 | 14.494 | 18.114 | 82.919 | 1.00 | 26.57 | C |
| ATOM | 5615 | OD1 | ASP | B | 561 | 13.606 | 17.343 | 83.353 | 1.00 | 29.21 | O |
| ATOM | 5616 | OD2 | ASP | B | 561 | 14.252 | 19.272 | 82.520 | 1.00 | 28.90 | O |
| ATOM | 5617 | N | TRP | B | 562 | 18.575 | 16.230 | 84.113 | 1.00 | 24.45 | N |
| ATOM | 5618 | CA | TRP | B | 562 | 19.895 | 15.664 | 83.838 | 1.00 | 24.21 | C |
| ATOM | 5619 | C | TRP | B | 562 | 20.142 | 14.347 | 84.569 | 1.00 | 24.31 | C |
| ATOM | 5620 | O | TRP | B | 562 | 21.037 | 13.590 | 84.199 | 1.00 | 24.61 | O |
| ATOM | 5621 | CB | TRP | B | 562 | 20.998 | 16.673 | 84.187 | 1.00 | 24.00 | C |
| ATOM | 5622 | CG | TRP | B | 562 | 21.061 | 17.071 | 85.653 | 1.00 | 23.80 | C |
| ATOM | 5623 | CD1 | TRP | B | 562 | 20.578 | 18.223 | 86.207 | 1.00 | 23.71 | C |
| ATOM | 5624 | CD2 | TRP | B | 562 | 21.635 | 16.317 | 86.737 | 1.00 | 23.72 | C |
| ATOM | 5625 | NE1 | TRP | B | 562 | 20.821 | 18.241 | 87.563 | 1.00 | 24.04 | N |
| ATOM | 5626 | CE2 | TRP | B | 562 | 21.470 | 17.086 | 87.914 | 1.00 | 24.19 | C |
| ATOM | 5627 | CE3 | TRP | B | 562 | 22.281 | 15.074 | 86.827 | 1.00 | 23.67 | C |
| ATOM | 5628 | CZ2 | TRP | B | 562 | 21.922 | 16.650 | 89.165 | 1.00 | 23.74 | C |
| ATOM | 5629 | CZ3 | TRP | B | 562 | 22.727 | 14.640 | 88.073 | 1.00 | 23.73 | C |
| ATOM | 5630 | CH2 | TRP | B | 562 | 22.548 | 15.430 | 89.224 | 1.00 | 23.92 | C |
| ATOM | 5631 | N | MET | B | 563 | 19.361 | 14.082 | 85.613 | 1.00 | 24.22 | N |
| ATOM | 5632 | CA | MET | B | 563 | 19.567 | 12.896 | 86.440 | 1.00 | 24.09 | C |
| ATOM | 5633 | C | MET | B | 563 | 19.247 | 11.612 | 85.684 | 1.00 | 24.61 | C |
| ATOM | 5634 | O | MET | B | 563 | 18.193 | 11.495 | 85.045 | 1.00 | 24.33 | O |
| ATOM | 5635 | CB | MET | B | 563 | 18.750 | 12.973 | 87.734 | 1.00 | 23.82 | C |
| ATOM | 5636 | CG | MET | B | 563 | 19.249 | 13.997 | 88.739 | 1.00 | 23.06 | C |
| ATOM | 5637 | SD | MET | B | 563 | 18.109 | 14.161 | 90.125 | 1.00 | 23.74 | S |
| ATOM | 5638 | CE | MET | B | 563 | 18.794 | 15.557 | 91.017 | 1.00 | 23.20 | C |
| ATOM | 5639 | N | LYS | B | 564 | 20.181 | 10.662 | 85.754 | 1.00 | 25.02 | N |
| ATOM | 5640 | CA | LYS | B | 564 | 19.978 | 9.321 | 85.222 | 1.00 | 25.42 | C |
| ATOM | 5641 | C | LYS | B | 564 | 19.348 | 8.465 | 86.323 | 1.00 | 25.72 | C |
| ATOM | 5642 | O | LYS | B | 564 | 18.154 | 8.166 | 86.275 | 1.00 | 25.84 | O |
| ATOM | 5643 | CB | LYS | B | 564 | 21.311 | 8.737 | 84.744 | 1.00 | 25.48 | C |
| ATOM | 5644 | CG | LYS | B | 564 | 21.201 | 7.532 | 83.815 | 1.00 | 25.63 | C |
| ATOM | 5645 | CD | LYS | B | 564 | 22.565 | 7.161 | 83.231 | 1.00 | 25.47 | C |
| ATOM | 5646 | CE | LYS | B | 564 | 22.575 | 5.754 | 82.640 | 1.00 | 25.59 | C |
| ATOM | 5647 | NZ | LYS | B | 564 | 21.694 | 5.626 | 81.451 | 1.00 | 26.26 | N |
| ATOM | 5648 | N | PHE | B | 565 | 20.147 | 8.102 | 87.323 | 1.00 | 25.97 | N |
| ATOM | 5649 | CA | PHE | B | 565 | 19.662 | 7.372 | 88.488 | 1.00 | 25.98 | C |
| ATOM | 5650 | C | PHE | B | 565 | 19.298 | 8.312 | 89.627 | 1.00 | 26.45 | C |
| ATOM | 5651 | O | PHE | B | 565 | 20.030 | 9.253 | 89.931 | 1.00 | 26.44 | O |
| ATOM | 5652 | CB | PHE | B | 565 | 20.722 | 6.368 | 88.955 | 1.00 | 25.91 | C |
| ATOM | 5653 | CG | PHE | B | 565 | 20.435 | 5.729 | 90.294 | 1.00 | 25.22 | C |
| ATOM | 5654 | CD1 | PHE | B | 565 | 19.303 | 4.932 | 90.484 | 1.00 | 24.74 | C |
| ATOM | 5655 | CD2 | PHE | B | 565 | 21.319 | 5.899 | 91.355 | 1.00 | 24.38 | C |
| ATOM | 5656 | CE1 | PHE | B | 565 | 19.052 | 4.329 | 91.725 | 1.00 | 24.75 | C |
| ATOM | 5657 | CE2 | PHE | B | 565 | 21.077 | 5.304 | 92.592 | 1.00 | 24.26 | C |
| ATOM | 5658 | CZ | PHE | B | 565 | 19.943 | 4.514 | 92.776 | 1.00 | 24.74 | C |
| ATOM | 5659 | N | LEU | B | 566 | 18.147 | 8.055 | 90.241 | 1.00 | 27.14 | N |
| ATOM | 5660 | CA | LEU | B | 566 | 17.809 | 8.649 | 91.529 | 1.00 | 27.66 | C |
| ATOM | 5661 | C | LEU | B | 566 | 17.271 | 7.565 | 92.452 | 1.00 | 28.17 | C |
| ATOM | 5662 | O | LEU | B | 566 | 16.233 | 6.950 | 92.177 | 1.00 | 28.23 | O |
| ATOM | 5663 | CB | LEU | B | 566 | 16.792 | 9.772 | 91.374 | 1.00 | 27.63 | C |
| ATOM | 5664 | CG | LEU | B | 566 | 16.215 | 10.342 | 92.669 | 1.00 | 27.70 | C |
| ATOM | 5665 | CD1 | LEU | B | 566 | 17.205 | 11.273 | 93.360 | 1.00 | 27.78 | C |
| ATOM | 5666 | CD2 | LEU | B | 566 | 14.918 | 11.061 | 92.365 | 1.00 | 28.28 | C |
| ATOM | 5667 | N | GLY | B | 567 | 17.990 | 7.334 | 93.544 | 1.00 | 28.66 | N |
| ATOM | 5668 | CA | GLY | B | 567 | 17.614 | 6.304 | 94.505 | 1.00 | 29.25 | C |
| ATOM | 5669 | C | GLY | B | 567 | 18.082 | 6.608 | 95.911 | 1.00 | 29.57 | C |
| ATOM | 5670 | O | GLY | B | 567 | 18.999 | 7.402 | 96.116 | 1.00 | 29.64 | O |
| ATOM | 5671 | N | VAL | B | 568 | 17.454 | 5.950 | 96.876 | 1.00 | 29.97 | N |
| ATOM | 5672 | CA | VAL | B | 568 | 17.665 | 6.243 | 98.291 | 1.00 | 30.48 | C |
| ATOM | 5673 | C | VAL | B | 568 | 18.974 | 5.681 | 98.837 | 1.00 | 30.86 | C |
| ATOM | 5674 | O | VAL | B | 568 | 19.497 | 4.687 | 98.329 | 1.00 | 30.82 | O |
| ATOM | 5675 | CB | VAL | B | 568 | 16.493 | 5.717 | 99.159 | 1.00 | 30.46 | C |
| ATOM | 5676 | CG1 | VAL | B | 568 | 15.225 | 6.531 | 98.917 | 1.00 | 30.37 | C |
| ATOM | 5677 | CG2 | VAL | B | 568 | 16.238 | 4.235 | 98.901 | 1.00 | 30.44 | C |
| ATOM | 5678 | N | GLY | B | 569 | 19.508 | 6.344 | 99.857 | 1.00 | 31.30 | N |
| ATOM | 5679 | CA | GLY | B | 569 | 20.507 | 5.729 | 100.723 | 1.00 | 32.08 | C |
| ATOM | 5680 | C | GLY | B | 569 | 19.716 | 4.937 | 101.750 | 1.00 | 32.52 | C |
| ATOM | 5681 | O | GLY | B | 569 | 19.173 | 5.527 | 102.682 | 1.00 | 32.58 | O |
| ATOM | 5682 | N | PRO | B | 570 | 19.627 | 3.602 | 101.578 | 1.00 | 32.83 | N |
| ATOM | 5683 | CA | PRO | B | 570 | 18.696 | 2.799 | 102.373 | 1.00 | 33.33 | C |
| ATOM | 5684 | C | PRO | B | 570 | 19.030 | 2.728 | 103.866 | 1.00 | 33.89 | C |
| ATOM | 5685 | O | PRO | B | 570 | 20.175 | 2.963 | 104.271 | 1.00 | 34.15 | O |
| ATOM | 5686 | CB | PRO | B | 570 | 18.798 | 1.413 | 101.741 | 1.00 | 33.23 | C |
| ATOM | 5687 | CG | PRO | B | 570 | 20.145 | 1.377 | 101.129 | 1.00 | 33.35 | C |
| ATOM | 5688 | CD | PRO | B | 570 | 20.405 | 2.768 | 100.645 | 1.00 | 32.95 | C |
| ATOM | 5689 | N | GLY | B | 571 | 18.017 | 2.409 | 104.665 | 1.00 | 34.24 | N |
| ATOM | 5690 | CA | GLY | B | 571 | 18.140 | 2.380 | 106.110 | 1.00 | 34.46 | C |
| ATOM | 5691 | C | GLY | B | 571 | 17.005 | 3.167 | 106.721 | 1.00 | 34.66 | C |
| ATOM | 5692 | O | GLY | B | 571 | 15.837 | 2.938 | 106.396 | 1.00 | 34.67 | O |
| ATOM | 5693 | N | THR | B | 572 | 17.349 | 4.110 | 107.591 | 1.00 | 34.85 | N |
| ATOM | 5694 | CA | THR | B | 572 | 16.346 | 4.870 | 108.330 | 1.00 | 35.16 | C |
| ATOM | 5695 | C | THR | B | 572 | 16.631 | 6.369 | 108.291 | 1.00 | 35.38 | C |
| ATOM | 5696 | O | THR | B | 572 | 17.777 | 6.801 | 108.461 | 1.00 | 35.30 | O |
| ATOM | 5697 | CB | THR | B | 572 | 16.231 | 4.363 | 109.791 | 1.00 | 35.21 | C |
| ATOM | 5698 | OG1 | THR | B | 572 | 15.976 | 2.950 | 109.780 | 1.00 | 34.70 | O |
| ATOM | 5699 | CG2 | THR | B | 572 | 15.106 | 5.077 | 110.543 | 1.00 | 35.28 | C |
| ATOM | 5700 | N | GLY | B | 573 | 15.579 | 7.150 | 108.054 | 1.00 | 35.58 | N |
| ATOM | 5701 | CA | GLY | B | 573 | 15.686 | 8.604 | 107.998 | 1.00 | 36.05 | C |
| ATOM | 5702 | C | GLY | B | 573 | 15.529 | 9.269 | 109.352 | 1.00 | 36.35 | C |
| ATOM | 5703 | O | GLY | B | 573 | 15.309 | 8.602 | 110.367 | 1.00 | 36.43 | O |
| ATOM | 5704 | N | ILE | B | 574 | 15.647 | 10.592 | 109.364 | 1.00 | 36.61 | N |
| ATOM | 5705 | CA | ILE | B | 574 | 15.446 | 11.366 | 110.580 | 1.00 | 36.94 | C |
| ATOM | 5706 | C | ILE | B | 574 | 14.426 | 12.472 | 110.368 | 1.00 | 37.18 | C |
| ATOM | 5707 | O | ILE | B | 574 | 14.027 | 12.760 | 109.241 | 1.00 | 37.34 | O |
| ATOM | 5708 | CB | ILE | B | 574 | 16.763 | 11.993 | 111.105 | 1.00 | 36.90 | C |
| ATOM | 5709 | CG1 | ILE | B | 574 | 17.313 | 13.025 | 110.111 | 1.00 | 37.13 | C |
| ATOM | 5710 | CG2 | ILE | B | 574 | 17.780 | 10.909 | 111.422 | 1.00 | 36.49 | C |
| ATOM | 5711 | CD1 | ILE | B | 574 | 18.160 | 14.118 | 110.751 | 1.00 | 37.46 | C |
| ATOM | 5712 | N | VAL | B | 575 | 14.002 | 13.076 | 111.472 | 1.00 | 37.49 | N |
| ATOM | 5713 | CA | VAL | B | 575 | 13.242 | 14.314 | 111.435 | 1.00 | 37.61 | C |
| ATOM | 5714 | C | VAL | B | 575 | 14.035 | 15.339 | 112.232 | 1.00 | 37.86 | C |
| ATOM | 5715 | O | VAL | B | 575 | 14.579 | 15.024 | 113.289 | 1.00 | 37.86 | O |
| ATOM | 5716 | CB | VAL | B | 575 | 11.823 | 14.147 | 112.020 | 1.00 | 37.43 | C |
| ATOM | 5717 | CG1 | VAL | B | 575 | 11.047 | 15.451 | 111.935 | 1.00 | 37.47 | C |
| ATOM | 5718 | CG2 | VAL | B | 575 | 11.076 | 13.050 | 111.291 | 1.00 | 37.45 | C |
| ATOM | 5719 | N | LEU | B | 576 | 14.116 | 16.555 | 111.706 | 1.00 | 38.30 | N |
| ATOM | 5720 | CA | LEU | B | 576 | 14.815 | 17.644 | 112.380 | 1.00 | 38.85 | C |
| ATOM | 5721 | C | LEU | B | 576 | 14.064 | 18.095 | 113.636 | 1.00 | 39.16 | C |
| ATOM | 5722 | O | LEU | B | 576 | 12.860 | 18.374 | 113.590 | 1.00 | 39.28 | O |
| ATOM | 5723 | CB | LEU | B | 576 | 15.035 | 18.817 | 111.413 | 1.00 | 38.80 | C |
| ATOM | 5724 | CG | LEU | B | 576 | 16.334 | 18.856 | 110.591 | 1.00 | 38.97 | C |
| ATOM | 5725 | CD1 | LEU | B | 576 | 16.779 | 17.485 | 110.064 | 1.00 | 38.94 | C |
| ATOM | 5726 | CD2 | LEU | B | 576 | 16.219 | 19.866 | 109.451 | 1.00 | 38.87 | C |
| ATOM | 5727 | N | ARG | B | 577 | 14.791 | 18.156 | 114.751 | 1.00 | 39.47 | N |
| ATOM | 5728 | CA | ARG | B | 577 | 14.214 | 18.464 | 116.067 | 1.00 | 39.57 | C |
| ATOM | 5729 | C | ARG | B | 577 | 14.683 | 19.818 | 116.616 | 1.00 | 39.85 | C |
| ATOM | 5730 | O | ARG | B | 577 | 14.152 | 20.300 | 117.613 | 1.00 | 40.02 | O |
| ATOM | 5731 | CB | ARG | B | 577 | 14.559 | 17.347 | 117.062 | 1.00 | 39.61 | C |
| ATOM | 5732 | CG | ARG | B | 577 | 16.057 | 17.160 | 117.235 | 1.00 | 39.34 | C |
| ATOM | 5733 | CD | ARG | B | 577 | 16.445 | 16.001 | 118.139 | 1.00 | 38.90 | C |
| ATOM | 5734 | NE | ARG | B | 577 | 17.856 | 16.123 | 118.501 | 1.00 | 37.10 | N |
| ATOM | 5735 | CZ | ARG | B | 577 | 18.877 | 15.747 | 117.732 | 1.00 | 36.86 | C |
| ATOM | 5736 | NH1 | ARG | B | 577 | 18.673 | 15.194 | 116.543 | 1.00 | 36.37 | N |
| ATOM | 5737 | NH2 | ARG | B | 577 | 20.117 | 15.928 | 118.158 | 1.00 | 37.21 | N |
| ATOM | 5738 | N | ASN | B | 578 | 15.684 | 20.410 | 115.965 | 1.00 | 40.06 | N |
| ATOM | 5739 | CA | ASN | B | 578 | 16.254 | 21.694 | 116.374 | 1.00 | 40.32 | C |
| ATOM | 5740 | C | ASN | B | 578 | 16.091 | 22.747 | 115.289 | 1.00 | 40.25 | C |
| ATOM | 5741 | O | ASN | B | 578 | 15.466 | 22.500 | 114.260 | 1.00 | 40.30 | O |
| ATOM | 5742 | CB | ASN | B | 578 | 17.752 | 21.553 | 116.676 | 1.00 | 40.38 | C |
| ATOM | 5743 | CG | ASN | B | 578 | 18.060 | 20.411 | 117.614 | 1.00 | 41.54 | C |
| ATOM | 5744 | OD1 | ASN | B | 578 | 18.634 | 19.400 | 117.204 | 1.00 | 42.10 | O |
| ATOM | 5745 | ND2 | ASN | B | 578 | 17.684 | 20.561 | 118.885 | 1.00 | 42.88 | N |
| ATOM | 5746 | N | GLY | B | 579 | 16.657 | 23.925 | 115.544 | 1.00 | 40.26 | N |
| ATOM | 5747 | CA | GLY | B | 579 | 16.872 | 24.945 | 114.526 | 1.00 | 40.10 | C |
| ATOM | 5748 | C | GLY | B | 579 | 15.645 | 25.481 | 113.817 | 1.00 | 40.14 | C |
| ATOM | 5749 | O | GLY | B | 579 | 14.511 | 25.128 | 114.164 | 1.00 | 40.10 | O |
| ATOM | 5750 | N | PRO | B | 580 | 15.867 | 26.339 | 112.803 | 1.00 | 40.07 | N |
| ATOM | 5751 | CA | PRO | B | 580 | 14.798 | 27.013 | 112.069 | 1.00 | 39.90 | C |
| ATOM | 5752 | C | PRO | B | 580 | 14.064 | 26.101 | 111.080 | 1.00 | 39.80 | C |
| ATOM | 5753 | O | PRO | B | 580 | 13.168 | 26.567 | 110.364 | 1.00 | 39.73 | O |
| ATOM | 5754 | CB | PRO | B | 580 | 15.534 | 28.135 | 111.317 | 1.00 | 39.81 | C |
| ATOM | 5755 | CG | PRO | B | 580 | 16.966 | 28.099 | 111.802 | 1.00 | 40.10 | C |
| ATOM | 5756 | CD | PRO | B | 580 | 17.195 | 26.719 | 112.293 | 1.00 | 40.07 | C |
| ATOM | 5757 | N | HIS | B | 581 | 14.442 | 24.822 | 111.043 | 1.00 | 39.64 | N |
| ATOM | 5758 | CA | HIS | B | 581 | 13.836 | 23.853 | 110.125 | 1.00 | 39.49 | C |
| ATOM | 5759 | C | HIS | B | 581 | 13.340 | 22.605 | 110.847 | 1.00 | 39.40 | C |
| ATOM | 5760 | O | HIS | B | 581 | 13.239 | 21.530 | 110.246 | 1.00 | 39.44 | O |
| ATOM | 5761 | CB | HIS | B | 581 | 14.815 | 23.476 | 109.004 | 1.00 | 39.61 | C |
| ATOM | 5762 | CG | HIS | B | 581 | 15.251 | 24.639 | 108.170 | 1.00 | 39.48 | C |
| ATOM | 5763 | ND1 | HIS | B | 581 | 14.450 | 25.196 | 107.197 | 1.00 | 39.59 | N |
| ATOM | 5764 | CD2 | HIS | B | 581 | 16.398 | 25.359 | 108.174 | 1.00 | 39.57 | C |
| ATOM | 5765 | CE1 | HIS | B | 581 | 15.084 | 26.210 | 106.635 | 1.00 | 39.71 | C |
| ATOM | 5766 | NE2 | HIS | B | 581 | 16.270 | 26.328 | 107.207 | 1.00 | 40.00 | N |
| ATOM | 5767 | N | LYS | B | 582 | 13.027 | 22.755 | 112.134 | 1.00 | 39.25 | N |
| ATOM | 5768 | CA | LYS | B | 582 | 12.456 | 21.669 | 112.927 | 1.00 | 39.08 | C |
| ATOM | 5769 | C | LYS | B | 582 | 11.193 | 21.146 | 112.248 | 1.00 | 38.75 | C |
| ATOM | 5770 | O | LYS | B | 582 | 10.345 | 21.929 | 111.818 | 1.00 | 38.80 | O |
| ATOM | 5771 | CB | LYS | B | 582 | 12.142 | 22.146 | 114.348 | 1.00 | 39.21 | C |
| ATOM | 5772 | CG | LYS | B | 582 | 11.474 | 21.093 | 115.223 | 1.00 | 39.76 | C |
| ATOM | 5773 | CD | LYS | B | 582 | 11.073 | 21.658 | 116.569 | 1.00 | 40.91 | C |
| ATOM | 5774 | CE | LYS | B | 582 | 10.199 | 20.677 | 117.345 | 1.00 | 40.90 | C |
| ATOM | 5775 | NZ | LYS | B | 582 | 9.701 | 21.292 | 118.608 | 1.00 | 41.14 | N |
| ATOM | 5776 | N | GLY | B | 583 | 11.089 | 19.825 | 112.141 | 1.00 | 38.32 | N |
| ATOM | 5777 | CA | GLY | B | 583 | 9.947 | 19.190 | 111.491 | 1.00 | 38.00 | C |
| ATOM | 5778 | C | GLY | B | 583 | 10.252 | 18.663 | 110.099 | 1.00 | 37.82 | C |
| ATOM | 5779 | O | GLY | B | 583 | 9.493 | 17.853 | 109.559 | 1.00 | 37.66 | O |
| ATOM | 5780 | N | ARG | B | 584 | 11.362 | 19.123 | 109.520 | 1.00 | 37.58 | N |
| ATOM | 5781 | CA | ARG | B | 584 | 11.797 | 18.685 | 108.194 | 1.00 | 37.34 | C |
| ATOM | 5782 | C | ARG | B | 584 | 12.222 | 17.218 | 108.219 | 1.00 | 37.32 | C |
| ATOM | 5783 | O | ARG | B | 584 | 13.001 | 16.802 | 109.080 | 1.00 | 37.48 | O |
| ATOM | 5784 | CB | ARG | B | 584 | 12.946 | 19.562 | 107.690 | 1.00 | 37.55 | C |
| ATOM | 5785 | CG | ARG | B | 584 | 13.509 | 19.178 | 106.320 | 1.00 | 37.16 | C |
| ATOM | 5786 | CD | ARG | B | 584 | 14.540 | 20.196 | 105.853 | 1.00 | 37.20 | C |
| ATOM | 5787 | NE | ARG | B | 584 | 13.916 | 21.424 | 105.358 | 1.00 | 36.40 | N |
| ATOM | 5788 | CZ | ARG | B | 584 | 14.566 | 22.557 | 105.099 | 1.00 | 35.56 | C |
| ATOM | 5789 | NH1 | ARG | B | 584 | 15.877 | 22.646 | 105.292 | 1.00 | 34.74 | N |
| ATOM | 5790 | NH2 | ARG | B | 584 | 13.897 | 23.609 | 104.645 | 1.00 | 34.98 | N |
| ATOM | 5791 | N | ILE | B | 585 | 11.698 | 16.448 | 107.269 | 1.00 | 36.94 | N |
| ATOM | 5792 | CA | ILE | B | 585 | 11.998 | 15.027 | 107.154 | 1.00 | 36.55 | C |
| ATOM | 5793 | C | ILE | B | 585 | 13.111 | 14.814 | 106.132 | 1.00 | 36.36 | C |
| ATOM | 5794 | O | ILE | B | 585 | 12.997 | 15.242 | 104.986 | 1.00 | 36.43 | O |
| ATOM | 5795 | CB | ILE | B | 585 | 10.746 | 14.220 | 106.755 | 1.00 | 36.45 | C |
| ATOM | 5796 | CG1 | ILE | B | 585 | 9.583 | 14.554 | 107.694 | 1.00 | 36.12 | C |
| ATOM | 5797 | CG2 | ILE | B | 585 | 11.052 | 12.731 | 106.769 | 1.00 | 36.33 | C |
| ATOM | 5798 | CD1 | ILE | B | 585 | 8.226 | 14.168 | 107.161 | 1.00 | 36.54 | C |
| ATOM | 5799 | N | LEU | B | 586 | 14.180 | 14.149 | 106.560 | 1.00 | 36.05 | N |
| ATOM | 5800 | CA | LEU | B | 586 | 15.366 | 13.948 | 105.728 | 1.00 | 35.62 | C |
| ATOM | 5801 | C | LEU | B | 586 | 15.514 | 12.509 | 105.230 | 1.00 | 35.08 | C |
| ATOM | 5802 | O | LEU | B | 586 | 15.666 | 11.579 | 106.024 | 1.00 | 34.98 | O |
| ATOM | 5803 | CB | LEU | B | 586 | 16.630 | 14.392 | 106.482 | 1.00 | 35.81 | C |
| ATOM | 5804 | CG | LEU | B | 586 | 17.278 | 15.743 | 106.137 | 1.00 | 36.16 | C |
| ATOM | 5805 | CD1 | LEU | B | 586 | 16.293 | 16.904 | 106.136 | 1.00 | 36.88 | C |
| ATOM | 5806 | CD2 | LEU | B | 586 | 18.431 | 16.039 | 107.073 | 1.00 | 35.80 | C |
| ATOM | 5807 | N | ILE | B | 587 | 15.459 | 12.351 | 103.907 | 1.00 | 34.31 | N |
| ATOM | 5808 | CA | ILE | B | 587 | 15.686 | 11.074 | 103.237 | 1.00 | 33.55 | C |
| ATOM | 5809 | C | ILE | B | 587 | 16.980 | 11.154 | 102.422 | 1.00 | 33.01 | C |
| ATOM | 5810 | O | ILE | B | 587 | 17.060 | 11.920 | 101.458 | 1.00 | 33.09 | O |
| ATOM | 5811 | CB | ILE | B | 587 | 14.506 | 10.698 | 102.294 | 1.00 | 33.56 | C |
| ATOM | 5812 | CG1 | ILE | B | 587 | 13.152 | 10.863 | 103.002 | 1.00 | 33.79 | C |
| ATOM | 5813 | CG2 | ILE | B | 587 | 14.684 | 9.289 | 101.715 | 1.00 | 33.31 | C |
| ATOM | 5814 | CD1 | ILE | B | 587 | 12.878 | 9.860 | 104.120 | 1.00 | 33.62 | C |
| ATOM | 5815 | N | PRO | B | 588 | 18.001 | 10.373 | 102.814 | 1.00 | 32.39 | N |
| ATOM | 5816 | CA | PRO | B | 588 | 19.259 | 10.302 | 102.071 | 1.00 | 31.76 | C |
| ATOM | 5817 | C | PRO | B | 588 | 19.070 | 9.642 | 100.712 | 1.00 | 31.10 | C |
| ATOM | 5818 | O | PRO | B | 588 | 18.436 | 8.589 | 100.619 | 1.00 | 31.22 | O |
| ATOM | 5819 | CB | PRO | B | 588 | 20.137 | 9.413 | 102.956 | 1.00 | 31.68 | C |
| ATOM | 5820 | CG | PRO | B | 588 | 19.491 | 9.452 | 104.306 | 1.00 | 31.90 | C |
| ATOM | 5821 | CD | PRO | B | 588 | 18.038 | 9.515 | 104.009 | 1.00 | 32.36 | C |
| ATOM | 5822 | N | VAL | B | 589 | 19.609 | 10.268 | 99.670 | 1.00 | 30.21 | N |
| ATOM | 5823 | CA | VAL | B | 589 | 19.554 | 9.719 | 98.313 | 1.00 | 29.28 | C |
| ATOM | 5824 | C | VAL | B | 589 | 20.881 | 9.947 | 97.587 | 1.00 | 28.85 | C |
| ATOM | 5825 | O | VAL | B | 589 | 21.765 | 10.654 | 98.085 | 1.00 | 28.63 | O |
| ATOM | 5826 | CB | VAL | B | 589 | 18.374 | 10.311 | 97.456 | 1.00 | 29.24 | C |
| ATOM | 5827 | CG1 | VAL | B | 589 | 17.014 | 9.908 | 98.009 | 1.00 | 29.04 | C |
| ATOM | 5828 | CG2 | VAL | B | 589 | 18.472 | 11.830 | 97.323 | 1.00 | 29.02 | C |
| ATOM | 5829 | N | TYR | B | 590 | 21.014 | 9.340 | 96.413 | 1.00 | 28.16 | N |
| ATOM | 5830 | CA | TYR | B | 590 | 22.165 | 9.592 | 95.559 | 1.00 | 27.74 | C |
| ATOM | 5831 | C | TYR | B | 590 | 21.829 | 9.522 | 94.070 | 1.00 | 27.39 | C |
| ATOM | 5832 | O | TYR | B | 590 | 20.942 | 8.770 | 93.653 | 1.00 | 27.57 | O |
| ATOM | 5833 | CB | TYR | B | 590 | 23.359 | 8.705 | 95.937 | 1.00 | 27.74 | C |
| ATOM | 5834 | CG | TYR | B | 590 | 23.156 | 7.221 | 95.777 | 1.00 | 27.61 | C |
| ATOM | 5835 | CD1 | TYR | B | 590 | 22.175 | 6.543 | 96.500 | 1.00 | 27.56 | C |
| ATOM | 5836 | CD2 | TYR | B | 590 | 23.974 | 6.484 | 94.927 | 1.00 | 27.72 | C |
| ATOM | 5837 | CE1 | TYR | B | 590 | 21.998 | 5.167 | 96.356 | 1.00 | 27.93 | C |
| ATOM | 5838 | CE2 | TYR | B | 590 | 23.811 | 5.108 | 94.783 | 1.00 | 28.39 | C |
| ATOM | 5839 | CZ | TYR | B | 590 | 22.820 | 4.457 | 95.500 | 1.00 | 27.82 | C |
| ATOM | 5840 | OH | TYR | B | 590 | 22.655 | 3.098 | 95.354 | 1.00 | 28.09 | O |
| ATOM | 5841 | N | THR | B | 591 | 22.538 | 10.324 | 93.282 | 1.00 | 26.90 | N |
| ATOM | 5842 | CA | THR | B | 591 | 22.256 | 10.461 | 91.857 | 1.00 | 26.48 | C |
| ATOM | 5843 | C | THR | B | 591 | 23.436 | 10.024 | 91.007 | 1.00 | 26.34 | C |
| ATOM | 5844 | O | THR | B | 591 | 24.580 | 10.046 | 91.467 | 1.00 | 26.34 | O |
| ATOM | 5845 | CB | THR | B | 591 | 21.961 | 11.923 | 91.491 | 1.00 | 26.30 | C |
| ATOM | 5846 | OG1 | THR | B | 591 | 23.095 | 12.728 | 91.836 | 1.00 | 26.15 | O |
| ATOM | 5847 | CG2 | THR | B | 591 | 20.720 | 12.433 | 92.225 | 1.00 | 26.33 | C |
| ATOM | 5848 | N | THR | B | 592 | 23.146 | 9.626 | 89.770 | 1.00 | 26.01 | N |
| ATOM | 5849 | CA | THR | B | 592 | 24.163 | 9.540 | 88.730 | 1.00 | 26.13 | C |
| ATOM | 5850 | C | THR | B | 592 | 23.840 | 10.551 | 87.621 | 1.00 | 26.25 | C |
| ATOM | 5851 | O | THR | B | 592 | 22.731 | 11.100 | 87.579 | 1.00 | 26.39 | O |
| ATOM | 5852 | CB | THR | B | 592 | 24.276 | 8.127 | 88.105 | 1.00 | 26.07 | C |
| ATOM | 5853 | OG1 | THR | B | 592 | 23.108 | 7.849 | 87.327 | 1.00 | 25.95 | O |
| ATOM | 5854 | CG2 | THR | B | 592 | 24.467 | 7.047 | 89.173 | 1.00 | 26.68 | C |
| ATOM | 5855 | N | ASN | B | 593 | 24.813 | 10.797 | 86.743 | 1.00 | 26.05 | N |
| ATOM | 5856 | CA | ASN | B | 593 | 24.621 | 11.643 | 85.575 | 1.00 | 25.99 | C |
| ATOM | 5857 | C | ASN | B | 593 | 24.926 | 10.865 | 84.309 | 1.00 | 26.11 | C |
| ATOM | 5858 | O | ASN | B | 593 | 25.407 | 9.738 | 84.376 | 1.00 | 26.40 | O |
| ATOM | 5859 | CB | ASN | B | 593 | 25.457 | 12.934 | 85.664 | 1.00 | 26.00 | C |
| ATOM | 5860 | CG | ASN | B | 593 | 26.961 | 12.702 | 85.496 | 1.00 | 25.94 | C |
| ATOM | 5861 | OD1 | ASN | B | 593 | 27.408 | 11.822 | 84.752 | 1.00 | 25.81 | O |
| ATOM | 5862 | ND2 | ASN | B | 593 | 27.749 | 13.521 | 86.177 | 1.00 | 26.36 | N |
| ATOM | 5863 | N | ASN | B | 594 | 24.651 | 11.471 | 83.162 | 1.00 | 26.38 | N |
| ATOM | 5864 | CA | ASN | B | 594 | 24.794 | 10.800 | 81.871 | 1.00 | 26.51 | C |
| ATOM | 5865 | C | ASN | B | 594 | 26.202 | 10.864 | 81.306 | 1.00 | 26.61 | C |
| ATOM | 5866 | O | ASN | B | 594 | 26.502 | 10.207 | 80.305 | 1.00 | 26.57 | O |
| ATOM | 5867 | CB | ASN | B | 594 | 23.792 | 11.370 | 80.865 | 1.00 | 26.64 | C |
| ATOM | 5868 | CG | ASN | B | 594 | 22.373 | 10.941 | 81.161 | 1.00 | 26.66 | C |
| ATOM | 5869 | OD1 | ASN | B | 594 | 22.070 | 9.746 | 81.177 | 1.00 | 28.18 | O |
| ATOM | 5870 | ND2 | ASN | B | 594 | 21.492 | 11.908 | 81.388 | 1.00 | 25.44 | N |
| ATOM | 5871 | N | VAL | B | 595 | 27.061 | 11.656 | 81.948 | 1.00 | 26.75 | N |
| ATOM | 5872 | CA | VAL | B | 595 | 28.459 | 11.766 | 81.536 | 1.00 | 26.87 | C |
| ATOM | 5873 | C | VAL | B | 595 | 29.241 | 10.506 | 81.914 | 1.00 | 26.95 | C |
| ATOM | 5874 | O | VAL | B | 595 | 29.977 | 9.973 | 81.085 | 1.00 | 27.11 | O |
| ATOM | 5875 | CB | VAL | B | 595 | 29.153 | 13.036 | 82.115 | 1.00 | 26.95 | C |
| ATOM | 5876 | CG1 | VAL | B | 595 | 30.642 | 13.078 | 81.743 | 1.00 | 26.70 | C |
| ATOM | 5877 | CG2 | VAL | B | 595 | 28.452 | 14.298 | 81.632 | 1.00 | 26.93 | C |
| ATOM | 5878 | N | SER | B | 596 | 29.077 | 10.032 | 83.151 | 1.00 | 26.91 | N |
| ATOM | 5879 | CA | SER | B | 596 | 29.875 | 8.895 | 83.647 | 1.00 | 26.87 | C |
| ATOM | 5880 | C | SER | B | 596 | 29.094 | 7.830 | 84.424 | 1.00 | 26.76 | C |
| ATOM | 5881 | O | SER | B | 596 | 29.647 | 6.769 | 84.747 | 1.00 | 26.56 | O |
| ATOM | 5882 | CB | SER | B | 596 | 31.067 | 9.386 | 84.477 | 1.00 | 26.82 | C |
| ATOM | 5883 | OG | SER | B | 596 | 30.652 | 10.117 | 85.619 | 1.00 | 27.17 | O |
| ATOM | 5884 | N | HIS | B | 597 | 27.823 | 8.116 | 84.714 | 1.00 | 26.59 | N |
| ATOM | 5885 | CA | HIS | B | 597 | 26.920 | 7.166 | 85.378 | 1.00 | 26.64 | C |
| ATOM | 5886 | C | HIS | B | 597 | 27.582 | 6.478 | 86.591 | 1.00 | 26.73 | C |
| ATOM | 5887 | O | HIS | B | 597 | 28.069 | 7.162 | 87.490 | 1.00 | 26.88 | O |
| ATOM | 5888 | CB | HIS | B | 597 | 26.340 | 6.173 | 84.354 | 1.00 | 26.55 | C |
| ATOM | 5889 | CG | HIS | B | 597 | 25.268 | 5.280 | 84.899 | 1.00 | 26.12 | C |
| ATOM | 5890 | ND1 | HIS | B | 597 | 24.331 | 5.709 | 85.816 | 1.00 | 26.34 | N |
| ATOM | 5891 | CD2 | HIS | B | 597 | 24.977 | 3.983 | 84.643 | 1.00 | 26.46 | C |
| ATOM | 5892 | CE1 | HIS | B | 597 | 23.519 | 4.709 | 86.114 | 1.00 | 26.28 | C |
| ATOM | 5893 | NE2 | HIS | B | 597 | 23.886 | 3.651 | 85.413 | 1.00 | 26.62 | N |
| ATOM | 5894 | N | LEU | B | 598 | 27.633 | 5.150 | 86.605 | 1.00 | 26.92 | N |
| ATOM | 5895 | CA | LEU | B | 598 | 28.153 | 4.419 | 87.764 | 1.00 | 27.20 | C |
| ATOM | 5896 | C | LEU | B | 598 | 29.659 | 4.562 | 87.977 | 1.00 | 27.30 | C |
| ATOM | 5897 | O | LEU | B | 598 | 30.142 | 4.434 | 89.100 | 1.00 | 27.52 | O |
| ATOM | 5898 | CB | LEU | B | 598 | 27.760 | 2.936 | 87.702 | 1.00 | 27.08 | C |
| ATOM | 5899 | CG | LEU | B | 598 | 26.335 | 2.580 | 88.153 | 1.00 | 27.37 | C |
| ATOM | 5900 | CD1 | LEU | B | 598 | 26.156 | 1.068 | 88.243 | 1.00 | 26.86 | C |
| ATOM | 5901 | CD2 | LEU | B | 598 | 25.973 | 3.235 | 89.495 | 1.00 | 27.20 | C |
| ATOM | 5902 | N | ASN | B | 599 | 30.382 | 4.853 | 86.900 | 1.00 | 27.41 | N |
| ATOM | 5903 | CA | ASN | B | 599 | 31.841 | 4.907 | 86.916 | 1.00 | 27.26 | C |
| ATOM | 5904 | C | ASN | B | 599 | 32.458 | 6.139 | 87.572 | 1.00 | 27.24 | C |
| ATOM | 5905 | O | ASN | B | 599 | 33.580 | 6.057 | 88.072 | 1.00 | 27.48 | O |
| ATOM | 5906 | CB | ASN | B | 599 | 32.387 | 4.755 | 85.493 | 1.00 | 27.40 | C |
| ATOM | 5907 | CG | ASN | B | 599 | 31.937 | 3.461 | 84.834 | 1.00 | 27.46 | C |
| ATOM | 5908 | OD1 | ASN | B | 599 | 31.794 | 2.429 | 85.492 | 1.00 | 28.03 | O |
| ATOM | 5909 | ND2 | ASN | B | 599 | 31.716 | 3.511 | 83.530 | 1.00 | 27.49 | N |
| ATOM | 5910 | N | GLY | B | 600 | 31.748 | 7.271 | 87.567 | 1.00 | 26.96 | N |
| ATOM | 5911 | CA | GLY | B | 600 | 32.295 | 8.508 | 88.137 | 1.00 | 26.57 | C |
| ATOM | 5912 | C | GLY | B | 600 | 31.358 | 9.616 | 88.609 | 1.00 | 26.50 | C |
| ATOM | 5913 | O | GLY | B | 600 | 31.808 | 10.744 | 88.832 | 1.00 | 26.38 | O |
| ATOM | 5914 | N | SER | B | 601 | 30.071 | 9.314 | 88.792 | 1.00 | 26.34 | N |
| ATOM | 5915 | CA | SER | B | 601 | 29.101 | 10.375 | 89.106 | 1.00 | 26.22 | C |
| ATOM | 5916 | C | SER | B | 601 | 28.231 | 10.209 | 90.363 | 1.00 | 25.97 | C |
| ATOM | 5917 | O | SER | B | 601 | 27.520 | 11.139 | 90.741 | 1.00 | 25.72 | O |
| ATOM | 5918 | CB | SER | B | 601 | 28.205 | 10.652 | 87.896 | 1.00 | 26.05 | C |
| ATOM | 5919 | OG | SER | B | 601 | 27.281 | 9.604 | 87.718 | 1.00 | 25.94 | O |
| ATOM | 5920 | N | GLN | B | 602 | 28.275 | 9.042 | 90.997 | 1.00 | 25.94 | N |
| ATOM | 5921 | CA | GLN | B | 602 | 27.492 | 8.810 | 92.215 | 1.00 | 26.00 | C |
| ATOM | 5922 | C | GLN | B | 602 | 27.681 | 9.944 | 93.229 | 1.00 | 25.96 | C |
| ATOM | 5923 | O | GLN | B | 602 | 28.796 | 10.187 | 93.710 | 1.00 | 25.50 | O |
| ATOM | 5924 | CB | GLN | B | 602 | 27.811 | 7.443 | 92.816 | 1.00 | 25.87 | C |
| ATOM | 5925 | CG | GLN | B | 602 | 27.200 | 6.294 | 92.034 | 1.00 | 26.76 | C |
| ATOM | 5926 | CD | GLN | B | 602 | 27.801 | 4.941 | 92.378 | 1.00 | 28.33 | C |
| ATOM | 5927 | OE1 | GLN | B | 602 | 27.131 | 4.082 | 92.944 | 1.00 | 28.47 | O |
| ATOM | 5928 | NE2 | GLN | B | 602 | 29.069 | 4.743 | 92.023 | 1.00 | 28.92 | N |
| ATOM | 5929 | N | SER | B | 603 | 26.581 | 10.651 | 93.507 | 1.00 | 25.99 | N |
| ATOM | 5930 | CA | SER | B | 603 | 26.598 | 11.867 | 94.317 | 1.00 | 26.03 | C |
| ATOM | 5931 | C | SER | B | 603 | 25.506 | 11.870 | 95.395 | 1.00 | 26.61 | C |
| ATOM | 5932 | O | SER | B | 603 | 24.310 | 11.798 | 95.089 | 1.00 | 26.42 | O |
| ATOM | 5933 | CB | SER | B | 603 | 26.452 | 13.095 | 93.424 | 1.00 | 25.94 | C |
| ATOM | 5934 | OG | SER | B | 603 | 27.558 | 13.236 | 92.550 | 1.00 | 25.81 | O |
| ATOM | 5935 | N | SER | B | 604 | 25.936 | 11.963 | 96.654 | 1.00 | 26.84 | N |
| ATOM | 5936 | CA | SER | B | 604 | 25.036 | 11.939 | 97.799 | 1.00 | 27.26 | C |
| ATOM | 5937 | C | SER | B | 604 | 24.345 | 13.280 | 97.980 | 1.00 | 27.43 | C |
| ATOM | 5938 | O | SER | B | 604 | 24.959 | 14.336 | 97.822 | 1.00 | 27.52 | O |
| ATOM | 5939 | CB | SER | B | 604 | 25.798 | 11.597 | 99.082 | 1.00 | 27.31 | C |
| ATOM | 5940 | OG | SER | B | 604 | 26.301 | 10.270 | 99.062 | 1.00 | 27.63 | O |
| ATOM | 5941 | N | ARG | B | 605 | 23.057 | 13.216 | 98.297 | 1.00 | 27.67 | N |
| ATOM | 5942 | CA | ARG | B | 605 | 22.272 | 14.380 | 98.709 | 1.00 | 27.87 | C |
| ATOM | 5943 | C | ARG | B | 605 | 21.086 | 13.879 | 99.536 | 1.00 | 27.96 | C |
| ATOM | 5944 | O | ARG | B | 605 | 20.961 | 12.683 | 99.791 | 1.00 | 27.61 | O |
| ATOM | 5945 | CB | ARG | B | 605 | 21.805 | 15.205 | 97.497 | 1.00 | 27.65 | C |
| ATOM | 5946 | CG | ARG | B | 605 | 20.676 | 14.572 | 96.681 | 1.00 | 28.18 | C |
| ATOM | 5947 | CD | ARG | B | 605 | 20.193 | 15.484 | 95.559 | 1.00 | 27.65 | C |
| ATOM | 5948 | NE | ARG | B | 605 | 21.177 | 15.604 | 94.486 | 1.00 | 27.34 | N |
| ATOM | 5949 | CZ | ARG | B | 605 | 21.264 | 16.639 | 93.651 | 1.00 | 27.57 | C |
| ATOM | 5950 | NH1 | ARG | B | 605 | 20.425 | 17.666 | 93.747 | 1.00 | 26.48 | N |
| ATOM | 5951 | NH2 | ARG | B | 605 | 22.196 | 16.643 | 92.708 | 1.00 | 27.95 | N |
| ATOM | 5952 | N | ILE | B | 606 | 20.229 | 14.792 | 99.971 | 1.00 | 28.51 | N |
| ATOM | 5953 | CA | ILE | B | 606 | 18.978 | 14.388 | 100.601 | 1.00 | 29.16 | C |
| ATOM | 5954 | C | ILE | B | 606 | 17.782 | 14.954 | 99.853 | 1.00 | 29.61 | C |
| ATOM | 5955 | O | ILE | B | 606 | 17.911 | 15.918 | 99.093 | 1.00 | 29.76 | O |
| ATOM | 5956 | CB | ILE | B | 606 | 18.902 | 14.769 | 102.112 | 1.00 | 29.06 | C |
| ATOM | 5957 | CG1 | ILE | B | 606 | 19.113 | 16.273 | 102.334 | 1.00 | 29.14 | C |
| ATOM | 5958 | CG2 | ILE | B | 606 | 19.900 | 13.949 | 102.928 | 1.00 | 29.31 | C |
| ATOM | 5959 | CD1 | ILE | B | 606 | 17.850 | 17.113 | 102.226 | 1.00 | 28.96 | C |
| ATOM | 5960 | N | ILE | B | 607 | 16.630 | 14.323 | 100.052 | 1.00 | 30.18 | N |
| ATOM | 5961 | CA | ILE | B | 607 | 15.349 | 14.933 | 99.723 | 1.00 | 30.64 | C |
| ATOM | 5962 | C | ILE | B | 607 | 14.627 | 15.161 | 101.043 | 1.00 | 30.96 | C |
| ATOM | 5963 | O | ILE | B | 607 | 14.878 | 14.451 | 102.020 | 1.00 | 30.90 | O |
| ATOM | 5964 | CB | ILE | B | 607 | 14.514 | 14.080 | 98.737 | 1.00 | 30.78 | C |
| ATOM | 5965 | CG1 | ILE | B | 607 | 14.104 | 12.739 | 99.371 | 1.00 | 30.89 | C |
| ATOM | 5966 | CG2 | ILE | B | 607 | 15.294 | 13.884 | 97.419 | 1.00 | 30.69 | C |
| ATOM | 5967 | CD1 | ILE | B | 607 | 13.109 | 11.912 | 98.558 | 1.00 | 30.47 | C |
| ATOM | 5968 | N | TYR | B | 608 | 13.752 | 16.159 | 101.080 | 1.00 | 31.45 | N |
| ATOM | 5969 | CA | TYR | B | 608 | 13.149 | 16.587 | 102.335 | 1.00 | 31.80 | C |
| ATOM | 5970 | C | TYR | B | 608 | 11.688 | 16.996 | 102.201 | 1.00 | 32.17 | C |
| ATOM | 5971 | O | TYR | B | 608 | 11.207 | 17.271 | 101.101 | 1.00 | 32.22 | O |
| ATOM | 5972 | CB | TYR | B | 608 | 13.964 | 17.728 | 102.949 | 1.00 | 31.85 | C |
| ATOM | 5973 | CG | TYR | B | 608 | 14.000 | 18.994 | 102.123 | 1.00 | 31.75 | C |
| ATOM | 5974 | CD1 | TYR | B | 608 | 14.921 | 19.145 | 101.090 | 1.00 | 32.30 | C |
| ATOM | 5975 | CD2 | TYR | B | 608 | 13.122 | 20.047 | 102.383 | 1.00 | 31.80 | C |
| ATOM | 5976 | CE1 | TYR | B | 608 | 14.964 | 20.305 | 100.324 | 1.00 | 32.00 | C |
| ATOM | 5977 | CE2 | TYR | B | 608 | 13.154 | 21.217 | 101.624 | 1.00 | 31.68 | C |
| ATOM | 5978 | CZ | TYR | B | 608 | 14.084 | 21.338 | 100.599 | 1.00 | 32.12 | C |
| ATOM | 5979 | OH | TYR | B | 608 | 14.138 | 22.484 | 99.838 | 1.00 | 32.33 | O |
| ATOM | 5980 | N | SER | B | 609 | 10.990 | 17.023 | 103.333 | 1.00 | 32.54 | N |
| ATOM | 5981 | CA | SER | B | 609 | 9.601 | 17.467 | 103.380 | 1.00 | 32.95 | C |
| ATOM | 5982 | C | SER | B | 609 | 9.363 | 18.375 | 104.574 | 1.00 | 33.27 | C |
| ATOM | 5983 | O | SER | B | 609 | 9.672 | 18.016 | 105.716 | 1.00 | 33.38 | O |
| ATOM | 5984 | CB | SER | B | 609 | 8.642 | 16.282 | 103.432 | 1.00 | 32.84 | C |
| ATOM | 5985 | OG | SER | B | 609 | 7.305 | 16.743 | 103.396 | 1.00 | 33.16 | O |
| ATOM | 5986 | N | ASP | B | 610 | 8.820 | 19.555 | 104.291 | 1.00 | 33.58 | N |
| ATOM | 5987 | CA | ASP | B | 610 | 8.516 | 20.548 | 105.309 | 1.00 | 33.99 | C |
| ATOM | 5988 | C | ASP | B | 610 | 7.012 | 20.598 | 105.614 | 1.00 | 34.18 | C |
| ATOM | 5989 | O | ASP | B | 610 | 6.564 | 21.409 | 106.426 | 1.00 | 34.26 | O |
| ATOM | 5990 | CB | ASP | B | 610 | 9.019 | 21.926 | 104.862 | 1.00 | 34.02 | C |
| ATOM | 5991 | CG | ASP | B | 610 | 10.538 | 22.052 | 104.918 | 1.00 | 34.47 | C |
| ATOM | 5992 | OD1 | ASP | B | 610 | 11.165 | 21.470 | 105.826 | 1.00 | 34.96 | O |
| ATOM | 5993 | OD2 | ASP | B | 610 | 11.108 | 22.760 | 104.060 | 1.00 | 34.58 | O |
| ATOM | 5994 | N | ASP | B | 611 | 6.235 | 19.735 | 104.964 | 1.00 | 34.49 | N |
| ATOM | 5995 | CA | ASP | B | 611 | 4.785 | 19.700 | 105.187 | 1.00 | 34.78 | C |
| ATOM | 5996 | C | ASP | B | 611 | 4.257 | 18.304 | 105.546 | 1.00 | 35.01 | C |
| ATOM | 5997 | O | ASP | B | 611 | 3.155 | 17.915 | 105.147 | 1.00 | 35.21 | O |
| ATOM | 5998 | CB | ASP | B | 611 | 4.018 | 20.329 | 104.008 | 1.00 | 34.62 | C |
| ATOM | 5999 | CG | ASP | B | 611 | 4.292 | 19.644 | 102.671 | 1.00 | 34.72 | C |
| ATOM | 6000 | OD1 | ASP | B | 611 | 5.063 | 18.656 | 102.615 | 1.00 | 34.47 | O |
| ATOM | 6001 | OD2 | ASP | B | 611 | 3.714 | 20.106 | 101.661 | 1.00 | 34.96 | O |
| ATOM | 6002 | N | HIS | B | 612 | 5.055 | 17.570 | 106.320 | 1.00 | 35.40 | N |
| ATOM | 6003 | CA | HIS | B | 612 | 4.690 | 16.247 | 106.841 | 1.00 | 35.62 | C |
| ATOM | 6004 | C | HIS | B | 612 | 4.533 | 15.207 | 105.736 | 1.00 | 35.75 | C |
| ATOM | 6005 | O | HIS | B | 612 | 3.606 | 14.393 | 105.760 | 1.00 | 35.77 | O |
| ATOM | 6006 | CB | HIS | B | 612 | 3.432 | 16.323 | 107.731 | 1.00 | 35.71 | C |
| ATOM | 6007 | CG | HIS | B | 612 | 3.451 | 17.462 | 108.705 | 1.00 | 35.47 | C |
| ATOM | 6008 | ND1 | HIS | B | 612 | 4.215 | 17.448 | 109.853 | 1.00 | 35.22 | N |
| ATOM | 6009 | CD2 | HIS | B | 612 | 2.821 | 18.662 | 108.687 | 1.00 | 35.10 | C |
| ATOM | 6010 | CE1 | HIS | B | 612 | 4.047 | 18.586 | 110.504 | 1.00 | 34.91 | C |
| ATOM | 6011 | NE2 | HIS | B | 612 | 3.206 | 19.339 | 109.818 | 1.00 | 34.78 | N |
| ATOM | 6012 | N | GLY | B | 613 | 5.445 | 15.249 | 104.765 | 1.00 | 35.94 | N |
| ATOM | 6013 | CA | GLY | B | 613 | 5.538 | 14.207 | 103.739 | 1.00 | 36.05 | C |
| ATOM | 6014 | C | GLY | B | 613 | 4.575 | 14.338 | 102.574 | 1.00 | 36.36 | C |
| ATOM | 6015 | O | GLY | B | 613 | 4.450 | 13.416 | 101.767 | 1.00 | 36.22 | O |
| ATOM | 6016 | N | LYS | B | 614 | 3.890 | 15.475 | 102.481 | 1.00 | 36.65 | N |
| ATOM | 6017 | CA | LYS | B | 614 | 3.011 | 15.724 | 101.346 | 1.00 | 36.99 | C |
| ATOM | 6018 | C | LYS | B | 614 | 3.830 | 16.029 | 100.092 | 1.00 | 36.76 | C |
| ATOM | 6019 | O | LYS | B | 614 | 3.769 | 15.287 | 99.119 | 1.00 | 36.98 | O |
| ATOM | 6020 | CB | LYS | B | 614 | 2.022 | 16.857 | 101.638 | 1.00 | 37.22 | C |
| ATOM | 6021 | CG | LYS | B | 614 | 0.857 | 16.887 | 100.663 | 1.00 | 37.79 | C |
| ATOM | 6022 | CD | LYS | B | 614 | 0.258 | 18.271 | 100.555 | 1.00 | 39.70 | C |
| ATOM | 6023 | CE | LYS | B | 614 | −0.439 | 18.449 | 99.213 | 1.00 | 40.53 | C |
| ATOM | 6024 | NZ | LYS | B | 614 | −1.048 | 19.803 | 99.106 | 1.00 | 41.84 | N |
| ATOM | 6025 | N | THR | B | 615 | 4.589 | 17.121 | 100.124 | 1.00 | 36.69 | N |
| ATOM | 6026 | CA | THR | B | 615 | 5.458 | 17.499 | 99.008 | 1.00 | 36.38 | C |
| ATOM | 6027 | C | THR | B | 615 | 6.920 | 17.272 | 99.377 | 1.00 | 36.22 | C |
| ATOM | 6028 | O | THR | B | 615 | 7.290 | 17.322 | 100.552 | 1.00 | 36.21 | O |
| ATOM | 6029 | CB | THR | B | 615 | 5.265 | 18.975 | 98.580 | 1.00 | 36.40 | C |
| ATOM | 6030 | OG1 | THR | B | 615 | 5.552 | 19.844 | 99.682 | 1.00 | 36.52 | O |
| ATOM | 6031 | CG2 | THR | B | 615 | 3.843 | 19.231 | 98.090 | 1.00 | 36.66 | C |
| ATOM | 6032 | N | TRP | B | 616 | 7.740 | 17.014 | 98.363 | 1.00 | 35.93 | N |
| ATOM | 6033 | CA | TRP | B | 616 | 9.162 | 16.776 | 98.553 | 1.00 | 35.60 | C |
| ATOM | 6034 | C | TRP | B | 616 | 9.999 | 17.710 | 97.682 | 1.00 | 35.51 | C |
| ATOM | 6035 | O | TRP | B | 616 | 9.561 | 18.135 | 96.607 | 1.00 | 35.66 | O |
| ATOM | 6036 | CB | TRP | B | 616 | 9.504 | 15.313 | 98.251 | 1.00 | 35.56 | C |
| ATOM | 6037 | CG | TRP | B | 616 | 8.823 | 14.360 | 99.178 | 1.00 | 35.55 | C |
| ATOM | 6038 | CD1 | TRP | B | 616 | 7.589 | 13.799 | 99.013 | 1.00 | 35.50 | C |
| ATOM | 6039 | CD2 | TRP | B | 616 | 9.325 | 13.869 | 100.427 | 1.00 | 35.18 | C |
| ATOM | 6040 | NE1 | TRP | B | 616 | 7.294 | 12.984 | 100.079 | 1.00 | 35.41 | N |
| ATOM | 6041 | CE2 | TRP | B | 616 | 8.340 | 13.007 | 100.962 | 1.00 | 35.03 | C |
| ATOM | 6042 | CE3 | TRP | B | 616 | 10.511 | 14.068 | 101.143 | 1.00 | 35.17 | C |
| ATOM | 6043 | CZ2 | TRP | B | 616 | 8.503 | 12.346 | 102.183 | 1.00 | 34.96 | C |
| ATOM | 6044 | CZ3 | TRP | B | 616 | 10.672 | 13.412 | 102.364 | 1.00 | 35.76 | C |
| ATOM | 6045 | CH2 | TRP | B | 616 | 9.671 | 12.560 | 102.869 | 1.00 | 35.47 | C |
| ATOM | 6046 | N | HIS | B | 617 | 11.198 | 18.031 | 98.159 | 1.00 | 35.00 | N |
| ATOM | 6047 | CA | HIS | B | 617 | 12.153 | 18.823 | 97.396 | 1.00 | 34.53 | C |
| ATOM | 6048 | C | HIS | B | 617 | 13.523 | 18.172 | 97.464 | 1.00 | 34.18 | C |
| ATOM | 6049 | O | HIS | B | 617 | 13.822 | 17.431 | 98.403 | 1.00 | 34.32 | O |
| ATOM | 6050 | CB | HIS | B | 617 | 12.221 | 20.253 | 97.927 | 1.00 | 34.45 | C |
| ATOM | 6051 | CG | HIS | B | 617 | 10.891 | 20.932 | 97.996 | 1.00 | 34.78 | C |
| ATOM | 6052 | ND1 | HIS | B | 617 | 10.339 | 21.601 | 96.923 | 1.00 | 34.89 | N |
| ATOM | 6053 | CD2 | HIS | B | 617 | 9.995 | 21.035 | 99.006 | 1.00 | 34.79 | C |
| ATOM | 6054 | CE1 | HIS | B | 617 | 9.164 | 22.093 | 97.273 | 1.00 | 35.00 | C |
| ATOM | 6055 | NE2 | HIS | B | 617 | 8.932 | 21.763 | 98.532 | 1.00 | 35.11 | N |
| ATOM | 6056 | N | ALA | B | 618 | 14.343 | 18.438 | 96.455 | 1.00 | 33.67 | N |
| ATOM | 6057 | CA | ALA | B | 618 | 15.707 | 17.928 | 96.415 | 1.00 | 33.24 | C |
| ATOM | 6058 | C | ALA | B | 618 | 16.671 | 18.941 | 97.008 | 1.00 | 32.94 | C |
| ATOM | 6059 | O | ALA | B | 618 | 16.612 | 20.134 | 96.693 | 1.00 | 32.87 | O |
| ATOM | 6060 | CB | ALA | B | 618 | 16.108 | 17.593 | 94.983 | 1.00 | 33.16 | C |
| ATOM | 6061 | N | GLY | B | 619 | 17.555 | 18.466 | 97.876 | 1.00 | 32.58 | N |
| ATOM | 6062 | CA | GLY | B | 619 | 18.667 | 19.285 | 98.339 | 1.00 | 32.27 | C |
| ATOM | 6063 | C | GLY | B | 619 | 19.746 | 19.300 | 97.275 | 1.00 | 31.96 | C |
| ATOM | 6064 | O | GLY | B | 619 | 19.709 | 18.499 | 96.347 | 1.00 | 31.84 | O |
| ATOM | 6065 | N | GLU | B | 620 | 20.694 | 20.226 | 97.393 | 1.00 | 31.89 | N |
| ATOM | 6066 | CA | GLU | B | 620 | 21.864 | 20.240 | 96.520 | 1.00 | 31.82 | C |
| ATOM | 6067 | C | GLU | B | 620 | 22.785 | 19.098 | 96.913 | 1.00 | 31.67 | C |
| ATOM | 6068 | O | GLU | B | 620 | 22.819 | 18.694 | 98.073 | 1.00 | 31.28 | O |
| ATOM | 6069 | CB | GLU | B | 620 | 22.629 | 21.550 | 96.652 | 1.00 | 31.88 | C |
| ATOM | 6070 | CG | GLU | B | 620 | 21.887 | 22.776 | 96.183 | 1.00 | 32.68 | C |
| ATOM | 6071 | CD | GLU | B | 620 | 22.734 | 24.032 | 96.277 | 1.00 | 33.77 | C |
| ATOM | 6072 | OE1 | GLU | B | 620 | 23.886 | 23.955 | 96.764 | 1.00 | 34.06 | O |
| ATOM | 6073 | OE2 | GLU | B | 620 | 22.246 | 25.104 | 95.861 | 1.00 | 35.05 | O |
| ATOM | 6074 | N | ALA | B | 621 | 23.531 | 18.583 | 95.941 | 1.00 | 31.77 | N |
| ATOM | 6075 | CA | ALA | B | 621 | 24.474 | 17.501 | 96.194 | 1.00 | 31.76 | C |
| ATOM | 6076 | C | ALA | B | 621 | 25.675 | 17.989 | 97.000 | 1.00 | 31.80 | C |
| ATOM | 6077 | O | ALA | B | 621 | 26.023 | 19.170 | 96.966 | 1.00 | 31.73 | O |
| ATOM | 6078 | CB | ALA | B | 621 | 24.924 | 16.870 | 94.884 | 1.00 | 31.51 | C |
| ATOM | 6079 | N | VAL | B | 622 | 26.301 | 17.069 | 97.728 | 1.00 | 32.03 | N |
| ATOM | 6080 | CA | VAL | B | 622 | 27.564 | 17.351 | 98.408 | 1.00 | 32.21 | C |
| ATOM | 6081 | C | VAL | B | 622 | 28.626 | 17.706 | 97.368 | 1.00 | 32.39 | C |
| ATOM | 6082 | O | VAL | B | 622 | 29.471 | 18.580 | 97.603 | 1.00 | 32.51 | O |
| ATOM | 6083 | CB | VAL | B | 622 | 28.029 | 16.148 | 99.266 | 1.00 | 32.18 | C |
| ATOM | 6084 | CG1 | VAL | B | 622 | 29.389 | 16.420 | 99.885 | 1.00 | 31.98 | C |
| ATOM | 6085 | CG2 | VAL | B | 622 | 27.001 | 15.831 | 100.348 | 1.00 | 31.89 | C |
| ATOM | 6086 | N | ASN | B | 623 | 28.554 | 17.034 | 96.217 | 1.00 | 32.49 | N |
| ATOM | 6087 | CA | ASN | B | 623 | 29.464 | 17.268 | 95.093 | 1.00 | 32.84 | C |
| ATOM | 6088 | C | ASN | B | 623 | 29.177 | 18.504 | 94.229 | 1.00 | 33.29 | C |
| ATOM | 6089 | O | ASN | B | 623 | 29.893 | 18.747 | 93.259 | 1.00 | 33.57 | O |
| ATOM | 6090 | CB | ASN | B | 623 | 29.531 | 16.026 | 94.196 | 1.00 | 32.75 | C |
| ATOM | 6091 | CG | ASN | B | 623 | 30.187 | 14.846 | 94.879 | 1.00 | 31.84 | C |
| ATOM | 6092 | OD1 | ASN | B | 623 | 31.038 | 15.010 | 95.747 | 1.00 | 31.67 | O |
| ATOM | 6093 | ND2 | ASN | B | 623 | 29.790 | 13.649 | 94.489 | 1.00 | 30.91 | N |
| ATOM | 6094 | N | ASP | B | 624 | 28.146 | 19.279 | 94.562 | 1.00 | 33.92 | N |
| ATOM | 6095 | CA | ASP | B | 624 | 27.834 | 20.490 | 93.793 | 1.00 | 34.82 | C |
| ATOM | 6096 | C | ASP | B | 624 | 28.737 | 21.660 | 94.169 | 1.00 | 35.79 | C |
| ATOM | 6097 | O | ASP | B | 624 | 28.775 | 22.079 | 95.332 | 1.00 | 35.82 | O |
| ATOM | 6098 | CB | ASP | B | 624 | 26.360 | 20.881 | 93.933 | 1.00 | 34.49 | C |
| ATOM | 6099 | CG | ASP | B | 624 | 25.428 | 19.947 | 93.172 | 1.00 | 34.02 | C |
| ATOM | 6100 | OD1 | ASP | B | 624 | 25.914 | 19.122 | 92.368 | 1.00 | 33.13 | O |
| ATOM | 6101 | OD2 | ASP | B | 624 | 24.200 | 20.038 | 93.387 | 1.00 | 33.13 | O |
| ATOM | 6102 | N | ASN | B | 625 | 29.450 | 22.181 | 93.166 | 1.00 | 36.95 | N |
| ATOM | 6103 | CA | ASN | B | 625 | 30.427 | 23.269 | 93.328 | 1.00 | 38.11 | C |
| ATOM | 6104 | C | ASN | B | 625 | 31.481 | 22.944 | 94.391 | 1.00 | 38.93 | C |
| ATOM | 6105 | O | ASN | B | 625 | 31.882 | 23.799 | 95.187 | 1.00 | 39.20 | O |
| ATOM | 6106 | CB | ASN | B | 625 | 29.734 | 24.617 | 93.593 | 1.00 | 38.18 | C |
| ATOM | 6107 | CG | ASN | B | 625 | 30.598 | 25.813 | 93.195 | 1.00 | 38.89 | C |
| ATOM | 6108 | OD1 | ASN | B | 625 | 31.236 | 25.818 | 92.135 | 1.00 | 39.62 | O |
| ATOM | 6109 | ND2 | ASN | B | 625 | 30.620 | 26.831 | 94.048 | 1.00 | 38.89 | N |
| ATOM | 6110 | N | ARG | B | 626 | 31.920 | 21.688 | 94.384 | 1.00 | 39.83 | N |
| ATOM | 6111 | CA | ARG | B | 626 | 32.857 | 21.182 | 95.368 | 1.00 | 40.63 | C |
| ATOM | 6112 | C | ARG | B | 626 | 34.268 | 21.167 | 94.793 | 1.00 | 41.43 | C |
| ATOM | 6113 | O | ARG | B | 626 | 34.540 | 20.498 | 93.793 | 1.00 | 41.60 | O |
| ATOM | 6114 | CB | ARG | B | 626 | 32.446 | 19.776 | 95.805 | 1.00 | 40.47 | C |
| ATOM | 6115 | CG | ARG | B | 626 | 33.412 | 19.116 | 96.767 | 1.00 | 40.21 | C |
| ATOM | 6116 | CD | ARG | B | 626 | 33.204 | 17.621 | 96.769 | 1.00 | 40.05 | C |
| ATOM | 6117 | NE | ARG | B | 626 | 34.335 | 16.903 | 97.349 | 1.00 | 39.64 | N |
| ATOM | 6118 | CZ | ARG | B | 626 | 34.540 | 15.596 | 97.216 | 1.00 | 39.83 | C |
| ATOM | 6119 | NH1 | ARG | B | 626 | 33.693 | 14.850 | 96.518 | 1.00 | 39.11 | N |
| ATOM | 6120 | NH2 | ARG | B | 626 | 35.597 | 15.030 | 97.787 | 1.00 | 40.07 | N |
| ATOM | 6121 | N | GLN | B | 627 | 35.156 | 21.918 | 95.435 | 1.00 | 42.26 | N |
| ATOM | 6122 | CA | GLN | B | 627 | 36.563 | 21.948 | 95.062 | 1.00 | 42.98 | C |
| ATOM | 6123 | C | GLN | B | 627 | 37.224 | 20.604 | 95.379 | 1.00 | 43.14 | C |
| ATOM | 6124 | O | GLN | B | 627 | 37.285 | 20.187 | 96.537 | 1.00 | 43.14 | O |
| ATOM | 6125 | CB | GLN | B | 627 | 37.269 | 23.096 | 95.790 | 1.00 | 43.22 | C |
| ATOM | 6126 | CG | GLN | B | 627 | 38.794 | 23.020 | 95.799 | 1.00 | 44.30 | C |
| ATOM | 6127 | CD | GLN | B | 627 | 39.440 | 23.667 | 94.590 | 1.00 | 45.55 | C |
| ATOM | 6128 | OE1 | GLN | B | 627 | 38.766 | 24.256 | 93.738 | 1.00 | 46.26 | O |
| ATOM | 6129 | NE2 | GLN | B | 627 | 40.763 | 23.570 | 94.517 | 1.00 | 46.09 | N |
| ATOM | 6130 | N | VAL | B | 628 | 37.686 | 19.923 | 94.333 | 1.00 | 43.46 | N |
| ATOM | 6131 | CA | VAL | B | 628 | 38.444 | 18.680 | 94.475 | 1.00 | 43.59 | C |
| ATOM | 6132 | C | VAL | B | 628 | 39.519 | 18.583 | 93.383 | 1.00 | 43.77 | C |
| ATOM | 6133 | O | VAL | B | 628 | 39.233 | 18.759 | 92.196 | 1.00 | 43.71 | O |
| ATOM | 6134 | CB | VAL | B | 628 | 37.521 | 17.421 | 94.517 | 1.00 | 43.63 | C |
| ATOM | 6135 | CG1 | VAL | B | 628 | 36.739 | 17.247 | 93.215 | 1.00 | 43.52 | C |
| ATOM | 6136 | CG2 | VAL | B | 628 | 38.321 | 16.160 | 94.861 | 1.00 | 43.72 | C |
| ATOM | 6137 | N | ASP | B | 629 | 40.759 | 18.337 | 93.810 | 1.00 | 44.06 | N |
| ATOM | 6138 | CA | ASP | B | 629 | 41.914 | 18.215 | 92.911 | 1.00 | 44.20 | C |
| ATOM | 6139 | C | ASP | B | 629 | 42.149 | 19.495 | 92.099 | 1.00 | 44.03 | C |
| ATOM | 6140 | O | ASP | B | 629 | 42.469 | 19.447 | 90.906 | 1.00 | 43.89 | O |
| ATOM | 6141 | CB | ASP | B | 629 | 41.768 | 16.982 | 91.999 | 1.00 | 44.39 | C |
| ATOM | 6142 | CG | ASP | B | 629 | 41.463 | 15.702 | 92.779 | 1.00 | 45.17 | C |
| ATOM | 6143 | OD1 | ASP | B | 629 | 41.671 | 15.674 | 94.018 | 1.00 | 45.33 | O |
| ATOM | 6144 | OD2 | ASP | B | 629 | 41.013 | 14.721 | 92.147 | 1.00 | 45.84 | O |
| ATOM | 6145 | N | GLY | B | 630 | 41.980 | 20.639 | 92.762 | 1.00 | 43.84 | N |
| ATOM | 6146 | CA | GLY | B | 630 | 42.144 | 21.947 | 92.120 | 1.00 | 43.49 | C |
| ATOM | 6147 | C | GLY | B | 630 | 40.889 | 22.476 | 91.445 | 1.00 | 43.15 | C |
| ATOM | 6148 | O | GLY | B | 630 | 40.653 | 23.689 | 91.419 | 1.00 | 43.21 | O |
| ATOM | 6149 | N | GLN | B | 631 | 40.080 | 21.560 | 90.910 | 1.00 | 42.72 | N |
| ATOM | 6150 | CA | GLN | B | 631 | 38.909 | 21.908 | 90.101 | 1.00 | 42.08 | C |
| ATOM | 6151 | C | GLN | B | 631 | 37.596 | 21.871 | 90.888 | 1.00 | 41.27 | C |
| ATOM | 6152 | O | GLN | B | 631 | 37.486 | 21.179 | 91.902 | 1.00 | 41.26 | O |
| ATOM | 6153 | CB | GLN | B | 631 | 38.813 | 20.980 | 88.880 | 1.00 | 42.45 | C |
| ATOM | 6154 | CG | GLN | B | 631 | 40.034 | 20.985 | 87.945 | 1.00 | 43.30 | C |
| ATOM | 6155 | CD | GLN | B | 631 | 40.276 | 22.334 | 87.281 | 1.00 | 44.74 | C |
| ATOM | 6156 | OE1 | GLN | B | 631 | 40.769 | 23.272 | 87.914 | 1.00 | 45.85 | O |
| ATOM | 6157 | NE2 | GLN | B | 631 | 39.942 | 22.433 | 85.995 | 1.00 | 44.75 | N |
| ATOM | 6158 | N | LYS | B | 632 | 36.610 | 22.628 | 90.413 | 1.00 | 40.17 | N |
| ATOM | 6159 | CA | LYS | B | 632 | 35.259 | 22.591 | 90.962 | 1.00 | 39.08 | C |
| ATOM | 6160 | C | LYS | B | 632 | 34.406 | 21.650 | 90.117 | 1.00 | 38.05 | C |
| ATOM | 6161 | O | LYS | B | 632 | 34.397 | 21.758 | 88.892 | 1.00 | 38.10 | O |
| ATOM | 6162 | CB | LYS | B | 632 | 34.641 | 23.991 | 90.980 | 1.00 | 39.12 | C |
| ATOM | 6163 | CG | LYS | B | 632 | 35.232 | 24.931 | 92.025 | 1.00 | 39.55 | C |
| ATOM | 6164 | CD | LYS | B | 632 | 34.617 | 26.323 | 91.930 | 1.00 | 39.56 | C |
| ATOM | 6165 | CE | LYS | B | 632 | 35.340 | 27.329 | 92.827 | 1.00 | 40.31 | C |
| ATOM | 6166 | NZ | LYS | B | 632 | 35.048 | 27.142 | 94.281 | 1.00 | 39.87 | N |
| ATOM | 6167 | N | ILE | B | 633 | 33.709 | 20.723 | 90.771 | 1.00 | 36.62 | N |
| ATOM | 6168 | CA | ILE | B | 633 | 32.791 | 19.802 | 90.085 | 1.00 | 35.21 | C |
| ATOM | 6169 | C | ILE | B | 633 | 31.311 | 20.083 | 90.407 | 1.00 | 34.11 | C |
| ATOM | 6170 | O | ILE | B | 633 | 30.989 | 20.865 | 91.304 | 1.00 | 33.95 | O |
| ATOM | 6171 | CB | ILE | B | 633 | 33.114 | 18.305 | 90.386 | 1.00 | 35.23 | C |
| ATOM | 6172 | CG1 | ILE | B | 633 | 33.095 | 18.029 | 91.897 | 1.00 | 35.46 | C |
| ATOM | 6173 | CG2 | ILE | B | 633 | 34.442 | 17.899 | 89.753 | 1.00 | 35.13 | C |
| ATOM | 6174 | CD1 | ILE | B | 633 | 33.029 | 16.548 | 92.276 | 1.00 | 35.24 | C |
| ATOM | 6175 | N | HIS | B | 634 | 30.427 | 19.443 | 89.649 | 1.00 | 32.61 | N |
| ATOM | 6176 | CA | HIS | B | 634 | 28.997 | 19.432 | 89.926 | 1.00 | 31.14 | C |
| ATOM | 6177 | C | HIS | B | 634 | 28.545 | 17.987 | 89.768 | 1.00 | 30.28 | C |
| ATOM | 6178 | O | HIS | B | 634 | 29.146 | 17.241 | 88.999 | 1.00 | 30.19 | O |
| ATOM | 6179 | CB | HIS | B | 634 | 28.269 | 20.333 | 88.929 | 1.00 | 31.15 | C |
| ATOM | 6180 | CG | HIS | B | 634 | 26.860 | 20.660 | 89.314 | 1.00 | 30.24 | C |
| ATOM | 6181 | ND1 | HIS | B | 634 | 25.796 | 19.833 | 89.026 | 1.00 | 29.34 | N |
| ATOM | 6182 | CD2 | HIS | B | 634 | 26.338 | 21.735 | 89.951 | 1.00 | 30.00 | C |
| ATOM | 6183 | CE1 | HIS | B | 634 | 24.680 | 20.380 | 89.472 | 1.00 | 29.34 | C |
| ATOM | 6184 | NE2 | HIS | B | 634 | 24.982 | 21.535 | 90.038 | 1.00 | 30.20 | N |
| ATOM | 6185 | N | SER | B | 635 | 27.506 | 17.583 | 90.495 | 1.00 | 29.11 | N |
| ATOM | 6186 | CA | SER | B | 635 | 26.977 | 16.221 | 90.373 | 1.00 | 28.04 | C |
| ATOM | 6187 | C | SER | B | 635 | 26.431 | 15.915 | 88.969 | 1.00 | 27.70 | C |
| ATOM | 6188 | O | SER | B | 635 | 26.425 | 14.756 | 88.541 | 1.00 | 27.05 | O |
| ATOM | 6189 | CB | SER | B | 635 | 25.908 | 15.942 | 91.436 | 1.00 | 27.88 | C |
| ATOM | 6190 | OG | SER | B | 635 | 24.818 | 16.840 | 91.338 | 1.00 | 26.71 | O |
| ATOM | 6191 | N | SER | B | 636 | 25.989 | 16.958 | 88.262 | 1.00 | 27.32 | N |
| ATOM | 6192 | CA | SER | B | 636 | 25.428 | 16.807 | 86.911 | 1.00 | 27.42 | C |
| ATOM | 6193 | C | SER | B | 636 | 26.478 | 16.561 | 85.823 | 1.00 | 27.66 | C |
| ATOM | 6194 | O | SER | B | 636 | 26.159 | 16.009 | 84.772 | 1.00 | 27.51 | O |
| ATOM | 6195 | CB | SER | B | 636 | 24.521 | 17.997 | 86.532 | 1.00 | 27.18 | C |
| ATOM | 6196 | OG | SER | B | 636 | 25.254 | 19.163 | 86.216 | 1.00 | 26.00 | O |
| ATOM | 6197 | N | THR | B | 637 | 27.724 | 16.960 | 86.082 | 1.00 | 27.97 | N |
| ATOM | 6198 | CA | THR | B | 637 | 28.763 | 16.932 | 85.052 | 1.00 | 28.33 | C |
| ATOM | 6199 | C | THR | B | 637 | 30.034 | 16.143 | 85.402 | 1.00 | 28.89 | C |
| ATOM | 6200 | O | THR | B | 637 | 30.873 | 15.914 | 84.538 | 1.00 | 29.27 | O |
| ATOM | 6201 | CB | THR | B | 637 | 29.141 | 18.364 | 84.603 | 1.00 | 28.37 | C |
| ATOM | 6202 | OG1 | THR | B | 637 | 29.527 | 19.145 | 85.737 | 1.00 | 27.92 | O |
| ATOM | 6203 | CG2 | THR | B | 637 | 27.964 | 19.040 | 83.908 | 1.00 | 27.92 | C |
| ATOM | 6204 | N | MET | B | 638 | 30.167 | 15.711 | 86.652 | 1.00 | 29.49 | N |
| ATOM | 6205 | CA | MET | B | 638 | 31.403 | 15.080 | 87.127 | 1.00 | 29.95 | C |
| ATOM | 6206 | C | MET | B | 638 | 31.715 | 13.724 | 86.486 | 1.00 | 30.08 | C |
| ATOM | 6207 | O | MET | B | 638 | 30.809 | 12.946 | 86.167 | 1.00 | 30.00 | O |
| ATOM | 6208 | CB | MET | B | 638 | 31.378 | 14.927 | 88.653 | 1.00 | 29.81 | C |
| ATOM | 6209 | CG | MET | B | 638 | 30.412 | 13.855 | 89.154 | 1.00 | 30.05 | C |
| ATOM | 6210 | SD | MET | B | 638 | 30.300 | 13.721 | 90.945 | 1.00 | 30.64 | S |
| ATOM | 6211 | CE | MET | B | 638 | 31.896 | 13.016 | 91.361 | 1.00 | 29.91 | C |
| ATOM | 6212 | N | ASN | B | 639 | 33.011 | 13.471 | 86.299 | 1.00 | 30.42 | N |
| ATOM | 6213 | CA | ASN | B | 639 | 33.542 | 12.150 | 85.977 | 1.00 | 30.51 | C |
| ATOM | 6214 | C | ASN | B | 639 | 34.784 | 11.921 | 86.841 | 1.00 | 30.90 | C |
| ATOM | 6215 | O | ASN | B | 639 | 35.918 | 12.150 | 86.408 | 1.00 | 30.95 | O |
| ATOM | 6216 | CB | ASN | B | 639 | 33.863 | 12.037 | 84.480 | 1.00 | 30.39 | C |
| ATOM | 6217 | CG | ASN | B | 639 | 34.277 | 10.615 | 84.055 | 1.00 | 30.13 | C |
| ATOM | 6218 | OD1 | ASN | B | 639 | 34.191 | 9.662 | 84.826 | 1.00 | 30.23 | O |
| ATOM | 6219 | ND2 | ASN | B | 639 | 34.713 | 10.481 | 82.813 | 1.00 | 29.51 | N |
| ATOM | 6220 | N | ASN | B | 640 | 34.554 | 11.502 | 88.083 | 1.00 | 31.45 | N |
| ATOM | 6221 | CA | ASN | B | 640 | 35.633 | 11.296 | 89.053 | 1.00 | 31.97 | C |
| ATOM | 6222 | C | ASN | B | 640 | 35.288 | 10.186 | 90.044 | 1.00 | 32.21 | C |
| ATOM | 6223 | O | ASN | B | 640 | 34.527 | 10.400 | 90.990 | 1.00 | 32.49 | O |
| ATOM | 6224 | CB | ASN | B | 640 | 35.971 | 12.609 | 89.782 | 1.00 | 31.92 | C |
| ATOM | 6225 | CG | ASN | B | 640 | 37.235 | 12.510 | 90.643 | 1.00 | 31.97 | C |
| ATOM | 6226 | OD1 | ASN | B | 640 | 37.501 | 11.490 | 91.281 | 1.00 | 31.32 | O |
| ATOM | 6227 | ND2 | ASN | B | 640 | 38.003 | 13.592 | 90.680 | 1.00 | 31.96 | N |
| ATOM | 6228 | N | ARG | B | 641 | 35.863 | 9.008 | 89.812 | 1.00 | 32.49 | N |
| ATOM | 6229 | CA | ARG | B | 641 | 35.540 | 7.791 | 90.557 | 1.00 | 32.86 | C |
| ATOM | 6230 | C | ARG | B | 641 | 35.703 | 7.959 | 92.064 | 1.00 | 32.87 | C |
| ATOM | 6231 | O | ARG | B | 641 | 34.816 | 7.588 | 92.840 | 1.00 | 32.81 | O |
| ATOM | 6232 | CB | ARG | B | 641 | 36.406 | 6.628 | 90.062 | 1.00 | 32.80 | C |
| ATOM | 6233 | CG | ARG | B | 641 | 35.973 | 5.269 | 90.580 | 1.00 | 33.41 | C |
| ATOM | 6234 | CD | ARG | B | 641 | 36.905 | 4.161 | 90.105 | 1.00 | 33.67 | C |
| ATOM | 6235 | NE | ARG | B | 641 | 36.912 | 3.051 | 91.056 | 1.00 | 35.87 | N |
| ATOM | 6236 | CZ | ARG | B | 641 | 37.665 | 3.004 | 92.157 | 1.00 | 36.27 | C |
| ATOM | 6237 | NH1 | ARG | B | 641 | 38.495 | 4.002 | 92.455 | 1.00 | 36.05 | N |
| ATOM | 6238 | NH2 | ARG | B | 641 | 37.585 | 1.955 | 92.964 | 1.00 | 36.68 | N |
| ATOM | 6239 | N | ARG | B | 642 | 36.831 | 8.537 | 92.463 | 1.00 | 32.85 | N |
| ATOM | 6240 | CA | ARG | B | 642 | 37.184 | 8.666 | 93.872 | 1.00 | 33.07 | C |
| ATOM | 6241 | C | ARG | B | 642 | 36.357 | 9.723 | 94.595 | 1.00 | 32.61 | C |
| ATOM | 6242 | O | ARG | B | 642 | 36.105 | 9.604 | 95.798 | 1.00 | 32.71 | O |
| ATOM | 6243 | CB | ARG | B | 642 | 38.681 | 8.953 | 94.025 | 1.00 | 33.04 | C |
| ATOM | 6244 | CG | ARG | B | 642 | 39.574 | 7.756 | 93.697 | 1.00 | 33.88 | C |
| ATOM | 6245 | CD | ARG | B | 642 | 41.050 | 8.017 | 94.027 | 1.00 | 34.29 | C |
| ATOM | 6246 | NE | ARG | B | 642 | 41.268 | 8.245 | 95.460 | 1.00 | 37.31 | N |
| ATOM | 6247 | CZ | ARG | B | 642 | 41.457 | 7.282 | 96.361 | 1.00 | 37.98 | C |
| ATOM | 6248 | NH1 | ARG | B | 642 | 41.463 | 6.005 | 95.990 | 1.00 | 38.69 | N |
| ATOM | 6249 | NH2 | ARG | B | 642 | 41.641 | 7.598 | 97.638 | 1.00 | 37.84 | N |
| ATOM | 6250 | N | ALA | B | 643 | 35.932 | 10.747 | 93.857 | 1.00 | 32.16 | N |
| ATOM | 6251 | CA | ALA | B | 643 | 35.142 | 11.849 | 94.414 | 1.00 | 31.41 | C |
| ATOM | 6252 | C | ALA | B | 643 | 33.667 | 11.488 | 94.622 | 1.00 | 31.11 | C |
| ATOM | 6253 | O | ALA | B | 643 | 32.887 | 12.299 | 95.132 | 1.00 | 30.94 | O |
| ATOM | 6254 | CB | ALA | B | 643 | 35.264 | 13.074 | 93.532 | 1.00 | 31.43 | C |
| ATOM | 6255 | N | GLN | B | 644 | 33.286 | 10.277 | 94.222 | 1.00 | 30.62 | N |
| ATOM | 6256 | CA | GLN | B | 644 | 31.919 | 9.816 | 94.408 | 1.00 | 30.47 | C |
| ATOM | 6257 | C | GLN | B | 644 | 31.524 | 9.710 | 95.876 | 1.00 | 30.49 | C |
| ATOM | 6258 | O | GLN | B | 644 | 32.357 | 9.480 | 96.751 | 1.00 | 30.49 | O |
| ATOM | 6259 | CB | GLN | B | 644 | 31.696 | 8.471 | 93.720 | 1.00 | 30.52 | C |
| ATOM | 6260 | CG | GLN | B | 644 | 31.484 | 8.568 | 92.220 | 1.00 | 29.92 | C |
| ATOM | 6261 | CD | GLN | B | 644 | 31.467 | 7.215 | 91.541 | 1.00 | 29.58 | C |
| ATOM | 6262 | OE1 | GLN | B | 644 | 30.637 | 6.959 | 90.673 | 1.00 | 30.06 | O |
| ATOM | 6263 | NE2 | GLN | B | 644 | 32.389 | 6.341 | 91.931 | 1.00 | 29.22 | N |
| ATOM | 6264 | N | ASN | B | 645 | 30.240 | 9.919 | 96.127 | 1.00 | 30.46 | N |
| ATOM | 6265 | CA | ASN | B | 645 | 29.640 | 9.644 | 97.415 | 1.00 | 30.49 | C |
| ATOM | 6266 | C | ASN | B | 645 | 28.243 | 9.125 | 97.164 | 1.00 | 30.53 | C |
| ATOM | 6267 | O | ASN | B | 645 | 27.442 | 9.769 | 96.500 | 1.00 | 30.67 | O |
| ATOM | 6268 | CB | ASN | B | 645 | 29.648 | 10.877 | 98.326 | 1.00 | 30.58 | C |
| ATOM | 6269 | CG | ASN | B | 645 | 29.560 | 12.182 | 97.561 | 1.00 | 30.20 | C |
| ATOM | 6270 | OD1 | ASN | B | 645 | 28.510 | 12.539 | 97.028 | 1.00 | 30.62 | O |
| ATOM | 6271 | ND2 | ASN | B | 645 | 30.666 | 12.913 | 97.524 | 1.00 | 30.23 | N |
| ATOM | 6272 | N | THR | B | 646 | 27.961 | 7.939 | 97.678 | 1.00 | 30.74 | N |
| ATOM | 6273 | CA | THR | B | 646 | 26.791 | 7.190 | 97.249 | 1.00 | 30.73 | C |
| ATOM | 6274 | C | THR | B | 646 | 25.737 | 7.151 | 98.353 | 1.00 | 31.05 | C |
| ATOM | 6275 | O | THR | B | 646 | 25.209 | 8.194 | 98.739 | 1.00 | 30.70 | O |
| ATOM | 6276 | CB | THR | B | 646 | 27.192 | 5.774 | 96.829 | 1.00 | 30.69 | C |
| ATOM | 6277 | OG1 | THR | B | 646 | 27.561 | 5.028 | 97.992 | 1.00 | 30.51 | O |
| ATOM | 6278 | CG2 | THR | B | 646 | 28.384 | 5.818 | 95.876 | 1.00 | 30.41 | C |
| ATOM | 6279 | N | GLU | B | 647 | 25.435 | 5.950 | 98.848 | 1.00 | 31.56 | N |
| ATOM | 6280 | CA | GLU | B | 647 | 24.525 | 5.768 | 99.975 | 1.00 | 32.18 | C |
| ATOM | 6281 | C | GLU | B | 647 | 25.054 | 6.515 | 101.186 | 1.00 | 32.43 | C |
| ATOM | 6282 | O | GLU | B | 647 | 26.270 | 6.637 | 101.374 | 1.00 | 32.48 | O |
| ATOM | 6283 | CB | GLU | B | 647 | 24.369 | 4.291 | 100.321 | 1.00 | 32.27 | C |
| ATOM | 6284 | CG | GLU | B | 647 | 23.775 | 3.457 | 99.201 | 1.00 | 32.91 | C |
| ATOM | 6285 | CD | GLU | B | 647 | 24.100 | 1.988 | 99.335 | 1.00 | 33.55 | C |
| ATOM | 6286 | OE1 | GLU | B | 647 | 25.078 | 1.656 | 100.039 | 1.00 | 33.54 | O |
| ATOM | 6287 | OE2 | GLU | B | 647 | 23.382 | 1.163 | 98.730 | 1.00 | 34.03 | O |
| ATOM | 6288 | N | SER | B | 648 | 24.136 | 7.024 | 101.996 | 1.00 | 32.58 | N |
| ATOM | 6289 | CA | SER | B | 648 | 24.510 | 7.835 | 103.134 | 1.00 | 32.94 | C |
| ATOM | 6290 | C | SER | B | 648 | 23.516 | 7.691 | 104.273 | 1.00 | 33.01 | C |
| ATOM | 6291 | O | SER | B | 648 | 22.379 | 7.262 | 104.075 | 1.00 | 32.79 | O |
| ATOM | 6292 | CB | SER | B | 648 | 24.652 | 9.311 | 102.724 | 1.00 | 33.13 | C |
| ATOM | 6293 | OG | SER | B | 648 | 23.469 | 9.802 | 102.113 | 1.00 | 33.64 | O |
| ATOM | 6294 | N | THR | B | 649 | 23.971 | 8.033 | 105.471 | 1.00 | 33.15 | N |
| ATOM | 6295 | CA | THR | B | 649 | 23.102 | 8.109 | 106.627 | 1.00 | 33.32 | C |
| ATOM | 6296 | C | THR | B | 649 | 23.231 | 9.493 | 107.251 | 1.00 | 33.56 | C |
| ATOM | 6297 | O | THR | B | 649 | 24.300 | 10.108 | 107.209 | 1.00 | 33.69 | O |
| ATOM | 6298 | CB | THR | B | 649 | 23.379 | 6.985 | 107.650 | 1.00 | 33.37 | C |
| ATOM | 6299 | OG1 | THR | B | 649 | 22.621 | 7.229 | 108.844 | 1.00 | 33.36 | O |
| ATOM | 6300 | CG2 | THR | B | 649 | 24.868 | 6.883 | 107.993 | 1.00 | 33.03 | C |
| ATOM | 6301 | N | VAL | B | 650 | 22.136 | 9.982 | 107.820 | 1.00 | 33.77 | N |
| ATOM | 6302 | CA | VAL | B | 650 | 22.065 | 11.368 | 108.249 | 1.00 | 34.11 | C |
| ATOM | 6303 | C | VAL | B | 650 | 21.682 | 11.501 | 109.727 | 1.00 | 34.60 | C |
| ATOM | 6304 | O | VAL | B | 650 | 20.816 | 10.776 | 110.222 | 1.00 | 34.57 | O |
| ATOM | 6305 | CB | VAL | B | 650 | 21.100 | 12.182 | 107.326 | 1.00 | 34.04 | C |
| ATOM | 6306 | CG1 | VAL | B | 650 | 19.651 | 11.696 | 107.458 | 1.00 | 33.59 | C |
| ATOM | 6307 | CG2 | VAL | B | 650 | 21.206 | 13.673 | 107.599 | 1.00 | 33.79 | C |
| ATOM | 6308 | N | VAL | B | 651 | 22.355 | 12.415 | 110.423 | 1.00 | 35.13 | N |
| ATOM | 6309 | CA | VAL | B | 651 | 22.017 | 12.759 | 111.805 | 1.00 | 35.93 | C |
| ATOM | 6310 | C | VAL | B | 651 | 22.063 | 14.267 | 112.028 | 1.00 | 36.31 | C |
| ATOM | 6311 | O | VAL | B | 651 | 22.900 | 14.967 | 111.453 | 1.00 | 36.43 | O |
| ATOM | 6312 | CB | VAL | B | 651 | 22.952 | 12.074 | 112.845 | 1.00 | 36.03 | C |
| ATOM | 6313 | CG1 | VAL | B | 651 | 22.773 | 10.571 | 112.836 | 1.00 | 36.31 | C |
| ATOM | 6314 | CG2 | VAL | B | 651 | 24.408 | 12.436 | 112.604 | 1.00 | 36.27 | C |
| ATOM | 6315 | N | GLN | B | 652 | 21.160 | 14.769 | 112.861 | 1.00 | 36.82 | N |
| ATOM | 6316 | CA | GLN | B | 652 | 21.233 | 16.161 | 113.264 | 1.00 | 37.41 | C |
| ATOM | 6317 | C | GLN | B | 652 | 21.910 | 16.273 | 114.623 | 1.00 | 37.78 | C |
| ATOM | 6318 | O | GLN | B | 652 | 21.678 | 15.451 | 115.512 | 1.00 | 37.95 | O |
| ATOM | 6319 | CB | GLN | B | 652 | 19.850 | 16.810 | 113.302 | 1.00 | 37.43 | C |
| ATOM | 6320 | CG | GLN | B | 652 | 19.899 | 18.313 | 113.047 | 1.00 | 37.72 | C |
| ATOM | 6321 | CD | GLN | B | 652 | 18.720 | 19.077 | 113.632 | 1.00 | 38.16 | C |
| ATOM | 6322 | OE1 | GLN | B | 652 | 17.766 | 18.491 | 114.150 | 1.00 | 38.47 | O |
| ATOM | 6323 | NE2 | GLN | B | 652 | 18.786 | 20.402 | 113.548 | 1.00 | 37.82 | N |
| ATOM | 6324 | N | LEU | B | 653 | 22.753 | 17.290 | 114.761 | 1.00 | 38.25 | N |
| ATOM | 6325 | CA | LEU | B | 653 | 23.405 | 17.630 | 116.023 | 1.00 | 38.70 | C |
| ATOM | 6326 | C | LEU | B | 653 | 22.532 | 18.598 | 116.816 | 1.00 | 39.15 | C |
| ATOM | 6327 | O | LEU | B | 653 | 21.577 | 19.162 | 116.273 | 1.00 | 39.25 | O |
| ATOM | 6328 | CB | LEU | B | 653 | 24.769 | 18.263 | 115.746 | 1.00 | 38.70 | C |
| ATOM | 6329 | CG | LEU | B | 653 | 25.999 | 17.360 | 115.592 | 1.00 | 38.71 | C |
| ATOM | 6330 | CD1 | LEU | B | 653 | 25.699 | 16.004 | 114.947 | 1.00 | 38.44 | C |
| ATOM | 6331 | CD2 | LEU | B | 653 | 27.103 | 18.091 | 114.842 | 1.00 | 38.52 | C |
| ATOM | 6332 | N | ASN | B | 654 | 22.858 | 18.790 | 118.095 | 1.00 | 39.62 | N |
| ATOM | 6333 | CA | ASN | B | 654 | 22.079 | 19.676 | 118.967 | 1.00 | 40.08 | C |
| ATOM | 6334 | C | ASN | B | 654 | 22.290 | 21.168 | 118.691 | 1.00 | 40.13 | C |
| ATOM | 6335 | O | ASN | B | 654 | 21.469 | 21.997 | 119.087 | 1.00 | 40.19 | O |
| ATOM | 6336 | CB | ASN | B | 654 | 22.346 | 19.368 | 120.443 | 1.00 | 40.32 | C |
| ATOM | 6337 | CG | ASN | B | 654 | 21.733 | 18.053 | 120.892 | 1.00 | 40.87 | C |
| ATOM | 6338 | OD1 | ASN | B | 654 | 20.739 | 17.583 | 120.335 | 1.00 | 41.05 | O |
| ATOM | 6339 | ND2 | ASN | B | 654 | 22.325 | 17.456 | 121.918 | 1.00 | 41.61 | N |
| ATOM | 6340 | N | ASN | B | 655 | 23.388 | 21.504 | 118.017 | 1.00 | 40.19 | N |
| ATOM | 6341 | CA | ASN | B | 655 | 23.628 | 22.881 | 117.588 | 1.00 | 40.29 | C |
| ATOM | 6342 | C | ASN | B | 655 | 22.899 | 23.218 | 116.281 | 1.00 | 40.45 | C |
| ATOM | 6343 | O | ASN | B | 655 | 22.902 | 24.368 | 115.837 | 1.00 | 40.61 | O |
| ATOM | 6344 | CB | ASN | B | 655 | 25.130 | 23.177 | 117.478 | 1.00 | 40.22 | C |
| ATOM | 6345 | CG | ASN | B | 655 | 25.822 | 22.350 | 116.412 | 1.00 | 40.34 | C |
| ATOM | 6346 | OD1 | ASN | B | 655 | 25.177 | 21.668 | 115.611 | 1.00 | 39.94 | O |
| ATOM | 6347 | ND2 | ASN | B | 655 | 27.150 | 22.408 | 116.396 | 1.00 | 40.27 | N |
| ATOM | 6348 | N | GLY | B | 656 | 22.278 | 22.208 | 115.674 | 1.00 | 40.28 | N |
| ATOM | 6349 | CA | GLY | B | 656 | 21.464 | 22.410 | 114.481 | 1.00 | 40.20 | C |
| ATOM | 6350 | C | GLY | B | 656 | 22.104 | 21.954 | 113.182 | 1.00 | 40.06 | C |
| ATOM | 6351 | O | GLY | B | 656 | 21.459 | 21.995 | 112.133 | 1.00 | 40.16 | O |
| ATOM | 6352 | N | ASP | B | 657 | 23.366 | 21.524 | 113.253 | 1.00 | 39.76 | N |
| ATOM | 6353 | CA | ASP | B | 657 | 24.093 | 21.012 | 112.093 | 1.00 | 39.45 | C |
| ATOM | 6354 | C | ASP | B | 657 | 23.578 | 19.650 | 111.651 | 1.00 | 39.31 | C |
| ATOM | 6355 | O | ASP | B | 657 | 23.130 | 18.844 | 112.469 | 1.00 | 39.23 | O |
| ATOM | 6356 | CB | ASP | B | 657 | 25.595 | 20.909 | 112.381 | 1.00 | 39.57 | C |
| ATOM | 6357 | CG | ASP | B | 657 | 26.311 | 22.243 | 112.277 | 1.00 | 39.98 | C |
| ATOM | 6358 | OD1 | ASP | B | 657 | 25.646 | 23.267 | 112.021 | 1.00 | 40.62 | O |
| ATOM | 6359 | OD2 | ASP | B | 657 | 27.547 | 22.268 | 112.455 | 1.00 | 40.78 | O |
| ATOM | 6360 | N | VAL | B | 658 | 23.656 | 19.410 | 110.344 | 1.00 | 38.99 | N |
| ATOM | 6361 | CA | VAL | B | 658 | 23.281 | 18.136 | 109.755 | 1.00 | 38.53 | C |
| ATOM | 6362 | C | VAL | B | 658 | 24.554 | 17.447 | 109.277 | 1.00 | 38.26 | C |
| ATOM | 6363 | O | VAL | B | 658 | 25.348 | 18.037 | 108.537 | 1.00 | 38.28 | O |
| ATOM | 6364 | CB | VAL | B | 658 | 22.286 | 18.325 | 108.577 | 1.00 | 38.55 | C |
| ATOM | 6365 | CG1 | VAL | B | 658 | 22.095 | 17.029 | 107.815 | 1.00 | 38.48 | C |
| ATOM | 6366 | CG2 | VAL | B | 658 | 20.941 | 18.850 | 109.075 | 1.00 | 38.16 | C |
| ATOM | 6367 | N | LYS | B | 659 | 24.746 | 16.206 | 109.714 | 1.00 | 37.76 | N |
| ATOM | 6368 | CA | LYS | B | 659 | 25.906 | 15.418 | 109.315 | 1.00 | 37.33 | C |
| ATOM | 6369 | C | LYS | B | 659 | 25.483 | 14.301 | 108.375 | 1.00 | 37.13 | C |
| ATOM | 6370 | O | LYS | B | 659 | 24.463 | 13.647 | 108.593 | 1.00 | 36.88 | O |
| ATOM | 6371 | CB | LYS | B | 659 | 26.632 | 14.839 | 110.537 | 1.00 | 37.31 | C |
| ATOM | 6372 | CG | LYS | B | 659 | 27.303 | 15.875 | 111.445 | 1.00 | 37.18 | C |
| ATOM | 6373 | CD | LYS | B | 659 | 28.607 | 16.418 | 110.863 | 1.00 | 36.75 | C |
| ATOM | 6374 | CE | LYS | B | 659 | 29.096 | 17.630 | 111.648 | 1.00 | 36.02 | C |
| ATOM | 6375 | NZ | LYS | B | 659 | 30.472 | 18.040 | 111.259 | 1.00 | 35.60 | N |
| ATOM | 6376 | N | LEU | B | 660 | 26.276 | 14.092 | 107.329 | 1.00 | 37.06 | N |
| ATOM | 6377 | CA | LEU | B | 660 | 25.977 | 13.079 | 106.322 | 1.00 | 36.95 | C |
| ATOM | 6378 | C | LEU | B | 660 | 27.154 | 12.126 | 106.145 | 1.00 | 36.91 | C |
| ATOM | 6379 | O | LEU | B | 660 | 28.214 | 12.511 | 105.631 | 1.00 | 37.04 | O |
| ATOM | 6380 | CB | LEU | B | 660 | 25.607 | 13.745 | 104.991 | 1.00 | 36.86 | C |
| ATOM | 6381 | CG | LEU | B | 660 | 24.945 | 12.895 | 103.905 | 1.00 | 36.91 | C |
| ATOM | 6382 | CD1 | LEU | B | 660 | 23.559 | 12.412 | 104.334 | 1.00 | 37.17 | C |
| ATOM | 6383 | CD2 | LEU | B | 660 | 24.861 | 13.682 | 102.601 | 1.00 | 36.71 | C |
| ATOM | 6384 | N | PHE | B | 661 | 26.958 | 10.885 | 106.583 | 1.00 | 36.70 | N |
| ATOM | 6385 | CA | PHE | B | 661 | 27.981 | 9.850 | 106.478 | 1.00 | 36.49 | C |
| ATOM | 6386 | C | PHE | B | 661 | 27.794 | 9.100 | 105.172 | 1.00 | 36.20 | C |
| ATOM | 6387 | O | PHE | B | 661 | 26.821 | 8.355 | 105.015 | 1.00 | 36.27 | O |
| ATOM | 6388 | CB | PHE | B | 661 | 27.903 | 8.883 | 107.665 | 1.00 | 36.74 | C |
| ATOM | 6389 | CG | PHE | B | 661 | 28.026 | 9.553 | 109.001 | 1.00 | 36.81 | C |
| ATOM | 6390 | CD1 | PHE | B | 661 | 26.896 | 9.970 | 109.689 | 1.00 | 37.20 | C |
| ATOM | 6391 | CD2 | PHE | B | 661 | 29.274 | 9.773 | 109.567 | 1.00 | 37.20 | C |
| ATOM | 6392 | CE1 | PHE | B | 661 | 27.008 | 10.598 | 110.922 | 1.00 | 37.74 | C |
| ATOM | 6393 | CE2 | PHE | B | 661 | 29.398 | 10.399 | 110.798 | 1.00 | 37.22 | C |
| ATOM | 6394 | CZ | PHE | B | 661 | 28.262 | 10.811 | 111.480 | 1.00 | 37.41 | C |
| ATOM | 6395 | N | MET | B | 662 | 28.737 | 9.290 | 104.250 | 1.00 | 35.65 | N |
| ATOM | 6396 | CA | MET | B | 662 | 28.597 | 8.803 | 102.878 | 1.00 | 35.13 | C |
| ATOM | 6397 | C | MET | B | 662 | 29.526 | 7.640 | 102.549 | 1.00 | 35.17 | C |
| ATOM | 6398 | O | MET | B | 662 | 30.739 | 7.713 | 102.770 | 1.00 | 35.24 | O |
| ATOM | 6399 | CB | MET | B | 662 | 28.846 | 9.938 | 101.882 | 1.00 | 35.15 | C |
| ATOM | 6400 | CG | MET | B | 662 | 28.151 | 11.245 | 102.221 | 1.00 | 34.88 | C |
| ATOM | 6401 | SD | MET | B | 662 | 28.756 | 12.609 | 101.223 | 1.00 | 34.36 | S |
| ATOM | 6402 | CE | MET | B | 662 | 30.406 | 12.865 | 101.881 | 1.00 | 34.45 | C |
| ATOM | 6403 | N | ARG | B | 663 | 28.943 | 6.577 | 102.003 | 1.00 | 34.95 | N |
| ATOM | 6404 | CA | ARG | B | 663 | 29.704 | 5.456 | 101.472 | 1.00 | 34.97 | C |
| ATOM | 6405 | C | ARG | B | 663 | 30.557 | 5.937 | 100.295 | 1.00 | 35.40 | C |
| ATOM | 6406 | O | ARG | B | 663 | 30.040 | 6.526 | 99.336 | 1.00 | 35.40 | O |
| ATOM | 6407 | CB | ARG | B | 663 | 28.754 | 4.333 | 101.046 | 1.00 | 34.72 | C |
| ATOM | 6408 | CG | ARG | B | 663 | 29.364 | 3.270 | 100.151 | 1.00 | 33.52 | C |
| ATOM | 6409 | CD | ARG | B | 663 | 28.285 | 2.352 | 99.644 | 1.00 | 31.63 | C |
| ATOM | 6410 | NE | ARG | B | 663 | 28.770 | 1.410 | 98.641 | 1.00 | 30.82 | N |
| ATOM | 6411 | CZ | ARG | B | 663 | 28.104 | 0.323 | 98.268 | 1.00 | 29.94 | C |
| ATOM | 6412 | NH1 | ARG | B | 663 | 26.934 | 0.046 | 98.819 | 1.00 | 29.40 | N |
| ATOM | 6413 | NH2 | ARG | B | 663 | 28.603 | −0.487 | 97.345 | 1.00 | 29.21 | N |
| ATOM | 6414 | N | GLY | B | 664 | 31.863 | 5.694 | 100.385 | 1.00 | 35.71 | N |
| ATOM | 6415 | CA | GLY | B | 664 | 32.810 | 6.214 | 99.405 | 1.00 | 36.17 | C |
| ATOM | 6416 | C | GLY | B | 664 | 33.890 | 5.226 | 99.029 | 1.00 | 36.59 | C |
| ATOM | 6417 | O | GLY | B | 664 | 33.860 | 4.062 | 99.439 | 1.00 | 36.76 | O |
| ATOM | 6418 | N | LEU | B | 665 | 34.853 | 5.699 | 98.249 | 1.00 | 36.89 | N |
| ATOM | 6419 | CA | LEU | B | 665 | 35.900 | 4.844 | 97.718 | 1.00 | 37.23 | C |
| ATOM | 6420 | C | LEU | B | 665 | 37.297 | 5.274 | 98.168 | 1.00 | 37.62 | C |
| ATOM | 6421 | O | LEU | B | 665 | 38.290 | 4.966 | 97.506 | 1.00 | 37.57 | O |
| ATOM | 6422 | CB | LEU | B | 665 | 35.801 | 4.779 | 96.190 | 1.00 | 37.18 | C |
| ATOM | 6423 | CG | LEU | B | 665 | 34.682 | 3.890 | 95.638 | 1.00 | 36.93 | C |
| ATOM | 6424 | CD1 | LEU | B | 665 | 34.257 | 4.329 | 94.249 | 1.00 | 36.63 | C |
| ATOM | 6425 | CD2 | LEU | B | 665 | 35.092 | 2.412 | 95.644 | 1.00 | 36.88 | C |
| ATOM | 6426 | N | THR | B | 666 | 37.364 | 5.974 | 99.301 | 1.00 | 38.06 | N |
| ATOM | 6427 | CA | THR | B | 666 | 38.646 | 6.360 | 99.897 | 1.00 | 38.66 | C |
| ATOM | 6428 | C | THR | B | 666 | 39.148 | 5.333 | 100.914 | 1.00 | 38.88 | C |
| ATOM | 6429 | O | THR | B | 666 | 40.321 | 5.349 | 101.287 | 1.00 | 38.87 | O |
| ATOM | 6430 | CB | THR | B | 666 | 38.592 | 7.753 | 100.584 | 1.00 | 38.75 | C |
| ATOM | 6431 | OG1 | THR | B | 666 | 37.646 | 7.730 | 101.662 | 1.00 | 39.11 | O |
| ATOM | 6432 | CG2 | THR | B | 666 | 38.211 | 8.844 | 99.590 | 1.00 | 38.80 | C |
| ATOM | 6433 | N | GLY | B | 667 | 38.260 | 4.442 | 101.351 | 1.00 | 39.30 | N |
| ATOM | 6434 | CA | GLY | B | 667 | 38.576 | 3.491 | 102.416 | 1.00 | 39.73 | C |
| ATOM | 6435 | C | GLY | B | 667 | 38.369 | 4.111 | 103.785 | 1.00 | 40.20 | C |
| ATOM | 6436 | O | GLY | B | 667 | 38.405 | 3.419 | 104.804 | 1.00 | 40.13 | O |
| ATOM | 6437 | N | ASP | B | 668 | 38.158 | 5.428 | 103.787 | 1.00 | 40.78 | N |
| ATOM | 6438 | CA | ASP | B | 668 | 37.900 | 6.227 | 104.982 | 1.00 | 41.31 | C |
| ATOM | 6439 | C | ASP | B | 668 | 36.405 | 6.427 | 105.177 | 1.00 | 41.36 | C |
| ATOM | 6440 | O | ASP | B | 668 | 35.604 | 6.106 | 104.300 | 1.00 | 41.66 | O |
| ATOM | 6441 | CB | ASP | B | 668 | 38.546 | 7.614 | 104.834 | 1.00 | 41.62 | C |
| ATOM | 6442 | CG | ASP | B | 668 | 39.972 | 7.664 | 105.350 | 1.00 | 42.29 | C |
| ATOM | 6443 | OD1 | ASP | B | 668 | 40.392 | 6.725 | 106.056 | 1.00 | 43.63 | O |
| ATOM | 6444 | OD2 | ASP | B | 668 | 40.672 | 8.658 | 105.061 | 1.00 | 42.93 | O |
| ATOM | 6445 | N | LEU | B | 669 | 36.038 | 6.969 | 106.332 | 1.00 | 41.26 | N |
| ATOM | 6446 | CA | LEU | B | 669 | 34.698 | 7.487 | 106.538 | 1.00 | 41.04 | C |
| ATOM | 6447 | C | LEU | B | 669 | 34.659 | 8.909 | 105.994 | 1.00 | 41.03 | C |
| ATOM | 6448 | O | LEU | B | 669 | 35.568 | 9.707 | 106.254 | 1.00 | 41.00 | O |
| ATOM | 6449 | CB | LEU | B | 669 | 34.345 | 7.479 | 108.025 | 1.00 | 40.95 | C |
| ATOM | 6450 | CG | LEU | B | 669 | 33.055 | 8.178 | 108.455 | 1.00 | 40.63 | C |
| ATOM | 6451 | CD1 | LEU | B | 669 | 31.834 | 7.316 | 108.176 | 1.00 | 40.02 | C |
| ATOM | 6452 | CD2 | LEU | B | 669 | 33.136 | 8.530 | 109.921 | 1.00 | 40.31 | C |
| ATOM | 6453 | N | GLN | B | 670 | 33.613 | 9.220 | 105.233 | 1.00 | 40.89 | N |
| ATOM | 6454 | CA | GLN | B | 670 | 33.448 | 10.557 | 104.672 | 1.00 | 40.53 | C |
| ATOM | 6455 | C | GLN | B | 670 | 32.218 | 11.249 | 105.254 | 1.00 | 40.66 | C |
| ATOM | 6456 | O | GLN | B | 670 | 31.122 | 10.676 | 105.289 | 1.00 | 40.71 | O |
| ATOM | 6457 | CB | GLN | B | 670 | 33.380 | 10.494 | 103.148 | 1.00 | 40.55 | C |
| ATOM | 6458 | CG | GLN | B | 670 | 34.729 | 10.279 | 102.483 | 1.00 | 39.86 | C |
| ATOM | 6459 | CD | GLN | B | 670 | 34.626 | 10.223 | 100.975 | 1.00 | 39.49 | C |
| ATOM | 6460 | OE1 | GLN | B | 670 | 34.779 | 11.374 | 100.325 | 1.00 | 39.01 | O |
| ATOM | 6461 | NE2 | GLN | B | 670 | 34.419 | 9.158 | 100.398 | 1.00 | 39.28 | N |
| ATOM | 6462 | N | VAL | B | 671 | 32.412 | 12.480 | 105.721 | 1.00 | 40.61 | N |
| ATOM | 6463 | CA | VAL | B | 671 | 31.358 | 13.223 | 106.408 | 1.00 | 40.53 | C |
| ATOM | 6464 | C | VAL | B | 671 | 31.248 | 14.638 | 105.860 | 1.00 | 40.54 | C |
| ATOM | 6465 | O | VAL | B | 671 | 32.255 | 15.328 | 105.701 | 1.00 | 40.65 | O |
| ATOM | 6466 | CB | VAL | B | 671 | 31.602 | 13.284 | 107.941 | 1.00 | 40.73 | C |
| ATOM | 6467 | CG1 | VAL | B | 671 | 30.418 | 13.933 | 108.659 | 1.00 | 40.16 | C |
| ATOM | 6468 | CG2 | VAL | B | 671 | 31.865 | 11.891 | 108.503 | 1.00 | 40.42 | C |
| ATOM | 6469 | N | ALA | B | 672 | 30.014 | 15.054 | 105.576 | 1.00 | 40.45 | N |
| ATOM | 6470 | CA | ALA | B | 672 | 29.715 | 16.402 | 105.098 | 1.00 | 40.37 | C |
| ATOM | 6471 | C | ALA | B | 672 | 28.747 | 17.121 | 106.045 | 1.00 | 40.34 | C |
| ATOM | 6472 | O | ALA | B | 672 | 27.950 | 16.477 | 106.734 | 1.00 | 40.25 | O |
| ATOM | 6473 | CB | ALA | B | 672 | 29.150 | 16.347 | 103.685 | 1.00 | 40.35 | C |
| ATOM | 6474 | N | THR | B | 673 | 28.819 | 18.453 | 106.064 | 1.00 | 40.29 | N |
| ATOM | 6475 | CA | THR | B | 673 | 28.036 | 19.271 | 106.990 | 1.00 | 40.34 | C |
| ATOM | 6476 | C | THR | B | 673 | 27.122 | 20.264 | 106.264 | 1.00 | 40.43 | C |
| ATOM | 6477 | O | THR | B | 673 | 27.548 | 20.954 | 105.329 | 1.00 | 40.35 | O |
| ATOM | 6478 | CB | THR | B | 673 | 28.955 | 20.048 | 107.968 | 1.00 | 40.25 | C |
| ATOM | 6479 | OG1 | THR | B | 673 | 29.985 | 19.182 | 108.453 | 1.00 | 40.57 | O |
| ATOM | 6480 | CG2 | THR | B | 673 | 28.168 | 20.587 | 109.152 | 1.00 | 40.04 | C |
| ATOM | 6481 | N | SER | B | 674 | 25.867 | 20.320 | 106.707 | 1.00 | 40.49 | N |
| ATOM | 6482 | CA | SER | B | 674 | 24.899 | 21.311 | 106.239 | 1.00 | 40.62 | C |
| ATOM | 6483 | C | SER | B | 674 | 24.460 | 22.223 | 107.378 | 1.00 | 40.70 | C |
| ATOM | 6484 | O | SER | B | 674 | 24.114 | 21.749 | 108.462 | 1.00 | 40.73 | O |
| ATOM | 6485 | CB | SER | B | 674 | 23.670 | 20.629 | 105.643 | 1.00 | 40.64 | C |
| ATOM | 6486 | OG | SER | B | 674 | 22.635 | 21.571 | 105.414 | 1.00 | 40.66 | O |
| ATOM | 6487 | N | LYS | B | 675 | 24.462 | 23.528 | 107.117 | 1.00 | 40.88 | N |
| ATOM | 6488 | CA | LYS | B | 675 | 24.060 | 24.526 | 108.116 | 1.00 | 41.07 | C |
| ATOM | 6489 | C | LYS | B | 675 | 22.715 | 25.192 | 107.821 | 1.00 | 41.02 | C |
| ATOM | 6490 | O | LYS | B | 675 | 22.285 | 26.090 | 108.552 | 1.00 | 41.19 | O |
| ATOM | 6491 | CB | LYS | B | 675 | 25.173 | 25.560 | 108.334 | 1.00 | 40.96 | C |
| ATOM | 6492 | CG | LYS | B | 675 | 25.978 | 25.270 | 109.592 | 1.00 | 41.67 | C |
| ATOM | 6493 | CD | LYS | B | 675 | 27.452 | 25.608 | 109.472 | 1.00 | 42.34 | C |
| ATOM | 6494 | CE | LYS | B | 675 | 28.180 | 25.176 | 110.739 | 1.00 | 42.70 | C |
| ATOM | 6495 | NZ | LYS | B | 675 | 29.664 | 25.223 | 110.620 | 1.00 | 43.32 | N |
| ATOM | 6496 | N | ASP | B | 676 | 22.052 | 24.729 | 106.762 | 1.00 | 40.93 | N |
| ATOM | 6497 | CA | ASP | B | 676 | 20.725 | 25.218 | 106.384 | 1.00 | 40.81 | C |
| ATOM | 6498 | C | ASP | B | 676 | 19.693 | 24.089 | 106.330 | 1.00 | 40.64 | C |
| ATOM | 6499 | O | ASP | B | 676 | 18.710 | 24.164 | 105.588 | 1.00 | 40.61 | O |
| ATOM | 6500 | CB | ASP | B | 676 | 20.782 | 25.977 | 105.050 | 1.00 | 40.92 | C |
| ATOM | 6501 | CG | ASP | B | 676 | 21.579 | 25.238 | 103.978 | 1.00 | 41.34 | C |
| ATOM | 6502 | OD1 | ASP | B | 676 | 21.555 | 23.986 | 103.936 | 1.00 | 41.19 | O |
| ATOM | 6503 | OD2 | ASP | B | 676 | 22.236 | 25.924 | 103.170 | 1.00 | 41.38 | O |
| ATOM | 6504 | N | GLY | B | 677 | 19.934 | 23.046 | 107.120 | 1.00 | 40.51 | N |
| ATOM | 6505 | CA | GLY | B | 677 | 18.985 | 21.950 | 107.290 | 1.00 | 40.38 | C |
| ATOM | 6506 | C | GLY | B | 677 | 18.970 | 20.909 | 106.187 | 1.00 | 40.40 | C |
| ATOM | 6507 | O | GLY | B | 677 | 17.936 | 20.282 | 105.943 | 1.00 | 40.23 | O |
| ATOM | 6508 | N | GLY | B | 678 | 20.108 | 20.729 | 105.518 | 1.00 | 40.47 | N |
| ATOM | 6509 | CA | GLY | B | 678 | 20.259 | 19.682 | 104.501 | 1.00 | 40.44 | C |
| ATOM | 6510 | C | GLY | B | 678 | 20.237 | 20.119 | 103.044 | 1.00 | 40.42 | C |
| ATOM | 6511 | O | GLY | B | 678 | 20.337 | 19.284 | 102.149 | 1.00 | 40.42 | O |
| ATOM | 6512 | N | VAL | B | 679 | 20.110 | 21.419 | 102.794 | 1.00 | 40.44 | N |
| ATOM | 6513 | CA | VAL | B | 679 | 20.050 | 21.927 | 101.422 | 1.00 | 40.53 | C |
| ATOM | 6514 | C | VAL | B | 679 | 21.461 | 22.135 | 100.851 | 1.00 | 40.77 | C |
| ATOM | 6515 | O | VAL | B | 679 | 21.855 | 21.465 | 99.899 | 1.00 | 40.75 | O |
| ATOM | 6516 | CB | VAL | B | 679 | 19.193 | 23.222 | 101.311 | 1.00 | 40.40 | C |
| ATOM | 6517 | CG1 | VAL | B | 679 | 18.906 | 23.553 | 99.857 | 1.00 | 40.37 | C |
| ATOM | 6518 | CG2 | VAL | B | 679 | 17.887 | 23.071 | 102.068 | 1.00 | 40.05 | C |
| ATOM | 6519 | N | THR | B | 680 | 22.210 | 23.058 | 101.447 | 1.00 | 41.17 | N |
| ATOM | 6520 | CA | THR | B | 680 | 23.593 | 23.343 | 101.060 | 1.00 | 41.65 | C |
| ATOM | 6521 | C | THR | B | 680 | 24.555 | 22.554 | 101.942 | 1.00 | 41.93 | C |
| ATOM | 6522 | O | THR | B | 680 | 24.216 | 22.202 | 103.069 | 1.00 | 42.06 | O |
| ATOM | 6523 | CB | THR | B | 680 | 23.878 | 24.864 | 101.155 | 1.00 | 41.61 | C |
| ATOM | 6524 | OG1 | THR | B | 680 | 23.286 | 25.526 | 100.030 | 1.00 | 42.10 | O |
| ATOM | 6525 | CG2 | THR | B | 680 | 25.373 | 25.182 | 101.205 | 1.00 | 41.77 | C |
| ATOM | 6526 | N | TRP | B | 681 | 25.743 | 22.268 | 101.416 | 1.00 | 42.27 | N |
| ATOM | 6527 | CA | TRP | B | 681 | 26.790 | 21.572 | 102.159 | 1.00 | 42.70 | C |
| ATOM | 6528 | C | TRP | B | 681 | 28.060 | 22.410 | 102.169 | 1.00 | 43.18 | C |
| ATOM | 6529 | O | TRP | B | 681 | 28.294 | 23.197 | 101.251 | 1.00 | 43.39 | O |
| ATOM | 6530 | CB | TRP | B | 681 | 27.062 | 20.193 | 101.547 | 1.00 | 42.52 | C |
| ATOM | 6531 | CG | TRP | B | 681 | 25.836 | 19.343 | 101.476 | 1.00 | 42.57 | C |
| ATOM | 6532 | CD1 | TRP | B | 681 | 25.009 | 19.189 | 100.404 | 1.00 | 42.40 | C |
| ATOM | 6533 | CD2 | TRP | B | 681 | 25.277 | 18.550 | 102.533 | 1.00 | 42.69 | C |
| ATOM | 6534 | NE1 | TRP | B | 681 | 23.975 | 18.339 | 100.720 | 1.00 | 42.45 | N |
| ATOM | 6535 | CE2 | TRP | B | 681 | 24.114 | 17.934 | 102.022 | 1.00 | 42.23 | C |
| ATOM | 6536 | CE3 | TRP | B | 681 | 25.651 | 18.292 | 103.859 | 1.00 | 42.48 | C |
| ATOM | 6537 | CZ2 | TRP | B | 681 | 23.320 | 17.079 | 102.788 | 1.00 | 42.01 | C |
| ATOM | 6538 | CZ3 | TRP | B | 681 | 24.859 | 17.442 | 104.622 | 1.00 | 42.47 | C |
| ATOM | 6539 | CH2 | TRP | B | 681 | 23.708 | 16.846 | 104.081 | 1.00 | 42.35 | C |
| ATOM | 6540 | N | GLU | B | 682 | 28.872 | 22.247 | 103.210 | 1.00 | 43.77 | N |
| ATOM | 6541 | CA | GLU | B | 682 | 30.123 | 22.993 | 103.338 | 1.00 | 44.40 | C |
| ATOM | 6542 | C | GLU | B | 682 | 31.169 | 22.536 | 102.329 | 1.00 | 44.64 | C |
| ATOM | 6543 | O | GLU | B | 682 | 31.127 | 21.401 | 101.850 | 1.00 | 44.38 | O |
| ATOM | 6544 | CB | GLU | B | 682 | 30.694 | 22.862 | 104.750 | 1.00 | 44.45 | C |
| ATOM | 6545 | CG | GLU | B | 682 | 30.150 | 23.866 | 105.754 | 1.00 | 44.71 | C |
| ATOM | 6546 | CD | GLU | B | 682 | 30.851 | 23.779 | 107.108 | 1.00 | 44.68 | C |
| ATOM | 6547 | OE1 | GLU | B | 682 | 31.979 | 23.236 | 107.179 | 1.00 | 44.39 | O |
| ATOM | 6548 | OE2 | GLU | B | 682 | 30.270 | 24.257 | 108.106 | 1.00 | 45.64 | O |
| ATOM | 6549 | N | LYS | B | 683 | 32.103 | 23.440 | 102.029 | 1.00 | 45.23 | N |
| ATOM | 6550 | CA | LYS | B | 683 | 33.246 | 23.177 | 101.151 | 1.00 | 45.90 | C |
| ATOM | 6551 | C | LYS | B | 683 | 33.833 | 21.785 | 101.327 | 1.00 | 46.24 | C |
| ATOM | 6552 | O | LYS | B | 683 | 34.064 | 21.072 | 100.350 | 1.00 | 46.31 | O |
| ATOM | 6553 | CB | LYS | B | 683 | 34.370 | 24.182 | 101.433 | 1.00 | 45.93 | C |
| ATOM | 6554 | CG | LYS | B | 683 | 34.285 | 25.494 | 100.692 | 1.00 | 46.09 | C |
| ATOM | 6555 | CD | LYS | B | 683 | 35.606 | 26.237 | 100.819 | 1.00 | 46.33 | C |
| ATOM | 6556 | CE | LYS | B | 683 | 35.524 | 27.630 | 100.231 | 1.00 | 46.73 | C |
| ATOM | 6557 | NZ | LYS | B | 683 | 35.371 | 27.610 | 98.744 | 1.00 | 46.64 | N |
| ATOM | 6558 | N | ASP | B | 684 | 34.064 | 21.414 | 102.583 | 1.00 | 46.70 | N |
| ATOM | 6559 | CA | ASP | B | 684 | 34.958 | 20.316 | 102.915 | 1.00 | 47.23 | C |
| ATOM | 6560 | C | ASP | B | 684 | 34.266 | 19.041 | 103.350 | 1.00 | 47.32 | C |
| ATOM | 6561 | O | ASP | B | 684 | 33.252 | 19.068 | 104.048 | 1.00 | 47.24 | O |
| ATOM | 6562 | CB | ASP | B | 684 | 35.958 | 20.760 | 103.987 | 1.00 | 47.36 | C |
| ATOM | 6563 | CG | ASP | B | 684 | 36.894 | 21.846 | 103.493 | 1.00 | 47.92 | C |
| ATOM | 6564 | OD1 | ASP | B | 684 | 37.594 | 21.614 | 102.483 | 1.00 | 48.11 | O |
| ATOM | 6565 | OD2 | ASP | B | 684 | 36.931 | 22.930 | 104.114 | 1.00 | 49.22 | O |
| ATOM | 6566 | N | ILE | B | 685 | 34.844 | 17.929 | 102.909 | 1.00 | 47.68 | N |
| ATOM | 6567 | CA | ILE | B | 685 | 34.452 | 16.598 | 103.338 | 1.00 | 48.00 | C |
| ATOM | 6568 | C | ILE | B | 685 | 35.488 | 16.120 | 104.349 | 1.00 | 48.37 | C |
| ATOM | 6569 | O | ILE | B | 685 | 36.681 | 16.011 | 104.029 | 1.00 | 48.48 | O |
| ATOM | 6570 | CB | ILE | B | 685 | 34.367 | 15.621 | 102.135 | 1.00 | 47.90 | C |
| ATOM | 6571 | CG1 | ILE | B | 685 | 33.134 | 15.941 | 101.282 | 1.00 | 47.90 | C |
| ATOM | 6572 | CG2 | ILE | B | 685 | 34.353 | 14.155 | 102.602 | 1.00 | 47.95 | C |
| ATOM | 6573 | CD1 | ILE | B | 685 | 32.951 | 15.033 | 100.078 | 1.00 | 48.05 | C |
| ATOM | 6574 | N | LYS | B | 686 | 35.034 | 15.864 | 105.574 | 1.00 | 48.57 | N |
| ATOM | 6575 | CA | LYS | B | 686 | 35.904 | 15.329 | 106.614 | 1.00 | 48.86 | C |
| ATOM | 6576 | C | LYS | B | 686 | 36.127 | 13.844 | 106.384 | 1.00 | 48.77 | C |
| ATOM | 6577 | O | LYS | B | 686 | 35.176 | 13.079 | 106.216 | 1.00 | 48.63 | O |
| ATOM | 6578 | CB | LYS | B | 686 | 35.321 | 15.567 | 108.012 | 1.00 | 48.99 | C |
| ATOM | 6579 | CG | LYS | B | 686 | 35.474 | 16.991 | 108.526 | 1.00 | 49.78 | C |
| ATOM | 6580 | CD | LYS | B | 686 | 34.584 | 17.240 | 109.741 | 1.00 | 50.51 | C |
| ATOM | 6581 | CE | LYS | B | 686 | 34.607 | 18.707 | 110.157 | 1.00 | 50.94 | C |
| ATOM | 6582 | NZ | LYS | B | 686 | 33.600 | 19.007 | 111.219 | 1.00 | 51.37 | N |
| ATOM | 6583 | N | ARG | B | 687 | 37.397 | 13.457 | 106.362 | 1.00 | 48.98 | N |
| ATOM | 6584 | CA | ARG | B | 687 | 37.787 | 12.057 | 106.300 | 1.00 | 49.08 | C |
| ATOM | 6585 | C | ARG | B | 687 | 38.231 | 11.603 | 107.686 | 1.00 | 49.30 | C |
| ATOM | 6586 | O | ARG | B | 687 | 39.055 | 12.267 | 108.323 | 1.00 | 49.42 | O |
| ATOM | 6587 | CB | ARG | B | 687 | 38.915 | 11.862 | 105.289 | 1.00 | 49.01 | C |
| ATOM | 6588 | CG | ARG | B | 687 | 38.480 | 11.999 | 103.836 | 1.00 | 48.44 | C |
| ATOM | 6589 | CD | ARG | B | 687 | 39.643 | 11.767 | 102.891 | 1.00 | 47.59 | C |
| ATOM | 6590 | NE | ARG | B | 687 | 40.095 | 10.378 | 102.916 | 1.00 | 47.31 | N |
| ATOM | 6591 | CZ | ARG | B | 687 | 41.181 | 9.928 | 102.293 | 1.00 | 47.30 | C |
| ATOM | 6592 | NH1 | ARG | B | 687 | 41.941 | 10.758 | 101.588 | 1.00 | 47.38 | N |
| ATOM | 6593 | NH2 | ARG | B | 687 | 41.509 | 8.644 | 102.376 | 1.00 | 46.54 | N |
| ATOM | 6594 | N | TYR | B | 688 | 37.667 | 10.491 | 108.157 | 1.00 | 49.37 | N |
| ATOM | 6595 | CA | TYR | B | 688 | 38.048 | 9.913 | 109.447 | 1.00 | 49.52 | C |
| ATOM | 6596 | C | TYR | B | 688 | 38.725 | 8.552 | 109.275 | 1.00 | 49.68 | C |
| ATOM | 6597 | O | TYR | B | 688 | 38.041 | 7.530 | 109.190 | 1.00 | 49.78 | O |
| ATOM | 6598 | CB | TYR | B | 688 | 36.835 | 9.805 | 110.379 | 1.00 | 49.56 | C |
| ATOM | 6599 | CG | TYR | B | 688 | 36.301 | 11.142 | 110.830 | 1.00 | 49.61 | C |
| ATOM | 6600 | CD1 | TYR | B | 688 | 35.131 | 11.669 | 110.281 | 1.00 | 49.34 | C |
| ATOM | 6601 | CD2 | TYR | B | 688 | 36.973 | 11.892 | 111.797 | 1.00 | 49.74 | C |
| ATOM | 6602 | CE1 | TYR | B | 688 | 34.640 | 12.907 | 110.688 | 1.00 | 49.34 | C |
| ATOM | 6603 | CE2 | TYR | B | 688 | 36.492 | 13.129 | 112.211 | 1.00 | 49.56 | C |
| ATOM | 6604 | CZ | TYR | B | 688 | 35.327 | 13.630 | 111.653 | 1.00 | 49.62 | C |
| ATOM | 6605 | OH | TYR | B | 688 | 34.849 | 14.852 | 112.066 | 1.00 | 49.63 | O |
| ATOM | 6606 | N | PRO | B | 689 | 40.075 | 8.537 | 109.221 | 1.00 | 49.86 | N |
| ATOM | 6607 | CA | PRO | B | 689 | 40.873 | 7.312 | 109.052 | 1.00 | 49.87 | C |
| ATOM | 6608 | C | PRO | B | 689 | 40.729 | 6.298 | 110.189 | 1.00 | 49.96 | C |
| ATOM | 6609 | O | PRO | B | 689 | 40.927 | 5.097 | 109.964 | 1.00 | 49.84 | O |
| ATOM | 6610 | CB | PRO | B | 689 | 42.314 | 7.833 | 108.985 | 1.00 | 49.78 | C |
| ATOM | 6611 | CG | PRO | B | 689 | 42.272 | 9.155 | 109.643 | 1.00 | 49.94 | C |
| ATOM | 6612 | CD | PRO | B | 689 | 40.935 | 9.731 | 109.312 | 1.00 | 49.88 | C |
| ATOM | 6613 | N | GLN | B | 690 | 40.386 | 6.781 | 111.386 | 1.00 | 50.02 | N |
| ATOM | 6614 | CA | GLN | B | 690 | 40.162 | 5.918 | 112.551 | 1.00 | 50.19 | C |
| ATOM | 6615 | C | GLN | B | 690 | 38.971 | 4.979 | 112.341 | 1.00 | 50.18 | C |
| ATOM | 6616 | O | GLN | B | 690 | 38.780 | 4.035 | 113.110 | 1.00 | 50.24 | O |
| ATOM | 6617 | CB | GLN | B | 690 | 39.946 | 6.741 | 113.831 | 1.00 | 50.32 | C |
| ATOM | 6618 | CG | GLN | B | 690 | 41.043 | 7.753 | 114.170 | 1.00 | 50.65 | C |
| ATOM | 6619 | CD | GLN | B | 690 | 40.742 | 9.157 | 113.645 | 1.00 | 51.51 | C |
| ATOM | 6620 | OE1 | GLN | B | 690 | 39.580 | 9.535 | 113.463 | 1.00 | 51.72 | O |
| ATOM | 6621 | NE2 | GLN | B | 690 | 41.792 | 9.937 | 113.407 | 1.00 | 51.29 | N |
| ATOM | 6622 | N | VAL | B | 691 | 38.173 | 5.256 | 111.308 | 1.00 | 50.12 | N |
| ATOM | 6623 | CA | VAL | B | 691 | 37.027 | 4.422 | 110.930 | 1.00 | 49.96 | C |
| ATOM | 6624 | C | VAL | B | 691 | 37.199 | 3.911 | 109.494 | 1.00 | 49.82 | C |
| ATOM | 6625 | O | VAL | B | 691 | 37.492 | 4.688 | 108.578 | 1.00 | 49.87 | O |
| ATOM | 6626 | CB | VAL | B | 691 | 35.682 | 5.197 | 111.075 | 1.00 | 50.20 | C |
| ATOM | 6627 | CG1 | VAL | B | 691 | 34.504 | 4.400 | 110.495 | 1.00 | 49.82 | C |
| ATOM | 6628 | CG2 | VAL | B | 691 | 35.421 | 5.566 | 112.543 | 1.00 | 49.91 | C |
| ATOM | 6629 | N | LYS | B | 692 | 37.024 | 2.604 | 109.308 | 1.00 | 49.51 | N |
| ATOM | 6630 | CA | LYS | B | 692 | 37.158 | 1.988 | 107.988 | 1.00 | 49.15 | C |
| ATOM | 6631 | C | LYS | B | 692 | 35.842 | 1.973 | 107.217 | 1.00 | 48.80 | C |
| ATOM | 6632 | O | LYS | B | 692 | 34.763 | 1.921 | 107.812 | 1.00 | 48.51 | O |
| ATOM | 6633 | CB | LYS | B | 692 | 37.719 | 0.562 | 108.099 | 1.00 | 49.19 | C |
| ATOM | 6634 | CG | LYS | B | 692 | 39.212 | 0.485 | 108.425 | 1.00 | 49.43 | C |
| ATOM | 6635 | CD | LYS | B | 692 | 40.085 | 0.769 | 107.203 | 1.00 | 49.70 | C |
| ATOM | 6636 | CE | LYS | B | 692 | 41.567 | 0.776 | 107.557 | 1.00 | 50.10 | C |
| ATOM | 6637 | NZ | LYS | B | 692 | 41.951 | 1.968 | 108.372 | 1.00 | 50.65 | N |
| ATOM | 6638 | N | ASP | B | 693 | 35.958 | 2.025 | 105.890 | 1.00 | 48.52 | N |
| ATOM | 6639 | CA | ASP | B | 693 | 34.830 | 1.869 | 104.968 | 1.00 | 48.19 | C |
| ATOM | 6640 | C | ASP | B | 693 | 35.309 | 1.042 | 103.782 | 1.00 | 47.97 | C |
| ATOM | 6641 | O | ASP | B | 693 | 36.155 | 1.486 | 103.001 | 1.00 | 48.09 | O |
| ATOM | 6642 | CB | ASP | B | 693 | 34.306 | 3.243 | 104.509 | 1.00 | 48.18 | C |
| ATOM | 6643 | CG | ASP | B | 693 | 33.185 | 3.151 | 103.461 | 1.00 | 48.23 | C |
| ATOM | 6644 | OD1 | ASP | B | 693 | 32.438 | 2.146 | 103.428 | 1.00 | 48.53 | O |
| ATOM | 6645 | OD2 | ASP | B | 693 | 33.044 | 4.110 | 102.670 | 1.00 | 47.72 | O |
| ATOM | 6646 | N | VAL | B | 694 | 34.772 | −0.166 | 103.658 | 1.00 | 47.60 | N |
| ATOM | 6647 | CA | VAL | B | 694 | 35.160 | −1.076 | 102.577 | 1.00 | 47.20 | C |
| ATOM | 6648 | C | VAL | B | 694 | 34.241 | −0.942 | 101.359 | 1.00 | 47.03 | C |
| ATOM | 6649 | O | VAL | B | 694 | 34.066 | −1.896 | 100.593 | 1.00 | 46.86 | O |
| ATOM | 6650 | CB | VAL | B | 694 | 35.221 | −2.547 | 103.061 | 1.00 | 47.17 | C |
| ATOM | 6651 | CG1 | VAL | B | 694 | 36.454 | −2.772 | 103.926 | 1.00 | 47.07 | C |
| ATOM | 6652 | CG2 | VAL | B | 694 | 33.949 | −2.928 | 103.810 | 1.00 | 46.92 | C |
| ATOM | 6653 | N | TYR | B | 695 | 33.680 | 0.258 | 101.190 | 1.00 | 46.94 | N |
| ATOM | 6654 | CA | TYR | B | 695 | 32.687 | 0.574 | 100.150 | 1.00 | 46.79 | C |
| ATOM | 6655 | C | TYR | B | 695 | 31.403 | −0.251 | 100.317 | 1.00 | 46.59 | C |
| ATOM | 6656 | O | TYR | B | 695 | 31.110 | −1.165 | 99.539 | 1.00 | 46.43 | O |
| ATOM | 6657 | CB | TYR | B | 695 | 33.288 | 0.468 | 98.736 | 1.00 | 46.61 | C |
| ATOM | 6658 | CG | TYR | B | 695 | 32.411 | 1.024 | 97.632 | 1.00 | 46.85 | C |
| ATOM | 6659 | CD1 | TYR | B | 695 | 32.036 | 2.372 | 97.614 | 1.00 | 46.66 | C |
| ATOM | 6660 | CD2 | TYR | B | 695 | 31.969 | 0.205 | 96.591 | 1.00 | 46.80 | C |
| ATOM | 6661 | CE1 | TYR | B | 695 | 31.239 | 2.883 | 96.594 | 1.00 | 46.55 | C |
| ATOM | 6662 | CE2 | TYR | B | 695 | 31.174 | 0.708 | 95.565 | 1.00 | 46.44 | C |
| ATOM | 6663 | CZ | TYR | B | 695 | 30.811 | 2.043 | 95.573 | 1.00 | 46.56 | C |
| ATOM | 6664 | OH | TYR | B | 695 | 30.021 | 2.534 | 94.556 | 1.00 | 46.37 | O |
| ATOM | 6665 | N | VAL | B | 696 | 30.654 | 0.094 | 101.359 | 1.00 | 46.56 | N |
| ATOM | 6666 | CA | VAL | B | 696 | 29.424 | −0.600 | 101.739 | 1.00 | 46.60 | C |
| ATOM | 6667 | C | VAL | B | 696 | 28.543 | 0.382 | 102.527 | 1.00 | 46.71 | C |
| ATOM | 6668 | O | VAL | B | 696 | 29.032 | 1.412 | 103.002 | 1.00 | 46.77 | O |
| ATOM | 6669 | CB | VAL | B | 696 | 29.740 | −1.882 | 102.567 | 1.00 | 46.48 | C |
| ATOM | 6670 | CG1 | VAL | B | 696 | 30.425 | −1.532 | 103.894 | 1.00 | 46.68 | C |
| ATOM | 6671 | CG2 | VAL | B | 696 | 28.487 | −2.728 | 102.791 | 1.00 | 46.48 | C |
| ATOM | 6672 | N | GLN | B | 697 | 27.253 | 0.079 | 102.648 | 1.00 | 46.86 | N |
| ATOM | 6673 | CA | GLN | B | 697 | 26.334 | 0.933 | 103.402 | 1.00 | 47.10 | C |
| ATOM | 6674 | C | GLN | B | 697 | 26.653 | 0.947 | 104.902 | 1.00 | 47.33 | C |
| ATOM | 6675 | O | GLN | B | 697 | 27.111 | −0.050 | 105.467 | 1.00 | 47.16 | O |
| ATOM | 6676 | CB | GLN | B | 697 | 24.883 | 0.509 | 103.149 | 1.00 | 47.03 | C |
| ATOM | 6677 | CG | GLN | B | 697 | 23.803 | 1.352 | 103.830 | 1.00 | 46.80 | C |
| ATOM | 6678 | CD | GLN | B | 697 | 23.380 | 0.792 | 105.181 | 1.00 | 46.92 | C |
| ATOM | 6679 | OE1 | GLN | B | 697 | 23.952 | −0.184 | 105.671 | 1.00 | 47.39 | O |
| ATOM | 6680 | NE2 | GLN | B | 697 | 22.376 | 1.410 | 105.789 | 1.00 | 46.50 | N |
| ATOM | 6681 | N | MET | B | 698 | 26.400 | 2.094 | 105.525 | 1.00 | 47.73 | N |
| ATOM | 6682 | CA | MET | B | 698 | 26.601 | 2.290 | 106.957 | 1.00 | 48.22 | C |
| ATOM | 6683 | C | MET | B | 698 | 25.335 | 2.856 | 107.610 | 1.00 | 48.47 | C |
| ATOM | 6684 | O | MET | B | 698 | 24.393 | 3.229 | 106.914 | 1.00 | 48.55 | O |
| ATOM | 6685 | CB | MET | B | 698 | 27.797 | 3.215 | 107.196 | 1.00 | 48.21 | C |
| ATOM | 6686 | CG | MET | B | 698 | 27.680 | 4.581 | 106.536 | 1.00 | 48.18 | C |
| ATOM | 6687 | SD | MET | B | 698 | 29.260 | 5.427 | 106.401 | 1.00 | 48.35 | S |
| ATOM | 6688 | CE | MET | B | 698 | 30.082 | 4.435 | 105.156 | 1.00 | 48.55 | C |
| ATOM | 6689 | N | SER | B | 699 | 25.320 | 2.916 | 108.941 | 1.00 | 48.82 | N |
| ATOM | 6690 | CA | SER | B | 699 | 24.168 | 3.426 | 109.686 | 1.00 | 49.15 | C |
| ATOM | 6691 | C | SER | B | 699 | 24.617 | 4.254 | 110.883 | 1.00 | 49.42 | C |
| ATOM | 6692 | O | SER | B | 699 | 25.491 | 3.832 | 111.633 | 1.00 | 49.46 | O |
| ATOM | 6693 | CB | SER | B | 699 | 23.287 | 2.265 | 110.145 | 1.00 | 49.06 | C |
| ATOM | 6694 | OG | SER | B | 699 | 22.249 | 2.701 | 110.999 | 1.00 | 49.17 | O |
| ATOM | 6695 | N | ALA | B | 700 | 24.015 | 5.428 | 111.058 | 1.00 | 49.81 | N |
| ATOM | 6696 | CA | ALA | B | 700 | 24.365 | 6.318 | 112.167 | 1.00 | 50.38 | C |
| ATOM | 6697 | C | ALA | B | 700 | 23.153 | 6.959 | 112.839 | 1.00 | 50.88 | C |
| ATOM | 6698 | O | ALA | B | 700 | 22.199 | 7.353 | 112.169 | 1.00 | 50.91 | O |
| ATOM | 6699 | CB | ALA | B | 700 | 25.327 | 7.389 | 111.705 | 1.00 | 50.29 | C |
| ATOM | 6700 | N | ILE | B | 701 | 23.208 | 7.069 | 114.166 | 1.00 | 51.54 | N |
| ATOM | 6701 | CA | ILE | B | 701 | 22.137 | 7.698 | 114.943 | 1.00 | 52.16 | C |
| ATOM | 6702 | C | ILE | B | 701 | 22.662 | 8.664 | 116.003 | 1.00 | 52.73 | C |
| ATOM | 6703 | O | ILE | B | 701 | 23.728 | 8.444 | 116.589 | 1.00 | 52.70 | O |
| ATOM | 6704 | CB | ILE | B | 701 | 21.206 | 6.661 | 115.624 | 1.00 | 52.19 | C |
| ATOM | 6705 | CG1 | ILE | B | 701 | 22.023 | 5.538 | 116.278 | 1.00 | 52.01 | C |
| ATOM | 6706 | CG2 | ILE | B | 701 | 20.175 | 6.122 | 114.624 | 1.00 | 52.08 | C |
| ATOM | 6707 | CD1 | ILE | B | 701 | 21.279 | 4.785 | 117.361 | 1.00 | 52.16 | C |
| ATOM | 6708 | N | HIS | B | 702 | 21.904 | 9.736 | 116.231 | 1.00 | 53.42 | N |
| ATOM | 6709 | CA | HIS | B | 702 | 22.162 | 10.658 | 117.334 | 1.00 | 54.11 | C |
| ATOM | 6710 | C | HIS | B | 702 | 21.631 | 10.068 | 118.641 | 1.00 | 54.67 | C |
| ATOM | 6711 | O | HIS | B | 702 | 20.560 | 9.451 | 118.664 | 1.00 | 54.71 | O |
| ATOM | 6712 | CB | HIS | B | 702 | 21.509 | 12.017 | 117.071 | 1.00 | 54.06 | C |
| ATOM | 6713 | CG | HIS | B | 702 | 21.893 | 13.069 | 118.064 | 1.00 | 54.05 | C |
| ATOM | 6714 | ND1 | HIS | B | 702 | 21.279 | 13.194 | 119.292 | 1.00 | 53.95 | N |
| ATOM | 6715 | CD2 | HIS | B | 702 | 22.840 | 14.036 | 118.017 | 1.00 | 54.05 | C |
| ATOM | 6716 | CE1 | HIS | B | 702 | 21.829 | 14.194 | 119.957 | 1.00 | 54.15 | C |
| ATOM | 6717 | NE2 | HIS | B | 702 | 22.775 | 14.725 | 119.203 | 1.00 | 54.50 | N |
| ATOM | 6718 | N | THR | B | 703 | 22.383 | 10.268 | 119.724 | 1.00 | 55.41 | N |
| ATOM | 6719 | CA | THR | B | 703 | 22.007 | 9.746 | 121.042 | 1.00 | 56.13 | C |
| ATOM | 6720 | C | THR | B | 703 | 22.635 | 10.517 | 122.213 | 1.00 | 56.76 | C |
| ATOM | 6721 | O | THR | B | 703 | 23.855 | 10.642 | 122.311 | 1.00 | 56.73 | O |
| ATOM | 6722 | CB | THR | B | 703 | 22.319 | 8.221 | 121.171 | 1.00 | 56.04 | C |
| ATOM | 6723 | OG1 | THR | B | 703 | 21.857 | 7.737 | 122.437 | 1.00 | 55.79 | O |
| ATOM | 6724 | CG2 | THR | B | 703 | 23.818 | 7.933 | 121.025 | 1.00 | 55.96 | C |
| ATOM | 6725 | N | MET | B | 704 | 21.787 | 11.035 | 123.096 | 1.00 | 57.51 | N |
| ATOM | 6726 | CA | MET | B | 704 | 22.254 | 11.661 | 124.326 | 1.00 | 58.37 | C |
| ATOM | 6727 | C | MET | B | 704 | 22.429 | 10.590 | 125.394 | 1.00 | 58.99 | C |
| ATOM | 6728 | O | MET | B | 704 | 21.593 | 9.685 | 125.516 | 1.00 | 59.21 | O |
| ATOM | 6729 | CB | MET | B | 704 | 21.250 | 12.706 | 124.818 | 1.00 | 58.36 | C |
| ATOM | 6730 | CG | MET | B | 704 | 20.936 | 13.808 | 123.825 | 1.00 | 58.63 | C |
| ATOM | 6731 | SD | MET | B | 704 | 22.359 | 14.854 | 123.507 | 1.00 | 59.47 | S |
| ATOM | 6732 | CE | MET | B | 704 | 22.411 | 15.839 | 125.008 | 1.00 | 59.89 | C |
| ATOM | 6733 | N | HIS | B | 705 | 23.516 | 10.685 | 126.156 | 1.00 | 59.62 | N |
| ATOM | 6734 | CA | HIS | B | 705 | 23.681 | 9.861 | 127.355 | 1.00 | 60.26 | C |
| ATOM | 6735 | C | HIS | B | 705 | 24.406 | 10.597 | 128.479 | 1.00 | 60.65 | C |
| ATOM | 6736 | O | HIS | B | 705 | 25.639 | 10.690 | 128.485 | 1.00 | 60.55 | O |
| ATOM | 6737 | CB | HIS | B | 705 | 24.378 | 8.534 | 127.051 | 1.00 | 60.24 | C |
| ATOM | 6738 | CG | HIS | B | 705 | 24.394 | 7.594 | 128.215 | 1.00 | 60.57 | C |
| ATOM | 6739 | ND1 | HIS | B | 705 | 25.383 | 7.617 | 129.175 | 1.00 | 60.95 | N |
| ATOM | 6740 | CD2 | HIS | B | 705 | 23.529 | 6.622 | 128.587 | 1.00 | 60.91 | C |
| ATOM | 6741 | CE1 | HIS | B | 705 | 25.133 | 6.690 | 130.083 | 1.00 | 61.08 | C |
| ATOM | 6742 | NE2 | HIS | B | 705 | 24.013 | 6.073 | 129.750 | 1.00 | 61.23 | N |
| ATOM | 6743 | N | GLU | B | 706 | 23.614 | 11.120 | 129.417 | 1.00 | 61.19 | N |
| ATOM | 6744 | CA | GLU | B | 706 | 24.100 | 11.853 | 130.597 | 1.00 | 61.71 | C |
| ATOM | 6745 | C | GLU | B | 706 | 24.848 | 13.154 | 130.291 | 1.00 | 61.98 | C |
| ATOM | 6746 | O | GLU | B | 706 | 24.784 | 14.099 | 131.079 | 1.00 | 62.09 | O |
| ATOM | 6747 | CB | GLU | B | 706 | 24.942 | 10.946 | 131.507 | 1.00 | 61.68 | C |
| ATOM | 6748 | CG | GLU | B | 706 | 24.125 | 9.945 | 132.321 | 1.00 | 61.83 | C |
| ATOM | 6749 | CD | GLU | B | 706 | 24.974 | 8.842 | 132.935 | 1.00 | 61.83 | C |
| ATOM | 6750 | OE1 | GLU | B | 706 | 26.216 | 8.990 | 132.982 | 1.00 | 61.80 | O |
| ATOM | 6751 | OE2 | GLU | B | 706 | 24.397 | 7.821 | 133.370 | 1.00 | 61.73 | O |
| ATOM | 6752 | N | GLY | B | 707 | 25.549 | 13.202 | 129.158 | 1.00 | 62.30 | N |
| ATOM | 6753 | CA | GLY | B | 707 | 26.339 | 14.372 | 128.774 | 1.00 | 62.54 | C |
| ATOM | 6754 | C | GLY | B | 707 | 26.142 | 14.778 | 127.328 | 1.00 | 62.70 | C |
| ATOM | 6755 | O | GLY | B | 707 | 25.012 | 14.825 | 126.838 | 1.00 | 62.82 | O |
| ATOM | 6756 | N | LYS | B | 708 | 27.250 | 15.068 | 126.649 | 1.00 | 62.79 | N |
| ATOM | 6757 | CA | LYS | B | 708 | 27.236 | 15.510 | 125.249 | 1.00 | 62.81 | C |
| ATOM | 6758 | C | LYS | B | 708 | 28.391 | 14.902 | 124.437 | 1.00 | 62.61 | C |
| ATOM | 6759 | O | LYS | B | 708 | 29.539 | 14.899 | 124.896 | 1.00 | 62.60 | O |
| ATOM | 6760 | CB | LYS | B | 708 | 27.285 | 17.044 | 125.160 | 1.00 | 62.85 | C |
| ATOM | 6761 | CG | LYS | B | 708 | 25.924 | 17.724 | 125.198 | 1.00 | 63.02 | C |
| ATOM | 6762 | CD | LYS | B | 708 | 26.020 | 19.185 | 124.770 | 1.00 | 63.09 | C |
| ATOM | 6763 | CE | LYS | B | 708 | 24.637 | 19.814 | 124.639 | 1.00 | 63.33 | C |
| ATOM | 6764 | NZ | LYS | B | 708 | 24.703 | 21.300 | 124.602 | 1.00 | 63.21 | N |
| ATOM | 6765 | N | GLU | B | 709 | 28.104 | 14.399 | 123.231 | 1.00 | 62.27 | N |
| ATOM | 6766 | CA | GLU | B | 709 | 26.768 | 14.425 | 122.622 | 1.00 | 61.76 | C |
| ATOM | 6767 | C | GLU | B | 709 | 26.448 | 13.080 | 121.965 | 1.00 | 61.33 | C |
| ATOM | 6768 | O | GLU | B | 709 | 25.286 | 12.692 | 121.880 | 1.00 | 61.32 | O |
| ATOM | 6769 | CB | GLU | B | 709 | 26.670 | 15.577 | 121.613 | 1.00 | 61.85 | C |
| ATOM | 6770 | CG | GLU | B | 709 | 25.275 | 15.878 | 121.068 | 1.00 | 61.87 | C |
| ATOM | 6771 | CD | GLU | B | 709 | 25.243 | 17.133 | 120.202 | 1.00 | 61.81 | C |
| ATOM | 6772 | OE1 | GLU | B | 709 | 25.532 | 18.232 | 120.724 | 1.00 | 62.07 | O |
| ATOM | 6773 | OE2 | GLU | B | 709 | 24.925 | 17.022 | 119.000 | 1.00 | 61.61 | O |
| ATOM | 6774 | N | TYR | B | 710 | 27.492 | 12.399 | 121.490 | 1.00 | 60.80 | N |
| ATOM | 6775 | CA | TYR | B | 710 | 27.452 | 10.983 | 121.057 | 1.00 | 60.34 | C |
| ATOM | 6776 | C | TYR | B | 710 | 26.693 | 10.627 | 119.764 | 1.00 | 59.96 | C |
| ATOM | 6777 | O | TYR | B | 710 | 25.506 | 10.937 | 119.603 | 1.00 | 59.88 | O |
| ATOM | 6778 | CB | TYR | B | 710 | 27.041 | 10.052 | 122.214 | 1.00 | 60.39 | C |
| ATOM | 6779 | CG | TYR | B | 710 | 27.845 | 10.271 | 123.478 | 1.00 | 60.52 | C |
| ATOM | 6780 | CD1 | TYR | B | 710 | 29.191 | 9.910 | 123.539 | 1.00 | 60.55 | C |
| ATOM | 6781 | CD2 | TYR | B | 710 | 27.264 | 10.848 | 124.610 | 1.00 | 60.28 | C |
| ATOM | 6782 | CE1 | TYR | B | 710 | 29.939 | 10.117 | 124.691 | 1.00 | 60.71 | C |
| ATOM | 6783 | CE2 | TYR | B | 710 | 28.004 | 11.058 | 125.770 | 1.00 | 60.42 | C |
| ATOM | 6784 | CZ | TYR | B | 710 | 29.341 | 10.690 | 125.802 | 1.00 | 60.69 | C |
| ATOM | 6785 | OH | TYR | B | 710 | 30.090 | 10.887 | 126.939 | 1.00 | 60.73 | O |
| ATOM | 6786 | N | ILE | B | 711 | 27.414 | 9.965 | 118.857 | 1.00 | 59.38 | N |
| ATOM | 6787 | CA | ILE | B | 711 | 26.844 | 9.346 | 117.657 | 1.00 | 58.85 | C |
| ATOM | 6788 | C | ILE | B | 711 | 27.340 | 7.905 | 117.548 | 1.00 | 58.59 | C |
| ATOM | 6789 | O | ILE | B | 711 | 28.527 | 7.632 | 117.742 | 1.00 | 58.36 | O |
| ATOM | 6790 | CB | ILE | B | 711 | 27.211 | 10.121 | 116.356 | 1.00 | 58.88 | C |
| ATOM | 6791 | CG1 | ILE | B | 711 | 26.386 | 11.408 | 116.239 | 1.00 | 58.70 | C |
| ATOM | 6792 | CG2 | ILE | B | 711 | 27.004 | 9.241 | 115.114 | 1.00 | 58.41 | C |
| ATOM | 6793 | CD1 | ILE | B | 711 | 26.898 | 12.380 | 115.190 | 1.00 | 58.71 | C |
| ATOM | 6794 | N | ILE | B | 712 | 26.421 | 6.993 | 117.244 | 1.00 | 58.32 | N |
| ATOM | 6795 | CA | ILE | B | 712 | 26.765 | 5.600 | 116.983 | 1.00 | 58.12 | C |
| ATOM | 6796 | C | ILE | B | 712 | 26.770 | 5.344 | 115.476 | 1.00 | 57.91 | C |
| ATOM | 6797 | O | ILE | B | 712 | 25.771 | 5.576 | 114.798 | 1.00 | 57.71 | O |
| ATOM | 6798 | CB | ILE | B | 712 | 25.779 | 4.624 | 117.677 | 1.00 | 58.19 | C |
| ATOM | 6799 | CG1 | ILE | B | 712 | 25.626 | 4.970 | 119.165 | 1.00 | 58.34 | C |
| ATOM | 6800 | CG2 | ILE | B | 712 | 26.238 | 3.176 | 117.495 | 1.00 | 57.99 | C |
| ATOM | 6801 | CD1 | ILE | B | 712 | 24.405 | 4.354 | 119.835 | 1.00 | 58.23 | C |
| ATOM | 6802 | N | LEU | B | 713 | 27.905 | 4.872 | 114.966 | 1.00 | 57.78 | N |
| ATOM | 6803 | CA | LEU | B | 713 | 28.052 | 4.523 | 113.554 | 1.00 | 57.79 | C |
| ATOM | 6804 | C | LEU | B | 713 | 28.383 | 3.039 | 113.428 | 1.00 | 57.75 | C |
| ATOM | 6805 | O | LEU | B | 713 | 29.301 | 2.548 | 114.085 | 1.00 | 57.73 | O |
| ATOM | 6806 | CB | LEU | B | 713 | 29.141 | 5.391 | 112.896 | 1.00 | 57.79 | C |
| ATOM | 6807 | CG | LEU | B | 713 | 29.387 | 5.480 | 111.375 | 1.00 | 57.69 | C |
| ATOM | 6808 | CD1 | LEU | B | 713 | 30.290 | 4.371 | 110.844 | 1.00 | 57.58 | C |
| ATOM | 6809 | CD2 | LEU | B | 713 | 28.097 | 5.543 | 110.568 | 1.00 | 57.11 | C |
| ATOM | 6810 | N | SER | B | 714 | 27.634 | 2.330 | 112.588 | 1.00 | 57.80 | N |
| ATOM | 6811 | CA | SER | B | 714 | 27.839 | 0.895 | 112.409 | 1.00 | 57.91 | C |
| ATOM | 6812 | C | SER | B | 714 | 27.999 | 0.491 | 110.945 | 1.00 | 58.00 | C |
| ATOM | 6813 | O | SER | B | 714 | 27.213 | 0.898 | 110.087 | 1.00 | 58.03 | O |
| ATOM | 6814 | CB | SER | B | 714 | 26.699 | 0.108 | 113.058 | 1.00 | 57.92 | C |
| ATOM | 6815 | OG | SER | B | 714 | 26.924 | −1.287 | 112.953 | 1.00 | 57.82 | O |
| ATOM | 6816 | N | ASN | B | 715 | 29.024 | −0.317 | 110.681 | 1.00 | 58.16 | N |
| ATOM | 6817 | CA | ASN | B | 715 | 29.313 | −0.846 | 109.341 | 1.00 | 58.35 | C |
| ATOM | 6818 | C | ASN | B | 715 | 30.270 | −2.043 | 109.395 | 1.00 | 58.46 | C |
| ATOM | 6819 | O | ASN | B | 715 | 30.509 | −2.602 | 110.469 | 1.00 | 58.48 | O |
| ATOM | 6820 | CB | ASN | B | 715 | 29.873 | 0.255 | 108.425 | 1.00 | 58.29 | C |
| ATOM | 6821 | CG | ASN | B | 715 | 31.113 | 0.925 | 108.997 | 1.00 | 58.20 | C |
| ATOM | 6822 | OD1 | ASN | B | 715 | 31.373 | 0.872 | 110.200 | 1.00 | 58.26 | O |
| ATOM | 6823 | ND2 | ASN | B | 715 | 31.883 | 1.568 | 108.130 | 1.00 | 58.20 | N |
| ATOM | 6824 | N | ALA | B | 716 | 30.808 | −2.433 | 108.241 | 1.00 | 58.59 | N |
| ATOM | 6825 | CA | ALA | B | 716 | 31.773 | −3.532 | 108.164 | 1.00 | 58.75 | C |
| ATOM | 6826 | C | ALA | B | 716 | 33.139 | −3.132 | 108.720 | 1.00 | 58.91 | C |
| ATOM | 6827 | O | ALA | B | 716 | 33.450 | −1.946 | 108.836 | 1.00 | 58.89 | O |
| ATOM | 6828 | CB | ALA | B | 716 | 31.908 | −4.023 | 106.733 | 1.00 | 58.73 | C |
| ATOM | 6829 | N | GLY | B | 717 | 33.947 | −4.133 | 109.064 | 1.00 | 59.14 | N |
| ATOM | 6830 | CA | GLY | B | 717 | 35.292 | −3.910 | 109.591 | 1.00 | 59.31 | C |
| ATOM | 6831 | C | GLY | B | 717 | 36.376 | −4.089 | 108.548 | 1.00 | 59.45 | C |
| ATOM | 6832 | O | GLY | B | 717 | 37.346 | −3.327 | 108.516 | 1.00 | 59.35 | O |
| ATOM | 6833 | N | GLY | B | 718 | 36.212 | −5.099 | 107.698 | 1.00 | 59.62 | N |
| ATOM | 6834 | CA | GLY | B | 718 | 37.176 | −5.382 | 106.643 | 1.00 | 60.03 | C |
| ATOM | 6835 | C | GLY | B | 718 | 38.308 | −6.287 | 107.095 | 1.00 | 60.40 | C |
| ATOM | 6836 | O | GLY | B | 718 | 38.376 | −6.646 | 108.275 | 1.00 | 60.33 | O |
| ATOM | 6837 | N | PRO | B | 719 | 39.202 | −6.675 | 106.158 | 1.00 | 60.70 | N |
| ATOM | 6838 | CA | PRO | B | 719 | 39.130 | −6.334 | 104.731 | 1.00 | 60.93 | C |
| ATOM | 6839 | C | PRO | B | 719 | 38.054 | −7.156 | 104.016 | 1.00 | 61.10 | C |
| ATOM | 6840 | O | PRO | B | 719 | 37.949 | −8.363 | 104.248 | 1.00 | 61.35 | O |
| ATOM | 6841 | CB | PRO | B | 719 | 40.526 | −6.696 | 104.213 | 1.00 | 60.96 | C |
| ATOM | 6842 | CG | PRO | B | 719 | 40.992 | −7.788 | 105.118 | 1.00 | 60.96 | C |
| ATOM | 6843 | CD | PRO | B | 719 | 40.387 | −7.501 | 106.469 | 1.00 | 60.75 | C |
| ATOM | 6844 | N | LYS | B | 720 | 37.281 | −6.497 | 103.151 | 1.00 | 61.04 | N |
| ATOM | 6845 | CA | LYS | B | 720 | 36.070 | −7.065 | 102.524 | 1.00 | 60.92 | C |
| ATOM | 6846 | C | LYS | B | 720 | 34.853 | −6.882 | 103.435 | 1.00 | 60.78 | C |
| ATOM | 6847 | O | LYS | B | 720 | 34.971 | −6.344 | 104.539 | 1.00 | 60.76 | O |
| ATOM | 6848 | CB | LYS | B | 720 | 36.240 | −8.540 | 102.126 | 1.00 | 60.94 | C |
| ATOM | 6849 | CG | LYS | B | 720 | 37.156 | −8.776 | 100.937 | 1.00 | 61.13 | C |
| ATOM | 6850 | CD | LYS | B | 720 | 37.226 | −10.253 | 100.600 | 1.00 | 61.22 | C |
| ATOM | 6851 | CE | LYS | B | 720 | 38.261 | −10.527 | 99.527 | 1.00 | 61.31 | C |
| ATOM | 6852 | NZ | LYS | B | 720 | 38.410 | −11.986 | 99.282 | 1.00 | 61.29 | N |
| ATOM | 6853 | N | ARG | B | 721 | 33.687 | −7.321 | 102.967 | 1.00 | 60.62 | N |
| ATOM | 6854 | CA | ARG | B | 721 | 32.445 | −7.133 | 103.716 | 1.00 | 60.51 | C |
| ATOM | 6855 | C | ARG | B | 721 | 32.330 | −8.107 | 104.881 | 1.00 | 60.46 | C |
| ATOM | 6856 | O | ARG | B | 721 | 31.655 | −9.132 | 104.790 | 1.00 | 60.48 | O |
| ATOM | 6857 | CB | ARG | B | 721 | 31.226 | −7.186 | 102.788 | 1.00 | 60.41 | C |
| ATOM | 6858 | CG | ARG | B | 721 | 30.940 | −5.838 | 102.154 | 1.00 | 60.07 | C |
| ATOM | 6859 | CD | ARG | B | 721 | 30.137 | −5.935 | 100.879 | 1.00 | 59.58 | C |
| ATOM | 6860 | NE | ARG | B | 721 | 30.076 | −4.631 | 100.224 | 1.00 | 59.13 | N |
| ATOM | 6861 | CZ | ARG | B | 721 | 29.298 | −4.333 | 99.189 | 1.00 | 58.61 | C |
| ATOM | 6862 | NH1 | ARG | B | 721 | 28.492 | −5.245 | 98.664 | 1.00 | 58.43 | N |
| ATOM | 6863 | NH2 | ARG | B | 721 | 29.328 | −3.110 | 98.682 | 1.00 | 58.56 | N |
| ATOM | 6864 | N | GLU | B | 722 | 32.999 | −7.756 | 105.976 | 1.00 | 60.42 | N |
| ATOM | 6865 | CA | GLU | B | 722 | 33.148 | −8.635 | 107.129 | 1.00 | 60.43 | C |
| ATOM | 6866 | C | GLU | B | 722 | 33.359 | −7.852 | 108.422 | 1.00 | 60.46 | C |
| ATOM | 6867 | O | GLU | B | 722 | 33.761 | −6.687 | 108.393 | 1.00 | 60.26 | O |
| ATOM | 6868 | CB | GLU | B | 722 | 34.322 | −9.594 | 106.910 | 1.00 | 60.38 | C |
| ATOM | 6869 | CG | GLU | B | 722 | 35.670 | −8.895 | 106.746 | 1.00 | 60.46 | C |
| ATOM | 6870 | CD | GLU | B | 722 | 36.766 | −9.826 | 106.282 | 1.00 | 60.44 | C |
| ATOM | 6871 | OE1 | GLU | B | 722 | 36.515 | −10.651 | 105.378 | 1.00 | 60.64 | O |
| ATOM | 6872 | OE2 | GLU | B | 722 | 37.890 | −9.719 | 106.811 | 1.00 | 60.27 | O |
| ATOM | 6873 | N | ASN | B | 723 | 33.091 | −8.523 | 109.545 | 1.00 | 60.59 | N |
| ATOM | 6874 | CA | ASN | B | 723 | 33.310 | −7.996 | 110.899 | 1.00 | 60.69 | C |
| ATOM | 6875 | C | ASN | B | 723 | 32.506 | −6.755 | 111.246 | 1.00 | 60.74 | C |
| ATOM | 6876 | O | ASN | B | 723 | 33.003 | −5.632 | 111.155 | 1.00 | 60.70 | O |
| ATOM | 6877 | CB | ASN | B | 723 | 34.802 | −7.785 | 111.189 | 1.00 | 60.73 | C |
| ATOM | 6878 | CG | ASN | B | 723 | 35.481 | −9.044 | 111.689 | 1.00 | 61.05 | C |
| ATOM | 6879 | OD1 | ASN | B | 723 | 35.288 | −10.133 | 111.141 | 1.00 | 61.50 | O |
| ATOM | 6880 | ND2 | ASN | B | 723 | 36.284 | −8.901 | 112.739 | 1.00 | 61.14 | N |
| ATOM | 6881 | N | GLY | B | 724 | 31.260 | −6.972 | 111.652 | 1.00 | 60.89 | N |
| ATOM | 6882 | CA | GLY | B | 724 | 30.391 | −5.890 | 112.090 | 1.00 | 61.13 | C |
| ATOM | 6883 | C | GLY | B | 724 | 31.039 | −5.090 | 113.196 | 1.00 | 61.34 | C |
| ATOM | 6884 | O | GLY | B | 724 | 31.373 | −5.630 | 114.249 | 1.00 | 61.27 | O |
| ATOM | 6885 | N | MET | B | 725 | 31.239 | −3.803 | 112.938 | 1.00 | 61.57 | N |
| ATOM | 6886 | CA | MET | B | 725 | 31.824 | −2.905 | 113.922 | 1.00 | 61.81 | C |
| ATOM | 6887 | C | MET | B | 725 | 30.804 | −1.873 | 114.366 | 1.00 | 61.78 | C |
| ATOM | 6888 | O | MET | B | 725 | 29.976 | −1.425 | 113.573 | 1.00 | 61.89 | O |
| ATOM | 6889 | CB | MET | B | 725 | 33.062 | −2.201 | 113.359 | 1.00 | 61.93 | C |
| ATOM | 6890 | CG | MET | B | 725 | 34.235 | −3.121 | 113.023 | 1.00 | 62.55 | C |
| ATOM | 6891 | SD | MET | B | 725 | 34.854 | −4.102 | 114.409 | 1.00 | 64.24 | S |
| ATOM | 6892 | CE | MET | B | 725 | 35.431 | −2.822 | 115.522 | 1.00 | 63.96 | C |
| ATOM | 6893 | N | VAL | B | 726 | 30.862 | −1.515 | 115.643 | 1.00 | 61.79 | N |
| ATOM | 6894 | CA | VAL | B | 726 | 30.067 | −0.417 | 116.173 | 1.00 | 61.79 | C |
| ATOM | 6895 | C | VAL | B | 726 | 31.008 | 0.626 | 116.775 | 1.00 | 61.78 | C |
| ATOM | 6896 | O | VAL | B | 726 | 31.727 | 0.353 | 117.741 | 1.00 | 61.78 | O |
| ATOM | 6897 | CB | VAL | B | 726 | 29.010 | −0.904 | 117.194 | 1.00 | 61.81 | C |
| ATOM | 6898 | CG1 | VAL | B | 726 | 28.343 | 0.270 | 117.888 | 1.00 | 61.82 | C |
| ATOM | 6899 | CG2 | VAL | B | 726 | 27.961 | −1.759 | 116.500 | 1.00 | 61.82 | C |
| ATOM | 6900 | N | HIS | B | 727 | 31.001 | 1.814 | 116.176 | 1.00 | 61.75 | N |
| ATOM | 6901 | CA | HIS | B | 727 | 31.914 | 2.892 | 116.543 | 1.00 | 61.70 | C |
| ATOM | 6902 | C | HIS | B | 727 | 31.177 | 3.966 | 117.335 | 1.00 | 61.65 | C |
| ATOM | 6903 | O | HIS | B | 727 | 30.006 | 4.241 | 117.069 | 1.00 | 61.62 | O |
| ATOM | 6904 | CB | HIS | B | 727 | 32.529 | 3.525 | 115.286 | 1.00 | 61.70 | C |
| ATOM | 6905 | CG | HIS | B | 727 | 33.017 | 2.536 | 114.269 | 1.00 | 61.83 | C |
| ATOM | 6906 | ND1 | HIS | B | 727 | 34.345 | 2.186 | 114.147 | 1.00 | 61.83 | N |
| ATOM | 6907 | CD2 | HIS | B | 727 | 32.358 | 1.844 | 113.309 | 1.00 | 61.77 | C |
| ATOM | 6908 | CE1 | HIS | B | 727 | 34.481 | 1.311 | 113.167 | 1.00 | 61.62 | C |
| ATOM | 6909 | NE2 | HIS | B | 727 | 33.290 | 1.087 | 112.641 | 1.00 | 61.74 | N |
| ATOM | 6910 | N | LEU | B | 728 | 31.861 | 4.572 | 118.303 | 1.00 | 61.63 | N |
| ATOM | 6911 | CA | LEU | B | 728 | 31.282 | 5.683 | 119.063 | 1.00 | 61.70 | C |
| ATOM | 6912 | C | LEU | B | 728 | 32.142 | 6.944 | 118.971 | 1.00 | 61.77 | C |
| ATOM | 6913 | O | LEU | B | 728 | 33.371 | 6.875 | 119.052 | 1.00 | 61.76 | O |
| ATOM | 6914 | CB | LEU | B | 728 | 31.039 | 5.294 | 120.529 | 1.00 | 61.71 | C |
| ATOM | 6915 | CG | LEU | B | 728 | 30.180 | 6.240 | 121.390 | 1.00 | 61.73 | C |
| ATOM | 6916 | CD1 | LEU | B | 728 | 28.694 | 6.136 | 121.053 | 1.00 | 61.40 | C |
| ATOM | 6917 | CD2 | LEU | B | 728 | 30.400 | 5.989 | 122.875 | 1.00 | 61.68 | C |
| ATOM | 6918 | N | ALA | B | 729 | 31.477 | 8.087 | 118.799 | 1.00 | 61.78 | N |
| ATOM | 6919 | CA | ALA | B | 729 | 32.139 | 9.385 | 118.687 | 1.00 | 61.82 | C |
| ATOM | 6920 | C | ALA | B | 729 | 31.570 | 10.401 | 119.673 | 1.00 | 61.88 | C |
| ATOM | 6921 | O | ALA | B | 729 | 30.369 | 10.407 | 119.949 | 1.00 | 61.81 | O |
| ATOM | 6922 | CB | ALA | B | 729 | 32.017 | 9.917 | 117.269 | 1.00 | 61.81 | C |
| ATOM | 6923 | N | ARG | B | 730 | 32.439 | 11.261 | 120.195 | 1.00 | 61.94 | N |
| ATOM | 6924 | CA | ARG | B | 730 | 32.012 | 12.344 | 121.076 | 1.00 | 62.07 | C |
| ATOM | 6925 | C | ARG | B | 730 | 31.808 | 13.614 | 120.258 | 1.00 | 61.97 | C |
| ATOM | 6926 | O | ARG | B | 730 | 32.750 | 14.154 | 119.670 | 1.00 | 61.81 | O |
| ATOM | 6927 | CB | ARG | B | 730 | 33.028 | 12.570 | 122.202 | 1.00 | 62.10 | C |
| ATOM | 6928 | CG | ARG | B | 730 | 32.488 | 13.346 | 123.404 | 1.00 | 62.57 | C |
| ATOM | 6929 | CD | ARG | B | 730 | 33.254 | 12.995 | 124.684 | 1.00 | 63.51 | C |
| ATOM | 6930 | NE | ARG | B | 730 | 34.665 | 13.388 | 124.623 | 1.00 | 64.43 | N |
| ATOM | 6931 | CZ | ARG | B | 730 | 35.649 | 12.793 | 125.296 | 1.00 | 64.75 | C |
| ATOM | 6932 | NH1 | ARG | B | 730 | 35.399 | 11.756 | 126.092 | 1.00 | 64.77 | N |
| ATOM | 6933 | NH2 | ARG | B | 730 | 36.893 | 13.233 | 125.166 | 1.00 | 64.62 | N |
| ATOM | 6934 | N | VAL | B | 731 | 30.564 | 14.075 | 120.219 | 1.00 | 61.92 | N |
| ATOM | 6935 | CA | VAL | B | 731 | 30.217 | 15.258 | 119.451 | 1.00 | 61.94 | C |
| ATOM | 6936 | C | VAL | B | 731 | 30.621 | 16.521 | 120.205 | 1.00 | 62.04 | C |
| ATOM | 6937 | O | VAL | B | 731 | 30.004 | 16.904 | 121.202 | 1.00 | 61.96 | O |
| ATOM | 6938 | CB | VAL | B | 731 | 28.716 | 15.286 | 119.076 | 1.00 | 61.93 | C |
| ATOM | 6939 | CG1 | VAL | B | 731 | 28.384 | 16.526 | 118.253 | 1.00 | 61.80 | C |
| ATOM | 6940 | CG2 | VAL | B | 731 | 28.326 | 14.019 | 118.322 | 1.00 | 61.65 | C |
| ATOM | 6941 | N | GLU | B | 732 | 31.677 | 17.154 | 119.711 | 1.00 | 62.29 | N |
| ATOM | 6942 | CA | GLU | B | 732 | 32.150 | 18.421 | 120.246 | 1.00 | 62.59 | C |
| ATOM | 6943 | C | GLU | B | 732 | 31.208 | 19.561 | 119.868 | 1.00 | 62.59 | C |
| ATOM | 6944 | O | GLU | B | 732 | 30.276 | 19.376 | 119.087 | 1.00 | 62.58 | O |
| ATOM | 6945 | CB | GLU | B | 732 | 33.572 | 18.697 | 119.764 | 1.00 | 62.67 | C |
| ATOM | 6946 | CG | GLU | B | 732 | 34.572 | 17.648 | 120.217 | 1.00 | 63.34 | C |
| ATOM | 6947 | CD | GLU | B | 732 | 35.929 | 18.239 | 120.519 | 1.00 | 64.65 | C |
| ATOM | 6948 | OE1 | GLU | B | 732 | 36.452 | 18.999 | 119.671 | 1.00 | 65.52 | O |
| ATOM | 6949 | OE2 | GLU | B | 732 | 36.469 | 17.946 | 121.608 | 1.00 | 64.38 | O |
| ATOM | 6950 | N | GLU | B | 733 | 31.459 | 20.740 | 120.420 | 1.00 | 62.71 | N |
| ATOM | 6951 | CA | GLU | B | 733 | 30.505 | 21.846 | 120.330 | 1.00 | 62.97 | C |
| ATOM | 6952 | C | GLU | B | 733 | 30.586 | 22.656 | 119.028 | 1.00 | 62.90 | C |
| ATOM | 6953 | O | GLU | B | 733 | 29.692 | 23.456 | 118.735 | 1.00 | 62.88 | O |
| ATOM | 6954 | CB | GLU | B | 733 | 30.653 | 22.762 | 121.554 | 1.00 | 62.94 | C |
| ATOM | 6955 | CG | GLU | B | 733 | 30.468 | 22.032 | 122.892 | 1.00 | 63.26 | C |
| ATOM | 6956 | CD | GLU | B | 733 | 30.498 | 22.957 | 124.103 | 1.00 | 63.31 | C |
| ATOM | 6957 | OE1 | GLU | B | 733 | 31.237 | 23.968 | 124.086 | 1.00 | 63.48 | O |
| ATOM | 6958 | OE2 | GLU | B | 733 | 29.781 | 22.659 | 125.083 | 1.00 | 63.64 | O |
| ATOM | 6959 | N | ASN | B | 734 | 31.631 | 22.415 | 118.237 | 1.00 | 62.89 | N |
| ATOM | 6960 | CA | ASN | B | 734 | 31.982 | 23.297 | 117.112 | 1.00 | 62.75 | C |
| ATOM | 6961 | C | ASN | B | 734 | 31.400 | 23.072 | 115.694 | 1.00 | 62.72 | C |
| ATOM | 6962 | O | ASN | B | 734 | 31.341 | 24.030 | 114.922 | 1.00 | 62.86 | O |
| ATOM | 6963 | CB | ASN | B | 734 | 33.504 | 23.494 | 117.033 | 1.00 | 62.76 | C |
| ATOM | 6964 | CG | ASN | B | 734 | 34.255 | 22.197 | 116.808 | 1.00 | 62.54 | C |
| ATOM | 6965 | OD1 | ASN | B | 734 | 33.923 | 21.157 | 117.381 | 1.00 | 62.40 | O |
| ATOM | 6966 | ND2 | ASN | B | 734 | 35.289 | 22.258 | 115.980 | 1.00 | 62.05 | N |
| ATOM | 6967 | N | GLY | B | 735 | 30.983 | 21.857 | 115.319 | 1.00 | 62.55 | N |
| ATOM | 6968 | CA | GLY | B | 735 | 31.012 | 20.654 | 116.142 | 1.00 | 62.34 | C |
| ATOM | 6969 | C | GLY | B | 735 | 31.604 | 19.458 | 115.420 | 1.00 | 62.23 | C |
| ATOM | 6970 | O | GLY | B | 735 | 30.898 | 18.728 | 114.718 | 1.00 | 62.11 | O |
| ATOM | 6971 | N | GLU | B | 736 | 32.909 | 19.262 | 115.596 | 1.00 | 62.12 | N |
| ATOM | 6972 | CA | GLU | B | 736 | 33.599 | 18.085 | 115.078 | 1.00 | 62.02 | C |
| ATOM | 6973 | C | GLU | B | 736 | 33.209 | 16.866 | 115.913 | 1.00 | 61.88 | C |
| ATOM | 6974 | O | GLU | B | 736 | 32.333 | 16.951 | 116.775 | 1.00 | 61.83 | O |
| ATOM | 6975 | CB | GLU | B | 736 | 35.117 | 18.294 | 115.117 | 1.00 | 62.03 | C |
| ATOM | 6976 | CG | GLU | B | 736 | 35.884 | 17.493 | 114.066 | 1.00 | 62.32 | C |
| ATOM | 6977 | CD | GLU | B | 736 | 37.321 | 17.197 | 114.468 | 1.00 | 62.41 | C |
| ATOM | 6978 | OE1 | GLU | B | 736 | 37.556 | 16.771 | 115.621 | 1.00 | 62.40 | O |
| ATOM | 6979 | OE2 | GLU | B | 736 | 38.218 | 17.378 | 113.618 | 1.00 | 62.24 | O |
| ATOM | 6980 | N | LEU | B | 737 | 33.852 | 15.734 | 115.651 | 1.00 | 61.85 | N |
| ATOM | 6981 | CA | LEU | B | 737 | 33.625 | 14.527 | 116.437 | 1.00 | 61.91 | C |
| ATOM | 6982 | C | LEU | B | 737 | 34.904 | 13.711 | 116.605 | 1.00 | 61.98 | C |
| ATOM | 6983 | O | LEU | B | 737 | 35.623 | 13.453 | 115.639 | 1.00 | 61.98 | O |
| ATOM | 6984 | CB | LEU | B | 737 | 32.485 | 13.686 | 115.846 | 1.00 | 61.91 | C |
| ATOM | 6985 | CG | LEU | B | 737 | 32.399 | 13.475 | 114.333 | 1.00 | 61.99 | C |
| ATOM | 6986 | CD1 | LEU | B | 737 | 33.036 | 12.155 | 113.935 | 1.00 | 62.32 | C |
| ATOM | 6987 | CD2 | LEU | B | 737 | 30.953 | 13.500 | 113.891 | 1.00 | 62.27 | C |
| ATOM | 6988 | N | THR | B | 738 | 35.187 | 13.334 | 117.850 | 1.00 | 62.13 | N |
| ATOM | 6989 | CA | THR | B | 738 | 36.357 | 12.519 | 118.184 | 1.00 | 62.11 | C |
| ATOM | 6990 | C | THR | B | 738 | 35.913 | 11.110 | 118.577 | 1.00 | 62.06 | C |
| ATOM | 6991 | O | THR | B | 738 | 35.059 | 10.932 | 119.452 | 1.00 | 62.02 | O |
| ATOM | 6992 | CB | THR | B | 738 | 37.211 | 13.163 | 119.312 | 1.00 | 62.12 | C |
| ATOM | 6993 | OG1 | THR | B | 738 | 36.377 | 13.481 | 120.433 | 1.00 | 61.84 | O |
| ATOM | 6994 | CG2 | THR | B | 738 | 37.893 | 14.441 | 118.814 | 1.00 | 62.01 | C |
| ATOM | 6995 | N | TRP | B | 739 | 36.486 | 10.114 | 117.910 | 1.00 | 62.02 | N |
| ATOM | 6996 | CA | TRP | B | 739 | 36.080 | 8.726 | 118.102 | 1.00 | 62.02 | C |
| ATOM | 6997 | C | TRP | B | 739 | 36.667 | 8.142 | 119.381 | 1.00 | 62.17 | C |
| ATOM | 6998 | O | TRP | B | 739 | 37.818 | 8.416 | 119.731 | 1.00 | 62.10 | O |
| ATOM | 6999 | CB | TRP | B | 739 | 36.452 | 7.885 | 116.877 | 1.00 | 61.95 | C |
| ATOM | 7000 | CG | TRP | B | 739 | 35.720 | 8.340 | 115.649 | 1.00 | 61.87 | C |
| ATOM | 7001 | CD1 | TRP | B | 739 | 36.168 | 9.218 | 114.707 | 1.00 | 61.77 | C |
| ATOM | 7002 | CD2 | TRP | B | 739 | 34.392 | 7.968 | 115.252 | 1.00 | 61.81 | C |
| ATOM | 7003 | NE1 | TRP | B | 739 | 35.210 | 9.408 | 113.739 | 1.00 | 61.71 | N |
| ATOM | 7004 | CE2 | TRP | B | 739 | 34.109 | 8.653 | 114.049 | 1.00 | 61.69 | C |
| ATOM | 7005 | CE3 | TRP | B | 739 | 33.417 | 7.115 | 115.790 | 1.00 | 61.61 | C |
| ATOM | 7006 | CZ2 | TRP | B | 739 | 32.891 | 8.514 | 113.375 | 1.00 | 61.61 | C |
| ATOM | 7007 | CZ3 | TRP | B | 739 | 32.202 | 6.982 | 115.120 | 1.00 | 61.71 | C |
| ATOM | 7008 | CH2 | TRP | B | 739 | 31.954 | 7.675 | 113.923 | 1.00 | 61.74 | C |
| ATOM | 7009 | N | LEU | B | 740 | 35.859 | 7.348 | 120.081 | 1.00 | 62.27 | N |
| ATOM | 7010 | CA | LEU | B | 740 | 36.251 | 6.793 | 121.371 | 1.00 | 62.32 | C |
| ATOM | 7011 | C | LEU | B | 740 | 36.641 | 5.323 | 121.262 | 1.00 | 62.45 | C |
| ATOM | 7012 | O | LEU | B | 740 | 37.805 | 4.970 | 121.456 | 1.00 | 62.46 | O |
| ATOM | 7013 | CB | LEU | B | 740 | 35.142 | 6.997 | 122.414 | 1.00 | 62.29 | C |
| ATOM | 7014 | CG | LEU | B | 740 | 34.636 | 8.421 | 122.703 | 1.00 | 62.17 | C |
| ATOM | 7015 | CD1 | LEU | B | 740 | 33.593 | 8.395 | 123.814 | 1.00 | 61.97 | C |
| ATOM | 7016 | CD2 | LEU | B | 740 | 35.762 | 9.391 | 123.057 | 1.00 | 61.76 | C |
| ATOM | 7017 | N | LYS | B | 741 | 35.672 | 4.472 | 120.942 | 1.00 | 62.65 | N |
| ATOM | 7018 | CA | LYS | B | 741 | 35.924 | 3.040 | 120.824 | 1.00 | 62.95 | C |
| ATOM | 7019 | C | LYS | B | 741 | 35.221 | 2.420 | 119.616 | 1.00 | 63.04 | C |
| ATOM | 7020 | O | LYS | B | 741 | 34.161 | 2.891 | 119.188 | 1.00 | 63.11 | O |
| ATOM | 7021 | CB | LYS | B | 741 | 35.535 | 2.310 | 122.117 | 1.00 | 63.00 | C |
| ATOM | 7022 | CG | LYS | B | 741 | 36.479 | 2.580 | 123.290 | 1.00 | 63.30 | C |
| ATOM | 7023 | CD | LYS | B | 741 | 36.304 | 1.575 | 124.417 | 1.00 | 63.71 | C |
| ATOM | 7024 | CE | LYS | B | 741 | 37.329 | 1.805 | 125.520 | 1.00 | 63.90 | C |
| ATOM | 7025 | NZ | LYS | B | 741 | 37.254 | 0.762 | 126.586 | 1.00 | 64.23 | N |
| ATOM | 7026 | N | HIS | B | 742 | 35.834 | 1.371 | 119.071 | 1.00 | 63.10 | N |
| ATOM | 7027 | CA | HIS | B | 742 | 35.277 | 0.615 | 117.950 | 1.00 | 63.14 | C |
| ATOM | 7028 | C | HIS | B | 742 | 35.162 | −0.852 | 118.360 | 1.00 | 63.04 | C |
| ATOM | 7029 | O | HIS | B | 742 | 36.174 | −1.539 | 118.518 | 1.00 | 63.14 | O |
| ATOM | 7030 | CB | HIS | B | 742 | 36.162 | 0.750 | 116.701 | 1.00 | 63.17 | C |
| ATOM | 7031 | CG | HIS | B | 742 | 36.831 | 2.085 | 116.566 | 1.00 | 63.59 | C |
| ATOM | 7032 | ND1 | HIS | B | 742 | 38.203 | 2.231 | 116.549 | 1.00 | 63.88 | N |
| ATOM | 7033 | CD2 | HIS | B | 742 | 36.320 | 3.334 | 116.447 | 1.00 | 63.85 | C |
| ATOM | 7034 | CE1 | HIS | B | 742 | 38.507 | 3.511 | 116.422 | 1.00 | 64.00 | C |
| ATOM | 7035 | NE2 | HIS | B | 742 | 37.382 | 4.202 | 116.358 | 1.00 | 64.10 | N |
| ATOM | 7036 | N | ASN | B | 743 | 33.931 | −1.327 | 118.531 | 1.00 | 62.92 | N |
| ATOM | 7037 | CA | ASN | B | 743 | 33.696 | −2.658 | 119.089 | 1.00 | 62.87 | C |
| ATOM | 7038 | C | ASN | B | 743 | 32.994 | −3.609 | 118.125 | 1.00 | 62.73 | C |
| ATOM | 7039 | O | ASN | B | 743 | 31.950 | −3.266 | 117.568 | 1.00 | 62.71 | O |
| ATOM | 7040 | CB | ASN | B | 743 | 32.888 | −2.567 | 120.387 | 1.00 | 62.94 | C |
| ATOM | 7041 | CG | ASN | B | 743 | 33.136 | −1.278 | 121.144 | 1.00 | 63.17 | C |
| ATOM | 7042 | OD1 | ASN | B | 743 | 32.253 | −0.425 | 121.238 | 1.00 | 63.68 | O |
| ATOM | 7043 | ND2 | ASN | B | 743 | 34.341 | −1.125 | 121.682 | 1.00 | 63.37 | N |
| ATOM | 7044 | N | PRO | B | 744 | 33.568 | −4.811 | 117.930 | 1.00 | 62.64 | N |
| ATOM | 7045 | CA | PRO | B | 744 | 32.964 | −5.838 | 117.080 | 1.00 | 62.52 | C |
| ATOM | 7046 | C | PRO | B | 744 | 31.626 | −6.329 | 117.627 | 1.00 | 62.40 | C |
| ATOM | 7047 | O | PRO | B | 744 | 31.530 | −6.730 | 118.786 | 1.00 | 62.46 | O |
| ATOM | 7048 | CB | PRO | B | 744 | 34.003 | −6.969 | 117.094 | 1.00 | 62.56 | C |
| ATOM | 7049 | CG | PRO | B | 744 | 35.280 | −6.320 | 117.532 | 1.00 | 62.53 | C |
| ATOM | 7050 | CD | PRO | B | 744 | 34.854 | −5.260 | 118.492 | 1.00 | 62.62 | C |
| ATOM | 7051 | N | ILE | B | 745 | 30.605 | −6.284 | 116.780 | 1.00 | 62.27 | N |
| ATOM | 7052 | CA | ILE | B | 745 | 29.253 | −6.694 | 117.146 | 1.00 | 62.14 | C |
| ATOM | 7053 | C | ILE | B | 745 | 28.898 | −8.057 | 116.525 | 1.00 | 61.99 | C |
| ATOM | 7054 | O | ILE | B | 745 | 28.021 | −8.771 | 117.020 | 1.00 | 62.01 | O |
| ATOM | 7055 | CB | ILE | B | 745 | 28.213 | −5.590 | 116.761 | 1.00 | 62.12 | C |
| ATOM | 7056 | CG1 | ILE | B | 745 | 26.813 | −5.933 | 117.283 | 1.00 | 62.17 | C |
| ATOM | 7057 | CG2 | ILE | B | 745 | 28.213 | −5.315 | 115.249 | 1.00 | 62.19 | C |
| ATOM | 7058 | CD1 | ILE | B | 745 | 25.782 | −4.863 | 117.018 | 1.00 | 62.19 | C |
| ATOM | 7059 | N | GLN | B | 746 | 29.601 | −8.407 | 115.450 | 1.00 | 61.84 | N |
| ATOM | 7060 | CA | GLN | B | 746 | 29.360 | −9.639 | 114.709 | 1.00 | 61.70 | C |
| ATOM | 7061 | C | GLN | B | 746 | 30.625 | −10.030 | 113.952 | 1.00 | 61.63 | C |
| ATOM | 7062 | O | GLN | B | 746 | 31.306 | −9.173 | 113.385 | 1.00 | 61.63 | O |
| ATOM | 7063 | CB | GLN | B | 746 | 28.185 | −9.452 | 113.737 | 1.00 | 61.70 | C |
| ATOM | 7064 | CG | GLN | B | 746 | 27.910 | −10.621 | 112.793 | 1.00 | 61.73 | C |
| ATOM | 7065 | CD | GLN | B | 746 | 27.292 | −11.822 | 113.487 | 1.00 | 62.13 | C |
| ATOM | 7066 | OE1 | GLN | B | 746 | 26.141 | −11.781 | 113.924 | 1.00 | 62.00 | O |
| ATOM | 7067 | NE2 | GLN | B | 746 | 28.053 | −12.907 | 113.577 | 1.00 | 62.17 | N |
| ATOM | 7068 | N | LYS | B | 747 | 30.939 | −11.323 | 113.969 | 1.00 | 61.51 | N |
| ATOM | 7069 | CA | LYS | B | 747 | 32.035 | −11.877 | 113.179 | 1.00 | 61.33 | C |
| ATOM | 7070 | C | LYS | B | 747 | 31.463 | −12.577 | 111.951 | 1.00 | 61.06 | C |
| ATOM | 7071 | O | LYS | B | 747 | 30.262 | −12.841 | 111.887 | 1.00 | 60.90 | O |
| ATOM | 7072 | CB | LYS | B | 747 | 32.875 | −12.851 | 114.011 | 1.00 | 61.46 | C |
| ATOM | 7073 | CG | LYS | B | 747 | 33.632 | −12.196 | 115.156 | 1.00 | 61.81 | C |
| ATOM | 7074 | CD | LYS | B | 747 | 34.425 | −13.215 | 115.951 | 1.00 | 62.37 | C |
| ATOM | 7075 | CE | LYS | B | 747 | 35.035 | −12.588 | 117.192 | 1.00 | 62.40 | C |
| ATOM | 7076 | NZ | LYS | B | 747 | 35.907 | −13.551 | 117.918 | 1.00 | 62.72 | N |
| ATOM | 7077 | N | GLY | B | 748 | 32.327 | −12.871 | 110.982 | 1.00 | 60.85 | N |
| ATOM | 7078 | CA | GLY | B | 748 | 31.896 | −13.425 | 109.699 | 1.00 | 60.66 | C |
| ATOM | 7079 | C | GLY | B | 748 | 31.683 | −12.320 | 108.682 | 1.00 | 60.43 | C |
| ATOM | 7080 | O | GLY | B | 748 | 32.087 | −11.176 | 108.912 | 1.00 | 60.36 | O |
| ATOM | 7081 | N | GLU | B | 749 | 31.050 | −12.649 | 107.557 | 1.00 | 60.16 | N |
| ATOM | 7082 | CA | GLU | B | 749 | 30.782 | −11.631 | 106.544 | 1.00 | 59.97 | C |
| ATOM | 7083 | C | GLU | B | 749 | 29.573 | −10.755 | 106.906 | 1.00 | 59.50 | C |
| ATOM | 7084 | O | GLU | B | 749 | 28.508 | −11.259 | 107.261 | 1.00 | 59.47 | O |
| ATOM | 7085 | CB | GLU | B | 749 | 30.726 | −12.211 | 105.116 | 1.00 | 59.97 | C |
| ATOM | 7086 | CG | GLU | B | 749 | 29.544 | −13.110 | 104.763 | 1.00 | 60.25 | C |
| ATOM | 7087 | CD | GLU | B | 749 | 29.384 | −13.292 | 103.247 | 1.00 | 60.47 | C |
| ATOM | 7088 | OE1 | GLU | B | 749 | 30.031 | −12.550 | 102.472 | 1.00 | 60.77 | O |
| ATOM | 7089 | OE2 | GLU | B | 749 | 28.606 | −14.175 | 102.822 | 1.00 | 61.35 | O |
| ATOM | 7090 | N | PHE | B | 750 | 29.775 | −9.441 | 106.820 | 1.00 | 59.03 | N |
| ATOM | 7091 | CA | PHE | B | 750 | 28.840 | −8.435 | 107.332 | 1.00 | 58.65 | C |
| ATOM | 7092 | C | PHE | B | 750 | 28.756 | −7.238 | 106.372 | 1.00 | 58.34 | C |
| ATOM | 7093 | O | PHE | B | 750 | 29.787 | −6.752 | 105.893 | 1.00 | 58.32 | O |
| ATOM | 7094 | CB | PHE | B | 750 | 29.314 | −7.979 | 108.717 | 1.00 | 58.60 | C |
| ATOM | 7095 | CG | PHE | B | 750 | 28.491 | −6.876 | 109.324 | 1.00 | 58.78 | C |
| ATOM | 7096 | CD1 | PHE | B | 750 | 27.502 | −7.167 | 110.257 | 1.00 | 59.05 | C |
| ATOM | 7097 | CD2 | PHE | B | 750 | 28.722 | −5.542 | 108.988 | 1.00 | 58.81 | C |
| ATOM | 7098 | CE1 | PHE | B | 750 | 26.748 | −6.151 | 110.837 | 1.00 | 58.91 | C |
| ATOM | 7099 | CE2 | PHE | B | 750 | 27.968 | −4.523 | 109.556 | 1.00 | 58.72 | C |
| ATOM | 7100 | CZ | PHE | B | 750 | 26.980 | −4.828 | 110.483 | 1.00 | 58.75 | C |
| ATOM | 7101 | N | ALA | B | 751 | 27.541 | −6.751 | 106.112 | 1.00 | 57.83 | N |
| ATOM | 7102 | CA | ALA | B | 751 | 27.334 | −5.688 | 105.114 | 1.00 | 57.47 | C |
| ATOM | 7103 | C | ALA | B | 751 | 26.444 | −4.510 | 105.560 | 1.00 | 57.17 | C |
| ATOM | 7104 | O | ALA | B | 751 | 26.910 | −3.601 | 106.255 | 1.00 | 57.03 | O |
| ATOM | 7105 | CB | ALA | B | 751 | 26.834 | −6.287 | 103.793 | 1.00 | 57.36 | C |
| ATOM | 7106 | N | TYR | B | 752 | 25.179 | −4.529 | 105.139 | 1.00 | 56.95 | N |
| ATOM | 7107 | CA | TYR | B | 752 | 24.235 | −3.437 | 105.401 | 1.00 | 56.77 | C |
| ATOM | 7108 | C | TYR | B | 752 | 23.599 | −3.571 | 106.782 | 1.00 | 56.62 | C |
| ATOM | 7109 | O | TYR | B | 752 | 23.334 | −4.686 | 107.245 | 1.00 | 56.38 | O |
| ATOM | 7110 | CB | TYR | B | 752 | 23.127 | −3.408 | 104.338 | 1.00 | 56.74 | C |
| ATOM | 7111 | CG | TYR | B | 752 | 23.494 | −2.810 | 102.992 | 1.00 | 56.84 | C |
| ATOM | 7112 | CD1 | TYR | B | 752 | 22.607 | −1.957 | 102.329 | 1.00 | 56.71 | C |
| ATOM | 7113 | CD2 | TYR | B | 752 | 24.712 | −3.106 | 102.369 | 1.00 | 57.10 | C |
| ATOM | 7114 | CE1 | TYR | B | 752 | 22.924 | −1.408 | 101.088 | 1.00 | 56.47 | C |
| ATOM | 7115 | CE2 | TYR | B | 752 | 25.041 | −2.556 | 101.129 | 1.00 | 57.01 | C |
| ATOM | 7116 | CZ | TYR | B | 752 | 24.141 | −1.711 | 100.496 | 1.00 | 56.86 | C |
| ATOM | 7117 | OH | TYR | B | 752 | 24.462 | −1.170 | 99.273 | 1.00 | 56.72 | O |
| ATOM | 7118 | N | ASN | B | 753 | 23.336 | −2.430 | 107.419 | 1.00 | 56.39 | N |
| ATOM | 7119 | CA | ASN | B | 753 | 22.765 | −2.396 | 108.768 | 1.00 | 56.22 | C |
| ATOM | 7120 | C | ASN | B | 753 | 21.921 | −1.150 | 109.040 | 1.00 | 56.26 | C |
| ATOM | 7121 | O | ASN | B | 753 | 22.049 | −0.142 | 108.344 | 1.00 | 56.10 | O |
| ATOM | 7122 | CB | ASN | B | 753 | 23.871 | −2.519 | 109.821 | 1.00 | 56.00 | C |
| ATOM | 7123 | CG | ASN | B | 753 | 24.824 | −1.343 | 109.805 | 1.00 | 55.60 | C |
| ATOM | 7124 | OD1 | ASN | B | 753 | 24.660 | −0.398 | 110.572 | 1.00 | 54.87 | O |
| ATOM | 7125 | ND2 | ASN | B | 753 | 25.822 | −1.391 | 108.918 | 1.00 | 55.35 | N |
| ATOM | 7126 | N | SER | B | 754 | 21.069 | −1.231 | 110.063 | 1.00 | 56.41 | N |
| ATOM | 7127 | CA | SER | B | 754 | 20.231 | −0.106 | 110.479 | 1.00 | 56.53 | C |
| ATOM | 7128 | C | SER | B | 754 | 20.116 | −0.019 | 112.007 | 1.00 | 56.77 | C |
| ATOM | 7129 | O | SER | B | 754 | 19.758 | −0.996 | 112.664 | 1.00 | 56.83 | O |
| ATOM | 7130 | CB | SER | B | 754 | 18.847 | −0.210 | 109.836 | 1.00 | 56.40 | C |
| ATOM | 7131 | OG | SER | B | 754 | 18.108 | 0.984 | 110.018 | 1.00 | 56.22 | O |
| ATOM | 7132 | N | LEU | B | 755 | 20.421 | 1.156 | 112.558 | 1.00 | 57.02 | N |
| ATOM | 7133 | CA | LEU | B | 755 | 20.419 | 1.379 | 114.008 | 1.00 | 57.23 | C |
| ATOM | 7134 | C | LEU | B | 755 | 19.213 | 2.184 | 114.490 | 1.00 | 57.55 | C |
| ATOM | 7135 | O | LEU | B | 755 | 18.723 | 3.068 | 113.788 | 1.00 | 57.64 | O |
| ATOM | 7136 | CB | LEU | B | 755 | 21.689 | 2.120 | 114.435 | 1.00 | 57.13 | C |
| ATOM | 7137 | CG | LEU | B | 755 | 23.074 | 1.488 | 114.304 | 1.00 | 56.88 | C |
| ATOM | 7138 | CD1 | LEU | B | 755 | 24.131 | 2.574 | 114.389 | 1.00 | 56.42 | C |
| ATOM | 7139 | CD2 | LEU | B | 755 | 23.306 | 0.440 | 115.377 | 1.00 | 56.80 | C |
| ATOM | 7140 | N | GLN | B | 756 | 18.754 | 1.883 | 115.701 | 1.00 | 57.92 | N |
| ATOM | 7141 | CA | GLN | B | 756 | 17.712 | 2.669 | 116.361 | 1.00 | 58.32 | C |
| ATOM | 7142 | C | GLN | B | 756 | 18.018 | 2.870 | 117.847 | 1.00 | 58.83 | C |
| ATOM | 7143 | O | GLN | B | 756 | 18.878 | 2.189 | 118.411 | 1.00 | 58.95 | O |
| ATOM | 7144 | CB | GLN | B | 756 | 16.335 | 2.016 | 116.186 | 1.00 | 58.21 | C |
| ATOM | 7145 | CG | GLN | B | 756 | 15.635 | 2.357 | 114.876 | 1.00 | 57.48 | C |
| ATOM | 7146 | CD | GLN | B | 756 | 15.135 | 3.794 | 114.818 | 1.00 | 56.35 | C |
| ATOM | 7147 | OE1 | GLN | B | 756 | 14.191 | 4.168 | 115.511 | 1.00 | 55.68 | O |
| ATOM | 7148 | NE2 | GLN | B | 756 | 15.765 | 4.603 | 113.976 | 1.00 | 55.89 | N |
| ATOM | 7149 | N | GLU | B | 757 | 17.313 | 3.812 | 118.468 | 1.00 | 59.34 | N |
| ATOM | 7150 | CA | GLU | B | 757 | 17.431 | 4.056 | 119.900 | 1.00 | 59.81 | C |
| ATOM | 7151 | C | GLU | B | 757 | 16.196 | 3.488 | 120.593 | 1.00 | 60.24 | C |
| ATOM | 7152 | O | GLU | B | 757 | 15.075 | 3.942 | 120.346 | 1.00 | 60.28 | O |
| ATOM | 7153 | CB | GLU | B | 757 | 17.575 | 5.555 | 120.168 | 1.00 | 59.77 | C |
| ATOM | 7154 | CG | GLU | B | 757 | 18.000 | 5.912 | 121.582 | 1.00 | 59.87 | C |
| ATOM | 7155 | CD | GLU | B | 757 | 18.594 | 7.310 | 121.691 | 1.00 | 59.84 | C |
| ATOM | 7156 | OE1 | GLU | B | 757 | 18.462 | 8.112 | 120.741 | 1.00 | 59.98 | O |
| ATOM | 7157 | OE2 | GLU | B | 757 | 19.202 | 7.607 | 122.737 | 1.00 | 59.50 | O |
| ATOM | 7158 | N | LEU | B | 758 | 16.406 | 2.490 | 121.452 | 1.00 | 60.77 | N |
| ATOM | 7159 | CA | LEU | B | 758 | 15.301 | 1.778 | 122.104 | 1.00 | 61.20 | C |
| ATOM | 7160 | C | LEU | B | 758 | 14.868 | 2.397 | 123.433 | 1.00 | 61.45 | C |
| ATOM | 7161 | O | LEU | B | 758 | 13.864 | 1.984 | 124.014 | 1.00 | 61.53 | O |
| ATOM | 7162 | CB | LEU | B | 758 | 15.644 | 0.292 | 122.301 | 1.00 | 61.21 | C |
| ATOM | 7163 | CG | LEU | B | 758 | 16.190 | −0.545 | 121.134 | 1.00 | 61.35 | C |
| ATOM | 7164 | CD1 | LEU | B | 758 | 16.378 | −1.988 | 121.573 | 1.00 | 61.47 | C |
| ATOM | 7165 | CD2 | LEU | B | 758 | 15.306 | −0.481 | 119.893 | 1.00 | 61.11 | C |
| ATOM | 7166 | N | GLY | B | 759 | 15.617 | 3.394 | 123.902 | 1.00 | 61.82 | N |
| ATOM | 7167 | CA | GLY | B | 759 | 15.363 | 4.007 | 125.203 | 1.00 | 62.18 | C |
| ATOM | 7168 | C | GLY | B | 759 | 16.062 | 3.233 | 126.304 | 1.00 | 62.50 | C |
| ATOM | 7169 | O | GLY | B | 759 | 16.580 | 2.140 | 126.063 | 1.00 | 62.53 | O |
| ATOM | 7170 | N | ASN | B | 760 | 16.070 | 3.798 | 127.513 | 1.00 | 62.82 | N |
| ATOM | 7171 | CA | ASN | B | 760 | 16.780 | 3.221 | 128.671 | 1.00 | 63.15 | C |
| ATOM | 7172 | C | ASN | B | 760 | 18.275 | 3.006 | 128.413 | 1.00 | 63.30 | C |
| ATOM | 7173 | O | ASN | B | 760 | 18.907 | 2.147 | 129.034 | 1.00 | 63.37 | O |
| ATOM | 7174 | CB | ASN | B | 760 | 16.128 | 1.905 | 129.134 | 1.00 | 63.15 | C |
| ATOM | 7175 | CG | ASN | B | 760 | 14.796 | 2.120 | 129.831 | 1.00 | 63.39 | C |
| ATOM | 7176 | OD1 | ASN | B | 760 | 14.666 | 1.870 | 131.031 | 1.00 | 63.45 | O |
| ATOM | 7177 | ND2 | ASN | B | 760 | 13.796 | 2.580 | 129.082 | 1.00 | 63.36 | N |
| ATOM | 7178 | N | GLY | B | 761 | 18.830 | 3.795 | 127.494 | 1.00 | 63.48 | N |
| ATOM | 7179 | CA | GLY | B | 761 | 20.204 | 3.612 | 127.043 | 1.00 | 63.69 | C |
| ATOM | 7180 | C | GLY | B | 761 | 20.404 | 2.296 | 126.310 | 1.00 | 63.83 | C |
| ATOM | 7181 | O | GLY | B | 761 | 21.493 | 1.726 | 126.347 | 1.00 | 63.79 | O |
| ATOM | 7182 | N | GLU | B | 762 | 19.347 | 1.813 | 125.655 | 1.00 | 63.98 | N |
| ATOM | 7183 | CA | GLU | B | 762 | 19.411 | 0.591 | 124.846 | 1.00 | 64.12 | C |
| ATOM | 7184 | C | GLU | B | 762 | 19.232 | 0.880 | 123.362 | 1.00 | 64.15 | C |
| ATOM | 7185 | O | GLU | B | 762 | 18.469 | 1.770 | 122.984 | 1.00 | 64.19 | O |
| ATOM | 7186 | CB | GLU | B | 762 | 18.365 | −0.427 | 125.298 | 1.00 | 64.14 | C |
| ATOM | 7187 | CG | GLU | B | 762 | 18.866 | −1.419 | 126.331 | 1.00 | 64.25 | C |
| ATOM | 7188 | CD | GLU | B | 762 | 17.976 | −2.644 | 126.447 | 1.00 | 64.53 | C |
| ATOM | 7189 | OE1 | GLU | B | 762 | 17.154 | −2.886 | 125.536 | 1.00 | 64.62 | O |
| ATOM | 7190 | OE2 | GLU | B | 762 | 18.103 | −3.371 | 127.452 | 1.00 | 64.48 | O |
| ATOM | 7191 | N | TYR | B | 763 | 19.938 | 0.115 | 122.531 | 1.00 | 64.22 | N |
| ATOM | 7192 | CA | TYR | B | 763 | 19.920 | 0.301 | 121.081 | 1.00 | 64.20 | C |
| ATOM | 7193 | C | TYR | B | 763 | 19.697 | −1.020 | 120.348 | 1.00 | 64.27 | C |
| ATOM | 7194 | O | TYR | B | 763 | 20.063 | −2.085 | 120.848 | 1.00 | 64.30 | O |
| ATOM | 7195 | CB | TYR | B | 763 | 21.218 | 0.968 | 120.608 | 1.00 | 64.12 | C |
| ATOM | 7196 | CG | TYR | B | 763 | 21.621 | 2.170 | 121.437 | 1.00 | 64.09 | C |
| ATOM | 7197 | CD1 | TYR | B | 763 | 22.707 | 2.108 | 122.307 | 1.00 | 64.05 | C |
| ATOM | 7198 | CD2 | TYR | B | 763 | 20.900 | 3.362 | 121.367 | 1.00 | 64.28 | C |
| ATOM | 7199 | CE1 | TYR | B | 763 | 23.074 | 3.206 | 123.080 | 1.00 | 64.06 | C |
| ATOM | 7200 | CE2 | TYR | B | 763 | 21.258 | 4.465 | 122.136 | 1.00 | 64.42 | C |
| ATOM | 7201 | CZ | TYR | B | 763 | 22.343 | 4.381 | 122.988 | 1.00 | 64.12 | C |
| ATOM | 7202 | OH | TYR | B | 763 | 22.693 | 5.472 | 123.746 | 1.00 | 63.98 | O |
| ATOM | 7203 | N | GLY | B | 764 | 19.085 | −0.937 | 119.169 | 1.00 | 64.33 | N |
| ATOM | 7204 | CA | GLY | B | 764 | 18.817 | −2.112 | 118.338 | 1.00 | 64.42 | C |
| ATOM | 7205 | C | GLY | B | 764 | 19.419 | −1.999 | 116.949 | 1.00 | 64.48 | C |
| ATOM | 7206 | O | GLY | B | 764 | 19.555 | −0.899 | 116.408 | 1.00 | 64.51 | O |
| ATOM | 7207 | N | ILE | B | 765 | 19.777 | −3.141 | 116.367 | 1.00 | 64.53 | N |
| ATOM | 7208 | CA | ILE | B | 765 | 20.397 | −3.167 | 115.041 | 1.00 | 64.59 | C |
| ATOM | 7209 | C | ILE | B | 765 | 20.001 | −4.384 | 114.203 | 1.00 | 64.79 | C |
| ATOM | 7210 | O | ILE | B | 765 | 20.290 | −5.527 | 114.568 | 1.00 | 64.78 | O |
| ATOM | 7211 | CB | ILE | B | 765 | 21.950 | −3.038 | 115.121 | 1.00 | 64.56 | C |
| ATOM | 7212 | CG1 | ILE | B | 765 | 22.589 | −3.210 | 113.734 | 1.00 | 64.43 | C |
| ATOM | 7213 | CG2 | ILE | B | 765 | 22.524 | −4.023 | 116.133 | 1.00 | 64.50 | C |
| ATOM | 7214 | CD1 | ILE | B | 765 | 24.053 | −2.817 | 113.654 | 1.00 | 64.40 | C |
| ATOM | 7215 | N | LEU | B | 766 | 19.328 | −4.122 | 113.084 | 1.00 | 65.03 | N |
| ATOM | 7216 | CA | LEU | B | 766 | 19.101 | −5.138 | 112.059 | 1.00 | 65.20 | C |
| ATOM | 7217 | C | LEU | B | 766 | 20.224 | −5.047 | 111.031 | 1.00 | 65.40 | C |
| ATOM | 7218 | O | LEU | B | 766 | 20.543 | −3.957 | 110.547 | 1.00 | 65.47 | O |
| ATOM | 7219 | CB | LEU | B | 766 | 17.737 | −4.946 | 111.390 | 1.00 | 65.16 | C |
| ATOM | 7220 | CG | LEU | B | 766 | 17.366 | −5.866 | 110.218 | 1.00 | 65.21 | C |
| ATOM | 7221 | CD1 | LEU | B | 766 | 17.208 | −7.322 | 110.651 | 1.00 | 65.18 | C |
| ATOM | 7222 | CD2 | LEU | B | 766 | 16.102 | −5.368 | 109.524 | 1.00 | 65.21 | C |
| ATOM | 7223 | N | TYR | B | 767 | 20.820 | −6.192 | 110.706 | 1.00 | 65.60 | N |
| ATOM | 7224 | CA | TYR | B | 767 | 21.995 | −6.227 | 109.833 | 1.00 | 65.85 | C |
| ATOM | 7225 | C | TYR | B | 767 | 22.017 | −7.403 | 108.851 | 1.00 | 65.87 | C |
| ATOM | 7226 | O | TYR | B | 767 | 21.142 | −8.264 | 108.883 | 1.00 | 65.88 | O |
| ATOM | 7227 | CB | TYR | B | 767 | 23.289 | −6.176 | 110.660 | 1.00 | 65.95 | C |
| ATOM | 7228 | CG | TYR | B | 767 | 23.463 | −7.297 | 111.672 | 1.00 | 66.30 | C |
| ATOM | 7229 | CD1 | TYR | B | 767 | 24.076 | −8.501 | 111.313 | 1.00 | 66.37 | C |
| ATOM | 7230 | CD2 | TYR | B | 767 | 23.041 | −7.142 | 112.996 | 1.00 | 66.34 | C |
| ATOM | 7231 | CE1 | TYR | B | 767 | 24.248 | −9.528 | 112.238 | 1.00 | 66.42 | C |
| ATOM | 7232 | CE2 | TYR | B | 767 | 23.209 | −8.163 | 113.929 | 1.00 | 66.35 | C |
| ATOM | 7233 | CZ | TYR | B | 767 | 23.813 | −9.353 | 113.543 | 1.00 | 66.49 | C |
| ATOM | 7234 | OH | TYR | B | 767 | 23.981 | −10.369 | 114.458 | 1.00 | 66.48 | O |
| ATOM | 7235 | N | GLU | B | 768 | 23.026 | −7.419 | 107.984 | 1.00 | 65.95 | N |
| ATOM | 7236 | CA | GLU | B | 768 | 23.182 | −8.449 | 106.959 | 1.00 | 66.09 | C |
| ATOM | 7237 | C | GLU | B | 768 | 24.312 | −9.413 | 107.318 | 1.00 | 66.34 | C |
| ATOM | 7238 | O | GLU | B | 768 | 25.435 | −8.981 | 107.596 | 1.00 | 66.30 | O |
| ATOM | 7239 | CB | GLU | B | 768 | 23.470 | −7.800 | 105.602 | 1.00 | 66.09 | C |
| ATOM | 7240 | CG | GLU | B | 768 | 22.288 | −7.077 | 104.978 | 1.00 | 65.78 | C |
| ATOM | 7241 | CD | GLU | B | 768 | 22.523 | −6.708 | 103.524 | 1.00 | 65.81 | C |
| ATOM | 7242 | OE1 | GLU | B | 768 | 23.671 | −6.378 | 103.162 | 1.00 | 65.19 | O |
| ATOM | 7243 | OE2 | GLU | B | 768 | 21.555 | −6.745 | 102.738 | 1.00 | 65.61 | O |
| ATOM | 7244 | N | HIS | B | 769 | 24.016 | −10.714 | 107.303 | 1.00 | 66.63 | N |
| ATOM | 7245 | CA | HIS | B | 769 | 24.998 | −11.737 | 107.685 | 1.00 | 66.92 | C |
| ATOM | 7246 | C | HIS | B | 769 | 24.750 | −13.100 | 107.033 | 1.00 | 66.98 | C |
| ATOM | 7247 | O | HIS | B | 769 | 23.617 | −13.430 | 106.672 | 1.00 | 67.03 | O |
| ATOM | 7248 | CB | HIS | B | 769 | 25.047 | −11.885 | 109.212 | 1.00 | 67.04 | C |
| ATOM | 7249 | CG | HIS | B | 769 | 26.175 | −12.739 | 109.698 | 1.00 | 67.46 | C |
| ATOM | 7250 | ND1 | HIS | B | 769 | 25.996 | −14.039 | 110.120 | 1.00 | 68.01 | N |
| ATOM | 7251 | CD2 | HIS | B | 769 | 27.501 | −12.486 | 109.811 | 1.00 | 67.70 | C |
| ATOM | 7252 | CE1 | HIS | B | 769 | 27.161 | −14.546 | 110.483 | 1.00 | 68.23 | C |
| ATOM | 7253 | NE2 | HIS | B | 769 | 28.091 | −13.624 | 110.303 | 1.00 | 67.93 | N |
| ATOM | 7254 | N | THR | B | 770 | 25.823 | −13.881 | 106.898 | 1.00 | 67.09 | N |
| ATOM | 7255 | CA | THR | B | 770 | 25.770 | −15.231 | 106.336 | 1.00 | 67.24 | C |
| ATOM | 7256 | C | THR | B | 770 | 26.564 | −16.236 | 107.171 | 1.00 | 67.28 | C |
| ATOM | 7257 | O | THR | B | 770 | 27.663 | −15.937 | 107.643 | 1.00 | 67.17 | O |
| ATOM | 7258 | CB | THR | B | 770 | 26.268 | −15.248 | 104.868 | 1.00 | 67.31 | C |
| ATOM | 7259 | OG1 | THR | B | 770 | 25.190 | −14.885 | 104.000 | 1.00 | 67.62 | O |
| ATOM | 7260 | CG2 | THR | B | 770 | 26.796 | −16.630 | 104.451 | 1.00 | 67.37 | C |
| ATOM | 7261 | N | GLU | B | 771 | 25.981 | −17.421 | 107.342 | 1.00 | 67.36 | N |
| ATOM | 7262 | CA | GLU | B | 771 | 26.638 | −18.565 | 107.977 | 1.00 | 67.43 | C |
| ATOM | 7263 | C | GLU | B | 771 | 25.866 | −19.844 | 107.660 | 1.00 | 67.44 | C |
| ATOM | 7264 | O | GLU | B | 771 | 24.675 | −19.791 | 107.368 | 1.00 | 67.40 | O |
| ATOM | 7265 | CB | GLU | B | 771 | 26.758 | −18.370 | 109.494 | 1.00 | 67.47 | C |
| ATOM | 7266 | CG | GLU | B | 771 | 25.434 | −18.155 | 110.227 | 1.00 | 67.46 | C |
| ATOM | 7267 | CD | GLU | B | 771 | 25.610 | −17.981 | 111.727 | 1.00 | 67.54 | C |
| ATOM | 7268 | OE1 | GLU | B | 771 | 26.764 | −18.018 | 112.209 | 1.00 | 67.34 | O |
| ATOM | 7269 | OE2 | GLU | B | 771 | 24.588 | −17.806 | 112.426 | 1.00 | 67.81 | O |
| ATOM | 7270 | N | LYS | B | 772 | 26.547 | −20.987 | 107.727 | 1.00 | 67.52 | N |
| ATOM | 7271 | CA | LYS | B | 772 | 25.954 | −22.294 | 107.402 | 1.00 | 67.55 | C |
| ATOM | 7272 | C | LYS | B | 772 | 25.457 | −22.367 | 105.949 | 1.00 | 67.57 | C |
| ATOM | 7273 | O | LYS | B | 772 | 24.386 | −22.923 | 105.670 | 1.00 | 67.62 | O |
| ATOM | 7274 | CB | LYS | B | 772 | 24.831 | −22.662 | 108.388 | 1.00 | 67.52 | C |
| ATOM | 7275 | CG | LYS | B | 772 | 25.280 | −22.836 | 109.840 | 1.00 | 67.61 | C |
| ATOM | 7276 | CD | LYS | B | 772 | 24.091 | −23.050 | 110.779 | 1.00 | 67.59 | C |
| ATOM | 7277 | CE | LYS | B | 772 | 23.268 | −21.771 | 110.957 | 1.00 | 67.57 | C |
| ATOM | 7278 | NZ | LYS | B | 772 | 22.052 | −21.991 | 111.784 | 1.00 | 67.45 | N |
| ATOM | 7279 | N | GLY | B | 773 | 26.246 | −21.798 | 105.036 | 1.00 | 67.52 | N |
| ATOM | 7280 | CA | GLY | B | 773 | 25.943 | −21.804 | 103.602 | 1.00 | 67.49 | C |
| ATOM | 7281 | C | GLY | B | 773 | 24.698 | −21.030 | 103.205 | 1.00 | 67.49 | C |
| ATOM | 7282 | O | GLY | B | 773 | 24.028 | −21.380 | 102.231 | 1.00 | 67.37 | O |
| ATOM | 7283 | N | GLN | B | 774 | 24.393 | −19.974 | 103.957 | 1.00 | 67.58 | N |
| ATOM | 7284 | CA | GLN | B | 774 | 23.202 | −19.159 | 103.709 | 1.00 | 67.69 | C |
| ATOM | 7285 | C | GLN | B | 774 | 23.357 | −18.253 | 102.483 | 1.00 | 67.72 | C |
| ATOM | 7286 | O | GLN | B | 774 | 24.428 | −17.686 | 102.242 | 1.00 | 67.66 | O |
| ATOM | 7287 | CB | GLN | B | 774 | 22.824 | −18.351 | 104.956 | 1.00 | 67.67 | C |
| ATOM | 7288 | CG | GLN | B | 774 | 22.092 | −19.168 | 106.027 | 1.00 | 67.65 | C |
| ATOM | 7289 | CD | GLN | B | 774 | 21.957 | −18.439 | 107.361 | 1.00 | 67.65 | C |
| ATOM | 7290 | OE1 | GLN | B | 774 | 22.808 | −17.629 | 107.738 | 1.00 | 67.84 | O |
| ATOM | 7291 | NE2 | GLN | B | 774 | 20.884 | −18.735 | 108.085 | 1.00 | 67.41 | N |
| ATOM | 7292 | N | ASN | B | 775 | 22.266 | −18.123 | 101.728 | 1.00 | 67.77 | N |
| ATOM | 7293 | CA | ASN | B | 775 | 22.247 | −17.447 | 100.424 | 1.00 | 67.84 | C |
| ATOM | 7294 | C | ASN | B | 775 | 22.668 | −15.976 | 100.392 | 1.00 | 67.98 | C |
| ATOM | 7295 | O | ASN | B | 775 | 23.041 | −15.467 | 99.330 | 1.00 | 68.04 | O |
| ATOM | 7296 | CB | ASN | B | 775 | 20.868 | −17.600 | 99.773 | 1.00 | 67.75 | C |
| ATOM | 7297 | CG | ASN | B | 775 | 20.659 | −18.969 | 99.152 | 1.00 | 67.52 | C |
| ATOM | 7298 | OD1 | ASN | B | 775 | 21.604 | −19.733 | 98.957 | 1.00 | 67.27 | O |
| ATOM | 7299 | ND2 | ASN | B | 775 | 19.412 | −19.280 | 98.828 | 1.00 | 67.59 | N |
| ATOM | 7300 | N | ALA | B | 776 | 22.599 | −15.307 | 101.545 | 1.00 | 68.12 | N |
| ATOM | 7301 | CA | ALA | B | 776 | 22.960 | −13.885 | 101.698 | 1.00 | 68.25 | C |
| ATOM | 7302 | C | ALA | B | 776 | 21.870 | −12.925 | 101.214 | 1.00 | 68.37 | C |
| ATOM | 7303 | O | ALA | B | 776 | 21.588 | −12.843 | 100.017 | 1.00 | 68.27 | O |
| ATOM | 7304 | CB | ALA | B | 776 | 24.313 | −13.562 | 101.038 | 1.00 | 68.21 | C |
| ATOM | 7305 | N | TYR | B | 777 | 21.258 | −12.198 | 102.147 | 1.00 | 68.60 | N |
| ATOM | 7306 | CA | TYR | B | 777 | 21.594 | −12.283 | 103.568 | 1.00 | 68.89 | C |
| ATOM | 7307 | C | TYR | B | 777 | 20.402 | −12.754 | 104.404 | 1.00 | 69.29 | C |
| ATOM | 7308 | O | TYR | B | 777 | 19.283 | −12.857 | 103.900 | 1.00 | 69.23 | O |
| ATOM | 7309 | CB | TYR | B | 777 | 22.112 | −10.934 | 104.075 | 1.00 | 68.75 | C |
| ATOM | 7310 | CG | TYR | B | 777 | 23.424 | −10.492 | 103.458 | 1.00 | 68.51 | C |
| ATOM | 7311 | CD1 | TYR | B | 777 | 23.450 | −9.741 | 102.282 | 1.00 | 68.46 | C |
| ATOM | 7312 | CD2 | TYR | B | 777 | 24.640 | −10.813 | 104.056 | 1.00 | 68.55 | C |
| ATOM | 7313 | CE1 | TYR | B | 777 | 24.655 | −9.327 | 101.715 | 1.00 | 68.12 | C |
| ATOM | 7314 | CE2 | TYR | B | 777 | 25.850 | −10.404 | 103.498 | 1.00 | 68.30 | C |
| ATOM | 7315 | CZ | TYR | B | 777 | 25.849 | −9.664 | 102.330 | 1.00 | 68.22 | C |
| ATOM | 7316 | OH | TYR | B | 777 | 27.044 | −9.261 | 101.779 | 1.00 | 68.23 | O |
| ATOM | 7317 | N | THR | B | 778 | 20.650 | −13.030 | 105.684 | 1.00 | 69.86 | N |
| ATOM | 7318 | CA | THR | B | 778 | 19.631 | −13.603 | 106.568 | 1.00 | 70.46 | C |
| ATOM | 7319 | C | THR | B | 778 | 19.027 | −12.579 | 107.535 | 1.00 | 70.88 | C |
| ATOM | 7320 | O | THR | B | 778 | 18.070 | −12.881 | 108.246 | 1.00 | 70.93 | O |
| ATOM | 7321 | CB | THR | B | 778 | 20.187 | −14.835 | 107.324 | 1.00 | 70.40 | C |
| ATOM | 7322 | OG1 | THR | B | 778 | 20.727 | −15.759 | 106.374 | 1.00 | 70.46 | O |
| ATOM | 7323 | CG2 | THR | B | 778 | 19.096 | −15.542 | 108.120 | 1.00 | 70.51 | C |
| ATOM | 7324 | N | LEU | B | 779 | 19.590 | −11.373 | 107.547 | 1.00 | 71.48 | N |
| ATOM | 7325 | CA | LEU | B | 779 | 19.028 | −10.227 | 108.278 | 1.00 | 72.14 | C |
| ATOM | 7326 | C | LEU | B | 779 | 18.638 | −10.502 | 109.732 | 1.00 | 72.57 | C |
| ATOM | 7327 | O | LEU | B | 779 | 17.527 | −10.954 | 110.015 | 1.00 | 72.58 | O |
| ATOM | 7328 | CB | LEU | B | 779 | 17.848 | −9.615 | 107.510 | 1.00 | 72.14 | C |
| ATOM | 7329 | CG | LEU | B | 779 | 18.168 | −8.974 | 106.157 | 1.00 | 72.40 | C |
| ATOM | 7330 | CD1 | LEU | B | 779 | 16.893 | −8.644 | 105.418 | 1.00 | 72.76 | C |
| ATOM | 7331 | CD2 | LEU | B | 779 | 19.016 | −7.729 | 106.324 | 1.00 | 72.44 | C |
| ATOM | 7332 | N | SER | B | 780 | 19.553 | −10.194 | 110.647 | 1.00 | 73.16 | N |
| ATOM | 7333 | CA | SER | B | 780 | 19.392 | −10.562 | 112.053 | 1.00 | 73.76 | C |
| ATOM | 7334 | C | SER | B | 780 | 19.389 | −9.364 | 113.002 | 1.00 | 74.13 | C |
| ATOM | 7335 | O | SER | B | 780 | 19.986 | −8.327 | 112.708 | 1.00 | 74.17 | O |
| ATOM | 7336 | CB | SER | B | 780 | 20.484 | −11.555 | 112.456 | 1.00 | 73.75 | C |
| ATOM | 7337 | OG | SER | B | 780 | 20.560 | −12.623 | 111.528 | 1.00 | 73.95 | O |
| ATOM | 7338 | N | PHE | B | 781 | 18.720 | −9.528 | 114.143 | 1.00 | 74.63 | N |
| ATOM | 7339 | CA | PHE | B | 781 | 18.579 | −8.467 | 115.141 | 1.00 | 75.11 | C |
| ATOM | 7340 | C | PHE | B | 781 | 19.447 | −8.691 | 116.380 | 1.00 | 75.47 | C |
| ATOM | 7341 | O | PHE | B | 781 | 19.689 | −9.832 | 116.787 | 1.00 | 75.51 | O |
| ATOM | 7342 | CB | PHE | B | 781 | 17.109 | −8.323 | 115.550 | 1.00 | 75.07 | C |
| ATOM | 7343 | CG | PHE | B | 781 | 16.844 | −7.203 | 116.521 | 1.00 | 75.18 | C |
| ATOM | 7344 | CD1 | PHE | B | 781 | 16.981 | −5.873 | 116.131 | 1.00 | 75.09 | C |
| ATOM | 7345 | CD2 | PHE | B | 781 | 16.441 | −7.480 | 117.823 | 1.00 | 75.12 | C |
| ATOM | 7346 | CE1 | PHE | B | 781 | 16.731 | −4.838 | 117.027 | 1.00 | 74.98 | C |
| ATOM | 7347 | CE2 | PHE | B | 781 | 16.188 | −6.450 | 118.726 | 1.00 | 75.14 | C |
| ATOM | 7348 | CZ | PHE | B | 781 | 16.334 | −5.127 | 118.327 | 1.00 | 75.06 | C |
| ATOM | 7349 | N | ARG | B | 782 | 19.916 | −7.585 | 116.958 | 1.00 | 75.96 | N |
| ATOM | 7350 | CA | ARG | B | 782 | 20.612 | −7.576 | 118.245 | 1.00 | 76.35 | C |
| ATOM | 7351 | C | ARG | B | 782 | 20.209 | −6.333 | 119.043 | 1.00 | 76.87 | C |
| ATOM | 7352 | O | ARG | B | 782 | 20.234 | −5.211 | 118.529 | 1.00 | 76.91 | O |
| ATOM | 7353 | CB | ARG | B | 782 | 22.134 | −7.624 | 118.060 | 1.00 | 76.27 | C |
| ATOM | 7354 | CG | ARG | B | 782 | 22.706 | −9.000 | 117.713 | 1.00 | 76.13 | C |
| ATOM | 7355 | CD | ARG | B | 782 | 24.225 | −8.948 | 117.587 | 1.00 | 76.05 | C |
| ATOM | 7356 | NE | ARG | B | 782 | 24.795 | −10.193 | 117.073 | 1.00 | 75.70 | N |
| ATOM | 7357 | CZ | ARG | B | 782 | 25.310 | −11.164 | 117.824 | 1.00 | 75.36 | C |
| ATOM | 7358 | NH1 | ARG | B | 782 | 25.338 | −11.059 | 119.147 | 1.00 | 74.93 | N |
| ATOM | 7359 | NH2 | ARG | B | 782 | 25.800 | −12.250 | 117.244 | 1.00 | 75.23 | N |
| ATOM | 7360 | N | LYS | B | 783 | 19.821 | −6.553 | 120.296 | 1.00 | 77.48 | N |
| ATOM | 7361 | CA | LYS | B | 783 | 19.429 | −5.489 | 121.216 | 1.00 | 78.13 | C |
| ATOM | 7362 | C | LYS | B | 783 | 20.542 | −5.332 | 122.244 | 1.00 | 78.59 | C |
| ATOM | 7363 | O | LYS | B | 783 | 21.145 | −6.334 | 122.641 | 1.00 | 78.71 | O |
| ATOM | 7364 | CB | LYS | B | 783 | 18.112 | −5.873 | 121.905 | 1.00 | 78.09 | C |
| ATOM | 7365 | CG | LYS | B | 783 | 17.927 | −5.328 | 123.317 | 1.00 | 78.21 | C |
| ATOM | 7366 | CD | LYS | B | 783 | 17.330 | −6.386 | 124.237 | 1.00 | 78.51 | C |
| ATOM | 7367 | CE | LYS | B | 783 | 17.769 | −6.171 | 125.681 | 1.00 | 78.49 | C |
| ATOM | 7368 | NZ | LYS | B | 783 | 17.341 | −7.280 | 126.579 | 1.00 | 78.50 | N |
| ATOM | 7369 | N | PHE | B | 784 | 20.829 | −4.091 | 122.659 | 1.00 | 79.16 | N |
| ATOM | 7370 | CA | PHE | B | 784 | 21.813 | −3.843 | 123.734 | 1.00 | 79.71 | C |
| ATOM | 7371 | C | PHE | B | 784 | 22.373 | −2.420 | 123.876 | 1.00 | 79.88 | C |
| ATOM | 7372 | O | PHE | B | 784 | 22.481 | −1.667 | 122.901 | 1.00 | 79.92 | O |
| ATOM | 7373 | CB | PHE | B | 784 | 22.992 | −4.832 | 123.617 | 1.00 | 79.88 | C |
| ATOM | 7374 | CG | PHE | B | 784 | 23.909 | −4.557 | 122.451 | 1.00 | 80.24 | C |
| ATOM | 7375 | CD1 | PHE | B | 784 | 25.285 | −4.515 | 122.638 | 1.00 | 80.69 | C |
| ATOM | 7376 | CD2 | PHE | B | 784 | 23.402 | −4.330 | 121.170 | 1.00 | 80.52 | C |
| ATOM | 7377 | CE1 | PHE | B | 784 | 26.137 | −4.261 | 121.578 | 1.00 | 80.85 | C |
| ATOM | 7378 | CE2 | PHE | B | 784 | 24.250 | −4.069 | 120.103 | 1.00 | 80.78 | C |
| ATOM | 7379 | CZ | PHE | B | 784 | 25.625 | −4.034 | 120.309 | 1.00 | 80.73 | C |
| ATOM | 7380 | N | ASN | B | 785 | 22.710 | −2.084 | 125.124 | 1.00 | 80.15 | N |
| ATOM | 7381 | CA | ASN | B | 785 | 23.630 | −1.003 | 125.493 | 1.00 | 80.38 | C |
| ATOM | 7382 | C | ASN | B | 785 | 25.039 | −1.620 | 125.449 | 1.00 | 80.52 | C |
| ATOM | 7383 | O | ASN | B | 785 | 25.134 | −2.797 | 125.109 | 1.00 | 80.55 | O |
| ATOM | 7384 | CB | ASN | B | 785 | 23.290 | −0.539 | 126.910 | 1.00 | 80.34 | C |
| ATOM | 7385 | CG | ASN | B | 785 | 23.560 | −1.606 | 127.958 | 1.00 | 80.46 | C |
| ATOM | 7386 | OD1 | ASN | B | 785 | 24.623 | −1.623 | 128.582 | 1.00 | 80.55 | O |
| ATOM | 7387 | ND2 | ASN | B | 785 | 22.603 | −2.511 | 128.145 | 1.00 | 80.30 | N |
| ATOM | 7388 | N | TRP | B | 786 | 26.152 | −0.942 | 125.768 | 1.00 | 80.73 | N |
| ATOM | 7389 | CA | TRP | B | 786 | 26.437 | 0.479 | 126.073 | 1.00 | 80.95 | C |
| ATOM | 7390 | C | TRP | B | 786 | 27.685 | 0.383 | 126.949 | 1.00 | 81.12 | C |
| ATOM | 7391 | O | TRP | B | 786 | 28.591 | 1.215 | 126.860 | 1.00 | 81.16 | O |
| ATOM | 7392 | CB | TRP | B | 786 | 25.349 | 1.265 | 126.812 | 1.00 | 80.97 | C |
| ATOM | 7393 | CG | TRP | B | 786 | 25.895 | 2.541 | 127.417 | 1.00 | 81.10 | C |
| ATOM | 7394 | CD1 | TRP | B | 786 | 26.165 | 2.768 | 128.736 | 1.00 | 81.20 | C |
| ATOM | 7395 | CD2 | TRP | B | 786 | 26.286 | 3.737 | 126.719 | 1.00 | 81.33 | C |
| ATOM | 7396 | NE1 | TRP | B | 786 | 26.679 | 4.033 | 128.907 | 1.00 | 81.36 | N |
| ATOM | 7397 | CE2 | TRP | B | 786 | 26.764 | 4.649 | 127.686 | 1.00 | 81.35 | C |
| ATOM | 7398 | CE3 | TRP | B | 786 | 26.266 | 4.131 | 125.372 | 1.00 | 81.40 | C |
| ATOM | 7399 | CZ2 | TRP | B | 786 | 27.224 | 5.931 | 127.351 | 1.00 | 81.21 | C |
| ATOM | 7400 | CZ3 | TRP | B | 786 | 26.723 | 5.410 | 125.039 | 1.00 | 81.24 | C |
| ATOM | 7401 | CH2 | TRP | B | 786 | 27.192 | 6.292 | 126.028 | 1.00 | 81.17 | C |
| ATOM | 7402 | N | ASP | B | 787 | 27.702 | −0.640 | 127.808 | 1.00 | 81.30 | N |
| ATOM | 7403 | CA | ASP | B | 787 | 28.926 | −1.127 | 128.444 | 1.00 | 81.43 | C |
| ATOM | 7404 | C | ASP | B | 787 | 29.755 | −1.806 | 127.359 | 1.00 | 81.48 | C |
| ATOM | 7405 | O | ASP | B | 787 | 30.983 | −1.862 | 127.432 | 1.00 | 81.48 | O |
| ATOM | 7406 | CB | ASP | B | 787 | 28.606 | −2.131 | 129.558 | 1.00 | 81.43 | C |
| ATOM | 7407 | CG | ASP | B | 787 | 27.716 | −1.546 | 130.647 | 1.00 | 81.48 | C |
| ATOM | 7408 | OD1 | ASP | B | 787 | 27.906 | −0.368 | 131.022 | 1.00 | 81.48 | O |
| ATOM | 7409 | OD2 | ASP | B | 787 | 26.827 | −2.277 | 131.138 | 1.00 | 81.40 | O |
| ATOM | 7410 | N | PHE | B | 788 | 29.048 | −2.326 | 126.357 | 1.00 | 81.57 | N |
| ATOM | 7411 | CA | PHE | B | 788 | 29.632 | −2.813 | 125.114 | 1.00 | 81.65 | C |
| ATOM | 7412 | C | PHE | B | 788 | 30.368 | −1.677 | 124.400 | 1.00 | 81.63 | C |
| ATOM | 7413 | O | PHE | B | 788 | 31.387 | −1.902 | 123.746 | 1.00 | 81.61 | O |
| ATOM | 7414 | CB | PHE | B | 788 | 28.516 | −3.370 | 124.226 | 1.00 | 81.72 | C |
| ATOM | 7415 | CG | PHE | B | 788 | 28.975 | −3.840 | 122.874 | 1.00 | 81.81 | C |
| ATOM | 7416 | CD1 | PHE | B | 788 | 29.337 | −5.170 | 122.672 | 1.00 | 82.00 | C |
| ATOM | 7417 | CD2 | PHE | B | 788 | 29.012 | −2.960 | 121.791 | 1.00 | 81.93 | C |
| ATOM | 7418 | CE1 | PHE | B | 788 | 29.749 | −5.611 | 121.417 | 1.00 | 82.21 | C |
| ATOM | 7419 | CE2 | PHE | B | 788 | 29.419 | −3.391 | 120.535 | 1.00 | 81.98 | C |
| ATOM | 7420 | CZ | PHE | B | 788 | 29.789 | −4.717 | 120.346 | 1.00 | 82.17 | C |
| ATOM | 7421 | N | LEU | B | 789 | 29.839 | −0.463 | 124.539 | 1.00 | 81.65 | N |
| ATOM | 7422 | CA | LEU | B | 789 | 30.429 | 0.731 | 123.935 | 1.00 | 81.66 | C |
| ATOM | 7423 | C | LEU | B | 789 | 31.477 | 1.392 | 124.831 | 1.00 | 81.65 | C |
| ATOM | 7424 | O | LEU | B | 789 | 32.517 | 1.842 | 124.344 | 1.00 | 81.63 | O |
| ATOM | 7425 | CB | LEU | B | 789 | 29.334 | 1.741 | 123.574 | 1.00 | 81.66 | C |
| ATOM | 7426 | CG | LEU | B | 789 | 28.525 | 1.482 | 122.300 | 1.00 | 81.71 | C |
| ATOM | 7427 | CD1 | LEU | B | 789 | 27.137 | 2.094 | 122.404 | 1.00 | 81.70 | C |
| ATOM | 7428 | CD2 | LEU | B | 789 | 29.261 | 2.006 | 121.070 | 1.00 | 81.72 | C |
| ATOM | 7429 | N | SER | B | 790 | 31.194 | 1.449 | 126.132 | 1.00 | 81.65 | N |
| ATOM | 7430 | CA | SER | B | 790 | 32.065 | 2.118 | 127.101 | 1.00 | 81.64 | C |
| ATOM | 7431 | C | SER | B | 790 | 33.285 | 1.271 | 127.465 | 1.00 | 81.63 | C |
| ATOM | 7432 | O | SER | B | 790 | 34.426 | 1.713 | 127.310 | 1.00 | 81.66 | O |
| ATOM | 7433 | CB | SER | B | 790 | 31.286 | 2.483 | 128.374 | 1.00 | 81.67 | C |
| ATOM | 7434 | OG | SER | B | 790 | 30.077 | 3.162 | 128.076 | 1.00 | 81.64 | O |
| ATOM | 7435 | N | LYS | B | 791 | 33.029 | 0.053 | 127.940 | 1.00 | 81.59 | N |
| ATOM | 7436 | CA | LYS | B | 791 | 34.065 | −0.814 | 128.497 | 1.00 | 81.50 | C |
| ATOM | 7437 | C | LYS | B | 791 | 34.594 | −1.792 | 127.452 | 1.00 | 81.41 | C |
| ATOM | 7438 | O | LYS | B | 791 | 35.550 | −1.494 | 126.738 | 1.00 | 81.27 | O |
| ATOM | 7439 | CB | LYS | B | 791 | 33.503 | −1.574 | 129.701 | 1.00 | 81.48 | C |
| ATOM | 7440 | CG | LYS | B | 791 | 34.512 | −1.890 | 130.790 | 1.00 | 81.53 | C |
| ATOM | 7441 | CD | LYS | B | 791 | 33.801 | −2.426 | 132.019 | 1.00 | 81.58 | C |
| ATOM | 7442 | CE | LYS | B | 791 | 34.737 | −2.553 | 133.201 | 1.00 | 81.41 | C |
| ATOM | 7443 | NZ | LYS | B | 791 | 33.990 | −2.994 | 134.409 | 1.00 | 81.35 | N |

Such a search can be carried out commericially, for example, by Schrödinger (Schrödinger LLC, Portland, Oreg., USA) to yield candidate neuraminidase inhibitors that are predicted to bind the neuraminidase active site. Visualization, structural refinement, and docking can be performed using Maestro 7.0, MacroModel 9.0, Prime 1.2, Glide 3.5, and IFD script from Schrödinger, LLC (New York).

### Example 3

### The NanA Neuraminidase of Streptococcus pneumoniae is Involved in Biofilm Formation

Abstract

Streptococcus pneumoniae remains a major cause of bacteremia, pneumonia and otitis media despite vaccines and effective antibiotics. The neuraminidase of S. pneumoniae, which catalyzes the release of terminal sialic acid residues from glycoconjugates, is involved in host colonization in animal models of infection and can provide a new target to prevent pneumococcal infection. We demonstrate that the S. pneumoniae neuraminidase (NanA) cleaves sialic acid and show that it is involved in biofilm formation, indicating an additional role in pathogenesis, and sharing this property with the neuraminidase of Pseudomonas aeruginosa even though we show the two enzymes are phylogenetically divergent. Using an in vitro model of biofilm formation incorporating human airway epithelial cells, we demonstrate that small molecule inhibitors of NanA block biofilm formation and can provide a new target for preventative therapy. This work highlights the role played by the neuraminidase in pathogenesis and represents an important step in drug development to prevent colonization of the respiratory tract by this important pathogen.

### INTRODUCTION

Neuraminidases are widespread among animals and microorganisms, catalyzing the release of terminal sialic acid residues from glycoconjugates (B51). The best characterized is the influenza neuraminidase, required to facilitate spread of the virus. The influenza neuraminidase is not only a key antigen for the highly successful influenza vaccine, it is also the target for the drugs zanamivir and oseltamivir, which have been useful to prevent and ameliorate influenza infection (B57). S. pneumoniae produces at least three distinct neuraminidases (B41); NanA being the most active and most highly expressed at the transcriptional level (B5, B31) that is conserved in all strains (B21, B24, B41). Production of NanA can be detected in vivo, and its expression is upregulated upon interaction with host cells (B27, B39, B46, B59). The pneumococcal neuraminidase modifies host glycoconjugates, including immune defense proteins (B22, B23) and exposes potential binding receptors (B3, B26, B28, B54, B55). Pneumococcal neuraminidase activity also provides a source of carbohydrates for bacterial metabolism, cleaving sugars from the mucosal surface (B8, B23, B61), but whether this significantly contributes to bacterial growth in vivo is not clearly established. Several studies have indicated that nanA mutants colonize the rodent respiratory tract less efficiently than wild type strains (B31, B40, B52) and vaccination with purified NanA affords some protection against nasopharyngeal colonization and otitis media (B29, B30, B53). However, these differences can be mouse strain and animal model dependent (B6, B13, B22, B23).

In addition to targeting host glycoconjugates some bacterial neuraminidases have a role in biofilm formation; presumably by targeting sialylated bacterial exopolysaccharides (B47). S. pneumoniae biofilms have been characterized (B1, B34, B36) and have been observed directly in the middle ear mucosa from children with chronic otitis media (B15), contributing to the colonization process (36). Of note, expression of nanA is upregulated when S. pneumoniae is grown under biofilm conditions (39). There is a need for new therapeutic strategies as serotypes not covered by available vaccines are rising in prevalence by genetic recombination and strain replacement, and are increasingly associated with invasive disease (B7, B20, B45, B58). Without being bound by theory, the neuraminidase of S. pneumoniae is similarly involved in biofilm formation and sought to identify compounds to inhibit its activity in vitro.

Materials and Methods

Bacterial strains and media. S. pneumoniae strains D39 (B4), D39 nanA (B22) and R6 (B 18) and R6 nanA (B23) were grown on trypticase-soy (TS) agar or broth supplemented with 200 U/ml catalase (Worthington) and 1 μg/ml of chloramphenicol for nanA strains. Plate cultures were grown at 37° C. in the presence of carbon dioxide (5%). All chemicals were purchased from Sigma unless otherwise stated.

Epithelial cell culture. Human bronchial epithelial cells, 16HBE14o⁻ and human airway cells, 1HAEo⁻ (Originally from D. Gruenert California Pacific Medical Center Research Institute, San Francisco, Calif., USA), were grown in minimum essential medium with Earle's salts (Cellgro and Gibco respectively) supplemented with 10% fetal bovine serum (Cambrex and Gibco respectively), 100 U/ml penicillin and 100 ug/ml streptomycin. 16HBE14o⁻ cells were additionally supplemented with 2 mM glutamine (Invitrogen). Cells were grown at 37° C. with 5% CO₂ in a humidified incubator.

Neuraminidase assay. NanA was purified as previously described (B19). Neuraminidase activity of NanA was detected using the fluorogenic substrate 2′-(4-methylumbelliferyl)-α-D-N-acetylneuraminic acid (MNN, Sigma). Reactions contained 1.5 mM MNN, 1 nM of NanA in 2.5 mM sodium phosphate buffer (pH 5). Reactions were allowed to incubate for 2 h at 37° C. before fluorescence intensity was measured at excitation and emission wavelengths of 360 nm and 465 nm in a Tecan microplate reader (Männedorf, Switzerland). Compounds were obtained from a variety of sources (Otava, Kiev, Ukraine; Interbioscreen, Moscow, Russia; Chembridge San Diego, USA; Maybridge, Cornwell, UK; Sigma, St Louis, USA; Princeton, Princeton, USA; Lifechem, Burlington, Canada; Enamine, Kiev, Ukraine). Neuraminidase assays with oseltamivir were performed using the hydrolyzed version of the compound. Briefly, a mixture of oseltamivir (300 mg) in methanol (10 ml) was added to 5N NaOH (3 ml). The mixture was stirred overnight and evaporated under reduced pressure. The residue was dissolved in 10 ml H₂O and the washed with 5 ml of ethyl acetate. The aqueous solution was acidified with 5N HCl to pH=2-3. Evaporation of water under reduced pressure gave the hydrolyzed product. The structure of the product was confirmed by NMR and MS. Divalent cations were supplied in the form of calcium, magnesium, ferric and copper chloride. All neuraminidase assays were performed at least in triplicate.

Quantification of asialoGM1 exposure by flow cytometry. 16HBE14o⁻ cells were grown in 24-well plates to confluence and exposed to bacterial supernatants for 3 h followed by three PBS washes. Supernatants were concentrated approximately 30-fold (Amicon Ultra, Millipore) and adjusted for protein quantity. As a control, media alone was also concentrated. Cells were stained with rabbit polyclonal anti aGM1 antibody (Wako) followed by Alexa Fluor 488 donkey anti-rabbit IgG (Molecular Probes). Cells were detached from the plates using 0.02% EGTA in HBSS (Hanks buffered saline solution), fixed with 1% paraformaldehyde and analyzed on a FACSCalibur using CellQuest software (version 3.3; BD). Data were analyzed using WinMDI (version 2.8, Joseph Trotter).

Adherence assay. Adherence assays were performed using 16HBE14o⁻ cells. Bacterial strains were grown to mid-log phase, washed with PBS and 0.7-2×10⁷ cfu of bacterial cells were added to confluent monolayers in 24-well plates (MOI=30). Bacterial cells were allowed to adhere for 1 h at 37° C. before three washes with PBS. Bacteria were dissociated from epithelial cells using TrypLE Express (Gibco) and were serially diluted before plating to determine adherent numbers. The assay was performed with three biological replicates with duplicate technical replicates over two separate experiments.

Biofilm assay. Bacterial strains were grown to mid-log phase before being diluted 1:100 in TS broth and catalase. 100 μl of diluted culture was added in triplicate to 96-well flat bottom tissue culture treated plates (Falcon) and left for 18-24 h at 37° C. with 5% CO₂. Plates were read at 600 nm to determine levels of growth before being washed in water. Adherent biofilm-forming cells were then stained with 125 μL of 1% crystal violet for 15 min before two further washes in water and allowed to dry. Bound crystal violet was then suspended in 200 μL of ethanol, shaken for 15 min and read at 540 nm.

Biofilm formation after epithelial cell interaction. Bacterial strains were grown and inoculated onto 1HAEo⁻ cells as per adherence assay. After the initial PBS washes fresh MEM media was applied before a further 1 h incubation. This removal and addition of fresh media was repeated a further four times before detachment of adherent bacteria using TrypLE Express (Gibco). The detached bacteria were then diluted 1:100 in TS broth and catalase and assayed as per the biofilm assay. Samples were repeated four times, each time in sextuplicate using epithelial cells without bacteria as a negative control. When using inhibitors, inhibitors were present during epithelial cell interaction and in microtitre trays for biofilm formation. Inhibition with NANA was performed using 0.2% w/v NANA (Sigma) (B56). Images of crystal violet stained microtitre wells were taken with a standard digital camera. Fluorescence microscopy was performed using a Zeiss Observer Z1 inverted fluorescence microscope with ApoTome (Zeiss) for optical sectioning and AxioVision software (v 4.6.2.0, Zeiss). Microtitre wells were stained using the live/dead BacLight stain from Invitrogen (Carlsbad, Calif., USA).

Phylogenetic analysis. Our sampling strategy was aimed at maximizing phylogenetic breadth in order to understand the overall pattern of evolution in the neuraminidase/sialidase gene family. We began with a list of well-known neuraminidases including those from V. cholerae, S. typhimurium, C. perfringens, S. pneumoniae, T cruzi, and P. aeruginosa. For each sequence we did a standard BLAST search and collected one sequence from each genus in the list of hits that had an e-value score of 1×10⁻⁵ or lower. Duplicates were deleted. We also included sequences that have been included in other prior publications on the evolution of sialidases (B43). A list of sequences and gene identification numbers is included as supplemental information. Sequences were aligned using the ClustalW algorithm as implemented in the program BioEdit using default settings. Sequences were aligned as amino acids and then transposed back to the original nucleotide sequences maintaining the gaps determined by the initial alignment (5394 characters total, 4124 parsimony informative characters with gaps as a fifth state, 3766 parsimony informative characters with gaps as treated as missing).

A rigorous phylogenetic analysis was undertaken using maximum parsimony (MP) algorithm implemented in PAUP* (B50). 1000 replicates of random addition (RA) was performed followed by the tree branch reconnection (TBR) algorithm using the “multrees” option to save more than one optimal tree if discovered in the search. All characters and state transformations were given equal weight. Terminal gaps were scored as missing data in all analyses. We performed two analyses designating internal gaps as a fifth character state in one and as missing data in the other. Although the trees had many nodes in agreement there were major differences between the structures of the 2 trees. Since gaps can be informative characters (B14, B42) we favor the analysis in which internal gaps are counted as character states. We performed nonparametric bootstrap analyses with 100 iterations consisting of 100 RA replicates followed by TBR to gauge the robustness of the tree.

Statistics. Data significance was determined with a students t-test and for multivariant data with ANOVA followed with a post Dunnetti test using GraphPad Prism software.

Results

Biochemical properties of NanA. To better understand the biochemical properties of NanA and to later facilitiate screening of candidate inhibitory compounds we established an assay to measure its neuraminidase activity. The biochemical activity of NanA was assayed using the fluorogenic sialic acid derivative 2′-(4-methylumbelliferyl)-α-D-N-acetylneuraminic acid (MNN). NanA cleaved the fluorogenic substrate significantly at low nanomolar and even at picomolar concentrations (FIG. 5A). The Km of NanA for this substrate is about 1.4 mM, which is generally comparable to the Km values reported for other neuraminidases. The neuraminidase from Vibrio cholerae requires divalent cations, specifically calcium to be active (B10, B17). We investigated the effect of adding calcium ions to the reaction and observed that calcium was not essential for NanA activity, but did increase activity of NanA by 70% at a concentration of 1 mM (FIG. 5B), a 50% activity increase in the presence of magnesium ions (FIG. 5B) and decreased activity in the presence of iron and copper, presumably due to their larger molecular masses (FIG. 5B). The presence of either copper or ferric ions decreased activity by 90% or greater at millimolar levels.

The ability of sialic acids to competitively inhibit NanA activity was also tested. The two sialic acids used, N-acetylneuraminic acid (NANA) and 2,3-dehydro-2-deoxy-N-acetylneuraminic acid (DANA) were both utilized to obtain co-crystals of NanA (B 19). NANA caused 50% inhibition at 600 μM (FIG. 6). We observed higher inhibition with the transition state analog DANA compared to NANA. DANA reduced activity by 50% at 200 μm.

Phylogeny of the neuraminidases. The neuraminidase superfamily is known to be highly divergent (B43, B51). Without being bound by theory, neuraminidases from organisms that infect a common site can have similar functions and hence can be evolutionarily related; which can be useful in the identification of common inhibitors. To better ascertain the evolutionary relatedness of NanA, a phylogenetic analysis of a number bacterial, eukaryotic and viral neuraminidases was conducted (FIG. 4). NanA clustered closely to the large neuraminidase of C. perfringens (B38) and was more closely related to well characterized bacterial neuraminidases. NanA did not cluster with the neuraminidase from Pseudomonas aeruginosa, which is also involved in respiratory tract colonization.

Biological activity of pneumococcal NanA. Many lung pathogens, including S. pneumoniae and P. aeruginosa, bind to the asialylated ganglioside receptor GM1 (aGM1, Galβ1-3GalNAcβ1-4Galβ1-4Glcβ1-1Cer) (B26). Either purified NanA or concentrated supernatant from wild-type S. pneumoniae strain D39 but not its isogenic nanA mutant exposed aGM1 on the surface of 16HBE14o⁻ epithelial cells (FIG. 7). However, no effect was observed on bacterial adherence nor was there a growth advantage in the presence of airway epithelial cells.

NanA is involved in biofilm formation. As S. pneumoniae nanA expression is upregulated in lung tissue and in biofilm-growing cells (B39) the contribution of nanA to the formation of biofilms was examined (FIG. 12). This increase in expression was exploited by initially growing the bacteria on airway epithelial cells over the course of the day (see Materials and Methods). Adherent bacterial cells were then recovered before setting up the standard microtitre biofilm assay. Exposure of S. pneumoniae to airway epithelial cells prior to setting up the biofilm assay not only caused a significant increase in biofilm formation, but showed the nanA mutant to have significantly reduced capacity to form biofilms. When S. pneumoniae did not have previous airway epithelial cell exposure, no difference in biofilm formation was observed between the WT and nanA strains, nor was much biofilm formation observed. In an S. pneumoniae R6, unencapsulated background, significantly more biofilm was produced and the nanA strain was also significantly reduced in its ability to form biofilms (FIG. 12A). Consistent with NANA acting as an inhibitor in the neuraminidase assay (FIG. 6) adding exogenous NANA to the biofilm assay also resulted in decreased biofilm formation of the wild-type strain (FIG. 12B).

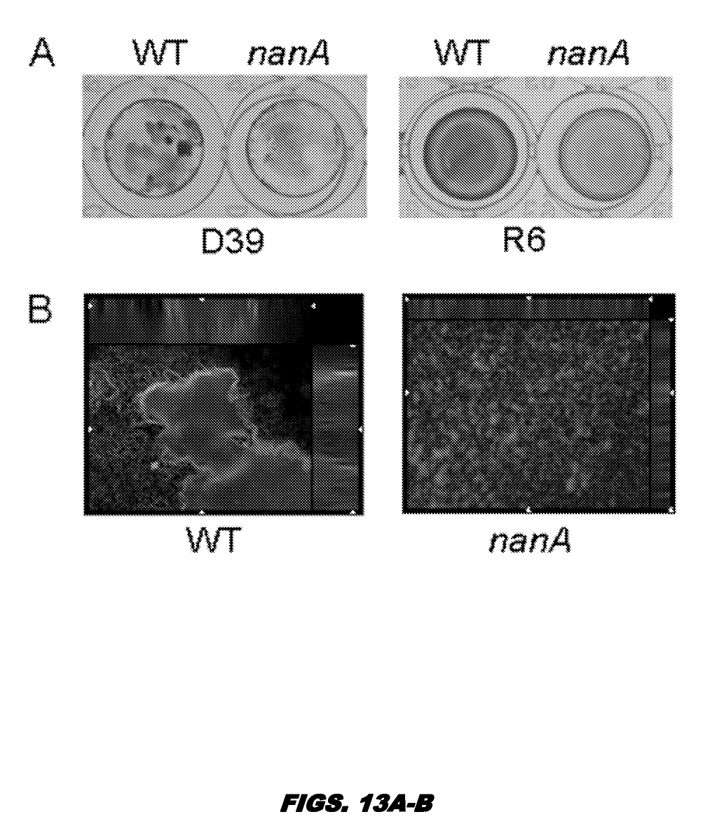

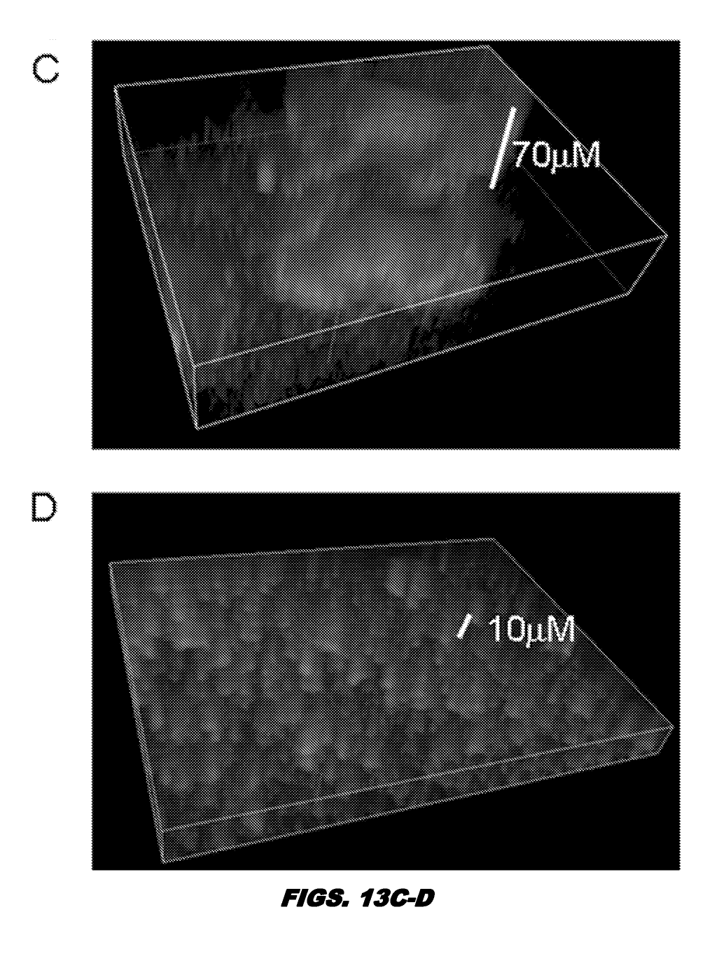

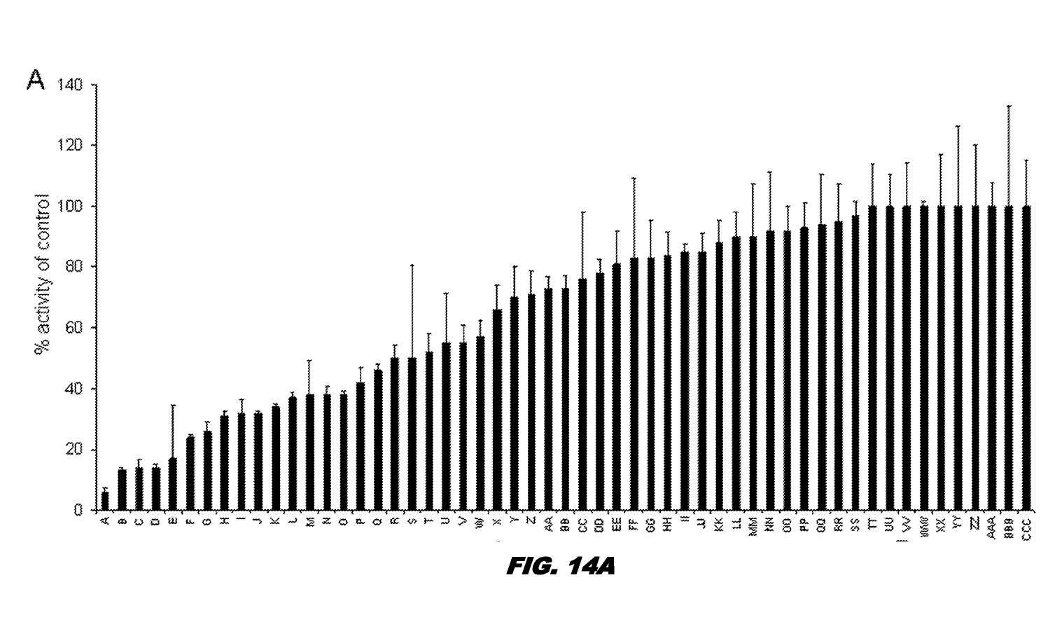

The biofilms produced by R6 were present as even layers of cells on the microtitre plate and the reduction in biofilm observed with the nanA mutant was evident as reduced intensity of crystal violet staining (FIG. 13A). In the D39 background, after exposure to epithelial cells, the WT strain was seen as more intense crystal violet (CV) staining overall compared to the nanA strain (FIG. 13A). Macroscopically, the WT strain had regions of intense CV staining seen as clumps. When the D39 biofilms were examined under the microscope (FIG. 13B) a lattice arrangement of cells was observed in both WT and nanA strains. Regions of concentrated cells were observed with nanA but no macro-structures were observed. However in the WT background structures were observed that had significant height (FIG. 13B-C). These structures were approximately 70 μm in height, as seen on the 3D reconstruction (FIG. 13C). In the nanA background only cells on the initial sections were observed attached to the microtitre well (FIGS. 13B, D).

Identification of small molecule inhibitors to the neuraminidases. A virtual library screen was performed (Schrödinger LLC, Portland, Oreg., USA) (B25, B44) that identified small molecules that were predicted to interact with the active site of the enzyme. As a control we tested the ability of the influenza neuraminidase inhibitor oseltamivir to inhibit NanA (FIG. 10). Oseltamivir had an IC₅₀ of 2 mM. A number of compounds identified from this screen showed high degrees of inhibition in vitro (FIG. 14A). A lead compound, designated XX1 (FIG. 14D), with a pyrrolidine-2,3-dione chemical scaffold was found to inhibit NanA over a range of concentrations and in a dose-dependent manner (FIG. 14B). An IC₅₀ of 28 μM was determined. The inhibition afforded by XX1 to NanA was greater than 7, 20 and 70 times more effective than DANA, NANA and oseltamivir, respectively. At a concentration of 100 μM we observed a significant reduction in biofilm formation with XX1 and at 400 μM the levels of biofilm formation were comparative to the nanA strain (FIG. 14C). XX1 did not affect growth of planktonic cells.

Discussion

For S. pneumoniae, the biofilm phenotype is most evident in vivo (B 15), or as we show, under in vitro conditions that favor neuraminidase expression. We were able to demonstrate a difference in biofilm production after growth on human airway cells, which has been shown to induce nanA expression (B27, B39, B46) and reduce capsule production (B16). By selecting for organisms on human airway cells, we apparently mimic a in vivo process in which encapsulated organisms that are able to avoid mucus entrapment and clearance gradually produce less capsule to facilitate epithelial attachment (B20). This association between capsule expression and biofilm formation was evident with the R6 strain. R6 does not produce capsule and we observed it to produce significant biofilms, with its nanA mutant displaying reduced propensity for biofilm formation. This reduced capsule is important in initial airway colonization both from facilitating initial adherence and promoting cell-to-cell interaction for biofilm formation (B20). This is also consistent with other studies that showed cells induced to a biofilm state had greater propensity to cause pneumonia compared to planktonic cells (B39).

Without being bound by theory, the role of the neuraminidase lies somewhere between the two processes of cluster formation and biofilm maturation (B1). This is based on the fact that the biofilms observed with the wild-type strain resembled those seen in mature biofilms grown under flow conditions (B2) and while the nanA strain still had regions of clustered cells it had no large cellular structures. It should be noted that some differences are also to be expected when comparing static and continuous flow systems. Even though the development and composition of biofilms is complex and numerous genes and proteins are differentially expressed (B1, B34, B39), the high expression of nanA in biofilms and subsequent data presented here, indicate a crucial role for this enzyme in biofilm production. A recent study by Trappetti et al (B56) demonstrated the role of sialic acid in pneumococcal biofilm formation. Addition of sialic acid and not other sugars resulted in increased biofilm formation as well as increased organisms present in the nasopharynx in a murine model of colonization (B56). NANA at high concentrations inhibits biofilm formation by S. pneumoniae by occupying the binding site of NanA. Based on this study and our own work, the liberation of sialic acid residues from the airway epithelium by the action of NanA appears to contribute to biofilm formation by S. pneumoniae, and hence would be a desirable target for therapeutic intervention.

Even though nanA is the most highly expressed neuraminidase gene the other neuraminidases of S. pneumoniae can contribute to biofilm formation. In the serotype 4 background (TIGR4) nanB is involved to a small extent in biofilm formation (B36). It should be noted that a mutation exists in nanA in this strain affecting its cell wall attachment. These studies highlighted the important role biofilms play in pathogenesis, establishing a link between biofilm formation and colonization of the murine nasopharynx. Our data are consistent with these observations indicating a reduced capacity to form biofilms is behind the reduced pathogenesis of the neuraminidase (nanA) mutant in animal models (B31, B40). Based on this work and others a model for the establishment of infection in the respiratory tract by S. pneumoniae can be established. Encapsulated cells reach the mucus layer where the capsule is required to avoid mucous entrapment. NanA is also able to utilize the mucin as a carbon source (B61). Upon reaching the respiratory epithelium capsule expression is down regulated to facilitate intimate attachment. It is at this point that neuraminidase expression is increased and cells begin to establish a biofilm state.

Another respiratory pathogen, P. aeruginosa also produces a neuraminidase (NanPs), important for biofilm formation and pathogenesis in animal models (B47), indicating a conserved role in pathogenesis for the two neuraminidases. Desialylated glyco-lipids provide receptors for many of the common bacterial pulmonary pathogens including both S. pneumoniae and P. aeruginosa (B26), which bind to the exposed GalNAcβ1-4Gal residues when terminal sialic acid is cleaved. We demonstrated that NanA like P. aeruginosa (B47) was capable of exposing this receptor on human airway cells. The neuraminidase activity associated with intact organisms, either S. pneumoniae or P. aeruginosa (B47) was not associated with increased bacterial attachment, which is an interesting observation given that the desialylation of airway mucosal cells by the influenza neuraminidase increases susceptibility to secondary infection often caused by S. pneumoniae (B33).

The crystal structures of both NanA and NanPs were recently solved (B 19, B60). Structural analysis indicated that while both enzymes had similar overall structure their active sites were remarkably different, indicating differences in substrate specificity and biochemical function (B9, B47). We also observed that both enzymes are phylogenetically distinct, with NanA clustering closer to other well characterized canonical neuraminidases and NanPs located in a phylogenetically diverse branch of the tree. Yet despite their differences in structure, and different substrates, both neuraminidases possess similar functions in pathogenesis.

The neuraminidase from Vibrio cholerae requires divalent cations, specifically calcium, to be active (B10, B17), while we observed an increase in activity in the presence of calcium ions it was not an absolute requirement for activity. This is an analogous observation with the neuraminidase of P. aeruginosa (B9). The inhibition we did observe with iron and copper ions is consistent with metal ions inhibiting such enzymes (B17). The increase of activity with calcium can indicat the presence of a calcium binding site as in V. cholerae (B10) and C. perfringens (B38) although this is not a categorical feature of neuraminidases nor was one identified from the crystal structure (B9, B11, B19, B48, B60). However, the presence of a calcium binding site would not be incongruous given the close phylogenetic relationship between NanA and the other well characterized bacterial neuraminidases.

New therapeutic strategies needed against S. pneumoniae as serotypes not covered by available vaccines are rising in prevalence by genetic recombination and are increasingly associated with invasive disease (B7, B20, B45, B58); in addition, antibiotic resistance amongst S. pneumoniae is a growing problem (B32). We identified a number of compounds based on in silico docking studies that were significantly more inhibitory than the sialic acids NANA and DANA, and the influenza inhibitor oseltamivir. Many neuraminidases have been crystallized in the presence of the sialic acid DANA (B11, B19, B35, B38) and we subsequently observed it to inhibit the neuraminidase activity of NanA better than NANA. Although the inhibition observed by DANA was not as great as some neuraminidases tested to-date, it is within the observed ranges of inhibition (B12, B48). Our lead compound XX1 was found to inhibit the neuraminidase activity of NanA in the low micromolar range and also inhibit the organisms ability to form biofilms. Some work has been done looking into inhibition of other bacterial neuraminidases but the most work to date has been with the trypanosome trans-sialidases (B37, B48, B49). Studies to-date have identified trypanosome inhibitors with IC₅₀'s in the range of 100-300 μM, indicating that the studies presented here are already progressing well comparative to the scientific community. We are actively involved in synthesizing XX1 in an attempt to examine its ability to prevent infection in vivo.

Based on structural data, inhibitors developed to the pneumococcal neuraminidase will be organism specific. The NanPs neuraminidase of P. aeruginosa has a significantly different active site. The binding pocket of NanA is tight while NanPs has an open conformation that can require different inhibitor structures for effective inhibition (B19). Thus, neuraminidase inhibitor delivery to the lung can be used as a prophylaxis to circumvent bacterial pneumonia post influenza infection and also to at-risk populations.

Despite biochemical, structural and phylogenetic differences we demonstrate a common role for the neuraminidases of S. pneumoniae and P. aeruginosa in biofilm formation and the pathogenesis of respiratory tract infection. Due to the importance of biofilms in colonization of S. pneumoniae we have begun to identify inhibitors targeting the pneumococcal neuraminidase to prevent infection. This study represents a starting point in the development of a new drug against pneumococcal pneumonia.

Supporting Information

Accession numbers. The GenBank (http://www.ncbi.nlm.nih.gov/Entrez/) accession numbers utilized for phylogenetic analysis are: Verrucomicrobium spinosum gi|164421336:353068-354225, Blastopirellula marina gi|87311313:89394-90503, Lentisphaera araneosa gi|149198907:89577-90743, Propionibacterium acnes gi|50841496:752060-754375, Ruminococcus lactaris gi|197302028:32228-35857, Erysipelothrix rhusiopathiae gi|13516389:295-3807, Pasteurella multocida gi|15601865:1176085-1179327, Actinomyces odontolyticus gi|145845834:308755-310992, Mannheimia haemolytica gi|125433996:1-2376, Haemophilus parasuis gi|167854877:54475-56886, Bacteroides fragilis gi|53711291:4836372-4838006, Akkermansia muciniphila gi|187734516:2229943-2231967, Capnocytophaga canimorsus gi|194454827:2197-3765, Parabacteroides distasonis gi|150006674:3525685-3527310, Shewanella pealeana gi|157959830:1838982-1841831, Flavobacteriales bacterium gi|88710837:680637-681797, Rhodopirellula baltica gi|32470666:1724290-1725519, Opitutaceae bacterium gi|153892517:3249-4847, Sassharopolyspora erythraea gi|134096620:5769332-5771182, Pseudoalteromonas haloplanktis gi|77361923:196316-197458, Chthoniobacter flavus gi|196231426:66519-67730, Janibacter sp. gi|84494251:767782-770736, Monosiga brevicollis gi|167534964:1-984, Strongylocentrotus purpuratus gi/115616575:1-719, Planctomyces maris gi|149177549:10030-11205, Acinetobacter baumannii gi|169632029:647539-649371, Opitutaceae bacterium gi|153890920:5481-6641, Danio rerio gi|148539964:69-1220, Corynebacterium diptheriae gi|38232642:512872-515037, Gemmata obscuriglobus gi|163804184:63331-64515, Streptomyces coelicolor gi|32141095:7255596-7257542, Takifugu rubripes gi|148372013:1-87, V. cholerae gi|12057212:1933231-1935654, C. perfringens nanI gi|18308982:900997-903081, C. perfringens nanH gi|18308982:904693-905499, P. aeruginosa gi|110227054:3150886-3152202, C. septicum gi|40662, C. sordellii gi|1710442, A. viscosus gi|39254, Trypanosoma rangeli gi|2894809, T. cruzi gi|162265, T. cruzi SAPA (shed-acute-phase-antigen) gi|10943, Micromonosporta viridifaciens gi|216782:816-2759, Arthrobacter ureafaciens gi|60544840, Influenzae A H5N1 gi|108671038, Macrobdella decora gi|1353880, Salmonella typhimurium gi|16763390:1002088-1003326, S. pneumoniae gi/116515308:1522475-1525468, Arcanobacterium pyogenes gi|18146340:1026-6239, Xenopus laevis gi|148228846:180-1376, Trichomonas vaginalis gi|123473002:1-1050, Rattus norvegicus gi|71896601:59-1288, Bos taurus gi|149676185:61-219, 650-842, 1541-1803, 2490-2672, 2849-3071, 3185-3411, and Monodelphis domestica gi|126309689:1-1404.

### REFERENCES FOR EXAMPLE 3

B1. Allegrucci, M., F. Z. Hu, K. Shen, J. Hayes, G. D. Ehrlich, J. C. Post, and K. Sauer. 2006. Phenotypic characterization of Streptococcus pneumoniae biofilm development. J Bacteriol 188:2325-2335.
B2. Allegrucci, M., and K. Sauer. 2007. Characterization of colony morphology variants isolated from Streptococcus pneumoniae biofilms. J Bacteriol 189:2030-2038.
B3. Andersson, B., J. Dahmen, T. Frejd, H. Leffler, G. Magnusson, G. Noori, and C. S. Eden. 1983. Identification of an active disaccharide unit of a glycoconjugate receptor for pneumococci attaching to human pharyngeal epithelial cells. J Exp Med 158:559-570.
B4. Avery, 0. T., C. M. MacLeod, and M. McCarty. 1944. Studies on the chemical nature of the substance inducing transformation of pneumococcal types: inductions of transformation by a desoxyribonucleic acid fraction isolated from pneumococcus type III. Journal of Experimental Medicine 79:137-158.
B5. Berry, A. M., R. A. Lock, and J. C. Paton. 1996. Cloning and characterization of nanB, a second Streptococcus pneumoniae neuraminidase gene, and purification of the NanB enzyme from recombinant Escherichia coli. J Bacteriol 178:4854-4860.
B6. Berry, A. M., and J. C. Paton. 2000. Additive attenuation of virulence of Streptococcus pneumoniae by mutation of the genes encoding pneumolysin and other putative pneumococcal virulence proteins. Infect Immun 68:133-140.
B7. Brueggemann, A. B., R. Pai, D. W. Crook, and B. Beall. 2007. Vaccine escape recombinants emerge after pneumococcal vaccination in the United States. PLoS Pathog 3:e168.
B8. Burnaugh, A. M., L. J. Frantz, and S. J. King. 2008. Growth of Streptococcus pneumoniae on human glycoconjugates is dependent upon the sequential activity of bacterial exoglycosidases. J Bacteriol 190:221-230.
B9. Cacalano, G., M. Kays, L. Saiman, and A. Prince. 1992. Production of the Pseudomonas aeruginosa neuraminidase is increased under hyperosmolar conditions and is regulated by genes involved in alginate expression. J Clin Invest 89:1866-1874.
B10. Crennell, S., E. Garman, G. Layer, E. Vimr, and G. Taylor. 1994. Crystal structure of Vibrio cholerae neuraminidase reveals dual lectin-like domains in addition to the catalytic domain. Structure 2:535-544.
B11. Crennell, S. J., E. F. Garman, W. G. Layer, E. R. Vimr, and G. L. Taylor. 1993. Crystal structure of a bacterial sialidase (from Salmonella typhimurium LT2) shows the same fold as an influenza virus neuraminidase. Proc Natl Acad Sci USA 90:9852-9856.
B12. Crennell, S. J., E. F. Garman, C. Philippon, A. Vasella, W. G. Layer, E. R. Vimr, and G. L. Taylor. 1996. The structures of Salmonella typhimurium LT2 neuraminidase and its complexes with three inhibitors at high resolution. J. Mol. Biol. 259:264-280.
B13. Gingles, N. A., J. E. Alexander, A. Kadioglu, P. W. Andrew, A. Kerr, T. J. Mitchell, E. Hopes, P. Denny, S. Brown, H. B. Jones, S. Little, G. C. Booth, and W. L. McPheat. 2001. Role of genetic resistance in invasive pneumococcal infection: identification and study of susceptibility and resistance in inbred mouse strains. Infect Immun 69:426-434.
B14. Giribet, G., and W. C. Wheeler. 1999. On gaps. Mol Phylogenet Evol 13:132-143.
B15. Hall-Stoodley, L., F. Z. Hu, A. Gieseke, L. Nistico, D. Nguyen, J. Hayes, M. Forbes, D. P. Greenberg, B. Dice, A. Burrows, P. A. Wackym, P. Stoodley, J. C. Post, G. D. Ehrlich, and J. E. Kerschner. 2006. Direct detection of bacterial biofilms on the middle-ear mucosa of children with chronic otitis media. Jama 296:202-211.
B16. Hammerschmidt, S., S. Wolff, A. Hocke, S. Rosseau, E. Muller, and M. Rohde. 2005. Illustration of pneumococcal polysaccharide capsule during adherence and invasion of epithelial cells. Infect Immun 73:4653-4667.
B17. Holmquist, L. 1975. Activation of Vibrio cholerae neuraminidase by divalent cations. FEBS Lett 50:269-271.
B18. Hoskins, J., W. E. Alborn, Jr., J. Arnold, L. C. Blaszczak, S. Burgett, B. S. DeHoff, S. T. Estrem, L. Fritz, D. J. Fu, W. Fuller, C. Geringer, R. Gilmour, J. S. Glass, H. Khoja, A. R. Kraft, R. E. Lagace, D. J. LeBlanc, L. N. Lee, E. J. Lefkowitz, J. Lu, P. Matsushima, S. M. McAhren, M. McHenney, K. McLeaster, C. W. Mundy, T. I. Nicas, F. H. Norris, M. O'Gara, R. B. Peery, G. T. Robertson, P. Rockey, P. M. Sun, M. E. Winkler, Y. Yang, M. Young-Bellido, G. Zhao, C. A. Zook, R. H. Baltz, S. R. Jaskunas, P. R. Rosteck, Jr., P. L. Skatrud, and J. I. Glass. 2001. Genome of the bacterium Streptococcus pneumoniae strain R6. J Bacteriol 183:5709-5717.
B19. Hsiao, Y. S., D. Parker, A. J. Ratner, A. Prince, and L. Tong. 2009. Crystal structures of respiratory pathogen neuraminidases. Biochemical and Biophysical Research Communications:Accepted Jan. 15, 2009.
B20. Kadioglu, A., J. N. Weiser, J. C. Paton, and P. W. Andrew. 2008. The role of Streptococcus pneumoniae virulence factors in host respiratory colonization and disease. Nat Rev Microbiol 6:288-301.
B21. Kelly, R. T., S. Farmer, and D. Greiff. 1967. Neuraminidase activities of clinical isolates of Diplococcus pneumoniae. J Bacteriol 94:272-273.
B22. King, S. J., K. R. Hippe, J. M. Gould, D. Bae, S. Peterson, R. T. Cline, C. Fasching, E. N. Janoff, and J. N. Weiser. 2004. Phase variable desialylation of host proteins that bind to Streptococcus pneumoniae in vivo and protect the airway. Mol Microbiol 54:159-171.
B23. King, S. J., K. R. Hippe, and J. N. Weiser. 2006. Deglycosylation of human glycoconjugates by the sequential activities of exoglycosidases expressed by Streptococcus pneumoniae. Mol Microbiol 59:961-974.
B24. King, S. J., A. M. Whatmore, and C. G. Dowson. 2005. NanA, a neuraminidase from Streptococcus pneumoniae, shows high levels of sequence diversity, at least in part through recombination with Streptococcus oralis. J Bacteriol 187:5376-5386.
B25. Kontoyianni, M., L. M. McClellan, and G. S. Sokol. 2004. Evaluation of docking performance: comparative data on docking algorithms. J Med Chem 47:558-565.
B26. Krivan, H. C., D. D. Roberts, and V. Ginsburg. 1988. Many pulmonary pathogenic bacteria bind specifically to the carbohydrate sequence GalNAc beta 1-4Gal found in some glycolipids. Proc Natl Acad Sci USA 85:6157-6161.
B27. LeMessurier, K. S., A. D. Ogunniyi, and J. C. Paton. 2006. Differential expression of key pneumococcal virulence genes in vivo. Microbiology 152:305-311.
B28. Linder, T. E., R. L. Daniels, D. J. Lim, and T. F. DeMaria. 1994. Effect of intranasal inoculation of Streptococcus pneumoniae on the structure of the surface carbohydrates of the chinchilla eustachian tube and middle ear mucosa. Microb Pathog 16:435-441.
B29. Lock, R. A., J. C. Paton, and D. Hansman. 1988. Comparative efficacy of pneumococcal neuraminidase and pneumolysin as immunogens protective against Streptococcus pneumoniae. Microb Pathog 5:461-467.
B30. Long, J. P., H. H. Tong, and T. F. DeMaria. 2004. Immunization with native or recombinant Streptococcus pneumoniae neuraminidase affords protection in the chinchilla otitis media model. Infect Immun 72:4309-4313.
B31. Manco, S., F. Hernon, H. Yesilkaya, J. C. Paton, P. W. Andrew, and A. Kadioglu. 2006. Pneumococcal neuraminidases A and B both have essential roles during infection of the respiratory tract and sepsis. Infect Immun 74:4014-4020.
B32. McCormick, A. W., C. G. Whitney, M. M. Farley, R. Lynfield, L. H. Harrison, N. M. Bennett, W. Schaffner, A. Reingold, J. Hadler, P. Cieslak, M. H. Samore, and M. Lipsitch. 2003. Geographic diversity and temporal trends of antimicrobial resistance in Streptococcus pneumoniae in the United States. Nat. Med 9:424-430.
B33. McCullers, J. A. 2006. Insights into the interaction between influenza virus and pneumococcus. Clin Microbiol Rev 19:571-582.
B34. Moscoso, M., E. Garcia, and R. Lopez. 2006. Biofilm formation by Streptococcus pneumoniae: role of choline, extracellular DNA, and capsular polysaccharide in microbial accretion. J Bacteriol 188:7785-7795.
B35. Moustafa, I., H. Connaris, M. Taylor, V. Zaitsev, J. C. Wilson, M. J. Kiefel, M. von Itzstein, and G. Taylor. 2004. Sialic acid recognition by Vibrio cholerae neuraminidase. J Biol Chem 279:40819-40826.
B36. Munoz-Elias, E. J., J. Marcano, and A. Camilli. 2008. Isolation of Streptococcus pneumoniae biofilm mutants and their characterization during nasopharyngeal colonization. Infect Immun 76:5049-5061.
B37. Neres, J., M. L. Brewer, L. Ratier, H. Botti, A. Buschiazzo, P. N. Edwards, P. N. Mortenson, M. H. Charlton, P. M. Alzari, A. C. Frasch, R. A. Bryce, and K. T. Douglas. 2009. Discovery of novel inhibitors of Trypanosoma cruzi trans-sialidase from in silico screening. Bioorg Med Chem Lett 19:589-596.
B38. Newstead, S. L., J. A. Potter, J. C. Wilson, G. Xu, C. H. Chien, A. G. Watts, S. G. Withers, and G. L. Taylor. 2008. The structure of Clostridium perfringens NanI sialidase and its catalytic intermediates. J Biol Chem 283:9080-9088.
B39. Oggioni, M. R., C. Trappetti, A. Kadioglu, M. Cassone, F. Iannelli, S. Ricci, P. W. Andrew, and G. Pozzi. 2006. Switch from planktonic to sessile life: a major event in pneumococcal pathogenesis. Mol Microbiol 61:1196-1210.
B40. Orihuela, C. J., G. Gao, K. P. Francis, J. Yu, and E. I. Tuomanen. 2004. Tissue-specific contributions of pneumococcal virulence factors to pathogenesis. J Infect Dis 190:1661-1669.
B41. Pettigrew, M. M., K. P. Fennie, M. P. York, J. Daniels, and F. Ghaffar. 2006. Variation in the presence of neuraminidase genes among Streptococcus pneumoniae isolates with identical sequence types. Infect Immun 74:3360-3365.
B42. Phillips, A., D. Janies, and W. Wheeler. 2000. Multiple sequence alignment in phylogenetic analysis. Mol Phylogenet Evol 16:317-330.
B43. Roggentin, P., R. Schauer, L. L. Hoyer, and E. R. Vimr. 1993. The sialidase superfamily and its spread by horizontal gene transfer. Mol Microbiol 9:915-921.
B44. Sherman, W., T. Day, M. P. Jacobson, R. A. Friesner, and R. Farid. 2006. Novel procedure for modeling ligand/receptor induced fit effects. J Med Chem 49:534-553.
B45. Singleton, R. J., T. W. Hennessy, L. R. Bulkow, L. L. Hammitt, T. Zulz, D. A. Hurlburt, J. C. Butler, K. Rudolph, and A. Parkinson. 2007. Invasive pneumococcal disease caused by nonvaccine serotypes among alaska native children with high levels of 7-valent pneumococcal conjugate vaccine coverage. Jama 297:1784-1792.
B46. Song, X. M., W. Connor, K. Hokamp, L. A. Babiuk, and A. A. Potter. 2008. Streptococcus pneumoniae early response genes to human lung epithelial cells. BMC Res Notes 1:64.
B47. Soong, G., A. Muir, M. I. Gomez, J. Waks, B. Reddy, P. Planet, P. K. Singh, Y. Kaneko, M. C. Wolfgang, Y. S. Hsiao, L. Tong, and A. Prince. 2006. Bacterial neuraminidase facilitates mucosal infection by participating in biofilm production. J Clin Invest 116:2297-2305.
B48. Streicher, H.2004. Inhibition of Microbial Sialidases—What has Happened Beyond the Influenza Virus? Curr Med Chem 3:149-161.
B49. Streicher, H., and H. Busse. 2006. Building a successful structural motif into sialylmimetics-cyclohexenephosphonate monoesters as pseudo-sialosides with promising inhibitory properties. Bioorg Med Chem 14:1047-1057.
B50. Swofford, D. L. 1998. PAUP*: Phylogenetic analysis using parsimony (*and other methods). 4.0 beta ed. Sinauer, Sunderland, Massachussetts.
B51. Taylor, G. 1996. Sialidases: structures, biological significance and therapeutic potential. Curr Opin Struct Biol 6:830-837.
B52. Tong, H. H., L. E. Blue, M. A. James, and T. F. DeMaria. 2000. Evaluation of the virulence of a Streptococcus pneumoniae neuraminidase-deficient mutant in nasopharyngeal colonization and development of otitis media in the chinchilla model. Infect Immun 68:921-924.
B53. Tong, H. H., D. Li, S. Chen, J. P. Long, and T. F. DeMaria. 2005. Immunization with recombinant Streptococcus pneumoniae neuraminidase NanA protects chinchillas against nasopharyngeal colonization. Infect Immun 73:7775-7778.
B54. Tong, H. H., X. Liu, Y. Chen, M. James, and T. Demaria. 2002. Effect of neuraminidase on receptor-mediated adherence of Streptococcus pneumoniae to chinchilla tracheal epithelium. Acta Otolaryngol 122:413-419.
B55. Tong, H. H., M. A. McIver, L. M. Fisher, and T. F. DeMaria. 1999. Effect of lacto-N-neotetraose, asialoganglioside-GM1 and neuraminidase on adherence of otitis media-associated serotypes of Streptococcus pneumoniae to chinchilla tracheal epithelium. Microb Pathog 26:111-119.
B56. Trappetti, C., A. Kadioglu, C. Carter, J. Hayre, F. Iannelli, G. Pozzi, P. W. Andrew, and M. R. Oggioni. 2009. Sialic Acid: A Preventable Signal for Pneumococcal Biofilm Formation, Colonization, and Invasion of the Host. J Infect Dis 199:1497-1505.
B57. von Itzstein, M. 2007. The war against influenza: discovery and development of sialidase inhibitors. Nat Rev Drug Discov 6:967-974.
B58. World Health Organisation (WHO). 2007. Pneumococcal conjugate vaccine for childhood immunization—WHO position paper. Weekly epidemiological record. World Health Organisation.
B59. Williamson, Y. M., R. Gowrisankar, D. L. Longo, R. Facklam, I. K. Gipson, E. P. Ades, G. M. Carlone, and J. S. Sampson. 2008. Adherence of nontypeable Streptococcus pneumoniae to human conjunctival epithelial cells. Microb Pathog 44:175-185.
B60. Xu, G., X. Li, P. W. Andrew, and G. L. Taylor. 2008. Structure of the catalytic domain of Streptococcus pneumoniae sialidase NanA. Acta Cryst. F64:772-775.
B61. Yesilkaya, H., S. Manco, A. Kadioglu, V. S. Terra, and P. W. Andrew. 2008. The ability to utilize mucin affects the regulation of virulence gene expression in Streptococcus pneumoniae. FEMS Microbiol Lett 278:231-235.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method for reducing or inhibiting bacterial biofilm formation, the method comprising contacting a surface with a bacterial neuraminidase inhibitor for a sufficient time so as to modulate bacterial neuraminidase activity, thereby reducing or inhibiting formation of the biofilm.

2. The method of claim 1, wherein the surface comprises a biofilm.

3. The method of claim 1, wherein the biofilm is produced by a bacterium.

4. The method of claim 1, wherein the neuraminidase inhibitor is a compound comprising Formula (I): 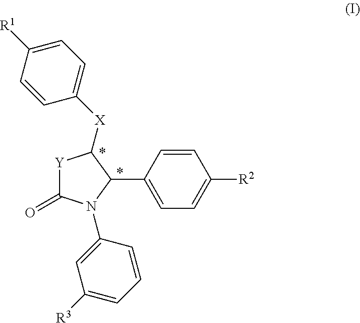
wherein,
R¹ is H, halogen, cyano, azido, nitro, C₁-C₆ alkyl, or C₁-C₆ alkoxy;
R² is H, halogen, cyano, azido, nitro, C₁-C₆ alkyl, or C₁-C₆ alkoxy;
R³ is H, —CO₂R⁴ or —CON(R⁴)₂;
each R⁴ is, independently, H or C₁-C₆ alkyl;
X is —CH₂—, —(C═O)—, —(C═NH)—, —(C═N—O—C₁-C₆-alkyl)-, or —(C═S)—; and
Y is —(C═O)—, —(C═NH)—, —(C═N—O—C₁-C₆-alkyl)-, or —(C═S)—,
or a pharmaceutically acceptable salt or hydrate thereof.

5. The method of claim 1, wherein the neuraminidase inhibitor is a compound comprising Formula (A),

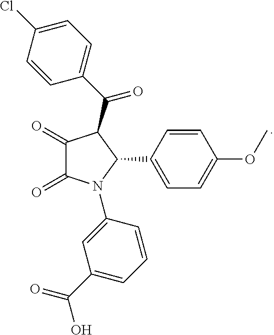

6. The method of claim 1, wherein the neuraminidase inhibitor is a compound comprising Formula (X),

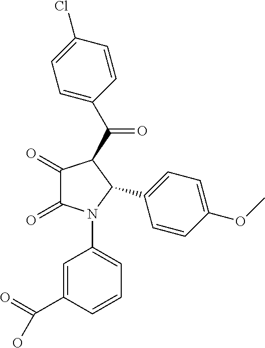

7. The method of claim 1, wherein the neuraminidase inhibitor comprises oseltamivir, peramivir, zanamivir, or a variant thereof.

8. The method of claim 1, wherein the neuraminidase inhibitor is an antibody that specifically binds to the NanA protein of S. pneumoniae or a fragment thereof; an antisense RNA or antisense DNA that inhibits expression of a NanA polypeptide; a siRNA that specifically targets a NanA gene, a peptide comprising at least 10 amino acids of SEQ ID NO: 2 wherein the peptide competes with endogenous NanA for ligand binding; or a combination thereof.

9. The method of claim 3, wherein the bacterium is a Gram-negative or Gram-positive bacterium.

10. The method of claim 3, wherein the bacterium is Pseudomonas, Streptococcus, Haemophilus, Vibrio, Streptococcus pneumoniae, Haemophilus influenzae, or Vibrio cholerae.

11. The method of claim 1, wherein the surface comprises a cellular surface of a subject, an oral surface of a subject, or an in vitro surface.

12. The method of claim 1, wherein the contacting comprises administering the neuraminidase inhibitor to a subject via subcutaneous, intra-muscular, intra-peritoneal, or intravenous injection; infusion; oral, nasal, or topical delivery; or a combination thereof.

13. The method of claim 11 or 12, wherein the subject is a human, mouse, rat, bird, dog, cat, cow, horse, or pig.

14. The method of claim 1, wherein the surface comprises a prosthetic graft, a catheter, a wound dressing, a wound site, a medical device, a contact lens, an implanted device, an oral device, a pipe, or industrial equipment.

15. The method of claim 1, wherein the contacting comprises applying the neuraminidase inhibitor to a surface of a prosthetic graft, a catheter, a wound dressing, a wound site, or a medical device, and further comprises administering the neuraminidase inhibitor to the subject prior to or during or after the implantation of the prosthetic graft, the implantation of the catheter, the application to the wound site, the application of the wound dressing, or the implantation or insertion of the medical device.

16. The method of claim 14, wherein industrial equipment is found in a GMP facility.

17. The method of claim 14, wherein industrial equipment comprises a plumbing system.

18. The method of claim 1, further comprising administering an effective amount of a therapeutic composition to the subject, the therapeutic composition being different than the neuraminidase inhibitor.

19. The method of claim 18, wherein administering occurs sequentially or simultaneously.

20. The method of claim 18, wherein the therapeutic composition comprises an antibiotic.

21. The method of claim 20, wherein the antibiotic comprises a cephalosporin, a macrolide, a penicillin, a quinolone, a sulfonamide, a tetracycline, or any combination thereof.

22. The method of claim 1, wherein the neuraminidase inhibitor is in a formulation of a paste, a liquid, a powder, a gel, or a tablet.

23. The method of claim 22, wherein the paste formulation further comprises an abrasive.

24. The method of claim 23, wherein the paste formulation is toothpaste.

25. The method of claim 22, wherein the liquid formulation is a mouthwash.

26. A method for treating a biofilm production-related disorder in a subject in need thereof, the method comprising administering to the subject an effective amount of a bacterial neuraminidase inhibitor, thereby treating the biofilm production-related disorder.

27. The method of claim 26, wherein the subject is a human, mouse, rat, bird, dog, cat, cow, horse, or pig.

28. The method of claim 26, wherein the disorder affects an epithelial surface, a mucosal surface, or a combination thereof.

29. The method of claim 28, wherein the surface is a lung surface.

30. The method of claim 26, wherein the biofilm production-related disorder is caused by a bacterium.

31. The method of claim 26, wherein the disorder is pneumonia, cystic fibrosis (CF), otitis media, or chronic obstructive pulmonary disease (COPD).

32. The method of claim 26, wherein the disorder is a medical device-related bacterial infection, the device being implanted or inserted into the subject.

33. The method of claim 30, wherein the bacterium comprises a Gram-negative or Gram-positive bacterium.

34. The method of claim 30, wherein the bacterium comprises Pseudomonas, Streptococcus, Haemophilus, Vibrio, Streptococcus pneumoniae, Haemophilus influenzae, Vibrio cholerae, or a combination thereof.

35. The method of claim 26, wherein the neuraminidase inhibitor is a compound comprising Formula (X), Formula (I), or Formula (A).

36. The method of claim 26, wherein the neuraminidase inhibitor is an antibody that specifically binds to the NanA protein of S. pneumoniae or a fragment thereof; an antisense RNA or antisense DNA that inhibits expression of a NanA polypeptide; a siRNA that specifically targets a NanA gene, a peptide comprising at least 10 amino acids of SEQ ID NO: 2 wherein the peptide competes with endogenous NanA for ligand binding; or a combination thereof.

37. A method for identifying a compound that modulates neuraminidase activity, the method comprising:
a) providing an electronic library of test compounds;
b) providing atomic coordinates for at least twenty amino acid residues for the active site of Streptococcus neuraminidase listed in Table 2, wherein the coordinates have a root mean square deviation therefrom, with respect to at least 50% of the Cα atoms, of not greater than about 2 Å, in a computer readable format;
c) converting the atomic coordinates into electrical signals readable by a computer processor to generate a three dimensional model of the neuraminidase;
d) performing a data processing method, wherein electronic test compounds from the library are superimposed upon the three dimensional model of the neuraminidase; and
e) determining which test compound fits into the binding pocket of the three dimensional model of the neuraminidase,
thereby identifying which compound would modulate the activity of the neuraminidase.

38. A method for identifying a compound that modulates neuraminidase activity, the method comprising:
a) providing an electronic library of test compounds;
b) providing atomic coordinates listed in Table 2 in a computer readable format for at least 5, 6, 7, 8, 9, 10, 11, or 12 amino acid residues located within about 10 Å of the neuraminidase active site, wherein the residues comprise 5 or more of the following residues: Arg347, Arg366, Asp372, Asp417, Ile442, Phe443, Phe565, Tyr590, Gln602, Glu647, Arg663, Tyr695, Tyr752, or Arg 721;
c) converting the atomic coordinates into electrical signals readable by a computer processor to generate a three dimensional model of the neuraminidase active site;
d) performing a data processing method, wherein electronic test compounds from the library are superimposed upon the three dimensional model of the neuraminidase active site; and
e) determining which test compound fits into the binding pocket of the three dimensional model of the neuraminidase active site,
thereby identifying which compound would modulate the activity of the neuraminidase.

39. The method of claim 37 or 38, further comprising:
f) obtaining or synthesizing the compound determined to bind to NanA or modulate the neuraminidase activity;
g) contacting a bacterium with the compound in vitro; and
h) determining whether the compound modulates neuraminidase activity using a biological assay.

40. The method of claim 39, wherein the bacterium is a Gram-negative or Gram-positive bacterium.

41. The method of claim 39, wherein the bacterium is Pseudomonas, Streptococcus, Haemophilus, Vibrio, Streptococcus pneumoniae, Haemophilus influenzae, or Vibrio cholerae.

42. The method of claim 39, wherein the biological assay comprises a biofilm assay, an adherence assay, or a combination thereof.

43. A compound identified by the method of claim 37 or claim 38, wherein the compound binds to the neuraminidase active site, and comes within 10 Å of amino acid residues listed in Table 3.

44. The compound of claim 43, wherein the compound inhibits or reduces biofilm formation.

45. The compound of claim 43, wherein the compound is a peptide that binds to a neuraminidase.

46. The compound of claim 45, wherein the peptide is an anti-neuraminidase antibody or a binding fragment thereof.

47. The compound of claim 45, wherein the peptide interacts with a protein having the amino acid sequence of SEQ ID NO: 2.

48. The compound of claim 45, wherein the compound interacts with a protein having the amino acid sequence of SEQ ID NO: 2.

49. A compound identified by the method of claim 39, wherein the compound binds to the neuraminidase active site, and comes within 10 Å of amino acid residues listed in Table 3.

50. The compound of claim 49, wherein the compound inhibits or reduces biofilm formation.

51. The compound of claim 49, wherein the compound is a peptide that binds to a neuraminidase.

52. The compound of claim 51, wherein the peptide is an anti-neuraminidase antibody or a binding fragment thereof.

53. The compound of claim 51, wherein the peptide interacts with a protein having the amino acid sequence of SEQ ID NO: 2.

54. The compound of claim 51, wherein the compound interacts with a protein having the amino acid sequence of SEQ ID NO: 2.
